(19) United States
(12) Patent Application Publication
(10) Pub. No.: US 2024/0408064 A1
(43) Pub. Date: Dec. 12, 2024

(54) RAS INHIBITORS, COMPOSITIONS AND METHODS OF USE THEREOF

(71) Applicant: GENETOLEAD INC., Laval, Québec (CA)

(72) Inventors: Luc FARMER, Montréal, Quebec (CA); Steven LAPLANTE, Laval, Quebec (CA); Yann AYOTTE, Blainville, Quebec (CA); Sacha Thierry LARDA, Laval, Quebec (CA); Saba ALAPOUR, Toronto, Ontario (CA); Simon WOO, Pierrefonds, Quebec (CA); David BENDAHAN, Côte-Saint-Luc, Quebec (CA); Maria DENK, Montréal, Québec (CA); Majid FARAHANI, Toronto, Ontario (CA); Mustapha IDDIR, Pointe-aux-Trembles, Québec (CA)

(73) Assignee: GENETOLEAD INC., Laval, Québec (CA)

(21) Appl. No.: 18/701,244

(22) PCT Filed: Oct. 14, 2022
(86) PCT No.: PCT/CA2022/051520

§ 371 Date: Apr. 14, 2024

Related U.S. Application Data
(60) Provisional application No. 63/262,585, filed on Oct. 15, 2021.

(51) Int. Cl. A61K 31/4045; A61K 31/4439; A61K 31/454; A61K 31/496; A61K 31/55; A61P 35/00; C07D 209/18; C07D 403/06; C07D 405/12; C07D 405/14

(52) U.S. Cl. CPC A61K 31/4045; A61K 31/4439; A61K 31/454; A61K 31/496; A61K 31/55; A61P 35/00; C07D 209/18; C07D 403/06; C07D 405/12; C07D 405/14

(57) ABSTRACT

There is provided a compound of formula (I), and its use for inhibiting RAS (wild type or mutant), for example HRAS, NRAS, and/or KRAS, and for the prevention or treatment of a disease or disorder associated with abnormal RAS activity, for example abnormal RAS activity caused by a mutation in RAS, including cancers with a mutated RAS.

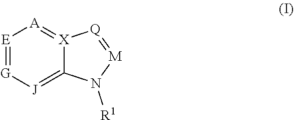

### CROSS REFERENCE TO RELATED APPLICATIONS

This application embodiments benefit, under 35 U.S.C. § 119(e), of U.S. provisional application Ser. No. 63/262,585, filed on Oct. 15, 2021. All documents above are incorporated herein in their entirety by reference.

### FIELD OF THE INVENTION

The present invention relates to novel compounds that are RAS inhibitors, for example HRAS inhibitors, NRAS inhibitors and/or KRAS inhibitors. More specifically, the present invention is concerned with novel bicyclic compounds and their use to treat e.g., cancers due related to the RAS protein, for example the HRAS protein, the NRAS protein, and/or the KRAS protein.

### BACKGROUND OF THE INVENTION

The RAS subfamily comprises the ubiquitously expressed human RAS proteins KRAS4A, KRAS4B (the two KRAS splice variants), HRAS, and NRAS, which are frequently mutated in cancer. These genes encode small GTPases that function as molecular regulators, controlling a broad spectrum of cellular activities, such as proliferation and cell survival. RAS proteins are considered molecular switches because they cycle between the “on” and “off” conformations, which are given by the binding of GTP and GDP, respectively. The transition between both states is regulated by two different protein families. The guanine nucleotide exchange factors (GEFs) promote GDP dissociation and GTP binding while the GTPase-activating proteins (GAPs) stimulate RAS intrinsic GTPase activity to switch off this signal.

High homology is shared by the three RAS proteins, except for the C-terminus hypervariable region, which is thought to confer the specific function of each protein. It has been reported that up to one-third of human cancers bears gain-of-function missense mutations that occur in the protein region that is identical among the four RAS proteins. Forty-four different point mutations have been described and 99.2% of them are located at codons 12, 13, and 61, but other non-canonical codons (such as 19, 117, or 146) are also mutated at low frequencies. All these canonical mutations prompt the loss of the intrinsic and/or the GAP-stimulated GTPase activity of RAS proteins, leading to a constitutively activated form of RAS. However, some non-canonical mutations, such as for example HRAS A146 mutations, do not impair RAS GTPase activity, but increase guanine nucleotide exchange.

Interestingly, the mutated isoform, as well as the codon position and the amino acid substitution varies among RAS proteins in human cancers, but the reason remains to be established. Regarding protein variability, KRAS is the most frequently mutated protein in human cancers, followed by NRAS and HRAS. Oncogenic alterations in KRAS are more frequent in patients with pancreatic carcinoma, colorectal tumors and lung malignancies. Mutations in HRAS can be found in dermatological malignancies and head and neck cancers, while NRAS mutations are common in melanomas and in some hematopoietic malignancies.

| RAS protein | Malignancies | Codon | Amino acid substitution |
|---|---|---|---|
| HRAS | Dermatological | Codon 12: GGC (Gly, G) | 12A, 12C, 12D, 12R, 12S, 12V |
| | Head and neck cancer | Codon 13: GGT (Gly, G) | 13C, 13D, 13R, 13S, 13V |
| | | Codon 61: CAG (Gln, Q) | 61H, 61K, 61L, 61P, 61R |
| KRAS | Pancreatic carcinoma | Codon 12: GGT (Gly, G) | 12A, 12C, 12D, 12R, 12S, 12V |
| | Colorectal cancer | Codon 13: GGC (Gly, G) | 13A, 13C, 13D, 13R, 13S, 13V |
| | Lung malignancies | Codon 61: CAA (Gln, Q) | 61E, 61R, 61K, 61L, 61P, 61R |
| NRAS | Melanomas | Codon 12: GGT(Gly, G) | 12A, 12C, 12D, 12R, 12S, 12V |
| | Hematopolatic | Codon 13: GGT (Gly, G) | 13A, 13C, 13D, 13R, 13S, 13V |
| | malignancies | Codon 61: CAA (Gln, Q) | 61E, 61H, 61K, 61L, 61P, 61R |

Amino acid substitutions identified at codon 12, 13, and 61 of each RAS protein, highlighting in red the most frequently observed. Gly and G [symbol] glycine; Gln and Q [symbol] glutamine; A, alanine; C, cysteine; D, aspartic acid; R, [symbol] S, [symbol] V, val [symbol] H, his [symbol] K, lysine; L, Leucine; P pro [symbol] E, glutamic acid. [symbol]
indicates data missing or illegible when filed

The mutations rates at each codon differ between the RAS proteins. While KRAS is commonly mutated at codon 12 with only few mutations occurring at codon 61, NRAS mutations are most frequently observed at codon 61. In addition, HRAS mutational rate is similar for both codons 12 and 61, displaying an intermediate mutational pattern between KRAS and NRAS.

Each of these codons can be substituted through a single-nucleotide change resulting in codons 12 and 13 changes from glycine to alanine, cysteine, aspartic acid, arginine, serine or valine and codon 61 from glutamine to glutamic acid, histidine, lysine, leucine, proline or arginine. In KRAS, the variations at codons 12 and 13, which are the most frequent mutations associated with this protein, result in G12D and G13D substitution, respectively. Similarly, the most common mutation in HRAS is the G12V substitution. As previously mentioned, NRAS has a mutation bias at codon 61, Q61R replacement at this position being the most frequent aberration.

The RASopathies are a clinically defined group of medical genetic syndromes caused by germline mutations in genes that encode components or regulators of the Ras/mitogen-activated protein kinase (MAPK) pathway. These disorders include neurofibromatosis type 1, Noonan syndrome, Noonan syndrome with multiple lentigines, capillary malformation-arteriovenous malformation syndrome, Costello syndrome, cardio-facio-cutaneous syndrome, and Legius syndrome. Because of the common underlying Ras/MAPK pathway dysregulation, the RASopathies exhibit numerous overlapping phenotypic features. The Ras/MAPK pathway plays an essential role in regulating the cell cycle and cellular growth, differentiation, and senescence, all of which are critical to normal development. Ras/MAPK pathway dysregulation has profound deleterious effects on both embryonic and later stages of development.

Of particular interest is the Costello syndrome which is a distinctive rare multisystem disorder comprising a characteristic coarse facial appearance, intellectual disabilities, and tumor predisposition. Although the diagnosis can be suspected clinically, confirmation requires identification of a heterozygous mutation in the proto-oncogene HRAS. In contrast to somatic oncogenic mutations in neoplasia, the Costello syndrome changes are typically introduced in the paternal germline. The predicted amino acid substitutions allow for constitutive or prolonged activation of the HRAS protein, resulting in dysregulation of the Ras/mitogen activated protein kinase pathway. Dysregulation of this signaling pathway is the disease mechanism shared among Costello syndrome and other RASopathies, including neurofibromatosis type 1, Noonan syndrome, cardio-facio-cutaneous syndrome, and Legius syndrome. The Ras/mitogen activated protein kinase pathway governs cell proliferation and differentiation, and its dysregulation affects cardiac and brain development, accounting for the significant overlap in physical and developmental differences and common medical problems among RASopathies. Unlike the genetically heterogeneous Noonan syndrome and cardio-facio-cutaneous syndrome, Costello syndrome is caused by HRAS mutations only. Patients, clinicians, and researchers may benefit from a multidisciplinary “RASopathy clinic,” which serves patients with more common conditions such as Noonan syndrome and neurofibromatosis and those affected by rare conditions such as Costello syndrome.

Despite more than three decades of intense effort, few anti-RAS therapies have reached or are about to reach clinical application. There is thus a need for the development of mutation-selective anti-RAS strategies.

### SUMMARY OF THE INVENTION

In accordance with the present invention, there is provided:
1. A compound of formula (I):

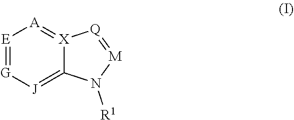

wherein:
   R¹ represents H, alkoxycarbonyl, alkylcarbonyl, Cₙalkyl, wherein n is 2 or more, or carbamoyl,
   A represents —C(R²)═,
   E represents —N═ or —C(R³)═,
   G represents —N═ or —C(R⁴)═,
   J represents —C(R⁵)═,
   X represents —C(R⁶)═,
   M represents —N═ or —C(R⁷)═,
   Q represents —N═ or —C(R⁸)═,
with the proviso that no more than four (4) of A, E, G, J, X, M, and Q represent —N═,
each of R⁴, R⁵, R⁶, and R⁸ independently represents H, a halogen atom, alkyl, alkenyl, alkynyl, alkenynyl, hydroxyl, —OR⁹, or -L-R¹⁰,
R² represents H, a halogen atom, alkyl, alkenyl, alkynyl, alkenynyl, alkyl-N(R⁹)₂, alkenyl-N(R⁹)₂, alkynyl-N(R⁹)₂, alkenynyl-N(R⁹)₂, hydroxyl, or —OR⁹,
R³ represents H, alkyl, alkenyl, alkynyl, alkenynyl, alkyl-N(R⁹)₂, alkenyl-N(R⁹)₂, alkynyl-N(R⁹)₂, alkenynyl-N(R⁹)₂, hydroxyl, or —OR⁹,
R⁷ represents H, a halogen atom, Cₙalkyl, wherein n is 2 or more, alkenyl, alkynyl, alkenynyl, alkyl-N(R⁹)₂, alkenyl-N(R⁹)₂, alkynyl-N(R⁹)₂, alkenynyl-N(R⁹)₂, hydroxyl, or —OR⁹,
R⁹ represents H, alkyl, alkenyl, alkynyl, or alkenynyl, each of the alkyl, alkenyl, alkynyl, and alkenynyl being optionally substituted with R¹⁶,
with the proviso that the compound of formula (I) comprises exactly one or exactly two -L-R¹⁰ group(s) identical or different from one another,
each L independently represents a covalent bond or a chain comprising any combination of the following:
   up to two —N(R¹¹)— group,
   up to one —C(═O)—, —O—C(═O)—, —C(═O)—O—, —SO₂—, or —SO— group, and
   up to five —C(R¹²)₂— groups, wherein the —C(R¹²)₂— groups can be adjacent to one another or separated
   by —N(R¹¹)—, —C(═O)—, and/or —S(═O)₂ groups,
wherein, in each -L-R¹⁰ group independently:
A) R¹⁰ represents:
   a 6-membered cycle selected from cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl, each of which comprising one or more nitrogen ring atoms as sole heteroatoms, and each of which being independently unsubstituted or substituted with:
      a halogen atom, —CN, hydroxyl, —N(R¹⁵)₂, —N(R¹⁵)—C(═O)—R¹⁸, —C(═O)—N(R¹⁵)₂, —C(═O)—R¹⁷, —C(═O)—OR¹⁷, —OR¹⁵, —O—C(═O)—R¹⁷, —SO₂—R¹⁵, —SO—R¹⁵, —N(R¹⁵)₂—SO₂—R¹⁵, or —N(R¹⁵)₂—SO—R¹⁵, or alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, or heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R¹⁶,
   or
   aryl or heteroaryl, each of which independently unsubstituted or substituted with, in a position other than in the position immediately next to the ring atom attached to L:
      alkyl-N(R¹⁵)₂, alkenyl-N(R¹⁵)₂, alkynyl-N(R¹⁵)₂, alkenynyl-N(R¹⁵)₂, —N(R¹⁵)₂, —N(R¹⁵)—C(═O)—R¹⁵, —C(═O)—N(R¹⁵)₂, —C(═O)—R¹⁵, —C(═O)—OR¹⁷, —OR¹⁸, —O—C(═O)—R¹⁵, —SO₂—R¹⁵, —SO—R¹⁵, —N(R¹⁵)₂—SO₂—R¹⁵, —N(R¹⁵)₂—SO—R¹⁵, or
      cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of independently unsubstituted or substituted with one or more R¹⁶,
   with the proviso that the aryl or heteroaryl are substituted with no more than one —OR¹⁸ group, and
   with the proviso that -L-R¹⁰ is not

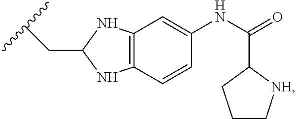 [SMILES: *CC1Nc2ccc(NC(=O)C3CCCN3)cc2N1]

L represents a chain of at most 5 atoms in length,
each R¹¹ independently represents H, or
   alkyl, alkenyl, alkynyl, or alkenynyl, each of which independently unsubstituted of substituted with one or more R³⁰,
each R¹² independently represents H, —C≡N, —N(R¹⁵)₂, -T-COOR¹⁴, or
   alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰,
B) R¹⁰ represents —COOH,
   L represents a chain that is at most 3 atoms in length, and that is not —CH₂—CH(NH₂)—,
   any R¹¹ independently represents H, or
      alkyl, alkenyl, alkynyl, or alkenynyl, each of which independently unsubstituted or substituted with one or more R³⁰, and
   any R¹² independently represents H, —C≡N, —N(R¹⁵)₂, —C(═O)—R¹⁵, or
      alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰,
C) R¹⁰ is attached to a nitrogen atom of a —N(R¹¹)— group that ends the chain in L, and R¹⁰ and the R¹¹ of the —N(R¹¹)— group that ends the chain in L together with the nitrogen atom to which they are attached form a heteroaryl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl, each of which independently unsubstituted or substituted with:
   a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR²⁰, —OR¹⁵, —O—C(═O)—R¹⁵, —SO₂—R¹⁵, —SO—R¹⁵, —N(R¹⁵)₂—SO₂—R¹⁵, —N(R¹⁵)₂—SO—R¹⁵, or alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R²¹, with the proviso that when R¹⁰ and R¹¹ together with the nitrogen atom to which they are attached form morpholinyl, the morpholinyl is free of a substituent containing a —C(═O)— group, with the proviso that when R¹⁰ and R¹¹ together with the nitrogen atom to which they are attached form piperazinyl substituted with a —C(═O)—O—CH₃ group, the —C(═O)—O—CH₃ group is in position 2, with the proviso that when R¹⁰ and R¹¹ together with the nitrogen atom to which they are attached form piperazinyl N-substituted with —O-heterocycloalkyl, the heterocycloalkyl is other than oxalanyl,
   the chain in L is at most 4 atoms in length and ends with the —N(R¹¹)— group to which R¹⁰ is attached,
   with the proviso that -L-R¹⁰ is not

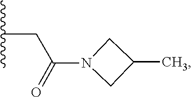 [SMILES: *CC(=O)N1CC(C)C1]

any other R¹¹ independently represents H, or
   alkyl, alkenyl, alkynyl, or alkenynyl, each of which independently unsubstituted or substituted with one or more R³¹ and
any R¹² independently represents H, —C≡N, —N(R¹⁵)₂, or
   alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, eterocycloalkenynonyl, aryl, or heteroaryl, each of which independently unsubstituted or substituted with one or more R³¹,
   with the proviso that when one R¹² on a given carbon atom is methyl, any other R¹² on said given carbon atom is other than methyl,
or
   D) R¹⁰ represents H, a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR¹⁵, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵, or alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each independently unsubstituted or substituted with one or more R¹⁶,
      the chain in L is at most 3 atoms in length, contains at least one —C(R¹²)₂— group, and ends with a —N(R¹¹)— group,
      the R¹¹ of the —N(R¹¹)— group that ends the chain in L and one R¹² of said at least one —C(R¹²)₂— group together with the one or more atoms to which they are attached and any atom(s) between said one or more atoms form heterocycloalkyl, heterocycloalkanonyl, heterocycloalkenonyl, heterocycloalkynonyl, heterocycloalkenynonyl, or heteroaryl, each of which being independently unsubstituted or substituted with:
         a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR¹⁵, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵, alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which being independently unsubstituted or substituted with one or more R¹⁶,
         with the proviso that when said one R¹¹ and said one R¹² form pyrrolidinyl, the pyrrolidinyl is substituted,
         with the proviso that when the heterocycloalkyl is piperidinyl, R¹⁰ is other than 2,4-difluorophenylcarbonyl substituent,
      any other R¹¹ independently represent H or
         alkyl, alkenyl, alkynyl, alkenynyl, each of which independently unsubstituted or substituted with one or more R³¹, and
      any other R¹² independently represent H, —C≡N, or —N(R¹⁵)₂,
         alkyl, alkenyl, alkynyl, or alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³¹,
      with the proviso that -L-R¹⁰ is not

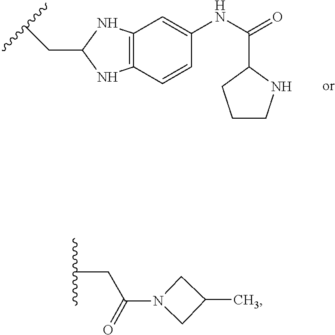

wherein R¹⁴ represents H, alkyl, alkenyl, alkynyl, or alkenynyl,
wherein T represents a covalent bond, alkylene, alkenylene, alkynylene, or alkenynylene,
wherein each R³⁰ independently represents:
a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR¹⁵, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵,
alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R¹⁶,
wherein each R³¹ independently represents:
a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR²⁰, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵,
alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R¹⁶,
wherein each R¹⁵ independently represents H, or
alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R¹⁶
wherein each R¹⁷ independently represents cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, aryl, or heteroaryl, each of which independently unsubstituted or substituted with one or more R¹⁶,
wherein each R²⁰ independently represents alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, aryl, or heteroaryl, or, each of which independently unsubstituted or substituted with one or more R¹⁶
wherein each R¹⁸ independently represents H, or
alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, aryl, or heteroaryl, each of which independently unsubstituted or substituted with one or more R¹⁹,
wherein each R¹⁶ independently represents a halogen atom, alkoxy, alkylcarbonyl, alkenylcarbonyl, alkynylcarbonyl, alkenynylcarbonyl, alkoxycarbonyl, alkyl, alkenyl, alkynyl, alkenynyl, carbamoyl, alkylcarbamoyl, alkenylcarbamoyl, alkynylcarbamoyl, alkenynylcarbamoyl, aryl (optionally substituted with one or more halogen atoms), heteroaryl (optionally substituted with one or more halogen atoms), —CN, carboxyl, hydroxyl, amido, alkylamido, dialkylamido, alkenylamido, dialkenylamido, alkynylamido, dialkynylamido, alkenynylamido, dialkenynylamido, amino, alkylamino, dialkylamino, alkenylamino, dialkenylamino, alkynylamino, dialkynylamino, alkenynylamino, dialkenynylamino, alkylsulfonylamino, alkenylsulfonylamino, alkynylsulfonylamino, or alkenynylsulfonylamino,
wherein each R¹⁹ independently represents a halogen atom, alkylcarbonyl, alkenylcarbonyl, alkynylcarbonyl, alkenynylcarbonyl, alkoxycarbonyl, alkyl, alkenyl, alkynyl, alkenynyl, carbamoyl, alkylcarbamoyl, alkenylcarbamoyl, alkynylcarbamoyl, alkenynylcarbamoyl, aryl (optionally substituted with one or more halogen atoms), heteroaryl (optionally substituted with one or more halogen atoms), —CN, carboxyl, hydroxyl, amido, alkylamido, dialkylamido, alkenylamido, dialkenylamido, alkynylamido, dialkynylamido, alkenynylamido, dialkenynylamido, amino, alkylamino, dialkylamino, alkenylamino, dialkenylamino, alkynylamino, dialkynylamino, alkenynylamino, dialkenynylamino, alkylsulfonylamino, alkenylsulfonylamino, alkynylsulfonylamino, or alkenynylsulfonylamino
wherein each R²¹ independently represents a halogen atom, alkoxy, alkylcarbonyl, alkenylcarbonyl, alkynylcarbonyl, alkenynylcarbonyl, alkoxycarbonyl, alkyl, alkenyl, alkynyl, alkenynyl, carbamoyl, alkylcarbamoyl, alkenylcarbamoyl, alkynylcarbamoyl, alkenynylcarbamoyl, aryl (optionally substituted with one or more halogen atoms), heteroaryl (optionally substituted with one or more halogen atoms), —CN, hydroxyl, amido, alkylamido, dialkylamido, alkenylamido, dialkenylamido, alkynylamido, dialkynylamido, alkenynylamido, dialkenynylamido, amino, alkylamino, dialkylamino, alkenylamino, dialkenylamino, alkynylamino, dialkynylamino, alkenynylamino, dialkenynylamino, alkylsulfonylamino, alkenylsulfonylamino, alkynylsulfonylamino, or alkenynylsulfonylamino,
or a pharmaceutically acceptable salt, ester, solvate, isomer, or tautomer thereof.
2. The compound of embodiment 1, comprising exactly one -L-R¹⁰ group.
3. The compound of embodiment 1, comprising exactly two -L-R¹⁰ group.
4. The compound of any one of embodiments 1 to 3, wherein R¹ represents H.
5. The compound of any one of embodiments 1 to 4, wherein A represents —CH═.
6. The compound of any one of embodiments 1 to 5, wherein E represents —C(R³)═, preferably —C(H)═.
7. The compound of any one of embodiments 1 to 6, wherein G represents —C(R⁴)═, preferably wherein R⁴ represents a halogen atom, more preferably F.
8. The compound of any one of embodiments 1 to 7, wherein J represents —CH═.
9. The compound of any one of embodiments 1 to 8, wherein X represents —C(H)═.
10. The compound of any one of embodiments 1 to 9, wherein M represents —C(R⁷)═, wherein R^{y7} preferably represents H.
11. The compound of any one of embodiments 1 to 9, wherein M represents —N═.
12. The compound of any one of embodiments 1 to 11, wherein Q represents —C(R⁸)═, preferably —C(-L-R¹⁰)═.
13. The compound of any one of embodiments 1 to 12, wherein A)
   R¹⁰ represents:
      a 6-membered cycle selected from cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl, each of which comprising one or more nitrogen ring atoms as sole heteroatoms, and each of which being independently unsubstituted or substituted with
         a halogen atom, —CN, hydroxyl, —N(R¹⁵)₂, —N(R¹⁵)—C(═O)—R¹⁸, —C(═O)—N(R¹⁵)₂, —C(═O)—R¹⁷, —C(═O)—OR¹⁷, —OR¹⁵, —O—C(═O)—R¹⁷, —SO₂—R¹⁵, —SO—R¹⁵, —N(R¹⁵)₂—SO₂—R¹⁵, or —N(R¹⁵)₂—SO—R¹⁵, or
         alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, or heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R¹⁶,
      or
      aryl or heteroaryl, each of which independently unsubstituted or substituted with, in a position other than in the position immediately next to the ring atom attached to L:
         alkyl-N(R¹⁵)₂, alkenyl-N(R¹⁵)₂, alkynyl-N(R¹⁵)₂, alkenynyl-N(R¹⁵)₂, —N(R¹⁵)₂, —N(R¹⁵)—C(═O)—R¹⁵, —C(═O)—N(R¹⁵)₂, —C(═O)—R¹⁵, —C(═O)—OR¹⁷, —OR¹⁸, —O—C(═O)—R¹⁵, —SO₂—R¹⁵, —SO—R¹⁵, —N(R¹⁵)₂—SO₂—R¹⁵, —N(R¹⁵)₂—SO—R¹⁵, or
         cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of independently unsubstituted or substituted with one or more R¹⁶,
      with the proviso that the aryl or heteroaryl are substituted with no more than one —OR¹⁸ group, and
      with the proviso that -L-R¹⁰ is not

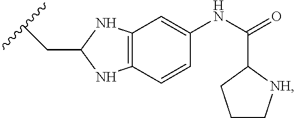 [SMILES: *CC1Nc2ccc(NC(=O)C3CCCN3)cc2N1]

L represents a chain of at most 5 atoms in length,
   each R¹¹ independently represents H, or
   alkyl, alkenyl, alkynyl, or alkenynyl, each of which independently unsubstituted of substituted with one or more R³⁰,
each R¹² independently represents H, —C≡N, —N(R¹⁵)₂, -T-COOR¹⁴, or
   alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰.
14. The compound of embodiment 13, wherein M represents —C(R⁷)═,
15. The compound of embodiment 13 or 14, wherein R⁷ represents H or alkyl-N(R⁹)₂, preferably wherein both R⁹ in alkyl-N(R⁹)₂ represent alkyl, preferably methyl.
16. The compound of embodiment 13 or 14, wherein R⁷ represents H or alkyl (which is preferably methyl), and preferably R⁷ represents H.
17. The compound of any one of embodiments 13 to 16, wherein the chain in L is at most 4 atoms in length, preferably at most 3 atoms in length.
18. The compound of any one of embodiments 13 to 17, wherein the chain in L is at least 1 atom in length, preferably at least 2 atoms in length.
19. The compound of any one of embodiments 13 to 18, wherein the chain in L is 3 atoms in length
20. The compound of any one of embodiments 13 to 19, wherein L represents:
   —C(R¹²)₂—N(R¹¹)—,
   —C(R¹²)₂—C(═O)—N(R₁₁)—,
   —C(R¹²)₂—N(R¹¹)—C(═O)—,
   —C(R¹²)₂—C(R¹²)₂—N(R¹¹)—C(═O)—,
   —C(R¹²)₂—C(R¹²)₂—C(═O)—N(R¹¹)—,
   —C(R¹²)₂—N(R¹¹)—C(═O)—C(R¹²)₂—,
   —C(R¹²)₂—C(═O)—N(R¹¹)—C(R¹²)₂—,
   —C(R¹²)₂—C(═O)—N(R¹¹)—C(R¹²)₂—C(R¹²)₂—, or
   —C(R¹²)₂—N(R¹¹)—C(═O)—C(R¹²)₂—C(R¹²)₂—,
   preferably:
   —C(R¹²)₂—N(R¹¹)—,
   —C(R¹²)₂—C(═O)—N(R¹¹)—,
   —C(R¹²)₂—C(R¹²)₂—N(R¹¹)—C(═O)—,
   —C(R¹²)₂—C(═O)—N(R¹¹)—C(R¹²)₂— or
   —C(R¹²)₂—C(═O)—N(R¹¹)—C(R¹²)₂—C(R¹²)₂—,
   and most preferably —C(R¹²)₂—C(═O)—N(R¹¹)—.
21. The compound of any one of embodiments 13 to 20, wherein L is free of a —N(R¹¹)— group in which R¹¹ forms a cycle with R¹⁰ or R¹².
22. The compound of any one of embodiments 13 to 21, being free of a R¹² that represents -T-COOH, preferably -T-COOR¹⁴.
23. The compound of any one of embodiments 13 to 22, wherein all R¹² represent H.
24. The compound of any one of embodiments 13 to 22, wherein one R¹² on a carbon atom is H and the other R¹² on the same carbon atom is —C≡N, alkyl or -T-COOR¹⁴, with the proviso that when said other R¹² is -T-COOR¹⁴, R¹⁰ represents an aryl or a heteroaryl.
25. The compound of any one of embodiments 13 to 22, wherein one R¹² in L is —C≡N or —COOH, and all the others R¹² in L are H, with the proviso that when said one R¹² is —COOH, R¹⁰ represents an aryl or a heteroaryl.
26. The compound of any one of embodiments 13 to 25, wherein, when R¹⁰ represents an aryl or a heteroaryl, preferably when L is a chain of 5 atoms in length, one R¹² on a first carbon atom in L is —C≡N, one R¹² on a second carbon atom in L is -T-COOR¹⁴, and all others R¹² in L are H.
27. The compound of any one of embodiments 13 to 26, wherein R¹¹ independently represents H or alkyl, alkenyl, alkynyl, alkenynyl, preferably H or alkyl (which is preferably methyl), and more preferably H.
28. The compound of any one of embodiments 13 to 27, wherein T is a covalent bond.
29. The compound of any one of embodiments 13 to 28, wherein R¹⁴ is H.
30. The compound of any one of embodiments 13 to 29, wherein —C(R¹²)₂—C(═O)—N(R¹¹)— represents —CH₂—C(═O)—N(R¹¹)—, or —CH(CN)—C(═O)—N(R¹¹)—.
31. The compound of any one of embodiments 13 to 30, wherein:
   —C(R¹²)₂—N(R¹¹)— represents —CH₂—NH—,
   —C(R¹²)₂—C(═O)—N(R¹¹)— represents —CH₂—C(═O)—NH— or —CH(CN)—C(═O)—NH,
   —C(R¹²)₂—C(R¹²)₂—N(R¹¹)—C(═O)— represents —CH₂—CH₂—N(H)—C(═O)—,
   —C(R¹²)₂—C(═O)—N(R¹¹)—C(R¹²)₂— represents —CH₂—C(═O)—N(alkyl)-CH(alkyl)- (in which both alkyls are
   preferably C₁₋₆ alkyl, more preferably methyl), and/or
   —C(R¹²)₂—C(═O)—N(R¹¹)—C(R¹²)₂—C(R¹²)₂—
   represents —CH₂—C(═O)—NH—CH₂—CH₂— or —CH(C≡N)—C(═O)—NH—CH(—COOH)—CH₂—,
   with the proviso that when —C(R¹²)₂—C(═O)—N(R¹¹)—C(R¹²)₂—C(R¹²)₂—
   represents —CH₂—C(═O)—NH—CH₂—CH₂— or —CH(C≡N)—C(═O)—NH—CH(—COOH)—CH₂—, R¹⁰ represents an aryl or a heteroaryl.
32. The compound of any one of embodiments 13 to 31, wherein R¹⁰ represents:
   a 6-membered cycle selected from heterocycloalkyl, heterocycloalkenyl, heterocycloalkynyl, or heterocycloalkenynyl, each of which comprising one or more nitrogen ring atoms as sole heteroatoms, and each of which being independently unsubstituted or substituted as described in embodiment 1,
   or
   aryl or heteroaryl, each of which independently unsubstituted or substituted as described in embodiment 1, preferably
      a 6-membered cycle that is a heterocycloalkyl comprising one or more nitrogen ring atoms as sole heteroatoms, being unsubstituted or substituted as described in embodiment 1,
      or
      aryl or heteroaryl, each of which independently unsubstituted or substituted as described in embodiment 1.
33. The compound of any one of embodiments 13 to 32, wherein R¹⁰ represents a heterocycloalkyl, preferably piperidinyl (preferably piperidin-4-yl) or piperazinyl (preferably piperazin-1-yl), each of which independently unsubstituted or substituted as described in embodiment 1.
34. The compound of any one of embodiments 13 to 33, wherein, when R¹⁰ represents a 6-membered cycle, the 6-membered cycle is unsubstituted or substituted with alkyl, preferably C₁₋₆ alkyl, more preferably C₁₋₃ alkyl, preferably methyl, ethyl or isopropryl.
35. The compound of any one of embodiments 13 to 32, wherein R¹⁰ represents an aryl, preferably phenyl, unsubstituted or substituted as described in embodiment 1.
36. The compound of any one of embodiments 13 to 32 and 35, wherein the aryl is unsubstituted or substituted with one or more, preferably one, of:
   —N(R¹⁵)—C(═O)—R¹⁵, preferably —NH—C(═O)—R¹⁵, preferably wherein R¹⁵ is alkyl (preferably methyl) unsubstituted or substituted with one or more (preferably one)
      amino, alkylamino, dialkylamino, preferably dialkylamino, preferably wherein the alkyl is C₁₋₆ alkyl, preferably C₁₋₃ alkyl, preferably methyl,
   —OR¹⁸, preferably wherein R¹⁸ is H or alkyl unsubstituted or substituted with one or more (preferably one) amino, alkylamino, dialkylamino, preferably dialkylamino, preferably wherein the alkyl is C₁₋₆ alkyl, preferably C₁₋₃ alkyl, preferably methyl, or
   alkyl-N(R¹⁵)₂, preferably wherein the alkyl is C₁₋₆ alkyl, preferably C₁₋₃ alkyl, preferably methyl; preferably wherein R¹⁵ is alkyl, preferably C₁₋₆ alkyl, preferably C₁₋₃ alkyl, preferably methyl.
37. The compound of any one of embodiments 13 to 32, wherein R¹⁰ represents a heteroaryl, preferably pyridinyl (preferably pyridin-3-yl or pyridin-4-yl) or pyrimidinyl (preferably pyrimidin-2-yl), each of which independently unsubstituted or substituted as described in embodiment 1.
38. The compound of any one of embodiments 13 to 32 and 37, wherein the heteroaryl is unsubstituted.
39. The compound of embodiment 1, being:

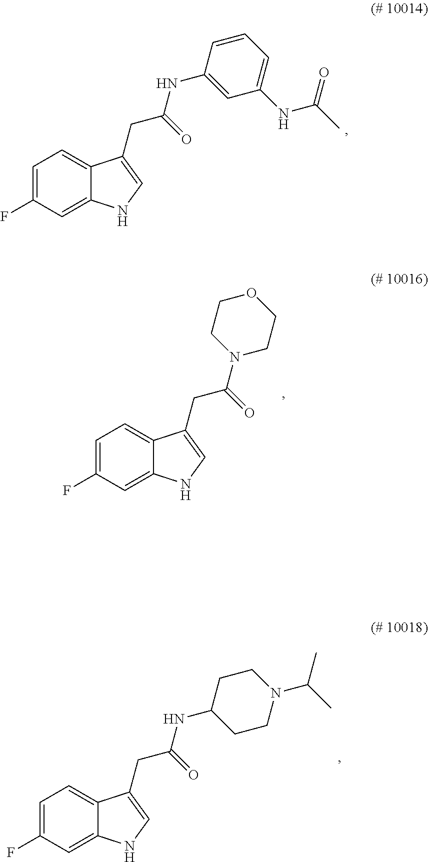 [SMILES: CC(=O)Nc1cccc(NC(=O)Cc2c[nH]c3cc(F)ccc23)c1 ; O=C(Cc1c[nH]c2cc(F)ccc12)N1CCOCC1 ; CC(C)N1CCC(NC(=O)Cc2c[nH]c3cc(F)ccc23)CC1]

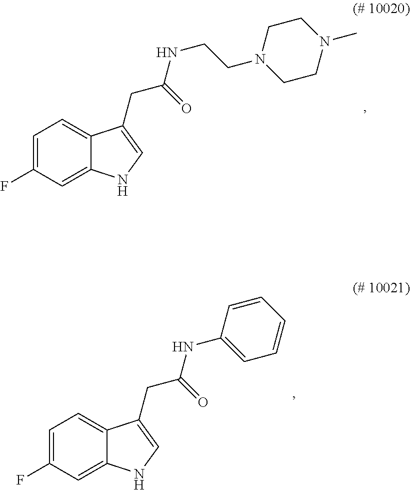 [SMILES: CN1CCN(CCNC(=O)Cc2c[nH]c3cc(F)ccc23)CC1 ; O=C(Cc1c[nH]c2cc(F)ccc12)Nc1ccccc1]

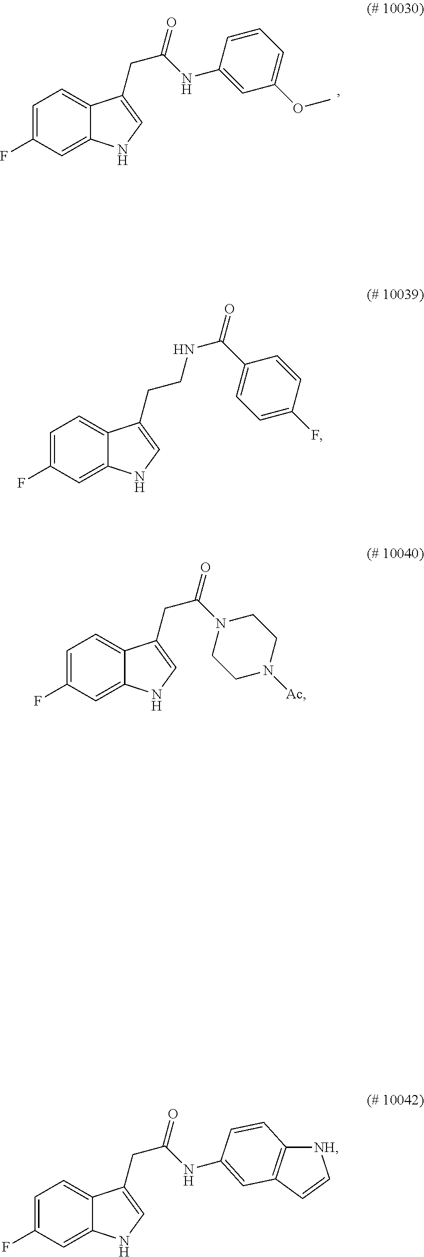 [SMILES: COc1cccc(NC(=O)Cc2c[nH]c3cc(F)ccc23)c1 ; O=C(NCCc1c[nH]c2cc(F)ccc12)c1ccc(F)cc1 ; CC(=O)N1CCN(C(=O)Cc2c[nH]c3cc(F)ccc23)CC1 ; O=C(Cc1c[nH]c2cc(F)ccc12)Nc1ccc2[nH]ccc2c1]

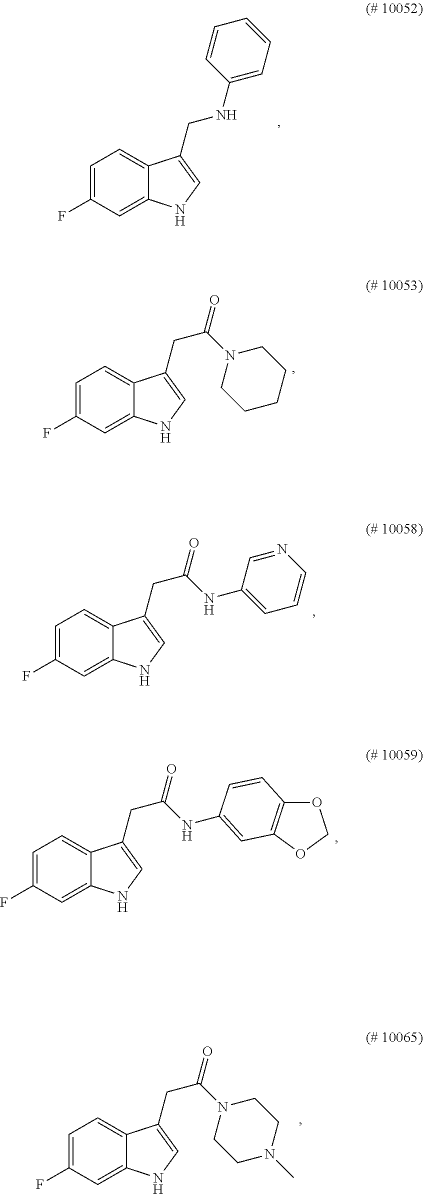 [SMILES: Fc1ccc2c(CNc3ccccc3)c[nH]c2c1 ; O=C(Cc1c[nH]c2cc(F)ccc12)N1CCCCC1 ; O=C(Cc1c[nH]c2cc(F)ccc12)Nc1cccnc1 ; O=C(Cc1c[nH]c2cc(F)ccc12)Nc1ccc2c(c1)OCO2 ; CN1CCN(C(=O)Cc2c[nH]c3cc(F)ccc23)CC1]

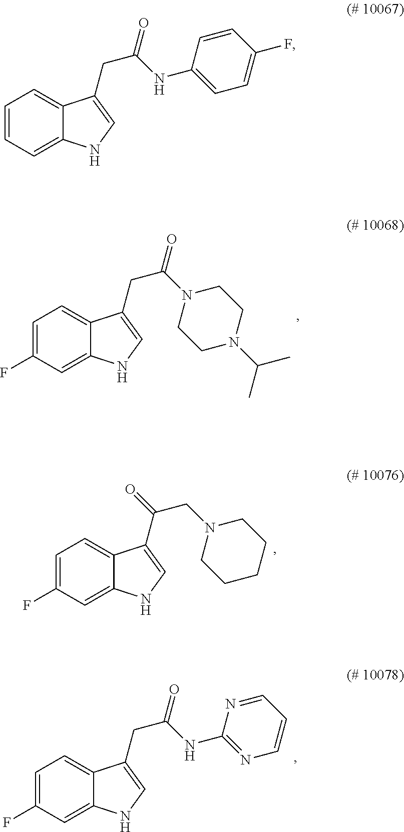 [SMILES: O=C(Cc1c[nH]c2ccccc12)Nc1ccc(F)cc1 ; CC(C)N1CCN(C(=O)Cc2c[nH]c3cc(F)ccc23)CC1 ; O=C(CN1CCCCC1)c1c[nH]c2cc(F)ccc12 ; O=C(Cc1c[nH]c2cc(F)ccc12)Nc1ncccn1]

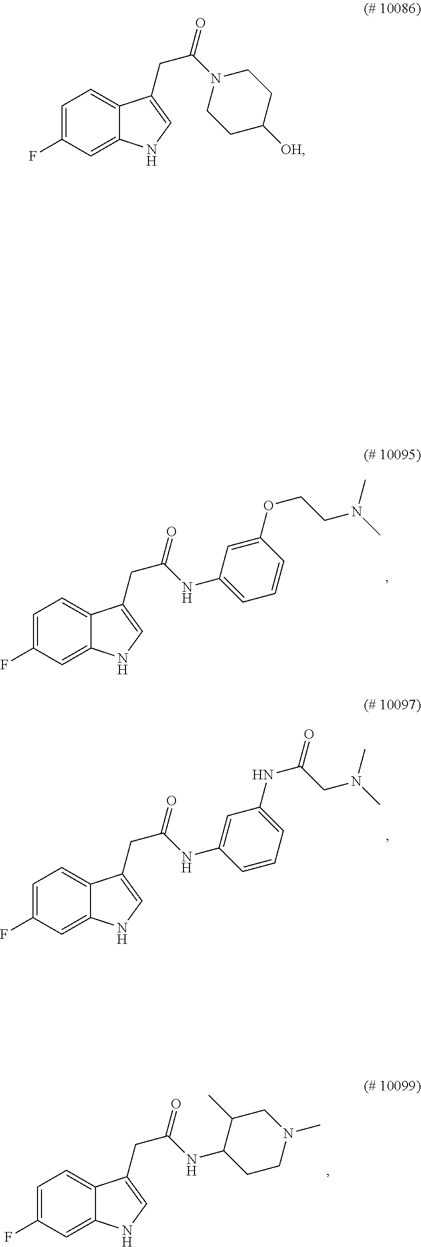 [SMILES: O=C(Cc1c[nH]c2cc(F)ccc12)N1CCC(O)CC1 ; CN(C)CCOc1cccc(NC(=O)Cc2c[nH]c3cc(F)ccc23)c1 ; CN(C)CC(=O)Nc1cccc(NC(=O)Cc2c[nH]c3cc(F)ccc23)c1 ; CC1CN(C)CCC1NC(=O)Cc1c[nH]c2cc(F)ccc12]

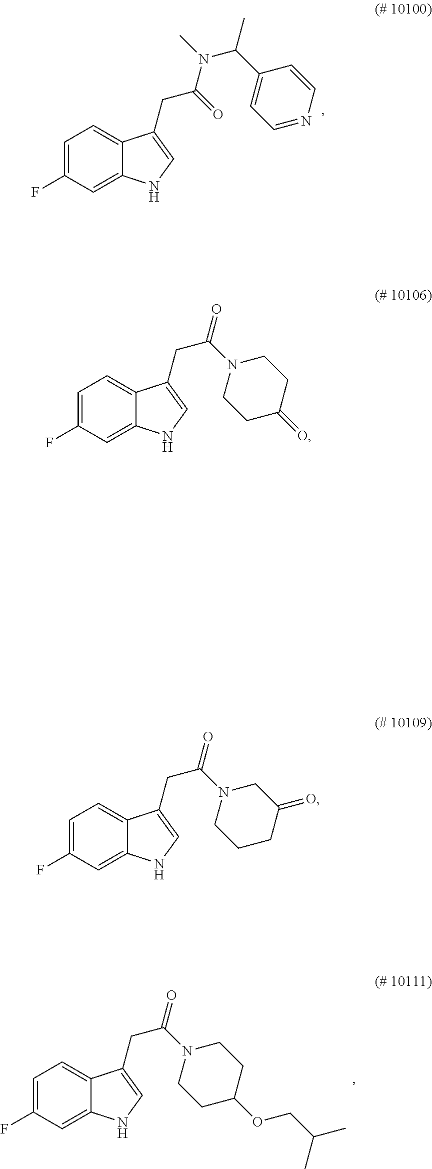 [SMILES: CC(c1ccncc1)N(C)C(=O)Cc1c[nH]c2cc(F)ccc12 ; O=C1CCN(C(=O)Cc2c[nH]c3cc(F)ccc23)CC1 ; O=C1CCCN(C(=O)Cc2c[nH]c3cc(F)ccc23)C1 ; CC(C)COC1CCN(C(=O)Cc2c[nH]c3cc(F)ccc23)CC1]

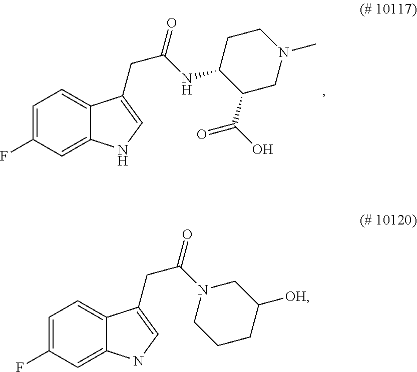 [SMILES: CN1CC[C@@H](NC(=O)Cc2c[nH]c3cc(F)ccc23)[C@@H](C(=O)O)C1 ; O=C(Cc1c[nH]c2cc(F)ccc12)N1CCCC(O)C1]

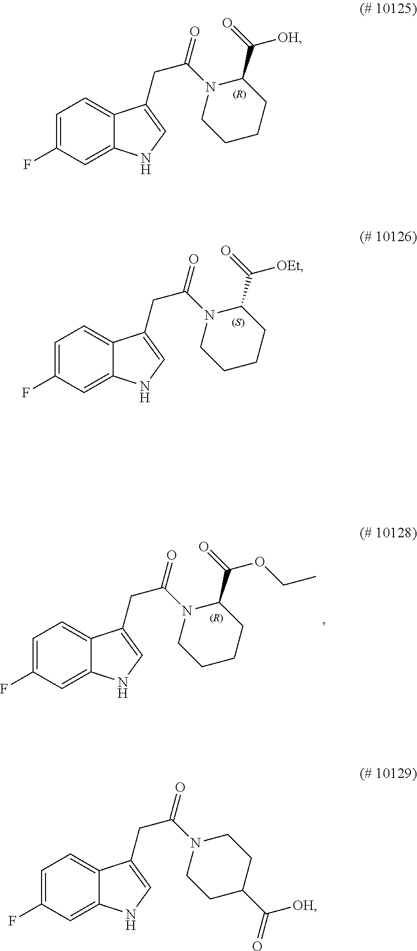

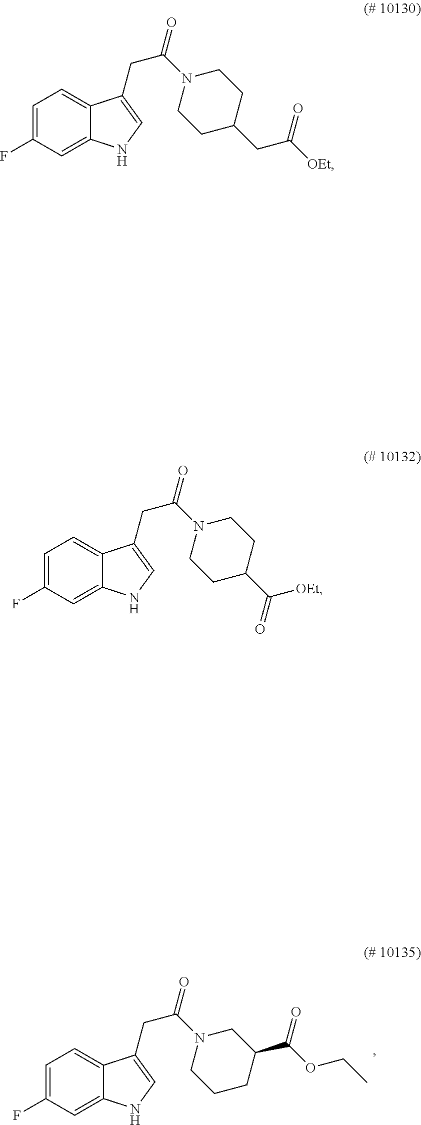 [SMILES: CCOC(=O)CC1CCN(C(=O)Cc2c[nH]c3cc(F)ccc23)CC1 ; CCOC(=O)C1CCN(C(=O)Cc2c[nH]c3cc(F)ccc23)CC1 ; CCOC(=O)[C@H]1CCCN(C(=O)Cc2c[nH]c3cc(F)ccc23)C1]

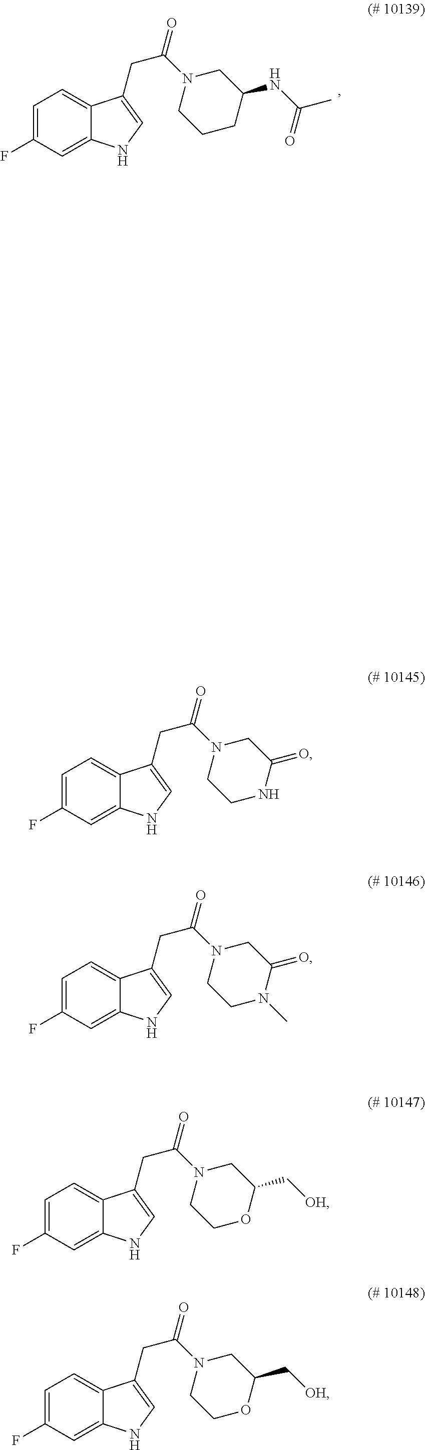 [SMILES: CC(=O)N[C@H]1CCCN(C(=O)Cc2c[nH]c3cc(F)ccc23)C1 ; O=C1CN(C(=O)Cc2c[nH]c3cc(F)ccc23)CCN1 ; CN1CCN(C(=O)Cc2c[nH]c3cc(F)ccc23)CC1=O ; O=C(Cc1c[nH]c2cc(F)ccc12)N1CCO[C@@H](CO)C1 ; O=C(Cc1c[nH]c2cc(F)ccc12)N1CCO[C@H](CO)C1]

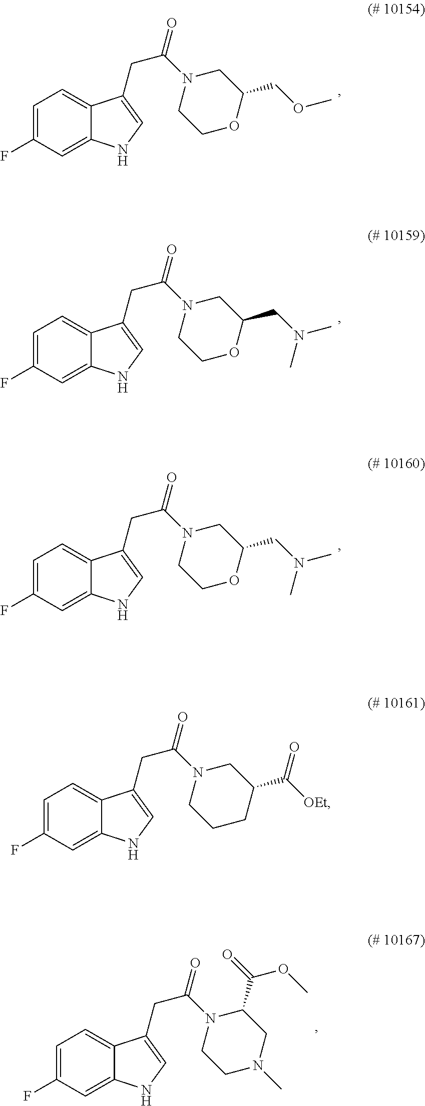 [SMILES: COC[C@H]1CN(C(=O)Cc2c[nH]c3cc(F)ccc23)CCO1 ; CN(C)C[C@@H]1CN(C(=O)Cc2c[nH]c3cc(F)ccc23)CCO1 ; CN(C)C[C@H]1CN(C(=O)Cc2c[nH]c3cc(F)ccc23)CCO1 ; CCOC(=O)[C@@H]1CCCN(C(=O)Cc2c[nH]c3cc(F)ccc23)C1 ; COC(=O)[C@@H]1CN(C)CCN1C(=O)Cc1c[nH]c2cc(F)ccc12]

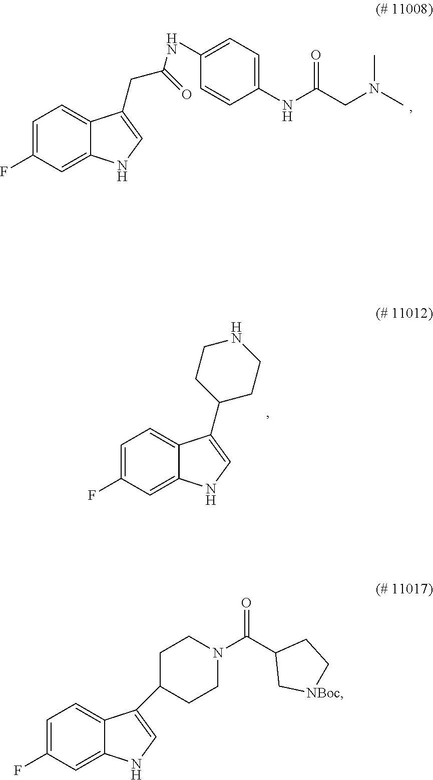 [SMILES: CN(C)CC(=O)Nc1ccc(NC(=O)Cc2c[nH]c3cc(F)ccc23)cc1 ; Fc1ccc2c(C3CCNCC3)c[nH]c2c1 ; CC(C)(C)OC(=O)N1CCC(C(=O)N2CCC(c3c[nH]c4cc(F)ccc34)CC2)C1]

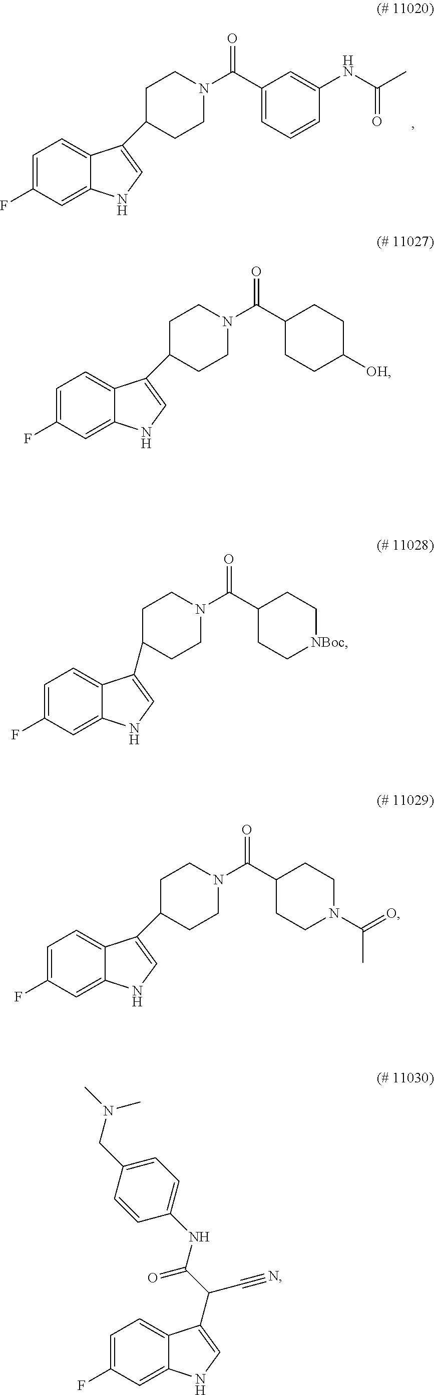 [SMILES: CC(=O)Nc1cccc(C(=O)N2CCC(c3c[nH]c4cc(F)ccc34)CC2)c1 ; O=C(C1CCC(O)CC1)N1CCC(c2c[nH]c3cc(F)ccc23)CC1 ; CC(C)(C)OC(=O)N1CCC(C(=O)N2CCC(c3c[nH]c4cc(F)ccc34)CC2)CC1 ; CC(=O)N1CCC(C(=O)N2CCC(c3c[nH]c4cc(F)ccc34)CC2)CC1 ; CN(C)Cc1ccc(NC(=O)C(C#N)c2c[nH]c3cc(F)ccc23)cc1]

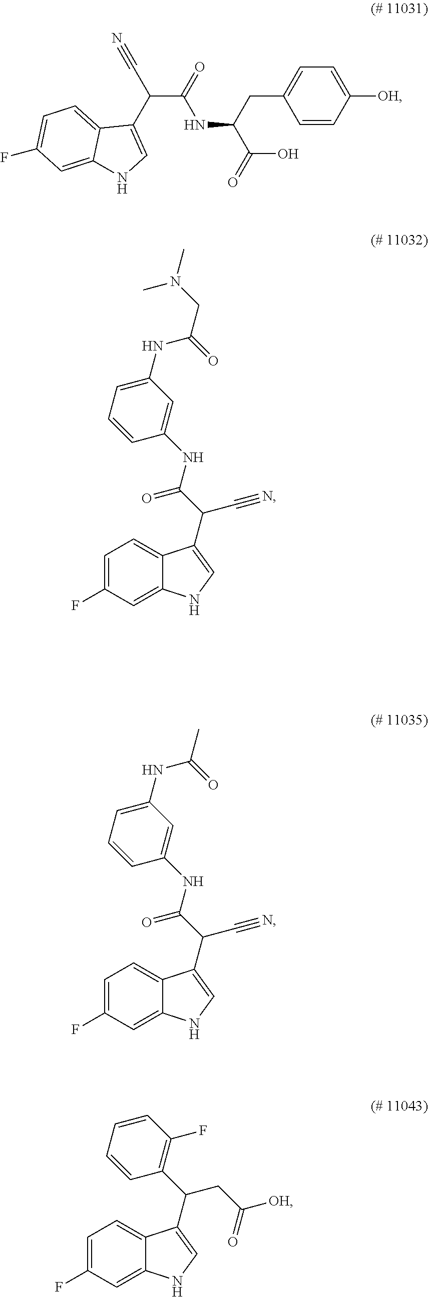 [SMILES: N#CC(C(=O)N[C@@H](Cc1ccc(O)cc1)C(=O)O)c1c[nH]c2cc(F)ccc12 ; CN(C)CC(=O)Nc1cccc(NC(=O)C(C#N)c2c[nH]c3cc(F)ccc23)c1 ; CC(=O)Nc1cccc(NC(=O)C(C#N)c2c[nH]c3cc(F)ccc23)c1 ; O=C(O)CC(c1ccccc1F)c1c[nH]c2cc(F)ccc12]

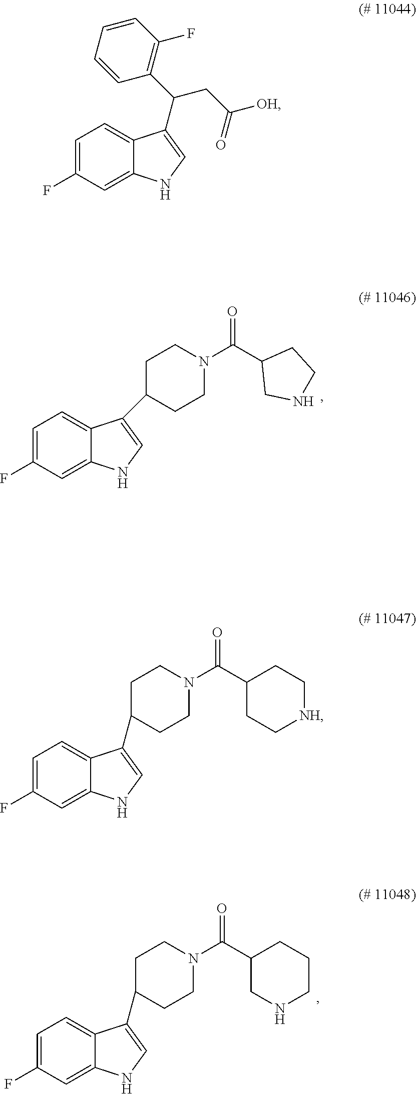 [SMILES: O=C(O)CC(c1ccccc1F)c1c[nH]c2cc(F)ccc12 ; O=C(C1CCNC1)N1CCC(c2c[nH]c3cc(F)ccc23)CC1 ; O=C(C1CCNCC1)N1CCC(c2c[nH]c3cc(F)ccc23)CC1 ; O=C(C1CCCNC1)N1CCC(c2c[nH]c3cc(F)ccc23)CC1]

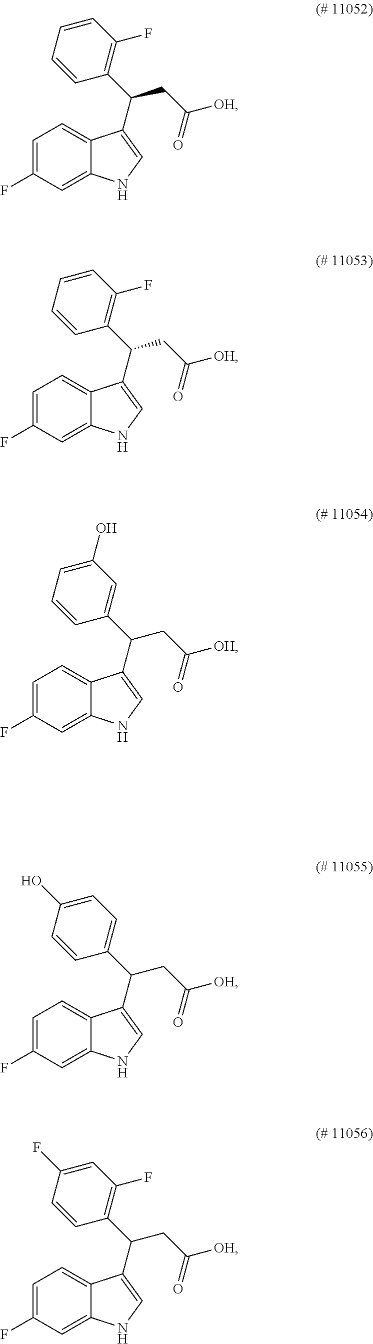 [SMILES: O=C(O)C[C@H](c1ccccc1F)c1c[nH]c2cc(F)ccc12 ; O=C(O)C[C@@H](c1ccccc1F)c1c[nH]c2cc(F)ccc12 ; O=C(O)CC(c1cccc(O)c1)c1c[nH]c2cc(F)ccc12 ; O=C(O)CC(c1ccc(O)cc1)c1c[nH]c2cc(F)ccc12 ; O=C(O)CC(c1ccc(F)cc1F)c1c[nH]c2cc(F)ccc12]

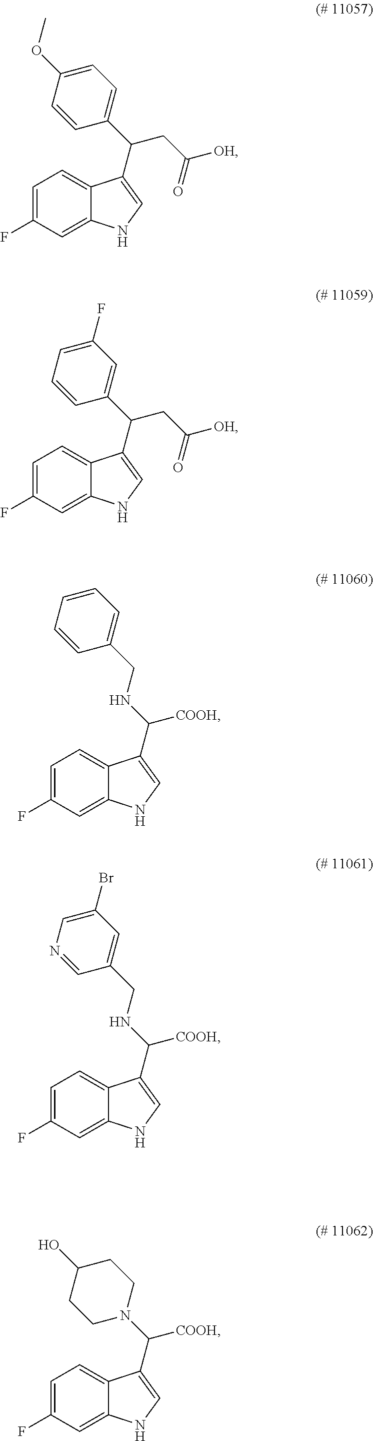 [SMILES: COc1ccc(C(CC(=O)O)c2c[nH]c3cc(F)ccc23)cc1 ; O=C(O)CC(c1cccc(F)c1)c1c[nH]c2cc(F)ccc12 ; O=C(O)C(NCc1ccccc1)c1c[nH]c2cc(F)ccc12 ; O=C(O)C(NCc1cncc(Br)c1)c1c[nH]c2cc(F)ccc12 ; O=C(O)C(c1c[nH]c2cc(F)ccc12)N1CCC(O)CC1]

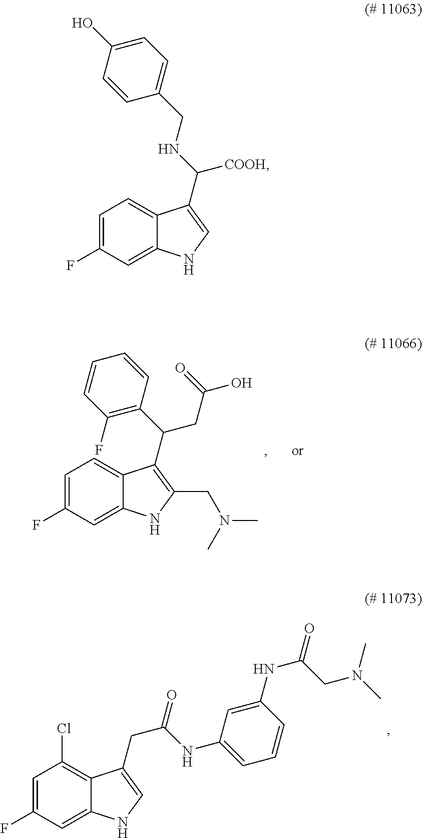 [SMILES: O=C(O)C(NCc1ccc(O)cc1)c1c[nH]c2cc(F)ccc12 ; CN(C)Cc1[nH]c2cc(F)ccc2c1C(CC(=O)O)c1ccccc1F ; CN(C)CC(=O)Nc1cccc(NC(=O)Cc2c[nH]c3cc(F)cc(Cl)c23)c1]

or a pharmaceutically acceptable salt, ester, solvate, isomer, or tautomer thereof.
40. The compound of embodiment 1 being Compound #10014, 10018, 10040, 10053, 10076, 10086, 10095, 10097, 10159, 11008, 11032, 11043, 11052, 11053, 11054, 11055, 11066, or 11073,
41. The compound of embodiment 1 being Compound #10014, 10095, 10097, 11008, 11043, 11052, 11053, 11054, 11055, 11066, or 11073.
42. The compound of embodiment 1 being Compound #10018, 10040, 10086, 10095, 10097, 10032, 11053, or 11066.
43. The compound of embodiment 1 being Compound #10095, 10097, 11032, 11053, or 11073.
44. The compound of embodiment 1 being Compound #11032, 11095, or 11097.
45. The compound of embodiment 1 being Compound #10018, 10040, 10086, or 11066.
46. The compound of any one of embodiments 1 to 12, wherein B)
   R¹⁰ represents —COOH,
   L represents a chain that is at most 3 atoms in length, and that is not —CH₂—CH(NH₂)—,
   any R¹¹ independently represents H, or
      alkyl, alkenyl, alkynyl, or alkenynyl, each of which independently unsubstituted or substituted with one or more R³⁰, and
   any R¹² independently represents H, —C≡N, —N(R¹⁵)₂, —C(═O)—R¹⁵, or
      alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰,
47. The compound of embodiment 46, wherein G represents —C(R⁴)═, preferably wherein R⁴ represents a halogen atom, preferably F.
48. The compound of embodiment 46 or 47, wherein L represents a chain that is at most 2 atoms in length, preferably only 1 atom in length.
49. The compound of any one of embodiments 46 to 48, wherein L represents —C(R¹²)₂— or —C(R¹²)₂—C(R¹²)₂—.
50. The compound of any one of embodiments 46 to 49, wherein all R¹² represent H.
51. The compound of embodiment any one of embodiments 46 to 49, wherein at least one (preferably one) R¹² represents:
   —C≡N, —N(R¹⁵)₂, —C(═O)—R¹⁵, or
   alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰,
   preferably —N(R¹⁵)₂, —C(═O)—R¹⁵, or
   cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰,
   more preferably —N(R¹⁵)₂, —C(═O)—R¹⁵, or
   heterocycloalkyl, heterocycloalkenyl, heterocycloalkynyl, heterocycloalkenynyl, aryl, or heteroaryl, each of which independently unsubstituted or substituted with one or more R³⁰, or
   yet more preferably —N(R¹⁵)₂, —C(═O)—R¹⁵, or
   heterocycloalkyl or aryl, each of which independently unsubstituted or substituted with one or more R³⁰,
   and preferably any other R¹² represent H.
52. The compound of any one of embodiments 46 to 51, wherein each R³⁰ independently is a halogen atom, —OR¹⁵, or
   alkyl, alkenyl, alkynyl, alkenynyl, each of which independently unsubstituted or substituted with one or more R¹⁶, preferably unsubstituted,
   preferably a halogen atom, —OR¹⁵, or alkyl unsubstituted or substituted with one or more R¹⁶, preferably unsubstituted.
53. The compound of any one of embodiments 46 to 52, wherein R¹⁵ represents H or alkyl, alkenyl, alkynyl, or alkenynyl, each of which independently unsubstituted or substituted with one or more R¹⁶, preferably unsubstituted.
54. The compound of any one of embodiments 46 to 53, wherein R¹⁵ represents H or alkyl unsubstituted or substituted with one or more R¹⁶, preferably unsubstituted.
55. The compound of embodiment 1 being

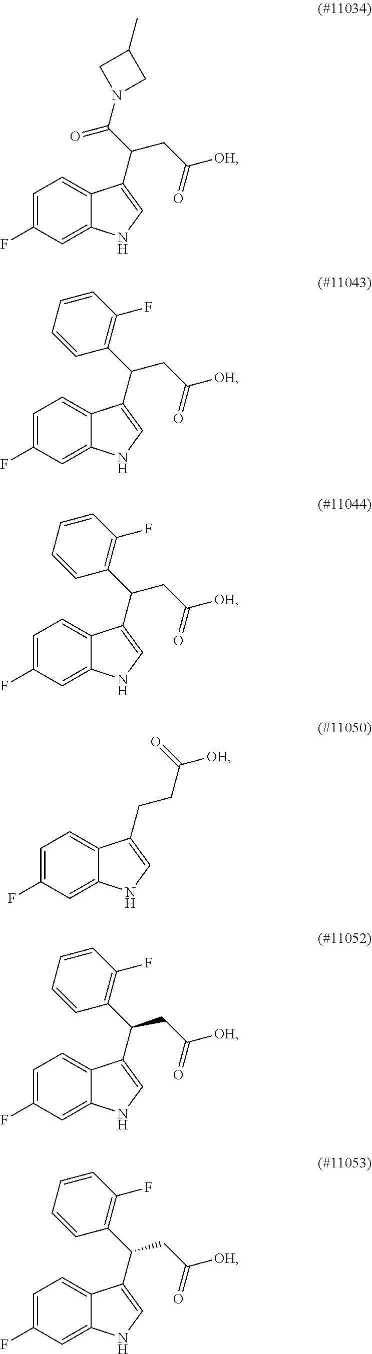 [SMILES: CC1CN(C(=O)C(CC(=O)O)c2c[nH]c3cc(F)ccc23)C1 ; O=C(O)CC(c1ccccc1F)c1c[nH]c2cc(F)ccc12 ; O=C(O)CC(c1ccccc1F)c1c[nH]c2cc(F)ccc12 ; O=C(O)CCc1c[nH]c2cc(F)ccc12 ; O=C(O)C[C@H](c1ccccc1F)c1c[nH]c2cc(F)ccc12 ; O=C(O)C[C@@H](c1ccccc1F)c1c[nH]c2cc(F)ccc12]

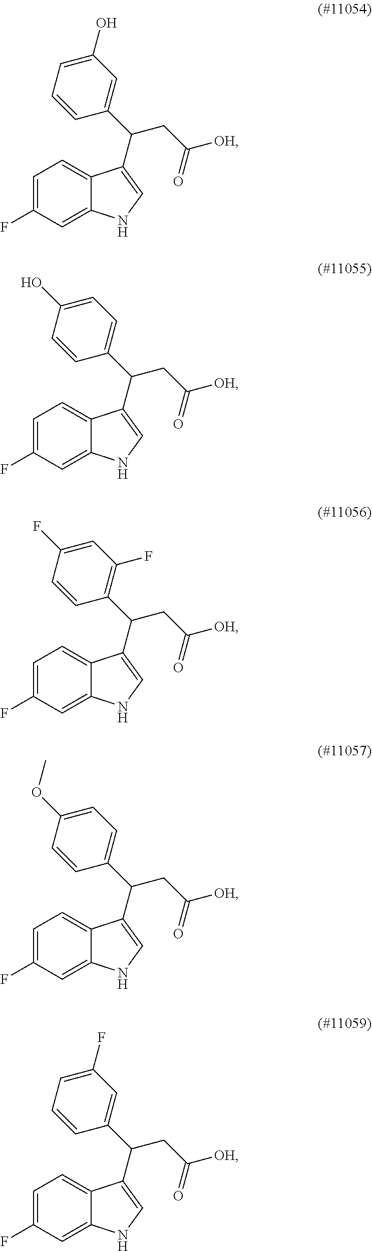 [SMILES: O=C(O)CC(c1cccc(O)c1)c1c[nH]c2cc(F)ccc12 ; O=C(O)CC(c1ccc(O)cc1)c1c[nH]c2cc(F)ccc12 ; O=C(O)CC(c1ccc(F)cc1F)c1c[nH]c2cc(F)ccc12 ; COc1ccc(C(CC(=O)O)c2c[nH]c3cc(F)ccc23)cc1 ; O=C(O)CC(c1cccc(F)c1)c1c[nH]c2cc(F)ccc12]

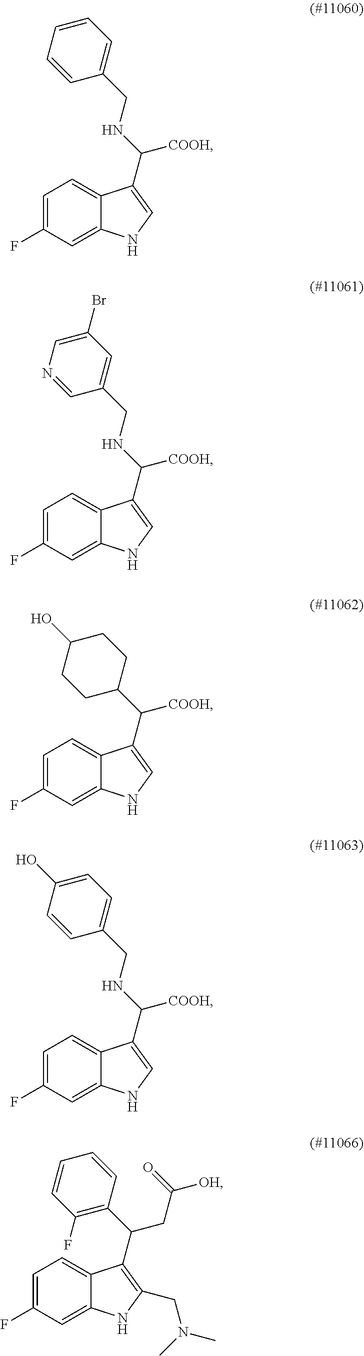 [SMILES: O=C(O)C(NCc1ccccc1)c1c[nH]c2cc(F)ccc12 ; O=C(O)C(NCc1cncc(Br)c1)c1c[nH]c2cc(F)ccc12 ; O=C(O)C(c1c[nH]c2cc(F)ccc12)C1CCC(O)CC1 ; O=C(O)C(NCc1ccc(O)cc1)c1c[nH]c2cc(F)ccc12 ; CN(C)Cc1[nH]c2cc(F)ccc2c1C(CC(=O)O)c1ccccc1F]

or a pharmaceutically acceptable salt, ester, solvate, isomer, or tautomer thereof.
56. The compound of embodiment 1, being compound #11066.
57. The compound of any one of embodiments 1 to 12, wherein C)
   R¹⁰ is attached to a nitrogen atom of a —N(R¹¹)— group that ends the chain in L, and R¹⁰ and the R¹¹ of the —N(R¹¹)— group that ends the chain in L together with the nitrogen atom to which they are attached form a heteroaryl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl, each of which independently unsubstituted or substituted with:
      a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR²⁰, —OR¹⁵, —O—C(═O)—R¹⁵, —SO₂—R¹⁵, —SO—R¹⁵, —N(R¹⁵)₂—SO₂—R¹⁵, —N(R¹⁵)₂—SO—R¹⁵, or
      alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R²¹,
      with the proviso that when R¹⁰ and R¹¹ together with the nitrogen atom to which they are attached form morpholinyl, the morpholinyl is free of a substituent containing a —C(═O)— group,
      with the proviso that when R¹⁰ and R¹¹ together with the nitrogen atom to which they are attached form piperazinyl substituted with a —C(═O)—O—CH₃ group, the —C(═O)—O—CH₃ group is in position 2,
      with the proviso that when R¹⁰ and R¹¹ together with the nitrogen atom to which they are attached form piperazinyl N-substituted with —O-heterocycloalkyl, the heterocycloalkyl is other than oxalanyl,
   the chain in L is at most 4 atoms in length and ends with the —N(R¹¹)— group to which R¹⁰ is attached,
      with the proviso that -L-R¹⁰ is not

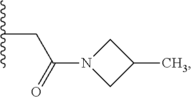 [SMILES: *CC(=O)N1CC(C)C1]

any other R¹¹ independently represents H, or
   alkyl, alkenyl, alkynyl, or alkenynyl, each of which independently unsubstituted or substituted with one or more R³¹ and
any R¹² independently represents H, —C≡N, —N(R¹⁵)₂, or
   alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, eterocycloalkenynonyl, aryl, or heteroaryl, each of which independently unsubstituted or substituted with one or more R³¹,
   with the proviso that when one R¹² on a given carbon atom is methyl, any other R¹² on said given carbon atom is other than methyl.
58. The compound of embodiment 57, wherein L comprises at least one —C(═O)— and a least one-N(R¹¹)—.
59. The compound of embodiment 57 or 58, wherein L comprises exactly one —C(═O)— and exactly one-N(R¹¹)—.
60. The compound of any one of embodiments 57 to 59, wherein -L- ends with a —C(═O)—N(R¹¹)— group.
61. The compound of any one of embodiments 57 to 60, wherein L represents —C(R¹²)₂—C(═O)—N(R¹¹)— or —C(═O)—C(R¹²)₂—N(R¹¹)—, preferably —C(R¹²)₂—C(═O)—N(R¹¹)—.
62. The compound of any one of embodiments 57 to 61, wherein each R¹² independently represents H, —C≡N, or alkyl (preferably methyl or ethyl).
63. The compound of any one of embodiments 57 to 62, wherein a first R¹² on a carbon atom represents alkyl (preferably methyl or ethyl), or —C≡N, and a second R¹² on said carbon atom represent H.
64. The compound of any one of embodiments 57 to 62, wherein two R¹² on a carbon atom represent alkyl.
65. The compound of any one of embodiments 57 to 62, wherein two R¹² on a carbon atom represents H.
66. The compound of any one of embodiments 57 to 62, wherein any other R¹¹ represents H.
67. The compound of any one of embodiments 57 to 66, wherein R¹⁰ and the R¹¹ together with the nitrogen atom to which they are attached form a heterocycloalkyl, preferably azetidin-1-yl, pyrrolidi-1-nyl, piperidin-1-yl, piperidinon-1-yl (preferably piperidin-3-on-1-yl or piperidin-4-on-1-yl), morpholin-1-yl, piperazin-1-yl, piperazinone-1-yl (more preferably piperazin-3-on-1-yl),

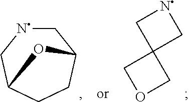

most preferably azetidin-1-yl, piperidin-1-yl, or morpholin-1-yl.
68. The compound of any one of embodiments 57 to 67, wherein the heteroaryl, heterocycloalkyl, heterocycloalkenyl, heterocycloalkynyl and heterocycloalkenynyl formed by R¹⁰ and R¹¹ together with the nitrogen atom to which they are attached is unsubstituted or substituted with one or more (preferably one or two, more preferably one) of alkyl, —OR¹⁵, —C(═O)—N(R¹⁵)₂, —N(R¹⁵)—C(═O)—R¹⁵, or —C(═O)—OR²⁰, more preferably unsubstituted or substituted with one or more (preferably one) of:
   alkyl (preferably C₁₋₆, more preferably methyl or ethyl),
   OH,
   alkoxy (preferably C₁₋₆, more preferably methoxy, ethoxy, propoxy (preferably isopropoxy), or
   butoxy (preferably isobutoxy)),
   —CONH₂,
   ═NH—C(═O)-alkyl (preferably C₁₋₆, more preferably ═NH—C(═O)—CH₃),
   —C(═O)O-alkyl (preferably C₁₋₆, more preferably —C(═O)O—CH₂CH₃),
   and most preferably unsubstituted or substituted with one or more, preferably one or two, more preferably one of alkyl, hydroxyl, or —CH₂—N(CH₃)₂.
69. The compound of any one of embodiments 57 to 68, wherein the R¹⁵ of the OR¹⁵ group, the alkoxy and/or the alkyl substituting the cycle formed by R¹⁰ and R¹¹ are unsubstituted or substituted with one or more of alkoxy (preferably C₁₋₆, more preferably methoxy), alkoxycarbonyl (preferably C₁₋₆, more preferably —C(═O)OCH₂CH₃), hydroxyl, amino, alkylamino, or dialkylamino (preferably C₁₋₆, more preferably —N(CH₃)₂).
70. The compound of embodiment 1 being:
71.

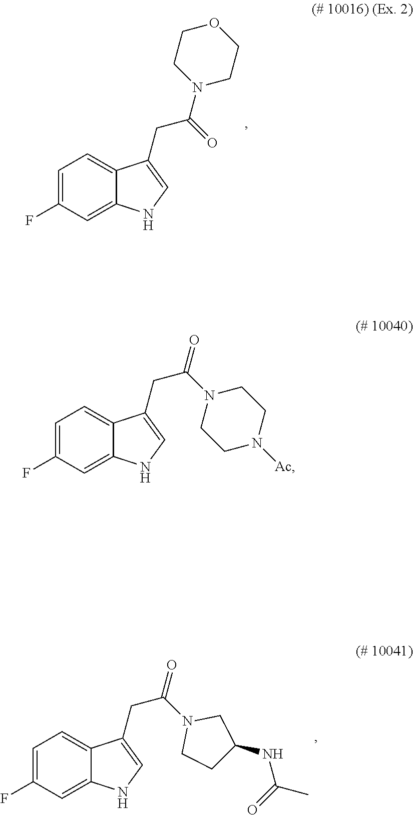 [SMILES: O=C(Cc1c[nH]c2cc(F)ccc12)N1CCOCC1 ; CC(=O)N1CCN(C(=O)Cc2c[nH]c3cc(F)ccc23)CC1 ; CC(=O)N[C@H]1CCN(C(=O)Cc2c[nH]c3cc(F)ccc23)C1]

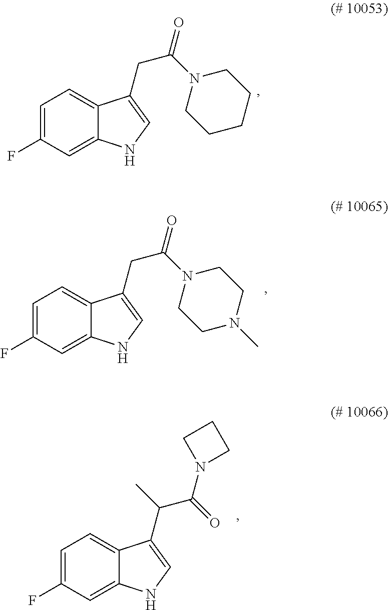 [SMILES: O=C(Cc1c[nH]c2cc(F)ccc12)N1CCCCC1 ; CN1CCN(C(=O)Cc2c[nH]c3cc(F)ccc23)CC1 ; CC(C(=O)N1CCC1)c1c[nH]c2cc(F)ccc12]

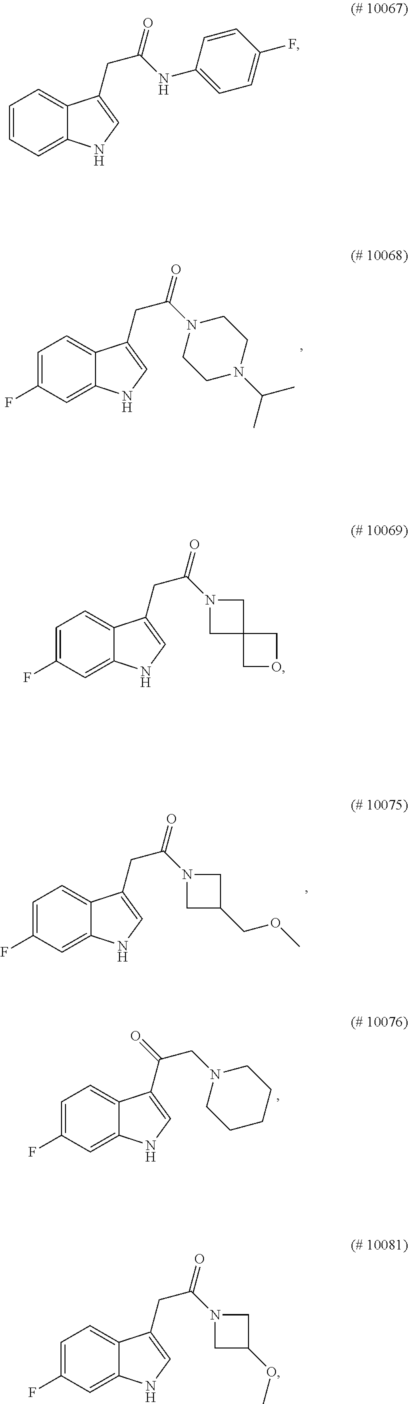 [SMILES: O=C(Cc1c[nH]c2ccccc12)Nc1ccc(F)cc1 ; CC(C)N1CCN(C(=O)Cc2c[nH]c3cc(F)ccc23)CC1 ; O=C(Cc1c[nH]c2cc(F)ccc12)N1CC2(COC2)C1 ; COCC1CN(C(=O)Cc2c[nH]c3cc(F)ccc23)C1 ; O=C(CN1CCCCC1)c1c[nH]c2cc(F)ccc12 ; COC1CN(C(=O)Cc2c[nH]c3cc(F)ccc23)C1]

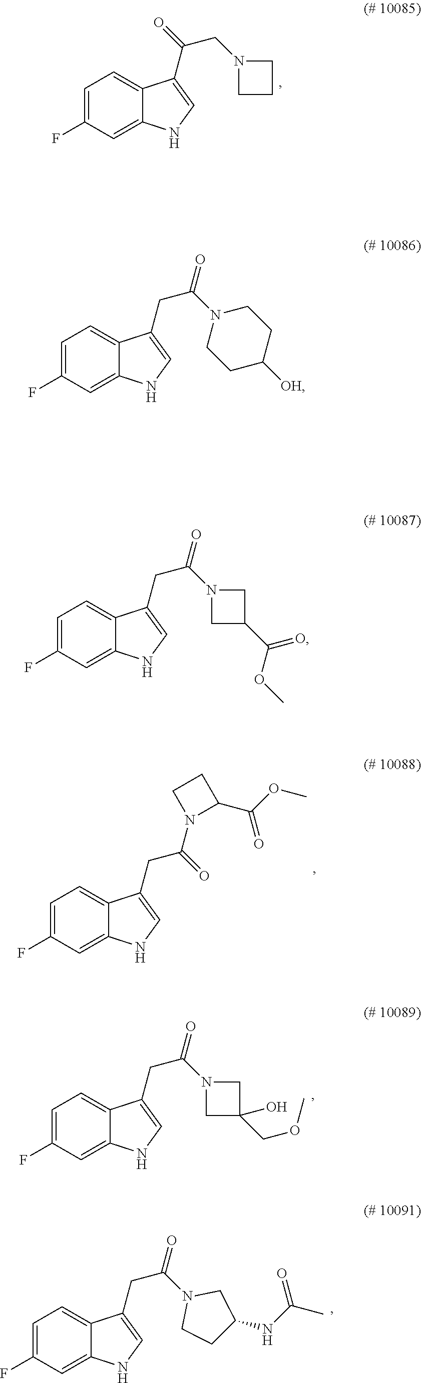 [SMILES: O=C(CN1CCC1)c1c[nH]c2cc(F)ccc12 ; O=C(Cc1c[nH]c2cc(F)ccc12)N1CCC(O)CC1 ; COC(=O)C1CN(C(=O)Cc2c[nH]c3cc(F)ccc23)C1 ; COC(=O)C1CCN1C(=O)Cc1c[nH]c2cc(F)ccc12 ; COCC1(O)CN(C(=O)Cc2c[nH]c3cc(F)ccc23)C1 ; CC(=O)N[C@@H]1CCN(C(=O)Cc2c[nH]c3cc(F)ccc23)C1]

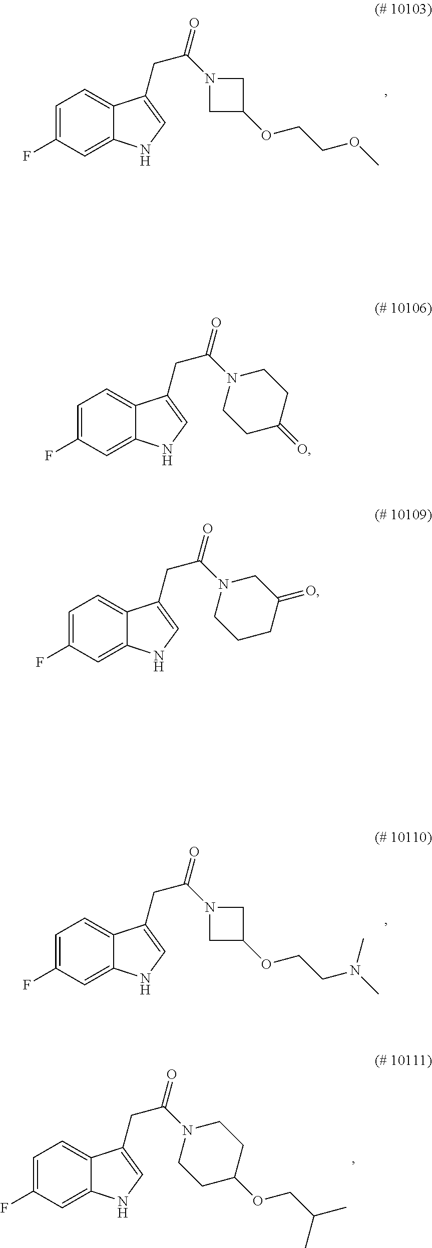 [SMILES: COCCOC1CN(C(=O)Cc2c[nH]c3cc(F)ccc23)C1 ; O=C1CCN(C(=O)Cc2c[nH]c3cc(F)ccc23)CC1 ; O=C1CCCN(C(=O)Cc2c[nH]c3cc(F)ccc23)C1 ; CN(C)CCOC1CN(C(=O)Cc2c[nH]c3cc(F)ccc23)C1 ; CC(C)COC1CCN(C(=O)Cc2c[nH]c3cc(F)ccc23)CC1]

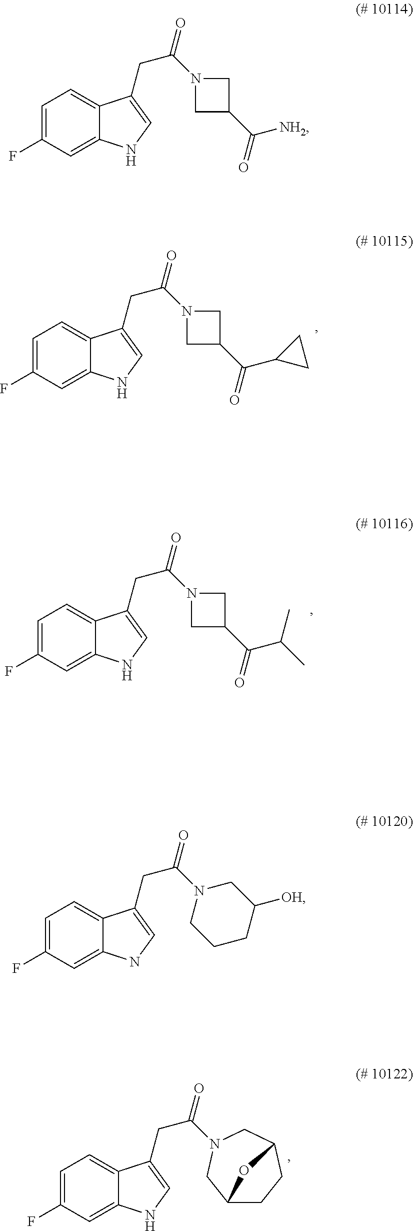 [SMILES: NC(=O)C1CN(C(=O)Cc2c[nH]c3cc(F)ccc23)C1 ; O=C(C1CC1)C1CN(C(=O)Cc2c[nH]c3cc(F)ccc23)C1 ; CC(C)C(=O)C1CN(C(=O)Cc2c[nH]c3cc(F)ccc23)C1 ; O=C(Cc1c[nH]c2cc(F)ccc12)N1CCCC(O)C1 ; O=C(Cc1c[nH]c2cc(F)ccc12)N1C[C@H]2CC[C@@H](C1)O2]

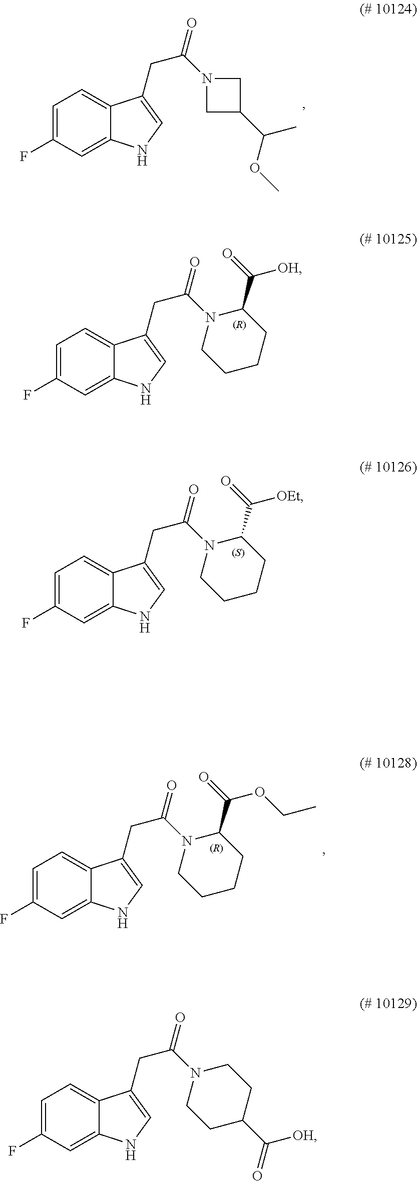

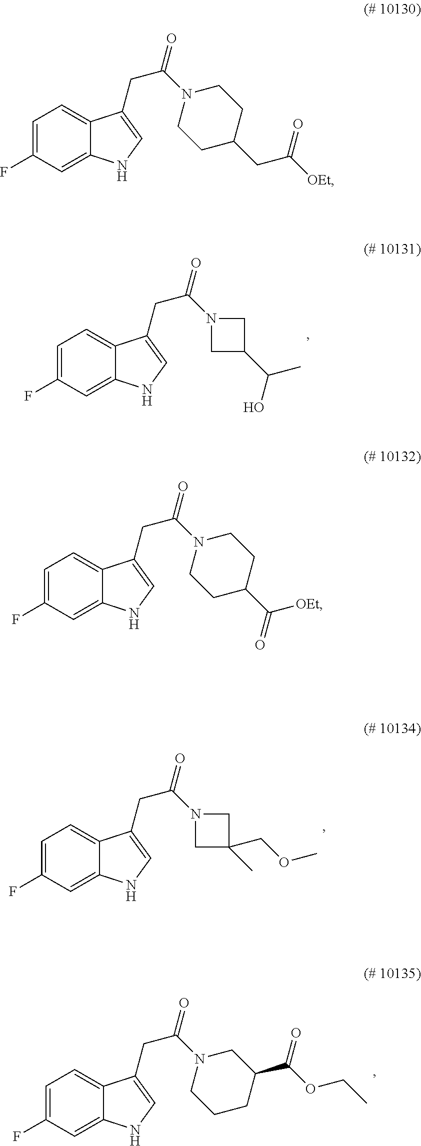 [SMILES: CCOC(=O)CC1CCN(C(=O)Cc2c[nH]c3cc(F)ccc23)CC1 ; CC(O)C1CN(C(=O)Cc2c[nH]c3cc(F)ccc23)C1 ; CCOC(=O)C1CCN(C(=O)Cc2c[nH]c3cc(F)ccc23)CC1 ; COCC1(C)CN(C(=O)Cc2c[nH]c3cc(F)ccc23)C1 ; CCOC(=O)[C@H]1CCCN(C(=O)Cc2c[nH]c3cc(F)ccc23)C1]

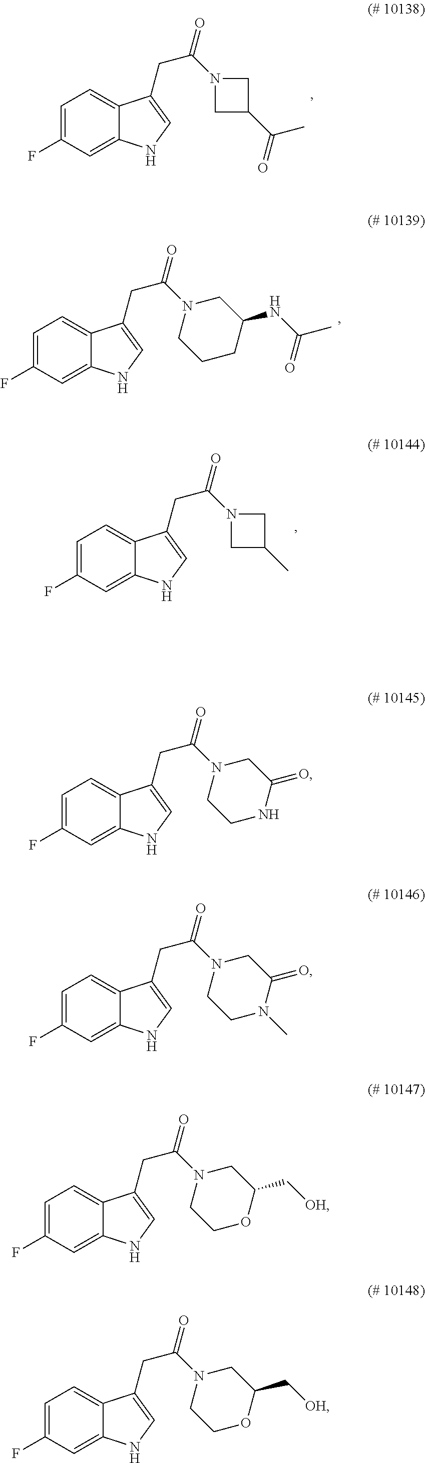

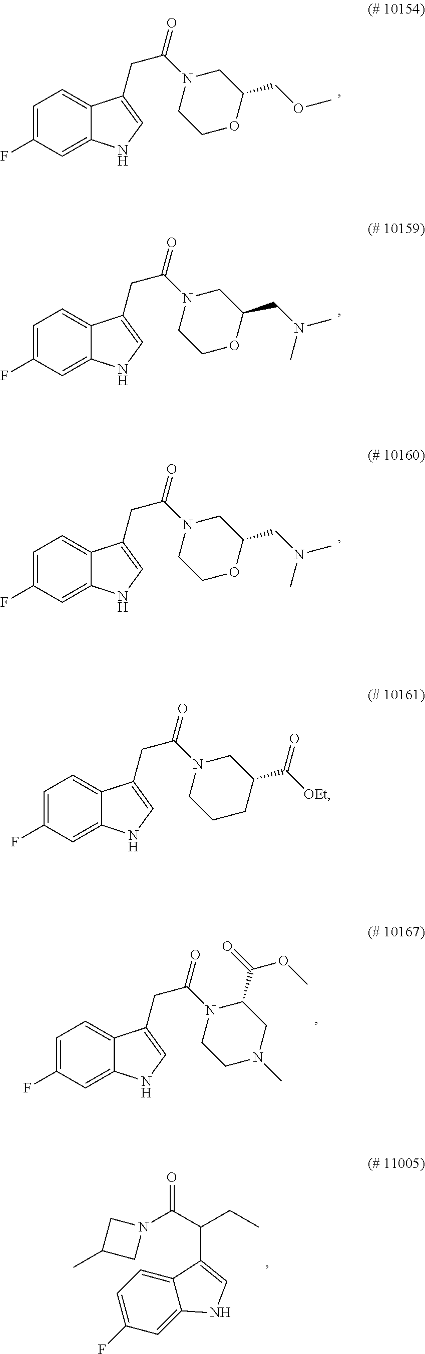

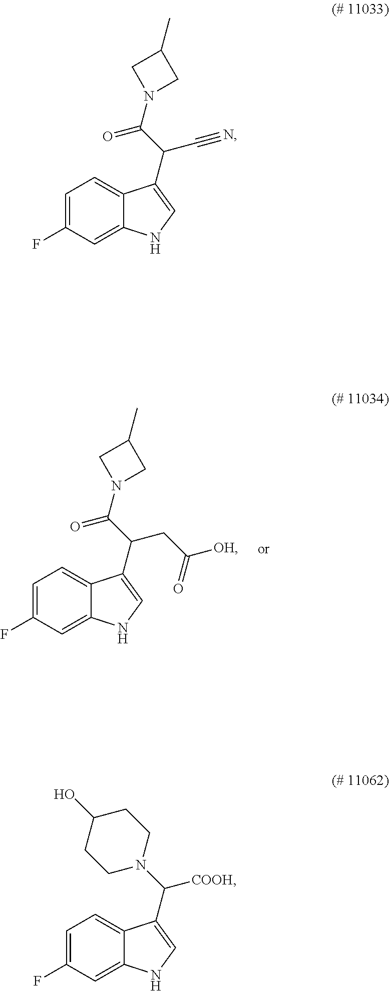 [SMILES: CC1CN(C(=O)C(C#N)c2c[nH]c3cc(F)ccc23)C1 ; CC1CN(C(=O)C(CC(=O)O)c2c[nH]c3cc(F)ccc23)C1 ; O=C(O)C(c1c[nH]c2cc(F)ccc12)N1CCC(O)CC1]

or a pharmaceutically acceptable salt, ester, solvate, isomer, or tautomer thereof.
72. The compound of embodiment 1 being compound #10040, 10053, 10066, 10076, 10086, 10159, and 11005. Most preferred compound include Compound #10066 or 11005.
73. The compound of any one of embodiments 1 to 12, wherein D)
   R¹⁰ represents H, a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR¹⁵, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵, or
      alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each independently unsubstituted or substituted with one or more R¹⁶,
      the chain in L is at most 3 atoms in length, contains at least one —C(R¹²)₂— group, and ends with a —N(R¹¹)— group,
      the R¹¹ of the —N(R¹¹)— group that ends the chain in L and one R¹² of said at least one —C(R¹²)₂— group together with the one or more atoms to which they are attached and any atom(s) between said one or more atoms form heterocycloalkyl, heterocycloalkanonyl, heterocycloalkenonyl, heterocycloalkynonyl, heterocycloalkenynonyl, or heteroaryl, each of which being independently unsubstituted or substituted with:
         a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR¹⁵, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵,
         alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which being independently unsubstituted or substituted with one or more R¹⁶,
         with the proviso that when said one R¹¹ and said one R¹² form pyrrolidinyl, the pyrrolidinyl is substituted,
         with the proviso that when the heterocycloalkyl is piperidinyl, R¹⁰ is other than 2,4-difluorophenylcarbonyl substituent,
      any other R¹¹ independently represent H or
         alkyl, alkenyl, alkynyl, alkenynyl, each of which independently unsubstituted or substituted with one or more R³¹, and
      any other R¹² independently represent H, —C≡N, or —N(R¹⁵)₂,
         alkyl, alkenyl, alkynyl, or alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³¹,
         with the proviso that -L-R¹⁰ is not

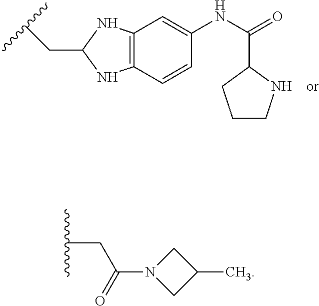 [SMILES: *CC1Nc2ccc(NC(=O)C3CCCN3)cc2N1 ; *CC(=O)N1CC(C)C1]

74. The compound of embodiment 73, wherein L represents:
   —(C(R¹²)₂—)ₙ—C(═O)—N(R¹¹)—, wherein n is 1 or 2, or
   —(C(R¹²)₂—)ₙ—N(R¹¹)—, wherein n is 2 or 3,
   wherein the R¹¹ and one of the R¹² of these groups together with the two atoms to which they are attached and any atom between said two atoms form the heteroaryl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl, each of which being independently unsubstituted or substituted as described in embodiment 1.
75. The compound of embodiment 73, wherein L represents:
   —C*(R¹²)₂—C(═O)—N(R¹¹)—,
   —C*(R¹²)₂—C(R¹²)₂—C(R¹²)₂—N(R¹¹)—, or
   —C*(R¹²)₂—C(R¹²)₂—N(R¹)—
   wherein one of the R¹¹ and one of the R¹² (preferably one of the R¹² attached to the carbon atom indicated with a *) together with the two atoms to which they are attached and any atom between said two atoms form the heteroaryl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl, each of which being independently unsubstituted or substituted as described in embodiment 1.
76. The compound of any one of embodiments 72 to 75, wherein the R¹² that form the heteroaryl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl is attached to the carbon atom in L closest to G, J, X or Q, i.e. the carbon atoms indicated by a star.
77. The compound of any one of embodiments 72 to 76, wherein the R¹¹ and the R¹² form a heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl,
   preferably a heterocycloalkyl or a heterocycloalkanonyl,
   more preferably pyrrolidinyl (preferably pyrrolidin-3-yl), succinimidyl, (preferably succinimid-3-yl), or piperidinyl (preferably piperidin-4-yl).
78. The compound of any one of embodiments 72 to 77, wherein the R¹¹ and the R¹² form pyrrolidinyl (preferably pyrrolidin-3-yl).
79. The compound of any one of embodiments 72 to 77, wherein the R¹¹ and the R¹² form succinimidyl (preferably succinimid-3-yl).
80. The compound of any one of embodiments 72 to 77, wherein the R¹¹ and the R¹² form piperidinyl (preferably piperidin-4-yl).
81. The compound of any one of embodiments 72 to 80, wherein any other R¹¹ represents H.
82. The compound of any one of embodiments 72 to 81, wherein any other R¹² represents H.
83. The compound of any one of embodiments 72 to 82, wherein R¹⁰ represents H or —C(═O)—R¹⁵, preferably —C(═O)—R¹⁵, more preferably R¹⁰ represents H or —C(═O)—R²⁰, most preferably —C(═O)—R²⁰.
84. The compound of any one of embodiments 72 to 83, wherein, R¹⁵ (or R²⁰ as the case may be) represents one of the following groups:
   alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, aryl, or heteroaryl,
   preferably cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, aryl, or heteroaryl, each of which preferably being 5- or 6-membered, more preferably cycloalkyl, cycloalkenyl, cycloalkynyl, cycloalkenynyl, heterocycloalkyl, heterocycloalkenyl, heterocycloalkynyl, heterocycloalkenynyl, or aryl, each of which preferably being 5- or 6-membered, yet more preferably cycloalkyl, heterocycloalkyl, or aryl, each of which preferably being 5- or 6-membered, each of which independently unsubstituted or substituted with one or more R¹⁶. Preferred such cycloalkyls include cyclohexyl. Preferred such heterocycloalkyls include piperidinyl (preferably piperidin-4-yl or piperidin-3-yl) and pyrrolidinyl (preferably pyrrolidin-3-yl). Preferred such aryls include phenyl.
85. The compound of any one of embodiments 72 to 84, wherein the group in R¹⁵ is unsubstituted.
86. The compound of any one of embodiments 72 to 85, wherein the group in R¹⁵ is heterocycloalkyl, heterocycloalkenyl, heterocycloalkynyl, heterocycloalkenynyl, or heteroaryl, R¹⁶ is preferably substituting a heteroatom of these groups (preferably N).
87. The compound of any one of embodiments 72 to 86, wherein R¹⁶ is:
   a halogen atom, alkylcarbonyl, alkenylcarbonyl, alkynylcarbonyl, alkenynylcarbonyl, alkoxycarbonyl, alkylcarbamoyl, alkenylcarbamoyl, alkynylcarbamoyl, alkenynylcarbamoyl, or hydroxyl, preferably a halogen atom, alkylcarbonyl, alkoxycarbonyl, alkylcarbamoyl, or hydroxyl, more preferably a halogen atom, alkylcarbonyl, alkylcarbamoyl, or hydroxyl.
88. The compound of any one of embodiments 72 to 87, wherein when R¹⁶ is substituting a nitrogen atom, R¹⁶ is alkylcarbonyl or alkoxycarbonyl, preferably alkylcarbonyl.
89. The compound of embodiment 1 being:
90.

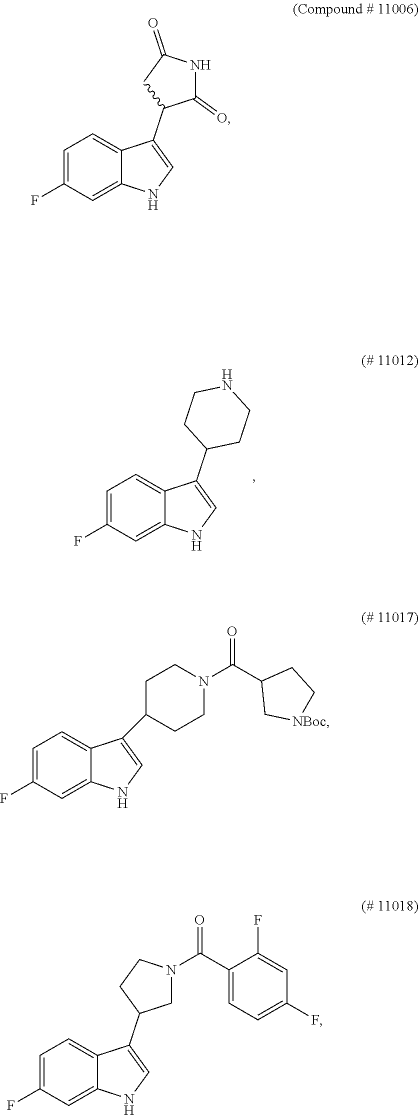 [SMILES: O=C1CC(c2c[nH]c3cc(F)ccc23)C(=O)N1 ; Fc1ccc2c(C3CCNCC3)c[nH]c2c1 ; CC(C)(C)OC(=O)N1CCC(C(=O)N2CCC(c3c[nH]c4cc(F)ccc34)CC2)C1 ; O=C(c1ccc(F)cc1F)N1CCC(c2c[nH]c3cc(F)ccc23)C1]

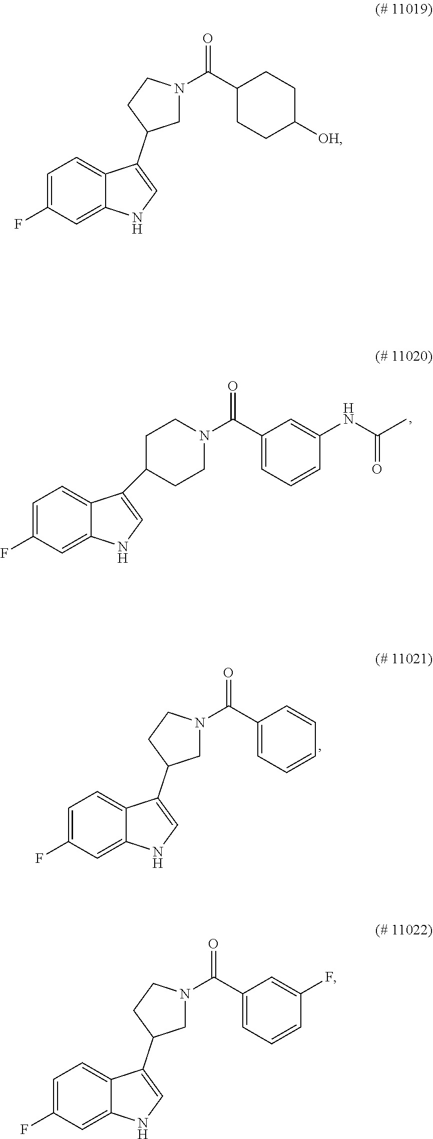 [SMILES: O=C(C1CCC(O)CC1)N1CCC(c2c[nH]c3cc(F)ccc23)C1 ; CC(=O)Nc1cccc(C(=O)N2CCC(c3c[nH]c4cc(F)ccc34)CC2)c1 ; O=C(c1ccccc1)N1CCC(c2c[nH]c3cc(F)ccc23)C1 ; O=C(c1cccc(F)c1)N1CCC(c2c[nH]c3cc(F)ccc23)C1]

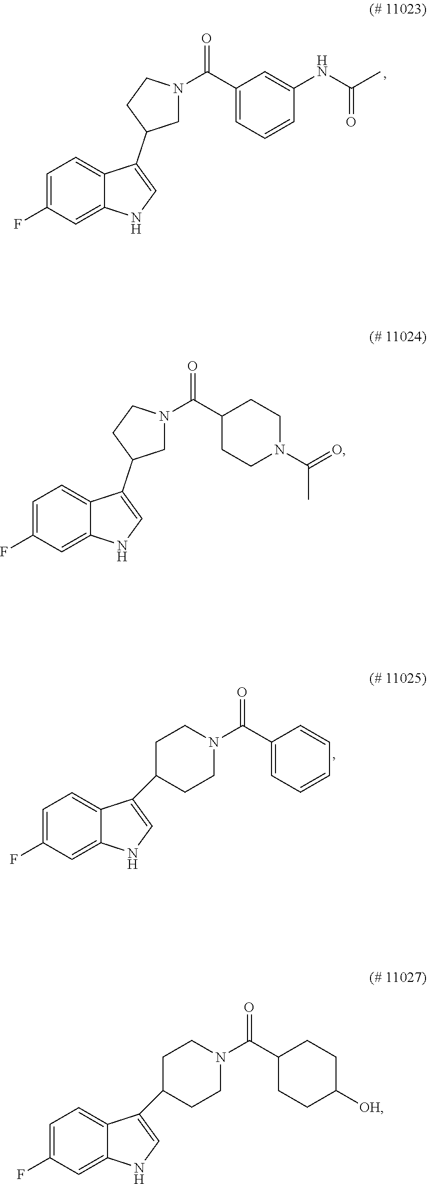 [SMILES: CC(=O)Nc1cccc(C(=O)N2CCC(c3c[nH]c4cc(F)ccc34)C2)c1 ; CC(=O)N1CCC(C(=O)N2CCC(c3c[nH]c4cc(F)ccc34)C2)CC1 ; O=C(c1ccccc1)N1CCC(c2c[nH]c3cc(F)ccc23)CC1 ; O=C(C1CCC(O)CC1)N1CCC(c2c[nH]c3cc(F)ccc23)CC1]

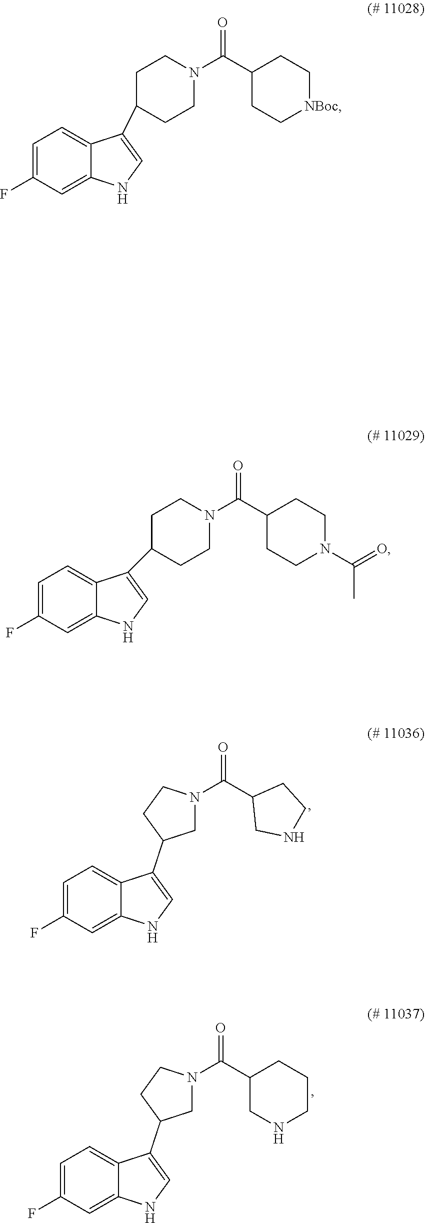 [SMILES: CC(C)(C)OC(=O)N1CCC(C(=O)N2CCC(c3c[nH]c4cc(F)ccc34)CC2)CC1 ; CC(=O)N1CCC(C(=O)N2CCC(c3c[nH]c4cc(F)ccc34)CC2)CC1 ; O=C(C1CCNC1)N1CCC(c2c[nH]c3cc(F)ccc23)C1 ; O=C(C1CCCNC1)N1CCC(c2c[nH]c3cc(F)ccc23)C1]

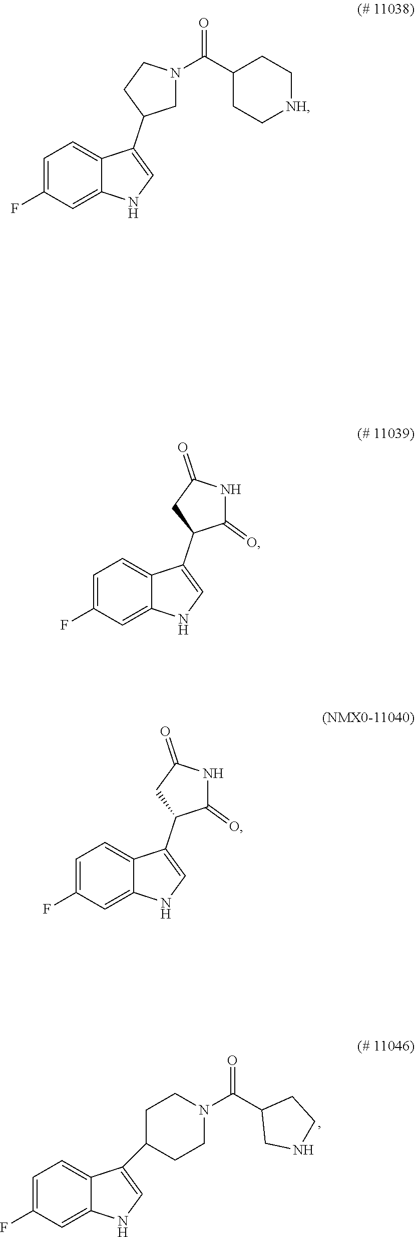 [SMILES: O=C(C1CCNCC1)N1CCC(c2c[nH]c3cc(F)ccc23)C1 ; O=C1C[C@H](c2c[nH]c3cc(F)ccc23)C(=O)N1 ; O=C1C[C@@H](c2c[nH]c3cc(F)ccc23)C(=O)N1 ; O=C(C1CCNC1)N1CCC(c2c[nH]c3cc(F)ccc23)CC1]

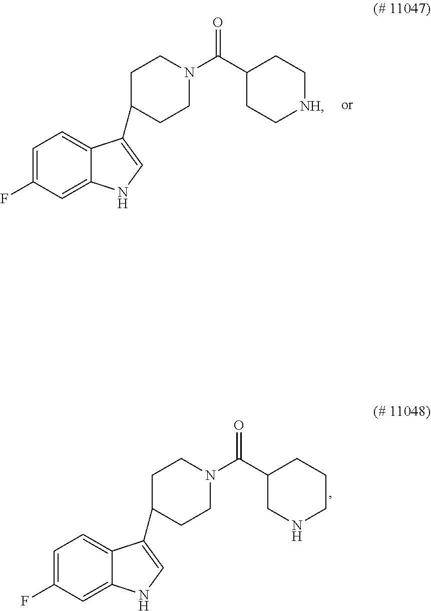 [SMILES: O=C(C1CCNCC1)N1CCC(c2c[nH]c3cc(F)ccc23)CC1 ; "or" ; O=C(C1CCCNC1)N1CCC(c2c[nH]c3cc(F)ccc23)CC1]

or a pharmaceutically acceptable salt, ester, solvate, isomer, or tautomer thereof.
91. The compound of embodiment 1 being compound #11006.
92. The compound of any one of embodiments 1 to 91 being of formula (II):

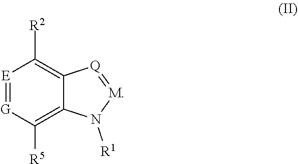

93. The compound of embodiment 92, wherein:
   E represents —CH═,
   G represents —C(R⁴)═, preferably wherein R⁴ represents a halogen atom, more preferably F,
   M represents —N═ or —C(R⁷)═,
   Q represents —C(R⁸)═, preferably —C(L-R¹⁰)═, wherein L and R¹⁰ are as defined in any one of embodiments 1 to 91,
   R¹ represents H,
   R² represents H, and/or
   R⁴ represents H, and
   with the proviso that the compound of formula (II) comprises exactly one -L-R¹⁰ group,
   or a pharmaceutically acceptable salt, ester, solvate, isomer, or tautomer thereof.
94. The compound of embodiment 92, wherein M represents —C(R⁷)═, preferably —CH═.
95. The compound of embodiment 92, wherein, M represents —N═.
96. The compound of embodiment 92 being of formula (III):

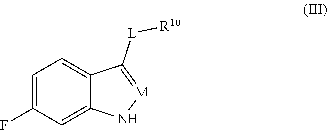

or a pharmaceutically acceptable salt, ester, solvate, isomer, or tautomer thereof.
97. The compound of embodiment 96, wherein M represents —C(R⁷)═, preferably —CH═.
98. The compound of embodiment 96, wherein M represents —N═.
99. The compound of embodiment 96 being of formula (IV):

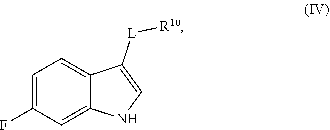

or a pharmaceutically acceptable salt, ester, solvate, isomer, or tautomer thereof.
100. The compound of embodiment 1 being:
101.

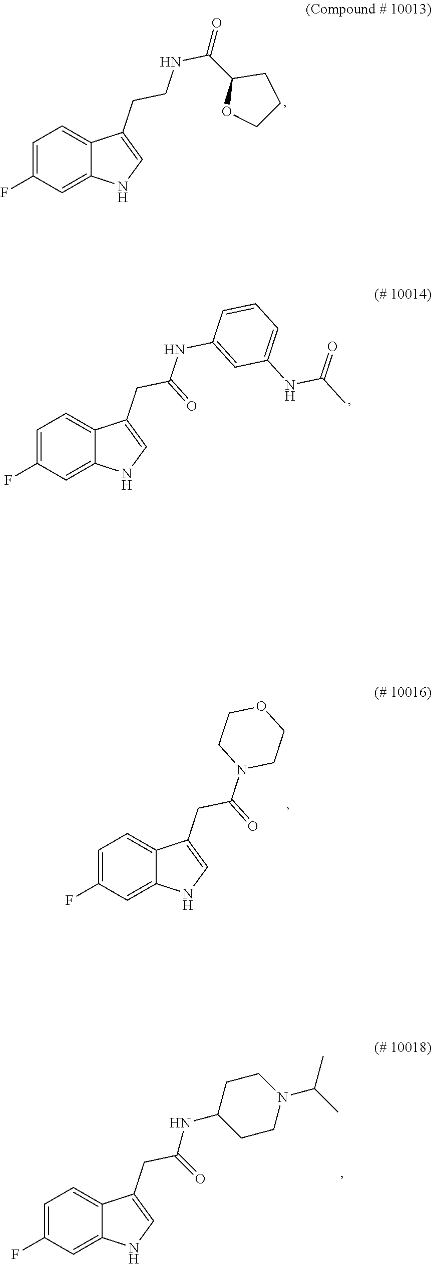 [SMILES: O=C(NCCc1c[nH]c2cc(F)ccc12)[C@H]1CCCO1 ; CC(=O)Nc1cccc(NC(=O)Cc2c[nH]c3cc(F)ccc23)c1 ; O=C(Cc1c[nH]c2cc(F)ccc12)N1CCOCC1 ; CC(C)N1CCC(NC(=O)Cc2c[nH]c3cc(F)ccc23)CC1]

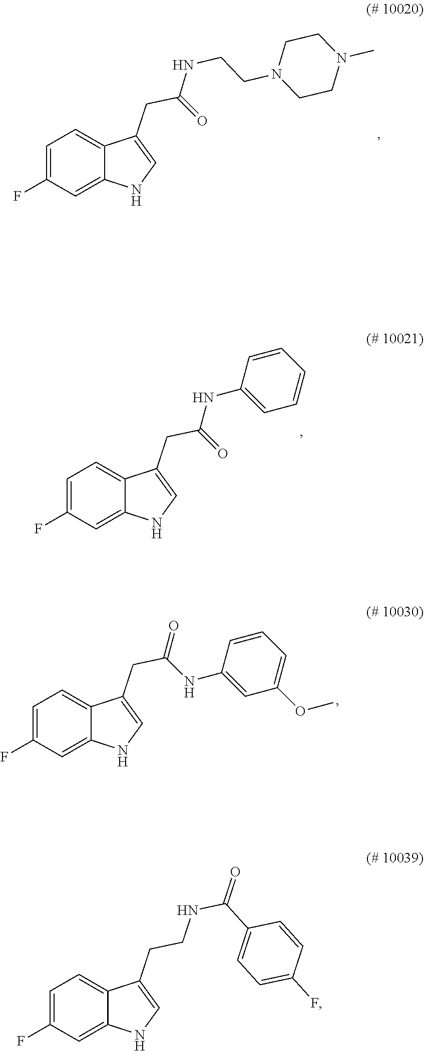 [SMILES: CN1CCN(CCNC(=O)Cc2c[nH]c3cc(F)ccc23)CC1 ; O=C(Cc1c[nH]c2cc(F)ccc12)Nc1ccccc1 ; COc1cccc(NC(=O)Cc2c[nH]c3cc(F)ccc23)c1 ; O=C(NCCc1c[nH]c2cc(F)ccc12)c1ccc(F)cc1]

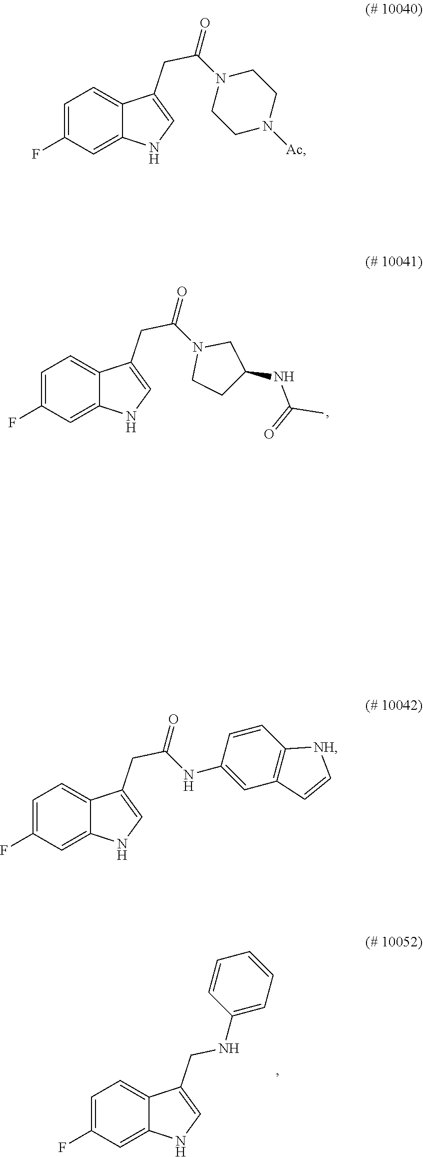 [SMILES: CC(=O)N1CCN(C(=O)Cc2c[nH]c3cc(F)ccc23)CC1 ; CC(=O)N[C@H]1CCN(C(=O)Cc2c[nH]c3cc(F)ccc23)C1 ; O=C(Cc1c[nH]c2cc(F)ccc12)Nc1ccc2[nH]ccc2c1 ; Fc1ccc2c(CNc3ccccc3)c[nH]c2c1]

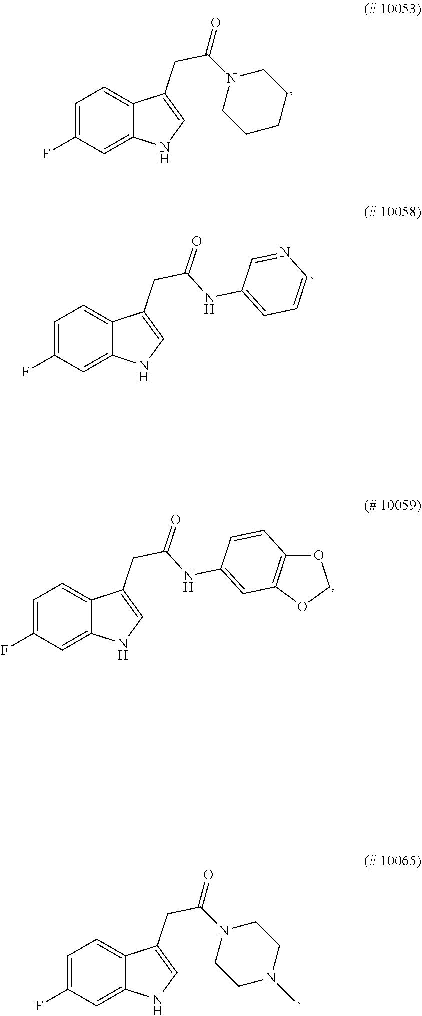 [SMILES: O=C(Cc1c[nH]c2cc(F)ccc12)N1CCCCC1 ; O=C(Cc1c[nH]c2cc(F)ccc12)Nc1cccnc1 ; O=C(Cc1c[nH]c2cc(F)ccc12)Nc1ccc2c(c1)OCO2 ; CN1CCN(C(=O)Cc2c[nH]c3cc(F)ccc23)CC1]

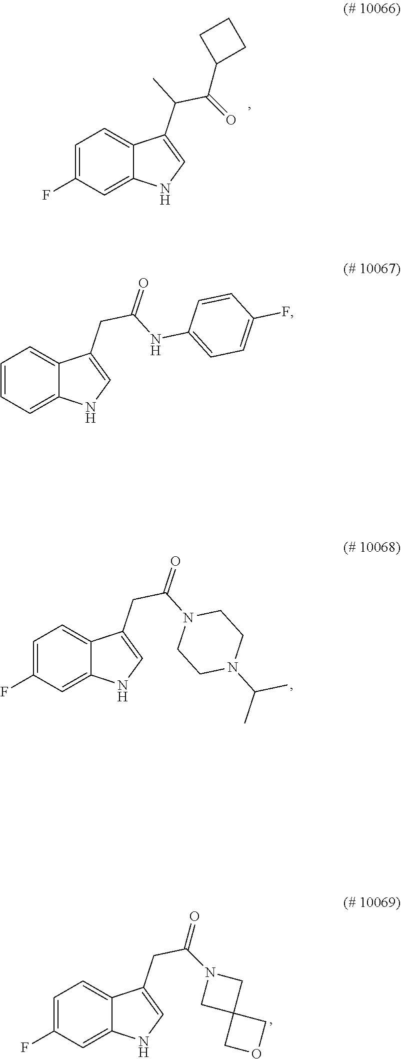 [SMILES: CC(C(=O)C1CCC1)c1c[nH]c2cc(F)ccc12 ; O=C(Cc1c[nH]c2ccccc12)Nc1ccc(F)cc1 ; CC(C)N1CCN(C(=O)Cc2c[nH]c3cc(F)ccc23)CC1 ; O=C(Cc1c[nH]c2cc(F)ccc12)N1CC2(COC2)C1]

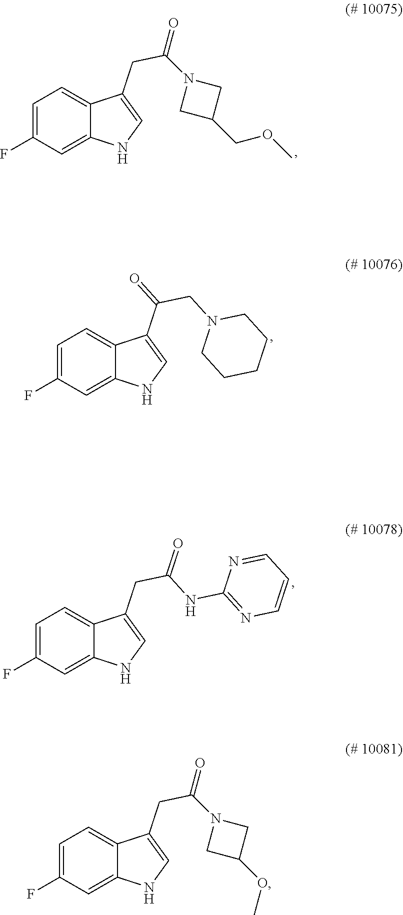 [SMILES: COCC1CN(C(=O)Cc2c[nH]c3cc(F)ccc23)C1 ; O=C(CN1CCCCC1)c1c[nH]c2cc(F)ccc12 ; O=C(Cc1c[nH]c2cc(F)ccc12)Nc1ncccn1 ; COC1CN(C(=O)Cc2c[nH]c3cc(F)ccc23)C1]

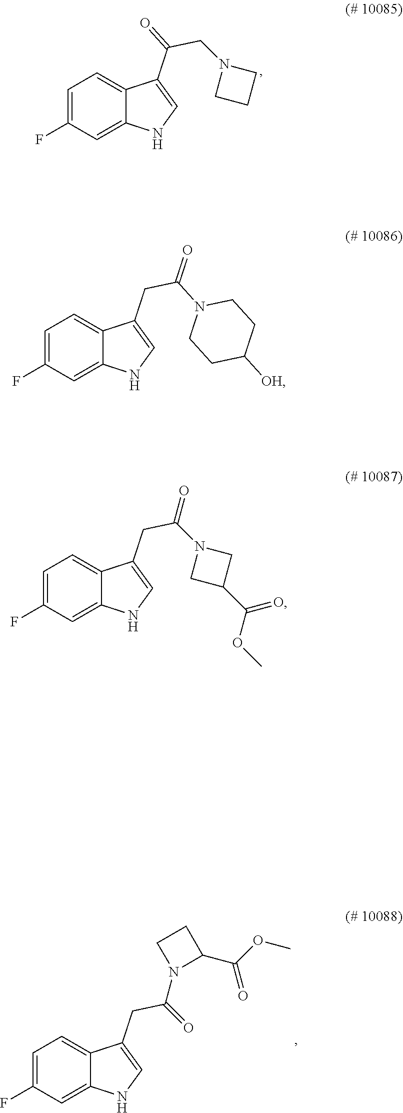 [SMILES: O=C(CN1CCC1)c1c[nH]c2cc(F)ccc12 ; O=C(Cc1c[nH]c2cc(F)ccc12)N1CCC(O)CC1 ; COC(=O)C1CN(C(=O)Cc2c[nH]c3cc(F)ccc23)C1 ; COC(=O)C1CCN1C(=O)Cc1c[nH]c2cc(F)ccc12]

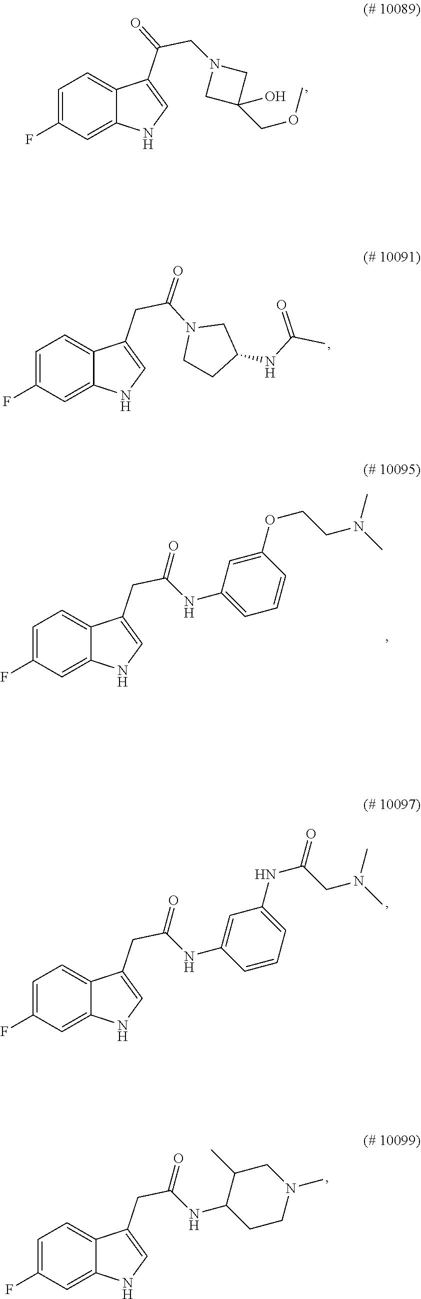 [SMILES: COCC1(O)CN(CC(=O)c2c[nH]c3cc(F)ccc23)C1 ; CC(=O)N[C@@H]1CCN(C(=O)Cc2c[nH]c3cc(F)ccc23)C1 ; CN(C)CCOc1cccc(NC(=O)Cc2c[nH]c3cc(F)ccc23)c1 ; CN(C)CC(=O)Nc1cccc(NC(=O)Cc2c[nH]c3cc(F)ccc23)c1 ; CC1CN(C)CCC1NC(=O)Cc1c[nH]c2cc(F)ccc12]

102.

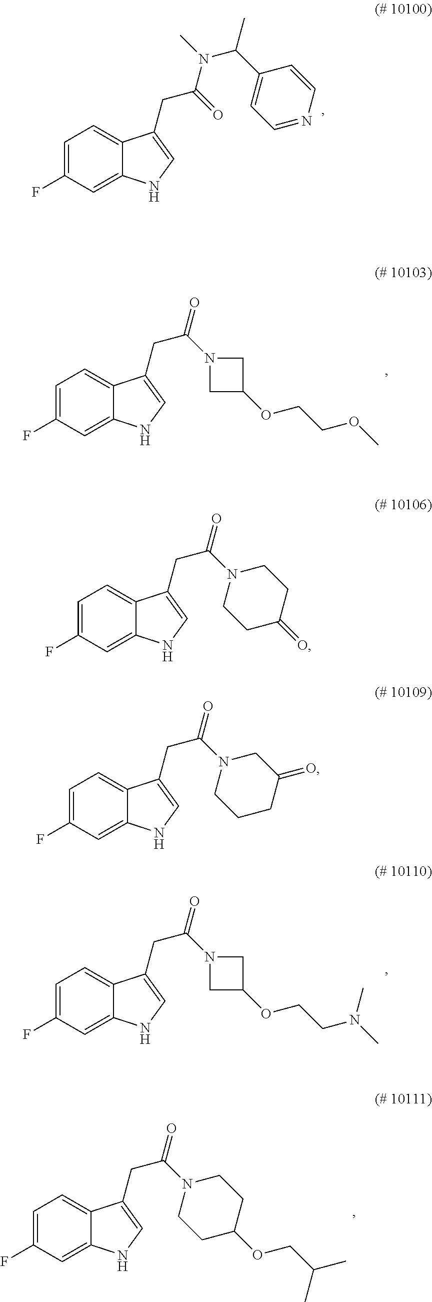 [SMILES: CC(c1ccncc1)N(C)C(=O)Cc1c[nH]c2cc(F)ccc12 ; COCCOC1CN(C(=O)Cc2c[nH]c3cc(F)ccc23)C1 ; O=C1CCN(C(=O)Cc2c[nH]c3cc(F)ccc23)CC1 ; O=C1CCCN(C(=O)Cc2c[nH]c3cc(F)ccc23)C1 ; CN(C)CCOC1CN(C(=O)Cc2c[nH]c3cc(F)ccc23)C1 ; CC(C)COC1CCN(C(=O)Cc2c[nH]c3cc(F)ccc23)CC1]

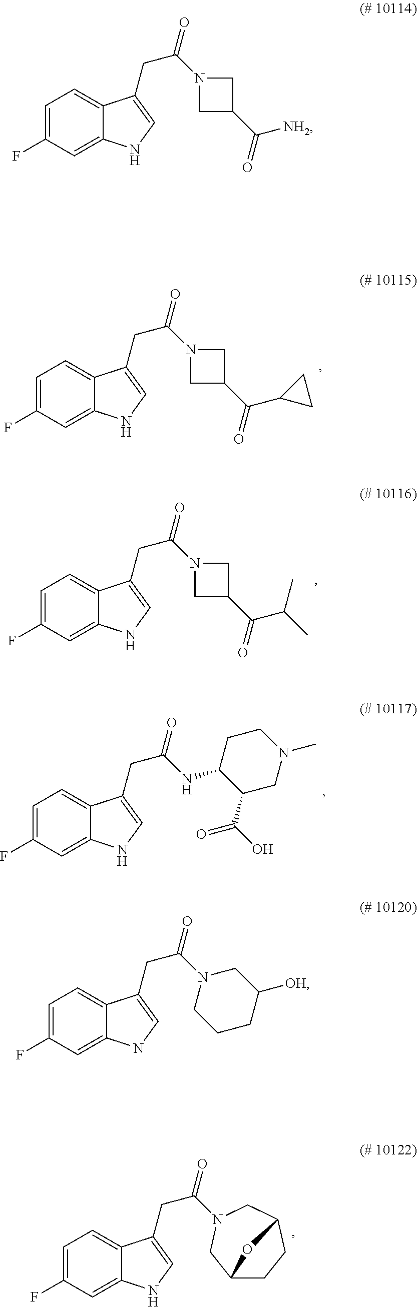 [SMILES: NC(=O)C1CN(C(=O)Cc2c[nH]c3cc(F)ccc23)C1 ; O=C(C1CC1)C1CN(C(=O)Cc2c[nH]c3cc(F)ccc23)C1 ; CC(C)C(=O)C1CN(C(=O)Cc2c[nH]c3cc(F)ccc23)C1 ; CN1CC[C@@H](NC(=O)Cc2c[nH]c3cc(F)ccc23)[C@@H](C(=O)O)C1 ; O=C(Cc1c[nH]c2cc(F)ccc12)N1CCCC(O)C1 ; O=C(Cc1c[nH]c2cc(F)ccc12)N1C[C@H]2CC[C@@H](C1)O2]

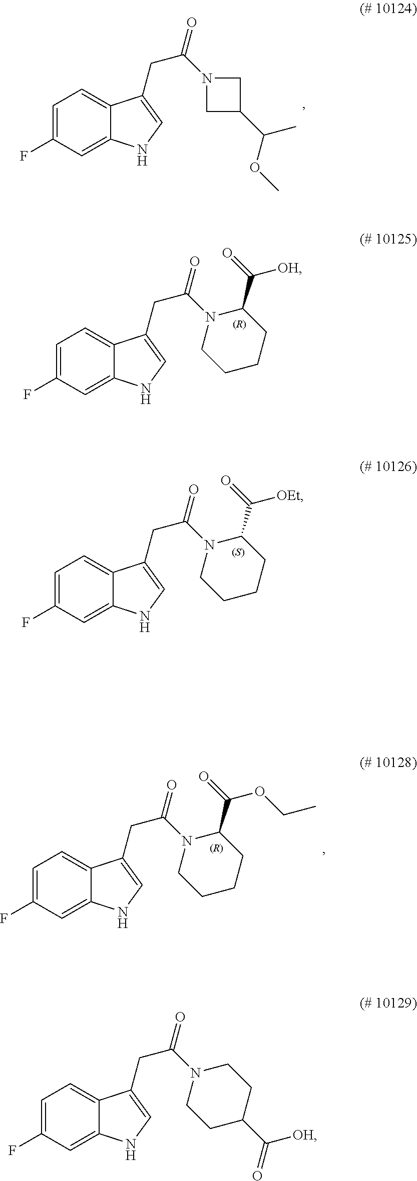

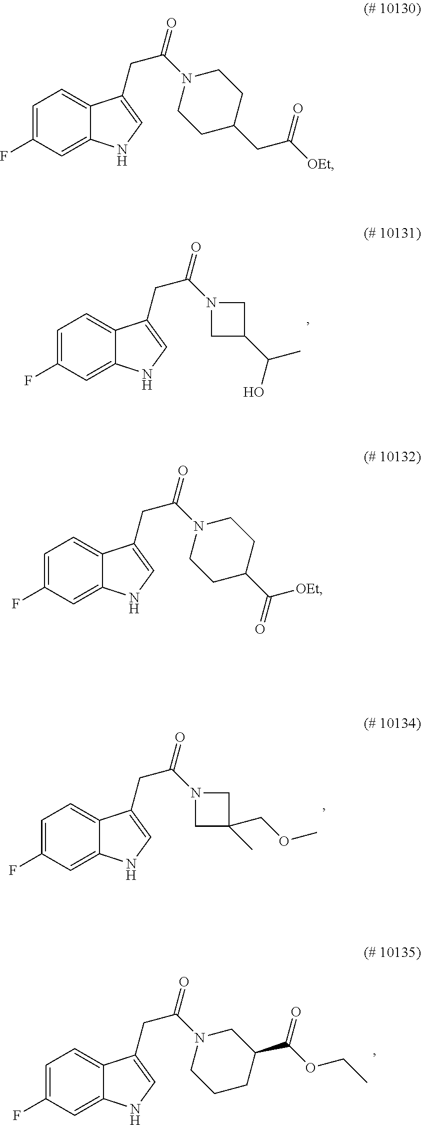 [SMILES: CCOC(=O)CC1CCN(C(=O)Cc2c[nH]c3cc(F)ccc23)CC1 ; CC(O)C1CN(C(=O)Cc2c[nH]c3cc(F)ccc23)C1 ; CCOC(=O)C1CCN(C(=O)Cc2c[nH]c3cc(F)ccc23)CC1 ; COCC1(C)CN(C(=O)Cc2c[nH]c3cc(F)ccc23)C1 ; CCOC(=O)[C@H]1CCCN(C(=O)Cc2c[nH]c3cc(F)ccc23)C1]

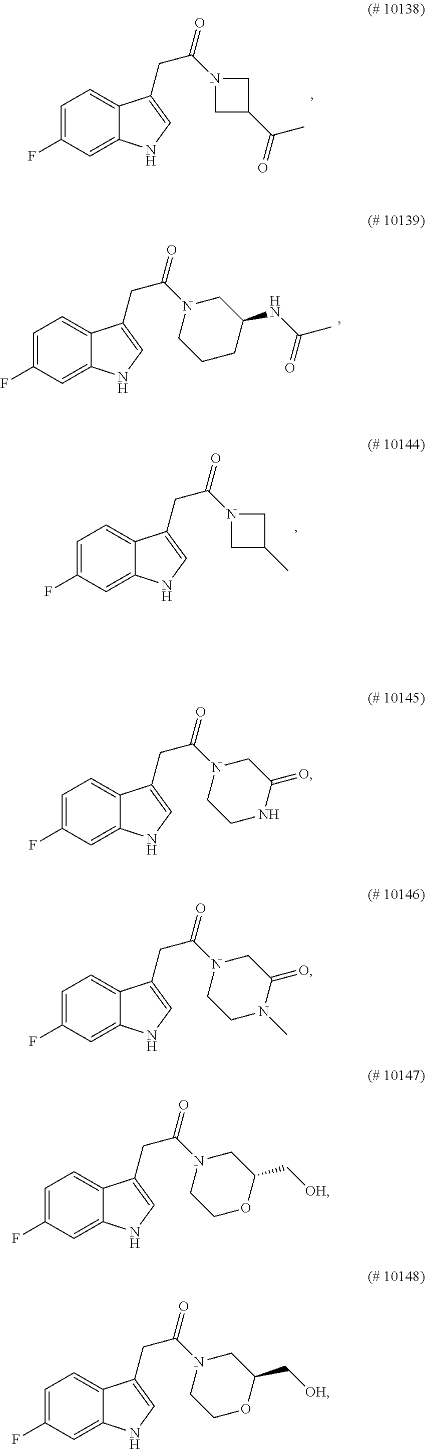

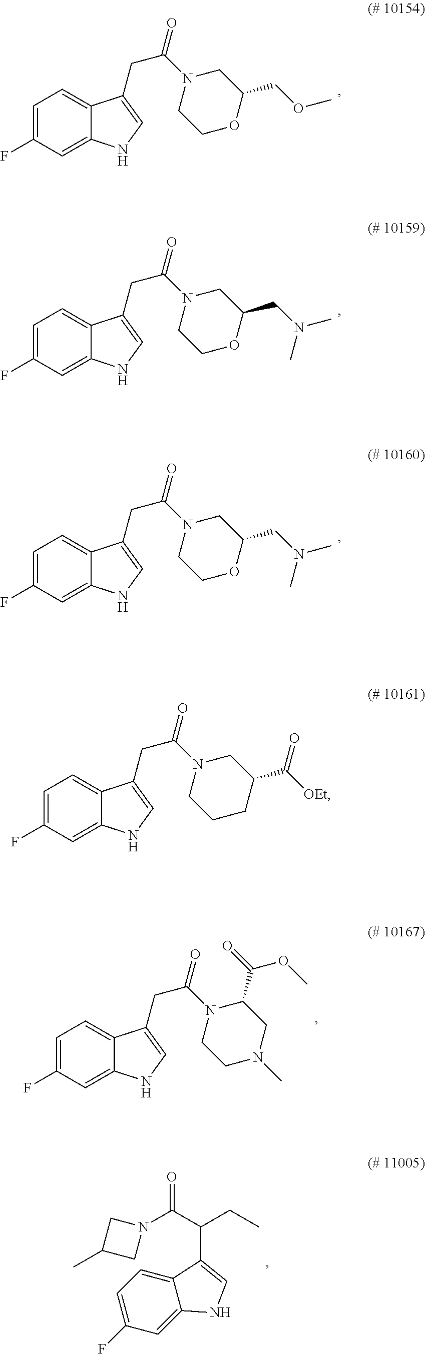

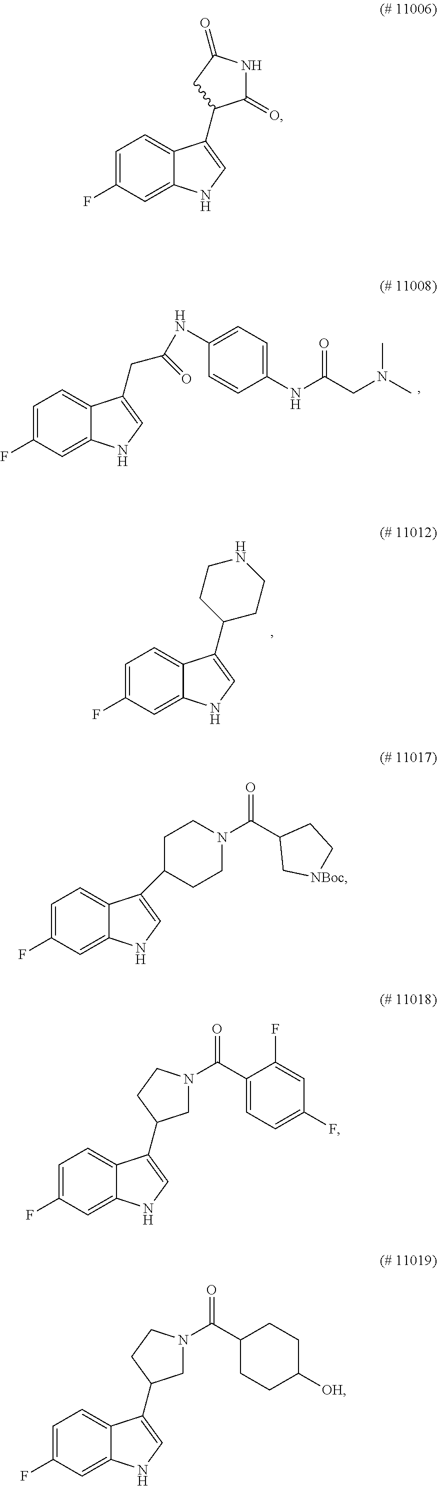 [SMILES: O=C1CC(c2c[nH]c3cc(F)ccc23)C(=O)N1 ; CN(C)CC(=O)Nc1ccc(NC(=O)Cc2c[nH]c3cc(F)ccc23)cc1 ; Fc1ccc2c(C3CCNCC3)c[nH]c2c1 ; CC(C)(C)OC(=O)N1CCC(C(=O)N2CCC(c3c[nH]c4cc(F)ccc34)CC2)C1 ; O=C(c1ccc(F)cc1F)N1CCC(c2c[nH]c3cc(F)ccc23)C1 ; O=C(C1CCC(O)CC1)N1CCC(c2c[nH]c3cc(F)ccc23)C1]

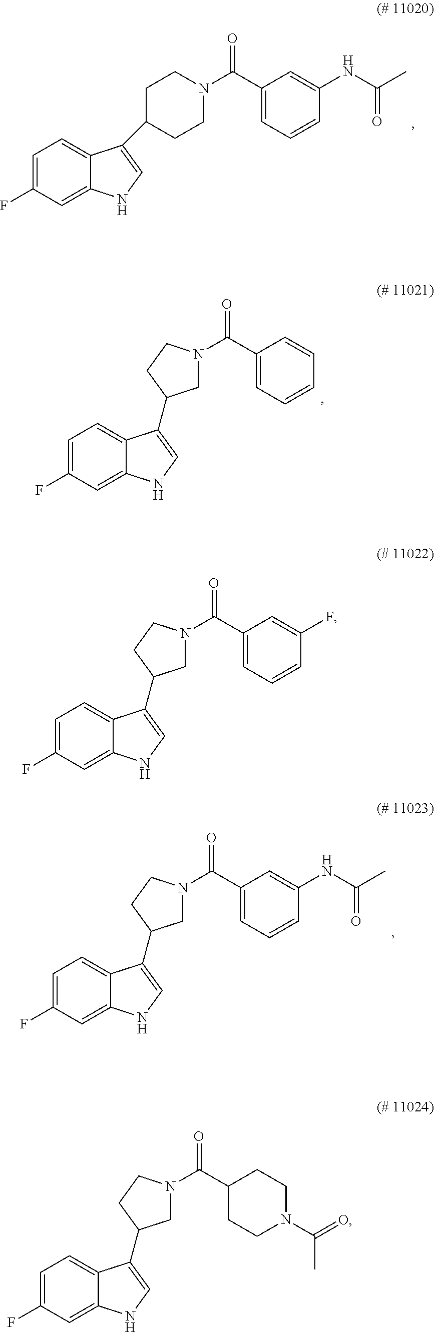 [SMILES: CC(=O)Nc1cccc(C(=O)N2CCC(c3c[nH]c4cc(F)ccc34)CC2)c1 ; O=C(c1ccccc1)N1CCC(c2c[nH]c3cc(F)ccc23)C1 ; O=C(c1cccc(F)c1)N1CCC(c2c[nH]c3cc(F)ccc23)C1 ; CC(=O)Nc1cccc(C(=O)N2CCC(c3c[nH]c4cc(F)ccc34)C2)c1 ; CC(=O)N1CCC(C(=O)N2CCC(c3c[nH]c4cc(F)ccc34)C2)CC1]

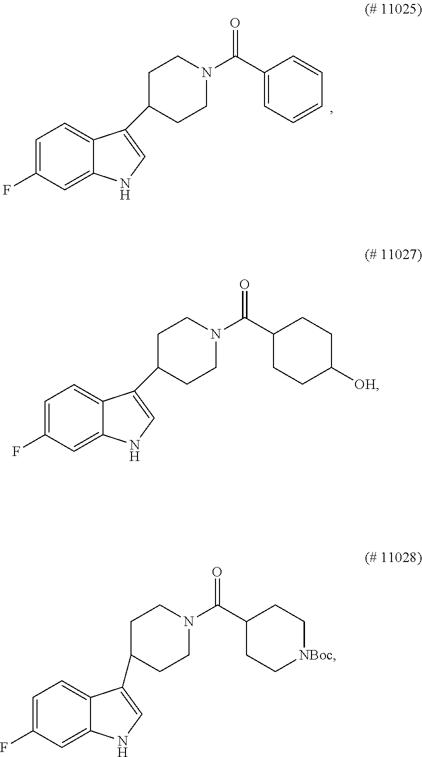 [SMILES: O=C(c1ccccc1)N1CCC(c2c[nH]c3cc(F)ccc23)CC1 ; O=C(C1CCC(O)CC1)N1CCC(c2c[nH]c3cc(F)ccc23)CC1 ; CC(C)(C)OC(=O)N1CCC(C(=O)N2CCC(c3c[nH]c4cc(F)ccc34)CC2)CC1]

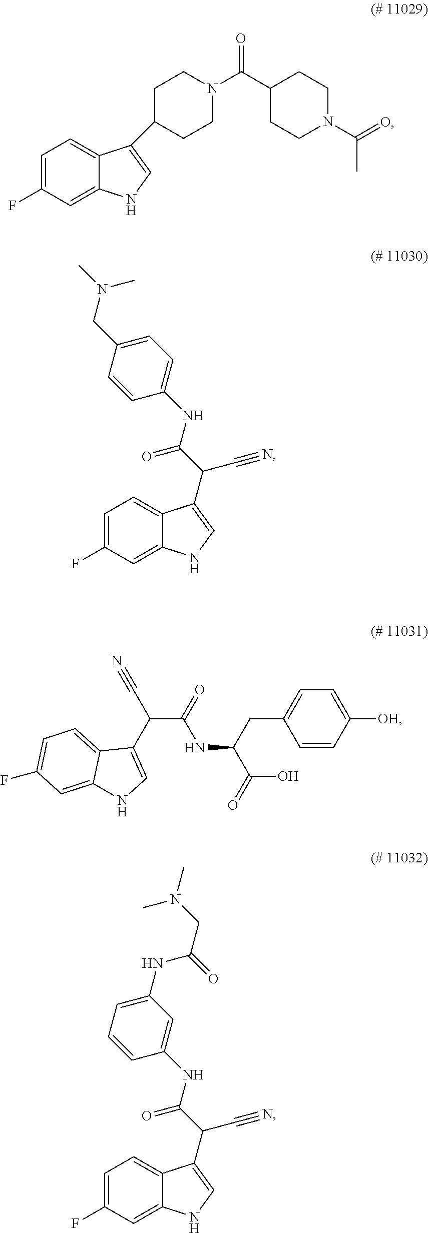 [SMILES: CC(=O)N1CCC(C(=O)N2CCC(c3c[nH]c4cc(F)ccc34)CC2)CC1 ; CN(C)Cc1ccc(NC(=O)C(C#N)c2c[nH]c3cc(F)ccc23)cc1 ; N#CC(C(=O)N[C@@H](Cc1ccc(O)cc1)C(=O)O)c1c[nH]c2cc(F)ccc12 ; CN(C)CC(=O)Nc1cccc(NC(=O)C(C#N)c2c[nH]c3cc(F)ccc23)c1]

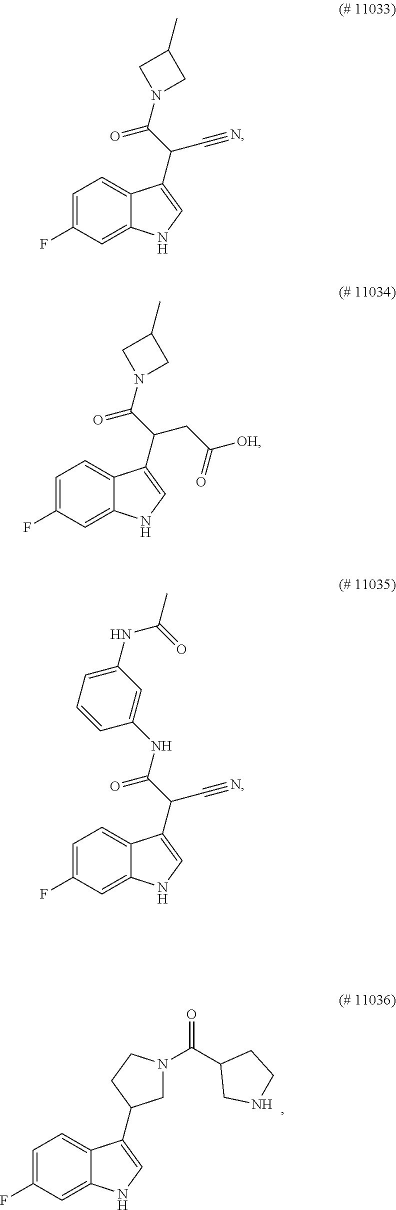 [SMILES: CC1CN(C(=O)C(C#N)c2c[nH]c3cc(F)ccc23)C1 ; CC1CN(C(=O)C(CC(=O)O)c2c[nH]c3cc(F)ccc23)C1 ; CC(=O)Nc1cccc(NC(=O)C(C#N)c2c[nH]c3cc(F)ccc23)c1 ; O=C(C1CCNC1)N1CCC(c2c[nH]c3cc(F)ccc23)C1]

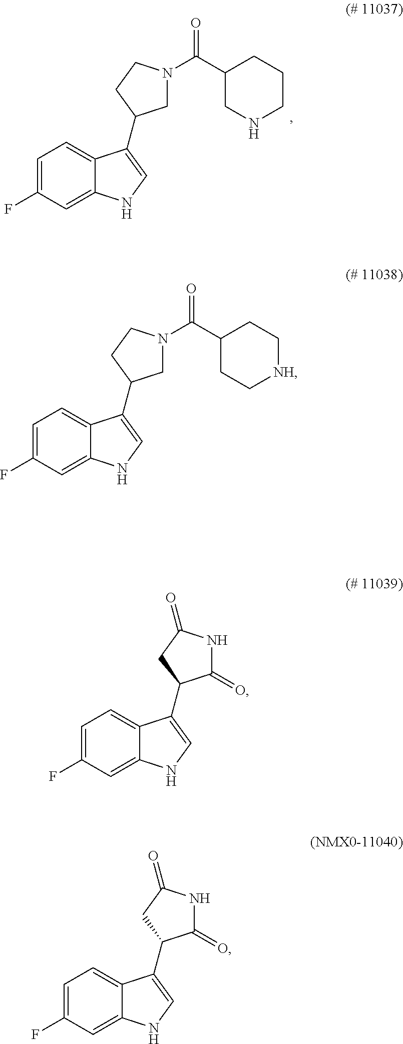 [SMILES: O=C(C1CCCNC1)N1CCC(c2c[nH]c3cc(F)ccc23)C1 ; O=C(C1CCNCC1)N1CCC(c2c[nH]c3cc(F)ccc23)C1 ; O=C1C[C@H](c2c[nH]c3cc(F)ccc23)C(=O)N1 ; O=C1C[C@@H](c2c[nH]c3cc(F)ccc23)C(=O)N1]

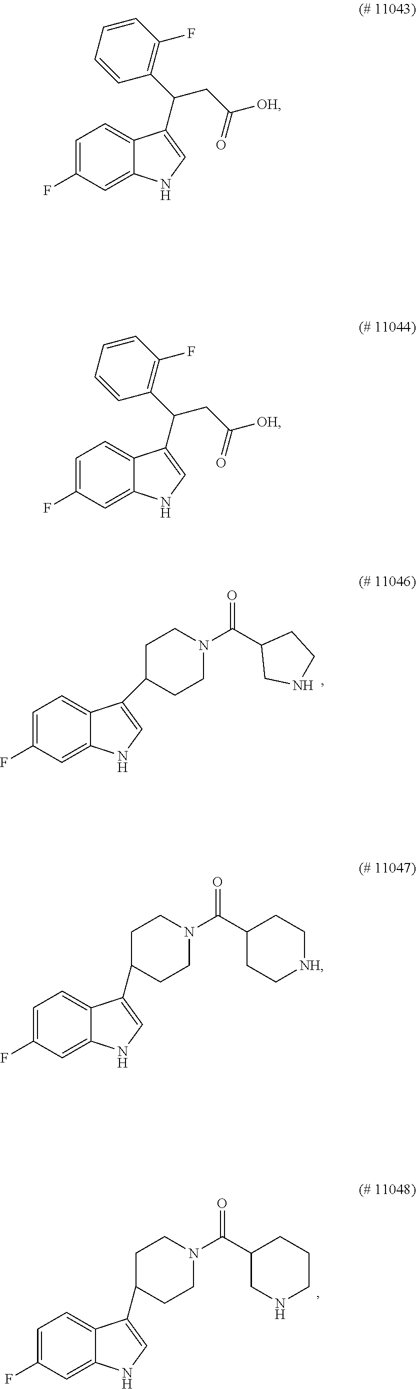 [SMILES: O=C(O)CC(c1ccccc1F)c1c[nH]c2cc(F)ccc12 ; O=C(O)CC(c1ccccc1F)c1c[nH]c2cc(F)ccc12 ; O=C(C1CCNC1)N1CCC(c2c[nH]c3cc(F)ccc23)CC1 ; O=C(C1CCNCC1)N1CCC(c2c[nH]c3cc(F)ccc23)CC1 ; O=C(C1CCCNC1)N1CCC(c2c[nH]c3cc(F)ccc23)CC1]

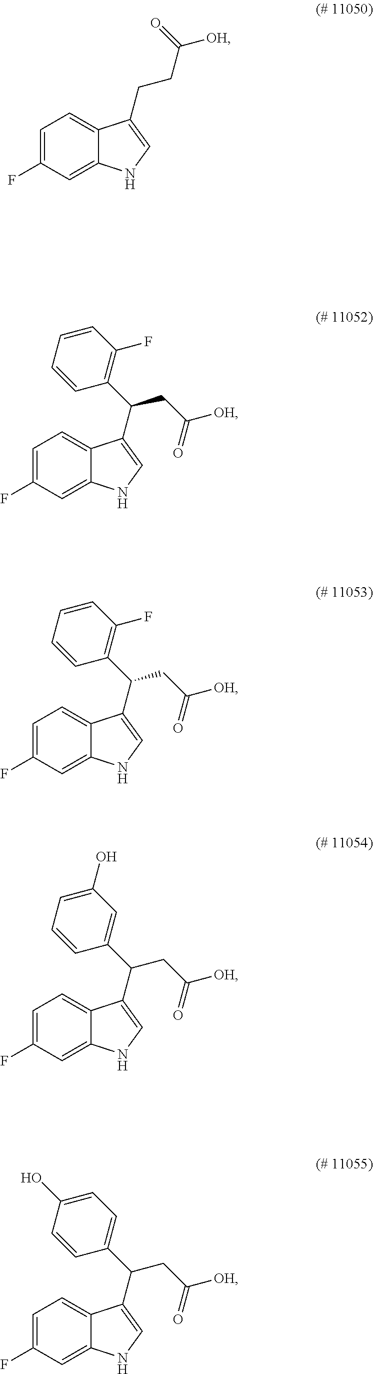 [SMILES: O=C(O)CCc1c[nH]c2cc(F)ccc12 ; O=C(O)C[C@H](c1ccccc1F)c1c[nH]c2cc(F)ccc12 ; O=C(O)C[C@@H](c1ccccc1F)c1c[nH]c2cc(F)ccc12 ; O=C(O)CC(c1cccc(O)c1)c1c[nH]c2cc(F)ccc12 ; O=C(O)CC(c1ccc(O)cc1)c1c[nH]c2cc(F)ccc12]

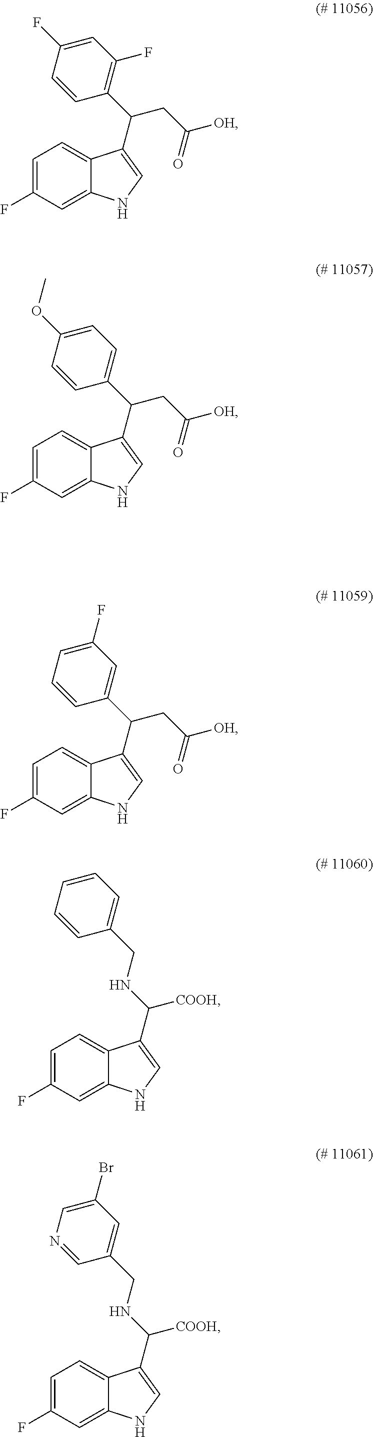 [SMILES: O=C(O)CC(c1ccc(F)cc1F)c1c[nH]c2cc(F)ccc12 ; COc1ccc(C(CC(=O)O)c2c[nH]c3cc(F)ccc23)cc1 ; O=C(O)CC(c1cccc(F)c1)c1c[nH]c2cc(F)ccc12 ; O=C(O)C(NCc1ccccc1)c1c[nH]c2cc(F)ccc12 ; O=C(O)C(NCc1cncc(Br)c1)c1c[nH]c2cc(F)ccc12]

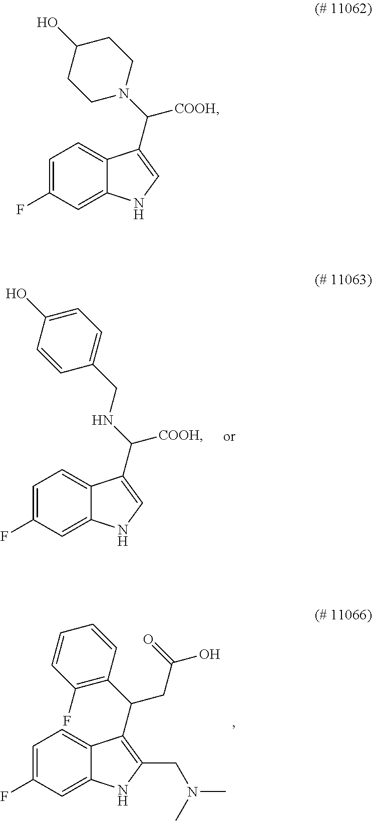 [SMILES: O=C(O)C(c1c[nH]c2cc(F)ccc12)N1CCC(O)CC1 ; O=C(O)C(NCc1ccc(O)cc1)c1c[nH]c2cc(F)ccc12 ; CN(C)Cc1[nH]c2cc(F)ccc2c1C(CC(=O)O)c1ccccc1F]

or a pharmaceutically acceptable salt, ester, solvate, isomer, or tautomer thereof.
103. The compound of embodiment 1 being Compound #10014, 10018, 10035, 10040, 10053, 10066, 10076, 10086, 10095, 10097, 10159, 11005, 11006, 11008, 11043, 11052 (enantiomer of 11043), 11053 (enantiomer of 11043), 11054, 11055, 11066, or 11073, or a pharmaceutically acceptable salt, ester, solvate, isomer, or tautomer thereof.
104. The compound of embodiment 1 being Compound #10018, 10040, 10086, 10095, 10097, 11005, 11006, 11008, 11032, 11053, 11066, or 11073, or a pharmaceutically acceptable salt, ester, solvate, isomer, or tautomer thereof.
105. The compound of embodiment 1 being Compound #10095, 10097, 11005, 11006, 11008, or 11053, or a pharmaceutically acceptable ester, solvate, isomer, or tautomer thereof.
106. The compound of embodiment 1 being Compound #11095, 11097, or 11032, or a pharmaceutically acceptable ester, solvate, isomer, or tautomer thereof.
107. The compound of embodiment 1 being Compound #10018, 10040, 10086, or 11066, or a pharmaceutically acceptable ester, solvate, isomer, or tautomer thereof.
108. A method for inhibiting RAS (wild type or mutant), for example pan-RAS, HRAS, NRAS, and/or KRAS, preferably inhibiting HRAS, and more preferably selectively inhibiting HRAS (or in alternative embodiments selectively inhibiting NRAS, or in yet other alternative embodiments selectively inhibiting KRAS) in a subject in need thereof comprising administering to the subject an effective amount of the compound of any one of embodiments 1 to 107.
109. Use of the compound of any one of embodiments 1 to 107 for inhibiting RAS (wild type or mutant), for example pan-RAS, HRAS, NRAS, and/or KRAS, preferably inhibiting HRAS, and more preferably selectively inhibiting HRAS (or in alternative embodiments selectively inhibiting NRAS, or in yet other alternative embodiments selectively inhibiting KRAS) in a subject, or for the manufacture of a medicament for inhibiting HRAS in a subject.
110. A compound of any one of embodiments 1 to 107 for use in inhibiting RAS (wild type or mutant), for example pan-RAS, HRAS, NRAS, and/or KRAS, preferably inhibiting HRAS, and more preferably selectively inhibiting HRAS (or in alternative embodiments selectively inhibiting NRAS, or in yet other alternative embodiments selectively inhibiting KRAS) in a subject.
111. The method, use and compound of any one of embodiments 108 to 110, being for inhibiting a HRAS mutant, such as HRAS mutated at residue 12.
112. The method, use and compound of embodiment 111, being for inhibiting HRAS G12V mutant.
113. The method, use and compound of any one of embodiments 108 to 112, being for inhibiting NRAS mutant, such as NRAS mutated at residue 61.
114. The method, use and compound of embodiment 113, being for inhibiting NRAS Q61R mutant.
115. The method, use and compound of any one of embodiments 108 to 114, being for inhibiting KRAS mutant, such as KRAS mutated at residue 12.
116. The method, use and compound of embodiment 115, being for inhibiting KRAS G12C or G12D mutant.
117. The method, use and compound of any one of embodiments 108 to 116, being for inhibiting a PAN-RAS mutant, such as PAN-RAS mutated at residue 12.
118. The method, use and compound of embodiment 117, being for inhibiting PAN-RAS G12C or G12D mutant, preferably PAN-RAS G12C mutant.
119. The method, use and compound of any one of embodiments 108 to 110, being for selectively inhibiting HRAS, including wild type HRAS or mutant HRAS.
120. The method, use and compound of embodiment 119, being for selectively inhibiting HRAS mutated at position 12.
121. The method, use and compound of embodiment 120, being for selectively inhibiting HRAS G12V mutant.
122. The method, use and compound of any one of embodiments 108 to 110, being for selectively inhibiting NRAS, including wild type HRAS or mutant NRAS.
123. The method, use and compound of embodiment 122, being for selectively inhibiting NRAS mutated at position 61.
124. The method, use and compound of embodiment 123, being for selectively inhibiting NRAS Q61R mutant.
125. The method, use and compound of any one of embodiments 108 to 110, being for selectively inhibiting KRAS, including wild type HRAS or mutant.
126. The method, use and compound of embodiment 125, being for selectively inhibiting KRAS mutated at position 12.
127. The method, use and compound of embodiment 126, being for selectively inhibiting KRAS G12C or G12D mutant.
128. The method, use and compound of any one of embodiments 108 to 110, being for selectively inhibiting pan-RAS, including wild type HRAS or mutant wild type pan-RAS or mutant pan-RAS.
129. The method, use and compound of embodiment 128, being for selectively inhibiting PAN-RAS mutated at position 12.
130. The method, use and compound of embodiment 129, being for selectively inhibiting PAN-RAS G12C or G12D mutant, preferably PAN-RAS G12C mutant.
131. The method, use and compound of any one of embodiments 108 to 110, being for the prevention or treatment of a disease or disorder associated with abnormal RAS activity, for example abnormal RAS activity caused by a mutation in RAS, preferably Costello syndrome, epidermal nevus (e.g., epidermal nevus sebaceous), giant congenital melanocytic nevus, Noonan syndrome, Noonan syndrome with multiple lentigines, autoimmune lymphoproliferative syndrome, cardiofaciocutaneous syndrome, neurofibromatosis type 1, capillary malformation-arteriovenous malformation syndrome, Legius syndrome or a cancer with a mutated RAS.
132. The method, use and compound of any one of embodiments 108 to 110, being the prevention or treatment of a disease or disorder associated with abnormal HRAS activity, for example abnormal HRAS activity caused by a mutation in HRAS, preferably a mutation at position 12, more preferably Costello syndrome, epidermal nevus (e.g., epidermal nevus sebaceous) or a cancer with a mutated HRAS.
133. The method, use and compound of any one of embodiments 108 to 110, being for the prevention or treatment of a disease or disorder associated with abnormal NRAS activity, for example abnormal NRAS activity caused by a mutation in NRAS, preferably a mutation at position 61, preferably giant congenital melanocytic nevus, Noonan syndrome, autoimmune lymphoproliferative syndrome, epidermal nevus or a cancer with a mutated NRAS.
134. The method, use and compound of any one of embodiments 108 to 110, being for the prevention or treatment of a disease or disorder associated with abnormal KRAS activity, for example abnormal KRAS activity caused by a mutation in KRAS, preferably a mutation at position 12, more preferably cardiofaciocutaneous syndrome, Noonan syndrome, autoimmune lymphoproliferative syndrome, Epidermal nevus, or a cancer with a mutated KRAS.
135. The method, use and compound of any one of embodiments 108 to 110, being for the prevention or treatment of a disease or disorder associated with abnormal PAN-RAS activity, for example abnormal PAN-RAS activity caused by a mutation in PAN-RAS, preferably a mutation at position 12, more preferably the disease or disorder as defined in any one of embodiments 131 to 134.
136. A method for treating cancer in a subject in need thereof comprising administering to the subject an effective amount of the compound of any one of embodiments 1 to 107.
137. Use of the compound of any one of embodiments 1 to 107 for treating cancer in a subject, or
138. Use of the compound of any one of embodiments 1 to 107 for the manufacture of a medicament for treating cancer in a subject.
139. The compound of any one of embodiments 1 to 107 for use in treating cancer in a subject.
140. The method, use and compound of any one of embodiments 136 to 139 being for treating a cancer with a mutated HRAS, preferably bladder, breast, colon, colorectal, cutaneous, embryonal rhabdomyosarcoma, endometrial, glioblastoma, head and neck, leukemia, lung, melanoma (including cutaneous melanoma), oral cavity, ovarian, prostate, renal, salivary duct, skin, or thyroid cancer, more preferably head and neck cancer (preferably head and neck squamous cell carcinoma), thyroid cancer, epithelial-myoepithelial carcinoma, kidney cancer or bladder cancer.
141. The method, use and compound of any one of embodiments 136 to 139 being for treating a cancer with a mutated NRAS, preferably a melanoma (including individuals without giant congenital melanocytic nevus), lung cancer, cholangiocarcinoma, or a hematopoietic malignancy such core binding factor acute myeloid leukemia and cytogenetically normal acute myeloid leukemia, more preferably a melanoma (including individuals without giant congenital melanocytic nevus), lung cancer or a hematopoietic malignancy.
142. The method, use and compound of any one of embodiments 136 to 139 being for treating a cancer with a mutated KRAS, preferably pancreatic cancer, cholangiocarcinoma, Core binding factor acute myeloid leukemia, colorectal cancer, or lung cancer (including non-small cell lung cancer), and more preferably pancreatic cancer, colorectal cancer, or a lung cancer.
143. The method, use and compound of any one of embodiments 136 to 139 being for treating a cancer with a mutated PAN-RAS, preferably a cancer as defined in any one of embodiments 140 to 142.
144. A composition comprising the compound of any one of embodiments 1 to 107 and a pharmaceutically acceptable carrier or excipient.

### BRIEF DESCRIPTION OF THE DRAWINGS

In the appended drawings:

FIG. 1 shows the activity of compound #10095 on healthy bladder cells BdEC.

FIG. 2 shows the activity of compound #11032 on healthy bladder cells BdEC.

FIG. 3 shows the activity of compound #10095 on Bladder Cancer Cells T24 and 5637.

FIG. 4 shows the activity of compound #10097 on Bladder Cancer Cells T24 and 5637

FIG. 5 shows the activity of compound #11032 on Bladder Cancer Cells T24 and 5637

FIG. 6 shows the activity of compound #10095 on healthy bladder cells BdEC and Bladder Cancer Cells T24 and 5637

FIG. 7 A to I shows the morphology of bladder cancer cells (T24) following treatment with decreasing concentrations of compound #10095, A: 200 μM, B: 100 μM, C: 50 μM, D: 25 μM, E: 12.5 μM, F: 6.2 μM, G: 3.1 μM, H: 1.5 μM, and I: control

FIG. 8 A to I shows the morphology of bladder cancer cells (5637) following treatment with decreasing concentrations of compound #10095, A: 200 μM, B: 100 μM, C: 50 μM, D: 25 μM, E: 12.5 μM, F: 6.2 μM, G: 3.1 μM, H: 1.5 μM, and I: control

FIG. 9 A to I shows the morphology of bladder epithelial healthy (BdEC) cells following treatment with decreasing concentrations of compound #10095, A: 200 μM, B: 100 μM, C: 50 μM, D: 25 μM, E: 12.5 μM, F: 6.2 μM, G: 3.1 μM, H: 1.5 μM, and I: control

### DETAILED DESCRIPTION OF THE INVENTION

### Chemical Nomenclature

Herein, the terms “alkyl”, “alkylene”, “alkenyl”, “alkenylene”, “alkynyl”, “alkynylene”, and “cycloalkyl”, “aryl”, “heterocycloalkyl”, “heteroaryl”, and their derivatives (such as alkoxy, alkyleneoxy, etc.) and combinations have their ordinary meaning in the art. For more certainty, herein:

| | |
|---|---|
| Compounds/Radicals | Definition |

Saturated aliphatic hydrocarbons

| | |
|---|---|
| alkane | aliphatic hydrocarbon of general formula CₙH₂ₙ₊₂ |
| alkyl | monovalent alkane radical of general formula —CₙH₂ₙ₊₁ |
| alkylene | bivalent alkane radical of general formula —CₙH₂ₙ₊₁ |

Aliphatic hydrocarbons with double bond(s)

| | |
|---|---|
| alkene | aliphatic hydrocarbon, similar to an alkane but comprising at |
| | least one double bond |
| alkenyl | monovalent alkene radical, similar to an alkyl but comprising at |
| | least one double bond |

Aliphatic hydrocarbons with triple bond(s)

| | |
|---|---|
| alkyne | aliphatic hydrocarbon, similar to an alkane but comprising at |
| | least one triple bond |
| alkynyl | monovalent alkyne radical, similar to an alkyl but comprising at |
| | least one triple bond |

Aliphatic hydrocarbons with double and triple bond(s)

| | |
|---|---|
| alkenyne | aliphatic hydrocarbon, similar to an alkane but comprising at least |
| | one double bond and at least one triple bond |
| alkenynyl | monovalent alkenyne radical, similar to an alkyl but comprising at |
| | least one double bond and at least one triple bond |

Other groups

| | |
|---|---|
| Hydroxy/Hydroxyl | —OH |
| Carbonyl | —C(═O)— |
| Alkoxy | alkyl-O— |
| Carbamoyl | NH₂—C(═O)— |
| Carboxy/carboxyl | OH—C(═O)— |
| Amino | —NH₂ |
| Alkylamino | —NH-alkyl |
| Dialkylamino | —N(alkyl)₂, where the alkyl groups may differ from one another |
| Amido | CH(═O)—NH— |
| Alkanoyl | alkyl-C(═O)— |
| Sulfonyl | —S(═O)₂— |

Cyclic compounds and radicals

| | |
|---|---|
| cycloalkane | monovalent saturated aliphatic hydrocarbon of general formula CₙH₂ₙ, |
| | wherein the carbon atoms are arranged in a ring (also called cycle). |
| Cycloalkanone | aliphatic hydrocarbon, similar to a cycloalkane but wherein at least one |
| | carbon atom is a carbonyl |
| cycloalkene | aliphatic hydrocarbon, similar to a cycloalkane but comprising at least one |
| | double bond |
| cycloalkyne | aliphatic hydrocarbon, similar to a cycloalkane but comprising at least one |
| | triple bond |
| cycloalkenyne | aliphatic hydrocarbon, similar to a cycloalkane but comprising at least one |
| | double bond and one triple bond |
| cycloalkyl, | monovalent cycloalkane, cycloalkene, cycloalkyne, and cycloalkenyne |
| cycloalkenyl, | radicals, respectively |
| cycloalkynyl. | |
| cycloalkenynyl | |
| heterocycloalkane, | cycloalkane, cycloalkene, cycloalkyne, and cycloalkenyne, respectively |
| heterocycloalkene, | wherein at least one of the carbon atoms is replaced by a heteroatom. |
| heterocycloalkyne, | |
| heterocycloalkenyne | |
| heterocycloalkyl, | monovalent heterocycloalkane, heterocycloalkene, heterocycloalkyne, and |
| heterocycloalkenyl, | heterocycloalkenyne radicals, respectively |
| heterocycloalkynyl, | |
| heterocycloalkenynyl | |
| heterocycloalkanonyl, | aliphatic hydrocarbon, similar to a heterocycloalkane, heterocycloalkene, |
| heterocycloalkenonyl, | heterocycloalkyne, and heterocycloalkenyne respectively, but wherein at |
| heterocycloalkynonyl, | least one carbon atom is a carbonyl |
| heterocycloalkenynonyl | |
| arene | aromatic hydrocarbon presenting alternating double and single bonds |
| | between carbon atoms arranged in one or more rings. Arenes present pi |
| | bonds in resonance that give increased stability compared to other |
| | geometric or connective arrangements with the same set of atoms. |
| aryl | monovalent arene radical |
| heteroarene | arene wherein at least one of the carbon atoms forming the ring(s) is |
| | replaced by a heteroatom. In heteroarenes, one or more of the double |
| | bonds can be replaced by a corresponding of heteroatoms as long as the |
| | pi bonds remain in resonance. |
| heteroaryl | monovalent heteroarene radical |

Examples of combinations

| | |
|---|---|
| Hydroxyalkyl | HO-alkyl- |
| Alkoxyalkyl | alkyl-O-alkyl- |
| Alkoxyalkoxy | alkyl-O-alkyl-O— |
| Alkylcarbonyl | alkyl-C(═O)— |
| Alkoxycarbonyl | alkyl-O—C(═O)— |
| Alkoxycarbonylalkyl | alkyl-O—C(═O)-alkyl- |
| Carboxyalkyl | OH—C(═O)-alkyl- |
| Carbamoylalkyl | NH₂—C(═O)-alkyl- |
| N-alkylcarbamoyl | alkyl-NH—C(═O)— |
| Aminoalkyl, | NH₂-alkyl- |
| N-alkylaminoalkyl, | NH(alkyl)-alkyl- |
| N-dialkylaminoalkyl | N(alkyl)₂-alkyl- |
| Aminoalkylcarbamoyl, | —NH—C(═O)-alkyl-NH₂ |
| N-alkylaminoalkylcarbamoyl, | —NH—C(═O)-alkyl-NH(alkyl) |
| N-dialkylaminoalkylcarbamoyl | —NH—C(═O)-alkyl-N(alkyl)₂ |
| Aminoalkoxy, | NH₂-alkyl-O— |
| N-alkylaminoalkoxy, | NH(alkyl)-alkyl-O— |
| N-dialkylaminoalkoxy | N(alkyl)₂-alkyl-O— |
| Alkylamido | alkyl-C(═O)—NH— |
| Alkoxyalkylamido | alkoxy-alkyl-C(═O)—NH— |
| Alkylamidoalkyl | alkyl-C(═O)—NH-alkyl- |
| Alkoxyalkylamino | alkyl-O-alkyl-NH— |
| Arylalkyl | aryl-alkyl- |
| Heteroarylalkyl | heteroaryl-alkyl |
| heterocycloalkylalkyl | heterocycloalkyl-alkyl- |
| Heterocycloalkylcarbonyl | heterocycloalkyl-C(═O)— |
| Heterocycloalkylcarbonylalkyl | heterocycloalkyl-C(═O)-alkyl- |
| Alkylsulfonyl | —S(=O)₂-alkyl |
| Alkylsulfonylamino | —NH—S(═O)₂-alkyl |

Herein, a “heteroatom” is an atom other than a carbon atom or a hydrogen atom. Preferably, the heteroatom is oxygen or nitrogen.

Herein, a “ring atom”, such as a ring carbon atom or a ring heteroatom, refers to an atom that forms (with other ring atoms) a ring of a cyclic compound, such as a cycloalkyl, an aryl, etc.

It is to be noted that, unless otherwise specified, the hydrocarbon chains of the above groups can be linear or branched. Further, unless otherwise specified, these groups can contain between 1 and 18 carbon atoms, more specifically between 1 and 12 carbon atoms, between 1 and 6 carbon atoms, between 1 and 3 carbon atoms, or contain 1 or 2, preferably 1, or preferably 2 carbon atoms.

It is also to be noted that, unless otherwise specified, each ring in the above cyclic compounds can comprise between 4 and 8 ring atoms, preferably 4, 5 or 6 ring atoms. Also, each of the above cyclic compounds may comprise more than one ring. These cyclic compounds can be spirocyclic compounds, in which two rings share only a single atom, the spiro atom, which is usually a quaternary carbon. They can also be fused compounds, in which two rings are fused together by sharing two neighboring carbon atoms. They can also be bridged compounds, in which two rings share three or more atoms, separating two bridgehead atoms by a bridge containing at least one atom.

Non-limiting examples of heterocycloalkyls and heterocycloalkenyls include the following as well as the same compounds but with the radical (i.e. the point of attachment to the rest of the compound of the invention) located on any other ring atoms:

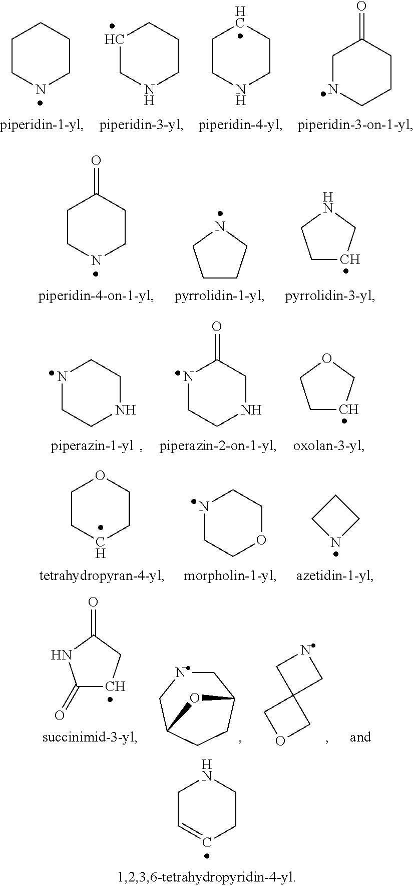

Non-limiting examples of heterocycloalkanonyls include the following as well as the same compounds but with the radical (i.e., the point of attachment to the rest of the compound of the invention) located on any other ring atoms:

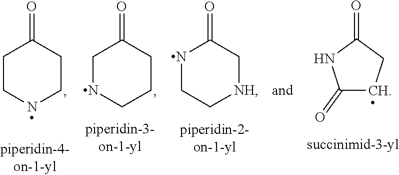

Non-limiting examples of heteroaryls include the following as well as the same compounds but with the radical (i.e., the point of attachment to the rest of the compound of the invention) located on any other ring atoms:

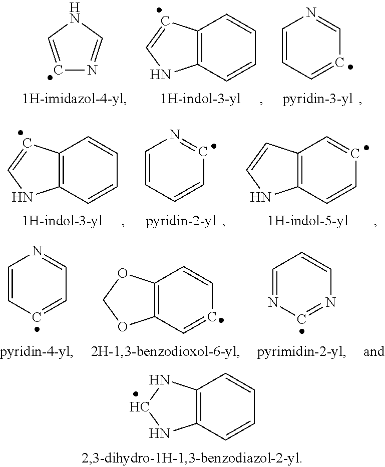

### Compounds of the Invention

Turning now to the invention in more details, there is provided a compound of formula (I):

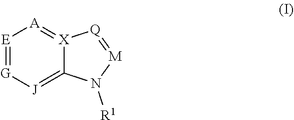

wherein:
R¹ represents H, alkoxycarbonyl, alkylcarbonyl, Cₙ-alkyl, wherein n is 2 or more, or carbamoyl,
A represents —C(R²)═,
E represents —N═ or —C(R³)═,
G represents —N═ or —C(R⁴)═,
J represents —C(R⁵)═,
X represents —C(R⁶)═,
M represents —N═ or —C(R⁷)═,
Q represents —N═ or —C(R⁸)═,
with the proviso that no more than four (4) of A, E, G, J, X, M, and Q represent —N═,
each of R⁴, R⁵, R⁶, and R⁸ independently represents H, a halogen atom, alkyl, alkenyl, alkynyl, alkenynyl, hydroxyl, —OR⁹, or -L-R¹⁰,
R² represents H, a halogen atom, alkyl, alkenyl, alkynyl, alkenynyl, alkyl-N(R⁹)₂, alkenyl-N(R⁹)₂, alkynyl-N(R⁹)₂, alkenynyl-N(R⁹)₂, hydroxyl, or —OR⁹,
R³ represents H, alkyl, alkenyl, alkynyl, alkenynyl, alkyl-N(R⁹)₂, alkenyl-N(R⁹)₂, alkynyl-N(R⁹)₂, alkenynyl-N(R⁹)₂, hydroxyl, or —OR⁹,
R⁷ represents H, a halogen atom, Cₙ-alkyl, wherein n is 2 or more, alkenyl, alkynyl, alkenynyl, alkyl-N(R⁹)₂, alkenyl-N(R⁹)₂, alkynyl-N(R⁹)₂, alkenynyl-N(R⁹)₂, hydroxyl, or —OR⁹,
R⁹ represents H, alkyl, alkenyl, alkynyl, or alkenynyl, each of the alkyl, alkenyl, alkynyl, and alkenynyl being optionally substituted with R¹⁶,
with the proviso that the compound of formula (I) comprises exactly one or exactly two -L-R¹⁰ group(s) identical or different from one another,
each L independently represents a covalent bond or a chain comprising any combination of the following:
   up to two —N(R¹¹)— group,
   up to one —C(═O)—, —O—C(═O)—, —C(═O)—O—, —SO₂—, or —SO— group, and
   up to five —C(R¹²)₂— groups, wherein the —C(R¹²)₂— groups can be adjacent to one another or separated by —N(R¹¹)—, —C(═O)—, and/or —S(═O)₂ groups,
wherein, in each -L-R¹⁰ group independently:
A) R¹⁰ represents:
   a 6-membered cycle selected from cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl, each of which comprising one or more nitrogen ring atoms as sole heteroatoms, and each of which being independently unsubstituted or substituted with
      a halogen atom, —CN, hydroxyl, —N(R¹⁵)₂, —N(R¹⁵)—C(═O)—R¹⁸, —C(═O)—N(R¹⁵)₂, —C(═O)—R¹⁷, —C(═O)—OR¹⁷, —OR¹⁵, —O—C(═O)—R¹⁷, —SO₂—R¹⁵, —SO—R¹⁵, —N(R¹⁵)₂—SO₂—R¹⁵, or —N(R¹⁵)₂—SO—R¹⁵, or
      alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, or heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R¹⁶,
   or
   aryl or heteroaryl, each of which independently unsubstituted or substituted with, in a position other than in the position immediately next to the ring atom attached to L:
      alkyl-N(R¹⁵)₂, alkenyl-N(R¹⁵)₂, alkynyl-N(R¹⁵)₂, alkenynyl-N(R¹⁵)₂, —N(R¹⁵)₂, —N(R¹⁵)—C(═O)—R¹⁵, —C(═O)—N(R¹⁵)₂, —C(═O)—R¹⁵, —C(═O)—OR¹⁷, —OR¹⁸, —O—C(═O)—R¹⁵, —SO₂—R¹⁵, —SO—R¹⁵, —N(R¹⁵)₂—SO₂—R¹⁵, —N(R¹⁵)₂—SO—R¹⁵, or
      cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of independently unsubstituted or substituted with one or more R¹⁶,
   with the proviso that the aryl or heteroaryl are substituted with no more than one —OR¹⁸ group, and
   with the proviso that -L-R¹⁰ is not

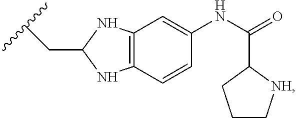 [SMILES: *CC1Nc2ccc(NC(=O)C3CCCN3)cc2N1]

L represents a chain of at most 5 atoms in length,
each R¹¹ independently represents H, or
   alkyl, alkenyl, alkynyl, or alkenynyl, each of which independently unsubstituted of substituted with one or more R³⁰,
each R¹² independently represents H, —C≡N, —N(R¹⁵)₂, -T-COOR¹⁴, or
   alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰,
B) R¹⁰ represents —COOH,
   L represents a chain that is at most 3 atoms in length, and that is not —CH₂—CH(NH₂)—,
   any R¹¹ independently represents H, or
      alkyl, alkenyl, alkynyl, or alkenynyl, each of which independently unsubstituted or substituted with one or more R³⁰, and
   any R¹² independently represents H, —C≡N, —N(R¹⁵)₂, —C(═O)—R¹⁵, or
      alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰,
C) R¹⁰ is attached to a nitrogen atom of a —N(R¹¹)— group that ends the chain in L, and R¹⁰ and the R¹¹ of the —N(R¹¹)— group that ends the chain in L together with the nitrogen atom to which they are attached form a heteroaryl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl, each of which independently unsubstituted or substituted with:
   a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR²⁰, —OR¹⁵, —O—C(═O)—R¹⁵, —SO₂—R¹⁵, —SO—R¹⁵, —N(R¹⁵)₂—SO₂—R¹⁵, —N(R¹⁵)₂—SO—R¹⁵, or
   alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R²¹,
   with the proviso that when R¹⁰ and R¹¹ together with the nitrogen atom to which they are attached form morpholinyl, the morpholinyl is free of a substituent containing a —C(═O)— group,
   with the proviso that when R¹⁰ and R¹¹ together with the nitrogen atom to which they are attached form piperazinyl substituted with a —C(═O)—O—CH₃ group, the —C(═O)—O—CH₃ group is in position 2,
   with the proviso that when R¹⁰ and R¹¹ together with the nitrogen atom to which they are attached form piperazinyl N-substituted with —O-heterocycloalkyl, the heterocycloalkyl is other than oxalanyl,
the chain in L is at most 4 atoms in length and ends with the —N(R¹¹)— group to which R¹⁰ is attached,
   with the proviso that -L-R¹⁰ is not

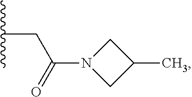 [SMILES: *CC(=O)N1CC(C)C1]

any other R¹¹ independently represents H, or
   alkyl, alkenyl, alkynyl, or alkenynyl, each of which independently unsubstituted or substituted with one or more R³¹ and
any R¹² independently represents H, —C≡N, —N(R¹⁵)₂, or
   alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, eterocycloalkenynonyl, aryl, or heteroaryl, each of which independently unsubstituted or substituted with one or more R³¹,
   with the proviso that when one R¹² on a given carbon atom is methyl, any other R¹² on said given carbon atom is other than methyl,
or
D) R¹⁰ represents H, a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR¹⁵, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵, or
   alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each independently unsubstituted or substituted with one or more R¹⁶,
   the chain in L is at most 3 atoms in length, contains at least one —C(R¹²)₂— group, and ends with a —N(R¹¹)— group,
   the R¹¹ of the —N(R¹¹)— group that ends the chain in L and one R¹² of said at least one —C(R¹²)₂— group together with the one or more atoms to which they are attached and any atom(s) between said one or more atoms form heterocycloalkyl, heterocycloalkanonyl, heterocycloalkenonyl, heterocycloalkynonyl, heterocycloalkenynonyl, or heteroaryl, each of which being independently unsubstituted or substituted with:
      a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR¹⁵, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵,
      alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which being independently unsubstituted or substituted with one or more R¹⁶,
      with the proviso that when said one R¹¹ and said one R¹² form pyrrolidinyl, the pyrrolidinyl is substituted,
      with the proviso that when the heterocycloalkyl is piperidinyl, R¹⁰ is other than 2,4-difluorophenylcarbonyl substituent,
   any other R¹¹ independently represent H or
      alkyl, alkenyl, alkynyl, alkenynyl, each of which independently unsubstituted or substituted with one or more R³¹, and
   any other R¹² independently represent H, —C≡N, or —N(R¹⁵)₂,
      alkyl, alkenyl, alkynyl, or alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³¹
   with the proviso that -L-R¹⁰ is not

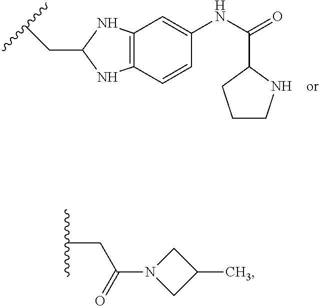

wherein R¹⁴ represents H, alkyl, alkenyl, alkynyl, or alkenynyl,
wherein T represents a covalent bond, alkylene, alkenylene, alkynylene, or alkenynylene,
wherein each R³⁰ independently represents:
   a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR¹⁵, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵,
   alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R¹⁶,
wherein each R³¹ independently represents:
   a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR²⁰, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵,
   alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R¹⁶,
wherein each R¹⁵ independently represents H, or
   alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R¹⁶
wherein each R¹⁷ independently represents cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, aryl, or heteroaryl, each of which independently unsubstituted or substituted with one or more R¹⁶,
wherein each R²⁰ independently represents alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, aryl, or heteroaryl, or, each of which independently unsubstituted or substituted with one or more R¹⁶ wherein each R¹⁸ independently represents H, or
alkyl, alkenyl, alkynyl, alkenynyl, cycloalkyl, cycloalkenyl, cycloalkynyl, cycloalkenynyl,
   cycloalkanonyl, cycloalkenonyl, cycloalkynonyl, cycloalkenynonyl, heterocycloalkyl, heterocycloalkenyl, heterocycloalkynyl, heterocycloalkenynyl, aryl, or heteroaryl, each of which independently unsubstituted or substituted with one or more R¹⁹,
wherein each R¹⁶ independently represents a halogen atom, alkoxy, alkylcarbonyl, alkenylcarbonyl, alkynylcarbonyl, alkenynylcarbonyl, alkoxycarbonyl, alkyl, alkenyl, alkynyl, alkenynyl, carbamoyl, alkylcarbamoyl, alkenylcarbamoyl, alkynylcarbamoyl, alkenynylcarbamoyl, aryl (optionally substituted with one or more halogen atoms), heteroaryl (optionally substituted with one or more halogen atoms), —CN, carboxyl, hydroxyl, amido, alkylamido, dialkylamido, alkenylamido, dialkenylamido, alkynylamido, dialkynylamido, alkenynylamido, dialkenynylamido, amino, alkylamino, dialkylamino, alkenylamino, dialkenylamino, alkynylamino, dialkynylamino, alkenynylamino, dialkenynylamino, alkylsulfonylamino, alkenylsulfonylamino, alkynylsulfonylamino, or alkenynylsulfonylamino,
wherein each R¹⁹ independently represents a halogen atom, alkylcarbonyl, alkenylcarbonyl, alkynylcarbonyl, alkenynylcarbonyl, alkoxycarbonyl, alkyl, alkenyl, alkynyl, alkenynyl, carbamoyl, alkylcarbamoyl, alkenylcarbamoyl, alkynylcarbamoyl, alkenynylcarbamoyl, aryl (optionally substituted with one or more halogen atoms), heteroaryl (optionally substituted with one or more halogen atoms), —CN, carboxyl, hydroxyl, amido, alkylamido, dialkylamido, alkenylamido, dialkenylamido, alkynylamido, dialkynylamido, alkenynylamido, dialkenynylamido, amino, alkylamino, dialkylamino, alkenylamino, dialkenylamino, alkynylamino, dialkynylamino, alkenynylamino, dialkenynylamino, alkylsulfonylamino, alkenylsulfonylamino, alkynylsulfonylamino, or alkenynylsulfonylamino
wherein each R²¹ independently represents a halogen atom, alkoxy, alkylcarbonyl, alkenylcarbonyl, alkynylcarbonyl, alkenynylcarbonyl, alkoxycarbonyl, alkyl, alkenyl, alkynyl, alkenynyl, carbamoyl, alkylcarbamoyl, alkenylcarbamoyl, alkynylcarbamoyl, alkenynylcarbamoyl, aryl (optionally substituted with one or more halogen atoms), heteroaryl (optionally substituted with one or more halogen atoms), —CN, hydroxyl, amido, alkylamido, dialkylamido, alkenylamido, dialkenylamido, alkynylamido, dialkynylamido, alkenynylamido, dialkenynylamido, amino, alkylamino, dialkylamino, alkenylamino, dialkenylamino, alkynylamino, dialkynylamino, alkenynylamino, dialkenynylamino, alkylsulfonylamino, alkenylsulfonylamino, alkynylsulfonylamino, or alkenynylsulfonylamino,
   or a pharmaceutically acceptable salt, ester, solvate, isomer, or tautomer thereof.

In preferred embodiments, the compound of formula (I) comprises exactly one -L-R¹⁰ group.

In alternative embodiments, the compound of formula (I) comprises exactly two -L-R¹⁰ group.

As noted above, R¹ represents H, alkoxycarbonyl, alkylcarbonyl, Cₙalkyl, wherein n is 2 or more, or carbamoyl. A preferred alkoxycarbonyl is tert-butyloxycarbonyl (t-BOC). A preferred alkyl is methyl. In preferred embodiments, R¹ represents H.

In embodiments, A represents —CH═.

In embodiments, E represents —C(R³)═, preferably —C(H)═.

In embodiments, G represents —C(R⁴)═, preferably wherein R⁴ represents a halogen atom, more preferably F.

In embodiments, J represents —CH═.

In embodiments, X represents —C(H)═.

In preferred embodiments, M represents —C(R⁷)═, wherein R^{y7} preferably represents H. In alternative embodiments, M represents —N═.

In embodiments, Q represents —C(R⁸)═, preferably —C(-L-R¹⁰)═.

In embodiments, the compound is a compound of category A) wherein R¹⁰ is a 6-membered cycle or aryl/heteroaryl, category C) wherein R¹⁰ and R¹¹ form a heterocycle, or category D wherein R¹¹ and R¹² form a heterocycle. In preferred embodiments, the compound is a compound of category A) wherein R¹⁰ is a 6-membered cycle.

Compounds with A) R¹⁰=6-membered cycle or aryl/heteroaryl

As noted above, in embodiments A), R¹⁰ represents a 6-membered cycle or aryl/heteroaryl.

In embodiments, M represents —C(R⁷)═, preferably wherein R⁷ represents H or alkyl-N(R⁹)₂. In more preferred embodiments, both R⁹ in alkyl-N(R⁹)₂ represent alkyl, preferably methyl. In most preferred embodiments, M thus represents —C(—CH₂—N(CH₃)₂)═. In alternative preferred embodiments, R⁷ represents H or alkyl (preferably methyl), preferably R⁷ represents H.

In embodiments, the chain in L is at most 4 atoms in length, preferably at most 3 atoms in length.

In embodiments, the chain in L is at least 1 atom in length, preferably at least 2 atoms in length.

In preferred embodiments, the chain in L is 3 atoms in length

In embodiments, L represents:
—C(R¹²)₂—N(R¹¹)—,
—C(R¹²)₂—C(═O)—N(R¹¹)—,
—C(R¹²)₂—N(R¹¹)—C(═O)—,
—C(R¹²)₂—C(R¹²)₂—N(R¹¹)—C(═O)—,
—C(R¹²)₂—C(R¹²)₂—C(═O)—N(R¹¹)—,
—C(R¹²)₂—N(R¹¹)—C(═O)—C(R¹²)₂—,
—C(R¹²)₂—C(═O)—N(R¹¹)—C(R¹²)₂—,
—C(R¹²)₂—C(═O)—N(R¹¹)—C(R¹²)₂—C(R¹²)₂—, or
—C(R¹²)₂—N(R¹¹)—C(═O)—C(R¹²)₂—C(R¹²)₂—,
   preferably:
—C(R¹²)₂—N(R¹¹)—,
—C(R¹²)₂—C(═O)—N(R¹¹)—,
—C(R¹²)₂—C(R¹²)₂—N(R¹¹)—C(═O)—,
—C(R¹²)₂—C(═O)—N(R¹¹)—C(R¹²)₂— or
—C(R¹²)₂—C(═O)—N(R¹¹)—C(R¹²)₂—C(R¹²)₂—,
   and most preferably —C(R¹²)₂—C(═O)—N(R¹¹)—.

In embodiments, L is free of a —N(R¹¹)— group in which R¹¹ forms a cycle with R¹⁰ or R¹².

In embodiments, the compound is free of a R¹² that represents -T-COOH, preferably -T-COOR¹⁴.

In preferred embodiments, all R¹² represent H.

In alternative embodiments, one R¹² on a carbon atom is H and the other R¹² on the carbon atom is —C≡N, alkyl or, for aryls/heteroaryls only, -T-COOR¹⁴. In preferred such embodiments, one R¹² in L is —C≡N or —COOH (the latter being possible for aryls/heteroaryls only) and all the others R¹² in L are H. In alternative embodiments, for aryls/heteroaryls only, preferably when L is a chain of 5 atoms in length, one R¹² on a first carbon atom in L is —C≡N, one R¹² on a second carbon atom in L is -T-COOR¹⁴, and all others R¹² in L are H.

In preferred embodiments, R¹¹ independently represents H or alkyl, alkenyl, alkynyl, alkenynyl, preferably H or alkyl (preferably methyl), and more preferably H.

In preferred embodiments, T is a covalent bond. In preferred embodiments, R¹⁴ is H.

In preferred embodiments, —C(R¹²)₂—C(═O)—N(R¹¹)— represents —CH₂—C(═O)—N(R¹¹)—, or —CH(CN)—C(═O)—N(R¹¹)—.

In most preferred embodiments:
—C(R¹²)₂—N(R¹¹)— represents —CH₂—NH—,
—C(R¹²)₂—C(═O)—N(R¹¹)— represents —CH₂—C(═O)—NH— or —CH(CN)—C(═O)—NH,
—C(R¹²)₂—C(R¹²)₂—N(R¹¹)—C(═O)— represents —CH₂—CH₂—N(H)—C(═O)—,
—C(R¹²)₂—C(═O)—N(R¹¹)—C(R¹²)₂— represents —CH₂—C(═O)—N(alkyl)-CH(alkyl)- (in which both alkyl are preferably C₁₋₆ alkyl, more preferably methyl), and/or
for aryls/heteroaryls only, —C(R¹²)₂—C(═O)—N(R¹¹)—C(R¹²)₂—C(R¹²)₂—
represents —CH₂—C(═O)—NH—CH₂—CH₂— or —CH(C≡N)—C(═O)—NH—CH(—COOH)—CH₂—.

In preferred embodiments, R¹⁰ represents:
a 6-membered cycle selected from heterocycloalkyl, heterocycloalkenyl, heterocycloalkynyl, or heterocycloalkenynyl, each of which comprising one or more nitrogen ring atoms as sole heteroatoms, and each of which being independently unsubstituted or substituted as described above
or
aryl or heteroaryl, each of which independently unsubstituted or substituted as described above,
   and preferably
a 6-membered cycle that is a heterocycloalkyl comprising one or more nitrogen ring atoms as sole heteroatoms, being unsubstituted or substituted as described above,
or
aryl or heteroaryl, each of which independently unsubstituted or substituted as described above.

Preferred heterocycloalkyls include piperidinyl (preferably piperidin-4-yl) and piperazinyl (preferably piperazin-1-yl), each of which independently unsubstituted or substituted as described above. Preferred aryls include phenyl, unsubstituted or substituted as described above. Preferred heteroaryls include pyridinyl (preferably pyridin-3-yl or pyridin-4-yl) and pyrimidinyl (preferably pyrimidin-2-yl), each of which independently unsubstituted or substituted as described above.

In embodiments, wherein R¹⁰ is a 6-membered cycle, this cycle is unsubstituted or substituted with alkyl, preferably C₁₋₆ alkyl, more preferably C₁₋₃ alkyl, preferably methyl, ethyl or isopropryl.

In embodiments, the 6-membered cycle is substituted with one or two substituents. In embodiments, the substituent(s) (if any) on the 6-membered cycle is(are) located in ortho and/or para from the point of attachment to L. In embodiments in which the 6-membered cycle is a heterocycle, the substituent is located on a nitrogen atom of the heterocycle, preferably in para from the point of attachment to L.

In embodiments, the aryl is unsubstituted or substituted. In embodiments the aryl is unsubstituted or substituted with one substituent. When the aryl is a phenyl, the substituent(s) (if any) is(are) located in meta and/or para (preferably meta) (or alternatively preferably para) of the point of attachment to L. In embodiments the aryl is unsubstituted or substituted with one or more, preferably one, of:
—N(R¹⁵)—C(═O)—R¹⁵, preferably —NH—C(═O)—R¹⁵, preferably wherein R¹⁵ is alkyl (preferably methyl) unsubstituted or substituted with one or more (preferably one) amino, alkylamino, dialkylamino, preferably dialkylamino, preferably wherein the alkyl (attached to the N atom) is C₁₋₆ alkyl, preferably C₁₋₃ alkyl, preferably methyl,
—OR¹⁸, or preferably wherein R¹⁸ is H or alkyl unsubstituted or substituted with one or more (preferably one) amino, alkylamino, dialkylamino, preferably dialkylamino, preferably wherein the alkyl is C₁₋₆ alkyl, preferably C₁₋₃ alkyl, preferably methyl.
alkyl-N(R¹⁵)₂, preferably wherein the alkyl is C₁₋₆ alkyl, preferably C₁₋₃ alkyl, preferably methyl; preferably wherein R¹⁵ is alkyl, preferably C₁₋₆ alkyl, preferably C₁₋₃ alkyl, preferably methyl.

In embodiments, the heteroaryl is unsubstituted.

Non-limiting examples of compounds of embodiments A) that may or may not fall within one or more formula (II), (III), and/or (IV) below, include:

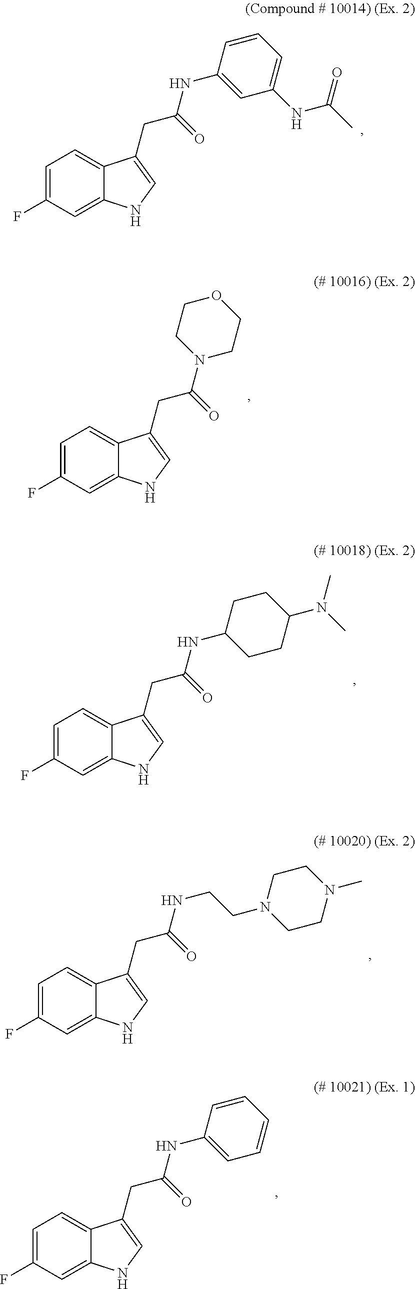 [SMILES: CC(=O)Nc1cccc(NC(=O)Cc2c[nH]c3cc(F)ccc23)c1 ; O=C(Cc1c[nH]c2cc(F)ccc12)N1CCOCC1 ; CN(C)C1CCC(NC(=O)Cc2c[nH]c3cc(F)ccc23)CC1 ; CN1CCN(CCNC(=O)Cc2c[nH]c3cc(F)ccc23)CC1 ; O=C(Cc1c[nH]c2cc(F)ccc12)Nc1ccccc1]

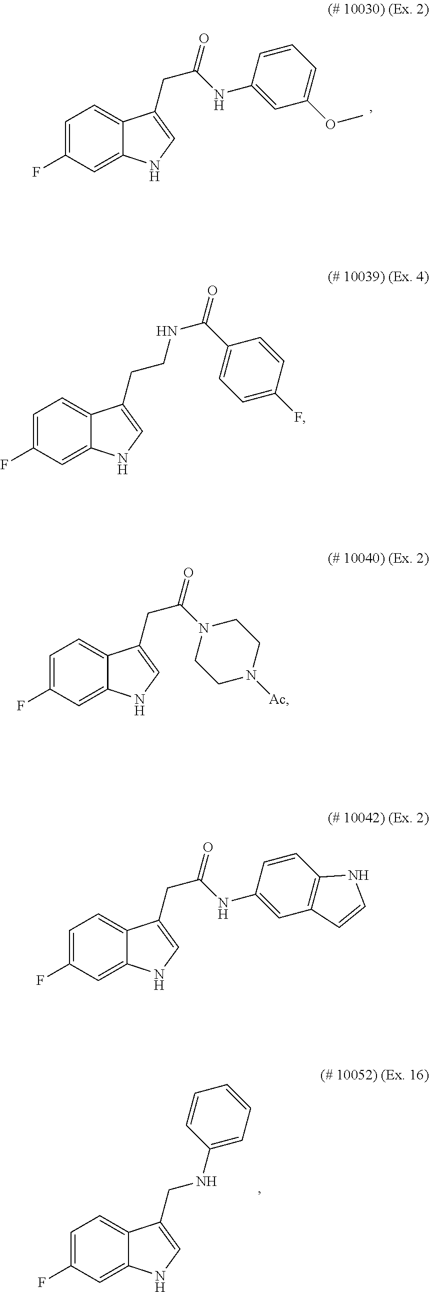 [SMILES: COc1cccc(NC(=O)Cc2c[nH]c3cc(F)ccc23)c1 ; O=C(NCCc1c[nH]c2cc(F)ccc12)c1ccc(F)cc1 ; CC(=O)N1CCN(C(=O)Cc2c[nH]c3cc(F)ccc23)CC1 ; O=C(Cc1c[nH]c2cc(F)ccc12)Nc1ccc2[nH]ccc2c1 ; Fc1ccc2c(CNc3ccccc3)c[nH]c2c1]

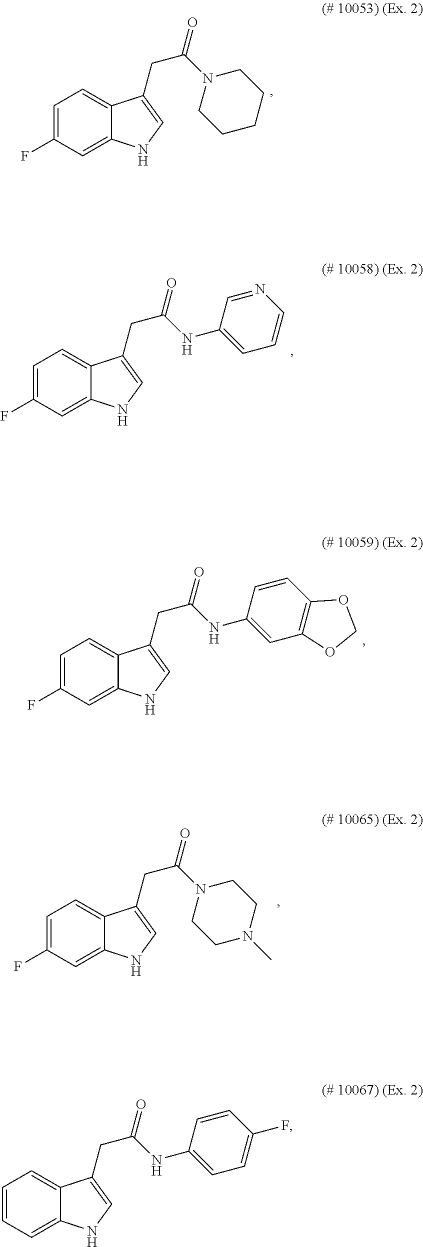 [SMILES: O=C(Cc1c[nH]c2cc(F)ccc12)N1CCCCC1 ; O=C(Cc1c[nH]c2cc(F)ccc12)Nc1cccnc1 ; O=C(Cc1c[nH]c2cc(F)ccc12)Nc1ccc2c(c1)OCO2 ; CN1CCN(C(=O)Cc2c[nH]c3cc(F)ccc23)CC1 ; O=C(Cc1c[nH]c2ccccc12)Nc1ccc(F)cc1]

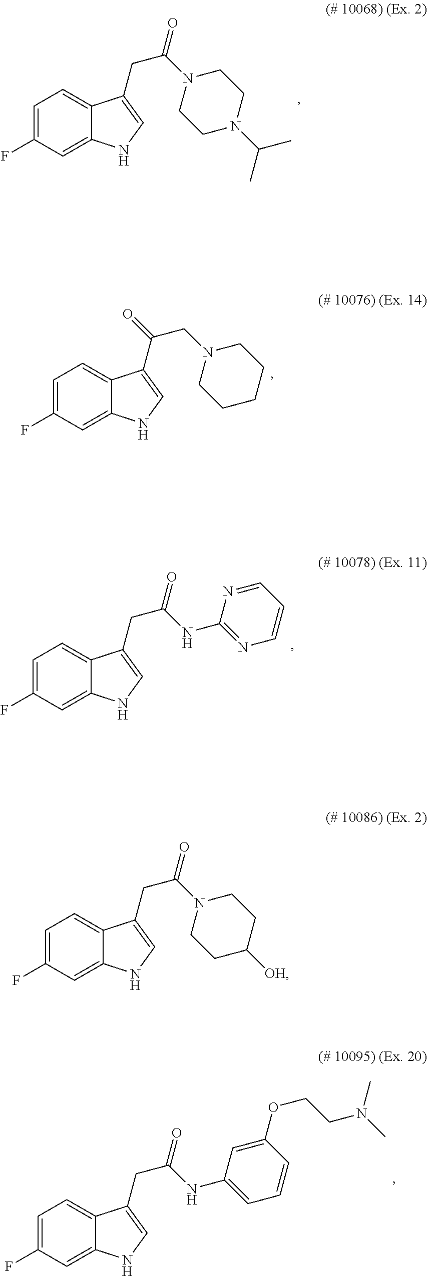 [SMILES: CC(C)N1CCN(C(=O)Cc2c[nH]c3cc(F)ccc23)CC1 ; O=C(CN1CCCCC1)c1c[nH]c2cc(F)ccc12 ; O=C(Cc1c[nH]c2cc(F)ccc12)Nc1ncccn1 ; O=C(Cc1c[nH]c2cc(F)ccc12)N1CCC(O)CC1 ; CN(C)CCOc1cccc(NC(=O)Cc2c[nH]c3cc(F)ccc23)c1]

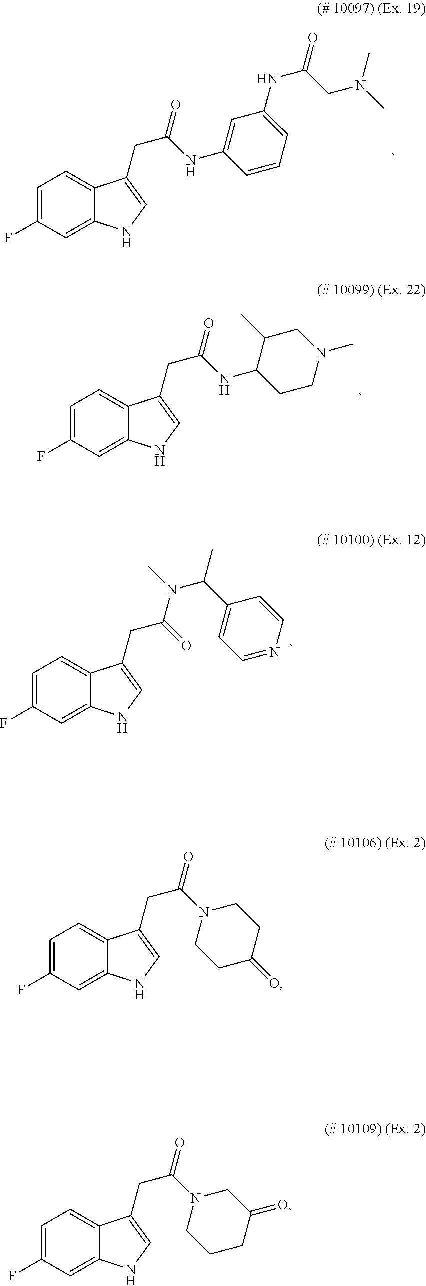 [SMILES: CN(C)CC(=O)Nc1cccc(NC(=O)Cc2c[nH]c3cc(F)ccc23)c1 ; CC1CN(C)CCC1NC(=O)Cc1c[nH]c2cc(F)ccc12 ; CC(c1ccncc1)N(C)C(=O)Cc1c[nH]c2cc(F)ccc12 ; O=C1CCN(C(=O)Cc2c[nH]c3cc(F)ccc23)CC1 ; O=C1CCCN(C(=O)Cc2c[nH]c3cc(F)ccc23)C1]

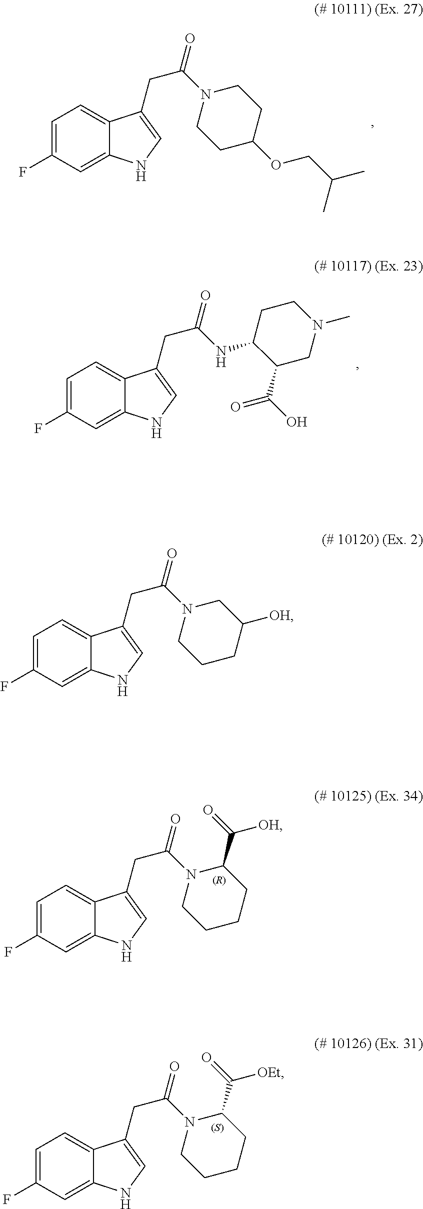

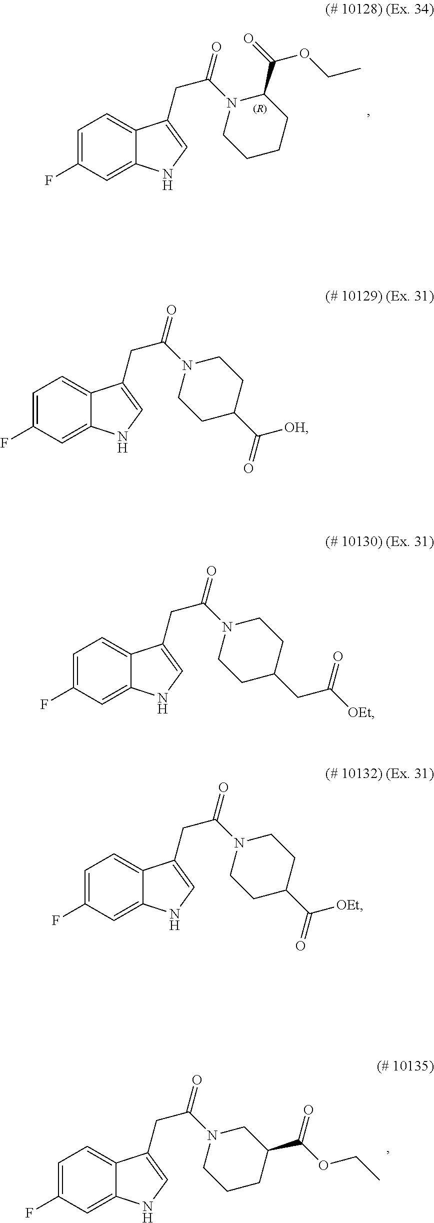

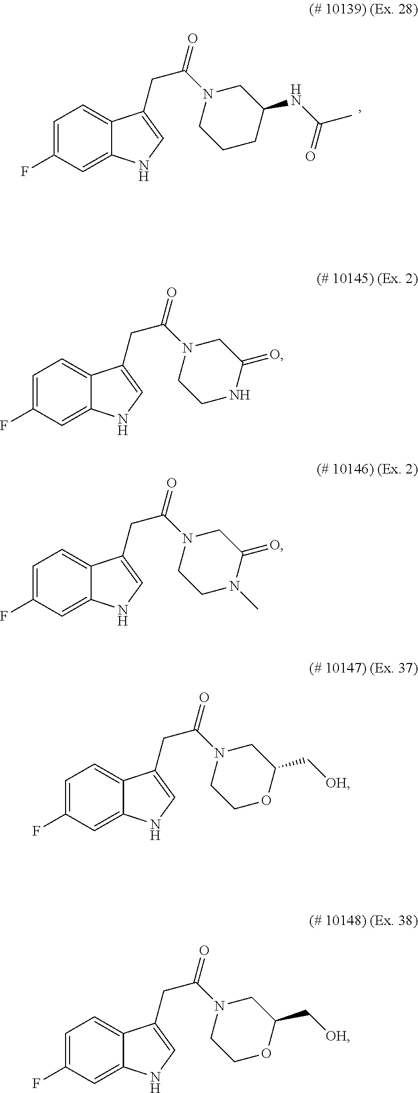 [SMILES: CC(=O)N[C@H]1CCCN(C(=O)Cc2c[nH]c3cc(F)ccc23)C1 ; O=C1CN(C(=O)Cc2c[nH]c3cc(F)ccc23)CCN1 ; CN1CCN(C(=O)Cc2c[nH]c3cc(F)ccc23)CC1=O ; O=C(Cc1c[nH]c2cc(F)ccc12)N1CCO[C@@H](CO)C1 ; O=C(Cc1c[nH]c2cc(F)ccc12)N1CCO[C@H](CO)C1]

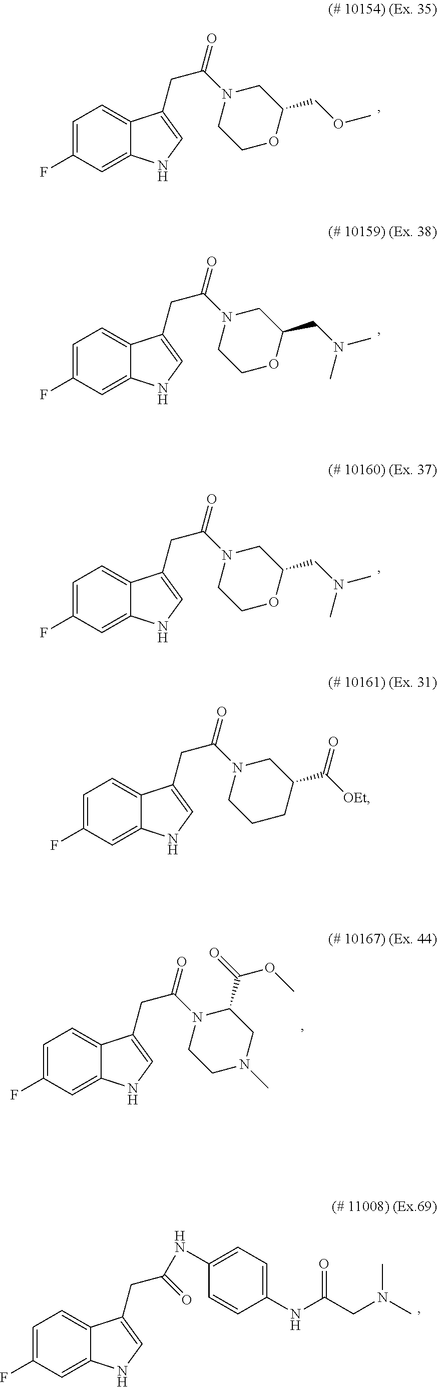 [SMILES: COC[C@H]1CN(C(=O)Cc2c[nH]c3cc(F)ccc23)CCO1 ; CN(C)C[C@@H]1CN(C(=O)Cc2c[nH]c3cc(F)ccc23)CCO1 ; CN(C)C[C@H]1CN(C(=O)Cc2c[nH]c3cc(F)ccc23)CCO1 ; CCOC(=O)[C@@H]1CCCN(C(=O)Cc2c[nH]c3cc(F)ccc23)C1 ; COC(=O)[C@@H]1CN(C)CCN1C(=O)Cc1c[nH]c2cc(F)ccc12 ; CN(C)CC(=O)Nc1ccc(NC(=O)Cc2c[nH]c3cc(F)ccc23)cc1]

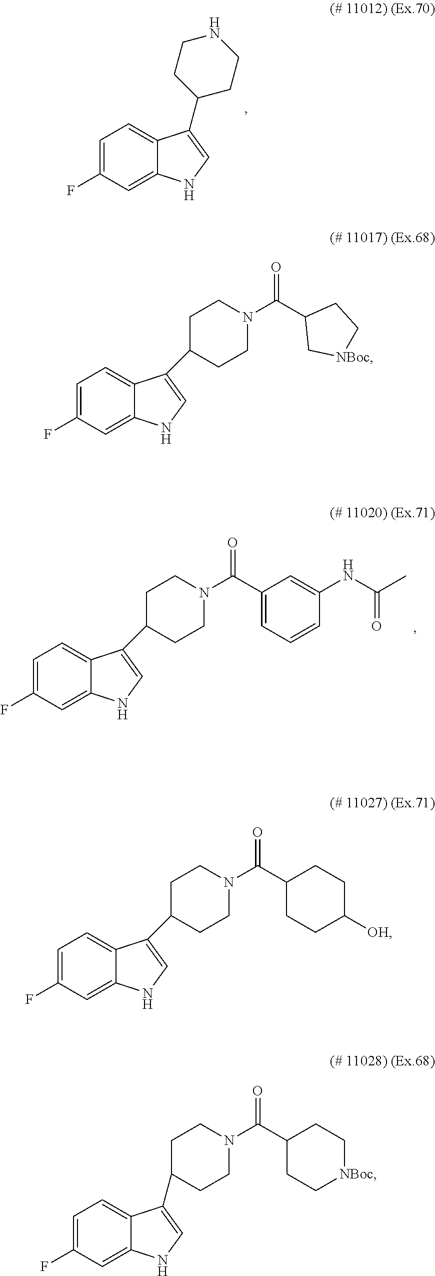 [SMILES: Fc1ccc2c(C3CCNCC3)c[nH]c2c1 ; CC(C)(C)OC(=O)N1CCC(C(=O)N2CCC(c3c[nH]c4cc(F)ccc34)CC2)C1 ; CC(=O)Nc1cccc(C(=O)N2CCC(c3c[nH]c4cc(F)ccc34)CC2)c1 ; O=C(C1CCC(O)CC1)N1CCC(c2c[nH]c3cc(F)ccc23)CC1 ; CC(C)(C)OC(=O)N1CCC(C(=O)N2CCC(c3c[nH]c4cc(F)ccc34)CC2)CC1]

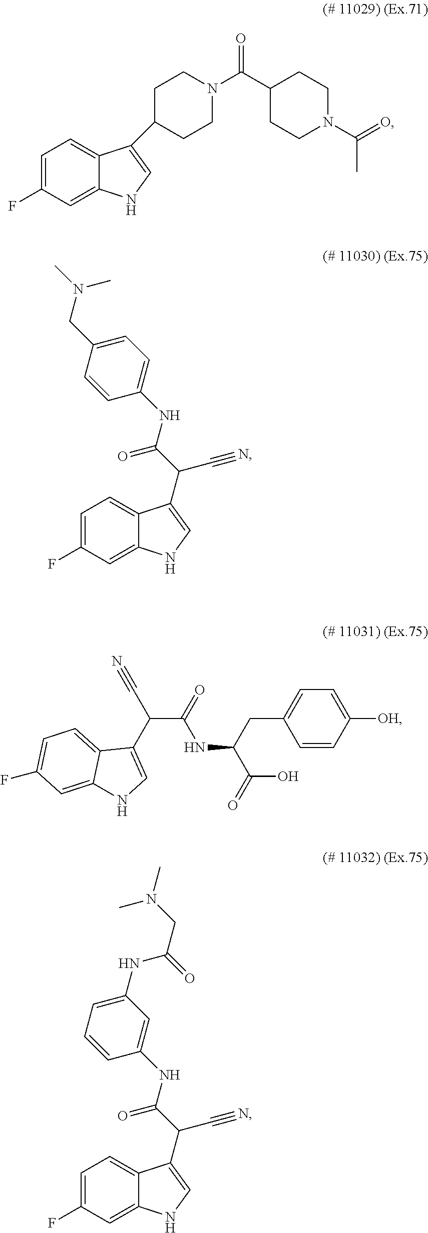 [SMILES: CC(=O)N1CCC(C(=O)N2CCC(c3c[nH]c4cc(F)ccc34)CC2)CC1 ; CN(C)Cc1ccc(NC(=O)C(C#N)c2c[nH]c3cc(F)ccc23)cc1 ; N#CC(C(=O)N[C@@H](Cc1ccc(O)cc1)C(=O)O)c1c[nH]c2cc(F)ccc12 ; CN(C)CC(=O)Nc1cccc(NC(=O)C(C#N)c2c[nH]c3cc(F)ccc23)c1]

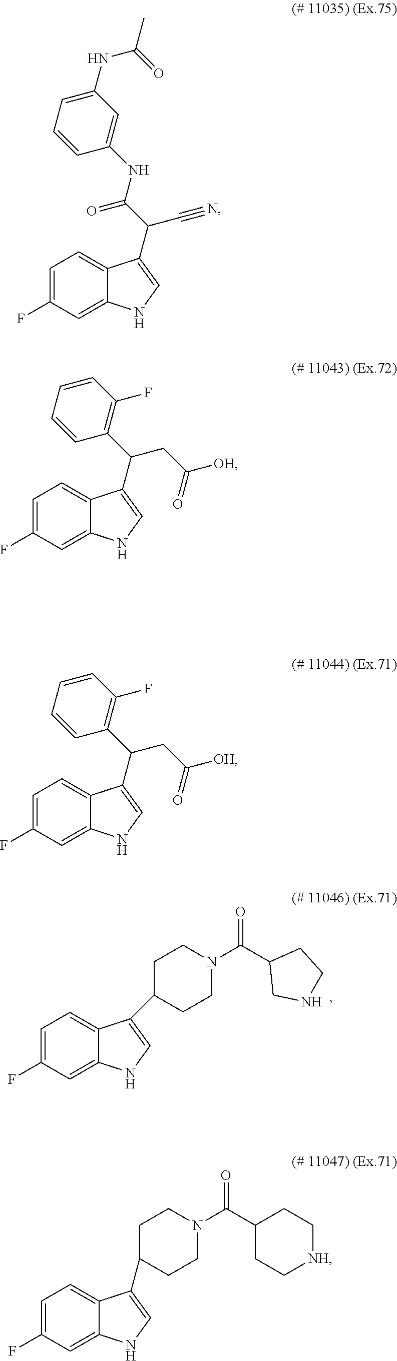 [SMILES: CC(=O)Nc1cccc(NC(=O)C(C#N)c2c[nH]c3cc(F)ccc23)c1 ; O=C(O)CC(c1ccccc1F)c1c[nH]c2cc(F)ccc12 ; O=C(O)CC(c1ccccc1F)c1c[nH]c2cc(F)ccc12 ; O=C(C1CCNC1)N1CCC(c2c[nH]c3cc(F)ccc23)CC1 ; O=C(C1CCNCC1)N1CCC(c2c[nH]c3cc(F)ccc23)CC1]

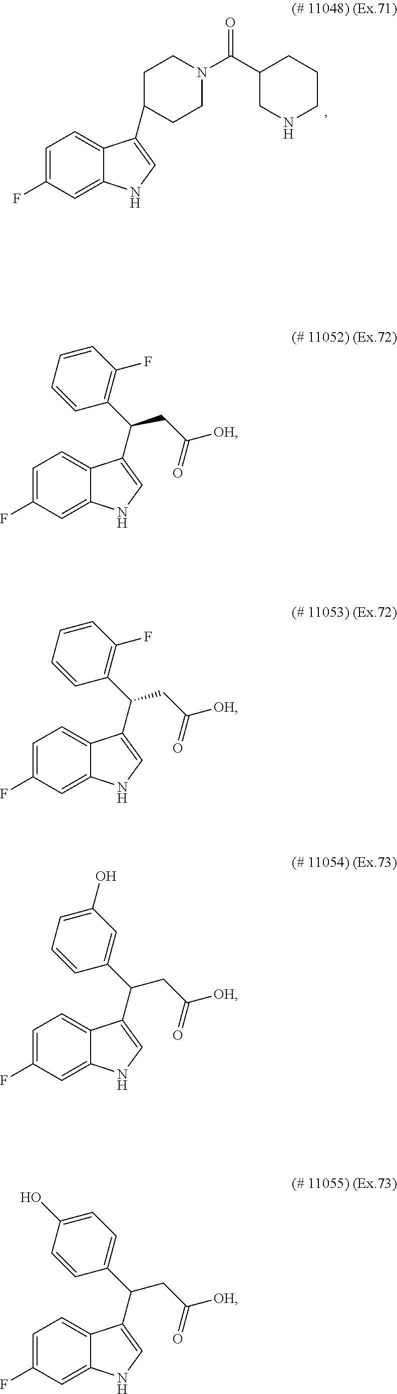 [SMILES: O=C(C1CCCNC1)N1CCC(c2c[nH]c3cc(F)ccc23)CC1 ; O=C(O)C[C@H](c1ccccc1F)c1c[nH]c2cc(F)ccc12 ; O=C(O)C[C@@H](c1ccccc1F)c1c[nH]c2cc(F)ccc12 ; O=C(O)CC(c1cccc(O)c1)c1c[nH]c2cc(F)ccc12 ; O=C(O)CC(c1ccc(O)cc1)c1c[nH]c2cc(F)ccc12]

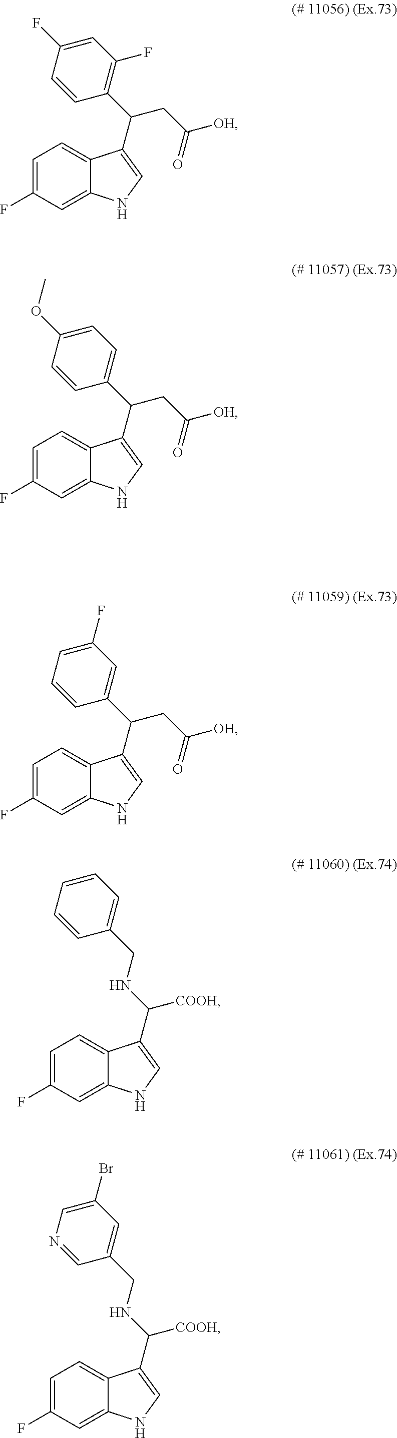 [SMILES: O=C(O)CC(c1ccc(F)cc1F)c1c[nH]c2cc(F)ccc12 ; COc1ccc(C(CC(=O)O)c2c[nH]c3cc(F)ccc23)cc1 ; O=C(O)CC(c1cccc(F)c1)c1c[nH]c2cc(F)ccc12 ; O=C(O)C(NCc1ccccc1)c1c[nH]c2cc(F)ccc12 ; O=C(O)C(NCc1cncc(Br)c1)c1c[nH]c2cc(F)ccc12]

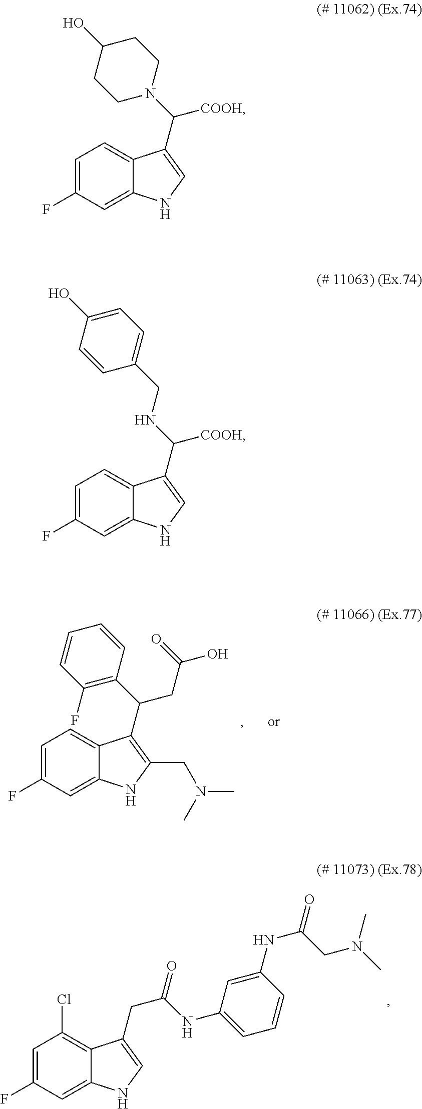 [SMILES: O=C(O)C(c1c[nH]c2cc(F)ccc12)N1CCC(O)CC1 ; O=C(O)C(NCc1ccc(O)cc1)c1c[nH]c2cc(F)ccc12 ; CN(C)Cc1[nH]c2cc(F)ccc2c1C(CC(=O)O)c1ccccc1F ; CN(C)CC(=O)Nc1cccc(NC(=O)Cc2c[nH]c3cc(F)cc(Cl)c23)c1]

or a pharmaceutically acceptable salt, ester, solvate, isomer, or tautomer thereof.

Preferred compounds include Compounds #10014, 10018, 10040, 10053, 10076, 10086, 10095, 10097, 10159, 11008, 11032, 11043, 11052, 11053, 11054, 11055, 11066, and 11073 and pharmaceutically acceptable salts, esters, solvates, isomers, or tautomers thereof.

More preferred compounds include Compounds #10014, 10095, 10097, 11008, 11043, 11052, 11053, 11054, 11055, 11066, and 11073, and pharmaceutically acceptable salts, esters, solvates, isomers, or tautomers thereof.

Alternatively, more preferred compounds include Compounds #10018, 10040, 10086, 10095, 10097, 10032, 11053, and 11066, and pharmaceutically acceptable salts, esters, solvates, isomers, or tautomers thereof.

Yet more preferred compounds include Compounds #10095, 10097, 11032, 11053, and 11073, and pharmaceutically acceptable salts, esters, solvates, isomers, or tautomers thereof.

Most preferred compounds with HRAS activity include 11032, 11095, 11097, and pharmaceutically acceptable salts, esters, solvates, isomers, or tautomers thereof.

Most preferred compounds with KRAS activity include 10018, 10040, 10086, and 11066, and pharmaceutically acceptable salts, esters, solvates, isomers, or tautomers thereof.

Compounds with B) R¹⁰═COOH

As noted above, in some compounds, R¹⁰ represents —COOH.

In preferred embodiments, G represents —C(R⁴)═, preferably wherein R⁴ represents a halogen atom, preferably F.

In embodiments, L represents a chain that is at most 2 atoms in length, preferably only 1 atom in length.

In embodiments, L represents —C(R¹²)₂— or —C(R¹²)₂—C(R¹²)₂—.

In embodiments, all R¹² represent H.

In alternative embodiments, at least one (preferably one) R¹² represents:
—C≡N, —N(R¹⁵)₂, —C(═O)—R¹⁵, or
alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰
preferably —N(R¹⁵)₂, —C(═O)—R¹⁵, or
cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰
more preferably —N(R¹⁵)₂, —C(═O)—R¹⁵, or
heterocycloalkyl, heterocycloalkenyl, heterocycloalkynyl, heterocycloalkenynyl, aryl, or heteroaryl, each of which independently unsubstituted or substituted with one or more R³⁰, or
yet more preferably —N(R¹⁵)₂, —C(═O)—R¹⁵, or
heterocycloalkyl or aryl, each of which independently unsubstituted or substituted with one or more R³⁰ and preferably any other R¹² represent H. Preferred heterocycloalkyls include piperidinyl (preferably piperidin-1-yl). Preferred aryls include phenyl.

In embodiments, the above groups are substituted with one or two R³⁰, preferably only one R³⁰.

In embodiments, each R³⁰ independently is:
a halogen atom, —OR¹⁵, or
alkyl, alkenyl, alkynyl, alkenynyl, each of which independently unsubstituted or substituted with one or more R¹⁶, preferably unsubstituted,
preferably a halogen atom, —OR¹⁵, or alkyl unsubstituted or substituted with one or more R¹⁶, preferably unsubstituted.
   Preferred halogen atoms include fluoride and bromide. Preferred alkyls include C₁₋₆ alkyls, preferably methyl or ethyl.

In embodiments, R¹⁵ represents H or alkyl, alkenyl, alkynyl, or alkenynyl, each of which independently unsubstituted or substituted with one or more R¹⁶, preferably unsubstituted. Preferably R¹⁵ represents H or alkyl unsubstituted or substituted with one or more R¹⁶, preferably unsubstituted. Preferred alkyls include C₁₋₆ alkyls, preferably methyl.

Non-limiting examples of compounds of embodiments B) that may or may not fall within one or more formula (II), (III), and/or (IV) below, include

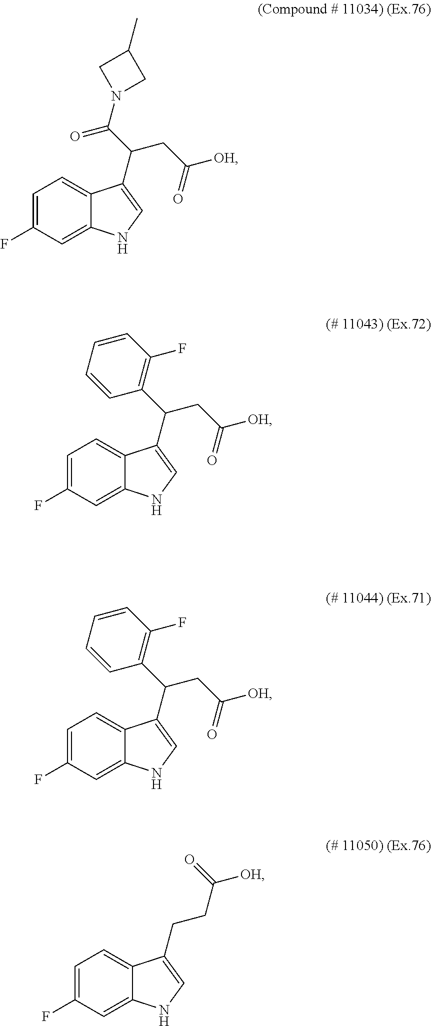 [SMILES: CC1CN(C(=O)C(CC(=O)O)c2c[nH]c3cc(F)ccc23)C1 ; O=C(O)CC(c1ccccc1F)c1c[nH]c2cc(F)ccc12 ; O=C(O)CC(c1ccccc1F)c1c[nH]c2cc(F)ccc12 ; O=C(O)CCc1c[nH]c2cc(F)ccc12]

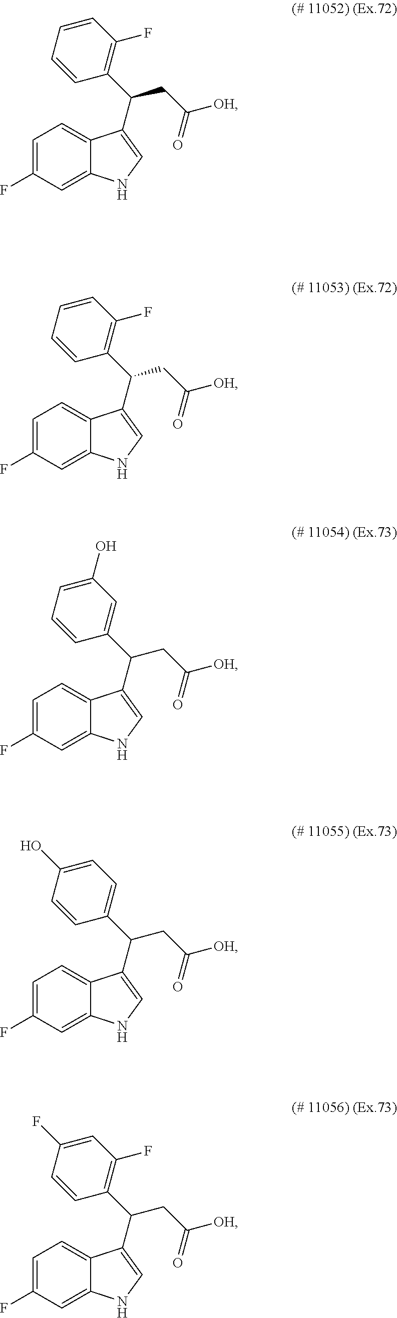 [SMILES: O=C(O)C[C@H](c1ccccc1F)c1c[nH]c2cc(F)ccc12 ; O=C(O)C[C@@H](c1ccccc1F)c1c[nH]c2cc(F)ccc12 ; O=C(O)CC(c1cccc(O)c1)c1c[nH]c2cc(F)ccc12 ; O=C(O)CC(c1ccc(O)cc1)c1c[nH]c2cc(F)ccc12 ; O=C(O)CC(c1ccc(F)cc1F)c1c[nH]c2cc(F)ccc12]

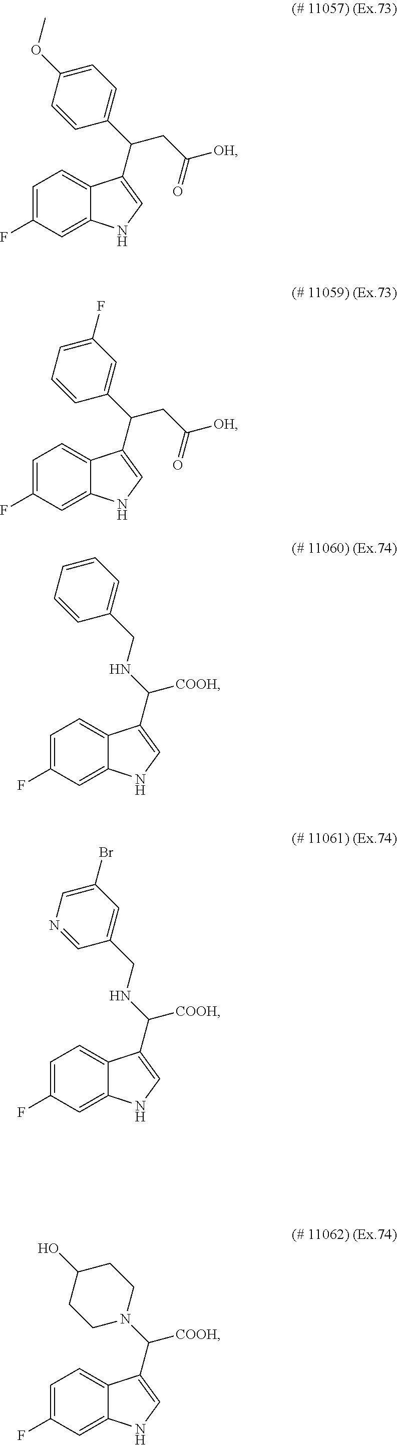 [SMILES: COc1ccc(C(CC(=O)O)c2c[nH]c3cc(F)ccc23)cc1 ; O=C(O)CC(c1cccc(F)c1)c1c[nH]c2cc(F)ccc12 ; O=C(O)C(NCc1ccccc1)c1c[nH]c2cc(F)ccc12 ; O=C(O)C(NCc1cncc(Br)c1)c1c[nH]c2cc(F)ccc12 ; O=C(O)C(c1c[nH]c2cc(F)ccc12)N1CCC(O)CC1]

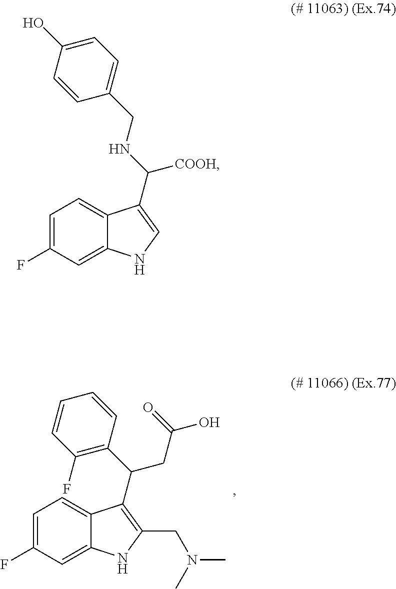 [SMILES: O=C(O)C(NCc1ccc(O)cc1)c1c[nH]c2cc(F)ccc12 ; CN(C)Cc1[nH]c2cc(F)ccc2c1C(CC(=O)O)c1ccccc1F]

or a pharmaceutically acceptable salt, ester, solvate, isomer, or tautomer thereof.

Most preferred compounds include 11066.

Compounds with C) R¹⁰ and R¹¹ Forming a Heterocycle

As noted above, in embodiments C), R¹⁰ is attached to a nitrogen atom of a —N(R¹¹)— group, and R¹⁰ and R¹¹ together with the nitrogen atom to which they are attached form a heteroaryl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl.

In preferred embodiments, L comprises at least one —C(═O)— and a least one-N(R¹¹)—. In most preferred embodiments, L comprises exactly one —C(═O)— and exactly one-N(R¹¹)—.

As noted above, L ends with a —N(R¹¹)— group to which R₁₀ is attached. This means that the L chain atom closest to —R¹⁰ is the nitrogen atom of this —N(R¹¹)— group.

In more preferred embodiments, -L- ends with a —C(═O)—N(R¹¹)— group.

In yet more preferred embodiments, L represents —C(R¹²)₂—C(═O)—N(R¹¹)— or —C(═O)—C(R¹²)₂—N(R¹¹)—, preferably —C(R¹²)₂—C(═O)—N(R¹¹)—.

In preferred embodiments, each R¹² independently represents H, —C≡N, or alkyl (preferably methyl or ethyl).

In preferred embodiments, a first R¹² on a carbon atom represents alkyl (preferably methyl or ethyl), or —C≡N, and a second R¹² on said carbon atom represent H. In alternative embodiments, the two R¹² on a carbon atom represent alkyl.

In alternative preferred embodiments, each R¹² represents H.

In embodiments, any other R¹¹ represents H.

In preferred embodiments, R¹⁰ and R¹¹ together with the nitrogen atom to which they are attached form a heterocycloalkyl. In more preferred embodiments, the heterocycloalkyl is azetidin-1-yl, pyrrolidi-1-nyl, piperidin-1-yl, piperidinon-1-yl (preferably piperidin-3-on-1-yl or piperidin-4-on-1-yl), morpholin-1-yl, piperazin-1-yl, piperazinone-1-yl (more preferably piperazin-3-on-1-yl),

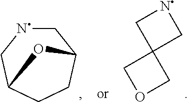

In most preferred embodiments, the heterocycloalkyl is azetidin-1-yl, piperidin-1-yl, or morpholin-1-yl.

In preferred embodiments, the heteroaryl, heterocycloalkyl, heterocycloalkenyl, heterocycloalkynyl and heterocycloalkenynyl formed by R¹⁰ and R¹¹ together with the nitrogen atom to which they are attached is unsubstituted or substituted with one or more (preferably one or two, more preferably one) of alkyl, —OR¹⁵, —C(═O)—N(R¹⁵)₂, —N(R¹⁵)—C(═O)—R¹⁵, or —C(═O)—OR²⁰,

more preferably unsubstituted or substituted with one or more (preferably one) of:
alkyl (preferably C₁₋₆, more preferably methyl or ethyl),
OH,
alkoxy (preferably C₁₋₆, more preferably methoxy, ethoxy, propoxy (preferably isopropoxy), or butoxy (preferably isobutoxy)),
—CONH₂,
═NH—C(═O)-alkyl (preferably C₁₋₆, more preferably ═NH—C(═O)—CH₃),
—C(═O)O-alkyl (preferably C₁₋₆, more preferably —C(═O)O—CH₂CH₃),
   and most preferably unsubstituted or substituted with one or more, preferably one or two, more preferably one of alkyl, hydroxyl, or —CH₂—N(CH₃)₂.

In preferred embodiments, the R¹⁵ of the OR¹⁵ group, the alkoxy and/or alkyl substituting the cycle formed by R¹⁰ and R¹¹ are unsubstituted or substituted with one or more of alkoxy (preferably C₁₋₆, more preferably methoxy), alkoxycarbonyl (preferably C₁₋₆, more preferably —C(═O)OCH₂CH₃), hydroxyl, amino, alkylamino, or dialkylamino (preferably C₁₋₆, more preferably —N(CH₃)₂).

When R¹⁰ and R¹¹ together with the nitrogen atom to which they are attached form azetidin-1-yl unsubstituted or substituted as described above, the azetidinyl is preferably unsubstituted or substituted at position 3.

When R¹⁰ and R¹¹ together with the nitrogen atom to which they are attached form pyrrolidi-1-nyl unsubstituted or substituted as described above, the pyrrolidi-1-nyl is preferably unsubstituted or substituted at position 3.

When R¹⁰ and R¹¹ together with the nitrogen atom to which they are attached form piperidin-1-yl unsubstituted or substituted as described above, the piperidin-1-yl is preferably unsubstituted or substituted at position, 2, 3, or 4, preferably position 2 or 4, more preferably position 4.

When R¹⁰ and R¹¹ together with the nitrogen atom to which they are attached form morpholin-1-yl unsubstituted or substituted as described above, the morpholin-1-yl is preferably unsubstituted or substituted at position 3.

When R¹⁰ and R¹¹ together with the nitrogen atom to which they are attached form piperazin-1-yl unsubstituted or substituted as described above, the piperazin-1-yl is preferably unsubstituted or substituted at position 2 or 4, preferably position 4 (i.e. on the nitrogen atom facing the nitrogen atom bearing R¹⁰ and R¹¹).

When R¹⁰ and R¹¹ together with the nitrogen atom to which they are attached form piperazinon-1-yl unsubstituted or substituted as described above, the piperazinon-1-yl is preferably unsubstituted or substituted at position 4 (i.e. on the nitrogen atom facing the nitrogen atom bearing R¹⁰ and R¹¹).

As noted above, in embodiment C), when R¹⁰ and R¹¹ together with the nitrogen atom to which they are attached form morpholinyl, the morpholinyl is free of a substituent containing a —C(═O)— group. In embodiments, R¹⁰ and R¹¹ together with the nitrogen atom to which they are attached do not form morpholinyl. In embodiments, R¹⁰ and R¹¹ together with the nitrogen atom to which they are attached do not form a heterocycle containing an oxygen ring atom.

As also noted above, in embodiment C), when one R¹² on a given carbon atom is methyl, any other R¹² on said given carbon atom is other than methyl. In embodiments, R¹² is not methyl.

Non-limiting examples of compounds C) that may or may not fall within one or more formula (II), (Ill), and/or (IV) below, include:

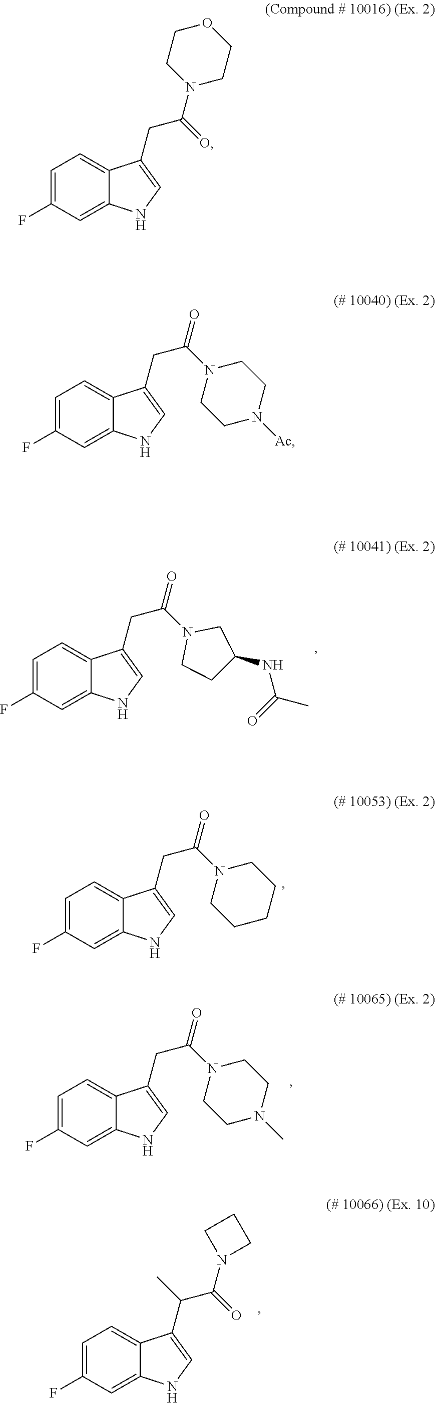 [SMILES: O=C(Cc1c[nH]c2cc(F)ccc12)N1CCOCC1 ; CC(=O)N1CCN(C(=O)Cc2c[nH]c3cc(F)ccc23)CC1 ; CC(=O)N[C@H]1CCN(C(=O)Cc2c[nH]c3cc(F)ccc23)C1 ; O=C(Cc1c[nH]c2cc(F)ccc12)N1CCCCC1 ; CN1CCN(C(=O)Cc2c[nH]c3cc(F)ccc23)CC1 ; CC(C(=O)N1CCC1)c1c[nH]c2cc(F)ccc12]

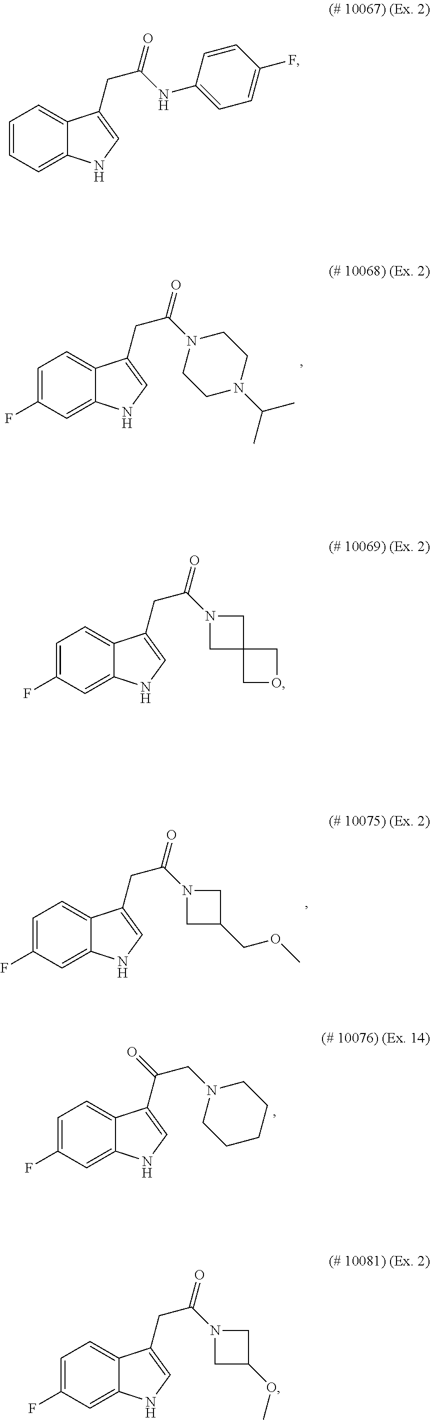 [SMILES: O=C(Cc1c[nH]c2ccccc12)Nc1ccc(F)cc1 ; CC(C)N1CCN(C(=O)Cc2c[nH]c3cc(F)ccc23)CC1 ; O=C(Cc1c[nH]c2cc(F)ccc12)N1CC2(COC2)C1 ; COCC1CN(C(=O)Cc2c[nH]c3cc(F)ccc23)C1 ; O=C(CN1CCCCC1)c1c[nH]c2cc(F)ccc12 ; COC1CN(C(=O)Cc2c[nH]c3cc(F)ccc23)C1]

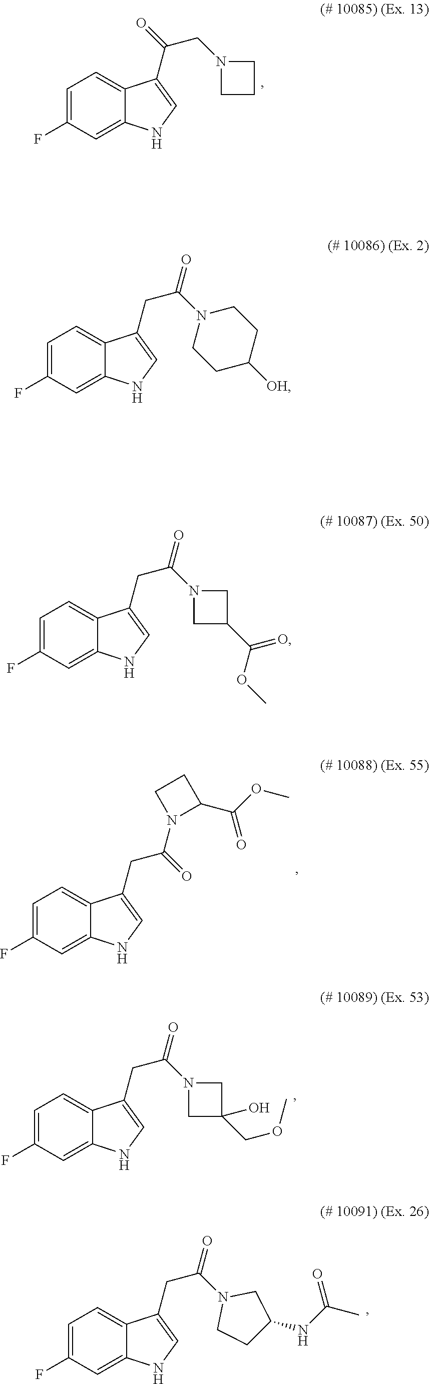 [SMILES: O=C(CN1CCC1)c1c[nH]c2cc(F)ccc12 ; O=C(Cc1c[nH]c2cc(F)ccc12)N1CCC(O)CC1 ; COC(=O)C1CN(C(=O)Cc2c[nH]c3cc(F)ccc23)C1 ; COC(=O)C1CCN1C(=O)Cc1c[nH]c2cc(F)ccc12 ; COCC1(O)CN(C(=O)Cc2c[nH]c3cc(F)ccc23)C1 ; CC(=O)N[C@@H]1CCN(C(=O)Cc2c[nH]c3cc(F)ccc23)C1]

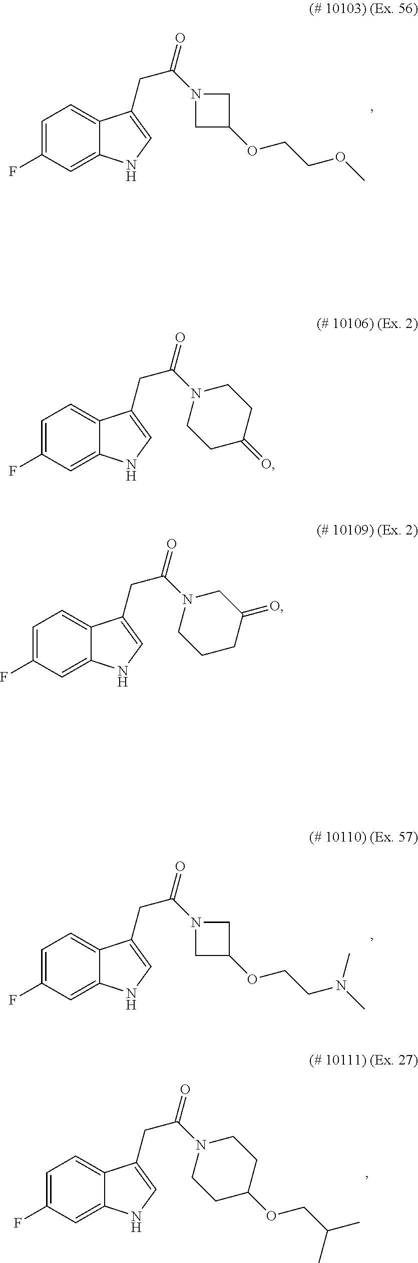 [SMILES: COCCOC1CN(C(=O)Cc2c[nH]c3cc(F)ccc23)C1 ; O=C1CCN(C(=O)Cc2c[nH]c3cc(F)ccc23)CC1 ; O=C1CCCN(C(=O)Cc2c[nH]c3cc(F)ccc23)C1 ; CN(C)CCOC1CN(C(=O)Cc2c[nH]c3cc(F)ccc23)C1 ; CC(C)COC1CCN(C(=O)Cc2c[nH]c3cc(F)ccc23)CC1]

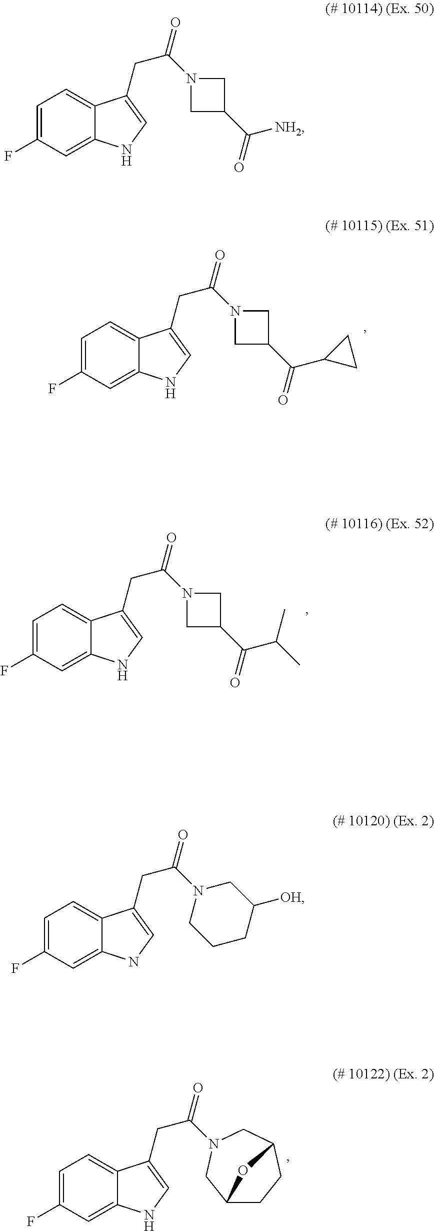 [SMILES: NC(=O)C1CN(C(=O)Cc2c[nH]c3cc(F)ccc23)C1 ; O=C(C1CC1)C1CN(C(=O)Cc2c[nH]c3cc(F)ccc23)C1 ; CC(C)C(=O)C1CN(C(=O)Cc2c[nH]c3cc(F)ccc23)C1 ; O=C(Cc1c[nH]c2cc(F)ccc12)N1CCCC(O)C1 ; O=C(Cc1c[nH]c2cc(F)ccc12)N1C[C@H]2CC[C@@H](C1)O2]

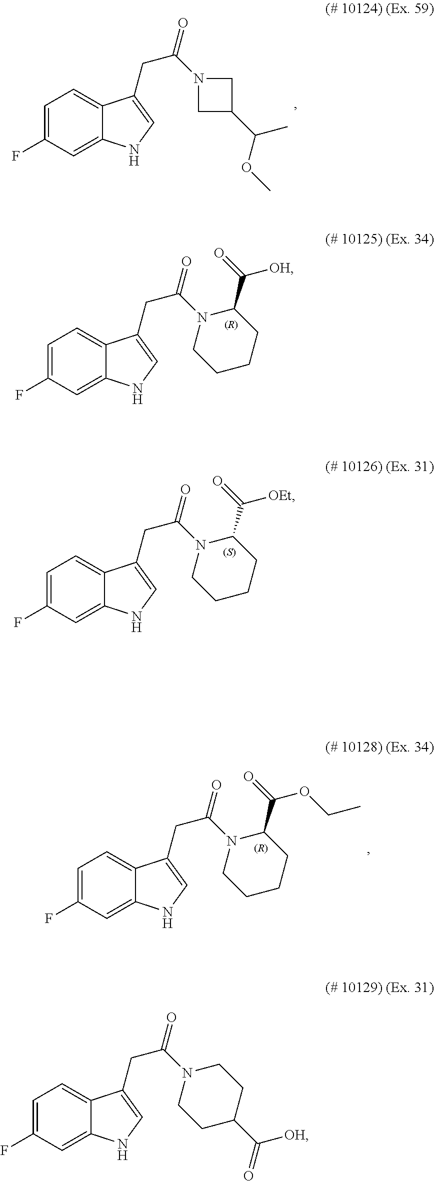

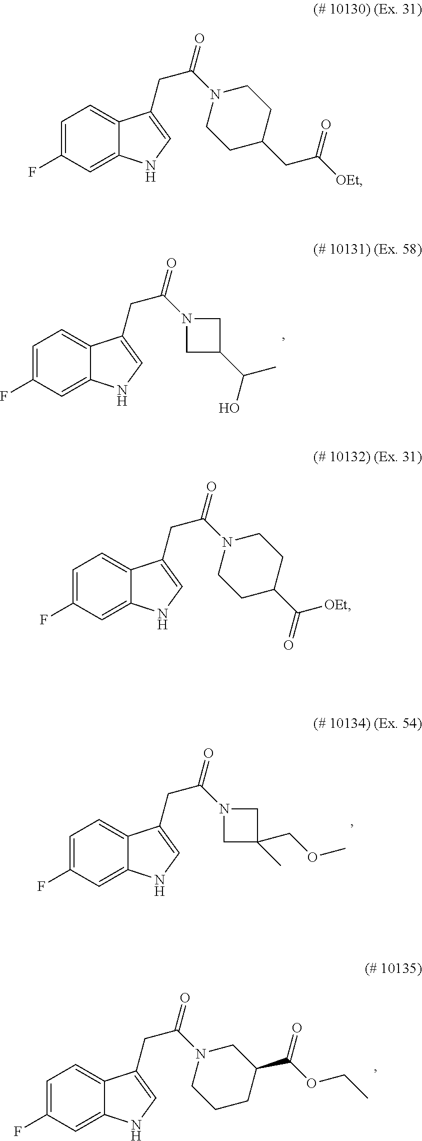 [SMILES: CCOC(=O)CC1CCN(C(=O)Cc2c[nH]c3cc(F)ccc23)CC1 ; CC(O)C1CN(C(=O)Cc2c[nH]c3cc(F)ccc23)C1 ; CCOC(=O)C1CCN(C(=O)Cc2c[nH]c3cc(F)ccc23)CC1 ; COCC1(C)CN(C(=O)Cc2c[nH]c3cc(F)ccc23)C1 ; CCOC(=O)[C@H]1CCCN(C(=O)Cc2c[nH]c3cc(F)ccc23)C1]

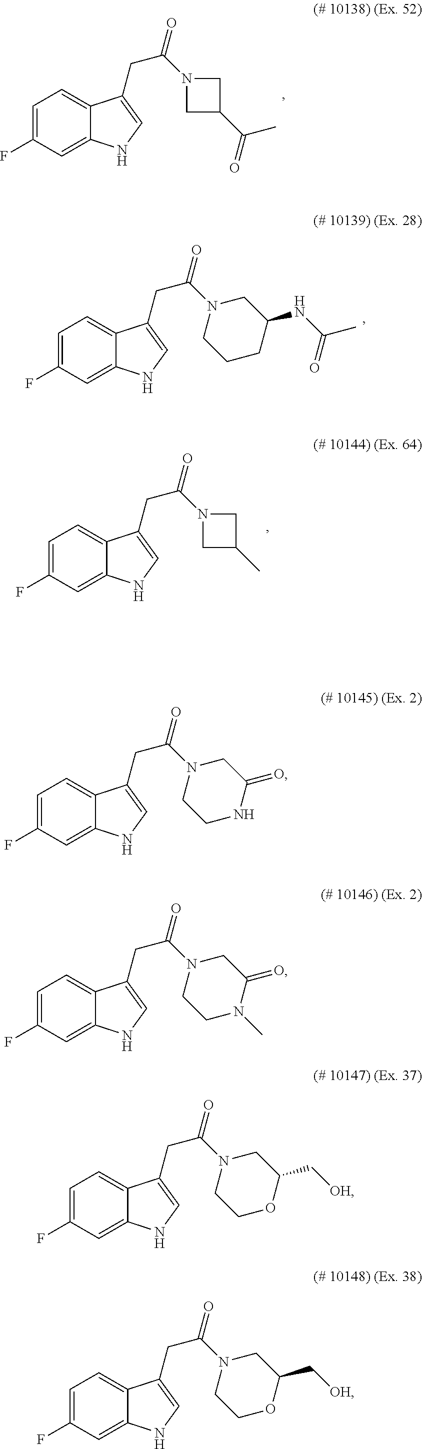

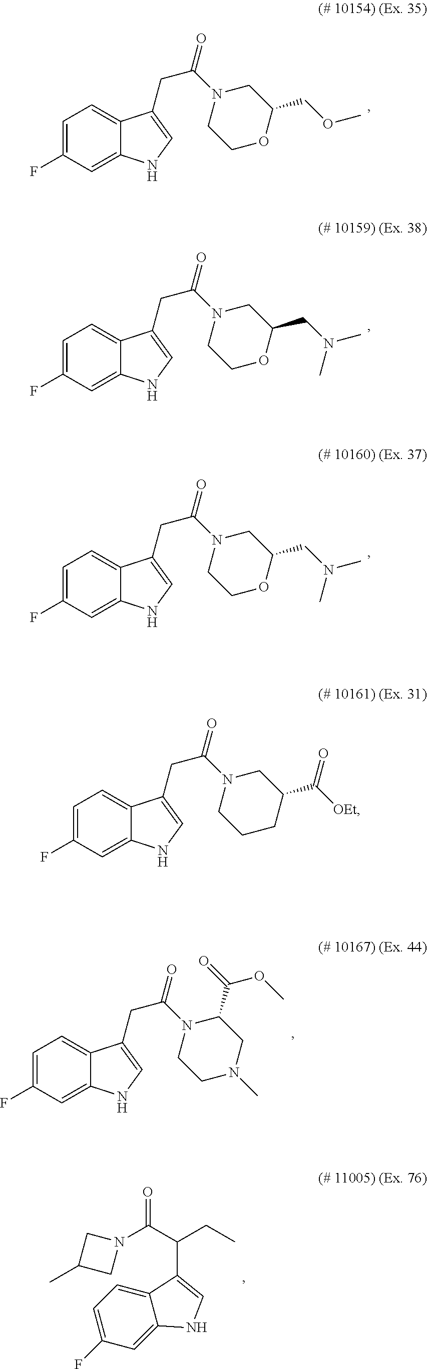

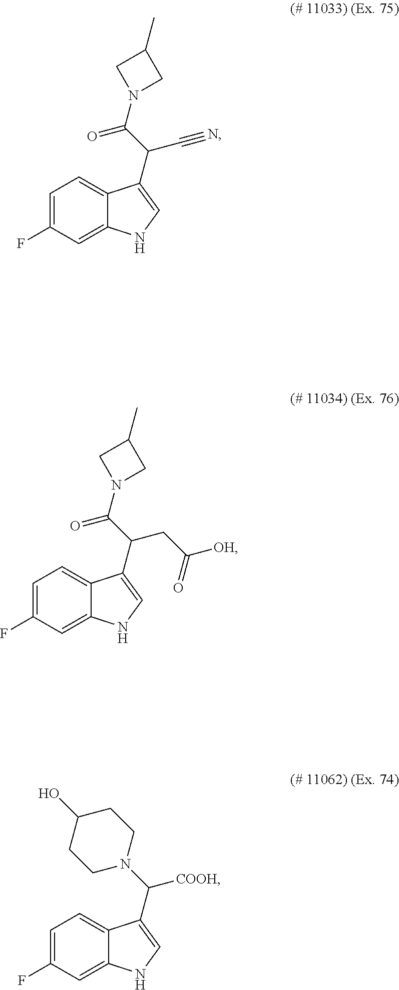 [SMILES: CC1CN(C(=O)C(C#N)c2c[nH]c3cc(F)ccc23)C1 ; CC1CN(C(=O)C(CC(=O)O)c2c[nH]c3cc(F)ccc23)C1 ; O=C(O)C(c1c[nH]c2cc(F)ccc12)N1CCC(O)CC1]

or a pharmaceutically acceptable salt, ester, solvate, isomer, or tautomer thereof.

More preferred compounds include Compounds #10040, 10053, 10066, 10076, 10086, 10159, and 11005 and pharmaceutically acceptable salts, esters, solvates, isomers, or tautomers thereof. Most preferred compounds include Compounds #10066 and 11005 and pharmaceutically acceptable salts, esters, solvates, isomers, or tautomers thereof.

Compounds with D) R¹¹ and R¹² Forming a Heterocycle

As noted above, in compounds of embodiments D), the R¹¹ of the —N(R¹¹)— group that ends the chain in L and one R¹² of said at least one —C(R¹²)₂— group together with the one or more atoms to which they are attached and any atom(s) between said one or more atoms form heterocycloalkyl, heterocycloalkanonyl, heterocycloalkenonyl, heterocycloalkynonyl, heterocycloalkenynonyl, or heteroaryl, (hereinafter sometimes referred to as “the cycle”).

As noted above, L ends with a —N(R¹¹)— group to which R₁₀ is attached. This means that the L chain atom closest to —R¹⁰ is the nitrogen atom of this —N(R¹¹)— group.

In embodiments, L represents:
—(C(R¹²)₂—)ₙ—C(═O)—N(R¹¹)—, wherein n is 1 or 2, or
—(C(R¹²)₂—)ₙ—N(R¹¹)—, wherein n is 2 or 3,
   wherein the R¹¹ and one of the R¹² of these groups together with the two atoms to which they are attached and any atom between said two atoms form the heteroaryl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl.

In preferred embodiments, L represents:
—C*(R¹²)₂—C(═O)—N(R¹¹)—,
—C*(R¹²)₂—C(R¹²)₂—C(R¹²)₂—N(R¹¹)—, or
—C*(R¹²)₂—C(R¹²)₂—N(R¹¹)—,
   wherein one of the R¹¹ and one of the R¹² (preferably one of the attached to the carbon atom indicated with a *) together with the two atoms to which they are attached and any atom between said two atoms form the above noted cycle.

In more preferred embodiments, the R¹² of these groups that form the heteroaryl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl is attached to the carbon atom in L closest to G, J, X or Q, i.e. the carbon atoms indicated by a star above.

In embodiments, the R¹¹ and the R¹² form a heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl, preferably a heterocycloalkyl or a heterocycloalkanonyl. In preferred embodiments, the R¹¹ and the R¹² form pyrrolidinyl (preferably pyrrolidin-3-yl), succinimidyl, (preferably succinimid-3-yl), or piperidinyl (preferably piperidin-4-yl).

In more preferred embodiments, the R¹¹ and the R¹² form pyrrolidinyl (preferably pyrrolidin-3-yl).

In alternative more preferred embodiments, the R¹¹ and the R¹² form succinimidyl (preferably succinimid-3-yl).

In alternative more preferred embodiments, the R¹¹ and the R¹² form piperidinyl (preferably piperidin-4-yl).

In embodiments, any other R¹¹ represents H.

In embodiments, any other R¹² represents H.

In embodiments, R¹⁰ represents H or —C(═O)—R¹⁵, preferably —C(═O)—R¹⁵. In preferred embodiments, R¹⁰ represents H or —C(═O)—R²⁰, preferably —C(═O)—R²⁰.

In embodiments wherein the R¹¹ and the R¹² form a heterocycloalkyl (preferably pyrrolidinyl or piperidinyl), R¹⁰ represents H or —C(═O)—R¹⁵ (preferably —C(═O)—R²⁰). In preferred such embodiments R¹⁰ represents H. In alternative more preferred embodiments R¹⁰ represents —C(═O)—R¹⁵ (preferably —C(═O)—R²⁰).

In embodiments wherein the R¹¹ and the R¹² form a heterocycloalkanonyl (preferably succinimidyl), R¹⁰ represents H or —C(═O)—R¹⁵ (preferably —C(═O)—R²⁰). In preferred such embodiments R¹⁰ represents H.

In embodiments, R¹⁵ (or R²⁰ as the case may be) represents one of the following groups:
alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, aryl, or heteroaryl,
preferably cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, aryl, or heteroaryl, each of which preferably being 5- or 6-membered,
more preferably cycloalkyl, cycloalkenyl, cycloalkynyl, cycloalkenynyl, heterocycloalkyl, heterocycloalkenyl, heterocycloalkynyl, heterocycloalkenynyl, or aryl, each of which preferably being 5- or 6-membered,
yet more preferably cycloalkyl, heterocycloalkyl, or aryl, each of which preferably being 5- or 6-membered,
   each of which independently unsubstituted or substituted with one or more R¹⁶. Preferred such cycloalkyls include cyclohexyl. Preferred such heterocycloalkyls include piperidinyl (preferably piperidin-4-yl or piperidin-3-yl) and pyrrolidinyl (preferably pyrrolidin-3-yl). Preferred such aryls include phenyl.

In embodiments, the group in R¹⁵ is unsubstituted.

In embodiments, the group in R¹⁵ is heterocycloalkyl, heterocycloalkenyl, heterocycloalkynyl, heterocycloalkenynyl, or heteroaryl, R¹⁶ is preferably substituting a heteroatom of these groups (preferably N).

In embodiments, R¹⁶ is:
a halogen atom, alkylcarbonyl, alkenylcarbonyl, alkynylcarbonyl, alkenynylcarbonyl, alkoxycarbonyl, alkylcarbamoyl, alkenylcarbamoyl, alkynylcarbamoyl, alkenynylcarbamoyl, or hydroxyl,
preferably a halogen atom, alkylcarbonyl, alkoxycarbonyl, alkylcarbamoyl, or hydroxyl,
more preferably a halogen atom, alkylcarbonyl, alkylcarbamoyl, or hydroxyl.
   Preferred such halogen atoms include F. Preferred such alkylcarbonyls include —C(═O)—CH₃. Preferred such alkoxycarbonyls include —C(═O)—O-t-butyl. Preferred such alkylcarbamoyls include —NH—C(═O)—CH₃.

When R¹⁶ is substituting a nitrogen atom, R¹⁶ is preferably alkylcarbonyl or alkoxycarbonyl, preferably alkylcarbonyl.

Non-limiting examples of compounds of embodiments D) that may or may not fall within one or more formula (II), (III), and/or (IV) below, include:

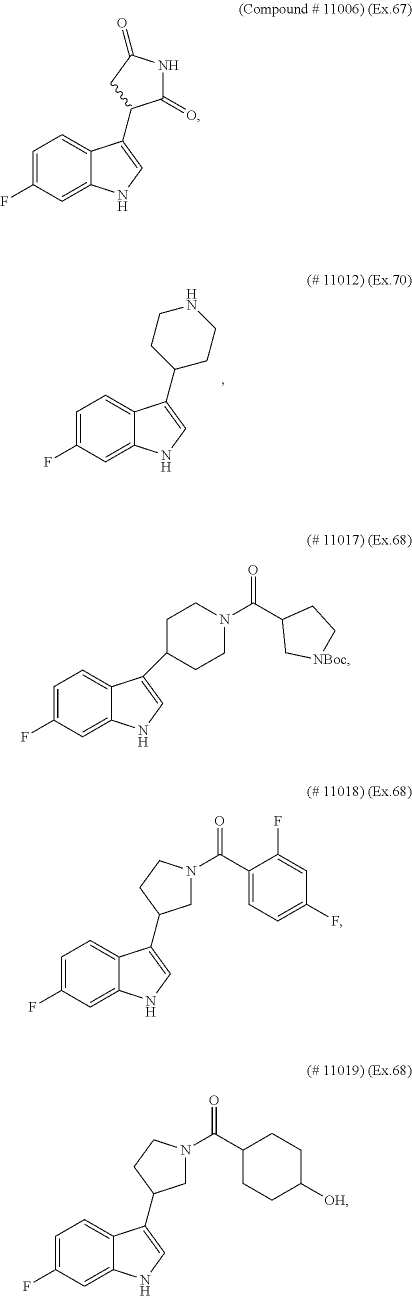 [SMILES: O=C1CC(c2c[nH]c3cc(F)ccc23)C(=O)N1 ; Fc1ccc2c(C3CCNCC3)c[nH]c2c1 ; CC(C)(C)OC(=O)N1CCC(C(=O)N2CCC(c3c[nH]c4cc(F)ccc34)CC2)C1 ; O=C(c1ccc(F)cc1F)N1CCC(c2c[nH]c3cc(F)ccc23)C1 ; O=C(C1CCC(O)CC1)N1CCC(c2c[nH]c3cc(F)ccc23)C1]

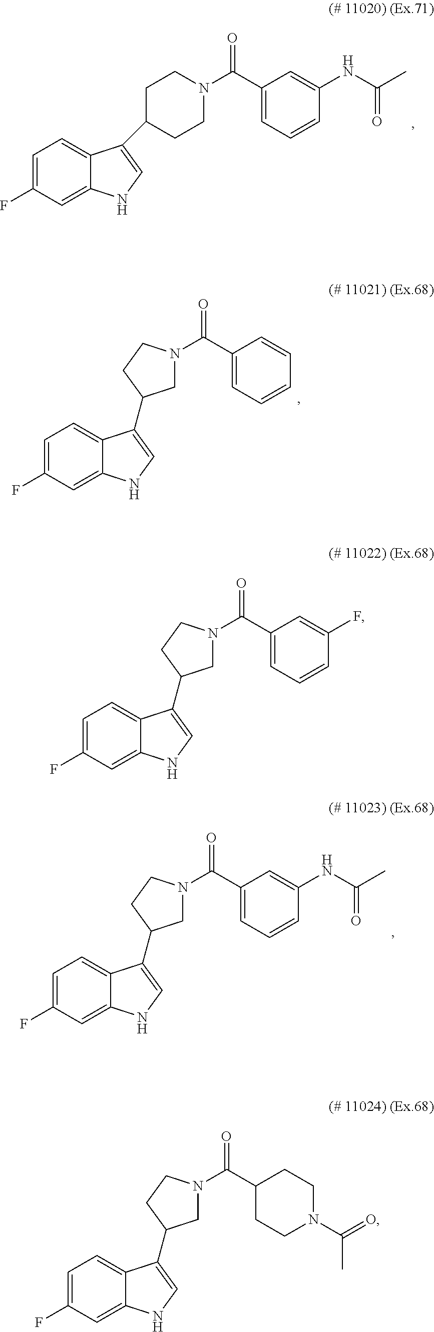 [SMILES: CC(=O)Nc1cccc(C(=O)N2CCC(c3c[nH]c4cc(F)ccc34)CC2)c1 ; O=C(c1ccccc1)N1CCC(c2c[nH]c3cc(F)ccc23)C1 ; O=C(c1cccc(F)c1)N1CCC(c2c[nH]c3cc(F)ccc23)C1 ; CC(=O)Nc1cccc(C(=O)N2CCC(c3c[nH]c4cc(F)ccc34)C2)c1 ; CC(=O)N1CCC(C(=O)N2CCC(c3c[nH]c4cc(F)ccc34)C2)CC1]

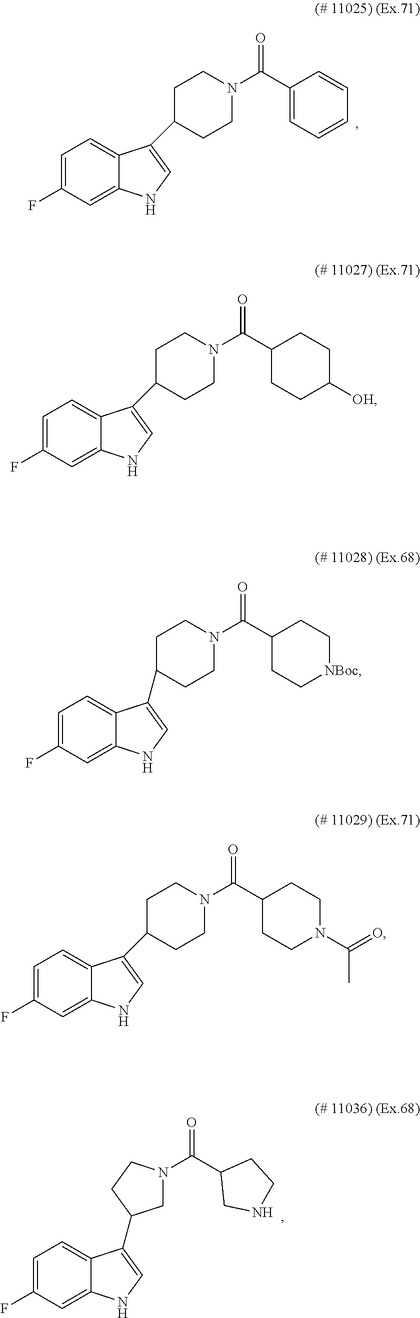 [SMILES: O=C(c1ccccc1)N1CCC(c2c[nH]c3cc(F)ccc23)CC1 ; O=C(C1CCC(O)CC1)N1CCC(c2c[nH]c3cc(F)ccc23)CC1 ; CC(C)(C)OC(=O)N1CCC(C(=O)N2CCC(c3c[nH]c4cc(F)ccc34)CC2)CC1 ; CC(=O)N1CCC(C(=O)N2CCC(c3c[nH]c4cc(F)ccc34)CC2)CC1 ; O=C(C1CCNC1)N1CCC(c2c[nH]c3cc(F)ccc23)C1]

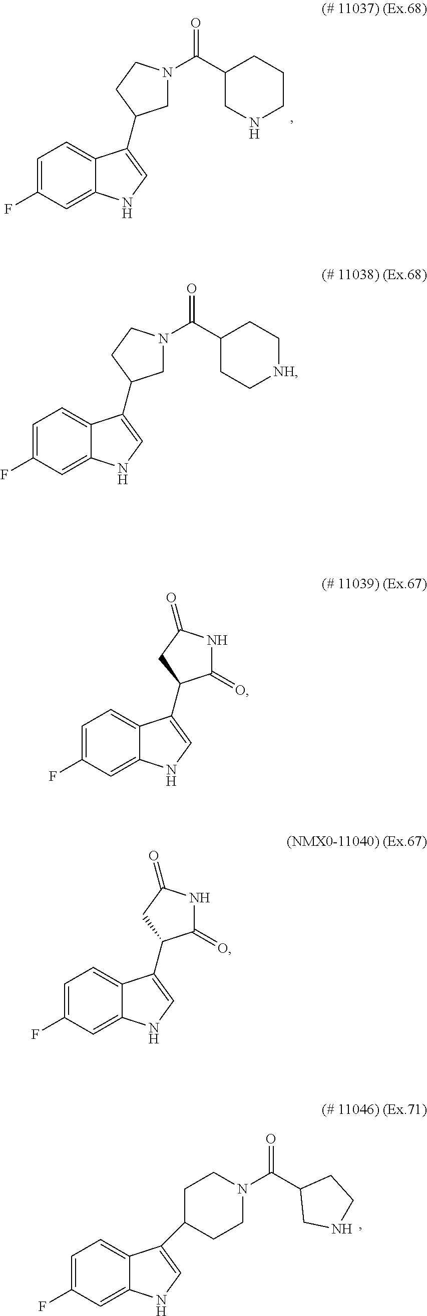 [SMILES: O=C(C1CCCNC1)N1CCC(c2c[nH]c3cc(F)ccc23)C1 ; O=C(C1CCNCC1)N1CCC(c2c[nH]c3cc(F)ccc23)C1 ; O=C1C[C@H](c2c[nH]c3cc(F)ccc23)C(=O)N1 ; O=C1C[C@@H](c2c[nH]c3cc(F)ccc23)C(=O)N1 ; O=C(C1CCNC1)N1CCC(c2c[nH]c3cc(F)ccc23)CC1]

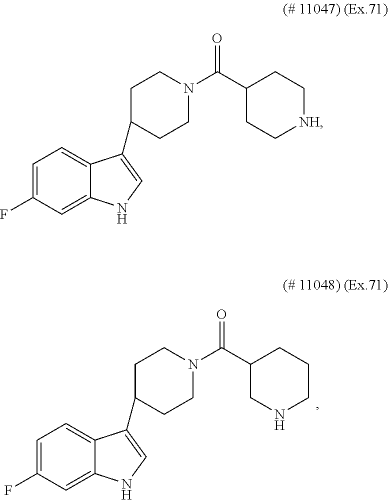 [SMILES: O=C(C1CCNCC1)N1CCC(c2c[nH]c3cc(F)ccc23)CC1 ; O=C(C1CCCNC1)N1CCC(c2c[nH]c3cc(F)ccc23)CC1]

or a pharmaceutically acceptable salt, ester, solvate, isomer, or tautomer thereof.

More preferred compounds include Compound #11006 and pharmaceutically acceptable salts, esters, solvates, isomers, or tautomers thereof.

### Compounds of Formula (II)

In embodiments, the compound of formula (I) above (including all of its embodiments and preferred embodiments) is, more specifically, of formula (II):

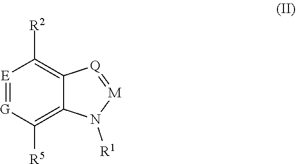

wherein R¹, R², R⁵, E, G, M and Q are as described above.

In preferred embodiments:
E represents —CH═,
G represents —C(R⁴)═, preferably wherein R⁴ represents a halogen atom, more preferably F,
M represents —N═ or —C(R⁷)═,
Q represents —C(R⁸)═, preferably —C(L-R¹⁰)═, wherein L and R¹⁰ are as defined above,
R¹ represents H,
R² represents H, and/or
R⁴ represents H, and
   with the proviso that the compound of formula (II) comprises exactly one -L-R¹⁰ group,
   or a pharmaceutically acceptable salt, ester, solvate, isomer, or tautomer thereof.

In preferred embodiments M represents —C(R⁷)═, preferably —CH═.

In alternative embodiments, M represents —N═.

### Compounds of Formula (III)

In embodiments, the compounds of formulas (I) and (II) above (including all of their embodiments and preferred embodiments) are, more specifically, fluorinated compounds of formula (III):

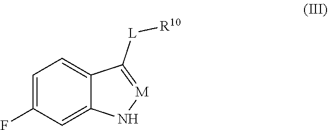

wherein L, M, and R¹⁰ are as defined above,
or a pharmaceutically acceptable salt, ester, solvate, isomer, or tautomer thereof.

In preferred embodiments M represents —C(R⁷)═, preferably —CH═.

In alternative embodiments, M represents —N═.

### Compounds of Formula (IV)

In embodiments, the compounds of formulas (I), (II), and (Ill) above (including all of their embodiments and preferred embodiments) are, more specifically, fluoroindole compounds of formula (IV):

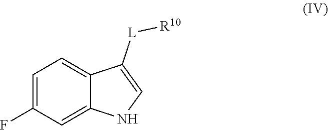

wherein L and R¹⁰ are as defined above,
or a pharmaceutically acceptable salt, ester, solvate, isomer, or tautomer thereof.

### Specific Compounds

In embodiments, the compound is:

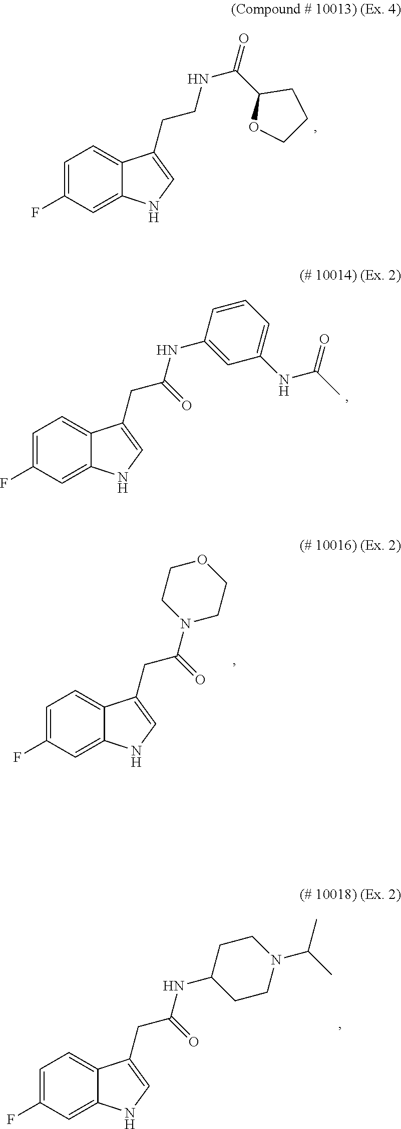 [SMILES: O=C(NCCc1c[nH]c2cc(F)ccc12)[C@H]1CCCO1 ; CC(=O)Nc1cccc(NC(=O)Cc2c[nH]c3cc(F)ccc23)c1 ; O=C(Cc1c[nH]c2cc(F)ccc12)N1CCOCC1 ; CC(C)N1CCC(NC(=O)Cc2c[nH]c3cc(F)ccc23)CC1]

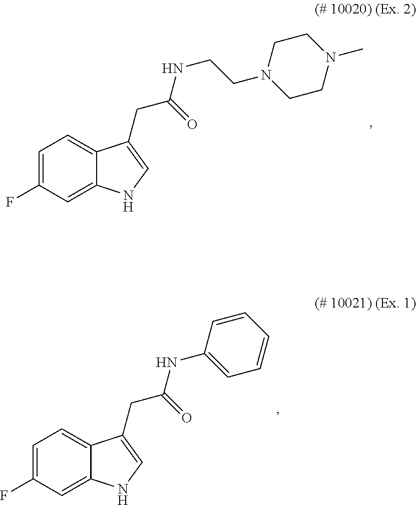 [SMILES: CN1CCN(CCNC(=O)Cc2c[nH]c3cc(F)ccc23)CC1 ; O=C(Cc1c[nH]c2cc(F)ccc12)Nc1ccccc1]

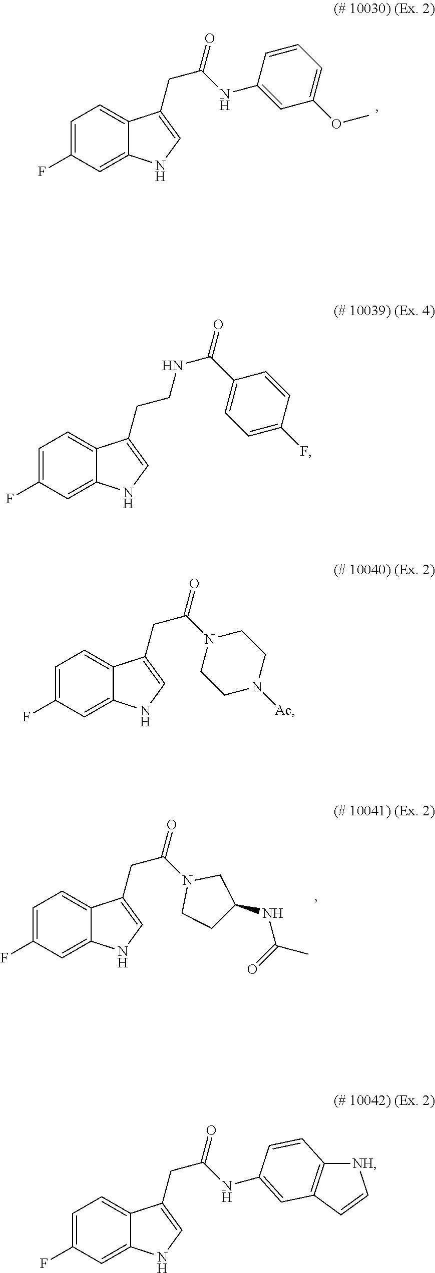 [SMILES: COc1cccc(NC(=O)Cc2c[nH]c3cc(F)ccc23)c1 ; O=C(NCCc1c[nH]c2cc(F)ccc12)c1ccc(F)cc1 ; CC(=O)N1CCN(C(=O)Cc2c[nH]c3cc(F)ccc23)CC1 ; CC(=O)N[C@H]1CCN(C(=O)Cc2c[nH]c3cc(F)ccc23)C1 ; O=C(Cc1c[nH]c2cc(F)ccc12)Nc1ccc2[nH]ccc2c1]

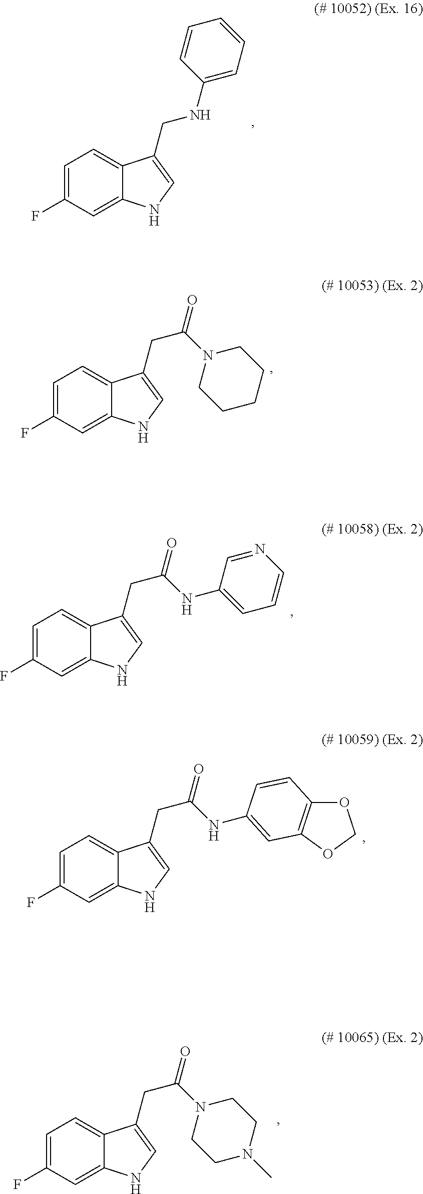 [SMILES: Fc1ccc2c(CNc3ccccc3)c[nH]c2c1 ; O=C(Cc1c[nH]c2cc(F)ccc12)N1CCCCC1 ; O=C(Cc1c[nH]c2cc(F)ccc12)Nc1cccnc1 ; O=C(Cc1c[nH]c2cc(F)ccc12)Nc1ccc2c(c1)OCO2 ; CN1CCN(C(=O)Cc2c[nH]c3cc(F)ccc23)CC1]

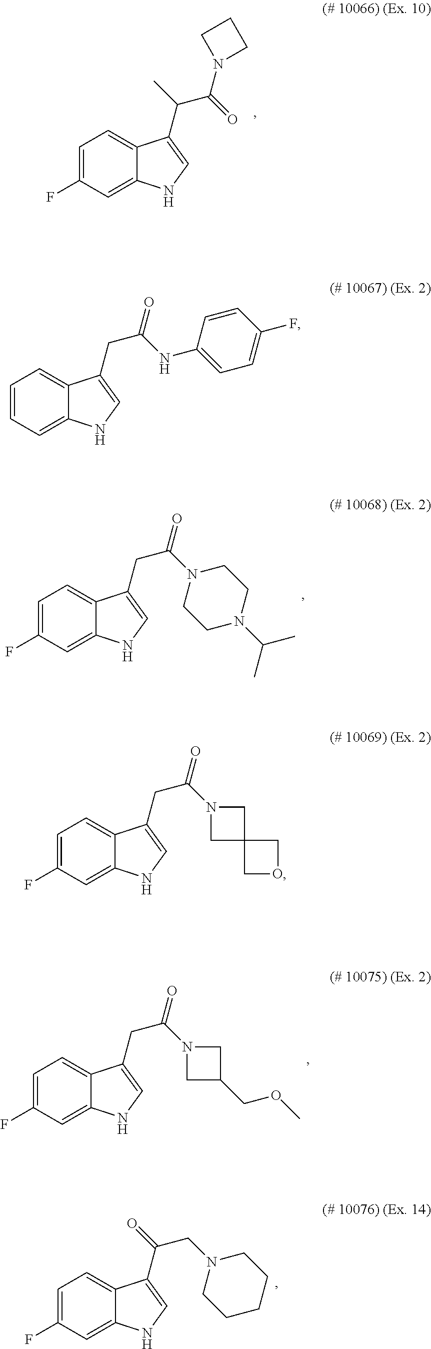 [SMILES: CC(C(=O)N1CCC1)c1c[nH]c2cc(F)ccc12 ; O=C(Cc1c[nH]c2ccccc12)Nc1ccc(F)cc1 ; CC(C)N1CCN(C(=O)Cc2c[nH]c3cc(F)ccc23)CC1 ; O=C(Cc1c[nH]c2cc(F)ccc12)N1CC2(COC2)C1 ; COCC1CN(C(=O)Cc2c[nH]c3cc(F)ccc23)C1 ; O=C(CN1CCCCC1)c1c[nH]c2cc(F)ccc12]

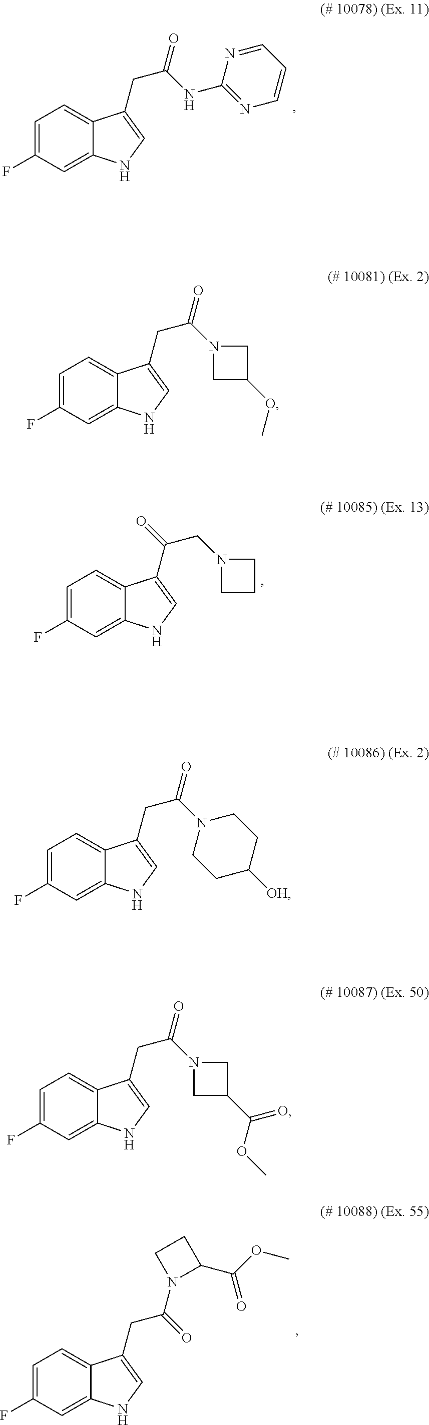 [SMILES: O=C(Cc1c[nH]c2cc(F)ccc12)Nc1ncccn1 ; COC1CN(C(=O)Cc2c[nH]c3cc(F)ccc23)C1 ; O=C(CN1CCC1)c1c[nH]c2cc(F)ccc12 ; O=C(Cc1c[nH]c2cc(F)ccc12)N1CCC(O)CC1 ; COC(=O)C1CN(C(=O)Cc2c[nH]c3cc(F)ccc23)C1 ; COC(=O)C1CCN1C(=O)Cc1c[nH]c2cc(F)ccc12]

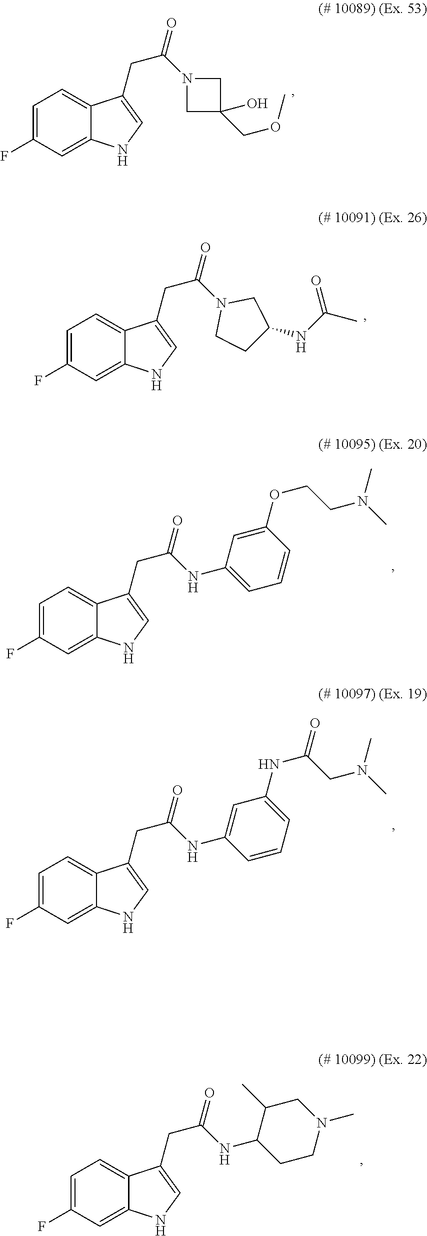 [SMILES: COCC1(O)CN(C(=O)Cc2c[nH]c3cc(F)ccc23)C1 ; CC(=O)N[C@@H]1CCN(C(=O)Cc2c[nH]c3cc(F)ccc23)C1 ; CN(C)CCOc1cccc(NC(=O)Cc2c[nH]c3cc(F)ccc23)c1 ; CN(C)CC(=O)Nc1cccc(NC(=O)Cc2c[nH]c3cc(F)ccc23)c1 ; CC1CN(C)CCC1NC(=O)Cc1c[nH]c2cc(F)ccc12]

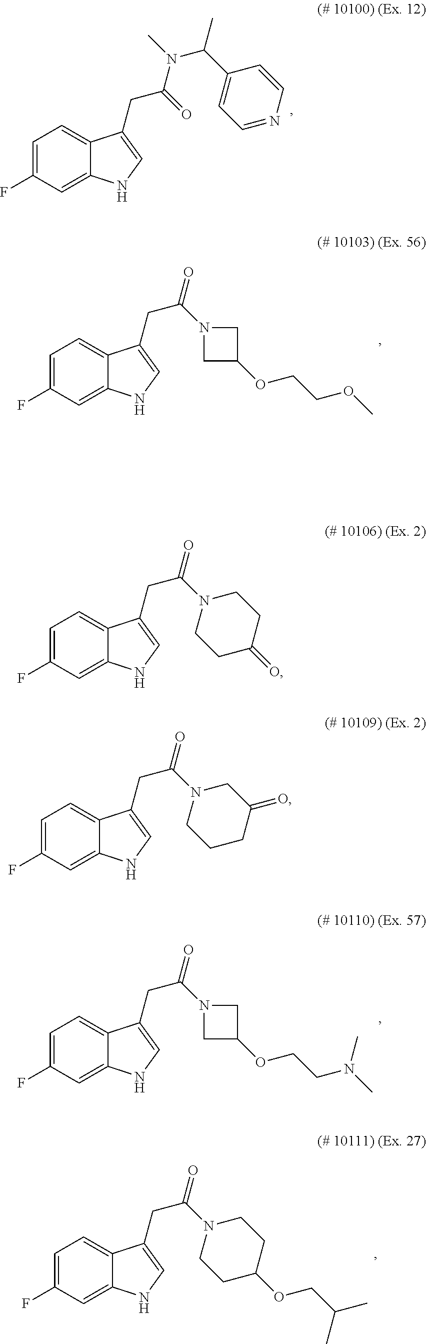 [SMILES: CC(c1ccncc1)N(C)C(=O)Cc1c[nH]c2cc(F)ccc12 ; COCCOC1CN(C(=O)Cc2c[nH]c3cc(F)ccc23)C1 ; O=C1CCN(C(=O)Cc2c[nH]c3cc(F)ccc23)CC1 ; O=C1CCCN(C(=O)Cc2c[nH]c3cc(F)ccc23)C1 ; CN(C)CCOC1CN(C(=O)Cc2c[nH]c3cc(F)ccc23)C1 ; CC(C)COC1CCN(C(=O)Cc2c[nH]c3cc(F)ccc23)CC1]

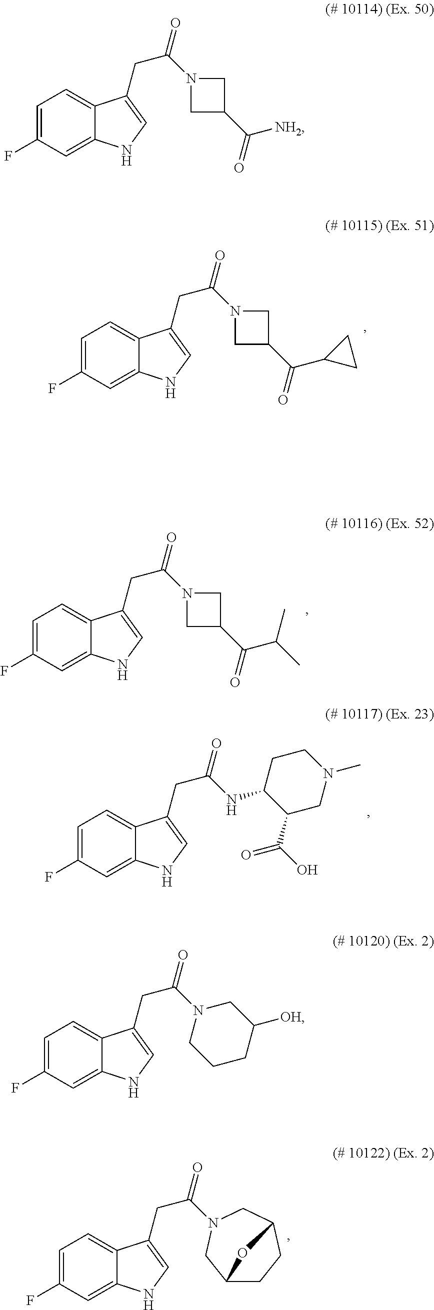 [SMILES: NC(=O)C1CN(C(=O)Cc2c[nH]c3cc(F)ccc23)C1 ; O=C(C1CC1)C1CN(C(=O)Cc2c[nH]c3cc(F)ccc23)C1 ; CC(C)C(=O)C1CN(C(=O)Cc2c[nH]c3cc(F)ccc23)C1 ; CN1CC[C@@H](NC(=O)Cc2c[nH]c3cc(F)ccc23)[C@@H](C(=O)O)C1 ; O=C(Cc1c[nH]c2cc(F)ccc12)N1CCCC(O)C1 ; O=C(Cc1c[nH]c2cc(F)ccc12)N1C[C@H]2CC[C@@H](C1)O2]

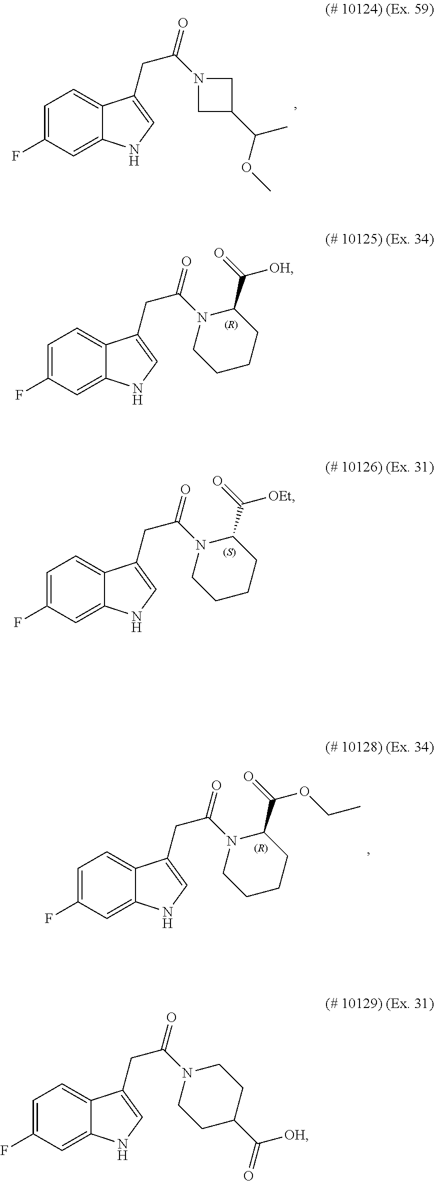

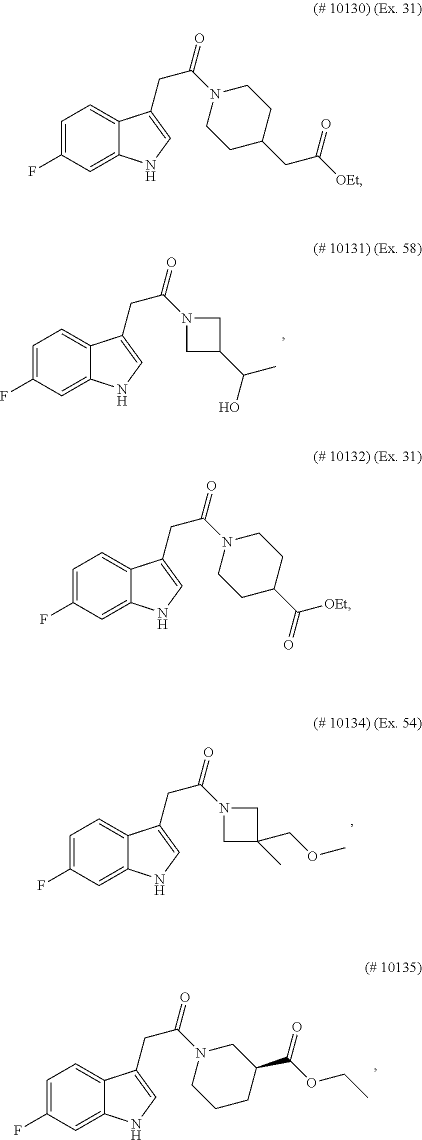 [SMILES: CCOC(=O)CC1CCN(C(=O)Cc2c[nH]c3cc(F)ccc23)CC1 ; CC(O)C1CN(C(=O)Cc2c[nH]c3cc(F)ccc23)C1 ; CCOC(=O)C1CCN(C(=O)Cc2c[nH]c3cc(F)ccc23)CC1 ; COCC1(C)CN(C(=O)Cc2c[nH]c3cc(F)ccc23)C1 ; CCOC(=O)[C@H]1CCCN(C(=O)Cc2c[nH]c3cc(F)ccc23)C1]

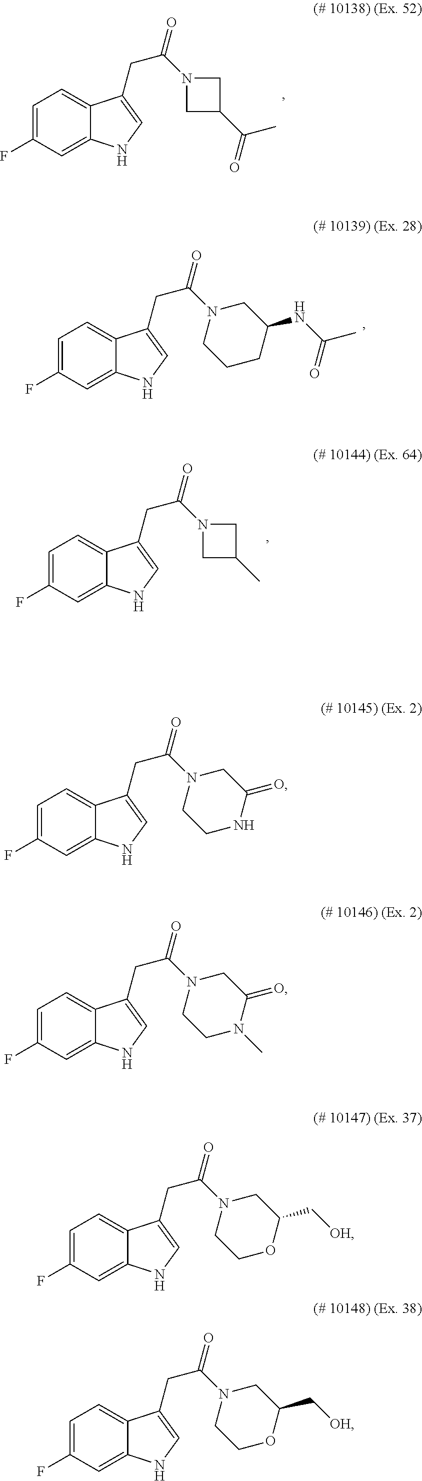

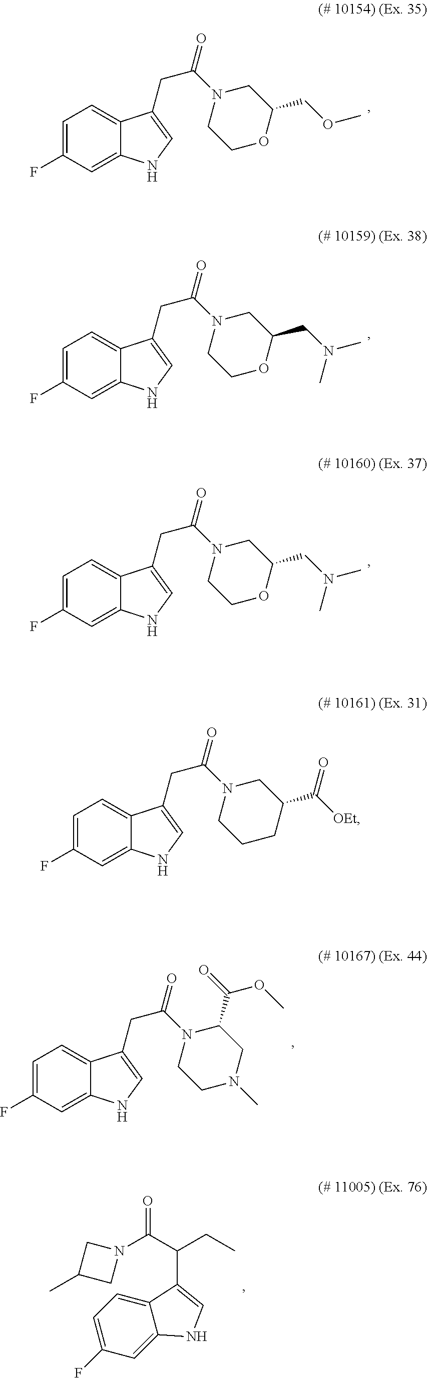

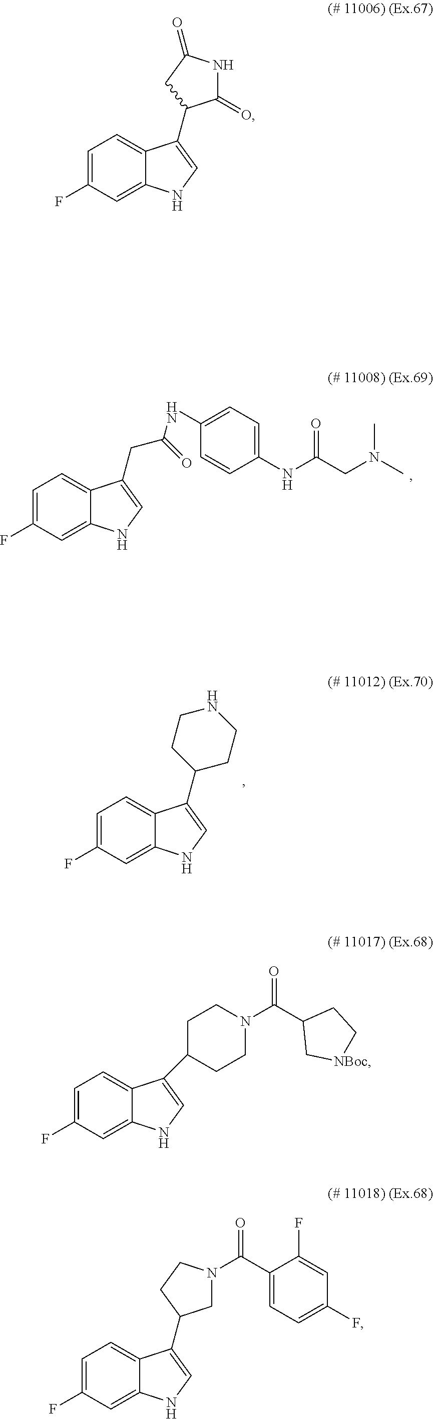 [SMILES: O=C1CC(c2c[nH]c3cc(F)ccc23)C(=O)N1 ; CN(C)CC(=O)Nc1ccc(NC(=O)Cc2c[nH]c3cc(F)ccc23)cc1 ; Fc1ccc2c(C3CCNCC3)c[nH]c2c1 ; CC(C)(C)OC(=O)N1CCC(C(=O)N2CCC(c3c[nH]c4cc(F)ccc34)CC2)C1 ; O=C(c1ccc(F)cc1F)N1CCC(c2c[nH]c3cc(F)ccc23)C1]

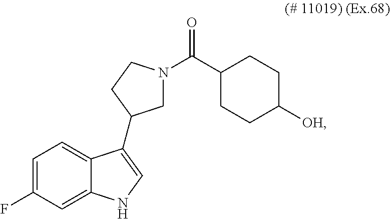 [SMILES: O=C(C1CCC(O)CC1)N1CCC(c2c[nH]c3cc(F)ccc23)C1]

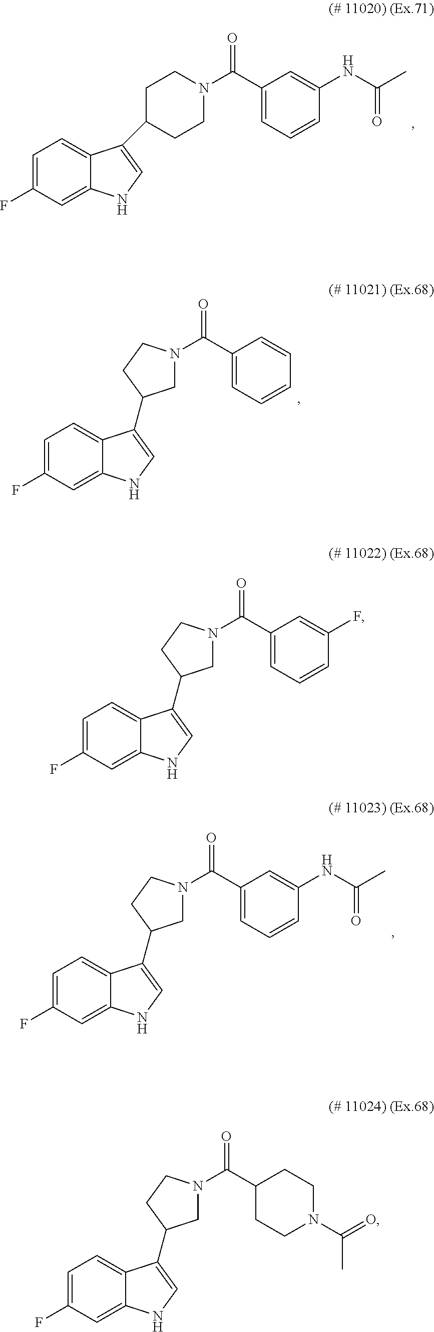 [SMILES: CC(=O)Nc1cccc(C(=O)N2CCC(c3c[nH]c4cc(F)ccc34)CC2)c1 ; O=C(c1ccccc1)N1CCC(c2c[nH]c3cc(F)ccc23)C1 ; O=C(c1cccc(F)c1)N1CCC(c2c[nH]c3cc(F)ccc23)C1 ; CC(=O)Nc1cccc(C(=O)N2CCC(c3c[nH]c4cc(F)ccc34)C2)c1 ; CC(=O)N1CCC(C(=O)N2CCC(c3c[nH]c4cc(F)ccc34)C2)CC1]

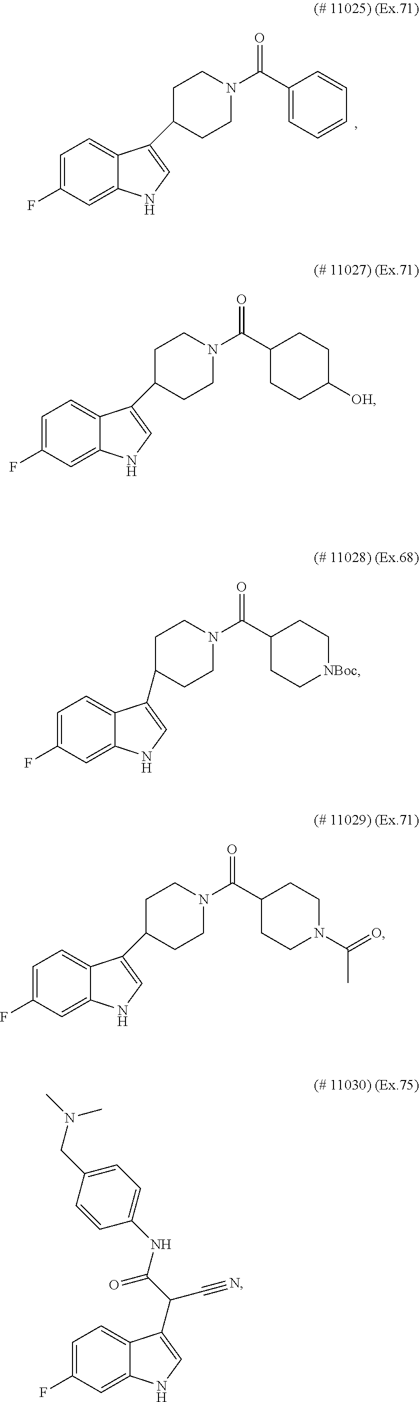 [SMILES: O=C(c1ccccc1)N1CCC(c2c[nH]c3cc(F)ccc23)CC1 ; O=C(C1CCC(O)CC1)N1CCC(c2c[nH]c3cc(F)ccc23)CC1 ; CC(C)(C)OC(=O)N1CCC(C(=O)N2CCC(c3c[nH]c4cc(F)ccc34)CC2)CC1 ; CC(=O)N1CCC(C(=O)N2CCC(c3c[nH]c4cc(F)ccc34)CC2)CC1 ; CN(C)Cc1ccc(NC(=O)C(C#N)c2c[nH]c3cc(F)ccc23)cc1]

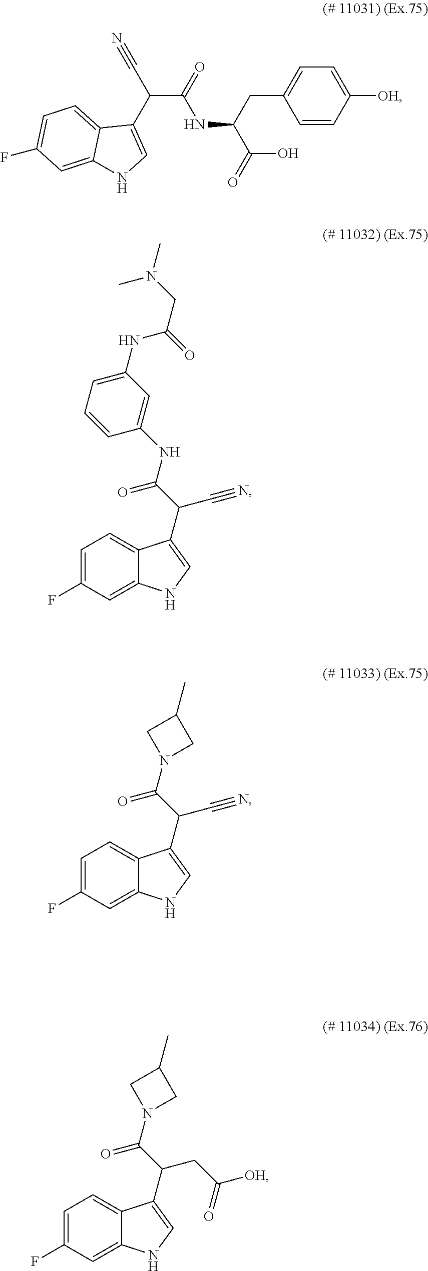 [SMILES: N#CC(C(=O)N[C@@H](Cc1ccc(O)cc1)C(=O)O)c1c[nH]c2cc(F)ccc12 ; CN(C)CC(=O)Nc1cccc(NC(=O)C(C#N)c2c[nH]c3cc(F)ccc23)c1 ; CC1CN(C(=O)C(C#N)c2c[nH]c3cc(F)ccc23)C1 ; CC1CN(C(=O)C(CC(=O)O)c2c[nH]c3cc(F)ccc23)C1]

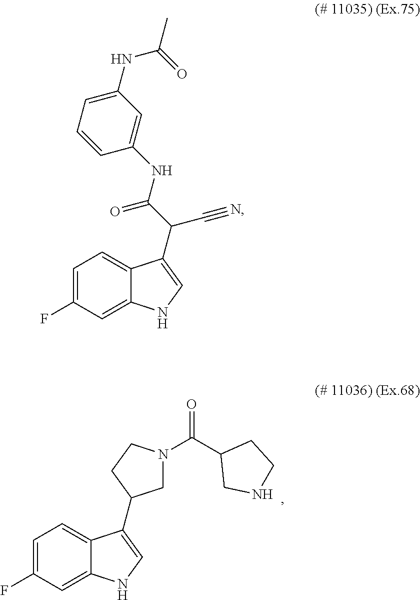 [SMILES: CC(=O)Nc1cccc(NC(=O)C(C#N)c2c[nH]c3cc(F)ccc23)c1 ; O=C(C1CCNC1)N1CCC(c2c[nH]c3cc(F)ccc23)C1]

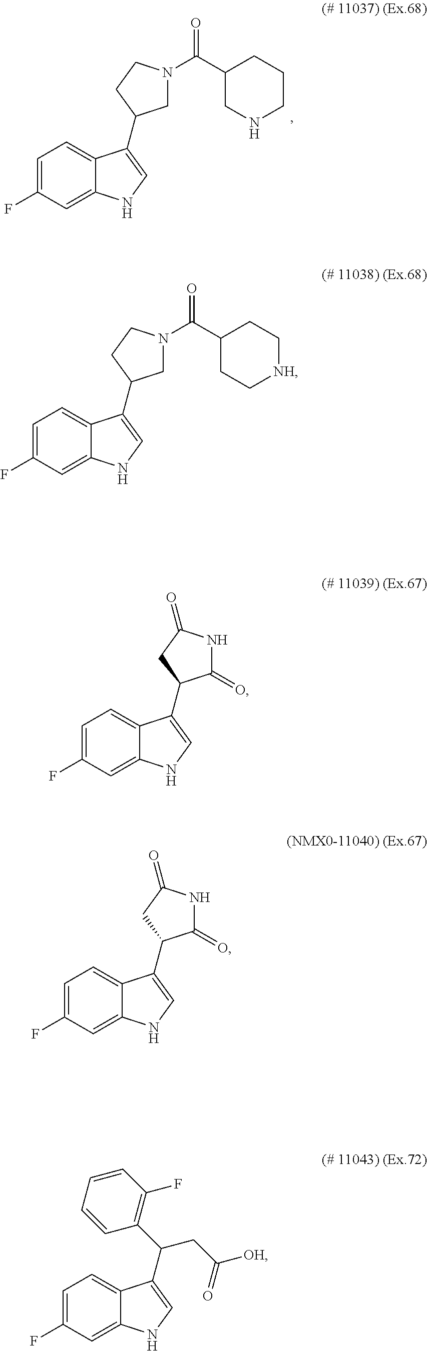 [SMILES: O=C(C1CCCNC1)N1CCC(c2c[nH]c3cc(F)ccc23)C1 ; O=C(C1CCNCC1)N1CCC(c2c[nH]c3cc(F)ccc23)C1 ; O=C1C[C@H](c2c[nH]c3cc(F)ccc23)C(=O)N1 ; O=C1C[C@@H](c2c[nH]c3cc(F)ccc23)C(=O)N1 ; O=C(O)CC(c1ccccc1F)c1c[nH]c2cc(F)ccc12]

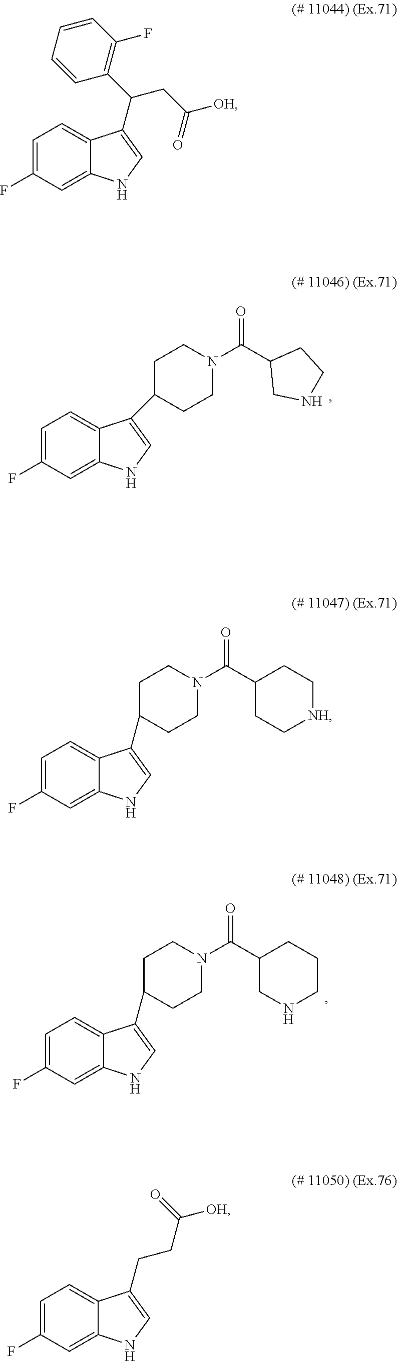 [SMILES: O=C(O)CC(c1ccccc1F)c1c[nH]c2cc(F)ccc12 ; O=C(C1CCNC1)N1CCC(c2c[nH]c3cc(F)ccc23)CC1 ; O=C(C1CCNCC1)N1CCC(c2c[nH]c3cc(F)ccc23)CC1 ; O=C(C1CCCNC1)N1CCC(c2c[nH]c3cc(F)ccc23)CC1 ; O=C(O)CCc1c[nH]c2cc(F)ccc12]

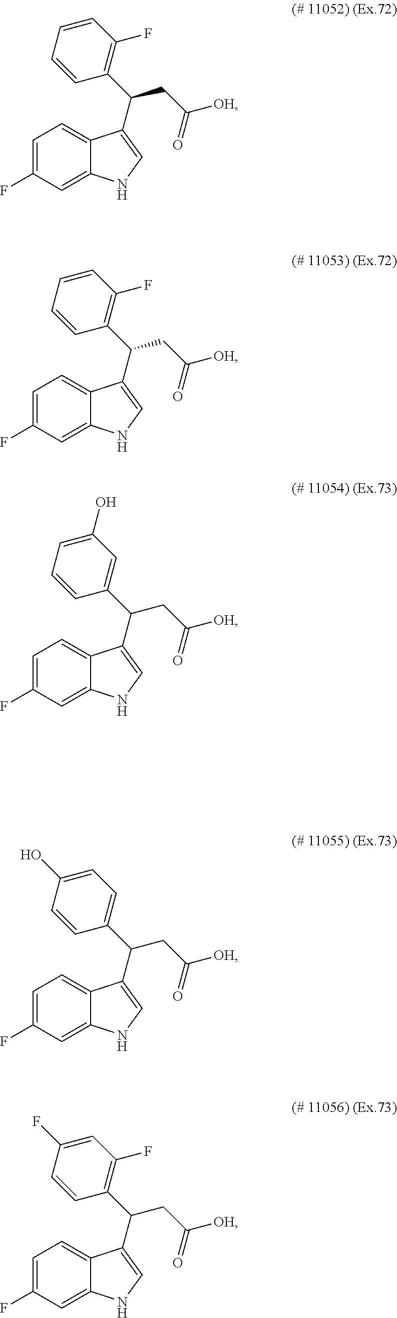 [SMILES: O=C(O)C[C@H](c1ccccc1F)c1c[nH]c2cc(F)ccc12 ; O=C(O)C[C@@H](c1ccccc1F)c1c[nH]c2cc(F)ccc12 ; O=C(O)CC(c1cccc(O)c1)c1c[nH]c2cc(F)ccc12 ; O=C(O)CC(c1ccc(O)cc1)c1c[nH]c2cc(F)ccc12 ; O=C(O)CC(c1ccc(F)cc1F)c1c[nH]c2cc(F)ccc12]

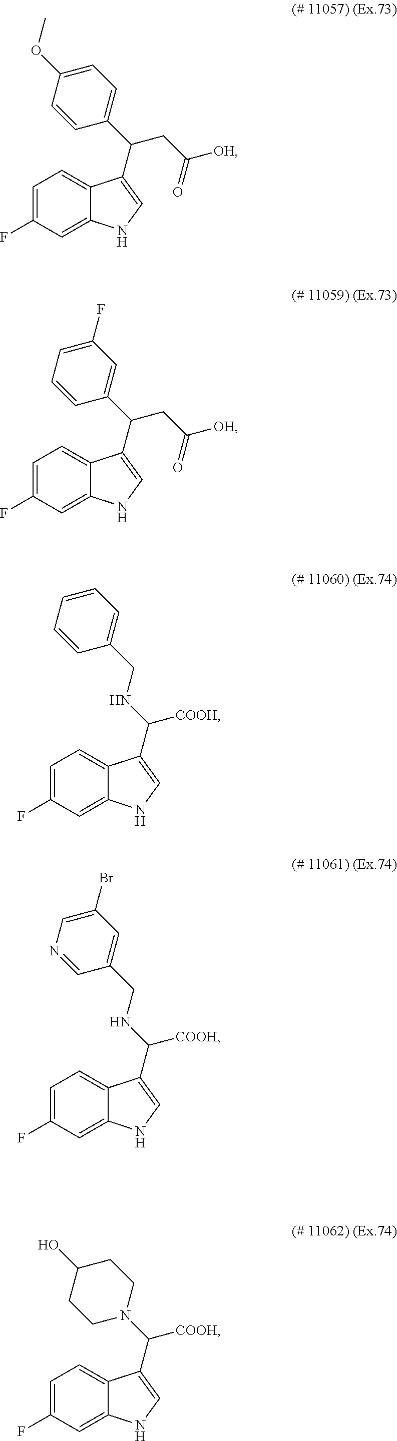 [SMILES: COc1ccc(C(CC(=O)O)c2c[nH]c3cc(F)ccc23)cc1 ; O=C(O)CC(c1cccc(F)c1)c1c[nH]c2cc(F)ccc12 ; O=C(O)C(NCc1ccccc1)c1c[nH]c2cc(F)ccc12 ; O=C(O)C(NCc1cncc(Br)c1)c1c[nH]c2cc(F)ccc12 ; O=C(O)C(c1c[nH]c2cc(F)ccc12)N1CCC(O)CC1]

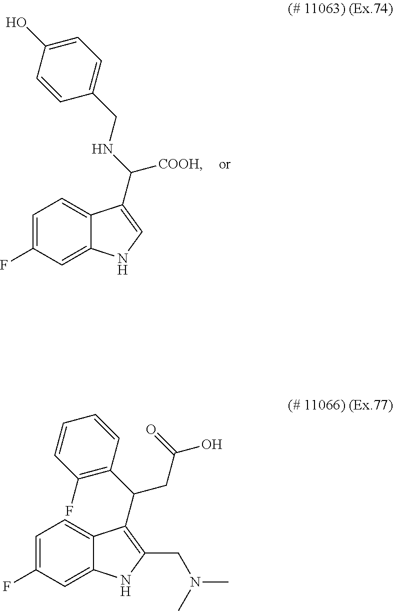 [SMILES: O=C(O)C(NCc1ccc(O)cc1)c1c[nH]c2cc(F)ccc12 ; CN(C)Cc1[nH]c2cc(F)ccc2c1C(CC(=O)O)c1ccccc1F]

or a pharmaceutically acceptable salt, ester, solvate, isomer, or tautomer thereof.

Preferred compounds include Compounds #10014, 10018, 10035, 10040, 10053, 10066, 10076, 10086, 10095, 10097, 10159, 11005, 11006, 11008, 11043, 11052 (enantiomer of 11043), 11053 (enantiomer of 11043), 11054, 11055, 11066, and 11073, and pharmaceutically acceptable salts, esters, solvates, isomers, or tautomers thereof.

More preferred compounds include Compounds #10018, 10040, 10086, 10095, 10097, 11005, 11006, 11008, 11032, 11053, 11066, and 11073, and pharmaceutically acceptable salts, esters, solvates, isomers, or tautomers thereof.

Even more preferred compounds include Compounds #10095, 10097, 11005, 11006, 11008, 11053, and pharmaceutically acceptable salts, esters, solvates, isomers, or tautomers thereof.

Most preferred compounds with HRAS activity include 11095, 11097, and 11032, and pharmaceutically acceptable salts, esters, solvates, isomers, or tautomers thereof.

Most preferred compounds with KRAS activity include 10018, 10040, 10086, and 11066, and pharmaceutically acceptable salts, esters, solvates, isomers, or tautomers thereof.

### Salts

The present invention relates to the compounds of the invention as hereinbefore defined as well as to salts thereof. The term “salt(s)”, as employed herein, denotes acidic salts formed with inorganic and/or organic acids, as well as basic salts formed with inorganic and/or organic bases. Salts for use in pharmaceutical compositions will be pharmaceutically acceptable salts, but other salts may be useful in the production of the compounds of the invention.

The term “pharmaceutically acceptable salt” refers to salts of compounds of the present invention that are pharmacologically acceptable and substantially non-toxic to the subject to which they are administered. More specifically, these salts retain the biological effectiveness and properties of the compounds of the invention and are formed from suitable non-toxic organic or inorganic acids or bases.

For example, these salts include acid addition salts of the compounds of the invention which are sufficiently basic to form such salts. Such acid addition salts include acetates, adipates, alginates, lower alkanesulfonates such as a methanesulfonates, trifluoromethanesulfonatse or ethanesulfonates, arylsulfonates such as a benzenesulfonates, 2-naphthalenesulfonates, or toluenesulfonates (also known as tosylates), ascorbates, aspartates, benzoates, benzenesulfonates, bisulfates, borates, butyrates, citrates, camphorates, camphorsulfonates, cinnamates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydrogen sulphates, 2-hydroxyethanesulfonates, itaconates, lactates, maleates, mandelates, methanesulfonates, nicotinates, nitrates, oxalates, pamoates, pectinates, perchlorates, persulfates, 3-phenylpropionates, phosphates, picrates, pivalates, propionates, salicylates, succinates, sulfates, sulfonates, tartrates, thiocyanates, undecanoates and the like.

Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

Also, where the compounds of the invention are sufficiently acidic, the salts of the invention include base salts formed with an inorganic or organic base. Such salts include alkali metal salts such as sodium, lithium, and potassium salts; alkaline earth metal salts such as calcium and magnesium salts; metal salts such as aluminium salts, iron salts, zinc salts, copper salts, nickel salts and a cobalt salts; inorganic amine salts such as ammonium or substituted ammonium salts, such as trimethylammonium salts; and salts with organic bases (for example, organic amines) such as chloroprocaine salts, dibenzylamine salts, dicyclohexylamine salts, dicyclohexylamines, diethanolamine salts, ethylamine salts (including diethylamine salts and triethylamine salts), ethylenediamine salts, glucosamine salts, guanidine salts, methylamine salts (including dimethylamine salts and trimethylamine salts), morpholine salts, morpholine salts, N,N′-dibenzylethylenediamine salts, N-benzyl-phenethylamine salts, N-methylglucamine salts, phenylglycine alkyl ester salts, piperazine salts, piperidine salts, procaine salts, t-butyl amines salts, tetramethylammonium salts, t-octylamine salts, tris-(2-hydroxyethyl)amine salts, and tris(hydroxymethyl)aminomethane salts.

Such salts can be formed quite readily by those skilled in the art using standard techniques. Indeed, the chemical modification of a pharmaceutical compound (i.e., drug) into a salt is a technique well known to pharmaceutical chemists, (See, e.g., H. Ansel et. al., Pharmaceutical Dosage Forms and Drug Delivery Systems (6th Ed. 1995) at pp. 196 and 1456-1457). Salts of the compounds of the invention may be formed, for example, by reacting a compound of the invention with an amount of acid or base, such as an equivalent amount, in a medium such as one in which the salt precipitates or in an aqueous medium followed by lyophilization.

### Esters

The present invention relates to the compounds of the invention as hereinbefore defined as well as to the esters thereof. The term “ester(s)”, as employed herein, refers to compounds of the invention or salts thereof in which at least on hydroxy group has been converted to a corresponding ester using, for example, inorganic or organic anhydrides, acids or acid chlorides. Esters for use in pharmaceutical compositions will be pharmaceutically acceptable esters, but other esters may be useful in the production of the compounds of the invention.

The term “pharmaceutically acceptable ester” refers to esters of compounds of the present invention that are pharmacologically acceptable and substantially non-toxic to the subject to which they are administered. More specifically, these esters retain the biological effectiveness and properties of the compounds of the invention and act as prodrugs which, when absorbed into the bloodstream of a warm-blooded animal, cleave in such a manner as to produce the parent alcohol.

Esters of the present compounds include among others the following groups
a) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, n-butyl, methyl, ethyl, propyl, isopropyl, butyl, isobutyl or pentyl), alkoxyalkyl (for example, methoxymethyl, acetoxymethyl and 2,2-dimethylpropionyloxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino);
b) sulfonate esters, such as alkyl- or aralkylsulfonyl (for example, methanesulfonyl);
c) amino acid esters (for example, L-valyl or L-isoleucyl);
d) phosphonate esters; and
e) mono-, di- or triphosphate esters (including phosphoramidic cyclic esters). The phosphate esters may be further esterified by, for example, a C₁₋₂₀ alcohol or reactive derivative thereof, or by a 2,3-di(C₆₋₂₄)acyl glycerol.

Further information concerning examples of and the use of esters for the delivery of pharmaceutical compounds is available in Design of Prodrugs. Bundgaard H ed. (Elsevier, 1985). See also, H. Ansel et. al., Pharmaceutical Dosage Forms and Drug Delivery Systems (6th Ed. 1995) at pp. 108-109; Krogsgaard-Larsen, et. al., Textbook of Drug Design and Development (2d Ed. 1996) at pp. 152-191.

The compounds of this invention may be esterified by a variety of conventional procedures including reacting the appropriate anhydride, carboxylic acid or acid chloride with the alcohol group of a compound of this invention. For example, an appropriate anhydride may be reacted with an alcohol in the presence of a base, such as 1,8-bis[dimethylamino]naphthalene or N,N-dimethylaminopyridine, to facilitate acylation. Also, an appropriate carboxylic acid can be reacted with the alcohol in the presence of a dehydrating agent such as dicyclohexylcarbodiimide, 1-[3-dimethylaminopropyl]-3-ethylcarbodiimide or other water soluble dehydrating agents which are used to drive the reaction by the removal of water, and, optionally, an acylation catalyst. Esterification can also be effected using the appropriate carboxylic acid in the presence of trifluoroacetic anhydride and, optionally, pyridine, or in the presence of N,N-carbonyldiimidazole with pyridine. Reaction of an acid chloride with the alcohol can be carried out with an acylation catalyst such as 4-DMAP or pyridine. When a compound of the invention contains a number of free hydroxy group, those groups not being converted into a prodrug functionality may be protected (for example, using a t-butyl-dimethylsilyl group), and later deprotected. Also, enzymatic methods may be used to selectively phosphorylate or dephosphorylate alcohol functionalities. One skilled in the art would readily know how to successfully carry out these as well as other known methods of esterification of alcohols.

Esters of the compounds of the invention may form salts. Where this is the case, this is achieved by conventional techniques as described above.

### Solvates

One or more compounds of the invention may exist in unsolvated as well as solvated forms with solvents such as water, ethanol, and the like, and it is intended that the invention embrace both solvated and unsolvated forms.

“Solvate” means a physical association of a compound of this invention with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Solvates for use in pharmaceutical compositions will be pharmaceutically acceptable esters, but other solvates may be useful in the production of the compounds of the invention.

As used herein, the term “pharmaceutically acceptable solvates” means solvates of compounds of the present invention that are pharmacologically acceptable and substantially non-toxic to the subject to which they are administered. More specifically, these solvates retain the biological effectiveness and properties of the compounds of the invention and are formed from suitable non-toxic solvents.

Non-limiting examples of suitable solvates include ethanolates, methanolates, and the like, as well as hydrates, which are solvates wherein the solvent molecules are H₂O.

Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature and cooling the solution at a rate sufficient to form crystals which are then isolated by standard methods. Analytical techniques such as, for example infrared spectroscopy, show the presence of the solvent (or water) in the crystals as a solvate (or hydrate).

### Isomers and Tautomers

As used herein, the term “isomers” used in relation to compounds of the invention refers to the optical, geometric, and positional isomers of these compounds.

As used herein, the term “optical isomers” of compounds of the invention refers to racemates, enantiomers, and diastereoisomers of these compounds and mixtures thereof.

Indeed, some of the compounds of the invention have at least one asymmetric carbon atoms and can therefore exist in the form of optically pure enantiomers, as racemates and as mixture thereof. Some of the compounds have at least two asymmetric carbon atoms and can therefore exist in the form of pure diastereoisomers and as mixtures thereof. The synthesis of optically active forms may be carried out by standard techniques of organic chemistry well known in the art, for example by resolution of the racemic form by recrystallisation techniques, by chiral synthesis, by enzymatic resolution, by biotransformation or by chromatographic separation. More specifically, diastereomeric mixtures can be separated into their individual diastereomers on the basis of their physical chemical differences by methods well known to those skilled in the art, such as, for example, by chromatography and/or fractional crystallization. Enantiomers can be separated, for example, by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers.

In addition, the present invention embraces all geometric and positional isomers. For example, if a compound of the invention incorporates a double bond or a fused ring, both the cis- and trans-forms, as well as mixtures, are embraced within the scope of the invention.

Within the present invention it is to be understood that a compound of the invention may exhibit the phenomenon of tautomerism and that the formulae drawings within this specification can represent only one of the possible tautomeric forms. It is to be understood that the invention encompasses any tautomeric form and is not to be limited merely to any one tautomeric form utilized within the formulae drawings.

It is also to be understood that certain compounds may exhibit polymorphism, and that the invention encompasses all such forms.

### Methods and Uses of the Compounds of the Invention and Compositions

In another aspect, the present disclosure relates to a method for inhibiting RAS (wild type or mutant), for example HRAS, NRAS, and/or KRAS, preferably inhibiting HRAS, and more preferably selectively inhibiting HRAS (or in alternative embodiments selectively inhibiting NRAS, or in yet other alternative embodiments selectively inhibiting KRAS) in a subject in need thereof comprising administering to the subject an effective amount of a compound of formula (I), salt, ester, solvate, isomer, or tautomer thereof or composition disclosed herein. The present disclosure also relates to the use of a compound of formula (I), salt, ester, solvate, isomer, or tautomer thereof or composition disclosed herein for inhibiting RAS (wild type or mutant), for example HRAS, NRAS, and/or KRAS, preferably inhibiting HRAS, and more preferably selectively inhibiting HRAS (or in alternative embodiments selectively inhibiting NRAS, or in yet other alternative embodiments selectively inhibiting KRAS) in a subject, or for the manufacture of a medicament for inhibiting HRAS in a subject. The present disclosure also relates to a compound of formula (I), salt, ester, solvate, isomer, or tautomer thereof or composition disclosed herein for use in inhibiting RAS (wild type or mutant), for example HRAS, NRAS, and/or KRAS, preferably inhibiting HRAS, and more preferably selectively inhibiting HRAS (or in alternative embodiments selectively inhibiting NRAS, or in yet other alternative embodiments selectively inhibiting KRAS) in a subject.

In embodiments, the above method, use and compound are for the prevention or treatment of a disease or disorder associated with abnormal RAS activity, for example abnormal RAS activity caused by a mutation in RAS. Examples of disease or disorder associated with abnormal HRAS activity, such as abnormal HRAS activity include Costello syndrome, epidermal nevus (e.g., epidermal nevus sebaceous), giant congenital melanocytic nevus, Noonan syndrome, Noonan syndrome with multiple lentigines, autoimmune lymphoproliferative syndrome, cardio-facio-cutaneous syndrome, neurofibromatosis type 1, capillary malformation-arteriovenous malformation syndrome, Legius syndrome as well as several types of cancers.

In another aspect, the present disclosure relates to a method for treating cancer in a subject in need thereof comprising administering to the subject an effective amount of a compound of formula (I), salt, ester, solvate, isomer, or tautomer thereof or composition disclosed herein. The present disclosure also relates to the use of a compound of formula (I), salt, ester, solvate, isomer, or tautomer thereof or composition disclosed herein for treating cancer in a subject, or for the manufacture of a medicament for treating cancer in a subject. The present disclosure also relates to a compound of formula (I), salt, ester, solvate, isomer, or tautomer thereof or composition disclosed herein for use in treating cancer in a subject.

### HRAS

In embodiments, the above method, use and compound are for selectively inhibiting HRAS. In embodiments, the HRAS is wild type HRAS or mutant HRAS.

In embodiments, the above method, use and compound are for inhibiting HRAS mutant, such as HRAS mutated at residue 12.

In embodiments, the above method, use and compound are for selectively inhibiting HRAS mutated at position 12.

In embodiments, the above method, use and compound are for inhibiting HRAS G12V mutant.

In embodiments, the above method, use and compound are for selectively inhibiting HRAS G12V mutant.

In embodiments, the above method, use and compound are for the prevention or treatment of a disease or disorder associated with abnormal HRAS activity, for example abnormal HRAS activity caused by a mutation in HRAS. In a further embodiment, the mutation in HRAS is a mutation at position 12. Examples of disease or disorder associated with abnormal HRAS activity, such as abnormal HRAS activity caused by a mutation at residue 12 of HRAS include Costello syndrome, epidermal nevus (e.g., epidermal nevus sebaceous) as well as several types of cancers.

In preferred embodiments, the above method, use, and compound are for treating a cancer with a mutated HRAS. In embodiments, the cancer is bladder, breast, colon, colorectal, cutaneous, embryonal rhabdomyosarcoma, endometrial, glioblastoma, head and neck, leukemia, lung, melanoma (including cutaneous melanoma), oral cavity, ovarian, prostate, renal, salivary duct, skin, or thyroid cancer. In preferred embodiments, the cancer is head and neck cancer (preferably head and neck squamous cell carcinoma), thyroid cancer, epithelial-myoepithelial carcinoma, kidney cancer or bladder cancer.

### NRAS

In embodiments, the above method, use and compound are for selectively inhibiting NRAS. In embodiments, the NRAS is wild type NRAS or mutant NRAS.

In embodiments, the above method, use and compound are for inhibiting NRAS mutant, such as NRAS mutated at residue 61.

In embodiments, the above method, use and compound are for selectively inhibiting NRAS mutated at position 61.

In embodiments, the above method, use and compound are for inhibiting NRAS Q61R mutant.

In embodiments, the above method, use and compound are for selectively inhibiting NRAS Q61R mutant.

In embodiments, the above method, use and compound are for the prevention or treatment of a disease or disorder associated with abnormal NRAS activity, for example abnormal NRAS activity caused by a mutation in NRAS. In a further embodiment, the mutation in NRAS is a mutation at position 61. Examples of disease or disorder associated with abnormal NRAS activity, such as abnormal NRAS activity caused by a mutation at residue 61 of NRAS include giant congenital melanocytic nevus, Noonan syndrome, autoimmune lymphoproliferative syndrome, epidermal nevus as well as several types of cancers.

In preferred embodiments, the above method, use, and compound are for treating a cancer with a mutated NRAS. In embodiments, the cancer is cancer is a melanoma (including individuals without giant congenital melanocytic nevus), lung cancer, cholangiocarcinoma, or a hematopoietic malignancy such core binding factor acute myeloid leukemia and cytogenetically normal acute myeloid leukemia. In preferred embodiments, the cancer is a melanoma (including individuals without giant congenital melanocytic nevus), lung cancer or a hematopoietic malignancy.

### KRAS

In embodiments, the above method, use and compound are for selectively inhibiting KRAS. In embodiments, the KRAS is wild type KRAS or mutant KRAS.

In embodiments, the above method, use and compound are for inhibiting KRAS mutant, such as KRAS mutated at residue 12.

In embodiments, the above method, use and compound are for selectively inhibiting KRAS mutated at position 12.

In embodiments, the above method, use and compound are for inhibiting KRAS G12C or G12D mutant.

In embodiments, the above method, use and compound are for selectively inhibiting KRAS G12C or G12D mutant.

In embodiments, the above method, use and compound are for the prevention or treatment of a disease or disorder associated with abnormal KRAS activity, for example abnormal KRAS activity caused by a mutation in KRAS. In a further embodiment, the mutation in KRAS is a mutation at position 12. Examples of disease or disorder associated with abnormal KRAS activity, such as abnormal KRAS activity caused by a mutation at residue 12 of KRAS include cardio-facio-cutaneous syndrome, Noonan syndrome, autoimmune lymphoproliferative syndrome, Epidermal nevus, as well as several types of cancers (oncogenic KRAS mutations have been identified in approximately 30% of human cancers).

In preferred embodiments, the above method, use, and compound are for treating a cancer with a mutated KRAS. In embodiments, the cancer is pancreatic cancer, cholangiocarcinoma, Core binding factor acute myeloid leukemia, colorectal cancer, or lung cancer (including non-small cell lung cancer). In preferred embodiments, the cancer is pancreatic cancer, colorectal cancer, or a lung cancer.

### Pan-RAS

In embodiments, the above method, use and compound are for selectively inhibiting pan-RAS (i.e., any combination or all of HRAS, NRAS and KRAS). In embodiments, the pan-RAS is wild type pan-RAS or mutant pan-RAS.

In embodiments, the above method, use and compound are for inhibiting a PAN-RAS mutant, such as PAN-RAS mutated at residue 12.

In embodiments, the above method, use and compound are for selectively inhibiting PAN-RAS mutated at position 12.

In embodiments, the above method, use and compound are for inhibiting PAN-RAS G12C or G12D mutant, preferably PAN-RAS G12C mutant.

In embodiments, the above method, use and compound are for selectively inhibiting PAN-RAS G12C or G12D mutant, preferably PAN-RAS G12C mutant.

In embodiments, the above method, use and compound are for the prevention or treatment of a disease or disorder associated with abnormal PAN-RAS activity, for example abnormal PAN-RAS activity caused by a mutation in PAN-RAS. In a further embodiment, the mutation in PAN-RAS is a mutation at position 12. Examples of disease or disorder associated with abnormal PAN-RAS activity, such as abnormal PAN-RAS activity caused by a mutation at residue 12 of PAN-RAS include those listed above for HRAS, NRAS, and KRAS.

In preferred embodiments, the above method, use, and compound are for treating a cancer with a mutated PAN-RAS. In embodiments, the cancer is any of those listed above for HRAS, NRAS, and KRAS.

### Dosage and Administration

A compound of formula (I) or salt, ester, solvate, isomer, or tautomer thereof or composition disclosed herein may be used alone or in combination with other therapies for the treatment of the above-noted disease or condition.

In an embodiment, the above-mentioned treatment comprises the use/administration of more than one (i.e., a combination of) active/therapeutic agent or therapy, one of which being the above-mentioned compound of formula I or salt, ester, solvate, isomer, or tautomer thereof. The combination of therapeutic agents or therapies may be administered or co-administered (e.g., consecutively, simultaneously, at different times) in any conventional manner. Co-administration in the context of the present disclosure refers to the administration of more than one therapy in the course of a coordinated treatment to achieve an improved clinical outcome. Such co-administration may also be coextensive, that is, occurring during overlapping periods of time. For example, a first therapy may be administered to a patient before, concomitantly, before and after, or after a second therapy is administered. In the case of a combination of active agents, they may be combined/formulated in a single composition and thus administered at the same time.

In an embodiment, the compound of formula I or salt, ester, solvate, isomer, or tautomer thereof is used in combination with one or more therapies for the treatment of cancer, e.g., chemotherapy, immunotherapy (e.g., CAR T/NK cell therapy, antibody-based therapy, checkpoint inhibitor therapy), surgery, radiotherapy, etc.

Any suitable amount of the pharmaceutical composition may be administered to a subject. The dosages will depend on many factors including the mode of administration. Typically, the amount of the compound of formula I or salt, ester, solvate, isomer, or tautomer thereof contained within a single dose will be an amount that effectively prevent, delay or treat the above-noted disease or condition (e.g., cancer) without inducing significant toxicity.

For the prevention, treatment or reduction in the severity of a given disease or condition, the appropriate dosage of the compound/composition will depend on the type of disease or condition to be treated, the severity and course of the disease or condition, whether the compound/composition is administered for preventive or therapeutic purposes, previous therapy, the patient's clinical history and response to the compound/composition, and the discretion of the attending physician. The compound/composition is suitably administered to the patient at one time or over a series of treatments. Preferably, it is desirable to determine the dose-response curve in vitro, and then in useful animal models prior to testing in humans. The present invention provides dosages for the compounds and compositions comprising same. For example, depending on the type and severity of the disease, about 1 μg/kg to to 1000 mg per kg (mg/kg) of body weight per day. Further, the effective dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, and may increase by 25 mg/kg increments up to 1000 mg/kg, or may range between any two of the foregoing values. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

These are simply guidelines since the actual dose must be carefully selected and titrated by the attending physician based upon clinical factors unique to each patient or by a nutritionist. The optimal daily dose will be determined by methods known in the art and will be influenced by factors such as the age of the patient and other clinically relevant factors. In addition, patients may be taking medications for other diseases or conditions. The other medications may be continued during the time that * is given to the patient, but it is particularly advisable in such cases to begin with low doses to determine if adverse side effects are experienced.

### Compositions Comprising the Compounds of the Invention

In another aspect, the present invention provides a composition comprising the above-mentioned compound and a carrier or excipient, in a further embodiment a pharmaceutically acceptable carrier or excipient. Such compositions may be prepared in a manner well known in the pharmaceutical art. Supplementary active compounds can also be incorporated into the compositions. The carrier/excipient can be suitable, for example, for intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press). Therapeutic formulations are prepared using standard methods known in the art by mixing the active ingredient having the desired degree of purity with one or more optional pharmaceutically acceptable carriers, excipients and/or stabilizers.

An “excipient,” as used herein, has its normal meaning in the art and is any ingredient that is not an active ingredient (drug) itself. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present system is not limited in these respects. In certain embodiments, one or more formulations of the dosage form include excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive.

Useful diluents, e.g., fillers, include, for example and without limitation, dicalcium phosphate, calcium diphosphate, calcium carbonate, calcium sulfate, lactose, cellulose, kaolin, sodium chloride, starches, powdered sugar, colloidal silicon dioxide, titanium oxide, alumina, talc, colloidal silica, microcrystalline cellulose, silicified micro crystalline cellulose and combinations thereof. Fillers that can add bulk to tablets with minimal drug dosage to produce tablets of adequate size and weight include croscarmellose sodium NF/EP (e.g., Ac-Di-Sol); anhydrous lactose NF/EP (e.g., Pharmatose™ DCL 21); and/or povidone USP/EP.

Binder materials include, for example and without limitation, starches (including corn starch and pregelatinized starch), gelatin, sugars (including sucrose, glucose, dextrose and lactose), polyethylene glycol, povidone, waxes, and natural and synthetic gums, e.g., acacia sodium alginate, polyvinylpyrrolidone, cellulosic polymers (e.g., hydroxypropyl cellulose, hydroxypropyl methylcellulose, methyl cellulose, hydroxyethyl cellulose, carboxymethylcellulose, colloidal silicon dioxide NF/EP (e.g., Cab-O-Si™ M5P), Silicified Microcrystalline Cellulose (SMCC), e.g., Silicified microcrystalline cellulose NF/EP (e.g., Prosolv™ SMCC 90), and silicon dioxide, mixtures thereof, and the like), veegum, and combinations thereof.

Useful lubricants include, for example, canola oil, glyceryl palmitostearate, hydrogenated vegetable oil (type I), magnesium oxide, magnesium stearate, mineral oil, poloxamer, polyethylene glycol, sodium lauryl sulfate, sodium stearate fumarate, stearic acid, talc and, zinc stearate, glyceryl behenate, magnesium lauryl sulfate, boric acid, sodium benzoate, sodium acetate, sodium benzoate/sodium acetate (in combination), DL-leucine, calcium stearate, sodium stearyl fumarate, mixtures thereof, and the like.

Bulking agents include, for example: microcrystalline cellulose, for example, AVICEL® (FMC Corp.) or EMCOCEL® (Mendell Inc.), which also has binder properties; dicalcium phosphate, for example, EMCOMPRESS® (Mendell Inc.); calcium sulfate, for example, COMPACTROL® (Mendell Inc.); and starches, for example, Starch 1500; and polyethylene glycols (CARBOWAX®).

Disintegrating or dissolution promoting agents include: starches, clays, celluloses, alginates, gums, crosslinked polymers, colloidal silicon dioxide, osmogens, mixtures thereof, and the like, such as crosslinked sodium carboxymethyl cellulose (AC-DI-SOL©), sodium croscarmellose, sodium starch glycolate (EXPLOTAB®, PRIMO JEL®) crosslinked polyvinylpolypyrrolidone (PLASONE-XL®), sodium chloride, sucrose, lactose and mannitol.

Anti-adherents and glidants employable in the core and/or a coating of the solid oral dosage form may include talc, starches (e.g., cornstarch), celluloses, silicon dioxide, sodium lauryl sulfate, colloidal silica dioxide, and metallic stearates, among others.

Examples of silica flow conditioners include colloidal silicon dioxide, magnesium aluminum silicate and guar gum.

Suitable surfactants include pharmaceutically acceptable non-ionic, ionic and anionic surfactants. An example of a surfactant is sodium lauryl sulfate. If desired, the pharmaceutical composition to be administered may also contain minor amounts of nontoxic auxiliary substances such as wetting or emulsifying agents, pH-buffering agents and the like, for example, sodium acetate, sorbitan monolaurate, triethanolamine sodium acetate, triethanolamine oleate, etc. If desired, flavoring, coloring and/or sweetening agents may be added as well.

Examples of stabilizing agents include acacia, albumin, polyvinyl alcohol, alginic acid, bentonite, dicalcium phosphate, carboxymethylcellulose, hydroxypropylcellulose, colloidal silicon dioxide, cyclodextrins, glyceryl monostearate, hydroxypropyl methylcellulose, magnesium trisilicate, magnesium aluminum silicate, propylene glycol, propylene glycol alginate, sodium alginate, carnauba wax, xanthan gum, starch, stearate(s), stearic acid, stearic monoglyceride and stearyl alcohol.

Examples of thickening agent can be for example talc USP/EP, a natural gum, such as guar gum or gum arabic, or a cellulose derivative such as microcrystalline cellulose NF/EP (e.g., Avicel™ PH 102), methylcellulose, ethylcellulose or hydroxyethylcellulose. A useful thickening agent is hydroxypropyl methylcellulose, an adjuvant which is available in various viscosity grades.

Examples of plasticizers include: acetylated monoglycerides; these can be used as food additives; alkyl citrates, used in food packaging, medical products, cosmetics and children toys; triethyl citrate (TEC); acetyl triethyl citrate (ATEC), higher boiling point and lower volatility than TEC; tributyl citrate (TBC); acetyl tributyl citrate (ATBC), compatible with PVC and vinyl chloride copolymers; trioctyl citrate (TOC), also used for gums and controlled release medicines; acetyl trioctyl citrate (ATOC), also used for printing ink; trihexyl citrate (THC), compatible with PVC, also used for controlled release medicines; acetyl trihexyl citrate (ATHC), compatible with PVC; butyryl trihexyl citrate (BTHC, trihexyl o-butyryl citrate), compatible with PVC; trimethyl citrate (TMC), compatible with PVC; alkyl sulphonic acid phenyl ester, polyethylene glycol (PEG) or any combination thereof. Optionally, the plasticizer can comprise triethyl citrate NF/EP.

Examples of permeation enhancers include: sulphoxides (such as dimethylsulphoxide, DMSO), azones (e.g., laurocapram), pyrrolidones (for example 2-pyrrolidone, 2P), alcohols and alkanols (ethanol, or decanol), glycols (for example propylene glycol and polyethylene glycol), surfactants and terpenes.

Formulations suitable for oral administration may include (a) liquid solutions, such as an effective amount of active agent(s)/composition(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds/compositions of the invention include ethylenevinyl acetate copolymer particles, osmotic pumps, implantable infusion systems, and liposomes. Formulations for inhalation may contain excipients, (e.g., lactose) or may be aqueous solutions containing, for example, polyoxyethylene-9-lauryl ether, glycocholate and deoxycholate, or may be oily solutions for administration in the form of nasal drops, or as a gel.

### General Definitions

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted. In contrast, the phrase “consisting of” excludes any unspecified element, step, ingredient, or the like. The phrase “consisting essentially of” limits the scope to the specified materials or steps and those that do not materially affect the basic and novel characteristic(s) of the invention.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Similarly, herein a general chemical structure, such as Formulas I to IV, with various substituents (R¹, R², etc.) and various radicals (alkyl, halogen atom, etc.) enumerated for these substituents is intended to serve as a shorthand method of referring individually to each and every molecule obtained by the combination of any of the radicals for any of the substituents. Each individual molecule is incorporated into the specification as if it were individually recited herein. Further, all subsets of molecules within the general chemical structures are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed.

No language in the specification should be construed as indicating any non-claimed element as essential to the practice of the invention.

Herein, the term “about” has its ordinary meaning. In embodiments, it may mean plus or minus 10% or plus or minus 5% of the numerical value qualified.

Unless otherwise defined, all technical and scientific terms used herein have the same meaning as commonly understood by one of ordinary skill in the art to which this invention belongs.

Other objects, advantages and features of the present invention will become more apparent upon reading of the following non-restrictive description of specific embodiments thereof, given by way of example only with reference to the accompanying drawings.

### Description of Illustrative Embodiments

The present invention is illustrated in further details by the following non-limiting examples.

Compounds #10002 to 10011 are commercially available from Key Organics®, Azepine®, Enamine®, and Net Chem®. They are provided as comparative examples.

These compounds are:

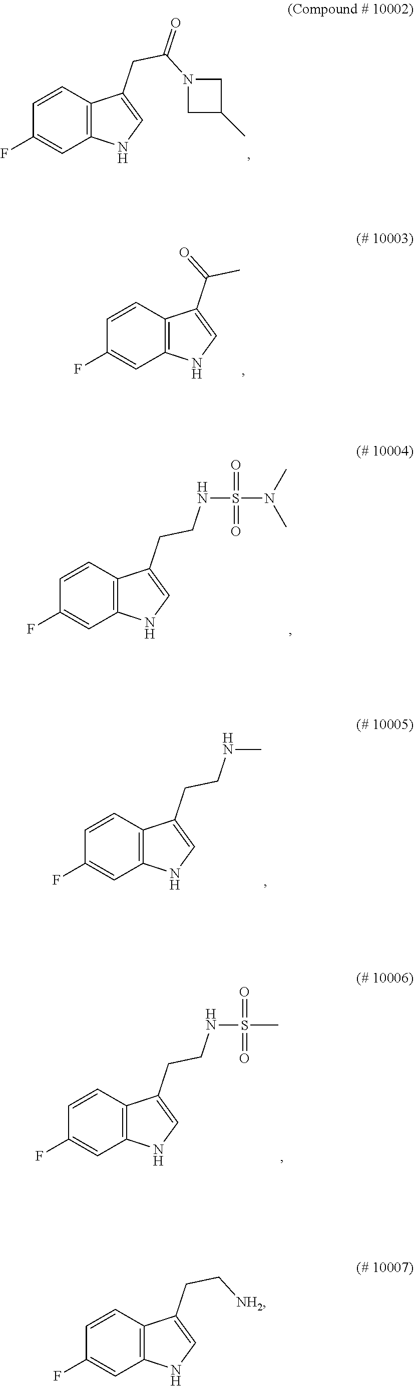 [SMILES: CC1CN(C(=O)Cc2c[nH]c3cc(F)ccc23)C1 ; CC(=O)c1c[nH]c2cc(F)ccc12 ; CN(C)S(=O)(=O)NCCc1c[nH]c2cc(F)ccc12 ; CNCCc1c[nH]c2cc(F)ccc12 ; CS(=O)(=O)NCCc1c[nH]c2cc(F)ccc12 ; NCCc1c[nH]c2cc(F)ccc12]

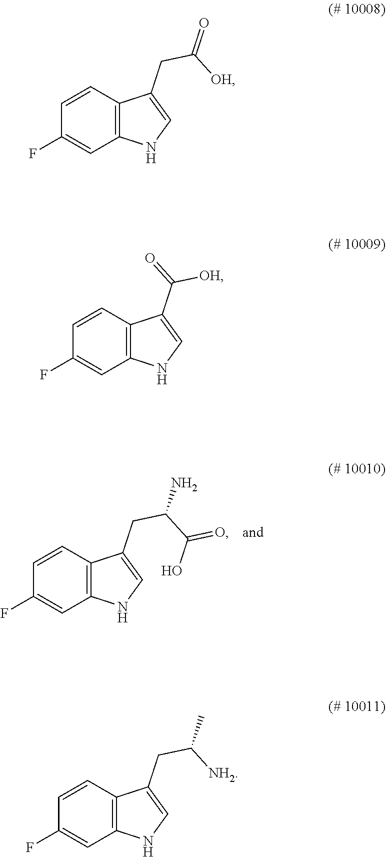 [SMILES: O=C(O)Cc1c[nH]c2cc(F)ccc12 ; O=C(O)c1c[nH]c2cc(F)ccc12 ; N[C@@H](Cc1c[nH]c2cc(F)ccc12)C(=O)O ; C[C@H](N)Cc1c[nH]c2cc(F)ccc12]

### Synthesis of Compounds and General Scheme for Amide Libraries

All commercially available reagents and anhydrous solvents were used without further purification. Purity assessment for final compounds based on analytical HPLC: 4.6 mm×50 mm Waters YMC Pro-C18, 5 μm column, 120 A. Mobile phases are as follows: A, H2O with 0.2% formic acid; B, acetonitrile with 0.2% formic acid. Gradient: 10-90% B in 3 min with a 5 min run time. The flow rate was 1.5 mL/min. Unless specified otherwise, all compounds were >95% pure. Mass samples were analyzed on a Micro Mass ZQ, ZMD, Quattro LC, or Quatro II mass spectrometer operated in a single MS mode with electrospray ionization. Samples were introduced into the mass spectrometer using flow injection (FIA) or chromatography. The mobile phase for all mass analysis consisted of acetonitrile-water mixtures with either 0.2% formic acid or ammonium formate. 1H NMR spectra were recorded using either a Bruker Avance 400 (400 MHz) or a Bruker Avance II—300 (300 MHz) instrument. Column chromatography was performed using Teledyne ISCO RediSep Normal Phase (35-70 μm) or RediSep Gold Normal Phase (25-40 μm) silica flash columns using a Teledyne ISCO Combiflash Companion or Combiflash Rf purification system. Preparative reversed-phase chromatography was carried out using a Gilson 215 liquid handler coupled to a UV-vis 156 Gilson detector and an Agilent Zorbax SB-C18 column, 21.2 mm×100 mm. A linear gradient from 10% to 90% CH3CN in H2O over 10 min (0.1% trifluoroacetic acid) was used; the flow rate was 20 mL/min.

High-resolution mass spectrometry data were collected on a Thermo Scientific QExactive mass spectrometer coupled to a Waters Acquity UPLC system. Samples were analyzed from a 100 μM DMSO solution with a 3 μL injection volume. The chromatographic column was a Waters Acquity CSH C18, 2.1×50 mm, 1.7 μm particle size. Gradient elution was employed using 0.1% formic acid in water as mobile phase A and 0.1% formic acid as mobile phase B. The gradient began at 10% B and increased to 60% B over 0.8 min and to 100% B over the next 0.2 min, followed by a 0.5 min re-equilibration at the initial conditions. The mass spectrometer was run in full MS mode, positive polarity, with the resolution set to 35 000. A heated electrospray source was used with settings of 3.5 kV and 400° C.

### Example 1—Synthesis of 2-(6-fluoro-1H-indol-3-yl)-N-phenyl acetamide (Compound #10021) (40-006 in Scheme 1)

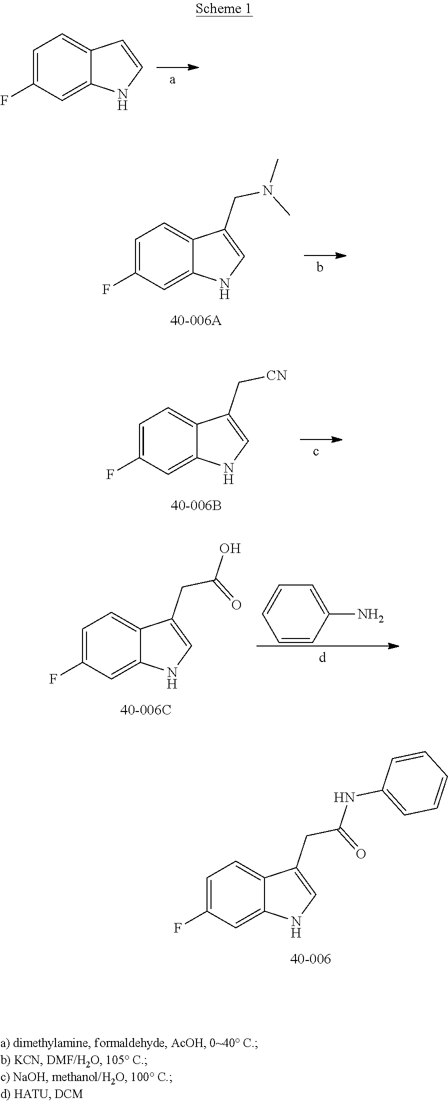

### 1-(6-fluoro-1H-indol-3-yl)-N,N-dimethylmethanamine (40-006A)

The solution of 40 percent aq. dimethylamine (9.1 g, 80.74 mmol) was cooled to 5° C., and glacial acetic acid (6.1 mL) was added dropwise while maintaining the temperature at ˜10° C. After stirring for 20 minutes, 37 percent aqueous formaldehyde (6.1 mL, 80.74 mmol) was slowly added to above solution while keeping the temperature between 0˜10° C. followed by addition of 6-Fluoroindole (10 g, 74.00 mmol). The reaction was exothermic and reached a final temperature˜40° C., and it was then cooled down to ˜20° C. The reaction solution was then slowly added to 160 mL of aqueous NaOH solution (3M). The suspension was stirred about 30 minutes, and then collected by filtration. The cake was rinsed with water (50 mL×2) and dried to afford yellow solid (12.1 g, >99%).

¹H NMR (400 MHz, CDCl₃) δ 8.22 (br s, 1H), 7.63-7.59 (m, 1H), 7.08-7.07 (m, 1H), 7.03-7.00 (m, 1H), 6.91-6.86 (m, 1H), 3.60 (s, 2H), 2.28 (s, 6H).

### 2-(6-fluoro-1H-indol-3-yl) acetonitrile (40-006B)

The solution of (6-fluoro-1H-indol-3-ylmethyl)-dimethylamine (12 g, 62.42 mmol), KCN (6.7 g, 102.89 mmol) in DMF (36 mL) and water (19 mL) were heated to 105° C. for 10 hours. After which period, the reaction mixture was cooled down to 25° C., water (145 mL) and toluene (80 mL) were added thereto and stirred for 3 hours. The organic and aqueous layers were separated. The organic layer was washed with aqueous sodium bicarbonate (80 mL) and brine (80 mL), dried over sodium sulfate. After filtration and concentrated, the residue was purified with flash column on silica gel to get desired product as yellow oil (5.7 g, 52.4% yield).

¹H NMR (400 MHz, CDCl₃) δ 8.19 (s, 1H), 7.52-7.49 (m, 1H), 7.22-7.21 (m, 1H), 7.08 (dd, J=9.4, 2.2 Hz, 1H), 6.98-6.93 (m, 1H), 3.82 (d, J=1.1 Hz, 2H).

### 2-(6-fluoro-1H-indol-3-yl) acetic acid (40-006C)

The mixture of 2-(6-fluoro-1H-indol-3-yl) acetonitrile (2 g, 11.48 mmol), sodium hydroxide (2.6 g, 65.00 mmol), methanol (15 mL) and water (45 mL) was stirred at 100° C. for 3 hours. Then, the reaction was cooled to 0° C. and treated with 6 N aqueous solution of HCl to pH-1. The solid formed was collected by filtration, which was then washed twice with water and dried to give title compound as yellow solid (1.7 g, 76.6% yield).

MS (ESI+) m/z 194 (M+H)⁺.

¹H NMR (400 MHz, DMSO-d₆) δ 12.18 (s, 1H), 10.97 (s, 1H), 7.50-7.46 (m, 1H), 7.23-7.22 (m, 1H), 7.12 (dd, J=10.2, 2.3 Hz, 1H), 6.87-6.82 (m, 1H), 3.63 (s, 2H).

### General Procedures I for amide Coupling Exemplified Here for the Production of: 2-(6-fluoro-1H-indol-3-yl)-N-phenyl Acetamide (40-006) (Compound #10021)

The solution of 2-(6-fluoro-1H-indol-3-yl) acetic acid (100 mg, 0.52 mmol), aniline (53.0 mg, 0.57 mmol), HATU (216.5 mg, 0.57 mmol) and Et₃N (68.1 mg, 0.67 mmol) in DCM (8 mL) was stirred at room temperature for 16 hours. After then, the mixture was diluted with dichloromethane (30 mL), which was washed with HCl (15 mL, 1.0 N) and brine (20 mL), dried over sodium sulfate. After filtration and concentration, the residue was purified by flash column on silica gel to obtain desired product as colorless oil (80 mg, 57.6% yield).

MS (ESI+) m/z 269 (M+H)⁺.

¹H NMR (400 MHz, DMSO-d6) δ 10.98 (s, 1H), 10.09 (s, 1H), 7.76-7.48 (m, 3H), 7.38-7.20 (m, 3H), 7.12 (dd, J=10.2, 2.3 Hz, 1H), 7.08-6.96 (m, 1H), 6.88-6.83 (m, 1H), 3.71 (s, 2H).

### Example 2—Synthesis of Further Compounds Using the Procedure of Example 1

Using the above procedures exemplified by Scheme 1, the following compounds were synthesized:

TABLE 1

| | Compound | | LC-MS |
|---|---|---|---|
| Structure | # | ¹H NMR | (M + H)⁺ |
| 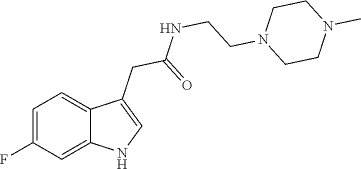 [SMILES: CN1CCN(CCNC(=O)Cc2c[nH]c3cc(F)ccc23)CC1] | 10020 | ¹H NMR (400 MHz, CDCl₃) δ 8.42 (s, 1H), 7.49-7.46 (m, 1H), 7.17-7.05 (m, 2H), 6.93-6.88 (m, 1H), 6.30 (s, 1H), 3.71 (s, 2H), 3.26 (dd, J = 11.2, 5.8 Hz, 2H), 2.48- 1.89 (m, 13H). | 319 |
| 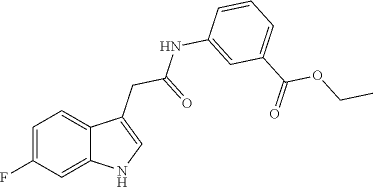 [SMILES: CCOC(=O)c1cccc(NC(=O)Cc2c[nH]c3cc(F)ccc23)c1] | 10019 | ¹H NMR (400 MHz, CDCl₃) δ 8.29 (s, 1H), 7.86 (d, J = 7.7 Hz, 1H), 7.75-7.73 (m, 2H), 7.52 (dd, J = 8.7, 5.2 Hz, 1H), 7.43 (s, 1H), 7.38-7.33 (m, 1H), 7.24-7.22 (m, 1H), 7.13 (dd, J = 9.4, 2.2 Hz, 1H), 6.98-6.89 (m, 1H), 4.33 (q, J = 7.1 Hz, 2H), 3.89 (s, 2H), 1.36 (t, J = 7.1 Hz, 3H). | 341 |
| 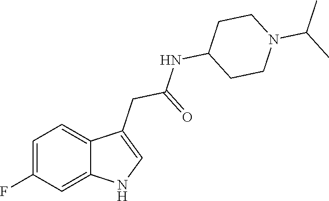 [SMILES: CC(C)N1CCC(NC(=O)Cc2c[nH]c3cc(F)ccc23)CC1] | 10018 | ¹H NMR (400 MHz, CDCl₃) δ 8.70 (s, 1H), 7.45-7.42 (m, 1H), 7.15-6.99 (m, 2H), 6.92-6.87 (m, 1H), 5.54 (d, J = 7.9 Hz, 1H), 3.80-3.75 (m, 1H), 3.68 (s, 2H), 2.68-2.65 (m, 3H), 2.22-2.16 (m, 2H), 1.85-1.81 (m, 2H), 1.26-1.16 (m, 2H), 0.96 (d, J = 6.6 Hz, 6H). | 318 |
| 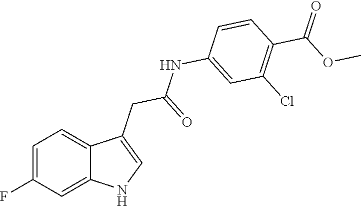 [SMILES: COC(=O)c1ccc(NC(=O)Cc2c[nH]c3cc(F)ccc23)cc1Cl] | 10017 | ¹H NMR (400 MHz, CDCl₃) δ 8.28 (s, 1H), 7.79 (d, J = 8.6 Hz, 1H), 7.55-7.48 (m, 2H), 7.44 (s, 1H), 7.35-7.32 (m, 1H), 7.24-7.22 (m, 1H), 7.14 (dd, J = 9.3, 2.2 Hz, 1H), 7.01-6.91 (m, 1H), 3.89-3.88 (m, 5H). | 361 |
| 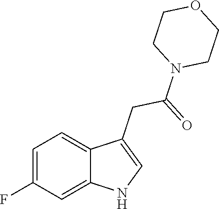 [SMILES: O=C(Cc1c[nH]c2cc(F)ccc12)N1CCOCC1] | 10016 | ¹H NMR (400 MHz, CDCl₃) δ 8.14 (s, 1H), 7.53 (dd, J = 8.7, 5.3 Hz, 1H), 7.06-7.02 (m, 2H), 6.92-6.87 (m, 1H), 3.82 (s, 2H), 3.65 (s, 4H), 3.49 (s, 4H). | 263 |
| 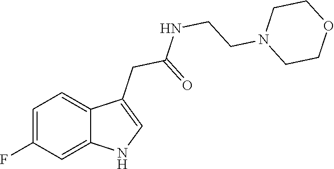 [SMILES: O=C(Cc1c[nH]c2cc(F)ccc12)NCCN1CCOCC1] | 10015 | ¹H NMR (400 MHz, CDCl₃) δ 8.49 (s, 1H), 7.48-7.45 (m, 1H), 7.14-7.02 (m, 2H), 6.93-6.88 (m, 1H), 6.32 (s, 1H), 3.72 (s, 2H), 3.51-3.18 (m, 6H), 2.32 (t, J = 6.0 Hz, 2H), 2.28-2.06 (m, 4H). | 306 |
| 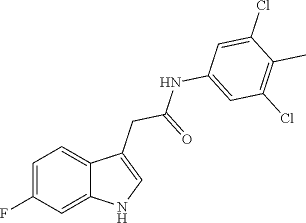 [SMILES: Cc1c(Cl)cc(NC(=O)Cc2c[nH]c3cc(F)ccc23)cc1Cl] | 10022 | ¹H NMR (400 MHz, CDCl₃) δ 8.28 (s, 1H), 7.51-7.48 (m, 1H), 7.35 (s, 2H), 7.21 (d, J = 1.9 Hz, 1H), 7.12 (dd, J = 9.4, 2.2 Hz, 1H), 6.98-6.93 (m, 1H), 3.86 (s, 2H), 2.36 (s, 3H). | 351 |
| 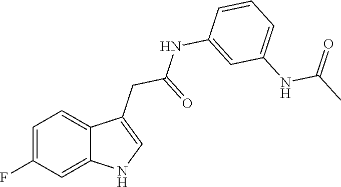 [SMILES: CC(=O)Nc1cccc(NC(=O)Cc2c[nH]c3cc(F)ccc23)c1] | 10014 | ¹H NMR (400 MHz, DMSO-d₆) δ 10.97 (s, 1H), 10.09 (s, 1H), 9.90 (s, 1H), 7.90 (s, 1H), 7.61-7.57 (m, 1H), 7.41-7.02 (m, 5H), 6.87-6.82 (m, 1H), 3.70 (s, 2H), 2.02 (s, 3H). | 326 |
| 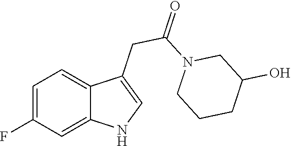 [SMILES: O=C(Cc1c[nH]c2cc(F)ccc12)N1CCCC(O)C1] | 10120 | ¹H NMR (400 MHz, CDCl₃) δ 8.33 (s, 1H), 7.52 (s, 1H), 7.11-6.97 (m, 2H), 6.90-6.85 (m, 1H), 3.95-3.17 (m, 7H), 1.91-1.20 (m, 4H). | 277 |
| 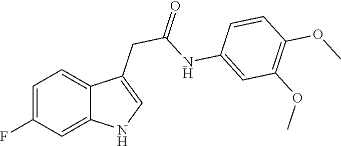 [SMILES: COc1ccc(NC(=O)Cc2c[nH]c3cc(F)ccc23)cc1OC] | 10033 | ¹H NMR (400 MHz, DMSO-d₆) δ 10.99- 10.95 (m, 1H), 9.95 (s, 1H), 7.63-7.56 (m, 1H), 7.33-7.30 (m, 1H), 7.26-7.23 (m, 1H), 7.14-7.06 (m, 2H), 6.91-6.81 (m, 2H), 3.70 (s, 6H), 3.67 (s, 2H). | 329 |
| 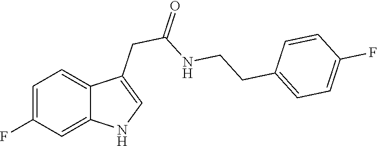 [SMILES: O=C(Cc1c[nH]c2cc(F)ccc12)NCCc1ccc(F)cc1] | 10031 | ¹H NMR (400 MHz, CDCl₃) δ 8.25 (s, 1H), 7.41-7.35 (m, 1H), 7.11-7.06 (m, 1H), 7.04-7.01 (m, 1H), 6.94-6.87 (m, 1H), 6.87-6.75 (m, 4H), 5.64-5.54 (m, 1H), 3.67 (s, 2H), 3.41 (q, J = 6.6 Hz, 2H), 2.64 (t, J = 6.8 Hz, 2H). | 315 |
| 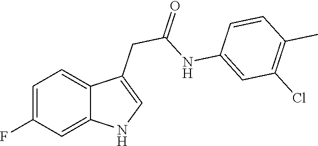 [SMILES: Cc1ccc(NC(=O)Cc2c[nH]c3cc(F)ccc23)cc1Cl] | 10032 | ¹H NMR (400 MHz, DMSO-d₆) δ 10.99 (s, 1H), 10.19 (s, 1H), 7.80 (d, J = 2.0 Hz, 1H), 7.61-7.53 (m, 1H), 7.40-7.34 (m, 1H), 7.29-7.22 (m, 2H), 7.15-7.08 (m, 1H), 6.90-6.81 (m, 1H), 3.70 (s, 2H), 2.25 (s, 3H). | 317 |
| 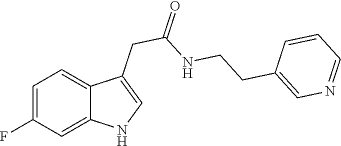 [SMILES: O=C(Cc1c[nH]c2cc(F)ccc12)NCCc1cccnc1] | 10029 | ¹H NMR (400 MHz, DMSO-d₆) δ 10.92 (s, 1H), 8.45-8.33 (m, 2H), 8.03-7.88 (m, 1H), 7.58-7.49 (m, 1H), 7.49-7.41 (m, 1H), 7.29-7.19 (m, 1H), 7.17-7.05 (m, 2H), 6.87-6.76 (m, 1H), 3.45 (s, 2H), 3.36- 3.25 (m, 2H), 2.71 (t, J = 7.0 Hz, 2H). | 298 |
| 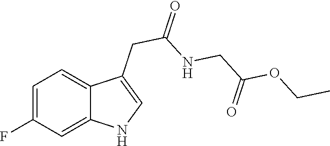 [SMILES: CCOC(=O)CNC(=O)Cc1c[nH]c2cc(F)ccc12] | 10050 | ¹H NMR (400 MHz, DMSO-d₆) δ 10.95 (s, 1H), 8.35-8.24 (m, 1H), 7.58-7.47 (m, 1H), 7.21 (d, J = 2.3 Hz, 1H), 7.11 (dd, J = 10.2, 2.3 Hz, 1H), 6.90-6.74 (m, 1H), 4.07 (q, J = 7.1 Hz, 2H), 3.81 (d, J = 5.9 Hz, 2H), 3.55 (s, 2H), 1.16 (t, J = 7.1 Hz, 3H). | 279 |
| 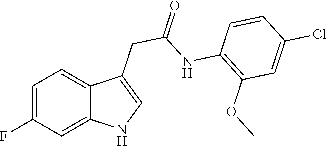 [SMILES: COc1cc(Cl)ccc1NC(=O)Cc1c[nH]c2cc(F)ccc12] | 10051 | ¹H NMR (400 MHz, DMSO-d₆) δ 11.03 (s, 1H), 9.21 (s, 1H), 8.11 (d, J = 2.5 Hz, 1H), 7.64-7.53 (m, 1H), 7.30 (s, 1H), 7.14 (dd, J = 10.2, 2.3 Hz, 1H), 7.10-7.05 (m, 1H), 7.04-6.99 (m, 1H), 6.91-6.83 (m, 1H), 3.85 (s, 2H), 3.77 (s, 3H). | 333 |
| 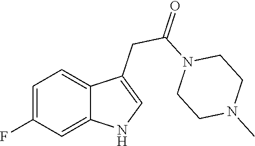 [SMILES: CN1CCN(C(=O)Cc2c[nH]c3cc(F)ccc23)CC1] | 10065 | ¹H NMR (400 MHz, DMSO-d₆) δ 10.95 (s, 1H), 7.56-7.48 (m, 1H), 7.22-7.16 (m, 1H), 7.10 (dd, J = 10.2, 2.3 Hz, 1H), 6.87- 6.78 (m, 1H), 3.74 (s, 2H), 3.51-3.40 (m, 4H), 2.22-2.09 (m, 7H). | 276 |
| 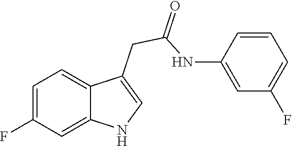 [SMILES: O=C(Cc1c[nH]c2cc(F)ccc12)Nc1cccc(F)c1] | 10072 | ¹H NMR (400 MHz, DMSO-d₆) δ 11.00 (s, 1H), 10.33 (s, 1H), 7.65-7.54 (m, 2H), 7.37-7.25 (m, 3H), 7.14 (dd, J = 10.2, 2.3 Hz, 1H), 6.91-6.80 (m, 2H), 3.74 (s, 2H). | 287 |
| 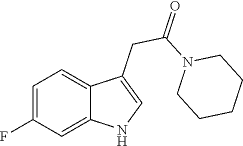 [SMILES: O=C(Cc1c[nH]c2cc(F)ccc12)N1CCCCC1] | 10053 | ¹H NMR (400 MHz, CDCl₃) δ 8.17 (s, 1H), 7.58-7.51 (m, 1H), 7.07-6.99 (m, 2H), 6.93-6.84 (m, 1H), 3.85-3.78 (m, 2H), 3.66-3.36 (m, 4H), 1.63-1.33 (m, 6H). | 261 |
| 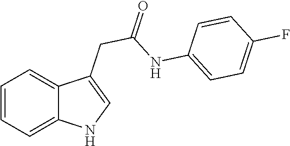 [SMILES: O=C(Cc1c[nH]c2ccccc12)Nc1ccc(F)cc1] | 10067 | ¹H NMR (400 MHz, DMSO-d₆) δ 10.91 (s, 1H), 10.14 (s, 1H), 7.70-7.52 (m, 3H), 7.37-7.32 (m, 1H), 7.27-7.23 (m, 1H), 7.16-6.95 (m, 4H), 3.71 (s, 2H). | 269 |
| 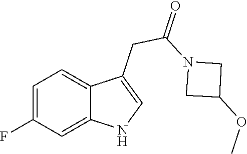 [SMILES: COC1CN(C(=O)Cc2c[nH]c3cc(F)ccc23)C1] | 10081 | ¹H NMR (400 MHz, CDCl₃) δ 8.23 (s, 1H), 7.55-7.49 (m, 1H), 7.10-7.06 (m, 1H), 7.04-6.98 (m, 1H), 6.94-6.84 (m, 1H), 4.29-4.10 (m, 3H), 4.03-3.87 (m, 2H), 3.58 (s, 2H), 3.27 (s, 3H). | 263 |
| 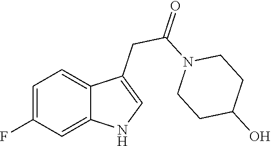 [SMILES: O=C(Cc1c[nH]c2cc(F)ccc12)N1CCC(O)CC1] | 10086 | ¹H NMR (400 MHz, DMSO-d₆) δ 10.95 (s, 1H), 7.58-7.49 (m, 1H), 7.22-7.16 (m, 1H), 7.15-7.05 (m, 1H), 6.89-6.75 (m, 1H), 4.66 (d, J = 4.1 Hz, 1H), 3.99-3.86 (m, 1H), 3.84-3.68 (m, 3H), 3.65-3.55 (m, 1H), 3.21-3.09 (m, 1H), 3.01-2.89 (m, 1H), 1.69-1.49 (m, 2H), 1.23-1.02 (m, 2H). | 277 |
| 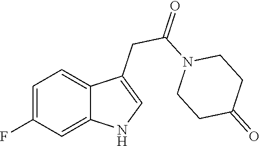 [SMILES: O=C1CCN(C(=O)Cc2c[nH]c3cc(F)ccc23)CC1] | 10106 | ¹H NMR (400 MHz, DMSO-d₆) δ 10.98 (s, 1H), 7.60-7.53 (m, 1H), 7.28-7.22 (m, 1H), 7.14-7.08 (m, 1H), 6.88-6.80 (m, 1H), 3.86 (s, 2H), 3.83-3.69 (m, 4H), 2.35- 2.20 (m, 4H). | 275 |
| 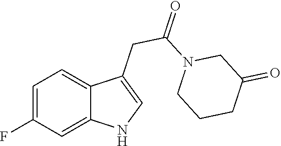 [SMILES: O=C1CCCN(C(=O)Cc2c[nH]c3cc(F)ccc23)C1] | 10109 | ¹H NMR (400 MHz, CDCl₃) δ 8.21 (br s, 1H), 7.60-7.48 (m, 1H), 7.12-6.97 (m, 2H), 6.95-6.83 (m, 1H), 4.23 (s, 1H), 4.06 (s, 1H), 3.90-3.64 (m, 4H), 2.47-2.36 (m, 2H), 2.01-1.83 (m, 2H). | 275 |
| 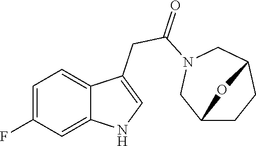 [SMILES: O=C(Cc1c[nH]c2cc(F)ccc12)N1C[C@H]2CC[C@@H](C1)O2] | 10122 | ¹H NMR (400 MHz, CDCl₃) δ 8.25 (s, 1H), 7.59-7.51 (m, 1H), 7.07-6.99 (m, 2H), 6.94-6.84 (m, 1H), 4.41-4.31 (m, 1H), 4.27-4.14 (m, 2H), 3.83-3.71 (m, 2H), 3.57-3.46 (m, 1H), 3.40-3.28 (m, 1H), 3.00-2.91 (m, 1H), 1.90-1.56 (m, 3H), 1.31-1.18 (m, 1H). | 289 |
| 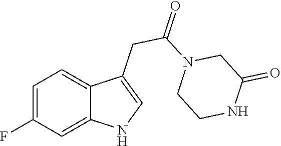 [SMILES: O=C1CN(C(=O)Cc2c[nH]c3cc(F)ccc23)CCN1] | 10145 | ¹H NMR (400 MHz, DMSO-d₆) δ 10.99 (s, 1H), 8.11-7.93 (m, 1H), 7.58-7.47 (m, 1H), 7.26-7.16 (m, 1H), 7.16-7.07 (m, 1H), 6.90-6.75 (m, 1H), 4.09 (s, 1H), 3.94 (s, 1H), 3.83-3.74 (m, 2H), 3.73-3.65 (m, 1H), 3.64-3.57 (m, 1H), 3.17-3.05 (m, 2H). | 276 |
| 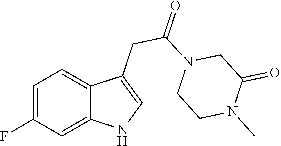 [SMILES: CN1CCN(C(=O)Cc2c[nH]c3cc(F)ccc23)CC1=O] | 10146 | ¹H NMR (400 MHz, CDCl₃) δ 8.57-8.37 (m, 1H), 7.61-7.46 (m, 1H), 7.11-6.81 (m, 3H), 4.28 (s, 0.5H), 4.18 (s, 1.5H), 3.91- 3.77 (m, 3.5H), 3.75-3.66 (m, 0.5H), 3.37-3.28 (m, 1.5H), 3.22-3.13 (m, 0.5H), 3.00-2.90 (m, 3H). | 290 |
| 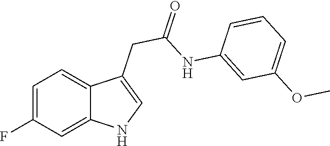 [SMILES: COc1cccc(NC(=O)Cc2c[nH]c3cc(F)ccc23)c1] | 10030 | ¹H NMR (400 MHz, CDCl₃) δ 8.27 (s, 1H), 7.57-7.50 (m, 1H), 7.32 (s, 1H), 7.24- 7.20 (m, 1H), 7.18-7.08 (m, 3H), 6.97- 6.89 (m, 1H), 6.79-6.75 (m, 1H), 6.65- 6.60 (m, 1H), 3.86 (s, 2H), 3.76 (s, 3H). | 299 |
| 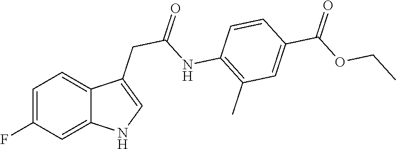 [SMILES: CCOC(=O)c1ccc(NC(=O)Cc2c[nH]c3cc(F)ccc23)c(C)c1] | 10034 | ¹H NMR (400 MHz, DMSO-d₆) δ 11.01 (s, 1H), 9.42 (s, 1H), 7.82-7.72 (m, 3H), 7.66- 7.56 (m, 1H), 7.35-7.25 (m, 1H), 7.21- 7.09 (m, 1H), 6.94-6.82 (m, 1H), 4.34- 4.22 (m, 2H), 3.82 (s, 2H), 2.20 (s, 3H), 1.30 (t, J = 7.1 Hz, 3H). | 355 |
| 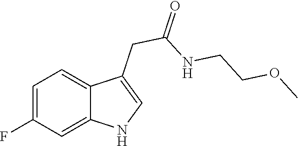 [SMILES: COCCNC(=O)Cc1c[nH]c2cc(F)ccc12] | 10025 | ¹H NMR (400 MHz, CDCl₃) δ 8.40-8.29 (m, 1H), 7.51-7.43 (m, 1H), 7.12-7.03 (m, 2H), 6.95-6.86 (m, 1H), 6.07-5.97 (m, 1H), 3.71 (s, 2H), 3.43-3.33 (m, 4H), 3.20 (s, 3H). | 251 |
| 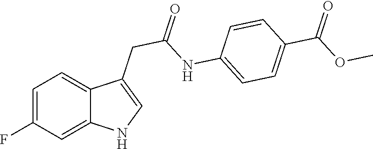 [SMILES: COC(=O)c1ccc(NC(=O)Cc2c[nH]c3cc(F)ccc23)cc1] | 10048 | ¹H NMR (400 MHz, CDCl₃) δ 8.28 (s, 1H), 7.96-7.91 (m, 2H), 7.56-7.45 (m, 2H), 7.45-7.41 (m, 2H), 7.24-7.22 (m, 1H), 7.15-7.10 (m, 1H), 6.99-6.92 (m, 1H), 4.33 (q, J = 7.1 Hz, 2H), 3.90 (s, 2H), 1.37 (t, J = 7.1 Hz, 3H). | 341 |
| 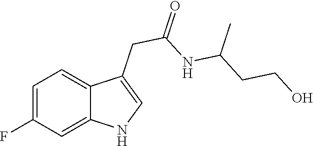 [SMILES: CC(CCO)NC(=O)Cc1c[nH]c2cc(F)ccc12] | 10026 | ¹H NMR (400 MHz, CDCl₃) δ 8.41 (s, 1H), 7.48-7.42 (m, 1H), 7.17-7.05 (m, 2H), 6.97-6.89 (m, 1H), 5.69-5.55 (m, 1H), 4.26-4.12 (m, 1H), 3.73 (s, 2H), 3.62- 3.43 (m, 2H), 1.81-1.70 (m, 1H), 1.28- 1.14 (m, 1H), 1.06 (d, J = 6.7 Hz, 3H). | 265 |
| 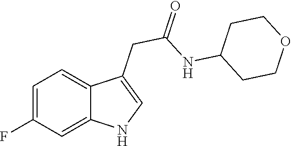 [SMILES: O=C(Cc1c[nH]c2cc(F)ccc12)NC1CCOCC1] | 10027 | ¹H NMR (400 MHz, CDCl₃) δ 8.33-8.21 (m, 1H), 7.48-7.41 (m, 1H), 7.17-7.06 (m, 2H), 6.95-6.90 (d, J = 1.6 Hz, 1H), 5.64-5.52 (m, 1H), 4.05-3.94 (m, 1H), 3.91-3.78 (m, 2H), 3.47-3.35 (m, 2H), 2.10-1.88 (m, 2H), 1.84-1.72 (m, 2H), 1.32-1.19 (m, 2H). | 277 |
| 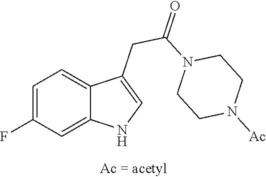 [SMILES: CC(=O)N1CCN(C(=O)Cc2c[nH]c3cc(F)ccc23)CC1] | 10040 | ¹H NMR (400 MHz, DMSO-d₆) δ 10.97 (s, 1H), 7.54 (s, 1H), 7.29-7.06 (m, 2H), 6.95- 6.74 (m, 1H), 3.79 (s, 2H), 3.65-3.43 (m, 8H), 1.98 (s, 3H). | 304 |
| 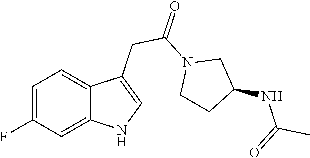 [SMILES: CC(=O)N[C@H]1CCN(C(=O)Cc2c[nH]c3cc(F)ccc23)C1] | 10041 | ¹H NMR (400 MHz, CDCl₃) δ 8.46-8.31 (m, 1H), 7.60-7.51 (m, 1H), 7.10-6.99 (m, 2H), 6.92-6.85 (m, 1H), 5.85-5.81 (m, 0.5H), 5.76-5.69 (m, 0.5H), 4.48- 4.36 (m, 1H), 3.79-3.65 (m, 3H), 3.64- 3.52 (m, 2H), 3.44-3.34 (m, 1H), 2.24- 2.00 (m, 1H), 1.94-1.86 (m, 3H), 1.82- 1.72 (m, 1H). | 304 |
| 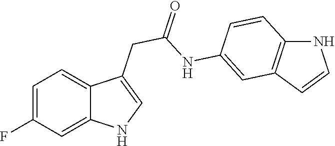 [SMILES: O=C(Cc1c[nH]c2cc(F)ccc12)Nc1ccc2[nH]ccc2c1] | 10042 | ¹H NMR (400 MHz, DMSO-d₆) δ 10.96 (s, 2H), 9.87 (s, 1H), 7.85 (s, 1H), 7.69-7.58 (m, 1H), 7.31-7.23 (m, 3H), 7.23-7.16 (m, 1H), 7.16-7.08 (m, 1H), 6.93-6.79 (m, 1H), 6.34 (s, 1H), 3.70 (s, 2H). | 308 |
| 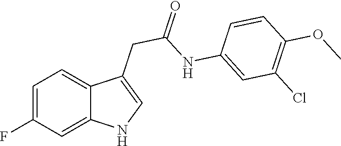 [SMILES: COc1ccc(NC(=O)Cc2c[nH]c3cc(F)ccc23)cc1Cl] | 10055 | ¹H NMR (400 MHz, DMSO-d₆) δ 11.01- 10.95 (m, 1H), 10.10 (s, 1H), 7.78 (d, J = 2.5 Hz, 1H), 7.60-7.54 (m, 1H), 7.46- 7.41 (m, 1H), 7.26-7.23 (m, 1H), 7.15- 7.06 (m, 2H), 6.89-6.82 (m, 1H), 3.80 (s, 3H), 3.68 (s, 2H). | 333 |
| 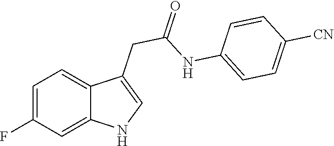 [SMILES: N#Cc1ccc(NC(=O)Cc2c[nH]c3cc(F)ccc23)cc1] | 10054 | ¹H NMR (400 MHz, DMSO-d₆) δ 11.01 (s, 1H), 10.55 (s, 1H), 7.84-7.73 (m, 4H), 7.60-7.53 (m, 1H), 7.29-7.25 (m, 1H), 7.15-7.10 (m, 1H), 6.90-6.82 (m, 1H), 3.77 (s, 2H). | 294 |
| 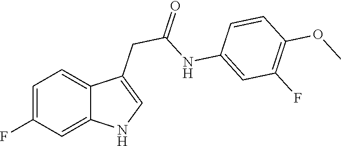 [SMILES: COc1ccc(NC(=O)Cc2c[nH]c3cc(F)ccc23)cc1F] | 10056 | ¹H NMR (400 MHz, DMSO-d₆) δ 10.98 (s, 1H), 10.12 (s, 1H), 7.62-7.53 (m, 2H), 7.29-7.23 (m, 2H), 7.15-7.06 (m, 2H), 6.90-6.81 (m, 1H), 3.78 (s, 3H), 3.68 (s, 2H). | 317 |
| 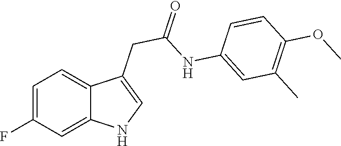 [SMILES: COc1ccc(NC(=O)Cc2c[nH]c3cc(F)ccc23)cc1C] | 10057 | ¹H NMR (400 MHz, DMSO-d₆) δ 10.96 (s, 1H), 9.85 (s, 1H), 7.62-7.54 (m, 1H), 7.41- 7.32 (m, 2H), 7.26-7.22 (m, 1H), 7.13- 7.10 (m, 1H), 6.90-6.81 (m, 2H), 3.73 (s, 3H), 3.66 (s, 2H), 2.10 (s, 3H). | 313 |
| 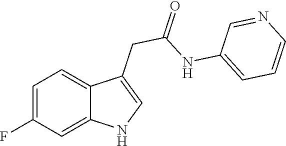 [SMILES: O=C(Cc1c[nH]c2cc(F)ccc12)Nc1cccnc1] | 10058 | ¹H NMR (400 MHz, DMSO-d₆) δ 11.00 (s, 1H), 10.32 (s, 1H), 8.74 (d, J = 2.3 Hz, 1H), 8.27-8.20 (m, 1H), 8.09-8.01 (m, 1H), 7.62-7.55 (m, 1H), 7.36-7.24 (m, 2H), 7.16-7.09 (m, 1H), 6.91-6.81 (m, 1H), 3.75 (s, 2H). | 270 |
| 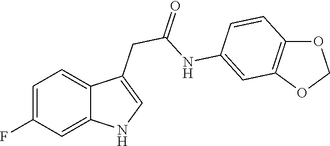 [SMILES: O=C(Cc1c[nH]c2cc(F)ccc12)Nc1ccc2c(c1)OCO2] | 10059 | ¹H NMR (400 MHz, DMSO-d₆) δ 10.97 (s, 1H), 10.01 (s, 1H), 7.60-7.54 (m, 1H), 7.32-7.30 (m, 1H), 7.25-7.23 (m, 1H), 7.14-7.10 (m, 1H), 6.98-6.94 (m, 1H), 6.88-6.81 (m, 2H), 5.96 (s, 2H), 3.67 (s, 2H). | 313 |
| 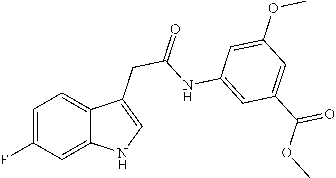 [SMILES: COC(=O)c1cc(NC(=O)Cc2c[nH]c3cc(F)ccc23)cc(OC)c1] | 10060 | ¹H NMR (400 MHz, DMSO-d₆) δ 11.00 (s, 1H), 10.33 (s, 1H), 7.83 (t, J = 1.6 Hz, 1H), 7.60-7.55 (m, 2H), 7.30-7.25 (m, 1H), 7.15-7.10 (m, 2H), 6.91-6.80 (m, 1H), 3.84 (s, 3H), 3.77 (s, 3H), 3.72 (s, 2H). | 357 |
| 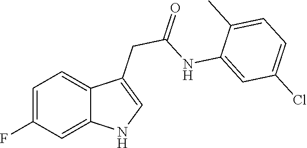 [SMILES: Cc1ccc(Cl)cc1NC(=O)Cc1c[nH]c2cc(F)ccc12] | 10061 | ¹H NMR (400 MHz, DMSO-d₆) δ 11.00 (s, 1H), 9.39 (s, 1H), 7.64-7.55 (m, 2H), 7.31- 7.25 (m, 1H), 7.23-7.18 (m, 1H), 7.16- 7.08 (m, 2H), 6.93-6.82 (m, 1H), 3.78 (s, 2H), 2.12 (s, 3H). | 317 |
| 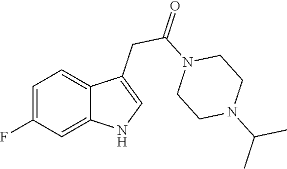 [SMILES: CC(C)N1CCN(C(=O)Cc2c[nH]c3cc(F)ccc23)CC1] | 10068 | ¹H NMR (400 MHz, DMSO-d₆) δ10.95 (s, 1H), 7.56-7.49 (m, 1H), 7.21-7.16 (m, 1H), 7.13-7.07 (m, 1H), 6.87-6.79 (m, 1H), 3.74 (s, 2H), 3.49-3.40 (m, 4H), 2.64- 2.56 (m, 1H), 2.34-2.24 (m, 4H), 0.90 (d, J = 6.5 Hz, 6H). | 304 |
| 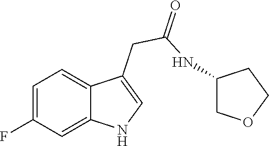 [SMILES: O=C(Cc1c[nH]c2cc(F)ccc12)N[C@@H]1CCOC1] | 10071 | ¹H NMR (400 MHz, DMSO-d₆) δ 10.91 (s, 1H), 8.29-8.17 (m, 1H), 7.58-7.48 (m, 1H), 7.19-7.08 (m, 2H), 6.89-6.79 (m, 1H), 4.26-4.17 (m, 1H), 3.81-3.63 (m, 3H), 3.47 (s, 2H), 3.45-3.41 (m, 1H), 2.12- 1.99 (m, 1H), 1.77-1.65 (m, 1H). | 263 |
| 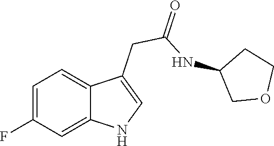 [SMILES: O=C(Cc1c[nH]c2cc(F)ccc12)N[C@H]1CCOC1] | 10070 | ¹H NMR (400 MHz, DMSO-d₆) δ 10.91 (s, 1H), 8.27-8.17 (m, 1H), 7.56-7.49 (m, 1H), 7.17-7.15 (m, 1H), 7.13-7.08 (m, 1H), 6.87-6.80 (m, 1H), 4.26-4.18 (m, 1H), 3.81-3.63 (m, 3H), 3.47 (s, 2H), 3.45- 3.42 (m, 1H), 2.11-2.01 (m, 1H), 1.75- 1.67 (m, 1H). | 263 |
| 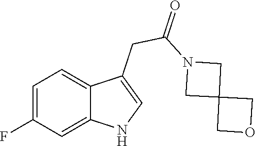 [SMILES: O=C(Cc1c[nH]c2cc(F)ccc12)N1CC2(COC2)C1] | 10069 | ¹H NMR (400 MHz, DMSO-d₆) δ 10.95 (s, 1H), 7.51-7.45 (m, 1H), 7.20-7.17 (m, 1H), 7.12-7.08 (m, 1H), 6.86-6.79 (m, 1H), 4.64 (s, 4H), 4.32 (s, 2H), 4.00 (s, 2H), 3.45 (s, 2H). | 275 |
| 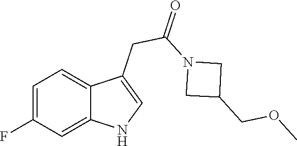 [SMILES: COCC1CN(C(=O)Cc2c[nH]c3cc(F)ccc23)C1] | 10075 | ¹H NMR (400 MHz, CDCl₃) δ 8.19 (s, 1H), 7.57-7.51 (m, 1H), 7.11-7.08 (m, 1H), 7.04-6.99 (m, 1H), 6.92-6.85 (m, 1H), 4.12 (s, 2H), 3.93-3.72 (m, 2H), 3.56 (s, 2H), 3.48 (d, J = 6.5 Hz, 2H), 3.35 (s, 3H), 2.85-2.72 (m, 1H). | 277 |
| 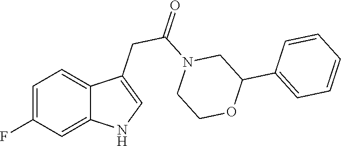 [SMILES: O=C(Cc1c[nH]c2cc(F)ccc12)N1CCOC(c2ccccc2)C1] | 10136 | ¹H NMR (400 MHz, DMSO-d₆) δ 11.11- 10.95 (m, 1H), 7.62-7.51 (m, 1H), 7.43- 7.21 (m, 5H), 7.20-7.09 (m, 2H), 6.91- 6.80 (m, 1H), 4.41-4.28 (m, 1H), 4.07- 3.89 (m, 3H), 3.84-3.74 (m, 1H), 3.50- 3.35 (m, 1H), 3.31-3.16 (m, 0.5H), 3.01- 2.92 (m, 0.5H), 2.84-2.74 (m, 0.5H), 2.68- 2.58 (m, 0.5H). | 339 |

### Example 3—Synthesis of (S)—N-(2-(6-fluoro-1H-indol-3-yl) ethyl) tetrahydrofuran-2-carboxamide (Compound #10023) (41-001 in Scheme 2)

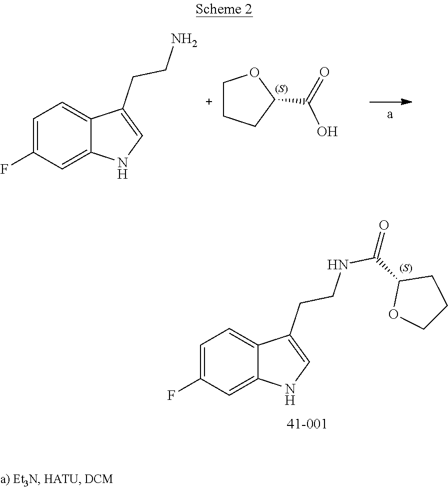

The solution of 2-(6-fluoro-1H-indol-3-yl)ethan-1-amine (100 mg, 0.47 mmol), (S)-tetrahydrofuran-2-carboxylic acid (76.0 mg, 0.65 mmol), HATU (354 mg, 0.93 mmol) and Et₃N (68.1 mg, 0.67 mmol) in DCM (8 mL) was stirred at room temperature for 16 hours. After which period, the mixture was diluted with dichloromethane (30 mL), which was then washed with HCl (15 mL, 1.0 N) and brine (20 mL), dried over sodium sulfate. After filtration and concentration, the residue was purified by flash column on silica gel to obtain desired product as colorless oil (68 mg, 52.3% yield).

MS (ESI+) m/z 277 (M+H)⁺.

¹H NMR (400 MHz, CDCl₃) δ 8.07 (s, 1H), 7.56-7.48 (m, 1H), 7.09-6.97 (m, 2H), 6.94-6.84 (m, 1H), 6.82-6.71 (m, 1H), 4.37-4.28 (m, 1H), 3.85-3.71 (m, 2H), 3.63-3.53 (m, 2H), 3.00-2.92 (m, 2H), 2.33- 2.19 (m, 1H), 2.08-1.96 (m, 1H), 1.92-1.68 (m, 2H).

### Example 4—Synthesis of Further Compounds Using the Procedure of Example 3

Using the above procedures, the following compounds were synthesized:

TABLE 2

| | Compound | | LC-MS |
|---|---|---|---|
| Structure | # | ¹H NMR | (M + H)⁺ |
| 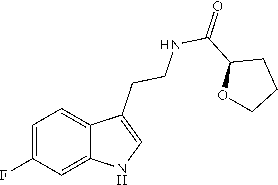 [SMILES: O=C(NCCc1c[nH]c2cc(F)ccc12)[C@H]1CCCO1] | 10013 | ¹H NMR (400 MHz, CDCl₃) δ 8.03 (s, 1H), 7.55-7.48 (m, 1H), 7.10-6.97 (m, 2H), 6.95- 6.85 (m, 1H), 6.81-6.71 (m, 1H), 4.39- 4.27 (m, 1H), 3.84-3.71 (m, 2H), 3.65-3.54 (m, 2H), 3.02-2.90 (m, 2H), 2.33-2.17 (m, 1H), 2.09-1.95 (m, 1H), 1.92-1.70 (m, 2H). | 277 |
| 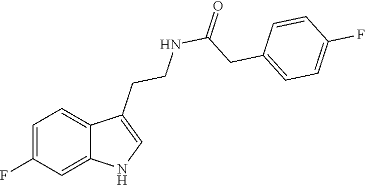 [SMILES: O=C(Cc1ccc(F)cc1)NCCc1c[nH]c2cc(F)ccc12] | 10012 | ¹H NMR (400 MHz, CDCl₃) δ 7.98 (s, 1H), 7.47-7.39 (m, 1H), 7.13-7.01 (m, 3H), 6.99- 6.91 (m, 2H), 6.91-6.84 (m, 1H), 6.82 (s, 1H), 5.37 (s, 1H), 3.60-3.50 (m, 2H), 3.47 (s, 2H), 2.94-2.83 (m, 2H). | 315 |
| 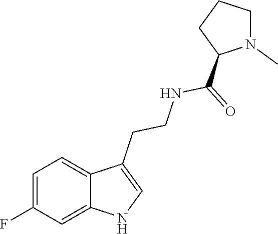 [SMILES: CN1CCC[C@@H]1C(=O)NCCc1c[nH]c2cc(F)ccc12] | 10028 | ¹H NMR (400 MHz, CDCl₃) δ 8.18 (s, 1H), 7.57-7.48 (m, 1H), 7.42 (s, 1H), 7.08-7.02 (m, 1H), 7.02-6.98 (m, 1H), 6.94-6.82 (m, 1H), 3.66-3.50 (m, 2H), 3.03-2.92 (m, 3H), 2.91-2.81 (m, 1H), 2.36-2.11 (m, 5H), 1.86- 1.55 (m, 3H). | 290 |
| 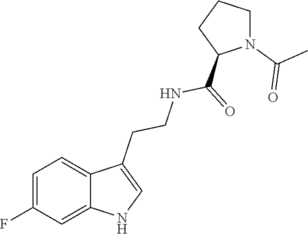 [SMILES: CC(=O)N1CCC[C@@H]1C(=O)NCCc1c[nH]c2cc(F)ccc12] | 10044 | ¹H NMR (400 MHz, CDCl₃) δ 8.16 (s, 1H), 7.53-7.46 (m, 1H), 7.17-6.96 (m, 3H), 6.93- 6.82 (m, 1H), 4.50 (d, J = 6.8 Hz, 0.8H), 4.27-4.18 (m, 0.2H), 3.69-3.30 (m, 4H), 3.01-2.85 (m, 2H), 2.47-2.35 (m, 1H), 2.23- 1.89 (m, 5H), 1.87-1.76 (m, 2H). | 318 |
| 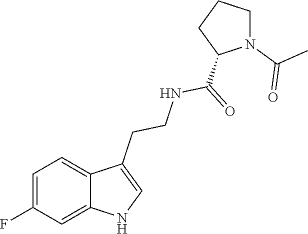 [SMILES: CC(=O)N1CCC[C@H]1C(=O)NCCc1c[nH]c2cc(F)ccc12] | 10045 | ¹H NMR (400 MHz, CDCl₃) δ 8.16 (s, 1H), 7.53-7.46 (m, 1H), 7.17-6.96 (m, 3H), 6.93- 6.82 (m, 1H), 4.50 (d, J = 6.8 Hz, 0.8H), 4.27-4.18 (m, 0.2H), 3.69-3.30 (m, 4H), 3.01-2.85 (m, 2H), 2.47-2.35 (m, 1H), 2.23- 1.89 (m, 5H), 1.87-1.76 (m, 2H). | 318 |
| 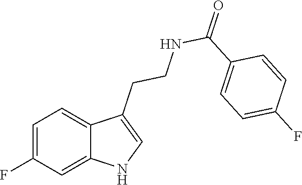 [SMILES: O=C(NCCc1c[nH]c2cc(F)ccc12)c1ccc(F)cc1] | 10039 | ¹H NMR (400 MHz, DMSO-d₆) δ 10.89 (s, 1H), 8.63 (t, J = 5.5 Hz, 1H), 7.96-7.86 (m, 2H), 7.60-7.51 (m, 1H), 7.35-7.24 (m, 2H), 7.21- 7.15 (m, 1H), 7.11 (dd, J = 10.2, 2.3 Hz, 1H), 6.88-6.80 (m, 1H), 3.57-3.47 (m, 2H), 2.93 (t, J = 7.4 Hz, 2H). | 301 |
| 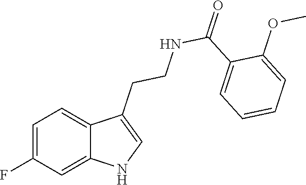 [SMILES: COc1ccccc1C(=O)NCCc1c[nH]c2cc(F)ccc12] | 10043 | ¹H NMR (400 MHz, DMSO-d₆) δ 10.94 (s, 1H), 8.22 (t, J = 5.4 Hz, 1H), 7.83-7.74 (m, 1H), 7.63-7.54 (m, 1H), 7.50-7.41 (m, 1H), 7.25- 7.20 (m, 1H), 7.16-7.08 (m, 2H), 7.05- 6.98 (m, 1H), 6.89-6.79 (m, 1H), 3.78 (s, 3H), 3.62-3.51 (m, 2H), 2.93 (t, J = 7.2 Hz, 2H). | 313 |
| 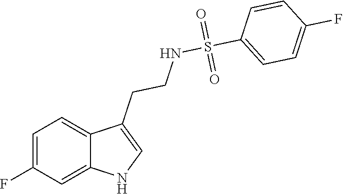 [SMILES: O=S(=O)(NCCc1c[nH]c2cc(F)ccc12)c1ccc(F)cc1] | 10046 | ¹H NMR (400 MHz, DMSO-d₆) δ 10.89 (s, 1H), 7.89-7.73 (m, 3H), 7.46-7.31 (m, 3H), 7.16- 7.04 (m, 2H), 6.86-6.75 (m, 1H), 3.05- 2.97 (m, 2H), 2.81-2.74 (m, 2H). | 337 |
| 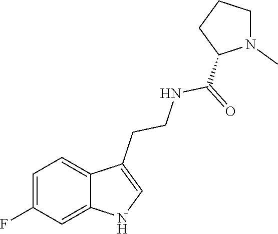 [SMILES: CN1CCC[C@H]1C(=O)NCCc1c[nH]c2cc(F)ccc12] | 10036 | ¹H NMR (400 MHz, CDCl₃) δ 8.12 (s, 1H), 7.56-7.49 (m, 1H), 7.47-7.34 (m, 1H), 7.07- 7.02 (m, 1H), 7.02-6.98 (m, 1H), 6.93- 6.83 (m, 1H), 3.67-3.51 (m, 2H), 3.05-2.92 (m, 3H), 2.91-2.82 (m, 1H), 2.36-2.12 (m, 5H), 1.85-1.54 (m, 3H). | 290 |
| 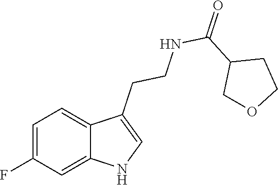 [SMILES: O=C(NCCc1c[nH]c2cc(F)ccc12)C1CCOC1] | 10063 | ¹H NMR (400 MHz, CDCl₃) δ 8.08 (s, 1H), 7.56-7.45 (m, 1H), 7.09-7.03 (m, 1H), 7.03- 6.99 (m, 1H), 6.93-6.86 (m, 1H), 5.69 (s, 1H), 3.93-3.71 (m, 4H), 3.64-3.53 (m, 2H), 3.02-2.90 (m, 2H), 2.85-2.74 (m, 1H), 2.15- 2.03 (m, 2H). | 277 |
| 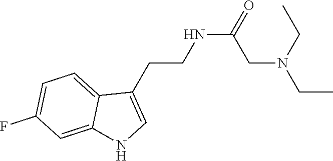 [SMILES: CCN(CC)CC(=O)NCCc1c[nH]c2cc(F)ccc12] | 10064 | ¹H NMR (400 MHz, CDCl₃) δ 8.14 (s, 1H), 7.57-7.41 (m, 2H), 7.11-6.98 (m, 2H), 6.95- 6.83 (m, 1H), 3.67-3.55 (m, 2H), 3.05- 2.91 (m, 4H), 2.44 (q, J = 7.1 Hz, 4H), 0.88 (t, J = 7.1 Hz, 6H). | 292 |

### Example 5—Synthesis of Ethyl (2-(6-fluoro-1H-indol-3-yl)acetyl)valinate (Compound #10079) (40-0608 in Scheme 3)

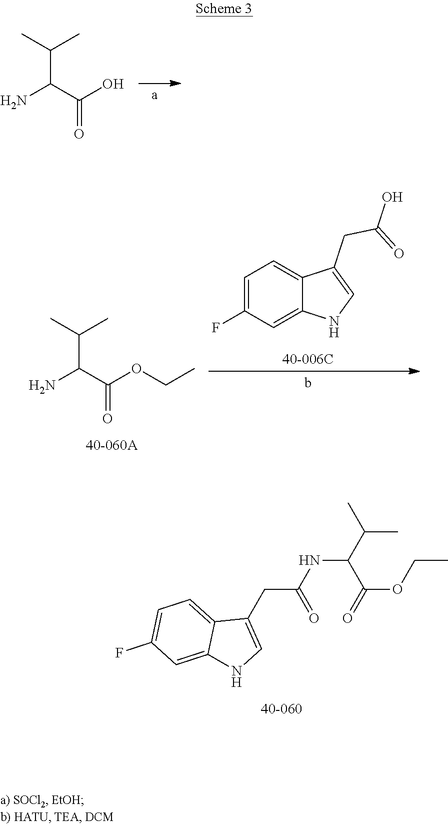

Ethyl valinate (40-060A)

To the solution of valine (500 mg, 4.27 mmol) in EtOH (5 mL) was added thionyl chloride (0.9 mL, 12.8 mmol) dropwise at 0° C. After addition, the mixture was heated to reflux for 6 hours. Then, the mixture was cooled down to room temperature and concentrated to get crude product for the next step without further purification.

MS (ESI+) m/z 146 (M+H)⁺.

### Ethyl (2-(6-fluoro-1H-indol-3-yl)acetyl)valinate (40-060B) (Compound #10079)

The solution of 2-(6-fluoro-1H-indol-3-yl) acetic acid (100 mg, 0.52 mmol), ethyl valinate (82.7 mg, 0.57 mmol), HATU (216.5 mg, 0.57 mmol) and Et₃N (172.9 mg, 1.71 mmol) in DCM (8 mL) was stirred at room temperature for 16 hours. After which period, the mixture was diluted with DCM (30 mL), which was washed with brine (20 mL), dried over sodium sulfate. After filtration and concentration, the residue was purified by flash column to obtain 72 mg colorless oil, which was further purified by Prep-HPLC and to get ethyl (2-(6-fluoro-1H-indol-3-yl)acetyl)valinate as white solid (36 mg, 21.7% yield).

MS (ESI+) m/z 321 (M+H)(.

¹H NMR (400 MHz, DMSO-d₆) δ 10.91 (s, 1H), 8.24 (d, J=8.2 Hz, 1H), 7.56-7.53 (2, 1H), 7.18 (s, 1H), 7.12-7.08 (m, 1H), 6.91-6.69 (m, 1H), 4.28-3.96 (m, 3H), 3.63-3.556 (i, 2H), 2.21-1.94 (m, 1H), 1.16 (t, J=7.1 Hz, 3H), 0.89-0.84 (m, 6H).

### Example 6—Synthesis of Further Compounds Using the Procedure of Example 5

Using the above procedures, the following compounds were synthesized:

TABLE 3

| | Compound | | LC-MS |
|---|---|---|---|
| Structure | # | ¹H NMR | (M + H)⁺ |
| 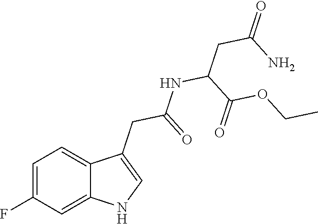 [SMILES: CCOC(=O)C(CC(N)=O)NC(=O)Cc1c[nH]c2cc(F)ccc12] | 10101 | ¹H NMR (400 MHz, DMSO-d₆) δ 10.92 (s, 1H), 8.29 (d, J = 7.8 Hz, 1H), 7.51 (dd, J = 8.7, 5.5 Hz, 1H), 7.36 (s, 1H), 7.19-7.16 (m, 1H), 7.10 (dd, J = 10.2, 2.3 Hz, 1H), 6.91 (s, 1H), 6.85-6.79 (m, 1H), 4.57-4.52 (m, 2H), 4.15-3.93 (m, 2H), 2.62-2.45 (m, 2H), 1.11 (t, J = 7.1 Hz, 3H). | 336 |
| 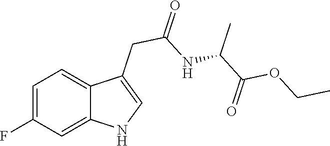 [SMILES: CCOC(=O)[C@@H](C)NC(=O)Cc1c[nH]c2cc(F)ccc12] | 10037 | ¹H NMR (400 MHz, CDCl₃) δ 8.26-8.15 (m, 1H), 7.55-7.46 (m, 1H), 7.18-7.13 (m, 1H), 7.11-7.04 (m, 1H), 6.96-6.88 (m, 1H), 6.27-6.11 (m, 1H), 4.63-4.52 (m, 1H), 4.12 (q, J = 7.1 Hz, 2H), 3.72 (s, 2H), 1.30 (d, J = 7.2 Hz, 3H), 1.21 (t, J = 7.1 Hz, 3H). | 293 |
| 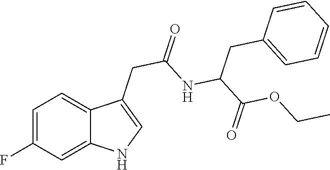 [SMILES: CCOC(=O)C(Cc1ccccc1)NC(=O)Cc1c[nH]c2cc(F)ccc12] | 10096 | ¹H NMR (400 MHz, DMSO-d₆) δ 10.90 (s, 1H), 8.43-8.32 (m, 1H), 7.42-7.33 (m, 1H), 7.29-7.13 (m, 5H), 7.13-7.03 (m, 2H), 6.83-6.72 (m, 1H), 4.50-4.40 (m, 1H), 4.09-3.96 (m, 2H), 3.49 (s, 2H), 3.06- 2.85 (m, 2H), 1.08 (t, J = 7.1 Hz, 3H). | 369 |
| 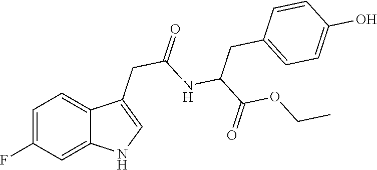 [SMILES: CCOC(=O)C(Cc1ccc(O)cc1)NC(=O)Cc1c[nH]c2cc(F)ccc12] | 10098 | ¹H NMR (400 MHz, DMSO-d₆) δ 10.90 (s, 1H), 9.23 (s, 1H), 8.35-8.23 (m, 1H), 7.38- 7.30 (m, 1H), 7.14-7.11 (m, 1H), 7.11- 7.06 (m, 1H), 6.99-6.93 (m, 2H), 6.82- 6.75 (m, 1H), 6.66-6.60 (m, 2H), 4.43- 4.30 (m, 1H), 4.08-3.93 (m, 2H), 3.50 (s, 2H), 2.94-2.74 (m, 2H), 1.09 (t, J = 7.1 Hz, 3H). | 385 |
| 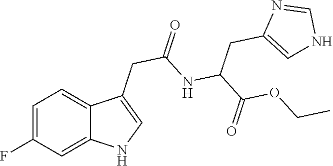 [SMILES: CCOC(=O)C(Cc1c[nH]cn1)NC(=O)Cc1c[nH]c2cc(F)ccc12] | 10104 | ¹H NMR (400 MHz, DMSO-d₆) δ 11.77 (s, 1H), 10.91 (s, 1H), 8.43-8.22 (m, 1H), 7.49 (d, J = 0.9 Hz, 1H), 7.45-7.36 (m, 1H), 7.15-7.12 (m, 1H), 7.11-7.07 (m, 1H), 6.86-6.72 (m, 2H), 4.52-4.39 (m, 1H), 4.08-3.95 (m, 2H), 3.51 (s, 2H), 2.98- 2.78 (m, 2H), 1.09 (t, J = 7.1 Hz, 3H). | 359 |
| 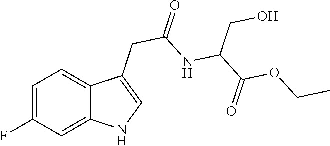 [SMILES: CCOC(=O)C(CO)NC(=O)Cc1c[nH]c2cc(F)ccc12] | 10105 | ¹H NMR (400 MHz, DMSO-d₆) δ 10.93 (s, 1H), 8.29-8.15 (m, 1H), 7.58-7.50 (m, 1H), 7.23-7.16 (m, 1H), 7.14-7.06 (m, 1H), 6.87-6.77 (m, 1H), 5.08-4.98 (m, 1H), 4.37-4.27 (m, 1H), 4.06 (q, J = 7.1 Hz, 2H), 3.77-3.52 (m, 4H), 1.19-1.10 (m, 3H). | 309 |
| 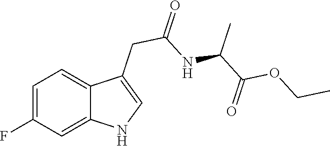 [SMILES: CCOC(=O)[C@H](C)NC(=O)Cc1c[nH]c2cc(F)ccc12] | 10038 | ¹H NMR (400 MHz, CDCl₃) δ 8.28-8.20 (m, 1H), 7.53-7.44 (m, 1H), 7.17-7.12 (m, 1H), 7.09-7.03 (m, 1H), 6.95-6.87 (m, 1H), 6.25-6.16 (m, 1H), 4.62-4.52 (m, 1H), 4.12 (q, J = 7.1 Hz, 2H), 3.72 (s, 2H), 1.30 (d, J = 7.2 Hz, 3H), 1.21 (t, J = 7.1 Hz, 3H). | 293 |
| 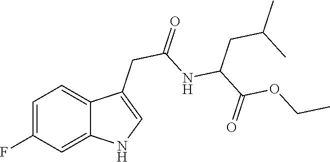 [SMILES: CCOC(=O)C(CC(C)C)NC(=O)Cc1c[nH]c2cc(F)ccc12] | 10093 | ¹H NMR (400 MHz, CDCl₃) δ 8.32 (s, 1H), 7.54-7.42 (m, 1H), 7.15-7.12 (m, 1H), 7.09-7.04 (m, 1H), 6.95-6.89 (m, 1H), 6.01-5.92 (m, 1H), 4.66-4.56 (m, 1H), 4.12 (q, J = 7.1 Hz, 2H), 3.75-3.71 (m, 2H), 1.57-1.42 (m, 2H), 1.40-1.32 (m, 1H), 1.22 (t, J = 7.1 Hz, 3H), 0.86 (d, J = 6.3 Hz, 3H), 0.82 (d, J = 6.4 Hz, 3H). | 335 |
| 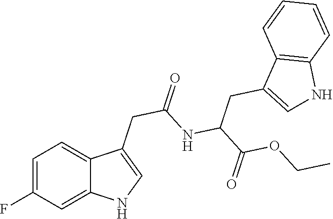 [SMILES: CCOC(=O)C(Cc1c[nH]c2ccccc12)NC(=O)Cc1c[nH]c2cc(F)ccc12] | 10112 | ¹H NMR (400 MHz, DMSO-d₆) δ 10.92- 10.84 (m, 2H), 8.37 (d, J = 7.6 Hz, 1H), 7.51- 7.44 (m, 1H), 7.40-7.30 (m, 2H), 7.16- 7.03 (m, 4H), 7.02-6.93 (m, 1H), 6.80- 6.71 (m, 1H), 4.57-4.45 (m, 1H), 4.00 (q, J = 7.1 Hz, 2H), 3.55-3.49 (m, 2H), 3.20- 2.99 (m, 2H), 1.05 (t, J = 7.1 Hz, 3H). | 408 |

### Example 7—Synthesis of 1-(azetidin-1-yl)-2-(6-fluoro-1-methyl-1H-indol-3-yl) ethan-1-one (Compound #10024) (40-022 in Scheme 4)

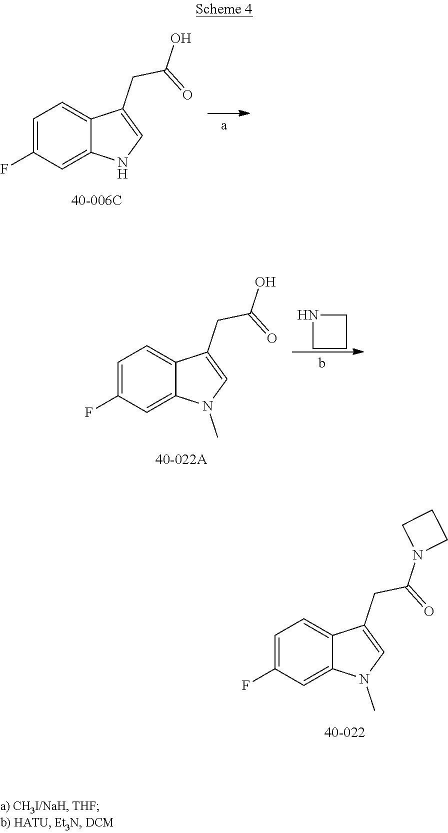

### 2-(6-fluoro-1-methyl-1H-indol-3-yl) acetic acid (40-022A)

A stirred suspension of sodium hydride (414.1 mg, 10.35 mmol) in THE (10 mL) was cooled down to 0° C. A solution of 2-(6-fluoro-1H-indol-3-yl) acetic acid (400 mg, 2.07 mmol) in THE (3 mL) was added to above system. Following stirring for 30 minutes at 0° C., a solution of iodomethane (0.43 mL, 6.83 mmol) in THE (2 mL) was added to the reaction mixture above dropwise. The resulting mixture was allowed to warm spontaneously to room temperature and stirred for 16 hours. Methanol (0.4 mL) was then cautiously added to the mixture, followed by water (7 mL) to quench the reaction. Ethyl acetate (10 mL) was added to the resulting clear yellow solution, after which the phases were separated. The aqueous phase was then acidified to pH 2 using 6M aqueous HCl and extracted with DCM (60 mL). The organic layer was dried over sodium sulfate and concentrated. Desired product was precipitated when hexane was then added and collected by filtration to get yellow solid (250 mg, 58.3% yield).

MS (ESI+) m/z 208 (M+H)⁺.

¹H NMR (400 MHz, CDCl₃) δ 7.50-7.47 (m, 1H), 7.01 (s, 1H), 6.98-6.95 (m, 1H), 6.91-6.85 (m, 1H), 3.76 (s, 2H), 3.71 (s, 3H).

### 1-(azetidin-1-yl)-2-(6-fluoro-1-methyl-1H-indol-3-yl) ethan-1-one (40-022) (Compound #10024)

The solution of 2-(6-fluoro-1-methyl-1H-indol-3-yl)acetic acid (100 mg, 0.48 mmol), azetidine (30.3 mg, 0.53 mmol), HATU (201.9 mmol, 0.53 mmol) and Et₃N (63.5 mg, 0.63 mmol) in DCM (8 mL) was stirred at room temperature for 16 hours. After which period, the mixture was diluted with DCM (10 mL), which was washed with brine (20 mL), dried over sodium sulfate. After filtration and concentration, the residue was purified by flash column to get desired product as white solid (50 mg, 42.1% yield).

MS (ESI+) m/z 247 (M+H)⁺.

¹H NMR (400 MHz, DMSO-d₆) δ 7.51 (dd, J=8.7, 5.5 Hz, 1H), 7.25 (dd, J=10.4, 2.3 Hz, 1H), 7.18 (s, 1H), 6.89-6.83 (m, 1H), 4.25-4.06 (m, 2H), 3.90-3.75 (m, 2H), 3.71 (s, 3H), 3.44 (s, 2H), 2.20-2.11 (m, 2H).

### Example 8—Synthesis of Bis(2-(6-fluoro-1H-indol-3-yl)ethyl)amine (Compound #10047) (40-034 in Scheme 5)

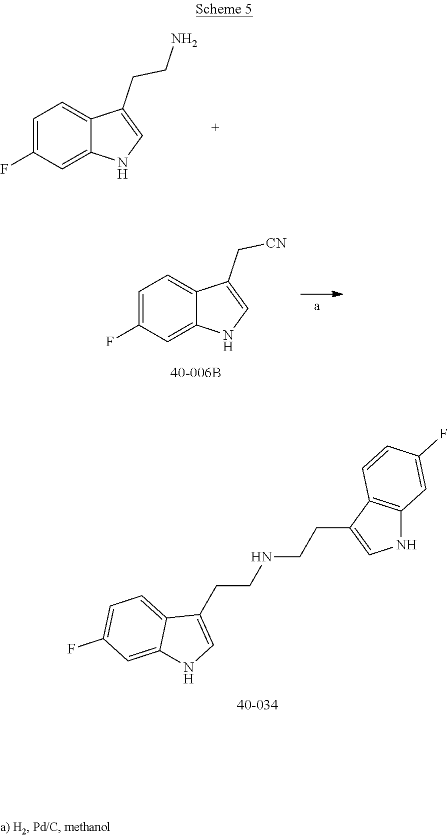 [SMILES: NCCc1c[nH]c2cc(F)ccc12 ; N#CCc1c[nH]c2cc(F)ccc12 ; Fc1ccc2c(CCNCCc3c[nH]c4cc(F)ccc34)c[nH]c2c1]

The solution of 2-(6-fluoro-1H-indol-3-yl)acetonitrile (100 mg, 0.57 mmol), 2-(6-fluoro-1H-indol-3-yl)ethan-1-amine (102.3 mg, 0.57 mmol) and Pd/C (30 mg) in methanol (10 mL) was stirred at room temperature under H₂ atmosphere for 16 hours. The mixture was filtered through celite and concentrated to get crude product, which was purified by Prep-HPLC to obtain title compound as colorless gel (12 mg, 6.2% yield).

MS (ESI+) m/z 340 (M+H)⁺.

¹H NMR (400 MHz, DMSO-d₆) δ 10.84 (s, 2H), 7.49-7.45 (m, 2H), 7.14-7.00 (m, 4H), 6.83-6.78 (m, 2H), 2.92-2.70 (m, 8H).

### Example 9—Synthesis of Ethyl 2-(6-fluoro-1H-indol-3-yl) acetate (Compound #10049) (40-046 in Scheme 6)

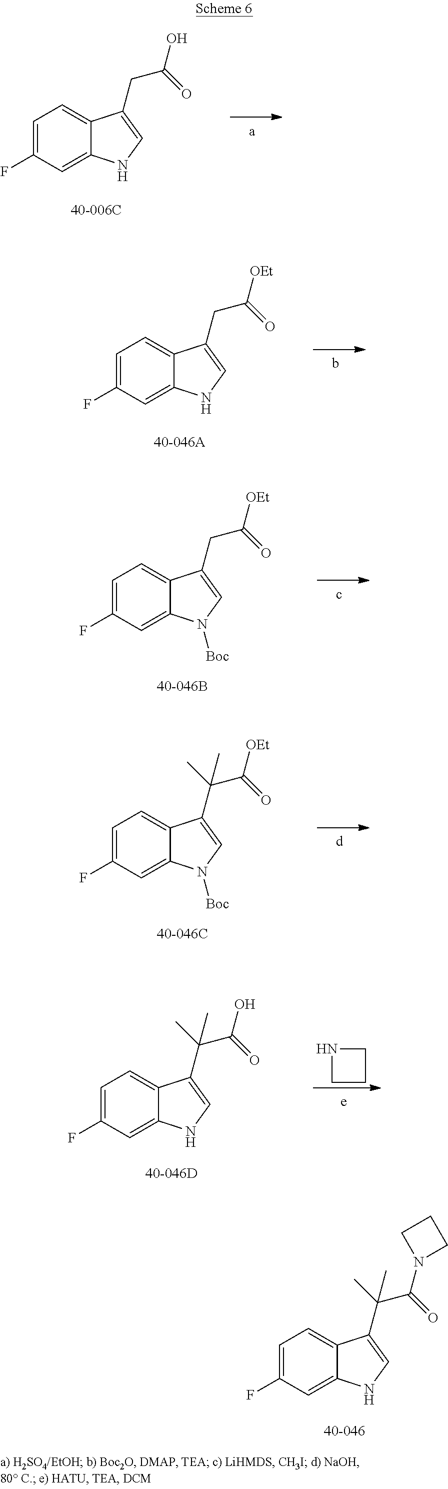

### Ethyl 2-(6-fluoro-1H-indol-3-yl) acetate (40-046A)

The solution of 2-(6-fluoro-1H-indol-3-yl) acetic acid (1 g, 5.18 mmol) and concentrated sulphuric acid (1.0 mL) in ethanol (24 mL) was refluxed for 3 hours. Then, the mixture was cooled down to 0° C. in an ice bath before neutralization with 2 N sodium hydroxide solution. The organics were evaporated under reduced pressure and the mixture was partitioned between dichloromethane (50 mL) and water (30 mL). The organic layer was separated and washed with brine (30 mL), dried over sodium sulfate. After filtration, the organic solution was concentrated under reduced pressure to give ethyl 2-(6-fluoro-1H-indol-3-yl) acetate (1.1 g, yellow oil, 96.0% yield), which was used for the next step without further purification.

MS (ESI+) m/z 222 (M+H)⁺.

### tert-Butyl 3-(2-ethoxy-2-oxoethyl)-6-fluoro-1H-indole-1-carboxylate (40-046B)

Ethyl 2-(6-fluoro-1H-indol-3-yl) acetate (750 mg, 3.39 mmol) was dissolved in THE (15 mL) and to the resulting solution was added Boc₂0 (998.6 mg, 4.58 mmol), Et₃N (0.5 mL, 4.07 mmol), and DMAP (41.4 mg, 0.34 mmol). The reaction was stirred at room temperature for 16 hours, and then was diluted with EtOAc (30 mL). The organic solution was washed with saturated NaHCO₃ (15 mL) and brine (15 mL), dried over Na₂SO₄ and concentrated to give crude product, which was purified by flash column to get titled compound as yellow oil (1 g, 91.8% yield).

¹H NMR (400 MHz, CDCl₃) δ 7.87-7.85 (m, 1H), 7.54 (s, 1H), 7.45 (dd, J=8.6, 5.3 Hz, 1H), 7.02-6.97 (m, 1H), 4.18 (q, J=7.1 Hz, 2H), 3.68-3.67 (m, 2H), 1.66 (s, 9H), 1.30-1.22 (m, 3H).

### tert-Butyl 3-(1-ethoxy-2-methyl-1-oxopropan-2-yl)-6-fluoro-1H-indole-1-carboxylate (40-046C)

The solution of tert-butyl 3-(2-ethoxy-2-oxoethyl)-6-fluoro-1H-indole-1-carboxylate (500 mg, 1.56 mmol) in dry THE (25 mL) under N₂ was cooled to −78° C. LiHMDS (15.6 mL, 15.56 mmol) was added dropwise and the mixture was continued to stir at −78° C. for 1 hour. Then, iodomethane (2.2 g, 15.56 mmol) in dry THE (4 mL) was added dropwise and stirred for 1 hour. The reaction was quenched with NH₄Cl (25 mL), extracted with ethyl acetate (100 mL), washed with brine (30 mL), dried over sodium sulfate. After filtration, the organic solution was concentrated to dryness, the residue was purified by flash column to get tert-butyl 3-(1-ethoxy-2-methyl-1-oxopropan-2-yl)-6-fluoro-1H-indole-1-carboxylate (260 mg, yellow oil, 47.8% yield)¹H NMR (400 MHz, CDCl₃) δ 7.86 (s, 1H), 7.46-7.43 (m, 2H), 6.97-6.92 (m, 1H), 4.12 (q, J=7.1 Hz, 2H), 1.72-1.60 (m, 15H), 1.13 (t, J=7.1 Hz, 3H).

And tert-butyl 3-(1-ethoxy-1-oxopropan-2-yl)-6-fluoro-1H-indole-1-carboxylate (33 mg, yellow oil, 6.3% yield).

### 2-(6-fluoro-1H-indol-3-yl)-2-methylpropanoic acid (40-046D)

The solution of tert-butyl 3-(1-ethoxy-2-methyl-1-oxopropan-2-yl)-6-fluoro-1H-indole-1-carboxylate (200 mg, 0.57 mmol) in EtOH (4 mL) was added sodium hydroxide (4 mL, 2 N). Then, the mixture was stirred at 80° C. for 5 hours. After cooling down to room temperature, the organic solvent was evaporated. The resulting aqueous solution was acidified by HCl (10 mL, 1N) to pH below 7. The precipitate was collected by filtration and washed with water (3 mL) to get white solid (100 mg, 79.0% yield).

MS (ESI+) m/z 222 (M+H)⁺.

¹H NMR (400 MHz, DMSO-d₆) δ 12.12 (s, 1H), 10.98 (s, 1H), 7.52-7.48 (m, 1H), 7.33-6.99 (m, 2H), 6.85-6.80 (m, 1H), 1.55 (s, 6H).

### 1-(azetidin-1-yl)-2-(6-fluoro-1H-indol-3-yl)-2-methylpropan-1-one (40-046) (Compound #10049)

The solution of 2-(6-fluoro-1H-indol-3-yl)-2-methylpropanoic acid (100 mg, 0.45 mmol), azetidine (28.4 mg, 0.50 mmol), HATU (189.1 mg, 0.50 mmol) and Et₃N (59.5 mg, 0.59 mmol) in DCM (8 mL) was stirred at room temperature for 16 hours. After which period, the mixture was diluted with DCM (30 mL), which was washed with brine (20 mL), dried over sodium sulfate. After filtration and concentration, the residue was purified by flash column to get desired product as white solid (60 mg, 51.0% yield).

MS (ESI+) m/z 261 (M+H)⁺.

¹H NMR (400 MHz, DMSO-d₆) δ 11.02 (s, 1H), 7.33-7.30 (m, 1H), 7.18 (d, J=2.4 Hz, 1H), 7.14 (dd, J=10.1, 2.3 Hz, 1H), 6.87-6.82 (m, 1H), 3.74 (t, J=7.5 Hz, 2H), 3.25 (t, J=7.4 Hz, 2H), 1.89-1.75 (m, 2H), 1.45 (s, 6H).

### Example 10—Synthesis of 1-(azetidin-1-yl)-2-(6-fluoro-1H-indol-3-yl) propan-1-one (Compound #10066) (40-057 in Scheme 7)

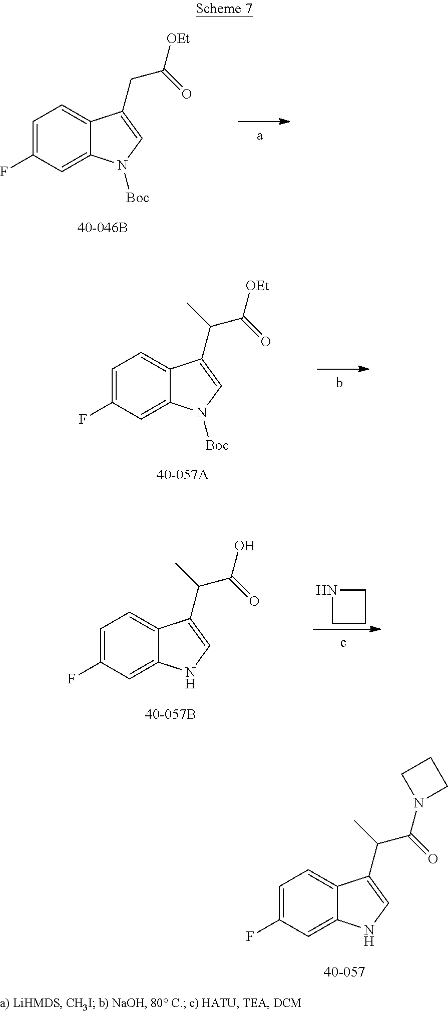

### tert-butyl 3-(1-ethoxy-1-oxopropan-2-yl)-6-fluoro-1H-indole-1-carboxylate (40-057A)

The solution of tert-butyl 3-(2-ethoxy-2-oxoethyl)-6-fluoro-1H-indole-1-carboxylate (400 mg, 1.24 mmol) in dry THE (8 mL) under N₂ was cooled to −78° C. LiHMDS (5.0 mL, 5.00 mmol) was added dropwise and the mixture was continued to stir at −78° C. for 1 hour. Then, iodomethane (709.7 mg, 5.00 mmol) in dry THE (0.5 mL) was added dropwise and stirred for 1 hour. The reaction was quenched with NH₄Cl (25 mL), extracted with ethyl acetate (50 mL), washed with brine (30 mL), dried over sodium sulfate, filtered and concentrated to purify by flash column to get tert-butyl 3-(1-ethoxy-1-oxopropan-2-yl)-6-fluoro-1H-indole-1-carboxylate as yellow oil (120 mg, 28.7% yield).

¹H NMR (400 MHz, CDCl₃) δ 7.79-7.77 (m, 1H), 7.52-7.37 (m, 2H), 6.94-6.89 (m, 1H), 4.19-4.00 (m, 2H), 3.93-3.75 (m, 1H), 1.59 (s, 9H), 1.52 (d, J=7.2 Hz, 3H), 1.14 (t, J=7.1 Hz, 3H).

### 1-(azetidin-1-yl)-2-(6-fluoro-1H-indol-3-yl) propan-1-one (40-057) (Compound #10066)

Compound 40-057 (Compound #10066) was prepared following the same procedures as that for synthesizing compound 40-046 (Compound #10049) in Example 9.

MS (ESI+) m/z 247 (M+H)⁺.

¹H NMR (400 MHz, DMSO-d₆) δ 10.96 (s, 1H), 7.59-7.55 (m, 1H), 7.17 (d, J=2.0 Hz, 1H), 7.11 (dd, J=10.2, 2.1 Hz, 1H), 6.93-6.71 (m, 1H), 4.22-4.16 (m, 1H), 3.97-3.65 (m, 4H), 2.16-2.13 (m, 2H), 1.34 (d, J=7.0 Hz, 3H).

### Example 11—Synthesis of 2-(6-fluoro-1H-indol-3-yl)-N-(pyrimidin-2-yl) acetamide (Compound #10078) (40-064 in Scheme 8)

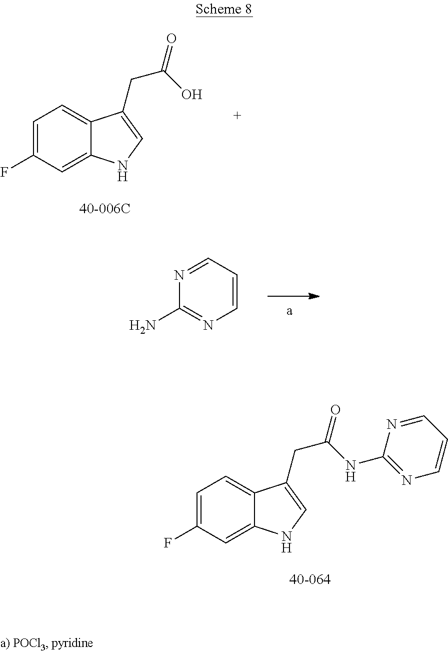 [SMILES: O=C(O)Cc1c[nH]c2cc(F)ccc12 ; Nc1ncccn1 ; O=C(Cc1c[nH]c2cc(F)ccc12)Nc1ncccn1]

The solution of 2-(6-fluoro-1H-indol-3-yl) acetic acid (150 mg, 0.78 mmol) and pyrimidin-2-amine (73.8 mg, 0.78 mmol) in pyridine (8 mL) was cooled to 0° C. POCl₃ (131.0 mg, 0.85 mmol) was added dropwise, and the resulting reaction mixture was stirred at room temperature for 2 hours. before cooling down to 0° C. again. Water was added slowly to quench the reaction. The mixture was then diluted with DCM (40 mL), which was then washed with saturated NaHCO₃ (40 mL) and brine (20 mL), dried over sodium sulfate. After filtration and concentration, the residue was purified by Prep-HPLC to get title compound as white solid (10 mg, 4.8% yield).

MS (ESI+) m/z 271 (M+H)⁺.

¹H NMR (400 MHz, DMSO-d₆) δ 10.97 (s, 1H), 10.65 (s, 1H), 8.63 (d, J=4.8 Hz, 2H), 7.58-7.55 (m, 1H), 7.25 (d, J=2.3 Hz, 1H), 7.19-7.08 (m, 2H), 6.87-6.82 (m, 1H), 3.87 (s, 2H).

### Example 12—Synthesis of 2-(6-fluoro-1H-indol-3-yl)-N-methyl-N-(1-(pyridin-4-yI)ethyl)acetamide (Compound #10100) (40-087 in Scheme 9)

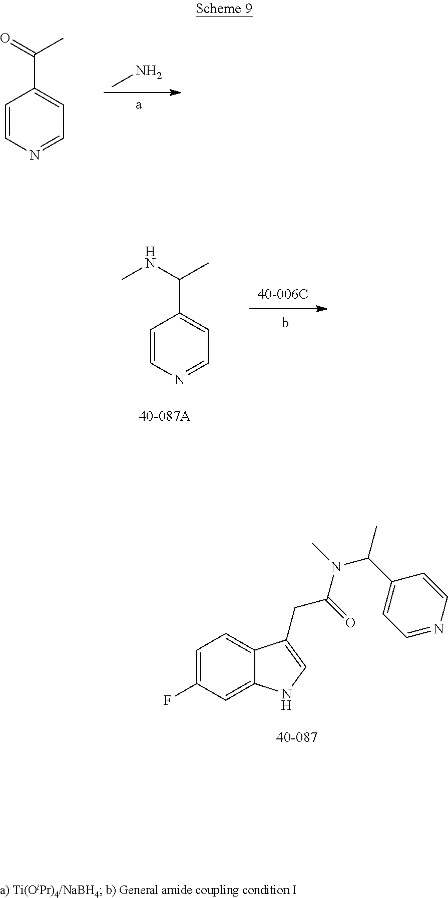

### N-methyl-1-(pyridin-4-yl) ethan-1-amine (40-087A)

The solution of 1-(pyridin-4-yl) ethan-1-one (1 g, 8.26 mmol) in methylamine (15 mL, 33% in ethanol) was cooled to 0° C., and titanium iso-propoxide (4.5 mL, 16.51 mmol) was added dropwise. The resulting mixture was stirred for 16 hours at room temperature before the addition NaBH₄ (624.6 mg, 16.51 mmol) at 0° C. The reaction was continued to stir at room temperature for another 2 hours. After which period, the reaction was quenched with iced water. The precipitate formed was filtered off through celite, the solid cake was washed with methanol for 3 times (20 mL). The combined filtrate was concentrated and then purified by flash column (DCM:MeOH: 5:1) to get title compound as colorless oil (730 mg, 64.9% yield).

MS (ESI+) m/z 137 (M+H)⁺.

¹H NMR (400 MHz, CDCl₃) δ 8.55 (dd, J=4.5, 1.6 Hz, 2H), 7.25 (dd, J=4.6, 1.5 Hz, 2H), 3.64 (q, J=6.6 Hz, 1H), 2.31 (s, 3H), 1.34 (d, J=6.6 Hz, 3H).

### 2-(6-fluoro-1H-indol-3-yl)-N-methyl-N-(1-(pyridin-4-yl)ethyl)acetamide (40-087) Compound #10100

The titled compound was prepared following general amide coupling condition I as white solid (60 mg, 37.2% yield).

MS (ESI+) m/z 312 (M+H)⁺.

¹H NMR (400 MHz, DMSO-d₆) δ 10.78 (s, 1H), 8.47 (dd, J=4.5, 1.6 Hz, 2H), 7.56 (dd, J=8.7, 5.5 Hz, 1H), 7.30-7.03 (m, 4H), 6.86-6.81 (m, 1H), 5.95-5.32 (m, 1H), 3.97-3.71 (m, 2H), 3.09 (s, 3H), 1.45 (d, J=7.1 Hz, 3H).

### Example 13—Synthesis of 2-(azetidin-1-yl)-1-(6-fluoro-1H-indol-3-yl) ethan-1-one Compound #10085 (42-075 in Scheme 10)

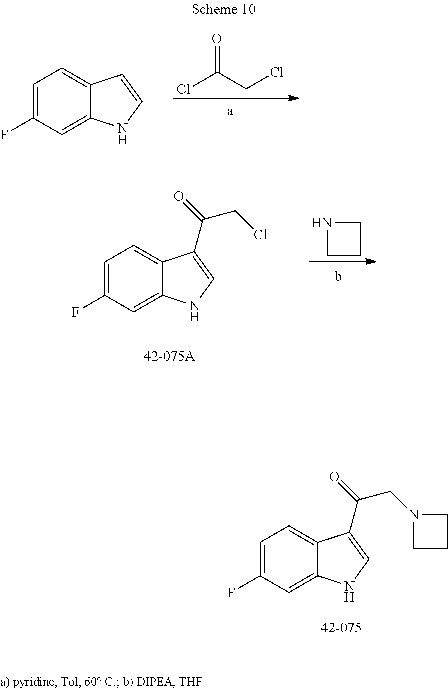

### 2-chloro-1-(6-fluoro-1H-indol-3-yl) ethan-1-one (42-075A)

To a stirred solution of 6-fluoro-1H-indole (1 g, 7.40 mmol) and pyridine (0.3 mL, 7.40 mmol) in toluene (12 mL) at 60° C. was added 2-chloroacetyl chloride (835.76 mg, 7.40 mmol) dropwise. After addition, the reaction mixture was stirred at 60° C. for 1 hour. After cooling to room temperature, H₂O (18 mL) and MeOH (4 mL) were added. The mixture was stirred at room temperature for additional 1 hour. The precipitate was filtered through a sintered glass funnel and washed with H₂O to get brown solid (300 mg, 19% yield).

¹H NMR (400 MHz, DMSO-d₆) δ 12.19 (s, 1H), 8.45 (d, J=3.1 Hz, 1H), 8.17-8.09 (m, 1H), 7.34-7.29 (m, 1H), 7.12-7.06 (m, 1H), 4.88 (s, 2H).

### 2-(azetidin-1-yl)-1-(6-fluoro-1H-indol-3-yl) ethan-1-one (42-075) (Compound #10085)

To a solution of 2-chloro-1-(6-fluoro-1H-indol-3-yl)ethan-1-one (200 mg, 0.94 mmol) and azetidine (80.94 mg, 1.42 mmol) in tetrahydrofuran (6 mL) was added DIPEA (0.3 mL, 1.89 mmol) dropwise and the resulting mixture was stirred at room temperature for 16 hours. The solvent was removed by vacuum (<25° C.) and the residue was precipitated out by treatment with ethyl acetate (10 mL). The precipitate was collected by filtration and was further purified by flash column to obtain title product as white solid (20.7 mg, 9% yield).

MS (ESI+) m/z 233 (M+H)⁺.

¹H NMR (400 MHz, DMSO-d₆) δ 11.98 (s, 1H), 8.37 (s, 1H), 8.15-8.09 (m, 1H), 7.28-7.23 (m, 1H), 7.07-7.00 (m, 1H), 3.76 (s, 2H), 3.31-3.28 (m, 4H), 2.10-2.00 (m, 2H).

### Example 14—Synthesis of Further Compounds Using the Procedure of Example 13

Using the above procedures, the following compounds were synthesized:

TABLE 4

| | Compound | | LC-MS |
|---|---|---|---|
| Structure | # | ¹H NMR | (M + H)⁺ |
| 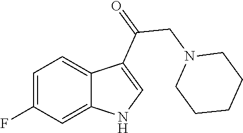 [SMILES: O=C(CN1CCCCC1)c1c[nH]c2cc(F)ccc12] | 10076 | ¹H NMR (400 MHz, CDCl₃) δ 8.70 (s, 1H), 8.43 − 8.40 (m, 1H), 8.39 − 8.33 (m, 1H), 7.13 − 7.01 (m, 2H), 3.57 (s, 2H), 2.55 (s, 4H), 1.66 − 1.60 (m, 4H), 1.50 − 1.43 (m, 2H). | 261 |

### Example 16—Synthesis of N-((6-fluoro-1H-indol-3-yl) methyl) aniline (Compound #10052) (41-040 in Scheme 12)

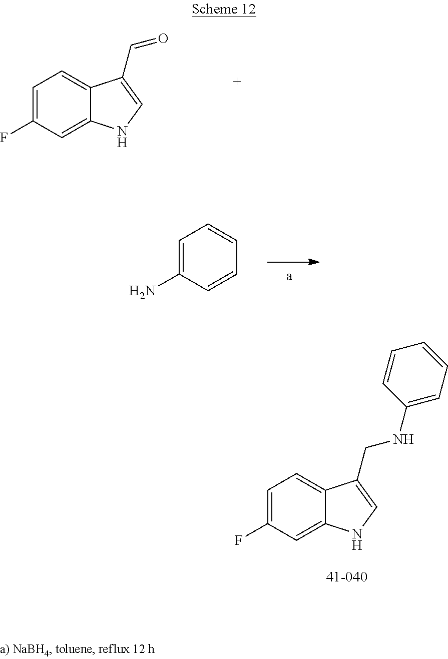 [SMILES: O=Cc1c[nH]c2cc(F)ccc12 ; Nc1ccccc1 ; Fc1ccc2c(CNc3ccccc3)c[nH]c2c1]

The solution of 6-fluoro-1H-indole-3-carbaldehyde (200 mg, 1.22 mmol) and aniline (114 mg, 1.22 mmol) in toluene (10 mL) was stirred in 50° C. for 12 hours. After cooling to 0° C., NaBH₄ (232 mg, 6.12 mmol) was added, stirred for 2 hours. The reaction was quenched with NH₄Cl (15 mL), extracted with ethyl acetate (20 mL), The separated organic phase was washed with brine (30 mL), dried over sodium sulfate. After filtration and concentration, the residue was purified by Prep-HPLC to get N-((6-fluoro-1H-indol-3-yl)methyl)aniline as yellow solid (21.5 mg, 7.8% yield).

MS (ESI+) m/z 271 (M+H)⁺.

¹H NMR (400 MHz, DMSO-d₆) δ 10.95 (s, 1H), 7.64-7.56 (m, 1H), 7.33-7.27 (m, 1H), 7.15-7.08 (m, 1H), 7.08-6.99 (m, 2H), 6.89-6.78 (m, 1H), 6.70-6.61 (m, 2H), 6.53-6.43 (m, 1H), 5.93-5.82 (m, 1H), 4.33 (d, J=5.5 Hz, 2H).

### Example 17—Synthesis of N-(2-(6-fluoro-1H-indol-3-yl)ethyl)-3-(methylsulfonamido)propenamide (Compound #10082) (41-053 in Scheme 13)

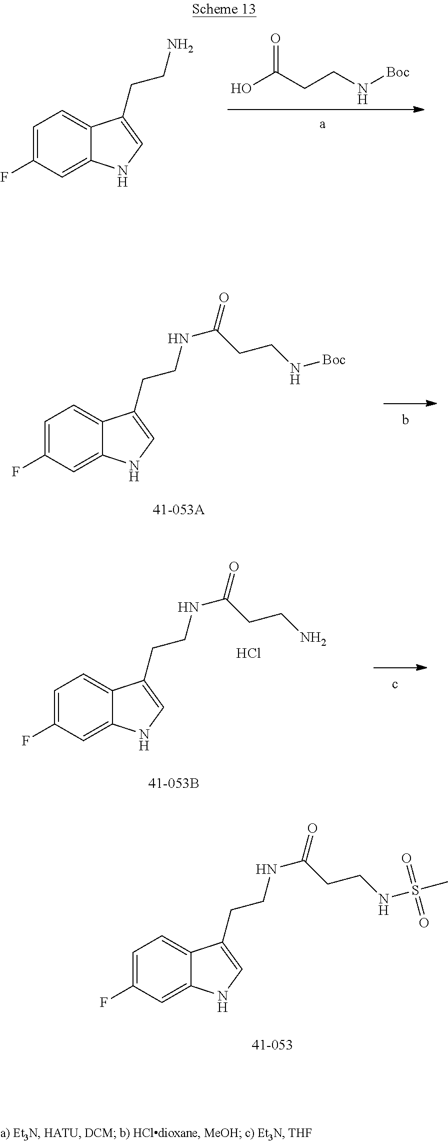 [SMILES: NCCc1c[nH]c2cc(F)ccc12 ; CC(C)(C)OC(=O)NCCC(=O)O ; CC(C)(C)OC(=O)NCCC(=O)NCCc1c[nH]c2cc(F)ccc12 ; NCCC(=O)NCCc1c[nH]c2cc(F)ccc12 ; Cl ; CS(=O)(=O)NCCC(=O)NCCc1c[nH]c2cc(F)ccc12]

### tert-butyl (3-((2-(6-fluoro-1H-indol-3-yl)ethyl)amino)-3-oxopropyl)carbamate (41-053A)

Following the general amide coupling procedures I, compound 41-053A was purified by flash column (elute with EA:PE=0:1 to 1:0) to get (458 mg, 93% yield) as white solid.

MS (ESI+) m/z 350 (M+H)⁺.

### 3-amino-N-(2-(6-fluoro-1H-indol-3-yl) ethyl) propanamide (41-053B)

The mixture of tert-butyl (3-((2-(6-fluoro-1H-indol-3-yl) ethyl) amino)-3-oxopropyl) carbamate (458 mg, 1.31 mmol) in HCl/dioxane (6N, 10 mL) and MeOH (10 mL) was stirred for 2 hours at room temperature. Then, the mixture was concentrated to get crude product for the next step without further purification.

MS (ESI+) m/z 250 (M+H)⁺.

### N-(2-(6-fluoro-1H-indol-3-yl)ethyl)-3-(methylsulfonamido)propanamide(41-053) (Compound #10082)

The mixture of 3-amino-N-(2-(6-fluoro-1H-indol-3-yl)ethyl)propanamide (178 mg, 0.71 mmol) and Et₃N (0.1 mL, 0.72 mmol) in THF (10 mL) was stirred at 0° C., MsCl (81.8 mg, 0.71 mmol) was added dropwise to above solution. After stirring for 12 hours at room temperature, DCM (15 mL) was added to dilute the reaction solution, which was then washed with H₂O (30 mL), then concentrated under reduced pressure. The resulting residue was purified by flash column (elute with EtOAc:PE=0:1 to 1:0) and Prep-HPLC to get the target product (95 mg, 40.6% yield) as a white solid.

MS (ESI+) m/z 328 (M+H)⁺.

¹H NMR (400 MHz, Acetone-d₆) δ 10.09 (s, 1H), 7.62-7.55 (m, 1H), 7.28 (s, 1H), 7.21-7.18 (m, 1H), 7.15-7.09 (m, 1H), 6.89-6.81 (m, 1H), 6.02 (s, 1H), 3.55-3.46 (m, 2H), 3.35 (q, J=6.4 Hz, 2H), 2.98-2.90 (m, 5H), 2.46 (t, J=6.6 Hz, 2H).

### Example 18—Synthesis of 2-(6-fluoro-1H-indol-3-yl)-N-(3-(2-methoxyacetamido)phenyl)acetamide (Compound #10080) (41-056 in Scheme 14)

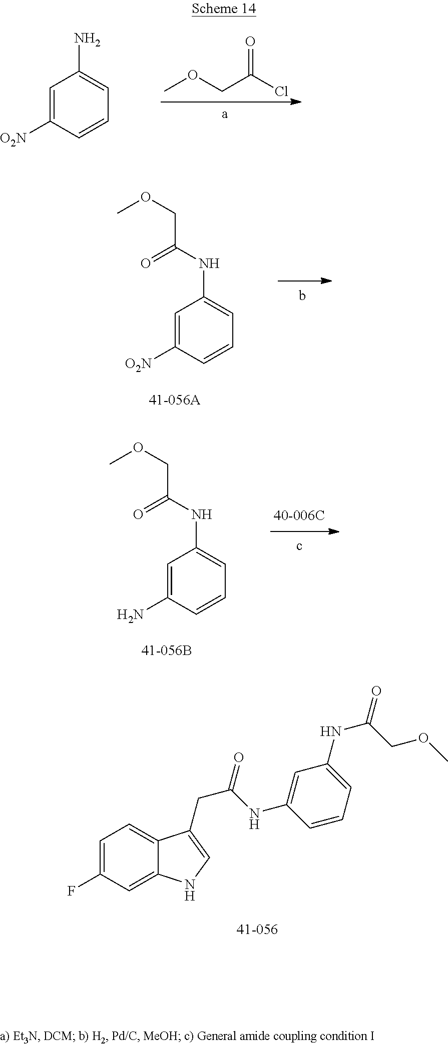

### 2-methoxy-N-(3-nitrophenyl)acetamide (41-056A)

The solution of 3-nitroaniline (200 mg, 1.44 mmol), 2-methoxyacetyl chloride (160 mg, 1.47 mmol) and Et₃N (0.2 mL, 1.44 mmol) in DCM (20 mL) was stirred at room temperature for 2 hours. The mixture was diluted with DCM (30 mL), washed with water (20 mL), dried over sodium sulfate, filtered and concentrated to purified by flash column (elute with EtOAc:PE=0:1 to 1:0) to get 2-methoxy-N-(3-nitrophenyl)acetamide (283 mg, 93% yield) as white solid.

MS (ESI+) m/z 211 (M+H)⁺.

### N-(3-aminophenyl)-2-methoxyacetamide(41-056B)

The solution of 2-methoxy-N-(3-nitrophenyl)acetamide (200 mg, 0.95 mmol) and Pd/C (60 mg) in MeOH (10 mL) was stirred at room temperature under H₂ for 12 hours. The mixture was filtered with celite and concentrated to get N-(3-aminophenyl)-2-methoxyacetamide (110 mg, 64% yield) as colorless oil.

MS (ESI+) m/z 181 (M+H)⁺.

### 2-(6-fluoro-1H-indol-3-yl)-N-(3-(2-methoxyacetamido)phenyl)acetamide(41-056) (Compound #10080)

Following general amide coupling condition I, the desired product was obtained as white solid.

MS (ESI+) m/z 356 (M+H)⁺.

¹H NMR (400 MHz, MeOH-d₄) δ 7.87-7.83 (m, 1H), 7.59-7.52 (m, 1H), 7.36-7.30 (m, 2H), 7.25 (d, J=8.0 Hz, 1H), 7.23-7.19 (m, 1H), 7.07-7.02 (m, 1H), 6.84-6.76 (m, 1H), 4.01 (s, 2H), 3.82-3.77 (m, 2H), 3.46 (s, 3H).

### Example 19—Synthesis of Further Compounds Using the Procedure of Example 18

Using the above procedures, the following compounds were synthesized:

TABLE 5

| | Compound | | LC-MS |
|---|---|---|---|
| Structure | # | ¹H NMR | (M + H)⁺ |
| 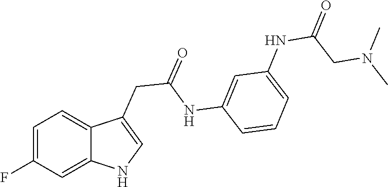 [SMILES: CN(C)CC(=O)Nc1cccc(NC(=O)Cc2c[nH]c3cc(F)ccc23)c1] | 10097 | ¹H NMR (400 MHz, MeOH-d₄) δ 10.97 (s, 1H), 10.10 (s, 1H), 9.66 (s, 1H), 8.00 − 7.93 (m, 1H), 7.63 − 7.54 (m, 1H), 7.36 − 7.31 (m, 1H), 7.28 − 7.23 (m, 2H), 7.21 − 7.15 (m, 1H), 7.12 (dd, J = 10.2, 2.3 Hz, 1H), 6.89 − 6.80 (m, 1H), 3.70 (s, 2H), 3.04 (s, 2H), 2.26 (s, 6H). | 369 |

### Example 20—Synthesis of N-(3-(2-(dimethylamino)ethoxy)phenyl)-2-(6-fluoro-1H-indol-3-yl)acetamide (Compound #10095) (41-065 in Scheme 15)

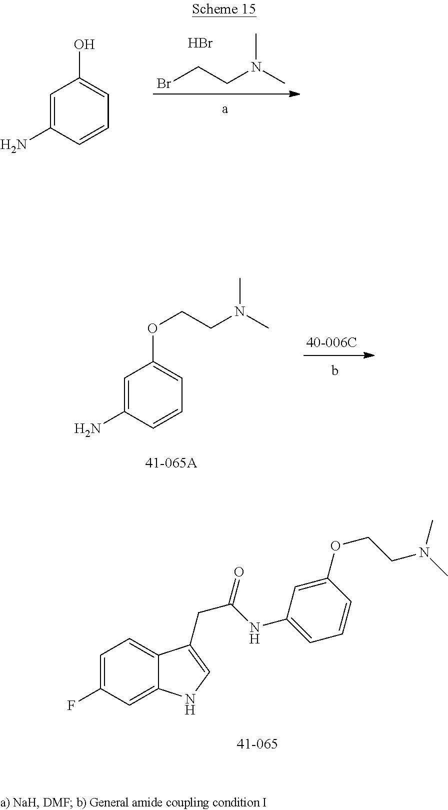

### 3-(2-(dimethylamino) ethoxy) aniline (41-065A)

Sodium hydride (183 mg, 4.58 mmol) was added to the solution of 3-aminophenol (500 mg, 4.58 mmol) in DMF (50 mL) at 0° C., and the mixture was stirred for 30 minutes. After then 2-bromo-N,N-dimethylethan-1-amine (1.07 g, 4.58 mmol) was added dropwise to above solution. The resulting mixture was stirred for 12 hours at room temperature before the addition of water (40 mL). The mixture was extracted with EtOAc (15 mL*3). The combined organic layers were washed with brine (30 mL), dried over sodium sulfate, filtered and concentrated. The residue was purified by flash column to get 3-(2-(dimethylamino)ethoxy)aniline (615 mg, 74.5% yield) as colorless oil.

MS (ESI+) m/z 181 (M+H)⁺.

### N-(3-(2-(dimethylamino)ethoxy)phenyl)-2-(6-fluoro-1H-indol-3-yl)acetamide(41-065) (Compound #10095)

Following general amide coupling condition I, the desired product was obtained as white solid.

MS (ESI+) m/z 356 (M+H)⁺.

¹H NMR (400 MHz, DMSO-d₆) δ 10.98 (s, 1H), 10.10-10.03 (m, 1H), 7.61-7.54 (m, 1H), 7.34-7.29 (m, 1H), 7.25 (d, J=2.3 Hz, 1H), 7.20-7.07 (m, 3H), 6.89-6.81 (m, 1H), 6.63-6.57 (m, 1H), 3.98 (t, J=5.8 Hz, 2H), 3.70 (s, 2H), 2.59 (t, J=5.8 Hz, 2H), 2.19 (s, 6H).

### Example 21—Synthesis of Further Compounds Using the Procedure of Example 20

Using the above procedures, the following compounds were synthesized:

TABLE 6

| | Compound | | LC-MS |
|---|---|---|---|
| Structure | # | ¹H NMR | (M + H)⁺ |
| 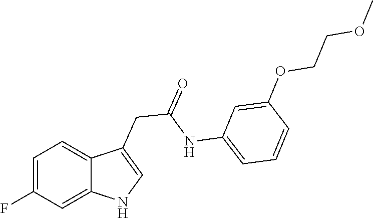 [SMILES: COCCOc1cccc(NC(=O)Cc2c[nH]c3cc(F)ccc23)c1] | 10094 | ¹H NMR (400 MHz, DMSO-d₆) δ 10.98 (s, 1H), 10.07 (s, 1H), 7.63 − 7.51 (m, 1H), 7.35 − 7.29 (m, 1H), 7.28 − 7.23 (m, 1H), 7.21 − 7.08 (m, 3H), 6.89 − 6.81 (m, 1H), 6.64 − 6.58 (m, 1H), 4.06 − 3.97 (m, 2H), 3.70 (s, 2H), 3.66 − 3.59 (m, 2H), 3.29 (s, 3H). | 343 |

### Example 22—Synthesis of N-(1,3-dimethylpiperidin-4-yl)-2-(6-fluoro-1H-indol-3-yl)acetamide (Compound #10099) (40-082 in Scheme 16)

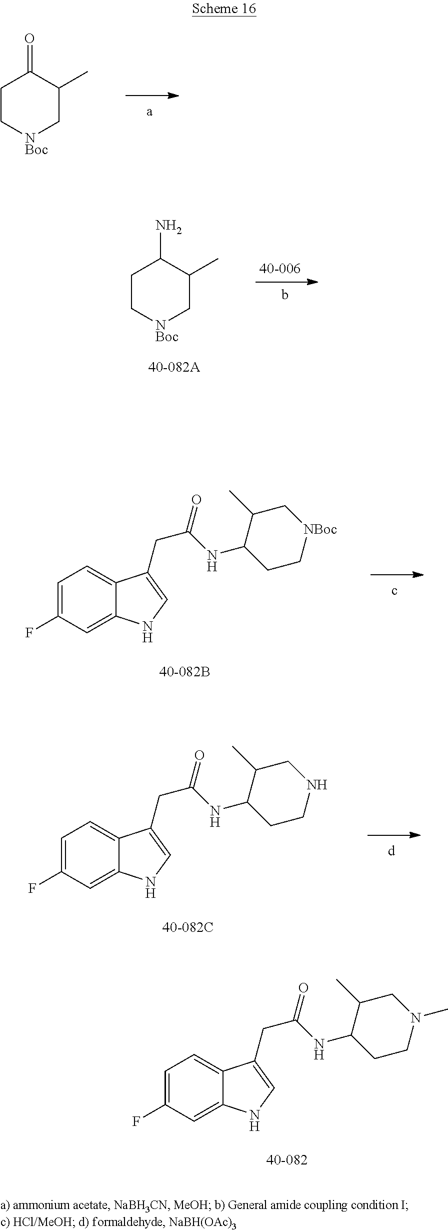 [SMILES: CC1CN(C(=O)OC(C)(C)C)CCC1=O ; CC1CN(C(=O)OC(C)(C)C)CCC1N ; CC1CN(C(=O)OC(C)(C)C)CCC1NC(=O)Cc1c[nH]c2cc(F)ccc12 ; CC1CNCCC1NC(=O)Cc1c[nH]c2cc(F)ccc12 ; CC1CN(C)CCC1NC(=O)Cc1c[nH]c2cc(F)ccc12]

### Tert-Butyl 4-amino-3-methylpiperidine-1-carboxylate (40-082A)

A suspension of tert-butyl 3-methyl-4-oxopiperidine-1-carboxylate (1 g, 4.69 mmol) and ammonium acetate (3 g, 38.92 mmol) in methanol (10 mL) was stirred for 18 hours at room temperature, then sodium cyanoborohydride (2 g, 31.68 mmol) was added and continued to stir at room temperature for 18 hours. The mixture was diluted with ethyl acetate (80 mL), washed with water (50 mL) and brine (30 mL), dried over sodium sulfate and concentrated to dryness. The residue was purified by flash column to obtain tert-butyl 4-amino-3-methylpiperidine-1-carboxylate (820 mg, 98.4% yield) as white solid.

MS (ESI+) m/z 215 (M+H)⁺.

### tert-butyl 4-(2-(6-fluoro-1H-indol-3-yl)acetamido)-3-methylpiperidine-1-carboxylate (40-082B)

Following general amide coupling condition I, the desired product was obtained.

MS (ESI+) m/z 334 (M+H)⁺.

### 2-(6-fluoro-1H-indol-3-yl)-N-(3-methylpiperidin-4-yl)acetamide (40-082C)

The solution of tert-butyl 4-(2-(6-fluoro-1H-indol-3-yl)acetamido)-3-methylpiperidine-1-carboxylate (370 mg, 0.95 mmol) in HCl (5 mL, 4.0 M in methanol) was stirred at room temperature for 3 hours. Then, the mixture was concentrated to get crude product for the next step without further purification.

MS (ESI+) m/z 290 (M+H)⁺.

### N-(1,3-dimethylpiperidin-4-yl)-2-(6-fluoro-1H-indol-3-yl)acetamide (40-082) (Compound #10099)

The solution of 2-(6-fluoro-1H-indol-3-yl)-N-(3-methylpiperidin-4-yl)acetamide (270 mg, 0.93 mmol), Et₃N (93.9 mg, 0.93 mmol) and formaldehyde (84.1 mg, 2.80 mmol) in DCM/MeOH (20 mL, 1:1) was stirred at room temperature for 2 hours. Then, AcOH (11.2 mg, 0.19 mmol) and NaBH(OAc)₃ (593.3 mg, 2.80 mmol) were added to above solution. The system was continued to stir at room temperature for 16 hours. After which period, saturated NaHCO₃ (30 mL) was added to the reaction. The resulting mixture was extracted with DCM (100 mL), dried over sodium sulfate, filtered and concentrated to dryness. The residue was purified by Prep-HPLC to obtain pure product (32 mg, 11.3% yield) as white solid.

MS (ESI+) m/z 304 (M+H)⁺.

¹H NMR (400 MHz, CDCl₃) δ 8.45 (s, 1H), 7.50-7.44 (m, 1H), 7.15-7.02 (m, 2H), 6.96-6.88 (m, 1H), 5.67 (d, J=8.1 Hz, 0.7H), 5.34 (d, J=8.8 Hz, 0.3H), 4.02-3.96 (m, 0.7H), 3.84-3.65 (m, 2H), 3.48-3.45 (m, 0.3H), 2.83-2.64 (m, 0.5H), 2.39-2.17 (m, 2H), 2.12-2.00 (m, 2.5H), 1.99-1.83 (m, 1.5H), 1.77 (s, 1H), 1.70-1.51 (m, 2H), 1.36-1.16 (m, 0.6H), 0.74 (d, J=6.6 Hz, 0.8H), 0.66 (d, J=6.6 Hz, 2.2H).

### Example 23—Synthesis of Compounds #10142 and 10119 (40-097 and 40-098 in Scheme 17, respectively)

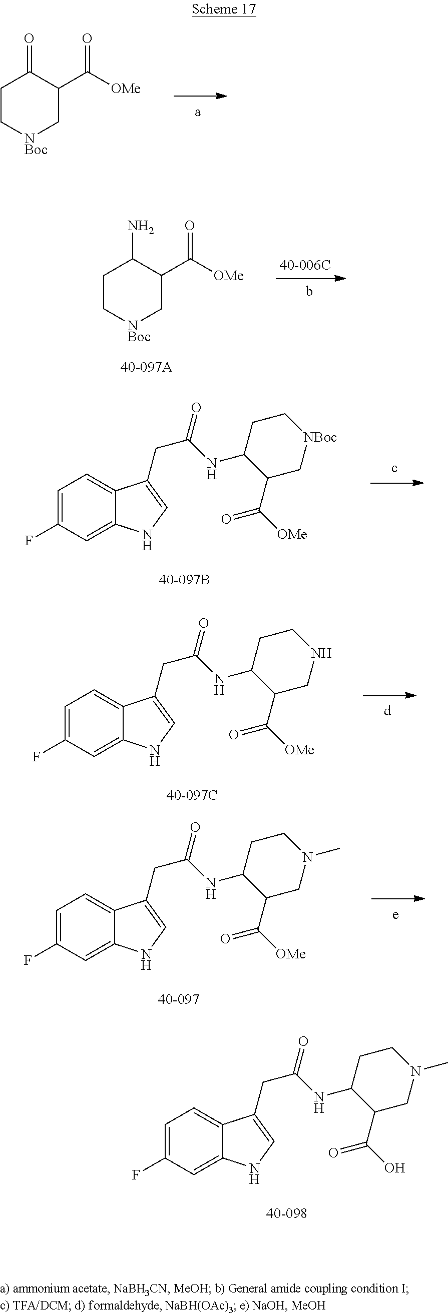 [SMILES: COC(=O)C1CN(C(=O)OC(C)(C)C)CCC1=O ; COC(=O)C1CN(C(=O)OC(C)(C)C)CCC1N ; COC(=O)C1CN(C(=O)OC(C)(C)C)CCC1NC(=O)Cc1c[nH]c2cc(F)ccc12 ; COC(=O)C1CNCCC1NC(=O)Cc1c[nH]c2cc(F)ccc12 ; COC(=O)C1CN(C)CCC1NC(=O)Cc1c[nH]c2cc(F)ccc12 ; CN1CCC(NC(=O)Cc2c[nH]c3cc(F)ccc23)C(C(=O)O)C1]

### 1-(tert-butyl) 3-methyl 4-aminopiperidine-1,3-dicarboxylate (40-097A)

Sodium cyanoborohydride (2 g, 31.83 mmol) was added to a solution of 1-(tert-butyl) 3-methyl 4-oxopiperidine-1,3-dicarboxylate (3.0 g, 11.66 mmol) and NH₄OAc (3.0 g, 38.92 mmol) in dry methanol (10 mL). The reaction mixture was stirred at room temperature for 16 hours. Additional NH₄OAc (2.2 g, 29.15 mmol) and Sodium cyanoborohydride (181.0 mg, 2.92 mmol) were added and the mixture was stirred for another 3 hours. After which period, the reaction was cooled to −10° C. and acidified to pH 2 with concentrated HCl. Solvent was removed under reduced pressure, and the solid residue was dissolved in water, and washed with ethyl ether. The pH of the aqueous phase was adjusted to 8-9 with solid KOH and the solution saturated with sodium chloride before being extracted repeatedly with ethyl acetate. The combined organic layers were dried over sodium sulfate and concentrated to afford the title compound (1.8 g, 60% yield).

MS (ESI+) m/z 259 (M+H)⁺.

### 1-(tert-butyl) 3-methyl 4-(2-(6-fluoro-1H-indol-3-yl)acetamido)piperidine-1,3-dicarboxylate (40-097B)

Following general amide coupling condition I, the desired product was obtained as yellow solid (1.8 g, 80% yield).

MS (ESI+) m/z 334 (M+H)⁺.

¹H NMR (400 MHz, CDCl₃) δ 8.61-8.53 (m, 1H), 7.47-7.37 (m, 1H), 7.14-7.04 (m, 2H), 6.95-6.83 (m, 1H), 6.47 (d, J=9.0 Hz, 0.5H), 5.68 (d, J=8.1 Hz, 0.5H), 4.33-3.75 (m, 3H), 3.70-3.64 (m, 2H), 3.55-3.38 (m, 4H), 3.08-2.95 (m, 1H), 2.72-2.64 (m, 0.5H), 2.28-2.16 (m, 0.5H), 1.98-1.91 (m, 0.5H), 1.82-1.65 (m, 1H), 1.59-1.51 (m, 0.5H), 1.40 (d, J=11.4 Hz, 9H).

### Methyl 4-(2-(6-fluoro-1H-indol-3-yl)acetamido)piperidine-3-carboxylate (40-097C)

The solution of 1-(tert-butyl) 3-methyl 4-(2-(6-fluoro-1H-indol-3-yl)acetamido)piperidine-1,3-dicarboxylate (1 g, 2.31 mmol) in TFA/DCM (5 mL, 1:1) was stirred at room temperature for 1 hour. Then, the mixture was concentrated, and the residue (1.2 g, crude) was used for the next step without further purification.

MS (ESI+) m/z 334 (M+H)⁺.

### Methyl 4-(2-(6-fluoro-1H-indol-3-yl) acetamido)-1-methylpiperidine-3-carboxylate (40-097) (Compound #10142)

The solution of methyl 4-(2-(6-fluoro-1H-indol-3-yl)acetamido)piperidine-3-carboxylate (311.1 mg, 0.93 mmol), Et₃N (93.3 mg, 0.93 mmol) and formaldehyde (84.1 mg, 2.80 mmol) in DCM/MeOH (20 mL, 1:1) was stirred at room temperature for 2 hours. Then, AcOH (5.6 mg, 0.1 mmol) and NaBH(OAc)₃ (593.3 mg, 2.80 mmol) were added and continued to stir at room temperature for 16 hours. After which period, saturated NaHCO₃ (30 mL) was added to the reaction. The resulting mixture was extracted with DCM (100 mL), dried over sodium sulfate, filtered and concentrated to dryness. The residue was purified by flash column to obtain methyl 4-(2-(6-fluoro-1H-indol-3-yl)acetamido)-1-methylpiperidine-3-carboxylate (200 mg, 61.7% yield) as white solid.

MS (ESI+) m/z 348 (M+H)⁺.

¹H NMR (400 MHz, CDCl₃) δ 8.18 (s, 1H), 7.45-7.12 (m, 1H), 7.15-7.03 (m, 2H), 6.94-6.83 (m, 1H), 6.61 (s, 1H), 4.15-4.05 (m, 1H), 3.67 (s, 2H), 3.45 (s, 3H), 3.05-2.85 (m, 1H), 2.72-2.51 (m, 2H), 2.30-2.12 (m, 4H), 2.10-2.04 (m, 1H), 1.92-1.75 (m, 1H), 1.65-1.58 (m, 1H).

### 4-(2-(6-fluoro-1H-indol-3-yl) acetamido)-1-methylpiperidine-3-carboxylic acid (40-098) (Compound #10119 and stereoisomers mixtures Compounds #10117 and 10118)

The solution of methyl 4-(2-(6-fluoro-1H-indol-3-yl)acetamido)-1-methylpiperidine-3-carboxylate (100 mg, 0.29 mmol) in methanol (5 mL) was added aqueous sodium hydroxide (5 mL, 1.0 N), then the mixture was stirred at room temperature for 16 hours. The solvent was removed under reduced pressure, HCl (1.0 N) was added to aqueous phase till pH below 7. The precipitate was collected by filtration and washed with water (3 mL*2), then dissolved in acetonitrile for Prep-HPLC to obtain (3S,4R)-4-(2-(6-fluoro-1H-indol-3-yl)acetamido)-1-methylpiperidine-3-carboxylic acid (3.6 mg, 3.8% yield), isomer (12.7 mg, 13.2% yield) and (3R,4R)-4-(2-(6-fluoro-1H-indol-3-yl)acetamido)-1-methylpiperidine-3-carboxylic acid (27 mg, 28.1% yield).

MS (ESI+) m/z 334 (M+H)⁺.

¹H NMR (400 MHz, DMSO-d₆) δ 10.92 (s, 1H), 8.19 (d, J=7.5 Hz, 1H), 7.49 (dd, J=8.7, 5.5 Hz, 1H), 7.23-7.01 (m, 2H), 6.85-6.79 (m, 1H), 4.10-3.95 (m, 1H), 3.50-3.41 (m, 4H), 3.15-2.60 (m, 6H), 1.96-1.82 (m, 1H), 1.68-1.56 (m, 1H).

¹H NMR (400 MHz, DMSO-d₆) δ 10.94 (s, 1H), 8.24-8.18 (m, 1H), 7.51-7.48 (m, 1H), 7.27-7.01 (m, 2H), 6.96-6.72 (m, 1H), 4.47 (s, 1H), 3.68-3.40 (m, 4H), 3.15-2.92 (m, 3H), 2.84-2.65 (m, 3H), 1.96- 1.72 (m, 2H).

¹H NMR (400 MHz, DMSO-d₆) δ 10.93 (s, 1H), 8.22-8.14 (m, 1H), 7.51-7.47 (m, 1H), 7.15-7.09 (m, 2H), 6.85-6.80 (m, 1H), 4.56 (s, 1H), 3.59-3.42 (m, 4H), 3.15-2.92 (m, 3H), 2.88-2.70 (m, 3H), 2.00- 1.72 (m, 2H).

### Example 24—Synthesis of methyl

### 4-(2-(6-fluoro-1H-indol-3-yl)acetamido)-1-isopropylpiperidine-3-carboxylate (Compound #10141) (40-099 in Scheme 18)

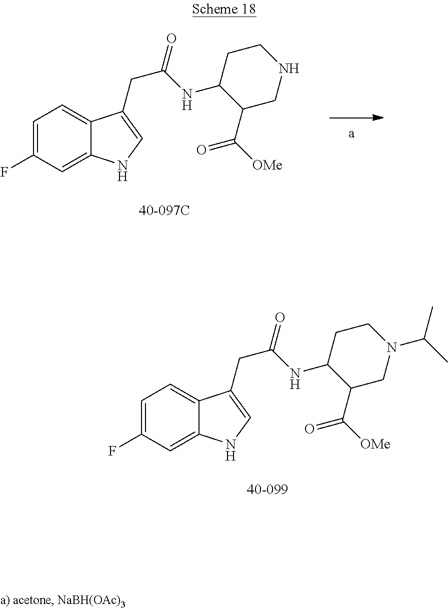 [SMILES: COC(=O)C1CNCCC1NC(=O)Cc1c[nH]c2cc(F)ccc12 ; COC(=O)C1CN(C(C)C)CCC1NC(=O)Cc1c[nH]c2cc(F)ccc12]

The solution of methyl 4-(2-(6-fluoro-1H-indol-3-yl)acetamido)piperidine-3-carboxylate (150 mg, 0.45 mmol), Et₃N (45.5 mg, 0.45 mmol) and acetone (78.4 mg, 1.35 mmol) in DCM/MeOH (20 mL, 1:1) was stirred at room temperature for 2 hours. Then, AcOH (2.7 mg, 0.05 mmol) and NaBH(OAc)₃ (286.1 mg, 1.35 mmol) were added and continued to stir at room temperature for 16 hours. After which period, saturated NaHCO₃ (30 mL) was added to the reaction. The resulting mixture was extracted with DCM (100 mL), dried over sodium sulfate, filtered and concentrated to dryness. The residue was purified by Prep-HPLC to obtain methyl 4-(2-(6-fluoro-1H-indol-3-yl)acetamido)-1-isopropylpiperidine-3-carboxylate (60 mg, 17.1% yield) as white solid.

MS (ESI+) m/z 376 (M+H)⁺.

¹H NMR (400 MHz, DMSO-d₆) δ 10.93 (s, 1H), 7.65 (d, J=7.8 Hz, 1H), 7.49-7.46 (m, 2H), 7.24-7.03 (m, 2H), 6.84-6.79 (m, 1H), 4.03 (s, 1H), 3.57-3.48 (m, 2H), 3.45 (s, 3H), 2.87-2.65 (m, 3H), 2.48-2.26 (m, 3H), 1.82-1.64 (m, 1H), 1.62-1.47 (m, 1H), 0.98-0.82 (m, 6H).

### Example 25—Synthesis of (S)—N-(1-acetylpiperidin-3-yl)-2-(6-fluoro-1H-indol-3-yl)acetamide (Compound #10073) (42-057 in Scheme 19)

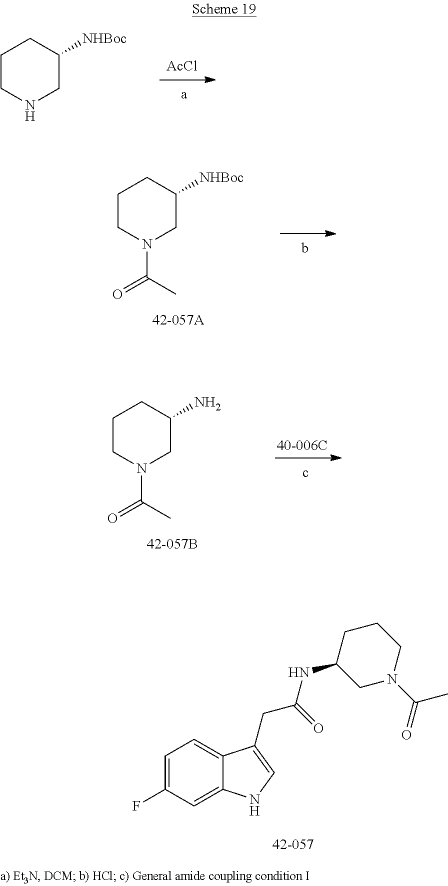

### tert-butyl (S)-(1-acetylpiperidin-3-yl) carbamate (42-057A)

To a stirred solution of tert-butyl (S)-piperidin-3-ylcarbamate (1 g, 4.99 mmol) and Et₃N (0.4 mL, 14.98 mmol) in DCM (14 mL) was added acetyl chloride (0.4 mL, 5.99 mmol) dropwise at 0° C. After addition, the mixture was stirred at room temperature for 1 hour. The resulting reaction mixture was poured into water (30 mL) and extracted with dichloromethane (40 mL*2). The combined organic layers were dried over sodium sulfate, filtered and concentrated to dryness. The residue was purified by flash column to obtain desired product as yellow oil (270 mg, 22% yield).

MS (ESI+) m/z 143 (M+H)⁺.

### (S)-1-(3-aminopiperidin-1-yl) ethan-1-one (42-057B)

The solution of tert-butyl (S)-(1-acetylpiperidin-3-yl) carbamate (270 mg, 1.11 mmol) in HCl (5 mL, 4.0 M in methanol) was stirred at room temperature for 4 hours. Then, the mixture was concentrated to get crude product for the next step without further purification (148 mg, 93% yield).

MS (ESI+) m/z 143 (M+H)⁺.

### (S)—N-(1-acetylpiperidin-3-yl)-2-(6-fluoro-1H-indol-3-yl)acetamide (42-057) Compound #10073

Following general amide coupling condition I, the desired product was obtained as white solid (43.3 mg, 26% yield).

MS (ESI+) m/z 318 (M+H)⁺.

¹H NMR (400 MHz, CDCl₃) δ 8.45 (d, J=18.7 Hz, 1H), 7.49-7.41 (m, 1H), 7.15-7.05 (m, 2H), 6.97-6.87 (m, 1H), 5.92-5.83 (m, 0.5H), 5.72-5.63 (m, 0.5H), 3.97-3.87 (m, 1H), 3.77-3.62 (m, 3H), 3.60-3.27 (m, 2H), 3.18-3.08 (m, 1H), 1.96-1.82 (m, 2H), 1.80-1.65 (m, 3H), 1.47-1.33 (m, 2H).

### Example 26—Synthesis of Further Compounds Using the Procedure of Example 25

Using the above procedures, the following compounds were synthesized:

TABLE 7

| | Compound | | LC-MS |
|---|---|---|---|
| Structure | # | ¹H NMR | (M + H)⁺ |
| 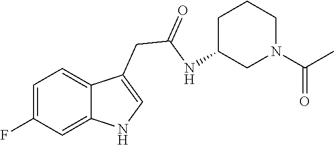 [SMILES: CC(=O)N1CCC[C@@H](NC(=O)Cc2c[nH]c3cc(F)ccc23)C1] | 10074 | ¹H NMR (400 MHz, CDCl₃) δ 8.56 (d, J = 23.2 Hz, 1H), 7.50 − 7.42 (m, 1H), 7.18 − 7.04 (m, 2H), 6.98 − 6.85 (m, 1H), 5.95 − 5.83 (m, 0.5H), 5.74 − 5.66 (m, 0.5H), 3.97 − 3.86 (m, 1H), 3.78 − 3.54 (m, 3H), 3.53 − 3.34 (m, 1H), 3.33 − 3.08 (m, 2H), 1.88 (s, 2H), 1.80 − 1.62 (m, 3H), 1.48 − 1.31 (m, 2H). | 318 |
| 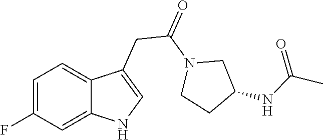 [SMILES: CC(=O)N[C@@H]1CCN(C(=O)Cc2c[nH]c3cc(F)ccc23)C1] | 10091 | ¹H NMR (400 MHz, CDCl₃) δ 8.47 − 8.31 (m, 1H), 7.60 − 7.51 (m, 1H), 7.09 − 6.99 (m, 2H), 6.93 − 6.83 (m, 1H), 5.87 − 5.79 (m, 0.5H), 5.78 − 5.67 (m, 0.5H), 4.46 − 4.35 (m, 1H), 3.77 − 3.65 (m, 3H), 3.63 − 3.52 (m, 2H), 3.42 − 3.33 (m, 1H), 2.26 − 1.99 (m, 1H), 1.97 − 1.86 (m, 3H), 1.82 − 1.69 (m, 1H). | 304 |

### Example 27—Synthesis of 2-(6-fluoro-1H-indol-3-yl)-1-(4-isobutoxypiperidin-1-yl) ethan-1-one (Compound #10111) (42-086 in Scheme 20)

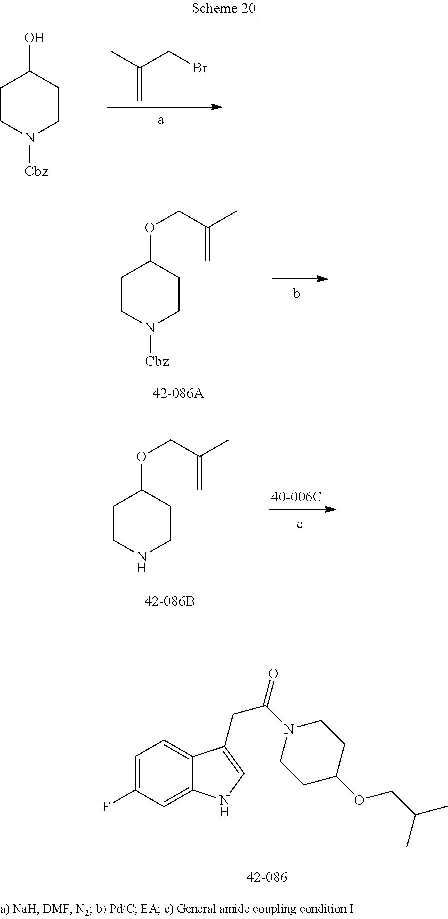

### Benzyl 4-((2-methylallyl) oxy)piperidine-1-carboxylate (42-086A)

To a stirred suspension of NaH (306.02 mg, 12.75 mmol) in DMF (6 mL) at 0° C. under nitrogen, benzyl 4-hydroxypiperidine-1-carboxylate (1 g, 4.25 mmol) in DMF (6 mL) was added dropwise. After stirring at 0° C. for 15 minutes, 3-bromo-2-methylprop-1-ene (0.86 mL, 8.5 mmol) was added to above solution dropwise, and the resulting solution was slowly warmed to room temperature and continued to stir for another 2 hours. The reaction was quenched at 0° C. by addition of aqueous saturated NaHCO₃ (80 mL), and extracted with EtOAc (60 mL). The separated organic phase was washed with brine (30 mL), dried over sodium sulfate and concentrated to dryness. The residue was purified by flash column to obtain title compound (1.2 g, 98% yield).

¹H NMR (400 MHz, CDCl₃) δ 7.39-7.28 (m, 5H), 5.17-5.10 (m, 2H), 4.99-4.95 (m, 1H), 4.91-4.86 (m, 1H), 3.91 (s, 2H), 3.85-3.75 (m, 2H), 3.55-3.46 (m, 1H), 3.29-3.19 (m, 2H), 1.88-1.78 (m, 2H), 1.74 (s, 3H), 1.66-1.50 (m, 2H).

### 4-isobutoxypiperidine (42-086B)

To a solution of benzyl 4-((2-methylallyl)oxy)piperidine-1-carboxylate (1.2 g, 4.35 mmol) in EtOAc (16 mL) was added Pd/C (120 mg, 1.13 mmol). The atmosphere was replaced with hydrogen and the mixture was stirred for 16 hours. The reaction was filtered through celite and the filter cake was then washed with EtOAc (30 mL). The filtrate was concentrated to give title compound as yellow oil (523 mg, 76% yield).

MS (ESI+) m/z 158 (M+H)⁺.

### 2-(6-fluoro-1H-indol-3-yl)-1-(4-isobutoxypiperidin-1-yl) ethan-1-one (42-086) (Compound #10111)

Following general amide coupling condition I, the desired product was obtained as white solid (40 mg, 23% yield).

MS (ESI+) m/z 333 (M+H)⁺.

¹H NMR (400 MHz, DMSO-d₆) δ 10.95 (s, 1H), 7.57-7.51 (m, 1H), 7.21-7.18 (m, 1H), 7.13-7.08 (m, 1H), 6.87-6.79 (m, 1H), 3.87-3.77 (m, 1H), 3.76-3.66 (m, 3H), 3.44-3.35 (m, 1H), 3.29-3.17 (m, 1H), 3.16-3.04 (m, 3H), 1.76-1.65 (m, 2H), 1.64-1.56 (m, 1H), 1.31-1.13 (m, 2H), 0.87-0.79 (m, 6H).

### Example 28—Synthesis of (S)—N-(1-(2-(6-fluoro-1H-indol-3-yl) acetyl) piperidin-3-yl) acetamide (Compound #10139) (42-108 in Scheme 21)

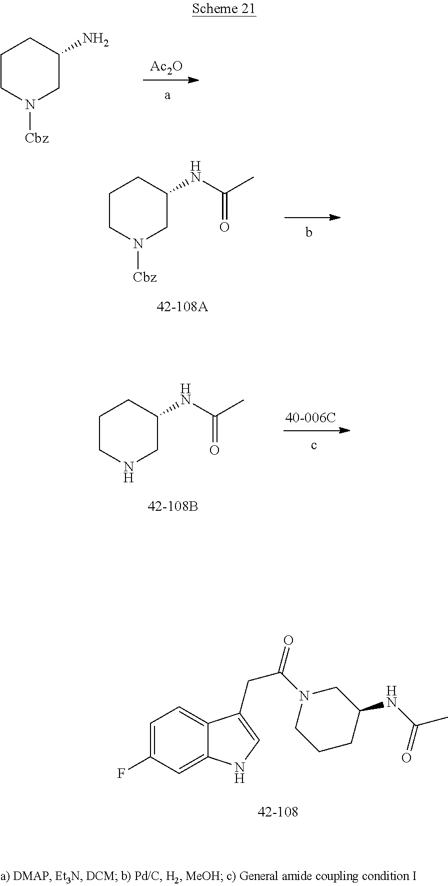

### benzyl (S)-3-acetamidopiperidine-1-carboxylate (42-108A)

Acetic anhydride (335.5 mg, 3.29 mmol) was added to the solution of benzyl (S)-3-aminopiperidine-1-carboxylate (700 mg, 2.99 mmol), Et₃N (435.5 mg, 4.48, mmol) and DMAP (54.8 mg, 0.45 mmol) in DCM (15 mL) at 0° C. After addition, the reaction was slowly warmed to room temperature and stirred for 4 hours. Then, the mixture was diluted with DCM (30 mL), washed with water (50 mL), dried over sodium sulfate, filtered and concentrated to dryness. The crude product was obtained as yellow solid (800 mg, 97% yield), Which was used to next step directly.

MS (ESI+) m/z 277 (M+H)⁺.

### (S)—N-(piperidin-3-yl) acetamide (42-108B)

The solution of benzyl (S)-3-acetamidopiperidine-1-carboxylate (0.8 g, 2.89 mmol) in EtOAc (8 mL) was added Pd/C (80 mg). The atmosphere was replaced with hydrogen and the mixture was stirred for 16 hours. The reaction was filtered, and filter cake was then washed with EtOAc (30 mL). The filtrate was concentrated to give title compound as give yellow oil (420 mg, 99% yield).

¹H NMR (400 MHz, DMSO-d₆) δ 7.86-7.69 (m, 1H), 3.72-3.60 (m, 1H), 3.30 (s, 1H), 3.04-2.94 (m, 1H), 2.91-2.81 (m, 1H), 2.57-2.45 (m, 1H), 2.40-2.29 (m, 1H), 1.93-1.82 (m, 4H), 1.76-1.63 (m, 1H), 1.54-1.28 (m, 2H).

### (S)—N-(1-(2-(6-fluoro-1H-indol-3-yl) acetyl) piperidin-3-yl) acetamide (42-108) (Compound #10139)

Following general amide coupling condition I, the desired product was obtained as white solid (50 mg, 30% yield).

MS (ESI+) m/z 318 (M+H)⁺.

¹H NMR (400 MHz, CDCl₃) δ 8.27 (s, 1H), 7.69-7.53 (m, 1H), 7.14-6.97 (m, 2H), 6.96-6.84 (m, 1H), 5.54-5.43 (m, 0.5H), 5.33-5.18 (m, 0.5H), 5.32-5.20 (m, 1H), 3.98-3.73 (m, 4H), 3.64 (s, 1H), 3.46 (s, 1H), 3.34-3.24 (m, 1H), 1.86-1.68 (m, 4H), 1.61-1.46 (m, 1H), 1.41-1.21 (m, 1H).

### Example 29—Synthesis of Further Compounds Using the Procedure of Example 28

Using the above procedures, the following compounds were synthesized:

TABLE 8

| | Compound | | LC-MS |
|---|---|---|---|
| Structure | # | ¹H NMR | (M + H)⁺ |
| 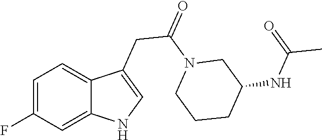 [SMILES: CC(=O)N[C@@H]1CCCN(C(=O)Cc2c[nH]c3cc(F)ccc23)C1] | 10137 | ¹H NMR (400 MHz, CDCl₃) δ 8.25 (s, 1H), 7.63 − 7.59 (m, 1H), 7.17 − 6.98 (m, 2H), 6.95 − 6.85 (m, 1H), 5.56 − 5.42 (m, 0.5H), 5.32 − 5.17 (m, 0.5H), 3.98 − 3.76 (m, 4H), 3.73 − 3.57 (m, 1H), 3.52 − 3.39 (m, 1H), 3.36 − 3.23 (m, 1H), 1.88 − 1.47 (m, 6H), 1.34 (s, 1H). | 318 |

### Example 30—Synthesis of (S)-1-(2-(6-fluoro-1H-indol-3-yl)acetyl)piperidine-3-carboxylic acid (Compound #10133) (42-117 in Scheme 22)

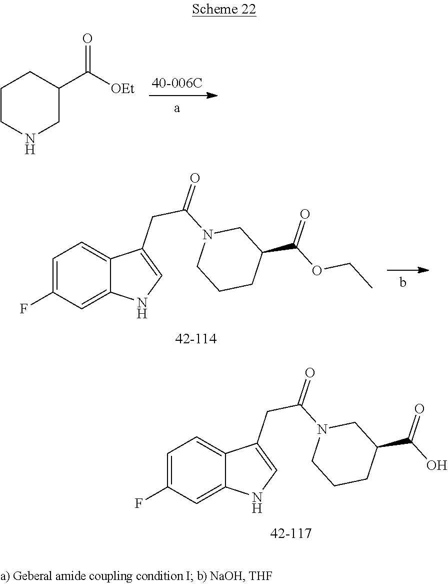 [SMILES: CCOC(=O)C1CCCNC1 ; CCOC(=O)[C@H]1CCCN(C(=O)Cc2c[nH]c3cc(F)ccc23)C1 ; O=C(O)[C@H]1CCCN(C(=O)Cc2c[nH]c3cc(F)ccc23)C1]

### ethyl (S)-1-(2-(6-fluoro-1H-indol-3-yl) acetyl) piperidine-3-carboxylate (42-114)

Following general amide coupling condition I, the desired product was obtained as yellow solid (149 mg, 43% yield).

MS (ESI+) m/z 333 (M+H)⁺.

¹H NMR (400 MHz, DMSO-d₆) δ 10.96 (s, 1H), 7.55-7.47 (m, 1H), 7.23-7.16 (m, 1H), 7.15-7.06 (m, 1H), 6.88-6.78 (m, 1H), 4.38-4.30 (m, 0.5H), 4.10-4.00 (m, 2H), 3.95-3.80 (m, 2H), 3.77-3.64 (m, 1.5H), 3.32-3.16 (m, 0.5H), 3.08-2.78 (m, 1.5H), 2.33-2.14 (m, 1H), 1.91-1.78 (m, 1H), 1.65-1.48 (m, 2H), 1.32-1.13 (m, 4H).

### (S)-1-(2-(6-fluoro-1H-indol-3-yl)acetyl)piperidine-3-carboxylic acid (42-117) Compound #10133

To the solution of ethyl (S)-1-(2-(6-fluoro-1H-indol-3-yl)acetyl)piperidine-3-carboxylate (79 mg, 0.24 mmol) in THE (1 mL) was added sodium hydroxide (1 mL, 1 N) and the mixture was stirred at room temperature for 4 hours. Then, the solvent was evaporated under reduced pressure and the residue was adjusted pH below 7 with HCl (1N, 3 mL). The aqueous mixture was extracted with EtOAc (20 mL*3). The combined organic layers were dried over sodium sulfate, filtered and concentrated dryness. The residue was purified by pre. HPLC to obtain desired product as white solid (41.3 mg, 57% yield).

MS (ESI+) m/z 305 (M+H)⁺.

¹H NMR (400 MHz, DMSO-d₆) δ 10.96 (s, 1H), 7.57-7.48 (m, 1H), 7.21-7.08 (m, 2H), 6.87-6.78 (m, 1H), 4.46-4.37 (m, 0.5H), 3.95-3.68 (m, 3.5H), 3.34-3.24 (m, 0.5H), 3.02-2.85 (m, 1H), 2.76-2.66 (m, 0.5H), 2.22-2.17 (m, 1H), 1.93-1.79 (m, 1H), 1.66-1.42 (m, 2H), 1.33-1.05 (m, 1H).

### Example 31—Synthesis of Further Compounds Using the Procedure of Example 30

Using the above procedures, the following compounds were synthesized:

TABLE 9

| | Compound | | LC-MS |
|---|---|---|---|
| Structure | # | ¹H NMR | (M + H)⁺ |
| 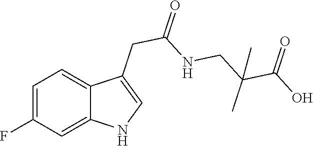 [SMILES: CC(C)(CNC(=O)Cc1c[nH]c2cc(F)ccc12)C(=O)O] | 10062 | ¹H NMR (400 MHz, DMSO-d₆) δ 12.25 (br s, 1H), 10.92 (s, 1H), 7.70 (t, J = 6.1 Hz, 1H), 7.55- 7.49 (m, 1H), 7.20-7.15 (m, 1H), 7.12-7.07 (m, 1H), 6.86-6.79 (m, 1H), 3.53 (s, 2H), 3.20 (d, J = 6.2 Hz, 2H), 1.01 (s, 6H). | 293 |
| 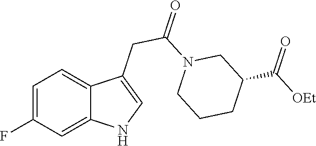 [SMILES: CCOC(=O)[C@@H]1CCCN(C(=O)Cc2c[nH]c3cc(F)ccc23)C1] | 10161 | ¹H NMR (400 MHz, DMSO-d₆) δ 10.97 (s, 1H), 7.57-7.49 (m, 1H), 7.21-7.07 (m, 2H), 6.87- 6.78 (m, 1H), 4.38-4.31 (m, 0.5H), 4.10-4.00 (m, 2H), 3.95-3.80 (m, 2H), 3.78-3.67 (m, 1.5H), 3.31-3.27 (m, 0.5H), 3.07-2.77 (m, 1.5H), 2.34-2.15 (m, 1H), 1.91-1.78 (m, 1H), 1.66-1.48 (m, 2H), 1.33-1.13 (m, 4H). | 333 |
| 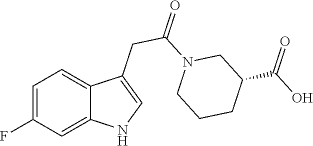 [SMILES: O=C(O)[C@@H]1CCCN(C(=O)Cc2c[nH]c3cc(F)ccc23)C1] | 10149 | ¹H NMR (400 MHz, DMSO-d₆) δ 10.96 (s, 1H), 7.56-7.47 (m, 1H), 7.23-7.07 (m, 2H), 6.88- 6.78 (m, 1H), 4.45-4.37 (m, 0.5H), 3.99-3.69 (m, 3.5H), 3.36-3.24 (m, 0.5H), 3.04-2.84 (m, 1H), 2.75-2.66 (m, 0.5H), 2.25-2.11 (m, 1H), 1.95-1.79 (m, 1H), 1.69-1.42 (m, 2H), 1.35- 1.07 (m, 1H). | 305 |
| 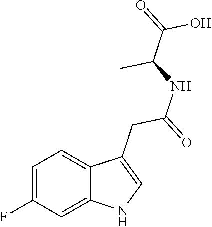 [SMILES: C[C@H](NC(=O)Cc1c[nH]c2cc(F)ccc12)C(=O)O] | 10084 | ¹H NMR (400 MHz, DMSO-d₆) δ 10.99 (s, 1H), 10.19 (s, 1H), 7.80 (d, J = 2.0 Hz, 1H), 7.61- 7.53 (m, 1H), 7.40-7.34 (m, 1H), 7.29-7.22 (m, 2H), 7.15-7.08 (m, 1H), 6.90-6.81 (m, 1H), 3.70 (s, 2H), 2.25 (s, 3H). | 265 |
| 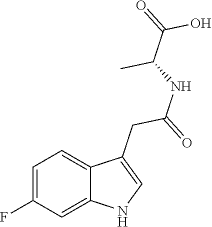 [SMILES: C[C@@H](NC(=O)Cc1c[nH]c2cc(F)ccc12)C(=O)O] | 10083 | ¹H NMR (400 MHz, DMSO-d₆) δ 10.99 (s, 1H), 10.19 (s, 1H), 7.80 (d, J = 2.0 Hz, 1H), 7.61- 7.53 (m, 1H), 7.40-7.34 (m, 1H), 7.29-7.22 (m, 2H), 7.15-7.08 (m, 1H), 6.90-6.81 (m, 1H), 3.70 (s, 2H), 2.25 (s, 3H). | 265 |
| 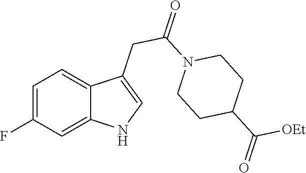 [SMILES: CCOC(=O)C1CCN(C(=O)Cc2c[nH]c3cc(F)ccc23)CC1] | 10132 | 1H NMR (400 MHz, CDCl₃) δ 8.16 (s, 1H), 7.58- 7.50 (m, 1H), 7.07-6.99 (m, 2H), 6.94-6.84 (m, 1H), 4.51-4.39 (m, 1H), 4.12 (q, J = 7.1 Hz, 2H), 3.94-3.76 (m, 3H), 3.15-3.03 (m, 1H), 2.92-2.78 (m, 1H), 2.53-2.40 (m, 1H), 1.97- 1.86 (m, 1H), 1.84-1.71 (m, 1H), 1.68-1.54 (m, 1H), 1.53-1.38 (m, 1H), 1.23 (t, J = 7.1 Hz, 3H). | 333 |
| 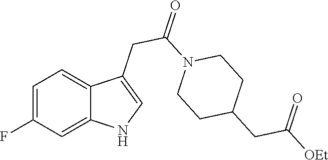 [SMILES: CCOC(=O)CC1CCN(C(=O)Cc2c[nH]c3cc(F)ccc23)CC1] | 10130 | ¹H NMR (400 MHz, DMSO-d₆) δ 10.94 (s, 1H), 7.58-7.47 (m, 1H), 7.23-7.15 (m, 1H), 7.14- 7.04 (m, 1H), 6.87-6.77 (m, 1H), 4.41-4.30 (m, 1H), 4.08-3.93 (m, 3H), 3.73 (s, 2H), 3.01- 2.89 (m, 1H), 2.56-2.51 (m, 1H), 2.19-2.12 (m, 2H), 1.93-1.78 (m, 1H), 1.66-1.52 (m, 2H), 1.16 (t, J = 7.1 Hz, 3H), 1.03-0.80 (m, 2H). | 347 |
| 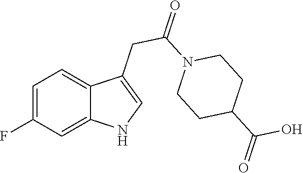 [SMILES: O=C(O)C1CCN(C(=O)Cc2c[nH]c3cc(F)ccc23)CC1] | 10129 | ¹H NMR (400 MHz, DMSO-d₆) δ 12.21 (s, 1H), 10.95 (s, 1H), 7.58-7.48 (m, 1H), 7.22-7.15 (m, 1H), 7.14-7.04 (m, 1H), 6.88-6.76 (m, 1H), 4.29-4.16 (m, 1H), 4.00-3.85 (m, 1H), 3.77-3.71 (m, 2H), 3.11-3.00 (m, 1H), 2.76- 2.64 (m, 1H), 2.48-2.36 (m, 1H), 1.82-1.63 (m, 2H), 1.37-1.15 (m, 2H). | 305 |
| 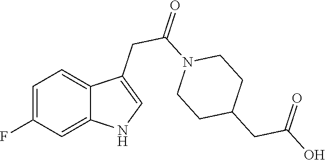 [SMILES: O=C(O)CC1CCN(C(=O)Cc2c[nH]c3cc(F)ccc23)CC1] | 10127 | ¹H NMR (400 MHz, DMSO-d₆) δ 12.12 (br s, 1H), 10.94 (s, 1H), 7.57-7.48 (m, 1H), 7.22-7.16 (m, 1H), 7.14-7.05 (m, 1H), 6.88-6.77 (m, 1H), 4.42-4.31 (m, 1H), 4.05-3.89 (m, 1H), 3.73 (s, 2H), 3.01-2.88 (m, 1H), 2.58-2.52 (m, 1H), 2.10-2.03 (m, 2H), 1.91-1.75 (m, 1H), 1.69-1.52 (m, 2H), 1.01-0.77 (m, 2H). | 319 |
| 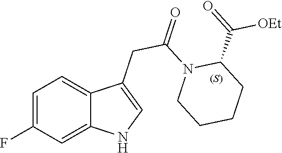 | 10126 | ¹H NMR (400 MHz, DMSO-d₆) δ 11.01-10.89 (m, 1H), 7.55-7.46 (m, 1H), 7.24-7.15 (m, 1H), 7.14-7.05 (m, 1H), 6.89-6.77 (m, 1H), 5.21-5.12 (m, 0.7H), 5.01-4.95 (m, 0.3H), 4.42-4.32 (m, 0.3H), 4.14-3.68 (m, 5H), 3.12- 2.99 (m, 0.7H), 2.16-2.00 (m, 1H), 1.66- 1.44 (m, 3H), 1.28-1.03 (m, 5H). | 333 |
| 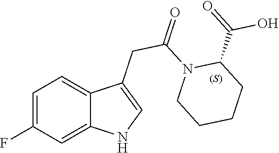 | 10123 | ¹H NMR (400 MHz, DMSO-d₆) δ 12.79 (s, 1H), 11.00-10.86 (m, 1H), 7.54-7.44 (m, 1H), 7.25- 7.17 (m, 1H), 7.15-7.06 (m, 1H), 6.89-6.77 (m, 1H), 5.18-5.07 (m, 0.7H), 4.90-4.85 (m, 0.3H), 4.41-4.31 (m, 0.3H), 3.96-3.55 (m, 2.7H), 3.21-3.06 (m, 0.7H), 2.60-2.53 (m, 0.3H), 2.17-1.95 (m, 1H), 1.64-1.11 (m, 5H). | 305 |

### Example 32—Synthesis of 2-(4-(2-(6-fluoro-1H-indol-3-yl)acetamido)piperidin-1-yl)acetic acid (Compound #10108) (41-100 in Scheme 23)

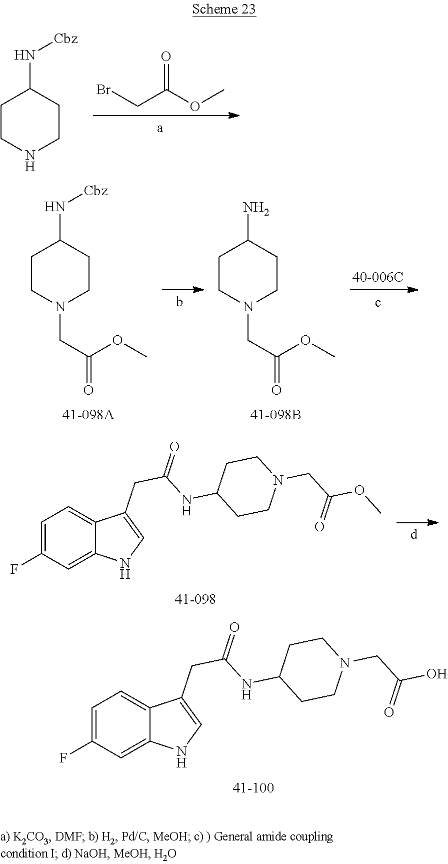

### Methyl 2-(4-(((benzyloxy)carbonyl) amino) piperidin-1-yl) acetate (41-098A)

The mixture of benzyl piperidin-4-ylcarbamate (500 mg, 2.13 mmol), methyl 2-bromoacetate (360 mg, 2.35 mmol) and K₂CO₃ (589 mg, 0.42 mmol) in DMF (20 mL) was stirred at room temperature for 12 hours. After which period, the reaction was diluted with water (40 mL), extracted with EtOAc (15 mL*3). The combined organic layers were washed with brine (30 mL), dried over sodium sulfate, filtered and concentrated to dryness. The residue was purified by column flash to get methyl 2-(4-(((benzyloxy)carbonyl) amino) piperidin-1-yl) acetate (695 mg, 96.6%) as colorless oil.

MS (ESI+) m/z 307 (M+H)⁺.

### Methyl 2-(4-aminopiperidin-1-yl) acetate (41-098B)

To a solution of methyl 2-(4-(((benzyloxy)carbonyl) amino) piperidin-1-yl) acetate (695 mg, 2.26 mmol) in MeOH (15 mL) was added Pd/C (218 mg, 2.04 mmol), and the mixture was stirred under H₂ at room temperature for 12 hours. The mixture was filtered through celite and the filtrate was concentrated under reduced pressure to get methyl 2-(4-aminopiperidin-1-yl)acetate (363 mg, 92.9% yield) as colorless oil.

MS (ESI+) m/z 173 (M+H)⁺.

### Methyl 2-(4-(2-(6-fluoro-1H-indol-3-yl)acetamido)piperidin-1-yl)acetate (41-098)

Following general amide coupling condition I, the desired product was obtained as white solid (120 mg, 33.4% yield).

MS (ESI+) m/z 348 (M+H)⁺.

¹H NMR (400 MHz, DMSO-d₆) δ 10.90 (s, 1H), 7.95-7.87 (m, 1H), 7.57-7.48 (m, 1H), 7.18-7.13 (m, 1H), 7.13-7.06 (m, 1H), 6.88-6.78 (m, 1H), 3.60 (s, 3H), 3.52-3.43 (m, 3H), 3.19 (s, 2H), 2.80-2.71 (m, 2H), 2.24-2.14 (m, 2H), 1.67 (d, J=9.6 Hz, 2H), 1.47-1.31 (m, 2H).

### 2-(4-(2-(6-fluoro-1H-indol-3-yl)acetamido)piperidin-1-yl)acetic acid (41-100) Compound #10108

Methyl 2-(4-(2-(6-fluoro-1H-indol-3-yl)acetamido)piperidin-1-yl)acetate (90 mg, 0.31 mmol) was dissolved in MeOH/H₂O=10:1 (5.5 mL), after NaOH (1 N, 1 mL) added dropwise, the mixture was stirred at room temperature for 12 hours. HCl (1.0 N) was then added to above solution dropwise till the pH=3. The precipitate was filtered and washed with water (5 mL) to get 2-(4-(2-(6-fluoro-1H-indol-3-yl)acetamido)piperidin-1-yl)acetic acid (74.2 mg, 77.3% yield) as white solid.

MS (ESI+) m/z 334 (M+H)⁺.

¹H NMR (400 MHz, DMSO-d₆) δ 10.95 (s, 1H), 8.16 (d, J=7.3 Hz, 1H), 7.58-7.47 (m, 1H), 7.20-7.14 (m, 1H), 7.14-7.06 (m, 1H), 6.89-6.78 (m, 1H), 3.96 (s, 2H), 3.81-3.70 (m, 1H), 3.52-3.46 (m, 2H), 3.46-3.37 (m, 2H), 3.14-3.02 (m, 2H), 1.98-1.88 (m, 2H), 1.79-1.63 (m, 2H).

### Example 33—Synthesis of (S)-4-(2-(6-fluoro-1H-indol-3-yl)acetyl)morpholine-3-carboxylic acid (Compound #10150) (40-105 in Scheme 24)

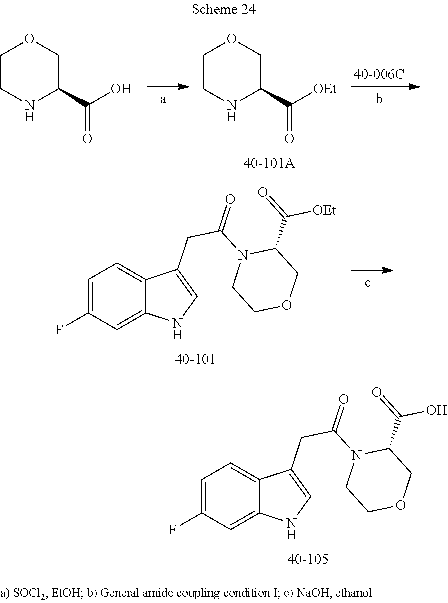 [SMILES: O=C(O)[C@@H]1COCCN1 ; CCOC(=O)[C@@H]1COCCN1 ; CCOC(=O)[C@@H]1COCCN1C(=O)Cc1c[nH]c2cc(F)ccc12 ; O=C(O)[C@@H]1COCCN1C(=O)Cc1c[nH]c2cc(F)ccc12]

### (S)-2-((morpholine-3-carbonyl) oxy) ethan-1-ylium (40-101A)

The solution of (S)-morpholine-3-carboxylic acid (150 mg, 1.14 mmol) in ethanol (5 mL) was added thionyl chloride (0.25 mL, 3.43 mmol) dropwise at 0° C. Then, the mixture was refluxed for 6 hours. After cooling to room temperature, the mixture was concentrated to get crude product for the next step without further purification.

MS (ESI+) m/z 160 (M+H)⁺.

### ethyl (S)-4-(2-(6-fluoro-1H-indol-3-yl) acetyl) morpholine-3-carboxylate (40-101) (Compound #10140)

Following general amide coupling condition I, the desired product was obtained as white solid (300 mg, 86.7% yield).

MS (ESI+) m/z 335 (M+H)⁺.

¹H NMR (400 MHz, CDCl₃) δ 8.11 (s, 1H), 7.57-7.44 (m, 1H), 7.19 (s, 1H), 7.08-7.00 (m, 1H), 6.97-6.83 (m, 1H), 5.16-5.11 (m, 1H), 4.48-4.41 (m, 1H), 4.38-3.07 (m, 9H), 1.26 (t, J=7.1 Hz, 2.3H), 1.15 (t, J=7.1 Hz, 0.7H).

### (S)-4-(2-(6-fluoro-1H-indol-3-yl)acetyl)morpholine-3-carboxylic acid (40-105) (Compound #10150)

A solution of ethyl (S)-4-(2-(6-fluoro-1H-indol-3-yl) acetyl)morpholine-3-carboxylate (200 mg, 0.60 mmol) in ethanol (10 mL) was added 1 N sodium hydroxide (10 mL) and stirred at room temperature for 16 hours. The solvent was removed under reduced pressure and the residual aqueous solution was neutralized with conc. Hydrochloride acid. The precipitate was filtered and washed with water (2 mL*2). The solid was further purified by Prep-HPLC to obtain (S)-4-(2-(6-fluoro-1H-indol-3-yl)acetyl)morpholine-3-carboxylic acid as white solid (82 mg, 44.8% yield).

MS (ESI+) m/z 307 (M+H)⁺.

¹H NMR (400 MHz, DMSO-d₆) δ 13.11 (s, 1H), 10.97-10.92 (m, 1H), 7.50-7.45 (m, 1H), 7.24-7.20 (m, 1H), 7.12-7.08 (m, 1H), 6.92-6.75 (m, 1H), 4.87-4.73 (m, 1H), 4.41-3.16 (m, 7.7H), 2.87-2.80 (m, 0.3H).

### Example 34—Synthesis of Further Compounds Using the Procedure of Example 33

Using the above procedures, the following compounds were synthesized:

TABLE 10

| | Compound | | LC-MS |
|---|---|---|---|
| Structure | # | ¹H NMR | (M + H)⁺ |
| 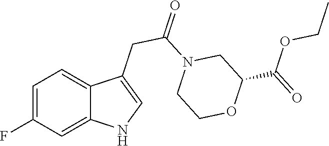 [SMILES: CCOC(=O)[C@H]1CN(C(=O)Cc2c[nH]c3cc(F)ccc23)CCO1] | 10169 | ¹H NMR (400 MHz, CDCl₃) δ 8.24 (s, 1H), 7.58- 7.51 (m, 1H), 7.13-6.98 (m, 2H), 6.92-6.87 (m, 1H), 4.62-4.59 (m, 0.5H), 4.34-3.62 (m, 7.5H), 3.59-2.87 (m, 3H), 1.30-1.25 (m, 3H). | 335 |
| 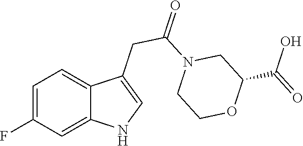 [SMILES: O=C(O)[C@H]1CN(C(=O)Cc2c[nH]c3cc(F)ccc23)CCO1] | 10153 | ¹H NMR (400 MHz, DMSO-d₆) δ 12.97 (s, 1H), 10.97 (s, 1H), 7.54-7.48 (m, 1H), 7.34-6.98 (m, 2H), 6.97-6.73 (m, 1H), 4.40-3.64 (m, 6H), 3.58-2.92 (m, 3H). | 307 |
| 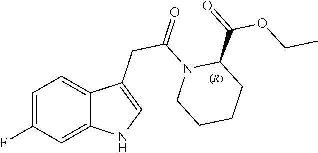 | 10128 | ¹H NMR (400 MHz, DMSO-d₆) δ 11.02-10.89 (m, 1H), 7.58-7.44 (m, 1H), 7.24-7.16 (m, 1H), 7.15-7.06 (m, 1H), 6.88-6.77 (m, 1H), 5.21-5.12 (m, 0.7H), 5.02-4.93 (m, 0.3H), 4.43-4.31 (m, 0.3H), 4.19-3.68 (m, 5H), 3.13- 3.00 (m, 0.7H), 2.16-2.00 (m, 1H), 1.67- 1.41 (m, 3H), 1.29-1.03 (m, 5H). | 333 |
| 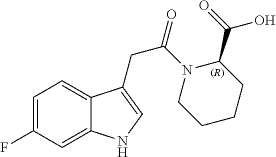 | 10125 | ¹H NMR (400 MHz, DMSO-d₆) δ 12.90-12.70 (m, 1H), 11.05-10.84 (m, 1H), 7.53-7.44 (m, 1H), 7.24-7.18 (m, 1H), 7.13-7.06 (m, 1H), 6.87-6.77 (m, 1H), 5.18-5.08 (m, 0.7H), 4.93- 4.84 (m, 0.3H), 4.40-4.26 (m, 0.3H), 3.97- 3.50 (m, 2.7H), 3.21-3.03 (m, 0.7H), 2.62- 2.54 (m, 0.3H), 2.16-2.00 (m, 1H), 1.67-1.09 (m, 5H). | 305 |
| 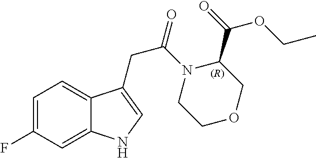 | 10121 | ¹H NMR (400 MHz, CDCl₃) δ 8.20 (s, 1H), 7.54- 7.43 (m, 1H), 7.19-7.13 (m, 1H), 7.07-6.98 (m, 1H), 6.94-6.85 (m, 1H), 5.17-5.09 (m, 1H), 4.49-4.39 (m, 1H), 4.39-3.73 (m, 5H), 3.66-3.32 (m, 3.7H), 3.19-3.06 (m, 0.3H), 1.26 (t, J = 7.1 Hz, 2.3H), 1.15 (t, J = 7.1 Hz, 0.7H). | 335 |
| 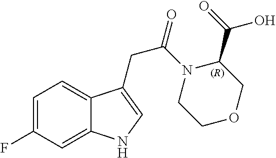 | 10152 | ¹H NMR (400 MHz, DMSO-d₆) δ 12.98 (br s, 1H), 11.04-10.85 (m, 1H), 7.54-7.42 (m, 1H), 7.28- 7.18 (m, 1H), 7.14-7.05 (m, 1H), 6.89-6.75 (m, 1H), 4.87-4.75 (m, 1H), 4.28-4.15 (m, 1H), 4.10-4.00 (m, 0.3H), 3.89-3.24 (m, 6.4H), 2.91-2.77 (m, 0.3H). | 307 |
| 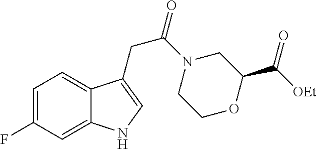 [SMILES: CCOC(=O)[C@@H]1CN(C(=O)Cc2c[nH]c3cc(F)ccc23)CCO1] | 10168 | ¹H NMR (400 MHz, CDCl₃) δ 8.23 (s, 1H), 7.60- 7.49 (m, 1H), 7.10-7.00 (m, 2H), 6.94-6.84 (m, 1H), 4.64-4.57 (m, 0.5H), 4.28-4.16 (m, 2H), 4.14-3.81 (m, 5H), 3.72-3.64 (m, 0.5H), 3.58-3.42 (m, 1H), 3.39-3.15 (m, 1.5H), 3.12- 3.04 (m, 0.5H), 1.34-1.20 (m, 3H). | 335 |
| 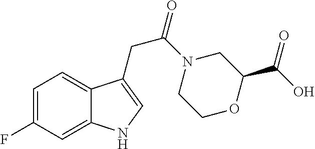 [SMILES: O=C(O)[C@@H]1CN(C(=O)Cc2c[nH]c3cc(F)ccc23)CCO1] | 10151 | ¹H NMR (400 MHz, DMSO-d₆) δ 13.04 (br s, 1H), 10.97 (s, 1H), 7.55-7.44 (m, 1H), 7.23-7.15 (m, 1H), 7.14-7.06 (m, 1H), 6.88-6.76 (m, 1H), 4.24-4.17 (m, 1H), 4.02-3.63 (m, 5H), 3.49-3.33 (m, 1.5H), 3.29-3.11 (m, 1H), 3.05- 2.99 (m, 0.5H). | 307 |

### Example 35—Synthesis of (R)-2-(6-fluoro-1H-indol-3-yl)-1-(2-(methoxymethyl)morpholino)ethan-1-one (Compound #10154) (42-127 in Scheme 25)

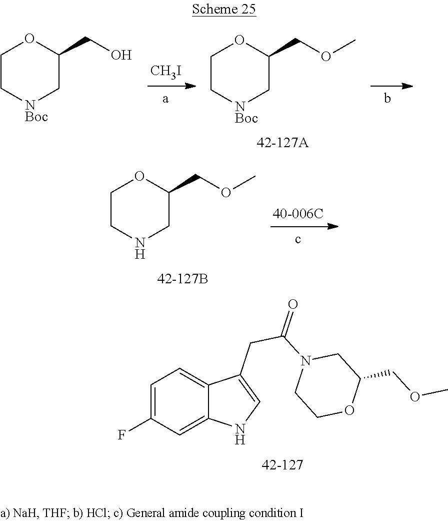

### tert-butyl-(R)-2-(methoxymethyl) morpholine-4-carboxylate (42-127A)

To the solution of tert-butyl (R)-2-(hydroxymethyl) morpholine-4-carboxylate (600 mg, 2.76 mmol) in THE (18 mL) was added NaH (221.0 mg, 5.52 mmol) by portions at 0° C. After stirring at 0° C. for 20 minutes, iodomethane (588 mg, 4.14 mmol) was added thereto and the mixture was stirred at room temperature for 16 hours. Then, the reaction was quenched with water (50 mL), extracted with EtOAc (60 mL), washed with brine (30 mL), dried over sodium sulfate. After filtration and concentration to dryness, the residue was purified by flash column to obtain desired product as yellow oil (490 mg, 77% yield)

MS (ESI+) m/z 132 (M+H)⁺.

### (R)-2-(methoxymethyl) morpholine (42-127B)

The solution of tert-butyl (R)-2-(methoxymethyl) morpholine-4-carboxylate (490 mg, 2.12 mmol) in HCl (8 mL, 4.0 M in methanol) was stirred at room temperature for 4 hours. Then, the mixture was concentrated to get crude product, which was used in the next step without further purification.

MS (ESI+) m/z 132 (M+H)⁺.

### (R)-2-(6-fluoro-1H-indol-3-yl)-1-(2-(methoxymethyl)morpholino)ethan-1-one (42-127)(Compound #10154)

Following general amide coupling condition I, the desired product was obtained as white solid (54.8 mg, 34.6% yield).

MS (ESI+) m/z 307 (M+H)⁺.

¹H NMR (400 MHz, CDCl₃) δ 8.22 (s, 1H), 7.58-7.49 (m, 1H), 7.08-6.98 (m, 2H), 6.95-6.80 (m, 1H), 4.54-4.39 (m, 1H), 3.94-3.68 (m, 4H), 3.55-3.17 (m, 7.5H), 3.06-2.99 (m, 0.5H), 2.84 (s, 0.5H), 2.69-2.60 (m, 0.5H).

### Example 36—Synthesis of Further Compounds Using the Procedure of Example 35

Using the above procedures, the following compounds were synthesized:

TABLE 11

| | Compound | | LC-MS |
|---|---|---|---|
| Structure | # | ¹H NMR | (M + H)⁺ |
| 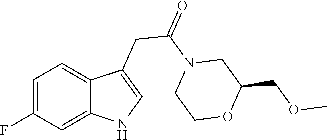 [SMILES: COC[C@@H]1CN(C(=O)Cc2c[nH]c3cc(F)ccc23)CCO1] | 10155 | ¹H NMR (400 MHz, CDCl₃) δ 8.29 (s, 1H), 7.60 − 7.47 (m, 1H), 7.08 − 6.98 (m, 2H), 6.94 − 6.84 (m, 1H), 4.56 − 4.36 (m, 1H), 4.00 − 3.66 (m, 4H), 3.61 − 3.13 (m, 7.5H), 3.10 − 2.97 (m, 0.5H), 2.92 − 2.79 (m, 0.5H), 2.72 − 2.58 (m, 0.5H). | 307 |

### Example 37—Synthesis of Compounds #10147 and 10160 (42-129 and 42-138 in Scheme 26, respectively)

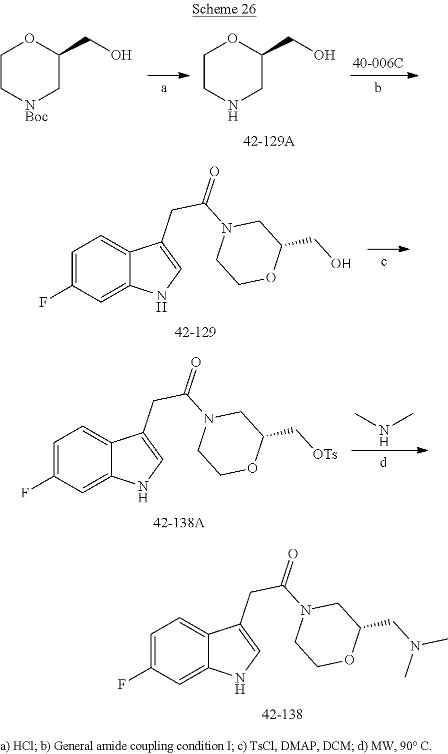

### (R)-morpholin-2-ylmethanol (42-129A)

The solution of tert-butyl (R)-2-(hydroxymethyl) morpholine-4-carboxylate (3 g, 2.12 mmol) in HCl (20 mL, 4.0 M in methanol) was stirred at room temperature for 4 hours. Then, the mixture was concentrated to get crude product, which was used in the next step without further purification.

MS (ESI+) m/z 118 (M+H)⁺.

### (R)-2-(6-fluoro-1H-indol-3-yl)-1-(2-(hydroxymethyl) morpholino) ethan-1-one (42-129) (Compound #10147)

Following general amide coupling condition I, the desired product was obtained as white solid (2.18 g, 96% yield).

MS (ESI+) m/z 293 (M+H)⁺.

¹H NMR (400 MHz, CDCl₃) δ 8.31 (s, 1H), 7.58-7.48 (m, 1H), 7.07-6.98 (m, 2H), 6.94-6.84 (m, 1H), 4.52-4.42 (m, 1H), 4.04-3.39 (m, 8H), 3.35-3.16 (m, 1.5H), 3.09-3.01 (m, 0.5H), 2.86-2.77 (m, 0.5H), 2.70-2.62 (m, 0.5H).

### (R)-(4-(2-(6-fluoro-1H-indol-3-yl)acetyl)morpholin-2-yl)methyl 4-methylbenzenesulfonate (42-138A)

The solution of (R)-2-(6-fluoro-1H-indol-3-yl)-1-(2-(hydroxymethyl)morpholino)ethan-1-one (1.42 g, 4.87 mmol), Et₃N (0.95 g, 9.75 mmol) and DMAP (0.12 g, 0.97 mmol) in DCM (20 mL) was cooled to 0° C. TsCI (1.02 g, 5.36 mmol) was added to above solution. And then, the reaction was stirred at room temperature for 16 hours. After which period, the mixture was diluted with DCM (50 mL), washed with water (50 mL), dried over sodium sulfate. After filtration and concentration to dryness, the residue was purified by flash column to obtain desired product as yellow oil (1.05 g, 48% yield).

MS (ESI+) m/z 447 (M+H)⁺.

### (S)-1-(2-((dimethylamino)methyl) morpholino)-2-(6-fluoro-1H-indol-3-yl) ethan-1-one (42-138) (Compound #10160)

The solution of (R)-(4-(2-(6-fluoro-1H-indol-3-yl) acetyl)morpholin-2-yl)methyl 4-methylbenzenesulfonate (200 mg, 0.45 mmol) and dimethylamine (4 mL, 2.0 M in EtOH) was reacted in microwave reactor at 90° C. for 1 hour. After cooling to room temperature, the mixture was concentrated to dryness, and the resulting residue was purified by Prep-HPLC to obtain title compound as white solid (96 mg, 67% yield)

MS (ESI+) m/z 320 (M+H)⁺. 320

¹H NMR (400 MHz, DMSO-d₆) δ 11.02-10.92 (m, 1H), 7.58-7.49 (m, 1H), 7.22-7.17 (m, 1H), 7.15-7.07 (m, 1H), 6.90-6.79 (m, 1H), 4.34-4.26 (m, 0.5H), 4.22-4.16 (m, 0.5H), 3.94-3.65 (m, 4H), 3.30-3.02 (m, 2H), 2.88-2.77 (m, 0.5H), 2.74-2.64 (m, 0.5H), 2.45-2.06 (m, 6H), 1.99 (s, 3H).

### Example 38—Synthesis of Further Compounds Using the Procedure of Example 37

Using the above procedures, the following compounds were synthesized:

TABLE 12

| | Compound | | LC-MS |
|---|---|---|---|
| Structure | # | ¹H NMR | (M + H)⁺ |
| 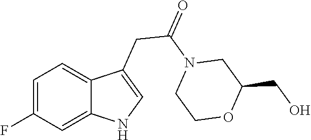 [SMILES: O=C(Cc1c[nH]c2cc(F)ccc12)N1CCO[C@H](CO)C1] | 10148 | ¹H NMR (400 MHz, CDCl₃) δ 8.19 (s, 1H), 7.58 − 7.47 (m, 1H), 7.07 − 6.99 (m, 2H), 6.95 − 6.84 (m, 1H), 4.54 − 4.40 (m, 1H), 3.98 − 3.65 (m, 4.5H), 3.63 − 3.40 (m, 2.5H), 3.39 − 3.26 (m, 0.5H), 3.25 − 3.13 (m, 1H), 3.11 − 2.98 (m, 0.5H), 2.88 − 2.76 (m, 0.5H), 2.73 − 2.61 (m, 0.5H). | 293 |
| 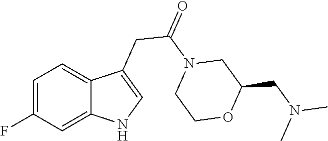 [SMILES: CN(C)C[C@@H]1CN(C(=O)Cc2c[nH]c3cc(F)ccc23)CCO1] | 10159 | ¹H NMR (400 MHz, CDCl₃) δ 8.26 (s, 1H), 7.61 − 7.48 (m, 1H), 7.11 − 6.98 (m, 2H), 6.96 − 6.83 (m, 1H), 4.54 − 4.37 (m, 1H), 3.94 − 3.76 (m, 3.5H), 3.75 − 3.67 (m, 0.5H), 3.54 − 3.38 (m, 1H), 3.34 − 3.25 (m, 0.5H), 3.24 − 3.13 (m, 1H), 2.91 − 2.76 (m, 1H), 2.53 − 2.43 (m, 1H), 2.37 − 2.17 (m, 4H), 2.16 − 2.03 (m, 3.5H). | 320 |

### Example 39—Synthesis of (S)—N-((4-(2-(6-fluoro-1H-indol-3-yl) acetyl) morpholin-2-yl) methyl) acetamide (Compound #10162) (42-146 in Scheme 27)

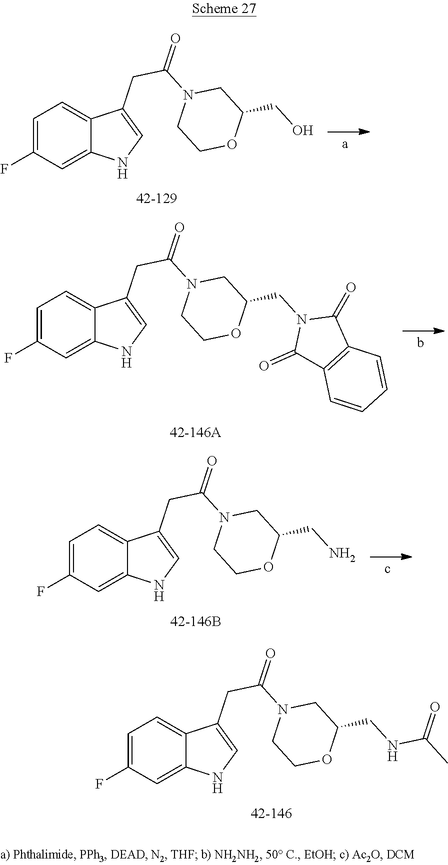 [SMILES: O=C(Cc1c[nH]c2cc(F)ccc12)N1CCO[C@@H](CO)C1 ; O=C(Cc1c[nH]c2cc(F)ccc12)N1CCO[C@@H](CN2C(=O)c3ccccc3C2=O)C1 ; NC[C@H]1CN(C(=O)Cc2c[nH]c3cc(F)ccc23)CCO1 ; CC(=O)NC[C@H]1CN(C(=O)Cc2c[nH]c3cc(F)ccc23)CCO1]

### (R)-2-((4 (2-(6-fluoro-1H-indol-3-yl)acetyl)morpholin-2-yl)methyl)isoindoline-1,3-dione (42-146A)

DEAD (0.9 mL, 2.30 mmol) was added dropwise to the solution of (R)-2-(6-fluoro-1H-indol-3-yl)-1-(2-(hydroxymethyl)morpholino)ethan-1-one (516 mg, 1.77 mmol), phthalimide (311.7 mg, 2.12 mmol) and PPh₃ (1.39 g, 2.30 mmol) in THE (15 mL) at 0° C. After addition, the mixture was slowly warmed to room temperature and continued to stir for 1 hour. After then, the mixture was concentrated to dryness, which was purified by flash column to obtain the titled compound as yellow oil (689 mg, 93% yield).

MS (ESI+) m/z 422 (M+H)⁺.

### (S)-1-(2-(aminomethyl)morpholino)-2-(6-fluoro-1H-indol-3-yl)ethan-1-one (42-146B)

The solution of (R)-2-((4-(2-(6-fluoro-1H-indol-3-yl)acetyl)morpholin-2-yl)methyl)isoindoline-1,3-dione (689 mg, 1.63 mmol) and N₂H₄ (115.4 mg, 3.60 mmol) in dry EtOH (15 mL) was stirred at 50° C. for 3 hours. After which period, the reaction was concentrated diluted with brine (50 mL). The resulting mixture was extracted with EA (50 mL*2), and the combined organic layers were dried over sodium sulfate. After filtration and concentration to dryness, the residue was purified by Prep-HPLC to obtain desired product as white solid (88 mg, 18% yield).

MS (ESI+) m/z 292 (M+H)⁺.

### (S)—N-((4-(2-(6-fluoro-1H-indol-3-yl) acetyl) morpholin-2-yl) methyl) acetamide (42-146) Compound #10162

The solution of (S)-1-(2-(aminomethyl)morpholino)-2-(6-fluoro-1H-indol-3-yl)ethan-1-one (88 mg, 0.30 mmol), acidic anhydride (0.05 mL, 0.51 mmol) and Et₃N (65 mg, 0.64 mmol) in DCM (4 mL) was stirred at room temperature for 2 hours. Then, the mixture was treated with saturated aqueous NaHCO₃ (20 mL), and then extracted with DCM (40 mL). The combined organic layers were washed with brine (20 mL), dried over sodium sulfate. After filtration and concentration to dryness, the residue was purified by Prep-HPLC to get desired product as white solid (41 mg, 39% yield).

MS (ESI+) m/z 334 (M+H)⁺.

¹H NMR (400 MHz, CDCl₃) δ 8.71 (s, 1H), 7.59-7.42 (m, 1H), 7.04-6.93 (m, 2H), 6.91-6.84 (m, 1H), 5.98-5.79 (m, 1H), 4.52-4.40 (m, 1H), 3.88-3.67 (m, 4H), 3.50-3.35 (m, 2H), 3.31-3.00 (m, 2.5H), 2.92-2.84 (m, 0.5H), 2.81-2.72 (m, 0.5H), 2.56-2.48 (m, 0.5H), 2.01-1.93 (m, 3H).

### Example 40—Synthesis of Further Compounds Using the Procedure of Example 39

Using the above procedures, the following compounds were synthesized:

TABLE 13

| | Com- | | |
|---|---|---|---|
| | pound | | LC-MS |
| Structure | # | ¹H NMR | (M + H)⁺ |
| 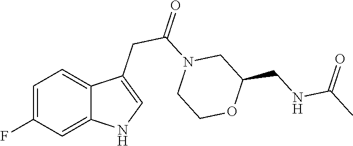 [SMILES: CC(=O)NC[C@@H]1CN(C(=O)Cc2c[nH]c3cc(F)ccc23)CCO1] | 10164 | ¹H NMR (400 MHz, CDCl₃) δ 8.45-8.28 (m, 1H), 7.62-7.47 (m, 1H), 7.07-6.99 (m, 2H), 6.94-6.83 (m, 1H), 5.83-5.70 (m, 1H), 4.56- 4.39 (m, 1H), 3.92-3.66 (m, 4H), 3.56- 3.34 (m, 2H), 3.33-2.99 (m, 2.5H), 2.93- 2.84 (m, 0.5H), 2.82-2.72 (m, 0.5H), 2.60- 2.48 (m, 0.5H), 2.02-1.93 (m, 3H). | 334 |

### Example 41—Synthesis of Compounds #10177 and 10178 (70-022 and 70-027 in Scheme 28)

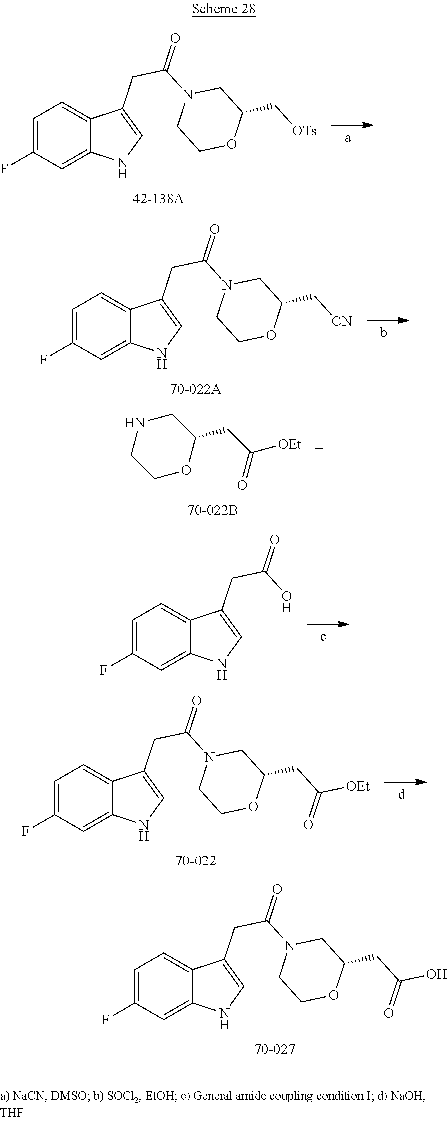

### (S)-2-(4-(2-(6-fluoro-1H-indol-3-yl) acetyl) morpholin-2-yl) acetonitrile (70-022A)

To the solution of (R)-(4-(2-(6-fluoro-1H-indol-3-yl) acetyl) morpholin-2-yl) methyl 4-methylbenzenesulfonate (800 mg, 1.79 mmol) in DMSO (20 mL) was added NaCN (96.6 mg, 1.97 mmol). The reaction was stirred at 80° C. for 3 hours and 100° C. for another 2 hours. After cooling to room temperature, the mixture was added water (40 mL), and then extracted with EtOAc (40 mL*2). The combined organic layers were washed with brine (50 mL), dried over sodium sulfate. After filtration and concentration, the residue was purified by flash column to afford desired product (400 mg, 74% yield) as a yellow solid.

MS (ESI+) m/z 302 (M+H)⁺.

### Ethyl (S)-2-(morpholin-2-yl) acetate (70-022B)

To the solution of (S)-2-(4-(2-(6-fluoro-1H-indol-3-yl) acetyl) morpholin-2-yl) acetonitrile (318 mg, 1.05 mmol) in EtOH (4 mL) at 0° C. was added SOCl₂ (0.9 mL, 12.66 mmol). After stirring at room temperature for 16 hours, the reaction mixture was concentrated to obtain ethyl (S)-2-(morpholin-2-yl)acetate (163.5 mg, 90% yield) and ethyl (S)-2-(4-(2-(6-fluoro-1H-indol-3-yl)acetyl)morpholin-2-yl)acetate as minority.

MS (ESI+) m/z 174 (M+H)⁺.

### Ethyl (S)-2-(4-(2-(6-fluoro-1H-indol-3-yl)acetyl)morpholin-2-yl)acetate (70-022) (Compound #10177)

Following general amide coupling condition I, the desired product was obtained as white solid (223 mg, 68% yield)

MS (ESI+) m/z 349 (M+H)⁺.

¹H NMR (400 MHz, CDCl₃) δ 8.16 (s, 1H), 7.60-7.49 (m, 1H), 7.11-7.00 (m, 2H), 6.96-6.84 (m, 1H), 4.63-4.50 (m, 0.5H), 4.49-4.40 (m, 0.5H), 4.23-4.04 (m, 2H), 3.97-3.59 (m, 5H), 3.55-3.45 (m, 0.5H), 3.38-3.13 (m, 1H), 2.98-2.76 (m, 1H), 2.64-2.42 (m, 2H), 2.34-2.25 (m, 0.5H), 1.31-1.20 (m, 3H).

### (S)-2-(4-(2-(6-fluoro-1H-indol-3-yl)acetyl)morpholin-2-yl)acetic acid (70-027) (Compound #10178)

The solution of ethyl (S)-2-(4-(2-(6-fluoro-1H-indol-3-yl)acetyl)morpholin-2-yl)acetate (100 mg, 0.29 mmol) in THE (8 mL) was added sodium hydroxide (8 mL, 1 N). The mixture was stirred at room temperature for 16 hours. The solvent was evaporated, and the residue was adjusted pH below 7 with HCl (10 mL, 1 N). The precipitate formed was filtered, washed with water (20 mL) and purified by Prep-HPLC to get desired product as white solid (25 mg, 27% yield).

MS (ESI+) m/z 321 (M+H)⁺.

¹H NMR (400 MHz, DMSO-d₆) δ 12.13 (br s, 1H), 10.96 (s, 1H), 7.56-7.49 (m, 1H), 7.21 (s, 1H), 7.15-7.07 (m, 1H), 6.87-6.80 (m, 1H), 4.34-4.29 (m, 0.5H), 4.18-4.14 (m, 0.5H), 4.06-4.02 (m, 0.5H), 3.90-3.70 (m, 3H), 3.62-3.52 (m, 1H), 3.33-3.19 (m, 17H), 3.11-3.02 (m, 0.5H), 2.91-2.83 (m, 0.5H), 2.72-2.62 (i, 0.5H), 2.49-2.40 (m, 2H), 2.34-2.24 (m, 1H).

### Example 42—Synthesis of Further Compounds Using the Procedure of Example 41

Using the above procedures, the following compounds were synthesized:

TABLE 14

| | Compound | | LC-MS |
|---|---|---|---|
| Structure | # | ¹H NMR | (M + H)⁺ |
| 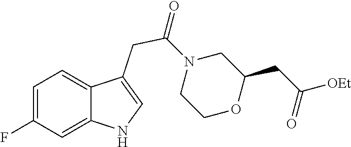 [SMILES: CCOC(=O)C[C@@H]1CN(C(=O)Cc2c[nH]c3cc(F)ccc23)CCO1] | 10171 | ¹H NMR (400 MHz, CDCl₃) δ 8.14 (s, 1H), 7.60-7.48 (m, 1H), 7.12-7.00 (m, 2H), 6.95- 6.84 (m, 1H), 4.58-4.38 (m, 1H), 4.21- 4.05 (m, 2H), 4.03-3.57 (m, 5H), 3.56-3.44 (m, 0.5H), 3.38-3.26 (m, 0.5H), 3.25-3.13 (m, 0.5H), 2.98-2.76 (m, 1H), 2.64-2.40 (m, 2H), 2.35-2.21 (m, 0.5H), 1.31-1.19 (m, 3H). | 349 |
| 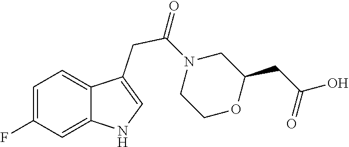 [SMILES: O=C(O)C[C@@H]1CN(C(=O)Cc2c[nH]c3cc(F)ccc23)CCO1] | 10175 | ¹H NMR (400 MHz, DMSO-d₆) δ 12.30 (br s, 1H), 10.97 (s, 1H), 7.56-7.49 (m, 1H), 7.24- 7.19 (m, 1H), 7.15-7.07 (m, 1H), 6.88-6.79 (m, 1H), 4.32 (d, J = 13.1 Hz, 0.5H), 4.16 (d, J = 13.0 Hz, 0.5H), 4.10-4.00 (m, 0.5H), 3.87 (d, J = 12.9 Hz, 0.5H), 3.83-3.68 (m, 3H), 3.65-3.51 (m, 1H), 3.34-3.18 (m, 1.5H), 3.13-3.00 (m, 0.5H), 2.92-2.82 (m, 0.5H), 2.73-2.61 (m, 0.5H), 2.49-2.37 (m, 2H), 2.35-2.22 (m, 1H). | 321 |

### Example 43—Synthesis of (R)-1-(2-(6-fluoro-1H-indol-3-yl) acetyl)-4-methylpiperazine-2-carboxylic Acid (Compound #10165) (70-006 in Scheme 29)

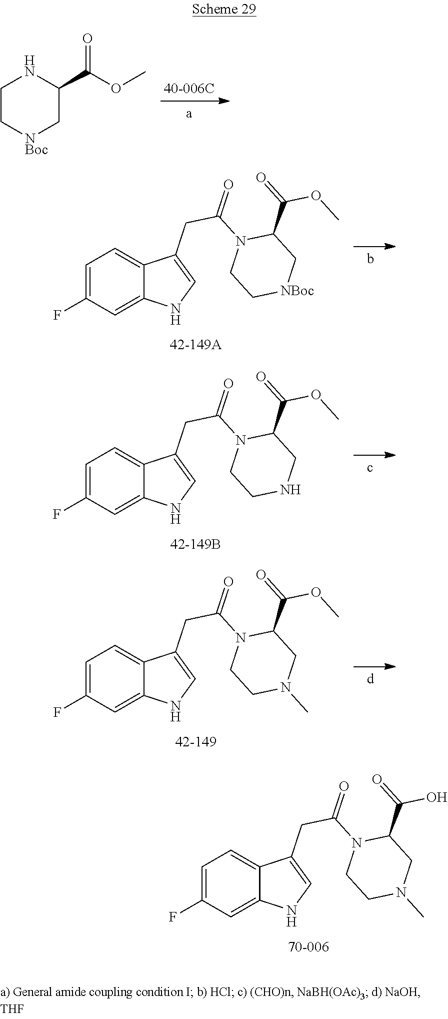 [SMILES: COC(=O)[C@H]1CN(C(=O)OC(C)(C)C)CCN1 ; COC(=O)[C@H]1CN(C(=O)OC(C)(C)C)CCN1C(=O)Cc1c[nH]c2cc(F)ccc12 ; COC(=O)[C@H]1CNCCN1C(=O)Cc1c[nH]c2cc(F)ccc12 ; COC(=O)[C@H]1CN(C)CCN1C(=O)Cc1c[nH]c2cc(F)ccc12 ; CN1CCN(C(=O)Cc2c[nH]c3cc(F)ccc23)[C@@H](C(=O)O)C1]

### 1-(tert-butyl) 3-methyl (R)-4-(2-(6-fluoro-1H-indol-3-yl) acetyl) piperazine-1,3-Dicarboxylate (42-149A)

Following general amide coupling condition I, the desired product was obtained as yellow oil (1.8 g, 90% yield).

MS (ESI+) m/z 364 (M+H)⁺.

### Methyl (R)-1-(2-(6-fluoro-1H-indol-3-yl) acetyl) piperazine-2-carboxylate (42-149B)

The solution of 1-(tert-butyl) 3-methyl (R)-4-(2-(6-fluoro-1H-indol-3-yl) acetyl) piperazine-1,3-dicarboxylate (1.8 g, 4.29 mmol) in HCl (16 mL, 4.0 M in methanol) was stirred at room temperature for 4 hours. Then, the mixture was concentrated to get crude product for the next step use without further purification.

MS (ESI+) m/z 320 (M+H)⁺.

### Methyl (R)-1-(2-(6-fluoro-1H-indol-3-yl) acetyl)-4-methylpiperazine-2-carboxylate (42-149) (Compound #10157)

The solution of (2R)-1-[2-(6-fluoro-1H-indol-3-yl) acetyl] piperazine-2-carboxylate (1.24 g, 3.88 mmol) and Et₃N (0.39 g, 3.88 mmol) in MeOH (20 mL) was stirred at room temperature for 20 minutes. Then, formaldehyde (0.583 g, 19.42 mmol), AcOH (45.74 mg, 0.78 mmol) and NaBH(OAc)₃ (4.10 g, 19.40 mmol) were added to above solution. The resulting system was continued to stir at room temperature for 16 hours. After which period, saturated NaHCO₃ (60 mL) was added to the reaction and the mixture was extracted with DCM (100 mL). The separated organic phase was dried over sodium sulfate, filtered, concentrated to dryness, which was purified by Prep-HPLC to afford desired product as white solid (800 mg, 62% yield).

MS (ESI+) m/z 334 (M+H)⁺.

¹H NMR (400 MHz, DMSO-d₆) δ 11.04-10.88 (m, 1H), 7.58-7.42 (m, 1H), 7.26-7.07 (m, 2H), 6.89-6.77 (m, 1H), 5.06-4.92 (m, 1H), 4.22-4.13 (m, 0.3H), 3.94-3.68 (m, 3H), 3.61 (s, 2H), 3.49 (s, 1H), 3.31-3.08 (m, 1.7H), 2.80-2.62 (m, 1H), 2.12 (s, 3H), 2.08-1.97 (m, 1H), 1.84-1.69 (m, 1H).

### (R)-1-(2-(6-fluoro-1H-indol-3-yl) acetyl)-4-methylpiperazine-2-carboxylic acid (70-006) (Compound #10165)

To the solution of methyl (R)-1-(2-(6-fluoro-1H-indol-3-yl) acetyl)-4-methylpiperazine-2-carboxylate (653 mg, 1.96 mmol) in THE (10 mL) was added sodium hydroxide (10 mL, 1.0 N). The mixture was stirred at room temperature for 16 hours, then the solvent was evaporated. The residue was adjusted pH below 7 with HCl (14 mL, 1N). The precipitate formed was filtered and washed with water (14 mL), which was further purified by prep-HPLC to get desired product as white solid (450 mg, 71% yield).

MS (ESI+) m/z 320 (M+H)⁺.

¹H NMR (400 MHz, DMSO-d₆) δ 11.13-10.97 (m, 1H), 7.56-7.43 (m, 1H), 7.26 (s, 1H), 7.20-7.07 (m, 1H), 6.95-6.72 (m, 1H), 5.33 (s, 1H), 4.52-4.43 (m, 0.5H), 4.23-4.15 (m, 0.5H), 3.93-3.69 (m, 3H), 3.47-3.06 (m, 3H), 2.96-2.75 (m, 4H).

### Example 44—Synthesis of Further Compounds Using the Procedure of Example 43

Using the above procedures, the following examples were synthesized:

TABLE 15

| | Compound | | LC-MS |
|---|---|---|---|
| Structure | # | ¹H NMR | (M + H)⁺ |
| 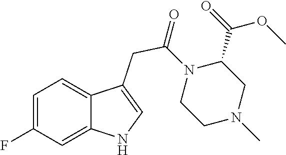 [SMILES: COC(=O)[C@@H]1CN(C)CCN1C(=O)Cc1c[nH]c2cc(F)ccc12] | 10167 | ¹H NMR (400 MHz, DMSO-d₆) δ 11.01-10.89 (m, 1H), 7.52-7.45 (m, 1H), 7.24-7.07 (m, 2H), 6.89- 6.79 (m, 1H), 5.08-4.93 (m, 1H), 4.22-4.13 (m, 0.3H), 3.92-3.65 (m, 3H), 3.61 (s, 2H), 3.49 (s, 1H), 3.31-3.14 (m, 1.7H), 2.79-2.63 (m, 1H), 2.12 (s, 3H), 2.06-1.96 (m, 1H), 1.83-1.70 (m, 1H).. | 334 |
| 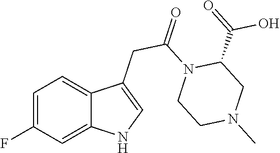 [SMILES: CN1CCN(C(=O)Cc2c[nH]c3cc(F)ccc23)[C@H](C(=O)O)C1] | 10158 | ¹H NMR (400 MHz, DMSO-d₆) δ 11.11-10.94 (m, 1H), 7.53-7.42 (m, 1H), 7.25 (s, 1H), 7.17-7.08 (m, 1H), 6.93-6.72 (m, 1H), 5.31 (s, 1H), 4.50-4.42 (m, 0.4H), 4.22-4.14 (m, 0.6H), 3.91-3.68 (m, 3H), 3.45-3.04 (m, 3H), 2.91-2.72 (m, 4H). | 320 |

### Example 45—Synthesis of 2-(6-fluoro-1H-indol-3-yl)-1-(4-(tetrahydrofuran-2-carbonyl)piperazin-1-yl)ethan-1-one (Compound #10170) (41-139 in Scheme 30)

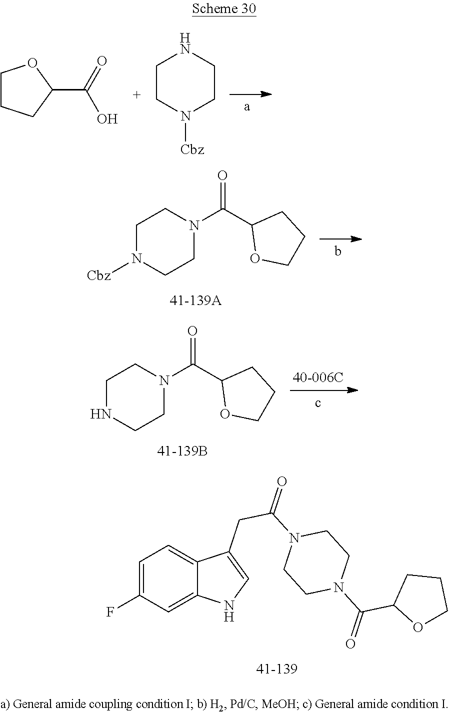

### Benzyl 4-(tetrahydrofuran-2-carbonyl)piperazine-1-carboxylate (41-139A)

Following general amide coupling condition I, the desired product was obtained from flash column chromatography (elute with EtOAc:PE=0:1 to 1:0) (408 mg, 54.2% yield) as colorless oil.

MS (ESI+) m/z 319 (M+H)⁺.

### Piperazin-1-yl(tetrahydrofuran-2-yl)methanone (41-139B)

The solution of benzyl 4-(oxolane-2-carbonyl) piperazine-1-carboxylate (0.41 g, 1.28 mmol) and Pd/C (218 mg, 2.04 mmol) in MeOH (15 mL) was stirred under H₂ at room temperature for 12 hours. The mixture was filtered through celite and concentrated under vacuum to get piperazin-1-yl(tetrahydrofuran-2-yl)methanone (230 mg, 97.4% yield) as colorless oil.

MS (ESI+) m/z 185 (M+H)⁺.

### 2-(6-fluoro-1H-indol-3-yl)-1-(4-(tetrahydrofuran-2-carbonyl)piperazin-1-yl)ethan-1-one (41-139) Compound #10170

Following general amide coupling condition I, the desired product was obtained as white solid (68 mg, 36.5% yield).

MS (ESI+) m/z 360 (M+H)⁺.

¹H NMR (400 MHz, CDCl₃) δ 8.24 (s, 1H), 7.61-7.49 (m, 1H), 7.09-7.00 (m, 2H), 6.95-6.85 (m, 1H), 4.60-4.44 (m, 1H), 3.96-3.33 (m, 11H), 3.30-3.16 (m, 1H), 2.39-2.20 (m, 1H), 2.10-1.82 (m, 3H).

### Example 46—Synthesis of (R)-4-(2-(6-fluoro-1H-indol-3-yl)acetyl)-1-methylpiperazine-2-carboxylic acid (Compound #10166) (69-003 in Scheme 31)

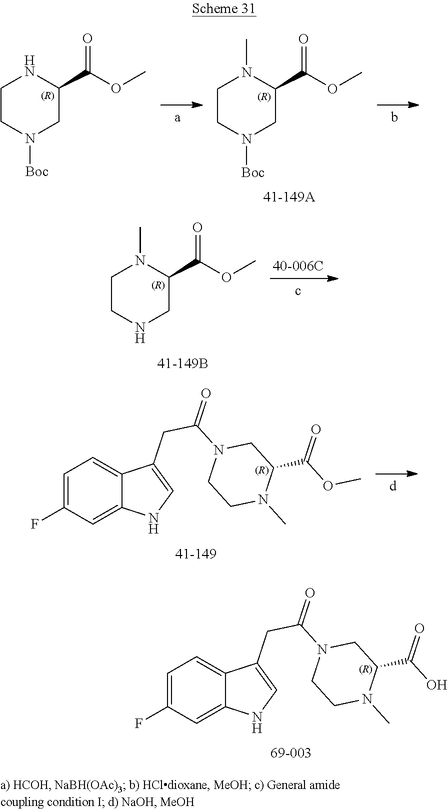

### 1-(tert-butyl) 3-methyl (R)-4-methylpiperazine-1,3-dicarboxylate (41-149A)

To the solution of (R)-tert-butyl methyl piperazine-1,3-dicarboxylate (1.50 g, 6.14 mmol) in MeCN and MeOH (1:1, 120 mL) was added formaldehyde (37 percent aqueous, 16.5 mL, 221 mmol), followed by the addition of sodium triacetoxyborohydride (6.5 g, 30.66 mmol). The mixture was stirred for about 15 minutes at room temperature. AcOH (7.0 mL, 122.5 mmol) was added dropwise and the mixture was stirred for about 1 h. The solvent was removed under reduced pressure and the residue was dissolved in DCM (100 mL) and neutralized using aqueous 2 N NaOH. Saturated aqueous NaHCO₃ (50 mL) was added and the layers were separated. The organic layer was washed with brine (50 mL), dried over anhydrous Na₂SO₄, filtered, and concentrated under reduced pressure. The crude material was purified by silica gel chromatography eluting with a gradient of 20-80 percent EtOAc/heptane to afford (R)-1-tert-butyl 3-methyl 4-methylpiperazine-1,3-dicarboxylate (1.2 g, 75.7% yield).

MS (ESI+) m/z 259 (M+H)⁺.

### Methyl (R)-1-methylpiperazine-2-carboxylate (41-149B)

The mixture of (R)-tert-butyl methyl 4-methylpiperazine-1,3-dicarboxylate (1.20 g, 4.64 mmol) in HCl/dioxane (6N, 15 mL) and MeOH (15 mL) was stirred for 2 hours at room temperature. Then, the mixture was concentrated to get crude product for the next step without further purification.

MS (ESI+) m/z 195 (M+H)⁺.

### Methyl (R)-4-(2-(6-fluoro-1H-indol-3-yl)acetyl)-1-methylpiperazine-2-carboxylate (41-149)(Compound #10156)

Following general amide coupling condition I, the desired product was obtained (180 mg, 52.2% yield) as white solid.

MS (ESI+) m/z 334 (M+H)⁺.

¹H NMR (400 MHz, CDCl₃) δ 8.18 (s, 1H), 7.59-7.47 (m, 1H), 7.09-7.00 (m, 2H), 6.94-6.85 (m, 1H), 4.24-4.15 (m, 0.5H), 4.12-4.01 (m, 0.5H), 3.90-3.76 (m, 2.5H), 3.76-3.59 (m, 3.5H), 3.56-3.41 (m, 1.5H), 3.40-3.27 (m, 0.5H), 3.02-2.85 (m, 1.5H), 2.77-2.66 (m, 0.5H), 2.37-2.25 (m, 3H), 2.25-2.15 (m, 0.5H), 2.14-2.02 (m, 0.5H).

### (R)-4-(2-(6-fluoro-1H-indol-3-yl)acetyl)-1-methylpiperazine-2-carboxylic Acid (69-003) Compound #10166

Methyl 4-[2-(6-fluoro-1H-indol-3-yl)acetyl]-1-methyl-piperazine-2-carboxylate (0.1 g, 0.30 mmol) was dissolved in MeOH/H₂O=10:1 (5.5 mLQ, then NaOH (1 M, 1 mLQ was added dropwise, and the mixture was stirred at room temperature for 12 hours. HCl (1N) was added dropwise till the pH=3, then the precipitate was filtered and washed with water (5 mL) to obtain (R)-4-(2-(6-fluoro-1H-indol-3-yl)acetyl)-1-methylpiperazine-2-carboxylic acid (86 mg, 89.8% yield) as white solid.

MS (ESI+) m/z 320 (M+H)+.

¹H NMR (400 MHz, DMSO-d₆) δ 11.01 (s, 1H), 7.58-7.43 (m, 17H), 7.26-7.18 (m, 16H), 7.15-7.09 (m, 1H), 6.92-6.77 (m, 1H), 4.41-4.28 (m, 0.5H), 4.18-3.93 (m, 1H), 3.92-3.73 (m, 2.5H), 3.72-3.58 (m, 0.5H), 3.55-3.41 (m, 0.5H), 3.39-3.19 (m, 3H), 2.96-2.78 (m, 10H), 2.78-2.62 (m, 3H).

### Example 47—Synthesis of Further Compounds Using the Procedure of Example 46

Using the above procedures, the following compounds were synthesized:

TABLE 16

| | Compound | | LC-MS |
|---|---|---|---|
| Structure | # | ¹H NMR | (M + H)⁺ |
| 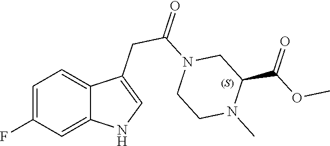 | 10163 | ¹H NMR (400 MHz, CDCl₃) δ 8.16 (s, 1H), 7.58- 7.48 (m, 1H), 7.09-7.00 (m, 2H), 6.95-6.86 (m, 1H), 4.24-4.13 (m, 0.5H), 4.12-4.01 (m, 0.5H), 3.89-3.76 (m, 2.5H), 3.75-3.60 (m, 3.5H), 3.57-3.41 (m, 1.5H), 3.40-3.28 (m, 0.5H), 3.05-2.85 (m, 1.5H), 2.78-2.63 (m, 0.5H), 2.42-2.02 (m, 4H). | 334 |
| 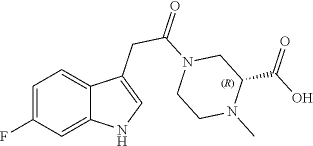 | 10173 | ¹H NMR (400 MHz, DMSO-d₆) δ 11.02 (s, 1H), 7.59-7.42 (m, 1H), 7.27-7.18 (m, 1H), 7.17- 7.07 (m, 1H), 6.91-6.77 (m, 1H), 4.42-4.29 (m, 0.5H), 4.18-3.94 (m, 1H), 3.93-3.73 (m, 2.5H), 3.73-3.57 (m, 0.5H), 3.55-3.41 (m, 0.5H), 3.37-3.16 (m, 3H), 2.95-2.77 (m, 1H), 2.77-2.66 (m, 3H). | 320 |

### Example 48—Synthesis of (R)—N-(1-acetylpyrrolidin-3-yl)-2-(6-fluoro-1H-indol-3-yl) acetamide (Compound #10092) (42-067 in Scheme 32)

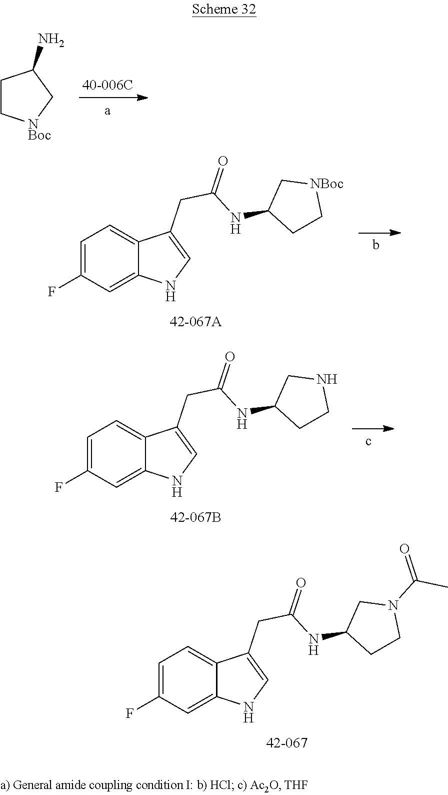 [SMILES: CC(C)(C)OC(=O)N1CC[C@@H](N)C1 ; CC(C)(C)OC(=O)N1CC[C@@H](NC(=O)Cc2c[nH]c3cc(F)ccc23)C1 ; O=C(Cc1c[nH]c2cc(F)ccc12)N[C@@H]1CCNC1 ; CC(=O)N1CC[C@@H](NC(=O)Cc2c[nH]c3cc(F)ccc23)C1]

### tert-butyl (R)-3-(2-(6-fluoro-1H-indol-3-yl)acetamido)pyrrolidine-1-carboxylate (42-067A)

Following general amide coupling condition I, the desired product was obtained as yellow oil (550 mg, 98% yield).

MS (ESI+) m/z 306 (M+H)⁺.

### (R)-2-(6-fluoro-1H-indol-3-yl)-N-(pyrrolidin-3-yl)acetamide (42-067B)

The solution of tert-butyl-(R)-3-(2-(6-fluoro-1H-indol-3-yl)acetamido)pyrrolidine-1-carboxylate (550 mg, 1.52 mmol) in HCl (10 mL, 4.0 M in methanol) was stirred at room temperature for 4 hours. Then, the mixture was concentrated to get crude product as yellow solid (318 mg, 80% yield) for the next step without further purification.

MS (ESI+) m/z 262 (M+H)⁺.

### (R)—N-(1-acetylpyrrolidin-3-yl)-2-(6-fluoro-1H-indol-3-yl) acetamide (42-067) Compound #10092

Ac₂O (43.0 mg, 0.42 mmol) and Et₃N (115.1 mg, 1.14 mmol) were added to a stirred solution of (R)-2-(6-fluoro-1H-indol-3-yl)-N-(yrrolidinedin-3-yl)acetamide (100 mg, 0.38 mmol) in THE (8 mL). The resulting reaction mixture was stirred at room temperature for 3 hours. After which period, the mixture was diluted with EA (30 mL), washed with brine (30 mL), dried over sodium sulfate. After filtration and concentration to dryness, the residue was purify by Prep-HPLC to obtain desired product as white solid (50 mg, 43% yield)

MS (ESI+) m/z 304 (M+H)⁺.

¹H NMR (400 MHz, CDCl₃) δ 8.30 (s, 1H), 7.48-7.37 (m, 1H), 7.17-7.05 (m, 2H), 6.99-6.87 (m, 1H), 5.78-5.68 (m, 0.5H), 5.66-5.59 (m, 0.5H), 4.57-4.39 (m, 1H), 3.70 (d, J=3.7 Hz, 2.5H), 3.64-3.55 (m, 0.5H), 3.48-3.21 (m, 2H), 3.18-3.08 (m, 1H), 2.22-2.01 (m, 1H), 1.98-1.92 (m, 3H), 1.90-1.80 (m, 0.5H), 1.61-1.56 (m, 0.5H).

### Example 49—Synthesis of Further Compounds Using the Procedure of Example 48

Using the above procedures, the following examples were synthesized:

TABLE 17

| | Compound | | LC-MS |
|---|---|---|---|
| Structure | # | ¹H NMR | (M + H)⁺ |
| 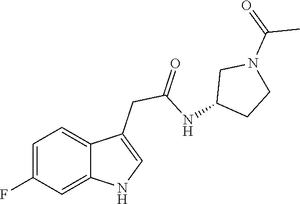 [SMILES: CC(=O)N1CC[C@H](NC(=O)Cc2c[nH]c3cc(F)ccc23)C1] | 10090 | ¹H NMR (400 MHz, CDCl₃) δ 8.58 (s, 1H), 7.48- 7.39 (m, 1H), 7.14-7.04 (m, 2H), 6.97-6.87 (m, 1H), 5.89-5.80 (m, 0.5H), 5.77-5.73 (m, 0.5H), 4.54-4.38 (m, 1H), 3.74-3.66 (m, 2.5H), 3.63- 3.54 (m, 0.5H), 3.48-3.23 (m, 2H), 3.19-3.12 (m, 1H), 2.23-2.00 (m, 1H), 1.94 (d, J = 5.7 Hz, 3H), 1.89-1.77 (m, 0.5H), 1.67-1.56 (m, 0.5H). | 304 |

### Example 50—Synthesis of Compound #10087, 10113 and 10114 (42-078, 40-096, and 42-098 in Scheme 33, respectively)

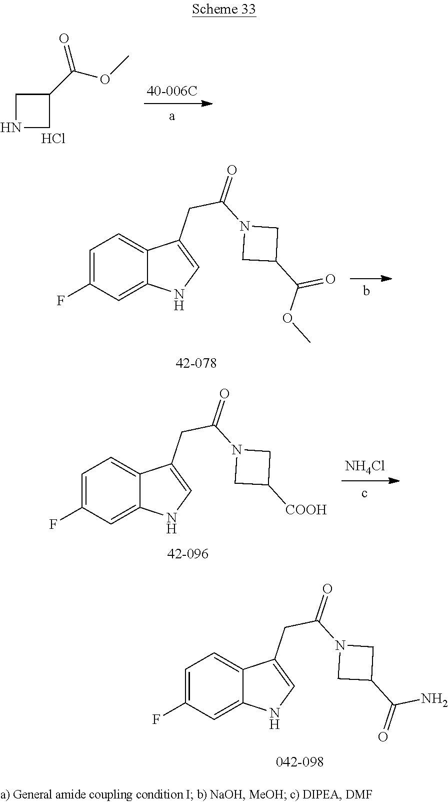

### methyl 1-(2-(6-fluoro-1H-indol-3-yl) acetyl) azetidine-3-carboxylate (42-078) Compound #10087

Following general amide coupling condition I, the desired product was obtained as white solid (2.5 g, 82% yield).

MS (ESI+) m/z 291 (M+H)⁺.

¹H NMR (400 MHz, CDCl₃) δ 8.23 (s, 1H), 7.55-7.50 (m, 1H), 7.10-7.06 (m, 1H), 7.03-6.99 (m, 1H), 6.94-6.85 (m, 1H), 4.34-4.15 (m, 4H), 3.74 (s, 3H), 3.61-3.56 (m, 2H), 3.42-3.32 (m, 1H).

### 1-(2-(6-fluoro-1H-indol-3-yl) acetyl) azetidine-3-carboxylic acid (42-096) Compound #10113

The solution of methyl 1-(2-(6-fluoro-1H-indol-3-yl)acetyl)azetidine-3-carboxylate (2.46 g, 8.48 mmol) in methanol (20 mL) was added sodium hydroxide (20 mL, 1 N). Then, the mixture was stirred at room temperature for 16 hours. Then, the solvent was evaporated. The residue was treated with HCl (25 mL, 1N) to adjust pH below 7. The aqueous mixture was extracted with EtOAc (3×40 mL), and the combined organic layers were dried over sodium sulfate and concentrated to dryness. The resulting residue was purified by Prep-HPLC to get desired product as white solid (2 g, 85% yield).

MS (ESI+) m/z 277 (M+H)⁺.

¹H NMR (400 MHz, DMSO-d₆) δ 12.64 (br s, 1H), 10.97 (s, 1H), 7.54-7.47 (m, 1H), 7.24-7.19 (m, 1H), 7.14-7.08 (m, 1H), 6.88-6.79 (m, 1H), 4.30 (t, J=8.8 Hz, 1H), 4.25-4.17 (m, 1H), 4.00 (t, J=9.3 Hz, 1H), 3.89-3.82 (m, 1H), 3.54-3.44 (m, 2H), 3.41-3.34 (m, 1H).

### 1-(2-(6-fluoro-1H-indol-3-yl)acetyl)azetidine-3-carboxamide (42-098) Compound #10114

The solution of 1-(2-(6-fluoro-1H-indol-3-yl)acetyl)azetidine-3-carboxylic acid (400 mg, 1.45 mmol), NH₄Cl (716 mg, 14.5 mmol), DIPEA (936 mg, 7.24 mmol) and HATU (826 mg, 2.17 mmol) in DMF (10 mL) was stirred at room temperature for 16 hours. Then, the mixture was diluted with brine (40 mL) and extracted with EtOAc (30 mL * 3). The combined organic layers were dried over sodium sulfate and concentrated to dryness. The residue was purified by Prep-HPLC to get desired product as white solid (53.2 mg, 13% yield).

MS (ESI+) m/z 276 (M+H)⁺.

¹H NMR (400 MHz, DMSO-d₆) δ 10.96 (s, 1H), 7.53-7.48 (m, 1H), 7.45 (s, 1H), 7.23-7.18 (m, 1H), 7.13-7.08 (m, 1H), 7.03 (s, 1H), 6.87-6.80 (m, 1H), 4.26-4.19 (m, 1H), 4.18-4.13 (m, 1H), 3.95-3.87 (m, 1H), 3.85-3.78 (m, 1H), 3.47 (s, 2H), 3.29-3.20 (m, 1H).

### Example 51—Synthesis of 1-(3-(cyclopropanecarbonyl)azetidin-1-yl)-2-(6-fluoro-1H-indol-3-yl)ethan-1-one (Compound #10115) (42-101 in Scheme 34)

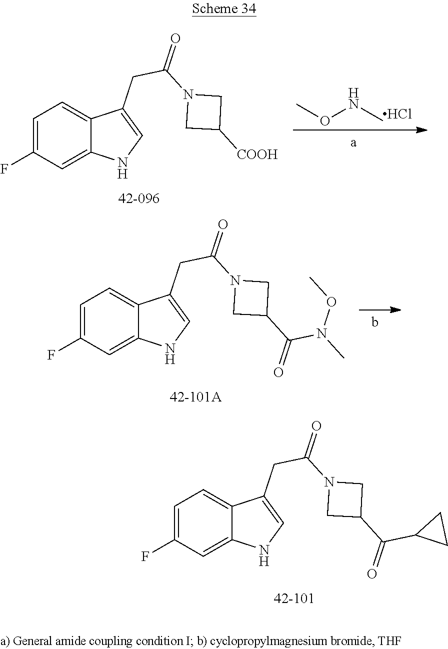

### 1-(2-(6-fluoro-1H-indol-3-yl)acetyl)-N-methoxy-N-methylazetidine-3-carboxamide (42-101A)

Following general amide coupling condition I, the desired product was obtained as yellow solid (1 g, 87% yield).

MS (ESI+) m/z 320 (M+H)⁺.

¹H NMR (400 MHz, DMSO-d₆) δ 10.96 (s, 1H), 7.53-7.48 (m, 1H), 7.21-7.20 (m, 1H), 7.13-7.09 (m, 1H), 6.87-6.79 (m, 1H), 4.32-4.20 (m, 2H), 4.02-3.94 (m, 1H), 3.92-3.85 (m, 1H), 3.68 (s, 1H), 3.63 (s, 3H), 3.49 (s, 2H), 3.11 (s, 3H).

### 1-(3-(cyclopropanecarbonyl)azetidin-1-yl)-2-(6-fluoro-1H-indol-3-yl)ethan-1-one (42-101) Compound #10115

To a stirred solution of 1-(2-(6-fluoro-1H-indol-3-yl)acetyl)-N-methoxy-N-methylazetidine-3-carboxamide (100 mg, 0.31 mmol) in THE (5 mL) was added cyclopropyl magnesium bromide (1.0 mL, 0.93 mmol, 1 M in THF) dropwise at 0° C. After being stirred at 0° C. for 2 hours under argon atmosphere, the reaction was quenched with aqueous saturated NH₄Cl solution (15 mL). The resulting mixture was extracted with EtOAc (50 mL), and the combined organic layers were washed with brine (30 mL), dried over sodium sulfate. After filtration and concentration to dryness, the residue was purified by Prep-HPLC to obtain title compound as white solid (50.3 mg, 53% yield).

MS (ESI+) m/z 301 (M+H)⁺.

¹H NMR (400 MHz, DMSO-d₆) δ 10.97 (s, 1H), 7.53-7.46 (m, 1H), 7.21-7.18 (m, 1H), 7.14-7.08 (m, 1H), 6.87-6.79 (m, 1H), 4.33-4.19 (m, 2H), 4.01 (t, J=9.3 Hz, 1H), 3.92-3.84 (m, 1H), 3.77-3.68 (m, 1H), 3.54-3.43 (m, 2H), 2.04-1.95 (m, 1H), 0.96-0.84 (m, 4H).

### Example 52—Synthesis of Further Compounds Using the Procedure of Example 51

Using the above procedures, the following examples were synthesized:

TABLE 18

| | Compound | | LC-MS |
|---|---|---|---|
| Structure | # | ¹H NMR | (M + H)⁺ |
| 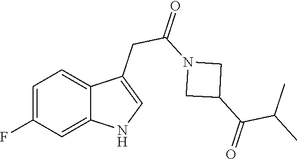 [SMILES: CC(C)C(=O)C1CN(C(=O)Cc2c[nH]c3cc(F)ccc23)C1] | 10116 | ¹H NMR (400 MHz, DMSO-d₆) δ 10.96 (s, 1H), 7.54- 7.46 (m, 1H), 7.24-7.18 (m, 1H), 7.15-7.07 (m, 1H), 6.91-6.79 (m, 1H), 4.28-4.15 (m, 2H), 3.99- 3.90 (m, 1H), 3.88-3.74 (m, 2H), 3.52-3.42 (m, 2H), 2.70-2.60 (m, 1H), 1.03-0.97 (m, 6H). | 303 |
| 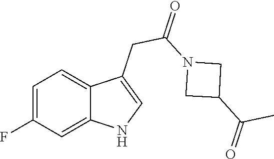 [SMILES: CC(=O)C1CN(C(=O)Cc2c[nH]c3cc(F)ccc23)C1] | 10138 | ¹H NMR (400 MHz, DMSO-d₆) δ 10.96 (s, 1H), 7.53- 7.46 (m, 1H), 7.21-7.18 (m, 1H), 7.14-7.08 (m, 1H), 6.87-6.79 (m, 1H), 4.25-4.20 (m, 2H), 4.00- 3.84 (m, 2H), 3.64-3.50 (m, 1H), 3.50-3.45 (m, 2H), 2.12 (s, 3H). | 275 |

### Example 53—Synthesis of 2-(6-fluoro-1H-indol-3-yl)-1-(3-hydroxy-3-(methoxymethyl)azetidin-1-yl)ethan-1-one (Compound #10089) (40-079 in Scheme 35)

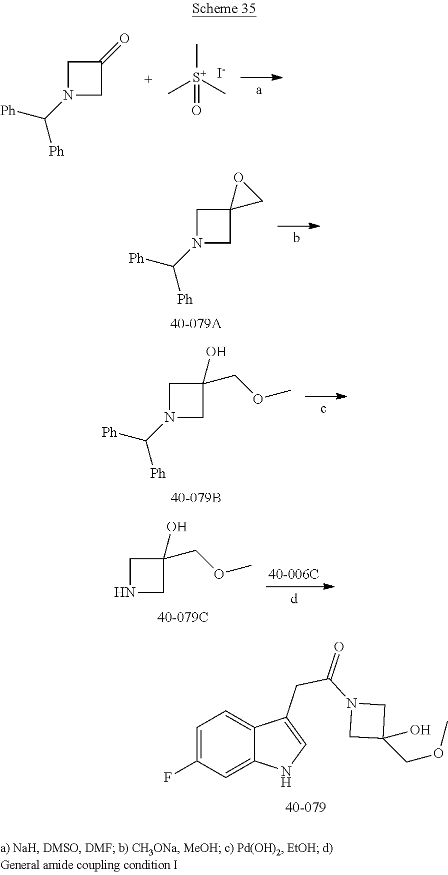

### 5-benzhydryl-1-oxa-5-azaspiro[2.3]hexane (40-079A)

A mixture of trimethylsulphoxonium iodide (5.6 g, 25.28 mmol), sodium hydride (1 g, 25.28 mmol) and dimethylformamide (120 mL) was cooled to 4° C., and treated with DMSO (1.8 mL, 25.28 mmol). After stirring at 4° C. for 20 minutes, a solution of 1-diphenylmethyl-3-azetidinone (6 g, 25.28 mmol) in dimethylformamide (60 mL) was added dropwise. The reaction mixture was stirred for 30 minutes at 4° C., then quenched with water. The aqueous was extracted twice with ethyl acetate (120 mL*2). The combined organics were washed with water (3 times), dried over sodium sulfate, then evaporated to dryness. The residue was purified by flash column to get 5-benzhydryl-1-oxa-5-azaspiro[2.3]hexane as yellow oil (1.1 g, 17.3% yield).

MS (ESI+) m/z 252 (M+H)⁺.

### 1-benzhydryl-3-(methoxymethyl) azetidin-3-ol (40-079B)

To the solution of 5-benzhydryl-1-oxa-5-azaspiro [2.3] hexane (1.0 g, 3.40 mmol) in methanol (10 mL) was added sodium methanolate (10 mL, 5.0 N) dropwise. Then, the mixture was stirred at room temperature for 2 hours and evaporated in vacuum. The residue was taken up in aqueous ammonium chloride and extracted with ethyl acetate. The organic layer was dried over sodium sulfate, filtered and concentrated to dryness. The residue was purified by flash column to obtain title compound (1.0 g, 88.7% yield).

MS (ESI+) m/z 284 (M+H)⁺.

¹H NMR (400 MHz, CDCl₃) δ 7.49-7.36 (m, 4H), 7.27-7.24 (m, 4H), 7.21-7.11 (m, 2H), 4.39 (s, 1H), 3.65 (s, 2H), 3.43 (s, 3H), 3.29-3.27 (m, 2H), 2.97-2.95 (m, 2H).

### 3-(methoxymethyl) azetidin-3-ol (40-079C)

The solution of 1-benzhydryl-3-(methoxymethyl) azetidin-3-ol (500 mg, 1.76 mmol) and Pd(OH)₂ (500 mg) in ethanol (15 mL) was stirred at 60° C. under H₂ for 3 hours. After cooling to room temperature, the mixture was filtered with celite. The filtrate was concentrated to get crude product (500 mg, brown gel) for the next step without further purification.

MS (ESI+) m/z 118 (M+H)⁺.

### 2-(6-fluoro-1H-indol-3-yl)-1-(3-hydroxy-3-(methoxymethyl)azetidin-1-yl)ethan-1-one (40-079)

Following general amide coupling condition I, the desired product was obtained as white solid (36.0 mg, 23.8% yield).

MS (ESI+) m/z 293 (M+H)⁺.

¹H NMR (400 MHz, CDCl₃) δ 8.10 (s, 1H), 7.54-7.51 (m, 1H), 7.17-7.07 (m, 1H), 7.04-7.02 (m, 1H), 6.95-6.84 (m, 1H), 4.05-3.89 (m, 4H), 3.59 (s, 2H), 3.48 (s, 2H), 3.41 (s, 3H), 2.90 (s, 1H).

### Example 54—Synthesis of 2-(6-fluoro-1H-indol-3-yl)-1-(3-(methoxymethyl)-3-methylazetidin-1-yl)ethan-1-one (Compound #10134) (42-120 in Scheme 36)

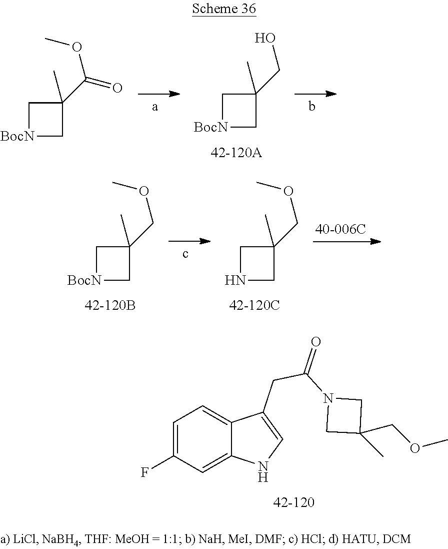

### tert-Butyl 3-(hydroxymethyl)-3-methylazetidine-1-carboxylate (42-120A)

The solution of 1-(tert-butyl) 3-methyl 3-methylazetidine-1,3-dicarboxylate (900 mg, 3.93 mmol), LiCl (366.1 mg, 8.64 mmol) and NaBH₄ (371.3 mg, 9.81 mmol) in THE (10 mL) and MeOH (10 mL) was stirred at room temperature for 16 hours. Then, the mixture was diluted with EtOAc (50 mL), and washed with a saturated ammonium chloride aqueous solution (50 mL). The separated organic phase was dried over sodium sulfate and concentrated to dryness. The residue was purified by flash column to obtain desired product as yellow oil (690 mg, 87% yield)

¹H NMR (400 MHz, DMSO-d₆) δ 3.79-3.75 (m, 2H), 3.61 (s, 2H), 3.57-3.53 (m, 2H), 1.45 (s, 9H), 1.27 (s, 3H).

### tert-Butyl 3-(methoxymethyl)-3-methylazetidine-1-carboxylate (42-120B)

To a stirred solution of tert-butyl 3-(hydroxymethyl)-3-methylazetidine-1-carboxylate (640 mg, 3.18 mmol) in DMF (10 mL) was added NaH (254.4 mg, 6.36 mmol) by portions at 0° C. and the mixture was stirred for 20 minutes. Then, iodomethane (677 mg, 4.77 mmol) was added dropwise and the resulting mixture was slowly warmed to room temperature and stirred for 16 hours. After which period, the reaction was quenched by water (50 mL), extracted with EtOAc (60 mL). The separated organic phase was washed with brine (50 mL), dried over sodium sulfate, filtered and concentrated to dryness. The residue was purified by flash column to obtain desired product as yellow oil (600 mg, 88% yield).

¹H NMR (400 MHz, CDCl₃) δ 3.76-3.72 (m, 2H), 3.56-3.51 (m, 2H), 3.38 (s, 3H), 3.33 (s, 2H), 2.05 (s, 3H), 1.44 (s, 9H).

### 3-(methoxymethyl)-3-methylazetidine (42-120C)

The solution of tert-butyl 3-(methoxymethyl)-3-methylazetidine-1-carboxylate (479 mg, 2.22 mmol) in HCl (8 mL, 4.0 M in methanol) was stirred at room temperature for 4 hours. Then, the mixture was concentrated to get crude product for the next step without further purification.

MS (ESI+) m/z 116 (M+H)⁺.

### 2-(6-fluoro-1H-indol-3-yl)-1-(3-(methoxymethyl)-3-methylazetidin-1-yl)ethan-1-one (42-120) (Compound #10134)

Following the general amide coupling condition I, the desired product was obtained as white solid (60.8 mg, 61% yield).

MS (ESI+) m/z 291 (M+H)⁺. 291

¹H NMR (400 MHz, DMSO-d₆) δ 10.94 (s, 1H), 7.55-7.43 (m, 1H), 7.20-7.09 (m, 2H), 6.87-6.78 (m, 1H), 3.99-3.93 (m, 1H), 3.76-3.72 (m, 1H), 3.67-3.62 (m, 1H), 3.47-3.41 (m, 3H), 3.29-3.26 (m, 5H), 1.17 (s, 3H).

### Example 55—Synthesis of Compounds #10088 and 10102 (41-080 and 41-084 in Scheme 37)

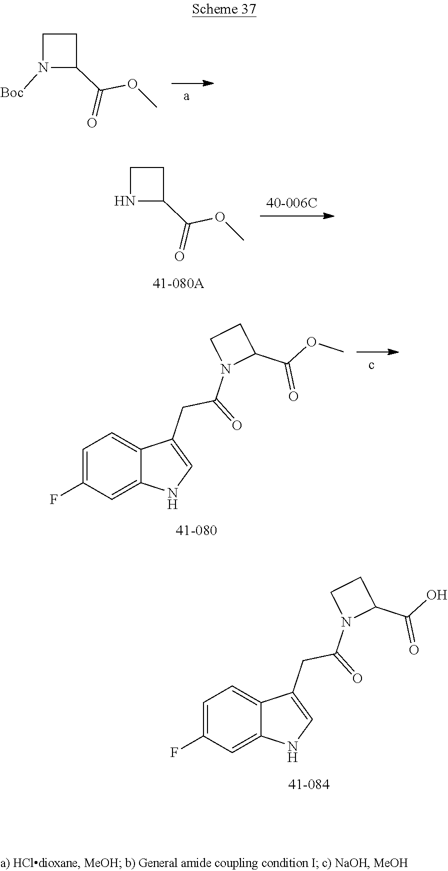

### methyl azetidine-2-carboxylate (41-080A)

The mixture of 1-(tert-butyl) 2-methyl azetidine-1,2-dicarboxylate (500 mg, 2.32 mmol) in HCl/dioxane (15 mL, 6 N) and MeOH (15 mL) was stirred at room temperature for 2 hours. Then, the mixture was concentrated to get crude product for the next step without further purification.

MS (ESI+) m/z 116 (M+H)⁺.

### Methyl 1-(2-(6-fluoro-1H-indol-3-yl)acetyl)azetidine-2-carboxylate (41-080) (Compound #10088)

Following the general amide coupling condition I, the desired product was obtained (180 mg, 59.9% yield) as white solid.

MS (ESI+) m/z 291 (M+H)⁺.

¹H NMR (400 MHz, CDCl₃) δ 8.21 (s, 1H), 7.56-7.47 (m, 1H), 7.16-7.11 (m, 0.6H), 7.05-6.98 (m, 1.4H), 6.93-6.85 (m, 1H), 4.81-4.73 (m, 0.6H), 4.70-4.65 (m, 0.4H), 4.26-4.17 (m, 0.6H), 4.12-3.94 (m, 1.4H), 3.78-3.71 (m, 3H), 3.66-3.55 (m, 2H), 2.62-2.47 (m, 1H), 2.29-2.16 (m, 1H).

### 1-(2-(6-fluoro-1H-indol-3-yl) acetyl) azetidine-2-carboxylic acid (41-084) (Compound #10102)

Methyl 1-(2-(6-fluoro-1H-indol-3-yl) acetyl) azetidine-2-carboxylate (90 mg, 0.31 mmol) was dissolved in MeOH/H₂O=10:1 (5.5 mL), then NaOH (1 mL, 1.0 M) was added dropwise to above solution. And the resulting mixture was stirred for 12 hours at room temperature. HCl (1 N) was added dropwise to the system till the pH=3, then the precipitate was filtered and washed with water (3 mL) to obtain 1-(2-(6-fluoro-1H-indol-3-yl)acetyl)azetidine-2-carboxylic acid (54 mg, 63.1% yield) as a white solid.

MS (ESI+) m/z 277 (M+H)⁺.291

¹H NMR (400 MHz, CDCl₃) δ 8.16 (s, 1H), 7.55-7.46 (m, 1H), 7.15-7.10 (m, 1H), 7.10-7.04 (m, 1H), 6.98-6.89 (m, 1H), 5.09-4.98 (m, 1H), 4.19-4.00 (m, 2H), 3.66 (s, 2H), 2.77-2.64 (m, 1H), 2.55-2.41 (m, 1H).

### Example 56—Synthesis of 2-(6-fluoro-1H-indol-3-yl)-1-(3-(2-methoxyethoxy) azetidin-1-yl) ethan-1-one (Compound #10103) (41-088 in Scheme 38)

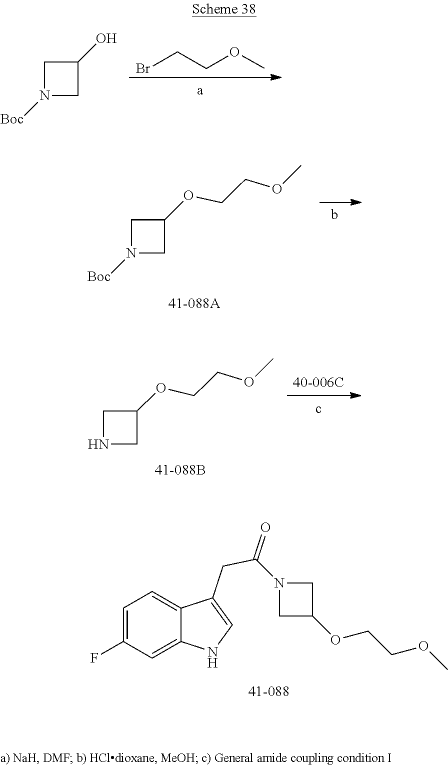

### tert-Butyl 3-(2-methoxyethoxy) azetidine-1-carboxylate(41-088A)

To a solution of tert-butyl 3-hydroxyazetidine-1-carboxylate (500 mg, 2.88 mmol) in DMF (20 mL) was added NaH (300 mg, 0.75 mmol) at 0° C. by portions. Then 1-bromo-2-methoxyethane (400 mg, 2.87 mmol) was added dropwise, and the mixture was stirred for 12 hours. After which period, the reaction was quenched with water (50 mL), and extracted with EtOAc (15 mL×3). The combined organic layers were dried over Na₂SO₄, concentrated to dryness. The residue was purified by flash column (elute with EtOAc:PE=0:1 to 1:1) to get tert-butyl 3-(2-methoxyethoxy) azetidine-1-carboxylate (330 mg, 68.6% yield) as a colorless oil.

MS (ESI+) m/z 232 (M+H)⁺.

### 3-(2-methoxyethoxy) azetidine(41-088B)

The mixture of tert-butyl 3-(2-methoxyethoxy) azetidine-1-carboxylate (330 mg, 1.42 mmol) in HCl/dioxane (15 mL, 6 N) and MeOH (15 mL) was stirred at room temperature for 2 hours. Then, the mixture was concentrated to get crude product for the next step without further purification.

MS (ESI+) m/z 132 (M+H)⁺.

### 2-(6-fluoro-1H-indol-3-yl)-1-(3-(2-methoxyethoxy) azetidin-1-yl) ethan-1-one (41-088) Compound #10103

Following the general amide coupling condition I, the desired product was obtained (80 mg, 50.4% yield) as a white solid.

MS (ESI+) m/z 307 (M+H)⁺.

¹H NMR (400 MHz, CDCl₃) δ 8.23 (s, 1H), 7.55-7.47 (m, 1H), 7.09-7.05 (m, 1H), 7.04-6.98 (m, 1H), 6.93-6.83 (m, 1H), 4.33-4.15 (m, 3H), 4.07-4.00 (m, 1H), 3.98-3.91 (m, 1H), 3.57 (s, 2H), 3.56-3.49 (m, 4H), 3.37 (s, 3H).

### Example 57—Synthesis of Further Compounds Using the Procedure of Example 56

Using the above procedures, the following examples were synthesized:

TABLE 19

| | Compound | | LC-MS |
|---|---|---|---|
| Structure | # | ¹H NMR | (M + H)⁺ |
| 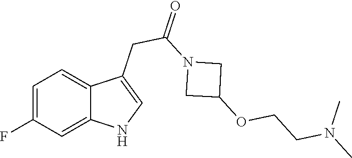 [SMILES: CN(C)CCOC1CN(C(=O)Cc2c[nH]c3cc(F)ccc23)C1] | 10110 | ¹H NMR (400 MHz, DMSO-d₆) δ 10.96 (s, 1H), 7.54-7.45 (m, 1H), 7.23-7.17 (m, 1H), 7.13- 7.07 (m, 1H), 6.87-6.79 (m, 1H), 4.36- 4.22 (m, 2H), 4.04-3.91 (m, 2H), 3.65-3.57 (m, 1H), 3.49 (s, 2H), 3.43-3.38 (m, 2H), 2.41- 2.32 (m, 2H), 2.12 (s, 6H). | 320 |

### Example 58—Synthesis of 2-(6-fluoro-1H-indol-3-yl)-1-(3-(1-hydroxyethyl)azetidin-1-yl)ethan-1-one (Compound #10131) (41-110 in Scheme 39)

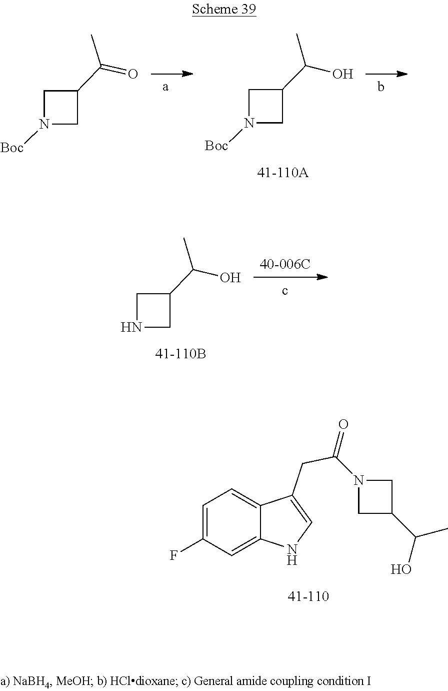

### tert-Butyl 3-(1-hydroxyethyl)azetidine-1-carboxylate (41-110A)

To a solution of tert-butyl 3-acetylazetidine-1-carboxylate1 (950 mg, 4.76 mmol) in MeOH (20 mL) was added NaBH₄ (360 mg, 9.53 mmol) slowly, and the resulting mixture was stirred at room temperature for 2 hours. The reaction was quenched by water (20 mL), extracted with EtOAc (10 mL×3). The combined organic layers were dried over sodium sulfate, filtered and concentrated to dryness. The residue was purified by column chromatography to obtain tert-butyl 3-(1-hydroxyethyl) azetidine-1-carboxylate (936 mg, 97.5% yield) as a colorless oil.

MS (ESI+) m/z 202 (M+H)⁺.

### 1-(azetidin-3-yl) ethan-1-ol (41-110B)

The mixture of tert-butyl 3-(1-hydroxyethyl) azetidine-1-carboxylate (200 mg, 0.99 mmol) in HCl/dioxane (5 mL, 6 N) and MeOH (5 mL) was stirred at room temperature for 2 hours. Then, the mixture was concentrated to get crude product for the next step without further purification.

MS (ESI+) m/z 102 (M+H)⁺.

### 2-(6-fluoro-1H-indol-3-yl)-1-(3-(1-hydroxyethyl)azetidin-1-yl)ethan-1-one (41-110) (Compound #10131)

Following the general amide coupling condition I, the desired product was obtained (28 mg, 19.6% yield) as white solid.

MS (ESI+) m/z 277 (M+H)⁺.

¹H NMR (400 MHz, DMSO-d₆) δ 10.94 (s, 1H), 7.54-7.45 (m, 1H), 7.19 (s, 1H), 7.10 (dd, J=10.2, 2.3 Hz, 1H), 6.88-6.78 (m, 1H), 4.75 (d, J=4.9 Hz, 1H), 4.13-4.04 (m, 1H), 4.02-3.97 (m, 0.5H), 3.92-3.86 (m, 0.5H), 3.82-3.73 (m, 1H), 3.72-3.62 (m, 1.5H), 3.61-3.52 (m, 0.5H), 3.49-3.41 (m, 2H), 2.47-2.38 (m, 1H), 1.01-0.93 (m, 3H).

### Example 59—Synthesis of 2-(6-fluoro-1H-indol-3-yl)-1-(3-(1-methoxyethyl)azetidin-1-yl)ethan-1-one (Compound #10124) (41-121 in Scheme 40)

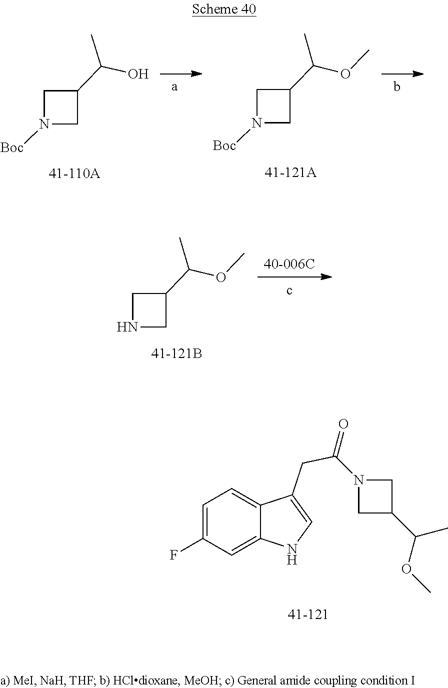

### tert-butyl 3-(1-methoxyethyl) azetidine-1-carboxylate (41-121A)

To a solution of tert-butyl 3-(1-hydroxyethyl) azetidine-1-carboxylate (680 mg, 3.37 mmol) in THE (25 mL) was added NaH (200 mg, 3.00 mmol) slowly, and the mixture was stirred at 0° C. for 0.5 hours. Then Mel (500 mg, 3.52 mmol) was added dropwise to above suspension, and the resulting mixture was stirred for 12 hours. After which period, the reaction was quenched by water (20 mL), extracted with EtOAc (10 mL×3). The combined organic layers were dried over sodium sulfate. After filtration and concentration to dryness, the residue was purified by column chromatography (silica gel, ethyl acetate:n-heptane=1:1) to obtain tert-butyl 3-(1-methoxyethyl) azetidine-1-carboxylate (672 mg, 92.4% yield) as a colorless oil.

MS (ESI+) m/z 216 (M+H)⁺.

### 3-(1-methoxyethyl) azetidine(41-121B)

The mixture of tert-butyl 3-(1-methoxyethyl) azetidine-1-carboxylate (300 mg, 1.39 mmol) in HCl/dioxane (5 mL, 6 N) and MeOH (5 mL) was stirred at room temperature for 2 hours. Then, the mixture was concentrated to get crude product for the next step without further purification.

MS (ESI+) m/z 116 (M+H)⁺.

### 2-(6-fluoro-1H-indol-3-yl)-1-(3-(1-methoxyethyl)azetidin-1-yl)ethan-1-one (41-121) (Compound #10124)

Following the general amide coupling condition I, the desired product was obtained (108 mg, 71.9% yield) as white solid.

MS (ESI+) m/z 291 (M+H)⁺.

¹H NMR (400 MHz, DMSO-d₆) δ 10.95 (s, 1H), 7.54-7.45 (m, 1H), 7.19 (s, 1H), 7.15-7.04 (m, 1H), 6.88-6.77 (m, 1H), 4.18-4.07 (m, 1H), 4.00-3.93 (m, 0.5H), 3.93-3.86 (m, 0.5H), 3.81 (t, J=9.1 Hz, 1H), 3.68-3.60 (m, 0.5H), 3.59-3.51 (m, 0.5H), 3.51-3.43 (m, 2H), 3.43-3.34 (m, 1H), 3.24 (s, 3H), 2.60-2.52 (m, 1H), 1.00 (d, J=6.1 Hz, 3H).

### Example 60—Synthesis of 2-(6-fluoro-1H-indol-2-yl)-1-(3-methylazetidin-1-yl)ethan-1-one (Compound #10143) (40-111 in Scheme 41)

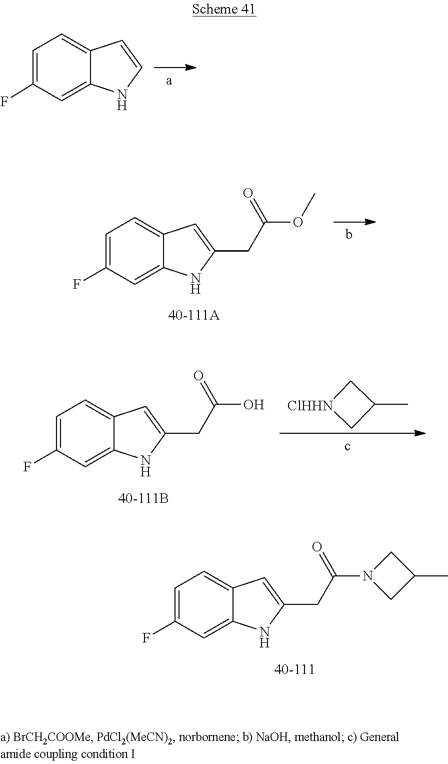

### Methyl 2-(6-fluoro-1H-indol-2-yl) acetate (40-11 IA)

To a vial were added 6-fluoro-1H-indole (500 mg, 3.70 mmol), methyl 2-bromoacetate (679.2 mg, 4.44 mmol), Pd(CH₃CN)₂Cl₂ (192.0 mg, 0.74 mmol), norbornene (696.7 mg, 7.40 mmol), NaHCO₃ (1.2 g, 14.80 mmol), water (66.6 mg, 3.70 mmol) and DMF (15 mL). The reaction mixture was reacted in microwave reactor under N₂ atmosphere at 70° C. for 2 hours. After cooling to room temperature, the mixture was diluted with water (150 mL), extracted with ethyl acetate (40 mL). The separated organic phase was dried over sodium sulfate, filtered and concentrated to dryness. The residue was purified by flash column to obtain title compound as yellow solid (420 mg, 54.8% yield).

MS (ESI+) m/z 208 (M+H)⁺.

¹H NMR (400 MHz, DMSO-d₆) δ 11.12 (s, 1H), 7.43 (dd, J=8.6, 5.5 Hz, 1H), 7.10 (dd, J=10.1, 2.3 Hz, 1H), 6.83-6.78 (m, 1H), 6.29-6.27 (m, 1H), 3.83 (s, 2H), 3.65 (s, 3H).

### 2-(6-fluoro-1H-indol-2-yl) acetic acid (40-111B)

To the solution of methyl 2-(6-fluoro-1H-indol-2-yl) acetate (400 mg, 1.93 mmol) in methanol (10 mL) was added sodium hydroxide (10 mL, 1.0 N). Then, the mixture was stirred at room temperature for 16 hours. The solvent was removed by reduced pressure and the residual aqueous solution was neutralized with conc. hydrochloride acid. The precipitate was filtered and washed with water (5 mL*2) to get 2-(6-fluoro-1H-indol-2-yl) acetic acid (320 mg, 85.8% yield) as yellow solid.

MS (ESI+) m/z 194 (M+H)⁺.

### 2-(6-fluoro-1H-indol-2-yl)-1-(3-methylazetidin-1-yl)ethan-1-one (40-111) (Compound #10143)

Following the general amide coupling condition I, the desired product was obtained as white solid (70 mg, 54.9% yield).

MS (ESI+) m/z 247 (M+H)⁺.

¹H NMR (400 MHz, CDCl₃) δ 9.25 (s, 1H), 7.43-7.40 (m, 1H), 7.03-7.00 (m, 1H), 6.85-6.80 (m, 1H), 6.24-6.21 (m, 1H), 4.33 (t, J=8.3 Hz, 1H), 4.14 (t, J=9.1 Hz, 1H), 3.79-3.76 (m, 1H), 3.65-3.52 (m, 3H), 2.82-2.61 (m, 1H), 1.25 (d, J=6.9 Hz, 3H).

### Example 61—Synthesis of 2-(6-fluoro-1H-indol-5-yl)-1-(3-methylazetidin-1-yl) ethan-1-one (Compound #10176) (40-146 in Scheme 42)

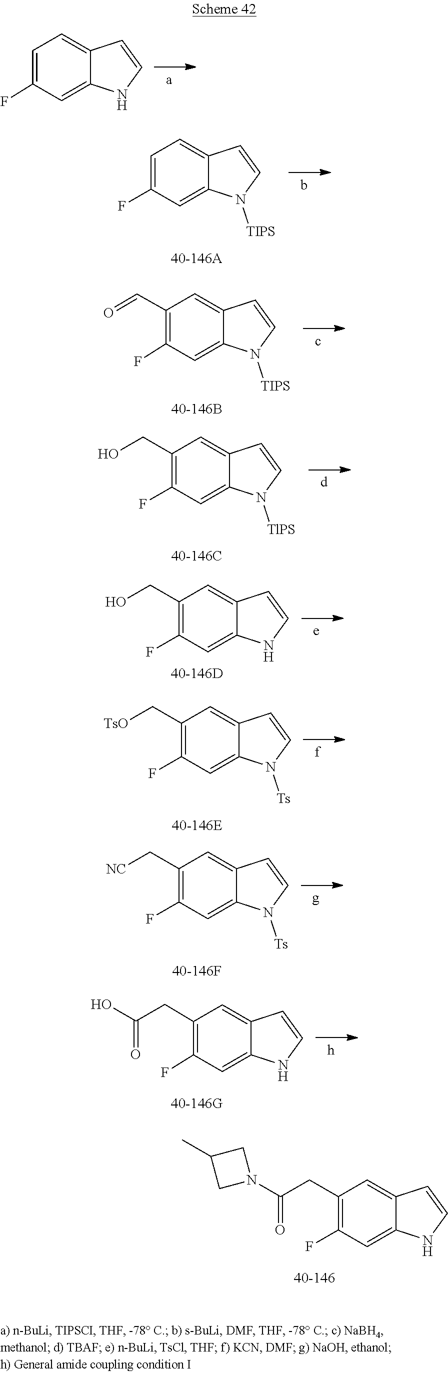

### 6-fluoro-1-(triisopropylsilyl)-1H-indole (40-146A)

To a solution of 6-fluoro-1H-indole (3.2 g, 23.68 mmol) dissolved in anhydrous tetrahydrofuran (50 mL) was added n-BuLi (9.5 mL, 23.68 mmol) dropwise at −78° C. under argon. The reaction mixture was stirred at −78° C. for 20 minutes, then triisopropylsilane chloride (5.1 mL, 23.68 mmol) was added dropwise. The mixture was allowed to warm to room temperature spontaneously and stirred for 40 minutes. After which period, water was added to quench the reaction. The system was extracted with ethyl acetate, and the separated organic phase was dried over sodium sulfate. After filtration and concentration to dryness, the residue was purify by flash column (PE:EA=99:1) to obtain title compound as colorless oil (6.9 g, 99% yield).

¹H NMR (400 MHz, CDCl₃) δ 7.51 (dd, J=8.6, 5.8 Hz, 1H), 7.30-7.12 (m, 2H), 6.98-6.81 (m, 1H), 6.60-6.57 (m, 1H), 1.71-1.64 (m, 3H), 1.22-1.08 (m, 18H).

### 6-fluoro-1-(triisopropylsilyl)-1H-indole-5-carbaldehyde (40-146B)

A solution of 6-fluoro-1-(triisopropylsilyl)-1H-indole (6.0 g, 20.58 mmol) in tetrahydrofuran (50 mL) was cooled to −78° C., and a 1.3 M solution of sec-butyllithium in hexanes (15.8 mL, 20.58 mmol) was added dropwise. The resulting mixture was stirred for 2 hours at −78° C. before 7.8 mL dimethylformamide was added slowly. The reaction was allowed to warm up to room temperature and stirred for 2 more hours. Water was added and the aqueous phase was extracted with ethyl acetate. The organic layer was washed with brine and dried over sodium sulfate. After filtration and concentration to dryness, the residue was purify by flash column (PE:EA=50:1) to obtain title compound as yellow oil (4.2 g, 63.8% yield).

MS (ESI+) m/z 164 (M+H)⁺.

### (6-fluoro-1-(triisopropylsilyl)-1H-indol-5-yl) methanol (40-146C)

6-fluoro-1-(triisopropylsilyl)-1H-indole-5-carbaldehyde (1.5 g, 4.70 mmol) was dissolved in methanol (15 mL) and sodium borohydride (355.2 mg, 9.39 mmol) was added by portions thereto at 0° C. The mixture was stirred at 0° C. for 2 hours and then water was added thereto. The solution was extracted with ethyl acetate, and the separated organic phase was washed with brine, dried over sodium sulfate. After filtration, the filtrate was concentrated to get crude product as yellow solid (1.6 g) for the next step without further purification.

### (6-fluoro-1H-indol-5-yl)methanol (40-146D)

The solution of (6-fluoro-1-(triisopropylsilyl)-1H-indol-5-yl) methanol (1.4 g, 4.35 mmol) in TBAF (12 mL, 1.0 M in THF) was stirred at room temperature for 1 hour. The mixture was diluted with ethyl acetate (60 mL), washed with water (40 mL), dried over sodium sulfate. After filtration and concentration to dryness, the residue was purified by flash column (PE:EA=2:1) to obtain desired product as white solid (660 mg, 80.3% yield).

¹H NMR (400 MHz, DMSO-d₆) δ 11.05 (s, 1H), 7.54 (d, J=7.4 Hz, 1H), 7.30-7.28 (m, 1H), 7.11 (d, J=11.1 Hz, 1H), 6.42-6.40 (m, 1H), 5.04 (t, J=5.7 Hz, 1H), 4.58-4.56 (m, 2H).

### (6-fluoro-1-tosyl-1H-indol-5-yl) methyl 4-methylbenzenesulfonate (40-146E)

To a solution of (6-fluoro-1H-indol-5-yl) methanol (400 mg, 2.42 mmol) in dry tetrahydrofuran (20 mL) was added n-BuLi (2.0 ml, 5.09 mmol) dropwise at −78° C. Then, the mixture was stirred at −78° C. for 45 min, and 4-methylbenzenesulfonyl chloride (969.6 mg, 5.09 mmol) in tetrahydrofuran (3 mL) was added dropwise. After addition, the mixture was stirred at −78° C. for 20 minutes and continued to stir for 30 minutes at room temperature. The reaction was quenched with water (30 mL), extracted with ethyl acetate (30 mL). The separated organic phase was washed with brine (30 mL), dried over sodium sulfate, filtered and concentrated to get crude product (1.2 g) for the next step without further purification.

### 2-(6-fluoro-1-tosyl-1H-indol-5-yl) acetonitrile (40-146F)

The solution of (6-fluoro-1-tosyl-1H-indol-5-yl) methyl 4-methylbenzenesulfonate (1.1 g, 2.42 mmol) and cyan potassium (315.4 mg, 4.84 mmol) in DMF (10 mL) was stirred at 60° C. for 2 hours. After cooling to room temperature, the mixture was diluted with water (80 mL), which was then extracted with ethyl acetate (40 mL). The separated organic phase was dried over sodium sulfate, filtered and concentrated to dryness. The residue was purified by flash column to obtain colorless gel (350 mg, 44.0% yield).

¹H NMR (400 MHz, DMSO-d₆) δ 7.93-7.91 (m, 2H), 7.86-7.84 (m, 1H), 7.79 (d, J=10.6 Hz, 1H), 7.69 (d, J=7.4 Hz, 1H), 7.40 (d, J=8.2 Hz, 2H), 6.89 (d, J=3.6 Hz, 1H), 4.09 (s, 2H), 2.33 (s, 3H).

### 2-(6-fluoro-1H-indol-5-yl) acetic acid (40-146G)

The solution of 2-(6-fluoro-1-tosyl-1H-indol-5-yl) acetonitrile (320 mg, 0.97 mmol) in ethanol (6 mL) was added aqueous sodium hydroxide (6 mL, 20% W/V). Then, the reaction was stirred at 100° C. for 3 hours. After cooling to room temperature, the mixture was concentrated in vacuo and the residue was added conc. HCl at 0° C. till pH-2. The precipitate was filtered and washed with water (20 mL). The yellow solid (150 mg, 63.7% yield) collected was dried and used for the next step without further purification.

MS (ESI+) m/z 194 (M+H)⁺.

### 2-(6-fluoro-1H-indol-5-yl)-1-(3-methylazetidin-1-yl) ethan-1-one (40-146) (Compound #10176)

Following the general amide coupling condition I, the desired product was obtained as white solid (74 mg, 44.6% yield).

MS (ESI+) m/z 247 (M+H)⁺.

¹H NMR (400 MHz, DMSO-d₆) δ 11.05 (s, 1H), 7.38 (d, J=7.5 Hz, 1H), 7.30-7.28 (m, 1H), 7.11 (d, J=10.8 Hz, 1H), 6.43-6.31 (m, 1H), 4.26 (t, J=8.3 Hz, 1H), 3.95 (t, J=8.8 Hz, 1H), 3.73-3.69 (m, 1H), 3.51-3.38 (m, 3H), 2.73-2.59 (m, 1H), 1.18 (d, J=6.9 Hz, 3H).

### Example 62—Synthesis of 1-(azetidin-1-yl)-2-(5-fluoro-1H-pyrrolo[2,3-b]pyridin-3-yl)ethan-1-one (Compound #10035) (40-029 in Scheme 43)

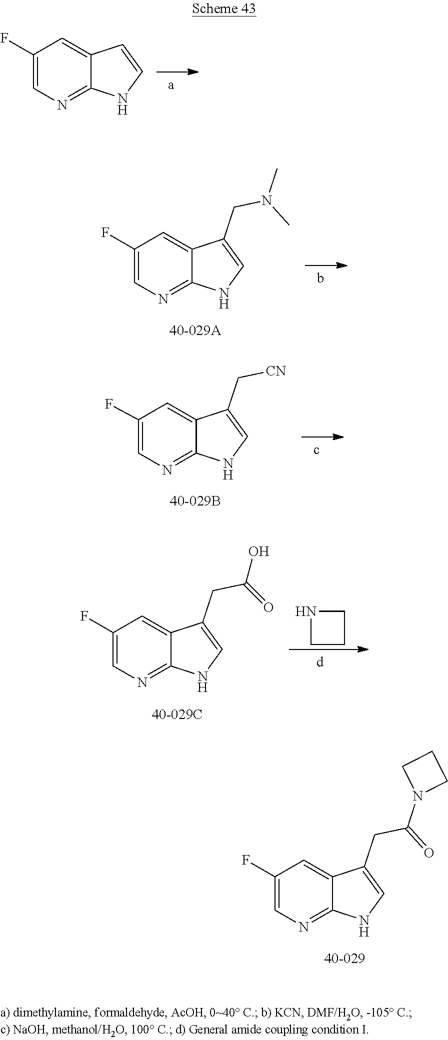

### 1-(5-fluoro-1H-pyrrolo[2,3-b]pyridin-3-yl)-N, N-dimethylmethanamine (40-029A)

The solution of 40 percent aq. dimethylamine (1.0 g, 8.81 mmol) was cooled to 5° C., and glacial acetic acid (1.1 mL) was added dropwise while maintaining the temperature at ˜15° C. After stirring for 20 minutes at about 3° C., 37 percent aqueous formaldehyde (0.6 mL, 8.81 mmol) was slowly added while keeping the temperature between 0˜10° C. 5-fluoro-1H-pyrrolo[2,3-b]pyridine (1 g, 7.35 mmol) was added. The reaction was exothermic and reached a final temperature˜40° C., and it was then cooled down to ˜20° C. The reaction solution was slowly added to 16 mL aqueous 3M sodium hydroxide. The suspension was stirred about 30 min and then filtered, rinsed with water (5 mL*2) to afford the desired product as yellow solid (840 mg, 59.2% yield).

MS (ESI+) m/z 194 (M+H)⁺.

### 2-(5-fluoro-1H-pyrrolo[2,3-b]pyridin-3-yl)acetonitrile (40-029B)

The mixture of 1-(5-fluoro-1H-pyrrolo[2,3-b]pyridin-3-yl)-N,N-dimethylmethanamine (420 mg, 2.17 mmol), KCN (198.2 g, 3.04 mmol), DMF (4 mL) and water (2 mL) were heated to 105° C. for 10 hours. The reaction mixture was cooled to 25° C., water (20 mL) and toluene (15 mL) were added thereto and stirred for 3 hours. The organic and aqueous layers were separated. The organic layer was washed with aqueous NaHCO₃ (10 mL) and brine (10 mL), dried over sodium sulfate, filtered and concentrated to get crude product as yellow oil for the next step without further purification (400 mg).

MS (ESI+) m/z 176 (M+H)⁺.

### 2-(5-fluoro-1H-pyrrolo[2,3-b]pyridin-3-yl)acetic acid (40-029C)

The mixture of 2-(5-fluoro-1H-pyrrolo[2,3-b]pyridin-3-yl)acetonitrile (400 mg, 2.28 mmol), sodium hydroxide (730.8 mg, 18.27 mmol), methanol (3 mL) and water (9 mL) was stirred at 100° C. for 3 hours. Then, the reaction was cooled to 0° C. and treated with 6 N aqueous solution of HCl to pH-1. The mixture was extracted with ethyl acetate (50 mL), and the organic phase was dried over sodium sulfate, filtered and concentrated to obtain desired product as yellow oil (320 mg, 72.2% yield).

MS (ESI+) m/z 195 (M+H)⁺.

### 1-(azetidin-1-yl)-2-(5-fluoro-1H-pyrrolo[2,3-b]pyridin-3-yl)ethan-1-one (40-029) (Compound #10035)

Following the general amide coupling condition I, the desired product was obtained as white solid (53 mg, 36.8% yield).

MS (ESI+) m/z 234 (M+H)⁺.

¹H NMR (400 MHz, CDCl₃) δ 9.36 (s, 1H), 8.22-8.14 (m, 1H), 7.80 (dd, J=8.8, 2.6 Hz, 1H), 7.32 (s, 1H), 4.19 (t, J=7.6 Hz, 2H), 4.06 (t, J=7.8 Hz, 2H), 3.52 (s, 2H), 2.34-2.22 (m, 2H).

### Example 63—Synthesis of 1-(azetidin-1-yl)-2-(6-fluoro-2-methyl-1H-indol-3-yl)ethan-1-one (Compound #10077) (40-068 in Scheme 44)

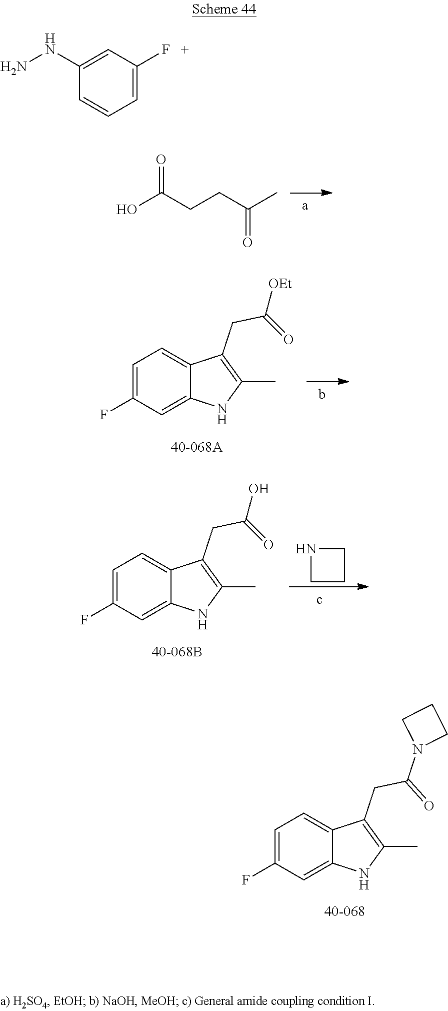

### Ethyl 2-(6-fluoro-2-methyl-1H-indol-3-yl)acetate (40-068A)

A mixture of (3-fluorophenyl)hydrazine hydrochloride salt (7.0 g, 43.05 mmol), ethyl 4-oxopentanoate (4.6 g, 39.62 mmol) and ethanol (48 mL) was treated with sulfuric acid (2.0 mL), and the resulting reaction mixture was stirred at 85° C. for 3 days. The mixture was cooled to room temperature, poured onto a mixture of ice and water (200 mL) and extracted with dichloromethane (100 mL, 3 times). The combined extracts were washed with saturated aqueous sodium chloride solution (20 mL) and dried over magnesium sulfate. The solvent was removed under reduced pressure, and the residue was purified by column chromatography to get ethyl 2-(6-fluoro-2-methyl-1H-indol-3-yl)acetate as yellow oil (320 mg, 2.5% yield).

MS (ESI+) m/z 236 (M+H)⁺.

¹H NMR (400 MHz, CDCl₃) δ 8.01 (s, 1H), 7.04-6.91 (m, 2H), 6.72-6.67 (m, 1H), 4.17 (q, J=7.1 Hz, 2H), 3.79 (s, 2H), 2.30 (s, 3H), 1.26 (t, J=7.1 Hz, 3H).

### 2-(6-fluoro-2-methyl-1H-indol-3-yl) acetic Acid (40-068B)

The solution of ethyl 2-(6-fluoro-2-methyl-1H-indol-3-yl) acetate (200 mg, 0.85 mmol) in methanol (3 mL) was added sodium hydroxide (3 mL, 1.0 N). Then, the mixture was stirred at room temperature for 16 hours. The solvent was evaporated, HCl (4 mL, 1.0 N) was added to adjust pH<7. The precipitate formed was filtered and washed with water (3 mL) to obtain title compound as yellow solid (100 mg, 56.8% yield).

MS (ESI+) m/z 222 (M+H)⁺.

### 1-(azetidin-1-yl)-2-(6-fluoro-2-methyl-1H-indol-3-yl)ethan-1-one (40-068) (Compound #10077)

Following the general amide coupling condition I, the desired product was obtained as white solid (80 mg, 67.3% yield).

MS (ESI+) m/z 247 (M+H)⁺.

¹H NMR (400 MHz, DMSO-d₆) δ 11.07 (s, 1H), 7.07-7.05 (m, 1H), 6.94-6.88 (m, 1H), 6.65-6.6. (m, 1H), 4.14 (t, J=7.7 Hz, 2H), 3.83 (t, J=7.7 Hz, 2H), 3.47 (s, 2H), 2.28 (s, 3H), 2.25-2.11 (m, 2H).

### Example 64—Synthesis of 2-(6-fluoro-1H-indazol-3-yl)-1-(3-methylazetidin-1-yl)ethan-1-one (Compound #10144) (41-145 in Scheme 45)

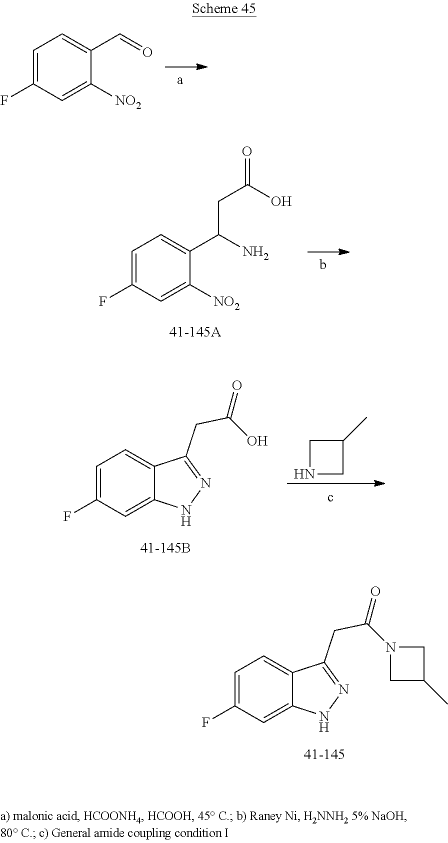

### 3-amino-3-(4-fluoro-2-nitrophenyl) propanoic acid (41-145A)

4-Fluoro-2-nitrobenzaldehyde (2.5 g, 14.78 mmol), formic acid (2.25 mL, 0.05 mol) and malonic acid (2.00 g, 19.21 mmol) were stirred at 45° C. for half an hour, and then ammonium formate (2.33 g, 36.94 mmol) was added thereto. The reaction temperature was raised to 70° C. and stirred for 1 hour, and then stirred at 95° C. for another 4 hours. Then concentrated hydrochloric acid (5.6 mL) was added and stirred maintaining this temperature for another 1 hour. After which period, the system was cooled, water (20 mL) was added, and extracted with ethyl acetate (25 mL*2). The organic phase was discarded, the aqueous phase was adjusted to pH≈4 with 50 percent potassium hydroxide solution. A solid was precipitated, pumping filtered and dried in vacuum to obtain title compound as yellow solid (1.5 g, 44.5% yield).

### 2-(6-fluoro-1H-indazol-3-yl) acetic acid (41-145B)

3-Amino-3-(4-fluoro-2-nitrophenyl) propionic acid (1.5 g, 6.57 mmol) was dissolved in a mixed solution of 5 percent sodium hydroxide solution (8 mL) and 85 percent hydrazine hydrate (0.5 mL). The reaction was heated to 80° C., and then Raney nickel (2.5 mg*2) was added carefully and stirred for half an hour. Then it was cooled and adjusted to pH≈2 with 6 N hydrochloric acid. A solid precipitate was pumping filtered and dried in vacuum to obtain a yellow solid (340 mg, crude).

MS (ESI+) m/z 195 (M+H)⁺.

### 2-(6-fluoro-1H-indazol-3-yl)-1-(3-methylazetidin-1-yl)ethan-1-one (41-145) Compound #10144

Following the general amide coupling condition I, the desired product was obtained as white solid (25 mg, 11.5% yield) as white solid.

MS (ESI+) m/z 248 (M+H)⁺.

¹H NMR (400 MHz, CDCl₃) δ 10.13 (br s, 1H), 7.83-7.74 (m, 1H), 7.05-6.97 (m, 1H), 6.96-6.87 (m, 1H), 4.30 (t, J=8.4 Hz, 1H), 4.14 (t, J=9.1 Hz, 1H), 3.81 (s, 2H), 3.79-3.71 (m, 1H), 3.64-3.55 (m, 1H), 2.75-2.60 (m, 1H), 1.32-1.17 (m, 3H).

### Example 65—Synthesis of 2-(6-fluoro-1H-indol-4-yl)-1-(3-methylazetidin-1-yl) ethan-1-one (Compound #10172) (69-016 in Scheme 46)

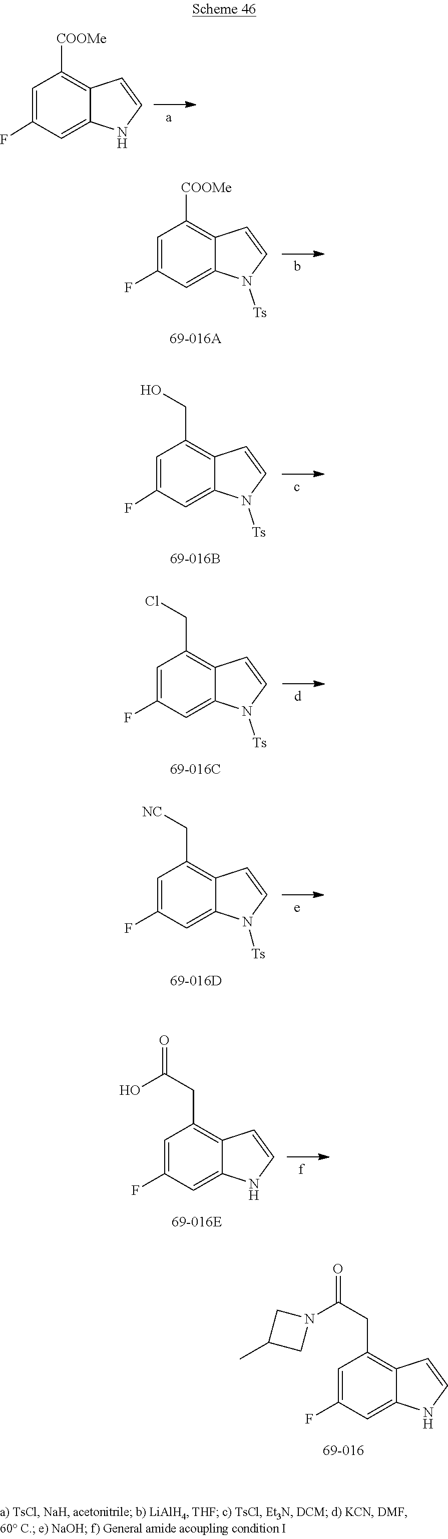

### Methyl 6-fluoro-1-tosyl-1H-indole-4-carboxylate (69-016A)

The solution of methyl 6-fluoro-1H-indole-4-carboxylate (900 mg, 4.66 mmol) in acetonitrile (25 mL) was cooled to 0° C. NaH (260.9 mg, 6.52 mmol) was added and stirred for 30 minutes. Then, p-toluenesulfonyl chloride (977.0 mg, 5.12 mmol) was added portion-wise, and the resulting mixture was stirred at room temperature for 2 hours. After which period, the reaction was quenched with saturated NH₄Cl solution (40 mL), extracted with ethyl acetate (100 mL). The separated organic phase was dried over sodium sulfate, filtered and concentrated to dryness, which was purified by flash column (PE:EA=8:1) to obtain yellow solid (1.6 g, 99% yield).

¹H NMR (400 MHz, DMSO-d₆) δ 8.05-8.03 (m, 2H), 7.98-7.95 (m, 2H), 7.69 (dd, J=9.8, 2.4 Hz, 1H), 7.43-7.41 (m, 2H), 7.26 (dd, J=3.7, 0.6 Hz, 1H), 3.90 (s, 3H), 2.33 (s, 3H).

### (6-fluoro-1-tosyl-1H-indol-4-yl) methanol (69-016B)

To the solution of methyl 6-fluoro-1-(4-methylphenyl) sulfonyl-indole-4-carboxylate (1.4 g, 4.03 mmol) in dry THE (45 mL) was added LiAIH₄ (8.1 mL, 8.06 mmol) dropwise at 0° C. Then, the mixture was stirred at room temperature for 2 hours. After cooling to 0° C. again, the mixture was treated with water (0.3 mL), 15 percent NaOH (0.3 mL) and water (0.9 mL), and filtered. The filtrate was concentrated in vacuo and the residue was purified by flash column to afford tittle compound as white solid (720 mg, 55.9% yield).

¹H NMR (400 MHz, DMSO-d₆) δ 7.91-7.89 (m, 2H), 7.79 (d, J=3.7 Hz, 1H), 7.58 (dd, J=9.6, 2.1 Hz, 1H), 7.40 (d, J=8.4 Hz, 2H), 7.11 (dd, J=10.4, 2.2 Hz, 1H), 6.91 (d, J=3.7 Hz, 1H), 5.38 (t, J=5.7 Hz, 1H), 4.69 (d, J=5.7 Hz, 2H), 2.32 (s, 3H).

### 4-(chloromethyl)-6-fluoro-1-tosyl-1H-indole (69-016C)

To the solution of [6-fluoro-1-(4-methylphenyl) sulfonyl-indol-4-yl] methanol (700 mg, 2.19 mmol) and Et₃N (0.6 mL, 4.39 mmol) in DCM (15 mL) was added 4-

methylbenzene sulfonyl chloride (626.8 mg, 3.29 mmol) by portions. Then, the mixture was stirred at room temperature for 16 hours. After which period, the reaction was diluted with DCM (30 mL), washed with water (30 mL), dried over sodium sulfate. After filtration and concentration to dryness, the residue was purified by flash column to obtain tittle compound as white solid (400 mg, 54.0% yield).

¹H NMR (400 MHz, DMSO-d₆) δ7.95-7.90 (m, 3H), 7.72 (dd, J=9.5, 1.8 Hz, 1H), 7.41 (d, J=8.2 Hz, 2H), 7.29 (dd, J=10.0, 2.1 Hz, 1H), 7.02 (d, J=3.6 Hz, 1H), 4.99 (s, 2H), 2.33 (s, 3H).

### 2-(6-fluoro-1-tosyl-1H-indol-4-yl) acetonitrile (69-016D)

The solution of 4-(chloromethyl)-6-fluoro-1-(4-methylphenyl) sulfonyl-indole (370 mg, 1.10 mmol) and potassium cyanide (85.6 mg, 1.31 mmol) in DMF (10 mL) was stirred at 6° C. for 3 hours. After cooling to room temperature, the mixture was diluted with water (100 mL), extracted with ethyl acetate (50 mL). The separated organic phase was dried over sodium sulfate, filtered and concentrated to dryness, which was then purify by Prep-HPLC to obtain title compound (250 mg, 69.5% yield).

¹H NMR (400 MHz, CDCl₃) δ 7.78-7.75 (m, 2H), 7.71 (dd, J=9.3, 1.8 Hz, 1H), 7.62 (d, J=3.8 Hz, 1H), 7.28-7.26 (m, 2H), 7.05 (dd, J=9.4, 2.1 Hz, 1H), 6.67 (dd, J=3.8, 0.7 Hz, 1H), 3.87 (s, 2H), 2.37 (s, 3H).

### 2-(6-fluoro-1H-indol-4-yl) acetic acid (69-016E)

The solution of 2-(6-fluoro-1-tosyl-1H-indol-4-yl) acetonitrile (220 mg, 0.67 mmol) in ethanol (5 mL) was added aqueous sodium hydroxide (5 mL, 20% WN). Then, the reaction mixture was stirred at 100° C. for 3 hours. After cooling to room temperature, the mixture was evaporated, and the residue was added conc. HCl at 0° C. till pH˜2. The precipitate was filtered and washed with water (20 mL). The yellow solid (100 mg, 77.3% yield) was dried and used for the next step without further purification.

MS (ESI+) m/z 194 (M+H)⁺.

### 2-(6-fluoro-1H-indol-4-yl)-1-(3-methylazetidin-1-yl) ethan-1-one (69-016) (Compound #10172)

Following the general amide coupling condition I, the desired product was obtained as white solid (35 mg, 27.5% yield).

MS (ESI+) m/z 247 (M+H)⁺.

¹H NMR (400 MHz, CDCl₃) δ 8.37 (s, 1H), 7.19-7.13 (m, 1H), 6.97 (dd, J=9.3, 1.9 Hz, 1H), 6.82 (dd, J=10.3, 2.1 Hz, 1H), 6.59 (s, 1H), 4.13 (t, J=8.8 Hz, 2H), 3.73 (s, 2H), 3.61-3.58 (m, 2H), 2.66-2.61 (m, 2H), 1.20 (d, J=6.9 Hz, 3H).

### Example 66—Synthesis of 2-(6-fluoro-1H-pyrrolo[3,2-b]pyridin-3-yl)-1-(3-methylazetidin-1-yl) ethan-1-one (Compound #10174) (70-026 in Scheme 47)

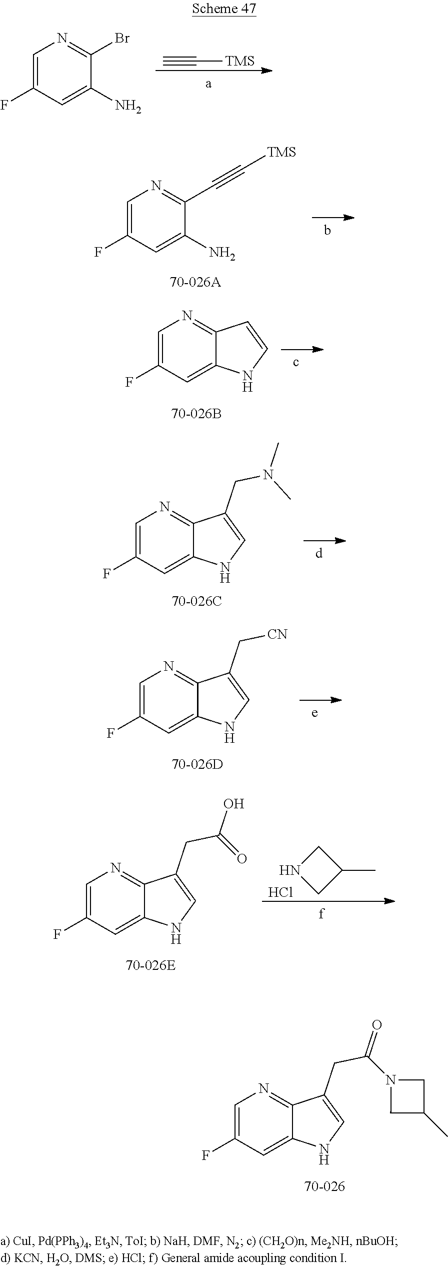

### 5-fluoro-2-((trimethylsilyl)ethynyl) pyridin-3-amine (70-026A)

A solution of 2-bromo-5-fluoropyridin-3-amine (5 g, 26.18 mmol), TEA (7.9 g, 78.53 mmol), Pd(PPh₃)₄ (1.8 g, 2.6 mmol) and Cul (490 mg, 2.6 mmol) in toluene (30 mL) at 0° C. under nitrogen was added a solution of (trimethylsilyl)acetylene (5.13 mL, 52.36 mmol). The mixture was stirred at 48° C. for 16 hours. After cooling to room temperature, the mixture was concentrated under reduce pressure and the residue was dissolved in hexane:MTBE (1:1, 100 mL). Then, the solution was filtered through a pad of silica (8 g), washed with hexane:MTBE (1:1, 500 mL). The filtrate was concentrated to obtain desired product as gray solid (2.5 g, 46% yield).

MS (ESI+) m/z 209 (M+H)⁺

### 6-fluoro-1H-pyrrolo[3,2-b]pyridine (70-026B)

NaH (1.7 g, 41.39 mmol) was added by portions to the solution of 5-fluoro-2-((trimethylsilyl)ethynyl) pyridin-3-amine (2 g, 10.35 mmol) in DMF (10 mL) at 0° C. After stirring at room temperature for 2 hours, the reaction mixture was poured into ice-cold water (30 mL) and extracted with EtOAc (30 mL×3). The separated organic phase was dried over sodium sulfate, filtered and concentrated to dryness, which was purified by flash column to obtain desired product as yellow solid (1.05 g, 75% yield).

MS (ESI+) m/z 137 (M+H)⁺.

### 1-(6-fluoro-1H-pyrrolo[3,2-b]pyridin-3-yl)-N,N-dimethylmethanamine (70-026C)

The solution of 3-fluoro-5,9-diazabicyclo[4.3.0]nona-1,3,5,7-tetraene (1 g, 7.35 mmol), formaldehyde (243 mg, 8.08 mmol) and dimethylamine hydrochloride (659 mg, 8.08 mmol) in n-butanol (20 mL) was heated to 118° C. for 3 hours. After cooling to room temperature, the resulting mixture was concentrated to dryness, which was purified by flash column to give desired product (834 mg, 59% yield).

MS (ESI+) m/z 194 (M+H)⁺.

### 2-(6-fluoro-1H-pyrrolo[3,2-b]pyridin-3-yl) acetonitrile (70-026D)

To the solution of 1-(3-fluoro-5,9-diazabicyclo [4.3.0]nona-1,3,5,7-tetraen-7-yl)-N,N-dimethyl-methenamine (834 mg, 4.32 mmol) in THE (20 mL) was added dimethyl sulfate (544.0 mg, 4.32 mmol). The resulting mixture was heated at reflux for 60 minutes. The solvent was removed under reduced pressure, and the residue was re-dissolved in water (20 mL). Then, KCN (309.0 mg, 4.75 mmol) was added and the mixture was refluxed for another 16 hours. After which period, the reaction was cooled down to room temperature and extracted with ethyl acetate (40 mL*3). The combined organic layers were dried over sodium sulfate, filtered and concentrated to dryness. The residue was purified by flash column to get desired product as yellow solid (420 mg, 54% yield).

MS (ESI+) m/z 176 (M+H)⁺.

### 2-(6-fluoro-1H-pyrrolo[3,2-b]pyridin-3-yl) acetic acid (70-026E)

The solution of 2-(3-fluoro-5,9-diazabicyclo [4.3.0] nona-1,3,5,7-tetraen-7-yl) acetonitrile (150 mg, 0.86 mmol) in conc. HCl (4 mL) was stirred at 100° C. for 1 hour. After cooling to room temperature, the mixture was concentrated to obtain desired product for the next step without further purification.

MS (ESI+) m/z 195 (M+H)⁺.

### 2-(6-fluoro-1H-pyrrolo[3,2-b]pyridin-3-yl)-1-(3-methylazetidin-1-yl) ethan-1-one (70-026) (Compound #10174)

Following the general amide coupling condition I, the desired product was obtained as white solid (30 mg, 16% yield).

MS (ESI+) m/z 248 (M+H)⁺.

¹H NMR (400 MHz, DMSO-d₆) δ 11.21 (s, 1H), 8.36-8.27 (m, 1H), 7.66-7.59 (m, 1H), 7.51 (s, 1H), 4.37-4.29 (m, 1H), 3.99-3.90 (m, 1H), 3.84-3.75 (m, 1H), 3.49 (s, 2H), 3.43-3.35 (m, 1H), 2.70-2.56 (m, 1H), 1.18 (d, J=6.9 Hz, 3H).

¹H NMR (400 MHz, DMSO-d₆) δ 10.97 (s, 1H), 10.65 (s, 1H), 8.63 (d, J=4.8 Hz, 2H), 7.58-7.55 (m, 1H), 7.25 (d, J=2.3 Hz, 1H), 7.19-7.08 (m, 2H), 6.87-6.82 (m, 1H), 3.87 (s, 2H).

### Example 67—Synthesis of Compound #11006, 11007, 11039 and 11040

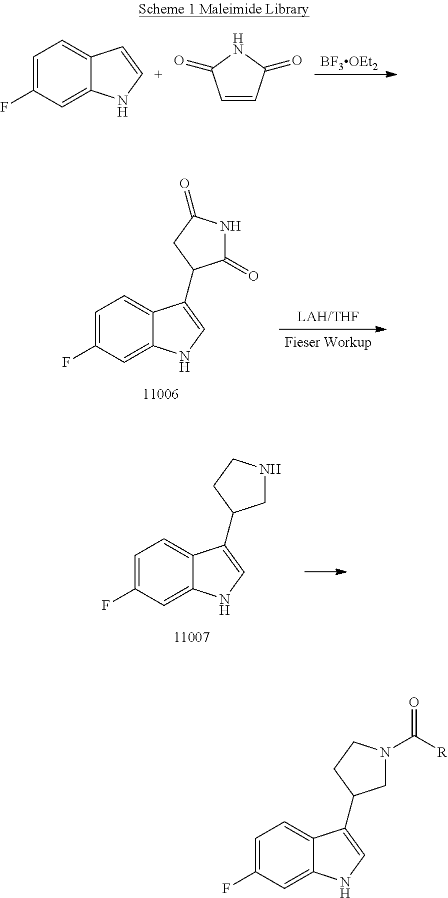

Compound #11006: Michael Addition for C—C Bond Formation on Position 3 of Indole Catalyzed by BF₃·OEt₂

To an oven dried 2 neck round-bottom flask was added 6-fluoroindole (200 mg, 1.48 mmol) followed by 143.58 mg (1.48 mmol) of maleimide. DCE was added to the reaction mixture while mixing under the nitrogen gas. Catalyst BF₃·OEt₂ (63, 017 mg, 0.44 mmol) was added to the mixture. The reaction was heated to reflux at 85° C. for 18 hrs. The reaction was cooled down after 18 hrs. The DCE was evaporated. The reaction mixture was worked up using water (15 mL) and EtOAc (2×15). The organic layer was dried using anhydrous sodium sulphate and filtered. After evaporation of the solvent the crude product was purified by Biotage using EtOAC/Heptane to provide pure Compound #11006 (50%) as racemic mixture.

### Compound #11007: Hydrogenation Reaction

Compound #11006 (50 mg, 0.21 mmol) was dissolved whilst stirring in a suspension of LiAlH (1.077 mmol, 5 eq.) in anhydrous THE (3 mL, 0° C., under N2 gas). The solution was stirred at 65° C. for 16 h (TLC monitoring), cooled to rt and quenched with Na₂SO₄×10 H₂O (3 gr). Water (0.1 mL) and EtOAc (30 mL) were added and stirring was continued overnight. The suspension was filtered over Celite, the solvent was evaporated, and the residue was purified by column chromatography (CH₂Cl₂/MeOH/ammonia 3:1:0.1) and crystallization from MeCN yielded 65%) red-brown solid.

### Compounds #11039 and 11040: Chiral Purification of Compound 11006

The racemic mixture of Compound #11006 was later purified using chiral column to provide Compounds #11039 and 11040. The reaction scheme was:

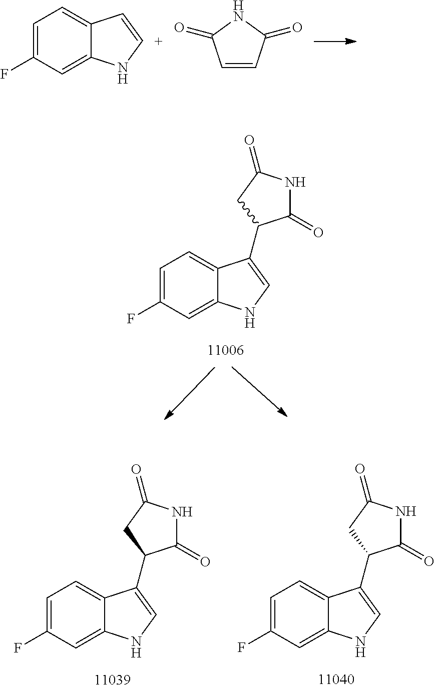 [SMILES: Fc1ccc2c(c1)N[C+]=C2 ; O=C1C=CC(=O)N1 ; O=C1CC(c2c[nH]c3cc(F)ccc23)C(=O)N1 ; O=C1C[C@H](c2c[nH]c3cc(F)ccc23)C(=O)N1 ; O=C1C[C@@H](c2c[nH]c3cc(F)ccc23)C(=O)N1]

### Example 68—Synthesis of Compound #11017-11019, 11021-11024, 11028, 11036-11038

The following compounds were prepared using the procedure described in Example 67. More specifically, the following scheme was used to produce the compounds.

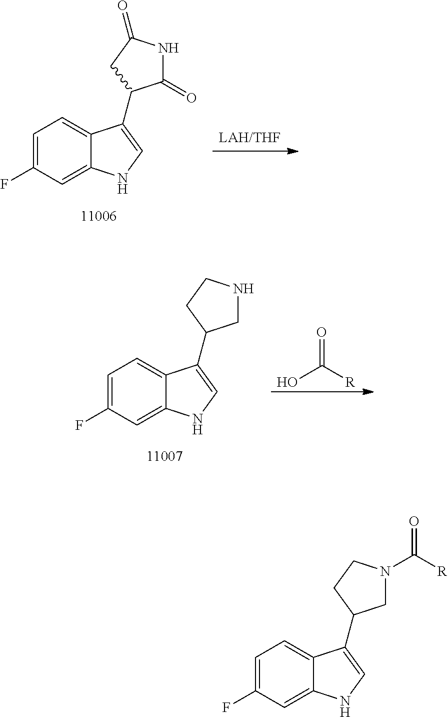

The specific reaction schemes were:

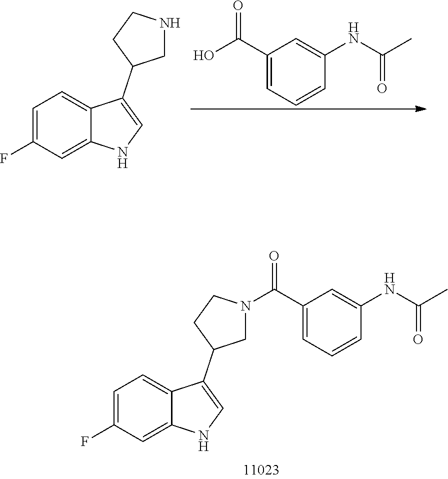

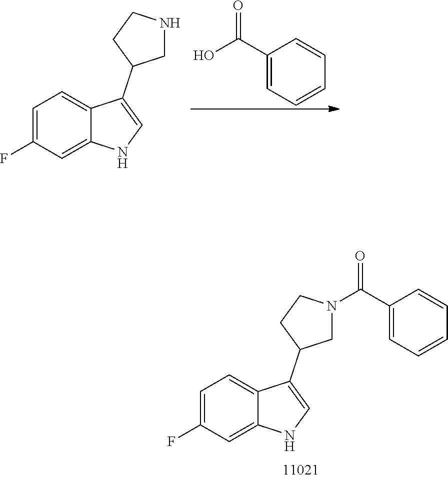

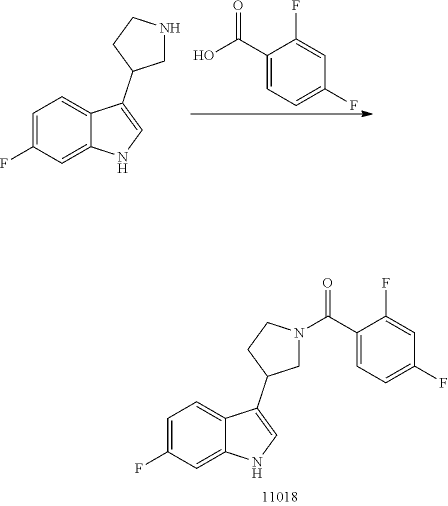

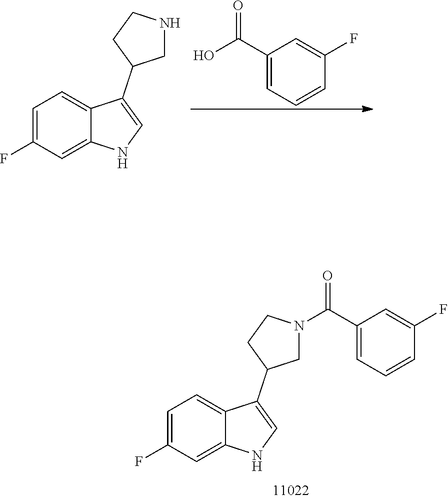

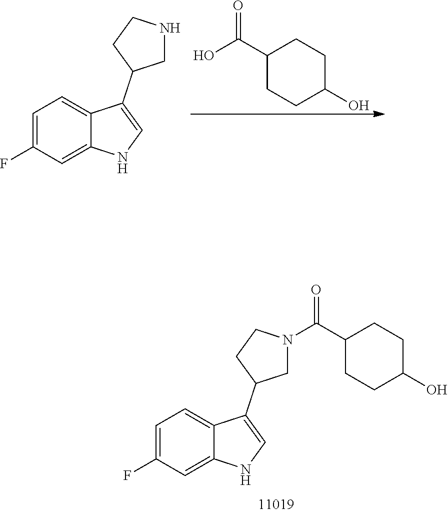

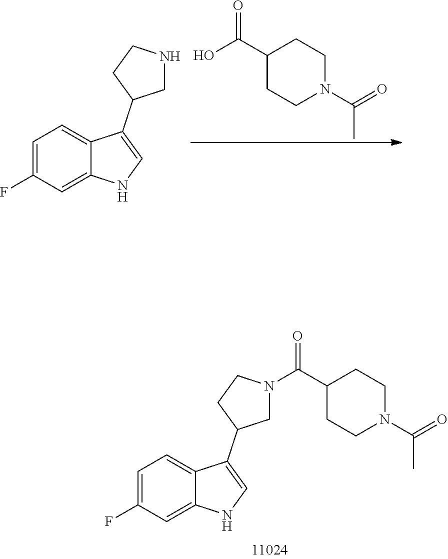

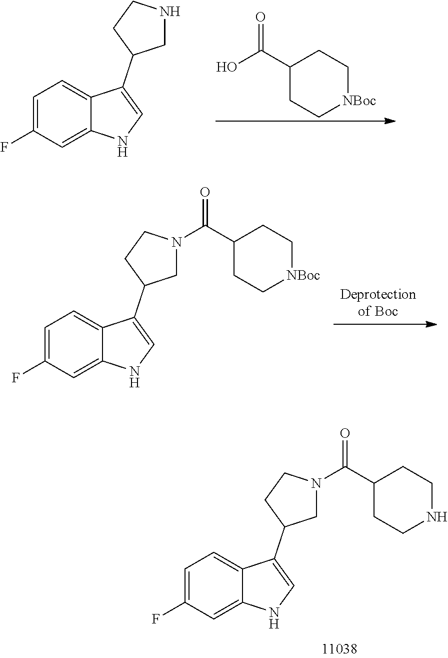 [SMILES: Fc1ccc2c(C3CCNC3)c[nH]c2c1 ; CC(C)(C)OC(=O)N1CCC(C(=O)O)CC1 ; CC(C)(C)OC(=O)N1CCC(C(=O)N2CCC(c3c[nH]c4cc(F)ccc34)C2)CC1 ; O=C(C1CCNCC1)N1CCC(c2c[nH]c3cc(F)ccc23)C1]

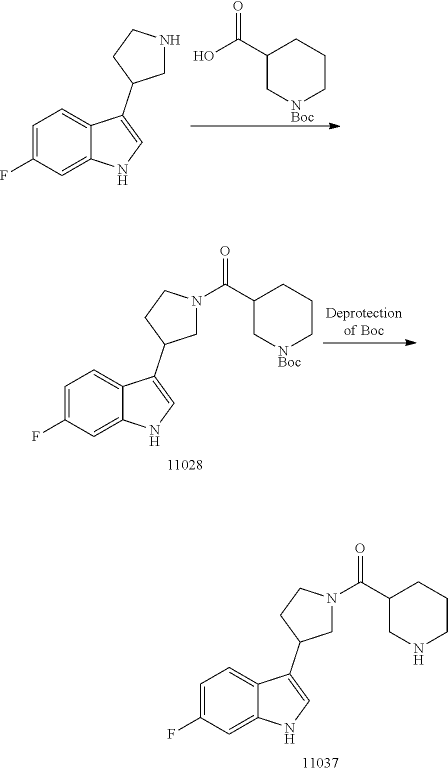 [SMILES: Fc1ccc2c(C3CCNC3)c[nH]c2c1 ; CC(C)(C)OC(=O)N1CCCC(C(=O)O)C1 ; CC(C)(C)OC(=O)N1CCCC(C(=O)N2CCC(c3c[nH]c4cc(F)ccc34)C2)C1 ; O=C(C1CCCNC1)N1CCC(c2c[nH]c3cc(F)ccc23)C1]

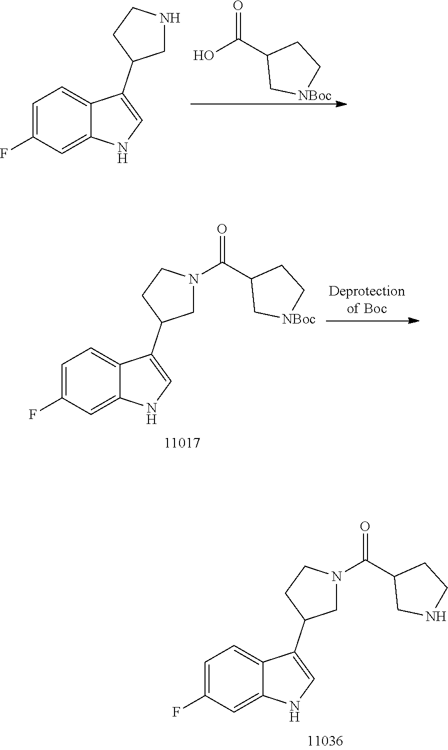

Coupling Reaction Used for the acid amine Coupling

To a round bottomed flask, 0.3 mmol of the corresponding acid, HATU (138.88 mg, 0.36 mmol) and 2 ml of DMF was added while stirring at 0° C. The solution was stirred at 0° C. for 5 min. Then DIEA (46.5 mg, 0.36 mmol was added to the mixture and continued stirring at 0° C. for another 5 min. Then Compound #11007 (43 mg, 0.3 mmol) was added and the solution was warmed to rt and stirred for more 18 hrs. Then the mixture was diluted with DCM (30 ml) and washed with HCL (15 ml, 1.0 N) and brine (20 ml), dried over sodium sulfate anhydrous. After the filtration and being concentrated in vacuum the residue was purified by column EtOAc and heptane to provide pure products.

### Deprotection of Boc for Preparing Compounds #11038, 11037 and 11017

Compounds #11038, 11037 and 11017 was prepared by the following method using their corresponding Boc protected amines. The Boc protected amines Compounds #11028, and 11017 was dissolved in a mixture of 1:1 DCM:TFA and stirred at room temperature for 1 hr. The volatile components were evaporated providing the corresponding amines which was later purified using silica in the DCM:Methanol:Ammonia (94:5:1) to give the pure product (yield 95-98%).

### Example 69—Synthesis of Compounds #11008 and 11014

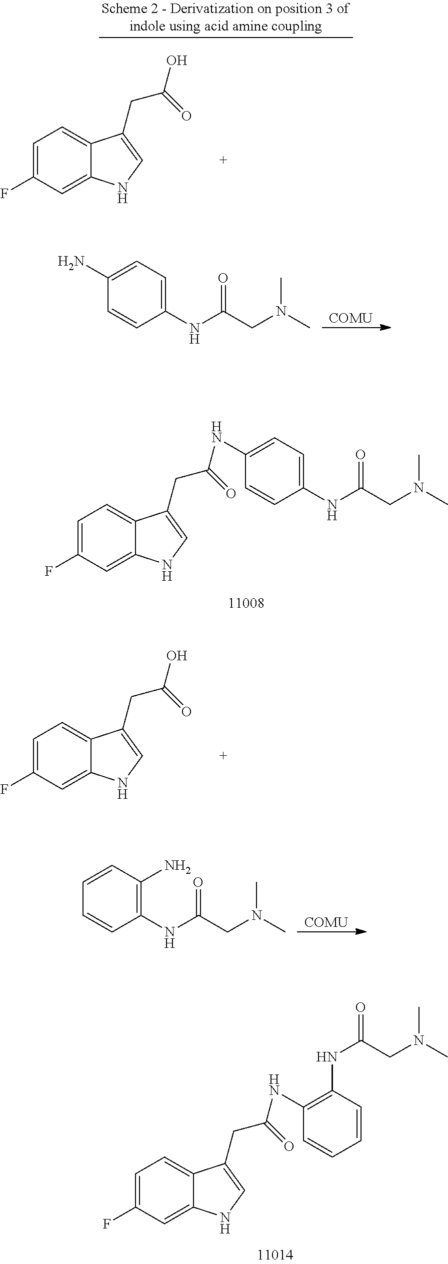

To a round bottomed flask, 2-(6-fluoro-1H-indol-3-yl) acetic acid (50 mg, 0.26 mmol), COMU (119 mg, 0.26 mmol), DIC (32.8 mg, 0.28 mmol) and 1.2 ml of DMF was added while stirring at 0° C. The solution was stirred at 0° C. for 5 min. Then DIEA (43 mg, 0.0338 mmol) was added to the mixture and continued stirring at 0° C. for another 5 min. Then substituted para-aniline (60 mg, 0.26 mmol) was added and the solution and kept for more 1 hr at 0° C. After 1 hr the solution was warmed to rt and stirred for more 18 hrs. The mixture was then diluted with DCM (30 ml) and washed with HCL (15 ml, 1.0 N) and brine (20 ml), and dried over sodium sulfate anhydrous. After filtration and concentration in vacuum, the residue was purified by reverse phase chromatography.

### Example 70—Synthesis of Compound #11011 and 11012

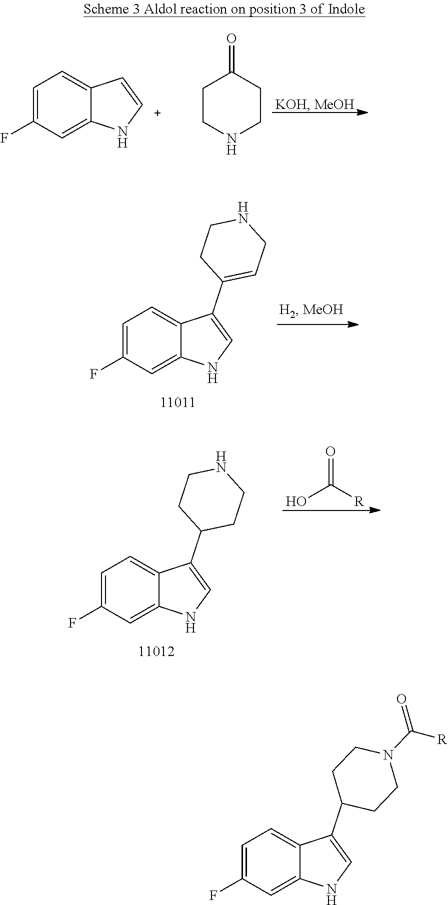

### Aldol Reaction/Procedure for the Preparation of Compound #11011

Indole (300.0 mg, 2.60 mmol) was dissolved in a solution of potassium hydroxide (770.6 mg, 13.8 mmol) in methanol (6.9 mL). 4-Piperidone monohydrate hydrochloride (1023 mg, 6.6 mmol) was added in one portion, and the mixture was heated to reflux for 5 h. Potassium chloride precipitated upon cooling to room temperature, and it was filtered off. The liquid phase was concentrated until only one-third of the liquid remained in the round bottom flask. Water was added, and a solid precipitated, which was filtered and washed with ethyl ether (63% yield) the product was used without purification for the next step.

### Hydrogenation Reaction/Procedure for the Preparation of Compound #11012

The reaction was setup in an oven-dried round bottom flask (RBF) with a three-way connector attached on the top. The connector was a three-way valve junction where allow switching from vacuum to nitrogen and/or hydrogen. The RBF was purged three times with nitrogen followed by vacuum to remove any trace quantities of oxygen and moisture. Then, 300 mg, 1.40 mmol, of 6-fluoro-3-(piperidin-4-yl)-1H-indole (Compound 11011) was introduced to this RBF under flow of nitrogen, followed by addition of 20 mL of dry methanol as a solvent. Then, 10% Pd/C (31 mg) as a catalyst was loaded in the reaction. Finally, the nitrogen balloon was replaced with a hydrogen balloon and the reaction was purged three times with hydrogen to ensure complete removal of N₂ gas (may acts as a diluent for hydrogen during the hydrogenation reaction). The hydrogen balloon was refreshed every 12 h for 36 h until the completion of the reaction. Then, the reaction mixture was filtered using a prepared celite plug. Then, the filtrate was concentrated using the rotary vaporizer. The resultant crude product was taken to the next step without any further purification. (yield: 99%).

### Example 71—Synthesis of Compounds #11017, 11020, 11025-11027, 11029, and 11046-11048

The following scheme was used to produce the compounds.

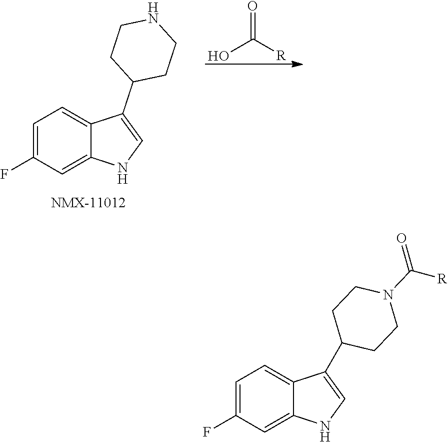

The specific reaction schemes were:

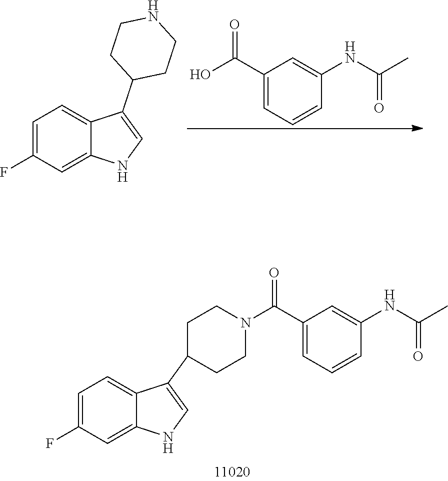

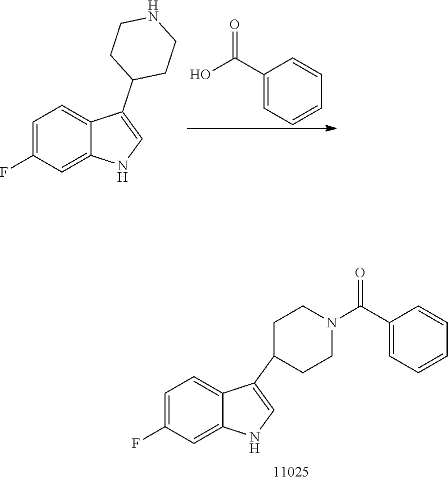

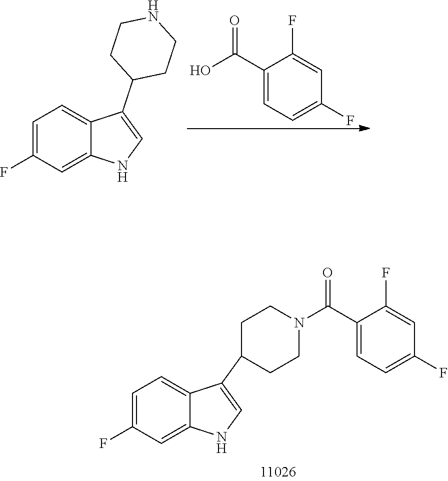

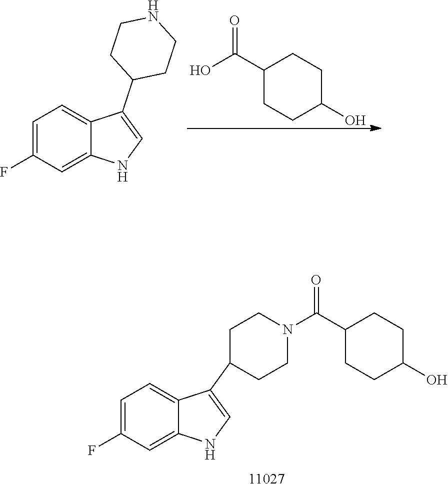

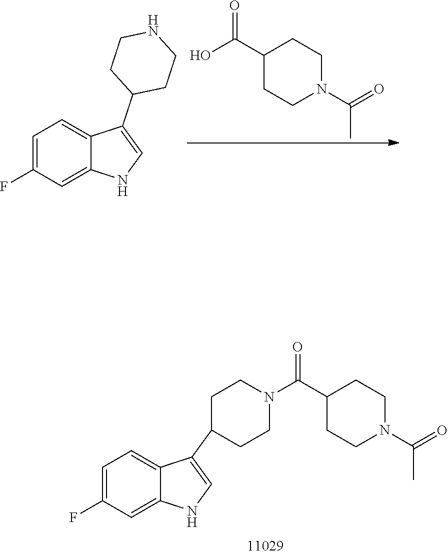

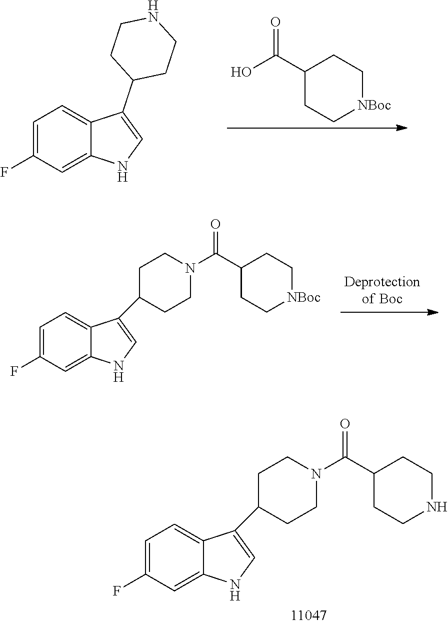 [SMILES: Fc1ccc2c(C3CCNCC3)c[nH]c2c1 ; CC(C)(C)OC(=O)N1CCC(C(=O)O)CC1 ; CC(C)(C)OC(=O)N1CCC(C(=O)N2CCC(c3c[nH]c4cc(F)ccc34)CC2)CC1 ; O=C(C1CCNCC1)N1CCC(c2c[nH]c3cc(F)ccc23)CC1]

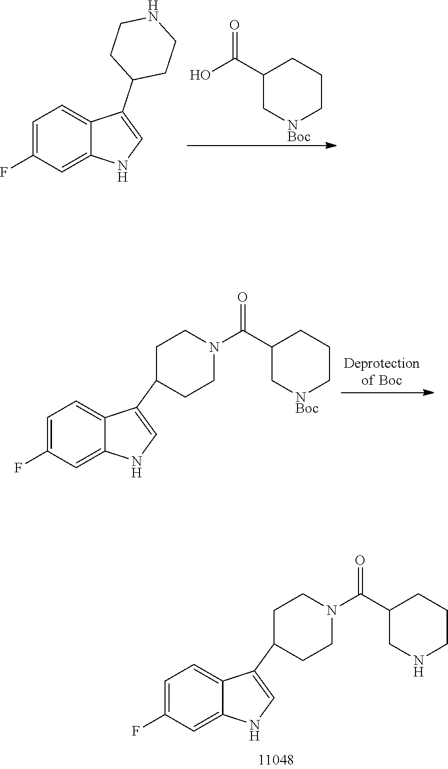

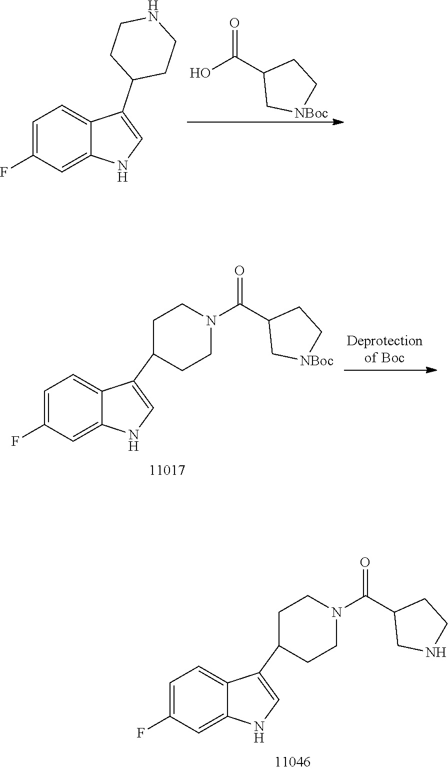

Coupling Reaction Used for the acid amine Coupling

To a round bottom flask, 0.3 mmol of the corresponding acid, HATU (138.88 mg, 0.36 mmol) and 2 ml of DMF was added while stirring at 0° C. The solution was stirred at 0° C. for 5 min. Then DIEA (46.5 mg, 0.36 mmol was added to the mixture and continued stirring at 0° C. for another 5 min. Then Compound #11012 (0.3 mmol) was added and the solution was warmed to rt and stirred for more 18 hrs. Then the mixture was diluted with DCM (30 ml) and washed with HCL (15 ml, 1.0 N) and brine (20 ml), dried over sodium sulfate anhydrous. After the filtration and being concentrated in vacuum the residue was purified by column EtOAc and heptane to provide pure products.

### Deprotection of Boc for Preparing Compound #11046, 11047 and 11048

Compounds #11046, 11047 and 11048 were prepared by the following method using their corresponding Boc protected amines. The Boc protected amines was dissolved in a mixture of 1:1 DCM:TFA and stirred at room temperature for 1 hr. The volatile components was evaporated after 1 hr providing the corresponding amines which was later purified using silica in the DCM:Methanol:Ammonia (94:5:1) to give the pure product (yield 95-98%).

### Example 72—Synthesis of Compounds #11041, 11042, 11043, 11052, and 11053

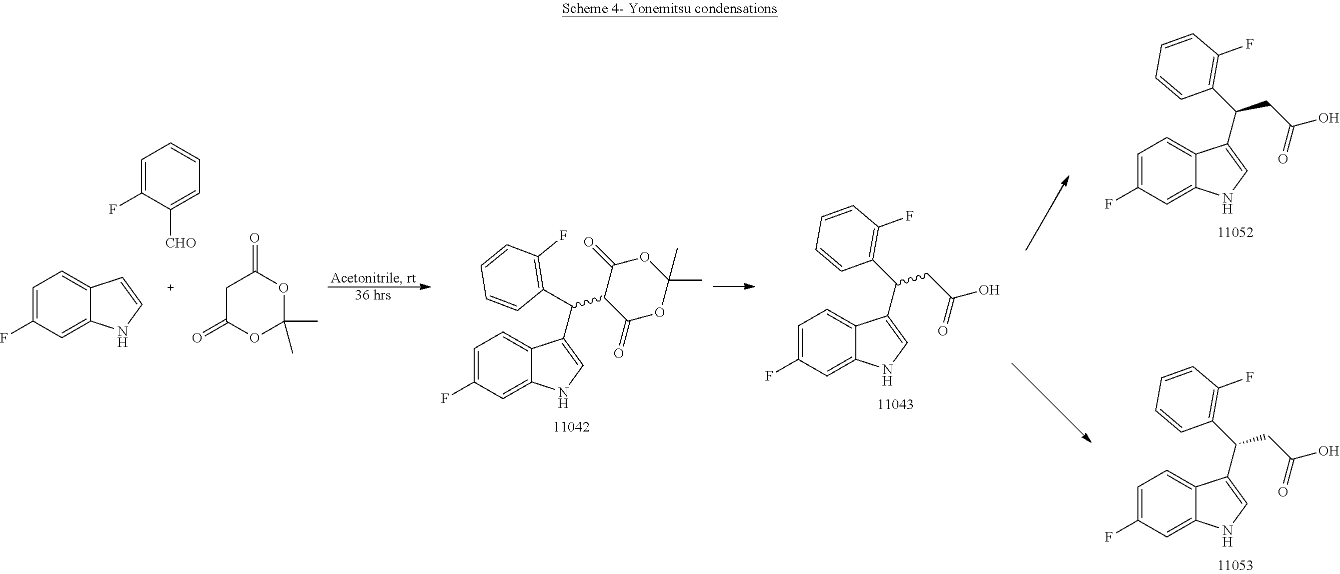

Yonemitsu Condensations Catalyzed by proline

To a solution of 2-fluorobenzaldehyde (183.68 mg, 1.48 mmol) in acetonitrile (20 ml) were added successively 6-fluoro-1H-indole 1 (100 mg, 0.74 mmol), Meldrum's acid (106.7 mg, 0.74 mmol) and L-proline (4 mg, 0.037 mmol). The reaction mixture was stirred at room temperature under nitrogen for 36 h, the solvent was evaporated, and the residue was purified by column chromatography (hexane-ethyl acetate 8:2) to afford the two enantiomers Compounds #11041 and 11042, 67%).

Hydrolysis of Meldrum's acid to acid

50 mg of Compound #11042 was dissolved in 4 ml mixture of water:pyridine (1:3) at room temperature. The reaction mixture was heated to reflux for 18 hrs. Then the reaction mixture was cooled down to room temperature. 15 ml of EtOAc was added to the mixture and extracted 2 times. The solution was dried over sodium sulfate anhydrous. After the filtration and being concentrated in vacuum the residue was purified by column EtOAc and heptane to provide pure product Compound #11043 (yield 95%).

### Hydrolysis of Meldrum's acid to ester

50 mg of Compound #11042 was dissolved in 4 ml mixture of EtOH:pyridine (1:3) at room temperature. The reaction mixture was heated to reflux for 18 hrs. Then the reaction mixture was cooled down to room temperature. 15 ml of EtOAc was added to the mixture and extracted 2 times. The solution was dried over sodium sulfate anhydrous. After the filtration and being concentrated in vacuum the residue was purified by column EtOAc and heptane to provide pure products Compound #11045 (yield 92%).

### Chiral Resolution of Compounds #11052 and 11053

The two isomers of Compound #11043 was separated using chiral column chromatography to provide enantiomeric pure compounds of Compounds #11052 and 11053.

### Example 73—Synthesis of Compounds #11054 to 11059

The reaction scheme was:

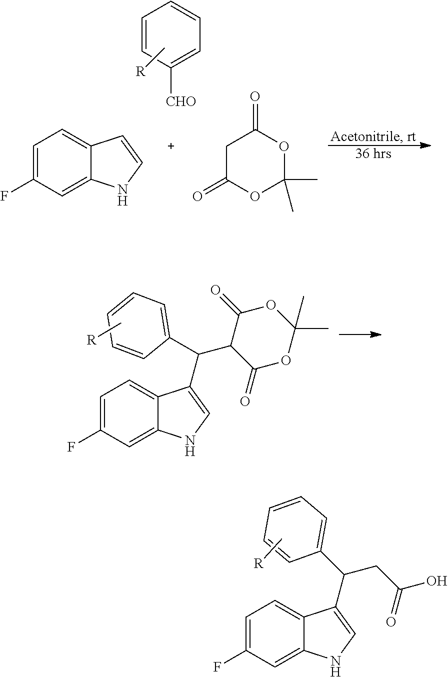

Yonemitsu Condensations Catalyzed by proline for the Derivatives (Compounds #11054-11059)

The following compounds were produced.

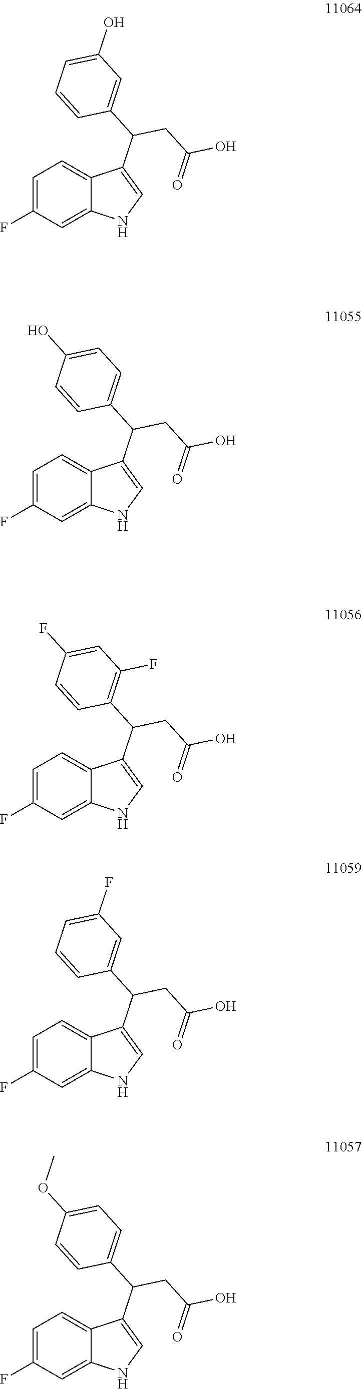 [SMILES: O=C(O)CC(c1cccc(O)c1)c1c[nH]c2cc(F)ccc12 ; O=C(O)CC(c1ccc(O)cc1)c1c[nH]c2cc(F)ccc12 ; O=C(O)CC(c1ccc(F)cc1F)c1c[nH]c2cc(F)ccc12 ; O=C(O)CC(c1cccc(F)c1)c1c[nH]c2cc(F)ccc12 ; COc1ccc(C(CC(=O)O)c2c[nH]c3cc(F)ccc23)cc1]

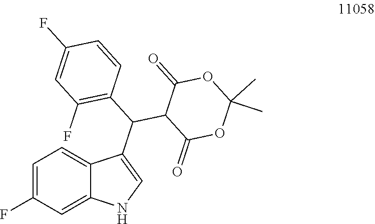 [SMILES: CC1(C)OC(=O)C(C(c2ccc(F)cc2F)c2c[nH]c3cc(F)ccc23)C(=O)O1]

To a solution of the corresponding benzaldehyde (1.48 mmol) in acetonitrile (20 ml-) were added successively 6-fluoro-1H-indole 1 (100 mg, 0.74 mmol), Meldrum's acid (106.7 mg, 0.74 mmol) and L-proline (4 mg, 0.037 mmol). The reaction mixture was stirred at room temperature under nitrogen for 36 h, the solvent was evaporated, and the residue was purified by column chromatography (hexane-ethyl acetate 8:2) to afford the corresponding product, 20-60%).

Hydrolysis of Meldrum's acid to acid

50 mg of Compound #11041 was dissolved in 4 ml mixture of water:pyridine (1:3) at room temperature. The reaction mixture was heated to reflux for 18 hrs. Then the reaction mixture was cooled down to room temperature. 15 ml of EtOAc was added to the mixture and extracted 2 times. The solution was dried over sodium sulfate anhydrous. After the filtration and being concentrated in vacuum the residue was purified by Isocratic chromatographic system by Biotage using 1:3:6 solution of Methanol:EtOAC:Toluene (yield 90%).

### Example 74—Synthesis of Compounds #11060 to 11063

The reaction scheme was:

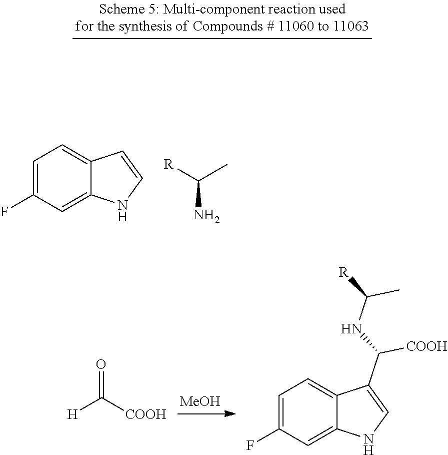

The following compounds were produced.

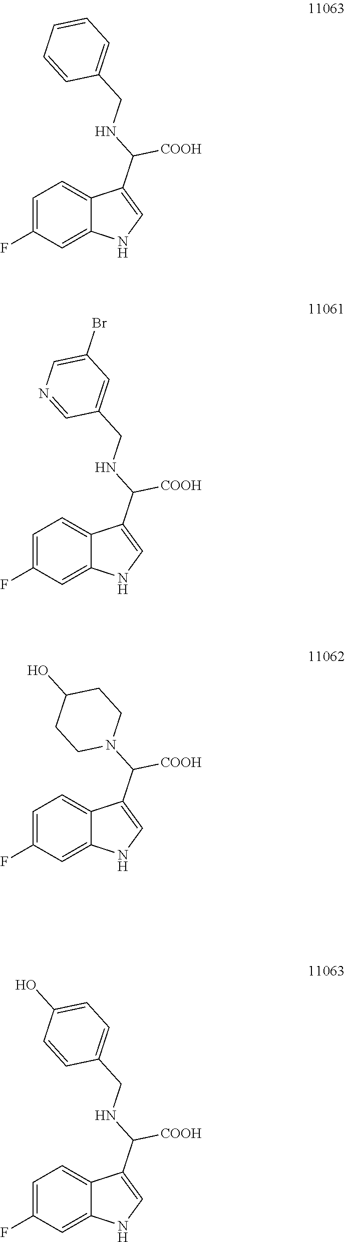 [SMILES: O=C(O)C(NCc1ccccc1)c1c[nH]c2cc(F)ccc12 ; O=C(O)C(NCc1cncc(Br)c1)c1c[nH]c2cc(F)ccc12 ; O=C(O)C(c1c[nH]c2cc(F)ccc12)N1CCC(O)CC1 ; O=C(O)C(NCc1ccc(O)cc1)c1c[nH]c2cc(F)ccc12]

To a stirred solution of 6-fluoro-1H-indole (5 mmol) and benzylamine (5.5 mmol) in 10 mL of dry methanol at 0° C. was added solution of glyoxylic acid monohydrate (506 mg; 5.5 mmol) dissolved in 10 mL of dry MeOH. The reaction mixture was stirred at room temperature for 1 hour. The precipitated product was filtered off, washed with MeOH and Et₂O and dried. The corresponding product was obtained as white solid (90-95%).

### Example 75—Synthesis of Compounds #11013, 11015, 11016, 11030-11033, and 11035

The reaction scheme was:

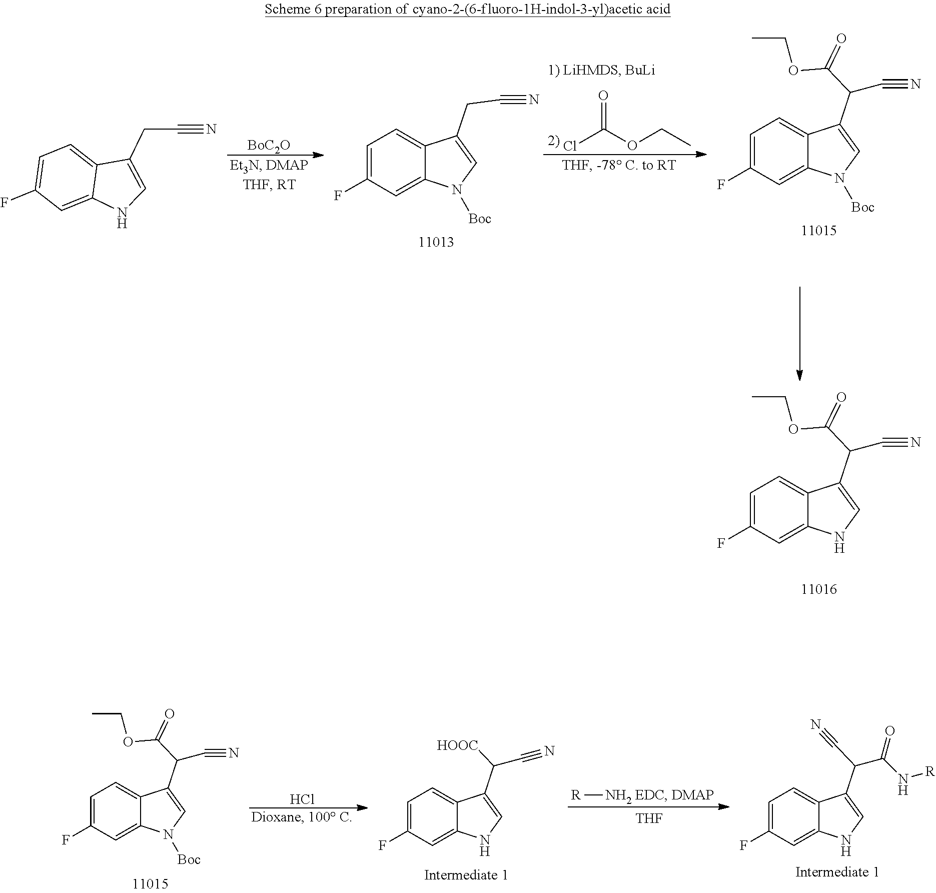

Boc Protection of 6-Fluoroindole acetonitrile: Preparation of Compound #11013

2-(6-fluoro-1H-indol-3-yl) acetonitrile (1.5 g, 8.612 mmol) was dissolved in THE (10.0 mL) and to the resulting solution was added Boc₂O (2.25 g, 10.334 mmol), DMAP (193.2 mg, 1.722 mmol) and 2.37 mL (12.92 mmol) of TEA. The reaction was stirred at room temperature for 1 hour, and then was diluted with EtOAc (80 mL). The organic solution was washed with saturated NaHCO₃ (80 mL) and brine (80 mL). The organic layer dried over Na₂SO₄ and concentrated to give crude product. The crude was purified by flash column to get titled compound as yellow solid. The product can be purified using column chromatography using 0 to 30% ethyl acetate:heptanes or crystallized with 10:90 Ethyl Acetate:Heptane. The product Compound #11013 is a fluffy white powder.

### Preparation of Compound #11015

Dissolved HMDS (1.12 mL, 5.658 mmol) in 3.0 ml of THE at −78° C. under inert atmosphere. Then, add BuLi (2.4 mL, 2.36M, 5.658 mmol) dropwise, and let the mixture stir for 20 minutes. Then, dissolve 2-(6-fluoro-1H-indol-3-yl) acetonitrile-BOC (0.7761 g, 2.829 mmol) 6.0 in mL of THE in a separate flask under inert atmosphere. Transfer this solution to the LiHMDS mixture dropwise. Let stir for 1.5 hours. Dilute ethyl chloroformate (323 uL, 3.395 mmol) in 11.0 mL of THE under inert atmosphere and add dropwise to the other flask containing LiHMDS and the indole. The mixture was stirred overnight. Dilute the reaction mixture with 20 mL saturated NH4Cl, and extract 2×20 mL with ethyl acetate. Then, wash with brine and dry over sodium sulfate. Filter and evaporate under reduced pressure. Purify with column with a 0 to 30% gradient Ethyl Acetate/Heptane to produce a yellow viscous oil (0.951 g, 97%).

### Preparation of Intermediate 1

Compound #11015 (2.236 g, 6.455 mmol) was dissolved in 40 mL of dioxane and 1 N hydrochloric acid (6.50 mL, 6.5 mmol) was added. The reaction mixture was refluxed at 100° C. overnight. The reaction mixture was evaporated under reduced pressure and was washed with 40 mL of 3% Sodium bicarbonate. Then, it was extracted with DCM 1×10 mL. This organic layer was disposed. Then, the aqueous layer was re-acidified with 12M HCl and a re-extracted with EtOAc 3×30 mL. The organic layer was dried over sodium sulfate and evaporated under reduced pressure. The product was a crystalline orange-white solid. Yield=1.086 g, 76.9%. The product was used without further purification.

### Reaction of Intermediate 1

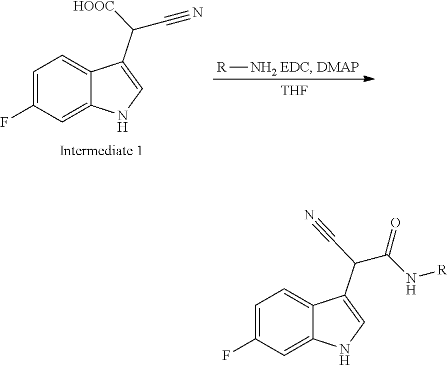

Dissolve the amine (0.1811 mmol) in 3 mL of THE and chill on an ice bath. Add intermediate 1 (0.2064 mmol) to the amine solution under N₂. Suspend EDC-HCl (0.2519 mmol) and DMAP (0.0241 mmol) in 6 mL THE and add dropwise. Stirred at room temperature overnight. Dilute with 20 mL of EtOAc and wash 2× with 10 mL water followed by 1× with 10 mL brine. Dry over sodium sulfate, filter and evaporate under reduced pressure. Purify with reverse phase chromatography. (Except intermediate 2, which was purified with normal phase). Yield=15-70%.

The following compounds were produced this way:

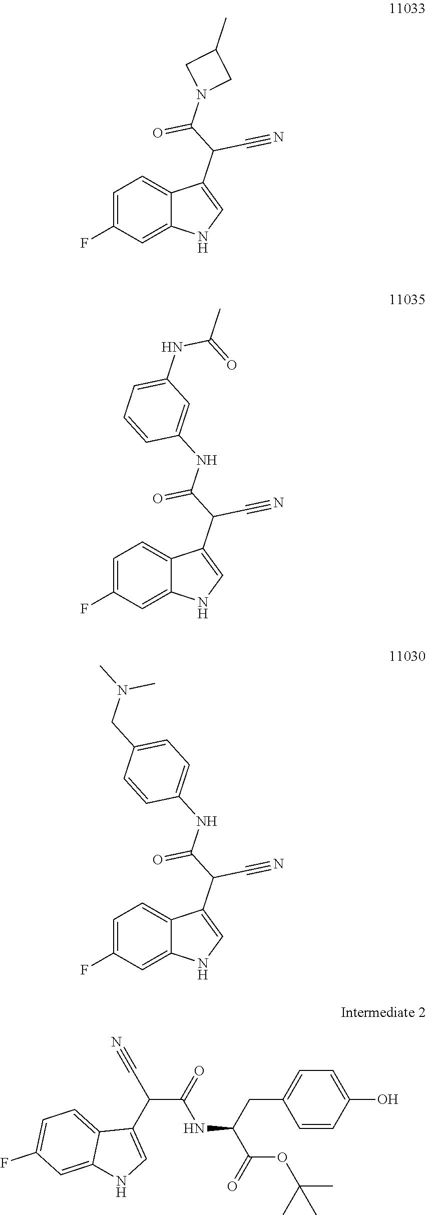 [SMILES: CC1CN(C(=O)C(C#N)c2c[nH]c3cc(F)ccc23)C1 ; CC(=O)Nc1cccc(NC(=O)C(C#N)c2c[nH]c3cc(F)ccc23)c1 ; CN(C)Cc1ccc(NC(=O)C(C#N)c2c[nH]c3cc(F)ccc23)cc1 ; CC(C)(C)OC(=O)[C@H](Cc1ccc(O)cc1)NC(=O)C(C#N)c1c[nH]c2cc(F)ccc12]

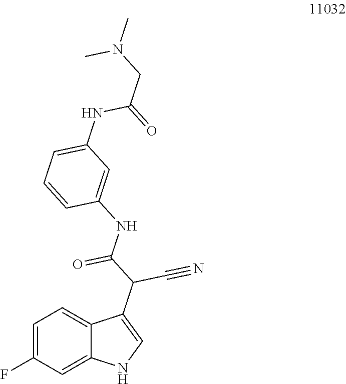 [SMILES: CN(C)CC(=O)Nc1cccc(NC(=O)C(C#N)c2c[nH]c3cc(F)ccc23)c1]

Preparation of Compound #11031 from Intermediate 2

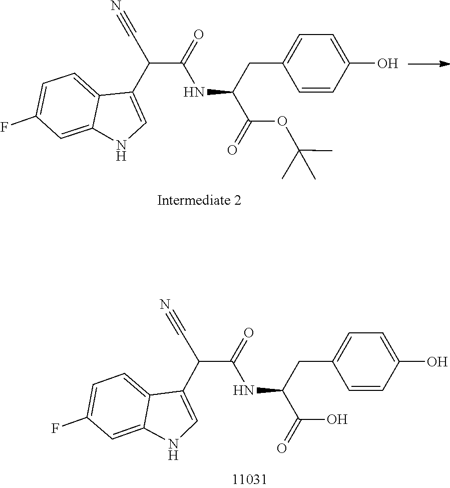 [SMILES: CC(C)(C)OC(=O)[C@H](Cc1ccc(O)cc1)NC(=O)C(C#N)c1c[nH]c2cc(F)ccc12 ; N#CC(C(=O)N[C@@H](Cc1ccc(O)cc1)C(=O)O)c1c[nH]c2cc(F)ccc12]

Dissolved tert-butyl (2-cyano-2-(6-fluoro-1H-indol-3-yl)acetyl)-L-tyrosinate (0.0231 g, 0.0528 mmol) in 1 mL DCM and then add 1 mL of trifluoroacetic acid. Stirred at room temperature for 2 hours. Then, evaporate the organic layer off. Re-dissolved in 3 mL DCM and evaporate under reduced pressure. Finally, dissolve in 3 mL of EtOAc and evaporate again. Purify with reverse phase chromatography (0.017 mg, 84.5% yield). **Due to the chirality of L-tyrosine there are diastereomers.

Preparation of Compound #11016 from Compound #11015

tert-butyl 3-(cyanomethyl)-6-fluoro-1H-indole-1-carboxylate (60 mg, Compound 11015) dissolved in 6 ml of TFA:DCM and stirred for 3 hrs. The reaction mixture was evaporated after 3 hrs providing the corresponding amines which was later purified using reverse phase chromatography (yield 95-98%).

### Example 76—Compounds #11001-11005 and 11034

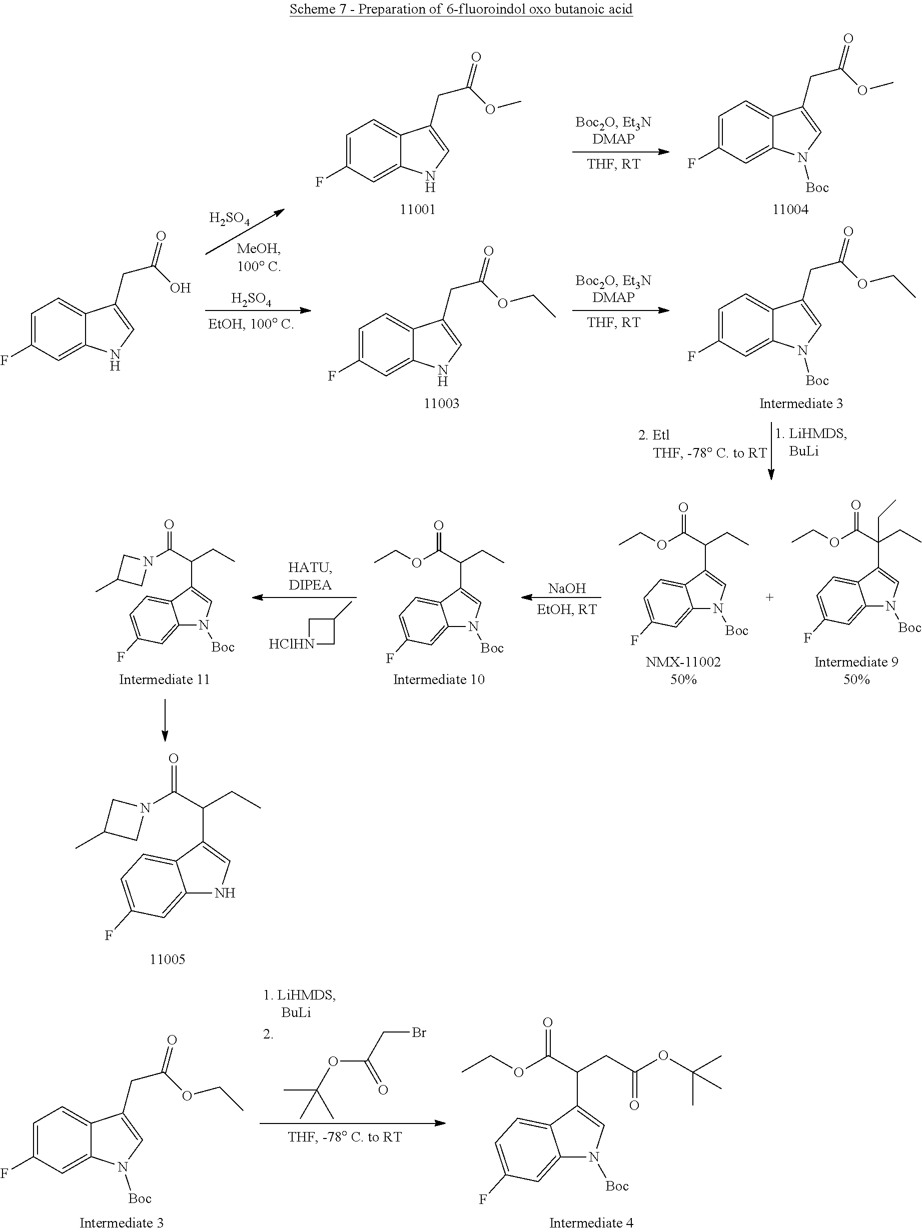

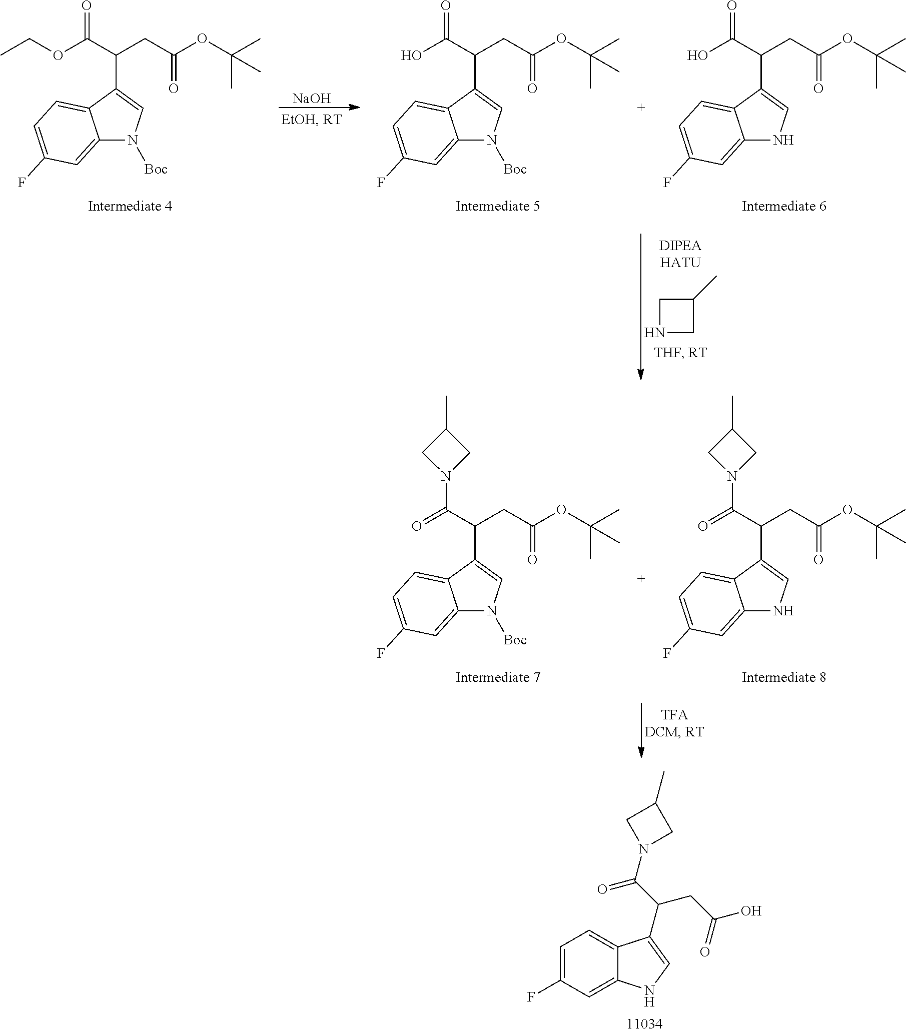

### Preparation of Compound #11001

2-(6-fluoro-1H-indol-3-yl) acetic acid (1.00 g, 5.177 mmol) was dissolved in methanol (15 mL) and concentrated sulfuric acid (1 mL). The solution was refluxed for 4.5 hours. Then, the mixture was cooled down to 0° C. in an ice bath before neutralization with 2 N sodium hydroxide solution. The organics were evaporated under reduced pressure and the mixture was partitioned between dichloromethane (50 mL) and water (20 mL). The organic layer was separated and washed with brine (20 mL) and dried over sodium sulfate. After filtration, the organic layer was evaporated under reduced pressure affording ethyl 2-(6-fluoro-1H-indol-3-yl) acetate as a yellow oil (1.071 g, 99% yield). The product was used in the following step without further purification.

### Preparation of Compound #11003

2-(6-fluoro-1H-indol-3-yl) acetic acid (1.00 g, 5.177 mmol) was dissolved in ethanol (15 mL) and concentrated sulfuric acid (1 mL). The solution was refluxed for 4.5 hours. Then, the mixture was cooled down to 0° C. in an ice bath before neutralization with 2 N sodium hydroxide solution. The organics were evaporated under reduced pressure and the mixture was partitioned between dichloromethane (50 mL) and water (20 mL). The organic layer was separated and washed with brine (20 mL) and dried over sodium sulfate. After filtration, the organic layer was evaporated under reduced pressure affording ethyl 2-(6-fluoro-1H-indol-3-yl) acetate as a yellow oil (1.071 g, 99% yield). The product was used in the following step without further purification.

### Preparation of Compound #11004

Methyl 2-(6-fluoro-1H-indol-3-yl) acetate (1.14 g, 5.502 mmol). Boc₂O (1.44 mg, 6.602 mmol), DMAP (0.0108 mg, 0.097 mmol), and triethylamine (1.15 mL, 8.253 mmol) was dissolved in THE (11 mL). The reaction was stirred at room temperature for 2 hours, and then was diluted with EtOAc (300 mL). The organic solution was washed with saturated NaHCO₃ (150 mL) and brine (150 mL), dried over Na₂SO₄ and concentrated to give crude product, which was purified by flash column to get titled compound as yellow oil.

### Preparation of Intermediate 3

Ethyl 2-(6-fluoro-1H-indol-3-yl) acetate (1.14 g, 5.502 mmol). Boc₂0 (1.44 mg, 6.602 mmol), DMAP (0.0108 mg, 0.097 mmol), and triethylamine (1.15 mL, 8.253 mmol) was dissolved in THE (11 mL). The reaction was stirred at room temperature for 2 hours, and then was diluted with EtOAc (300 mL). The organic solution was washed with saturated NaHCO₃ (150 mL) and brine (150 mL), dried over Na₂SO₄ and concentrated to give crude product, which was purified by flash column to get titled compound as yellow oil.

### Preparation of Intermediate 4

Butyl lithium (1183 μL, 2.5 M, 2.956 mmol) was added dropwise to a flask at −78° C. containing HMDS (743μ, 3.548 mmol) in 3.0 mL THE under N₂. The solution was left stirring for 20 minutes at −78° C. The Boc-Ethyl 2-(6-fluoro-1H-indol-3-yl) acetate was dissolved in 4.0 mL of dry THE and added dropwise and was left to stir for 1.5 hours at −78° C. Next, Tert-butyl bromo acetate (517 μL, 3.548 mmol) was dissolved in 5.0 mL dry THE and added dropwise. The reaction was stirred for an hour before letting it come to room temperature naturally. The mixture was left to stir overnight. The reaction was quenched with 10 mL saturated NH₄Cl and then extracted with EtOAc (50 mL). The organic layer was washed with brine (30 mL) and dried over Na₂SO₄. The organic layer was filtered and evaporated under reduced pressure. The product was further purified by 0-20% Ethyl acetate/Heptanes column chromatography gradient. The product was a slightly yellow, clear oil (1.234 g, 97% yield).

### Preparation of Intermediate 9 and Compound #11002

Butyl lithium (1183 μL, 2.5 M, 2.956 mmol) was added dropwise to a flask at −78° C. containing HMDS (743μ, 3.548 mmol) in 3.0 mL THE under N₂. The solution was left stirring for 20 minutes at −78° C. The Boc-Ethyl 2-(6-fluoro-1H-indol-3-yl) acetate was dissolved in 4.0 mL of dry THE and added dropwise and was left to stir for 1.5 hours at −78° C. Next, ethyl iodate (3.548 mmol) was dissolved in 5.0 mL dry THE and added dropwise. The reaction was stirred for an hour before letting it come to room temperature naturally. The mixture was left to stir overnight. The reaction was quenched with 10 mL saturated NH₄Cl and then extracted with EtOAc (50 mL). The organic layer was washed with brine (30 mL) and dried over Na₂SO₄. The organic layer was filtered and evaporated under reduced pressure. The product was further purified by 0-20% Ethyl acetate/Heptanes column chromatography gradient.

### Preparation of Intermediate 5, 6 and 10

Dissolved the corresponding indole (0.0823 mmoL) in 7 mL of EtOH and vortexed to insure full solubilization. Then, add 1.2 mL of KOH (1M) and let stir overnight at room temperature. Evaporate organic layer and extract 2 times with 15 mL of DCM. Then, wash the organic layer with 60 mL H₂O. Re-acidify the aqueous layer using 3M HCl, and extract 3× with 15 mL EtOAc. Keep the second organic extraction and dry over sodium sulfate and dry under reduced pressure. The product is clear oil and contains both the Boc and the deprotected versions which was used in the following step without further purification. (20-30% yield).

### Preparation of Intermediate 7, 8 and Compound #11005

Dissolved the mixture of intermediate 5 and 6 (0.053 g, 0.1448 mmol) in 0.5 mL DMF and added 40 μL (0.2985 mmol) of triethyl amine to a 5 mL round-bottom flask. Let stir for 5 minutes. Then, add HATU (0.055 g, 0.1448 mmol). In another flask, dissolve azetidine (0.0116 g, 0.1448 mmol) in 0.5 mL DMF and add 40 μL (0.2985 mmol) of triethylamine. Add this solution dropwise to the flask containing the mixture of intermediate 5 and 6 and let stir for 3 hours at room temperature. Dilute mixture with EtOAc and wash 5× with 5 mL of brine. Then, wash 2× with 10 mL HCl, followed by 2× with 10 mL brine. Dry over sodium sulfate, filter and evaporate under reduced pressure. The product is a clear oil at room temperature but crystallizes at 0° C. Used in the next step without further purification. (67.1 mg, 97% yield).

### Preparation of the Intermediate 11

Dissolved the intermediate 10 (0.1448 mmol) in 0.5 mL DMF and added 40 μL (0.2985 mmol) of triethyl amine to a 5 mL round-bottom flask. Let stir for 5 minutes. Then, add HATU (0.055 g, 0.1448 mmol). In another flask, dissolve azetidine (0.0116 g, 0.1448 mmol) in 0.5 mL DMF and add 40 μL (0.2985 mmol) of triethylamine. Add this solution dropwise to the flask containing intermediate 10 and let stir for 3 hours at room temperature. Dilute mixture with EtOAc and wash 5× with 5 mL of brine. Then, wash 2× with 10 mL HCl, followed by 2× with 10 mL brine. Dry over sodium sulfate, filter and evaporate under reduced pressure. Used in the next step without further purification.

### Preparation of the Compounds #11005 and 11034

Dissolve 0.0671 g (0.1538 mmol) of the corresponding Boc protected indole in 6 mL of TFA:DCM 50:50 and stir the reaction mixture for 4 hours. Quench the reaction with 10 mL of saturated sodium bicarbonate. Extract 1× with 10 mL of dichloromethane. Then, acidify the aqueous layer with 3M HCl, and extra 3×10 mL with DCM, keep this organic layer separate from the previous extraction. Wash this organic layer 1×10 mL of brine. Dry over sodium sulfate, filter and evaporate under reduced pressure. Purify with reverse phase chromatography.

### Example 76—Compound #11050

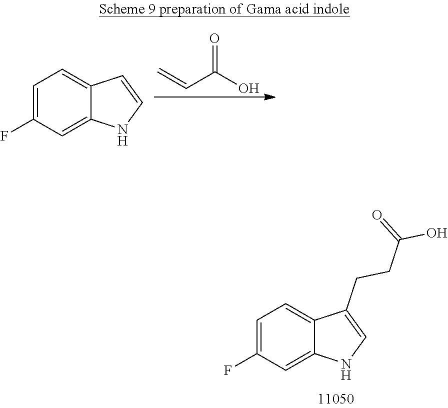

6-fluoro-1H-indole (1.5 mmol) was dissolved in acetic acid (1.8 mL). Acrylic acid (2.78 mmoles 0.2 mL) and acetic acid anhydride (3.18 mmoles 0.3 mL) were added, and the reaction mixture was heated to 95° C. for 24 hours. The reaction was then cooled, which was then extracted with ethyl acetate and 4N NaOH (aq.). The aqueous layer was made acidic with 6N HCl (aq.) to pH 2 and then extracted with ethyl acetate. The combined organics were dried and concentrated in vacuo. The residue was purified by Biotage on silica gel with 10% MeOH/DCM to afford corresponding product.

3. 11.9 mg (3.8%)

### Example 77—Compound #11066

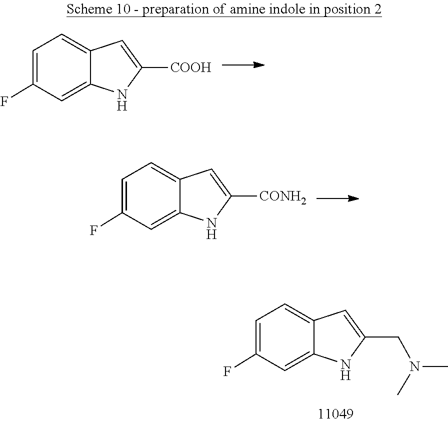

### Coupling Reaction

1a. To a stirring solution of NH2·HCl (6.66 mmoles, 555 mg) in dry DMF (5 mL) at room temperature under N₂, was added Et₃N (20.1 mmoles, 2.03 g). The mixture was stirred for 5 min. Then 6-fluoro-1H-indole-2-carboxylic acid (3.35 mmoles, 600 mg) and HATU (5.02 mmoles, 1.91 g) were added. The reaction was stirred for 16 h. The reaction was poured in a small amount of ice and kept in fridge for 1 h. Finally, the solid was isolated by filtration, washed with water and dried with EtO₂.

### Hydrogenation Step

To a suspension of LiAlH4 (9.27 mmoles, 351 mg) in dry THE at 0° C., was added dropwise a solution of the crude 6-fluoro-1H-indole-2-carboxamide (1.07 mmoles, 200 mg) in dry THF. The mixture was refluxed at 78° C. with stirring for 16 h. Then the reaction was cooled at room temperature and in ice water bath. Then water (0.4 mL), 15% NaOH (0.8 mL) and water (0.4 mL) were successively added. The obtained solid was filtrated. The solution was extracted by EtOAc, dried with Na₂SO₄ and concentered in vacuo. The product was finally purified with 10% MeOH/DCM giving pure product.

### Preparation of Compound #11051

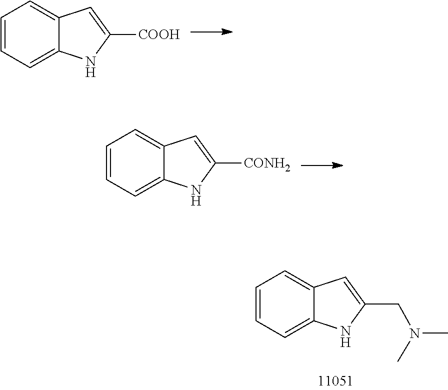

To a stirring solution of NH2·HCl (6.21 mmoles, 516 mg) in dry DMF (5 mL) at room temperature under N₂, was added Et₃N (18.6 mmoles, 1.88 g). The mixture was stirred for 5 min. Then 1H-indole-2-carboxylic acid (3.10 mmoles, 500 mg) and HATU (4.65 mmoles, 1.77 g) were added. The reaction was stirred at rt for 16 h. The reaction was poured in a small amount of ice and kept in fridge for 1 h. Finally, the solid was isolated by filtration, washed with water and dried with EtO₂.

To a suspension of LiAlH4 (9.27 mmoles, 351 mg) in dry THE at 0° C., was added dropwise a solution of the crude 6-fluoro-1H-indole-2-carboxamide (1.07 mmoles,) in dry THF. The mixture was refluxed at 78° C. with stirring for 16 h. Then the reaction was cooled at room temperature and in ice water bath. Then water (0.4 mL), 15% NaOH (0.8 mL) and water (0.4 mL) were successively added. The obtained solid was filtrated. The solution was extracted by EtOAc, dried with Na₂SO₄ and concentrated in vacuo. The product was finally purified with 10% MeOH/DCM giving pure product.

### Fragment Merging of 11043 and 11049 to Result in Preparation of Compound 11066

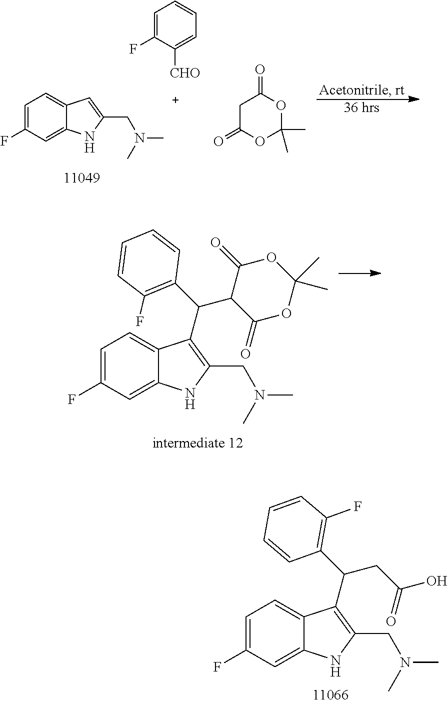

To a solution of the corresponding 2-fluorobenzaldehyde (1.48 mmol) in acetonitrile (20 mL) were added successively Compound #11049 (0.74 mmol), Meldrum's acid (106.7 mg, 0.74 mmol) and L-proline (4 mg, 0.037 mmol). The reaction mixture was stirred at room temperature under nitrogen for 36 h, the solvent was evaporated and the residue was purified by column chromatography (hexane-ethyl acetate 8:2) to afford the corresponding product, 20-60%).

Hydrolysis of Meldrum's acid to acid Compound 11066

50 mg of the intermediate 12 was dissolved in 4 ml mixture of water:pyridine (1:3) at room temperature. The reaction mixture was heated to reflux for 18 hrs. Then the reaction mixture was cooled down to room temperature. 15 ml of EtOAc was added to the mixture and extracted 2 times. The solution was dried over sodium sulfate anhydrous. After the filtration and being concentrated in vacuo the residue was purified by Isocratic chromatographic system by Biotage using 1:3:6 solution of Methanol:EtOAC:Toluene (yield 85%).

### Example 78—Compounds #11067, 11068, 11070, 11071, 11072 and 11073

Step I: General Procedure for the Preparation of Indole carboxylic acids 4 (Intermediates for Compounds #11067, 11068, 11070, 11071, 11072 and 11073).

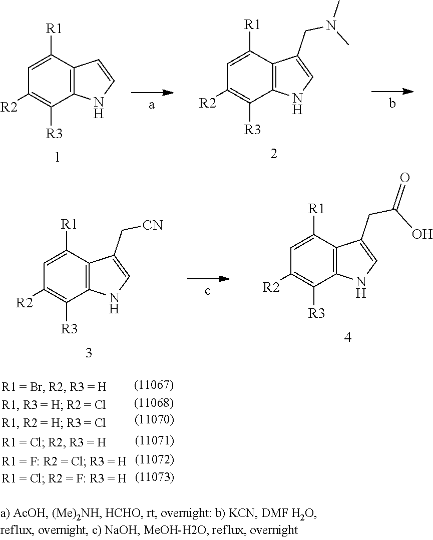

Preparation of indole dimethylamine Compounds—(1H-indol-3-yl)-N, N-dimethyl methanamine 2 (Example shown for Compound #11070 when R1, R2═H; R3═Cl)

The solution of 40 percent aq. dimethylamine (8.74 mmol, 1.2 eq) was cooled to 5° C., and glacial acetic acid (10.5 mmol) was added dropwise while maintaining the temperature at −10° C. After stirring for 20 minutes, 37 percent aqueous formaldehyde (8.74 mmol, 1 eqv) was slowly added to above solution while keeping the temperature between 0˜10° C., followed by addition of 7-chloro-indole 1 (7.40 mmol). The reaction was exothermic and reached a final temperature ˜40° C., and it was then cooled down to ˜20° C. The reaction solution was then slowly added to 16 mL of aqueous NaOH solution (3M). The suspension was stirred about 30 minutes, and then collected by filtration. The cake was rinsed with water (5 mL×2) and dried to afford compound 2 as a yellow solid in 96% yield (overall yields vary from 95 to 99%) used directly for the next step.

Preparation of indole acetonitrile Compounds—(1H-indol-3-yl) acetonitrile 3 (Example shown for Compound #11070 with R1, R2═H; R3═Cl)

The solution of (7-chloro-1H-indol-3-ylmethyl)-dimethylamine (2) (6 mmol), KCN (10.29 mmol, 1.72 eqv) in DMF (4 mL) and water (2 mL) were heated to 105° C. for 10 hours. After which period, the reaction mixture was cooled down to 25° C., water (14.5 mL) and toluene (8 mL) were added to the mixture and stirred for 3 hours. The organic and aqueous layers were separated. The organic layer was washed with aqueous sodium bicarbonate (8 mL) and brine (8 mL), dried over sodium sulfate. After filtration and concentrated, the residue was purified with flash column on silica gel to get desired product 3 as yellow oil (45-53% yield) used directly for the next step.

Preparation of indole acetic acid Compounds—(1H-indol-3-yl) acetic acid 4 (Example shown for Compound #11070 with R1, R2═H; R3═Cl)

The mixture of (7-chloro-1H-indol-3-yl) acetonitrile (3) (1.15 mmol), sodium hydroxide (6.50 mmol, 5.7 eq), methanol (1.5 mL) and water (4.5 mL) was stirred at 100° C. overnight. Then, the reaction was cooled to 0° C. and treated with 6 N aqueous solution of HCl to pH-1. The solid formed was collected by filtration, which was then washed twice with water and dried to give title compound 4 (Compound #11069) as a solid (50-69% yield).

For Compound #11069; when R₃═Cl, ¹H NMR (600 MHz, DMSO-d₆) δ 11.26 (s, 1H), 7.50-7.45 (m, 1H), 7.29-7.28 (m, 1H), 7.15 (dd, J=10.2, 2.3 Hz, 1H), 7.01-6.98 (m, 1H), 3.65 (s, 2H).

Step II: General Procedure of indole amide Coupling: For Compounds #11067, 11068, 11070, 11071, 11072 and 11073.

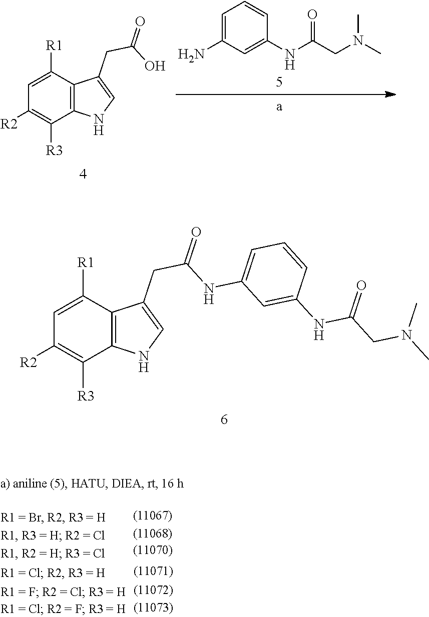

The solution of (1H-indol-3-yl) acetic acid 4 (0.52 mmol), N-(3-aminophenyl)-2-(dimethylamino)acetamide dihydrochloride (5) (152 mg, 0.57 mmol), HATU (216.5 mg, 0.57 mmol) and DIEA (595 μL, 3.42 mmol) in DCM (8 mL) was stirred at room temperature for 16 hours. After which, the mixture was diluted with dichloromethane (30 mL), washed with NaHCO₃ (2×20 ml) and brine (20 mL), dried over sodium sulfate. After filtration and concentration, the residue was purified by flash column on reverse phase to obtain desired product 6 as white powder (20-34% yield). Data on final compounds are shown in Table 1.

### Example 79—NMR and LCMS Analysis of the Compounds of Examples 67 to 77

TABLE 20

| | Compound | | | |
|---|---|---|---|---|
| Structure | # | ¹H NMR | LC-MS | |
| 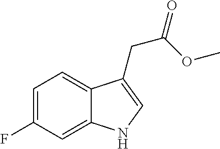 | 11001 | ¹H-NMR (600 MHz, DMSO-d6): 3.37 (s, 3H), 3.74 (s, 2H), 6.85 (m, 1H), 7.13 (1H, dd, J = 10.11, 2.31), 7.25 (1H, d, J = 2.31), 7.47 (1H, dd, J = 8.65, 5.46), 11.02 (s, 1H) | MS (ESI+) m/z 208.2 (M + H)⁺. | |
| 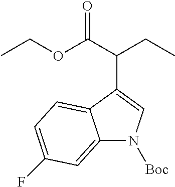 | 11002 | | Did not ionize | |
| 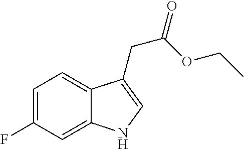 | 11003 | ¹H-NMR (600 MHz, DMSO-d6): 1.18 (3H, t, J = 7.11 Hz), 4.07 (2H, q, J = 7.11 Hz), 6.83 (1H, dt, J = 2.34, 14.10 Hz), 7.10 (1H, dd, J = 2.31, 10.09 Hz), 7.21 (1H, d, J = 2.30 Hz), 7.45 (1H, q, J = 4.71 Hz), 10.97 (1H, s). | MS (ESI+) m/z 202.2 (M + H)⁺. | |
| 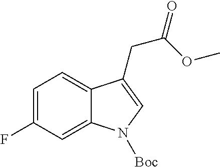 [SMILES: COC(=O)Cc1cn(C(=O)OC(C)(C)C)c2cc(F)ccc12] | 11004 | | Did not ionize | |
| 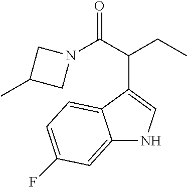 [SMILES: CCC(C(=O)N1CC(C)C1)c1c[nH]c2cc(F)ccc12] | 11005 | | | |
| 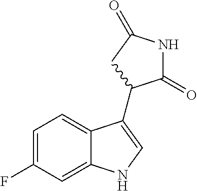 [SMILES: O=C1CC(c2c[nH]c3cc(F)ccc23)C(=O)N1] | 11006 | ¹H-NMR (600 MHz, DMSO-d6): 2.77 (1 H, d.d, J = 18.71, 5.40 Hz), 3.17 (1H, d.d., J = 18.71, 9.31 Hz), 4.34 (1H, d.d., J = 5.40, 9.31 Hz), 6.87 (1H, m), 7.15 (1H, d.d., J = 10.13, 2.48), 7.33, (1H, d, J = 2.48), 7.43 (1H, d.d., J = 5.27, 8.69), 11.29 (1H, s), 11.10 (1H, s). | MS (ESI+) m/z 233.07 (M + H)⁺. | |
| 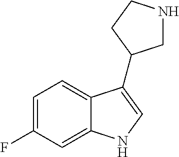 [SMILES: Fc1ccc2c(C3CCNC3)c[nH]c2c1] | 11007 | ¹H-NMR (600 MHz, (CD₃)₂CO, Acetone): 1.92 (1H m, 2.23 (1H, m), 2.74 (1H, t, J = 8.22), 2.87 (1H, m), 2.91 (1H, m), 3.21 (1H, t, J = 8.22), 3.49 (1H, d.t., J = 9.41, 8.07), 6.80 (1H, m), 7.10 (1H, dd J = 10.08, 2.24), 7.16 (1H, S), 7.65 (1H, dd, J = 9.16, 5.48) | MS (ESI+) m/z 205.12 (M + H)⁺. | |
| 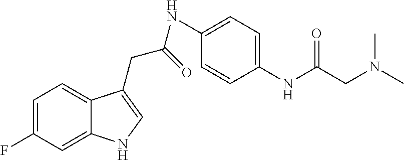 [SMILES: CN(C)CC(=O)Nc1ccc(NC(=O)Cc2c[nH]c3cc(F)ccc23)cc1] | 11008 | ¹H-NMR (600 MHz, DMSO-d6): 2.84 (6H, s), 3.69 (2H, s), 4.06 (2H, s), 6.54 (1H, s), 6.84 (1H, m), 7.12 (1H, dd , J = 10.16, 2.33), 7.24 (1H, d, J = 2.33), 7.49 (2H, m), 7.58 (2H, m), 10.12 (1H, s), 10.45 (1H, s), 10.98 (1H, s). | MS (ESI+) m/z 369.17 (M + H)⁺. | |
| 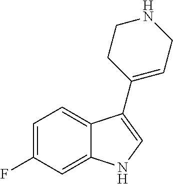 [SMILES: Fc1ccc2c(C3=CCNCC3)c[nH]c2c1] | 11011 | ¹H-NMR (600 MHz, DMSO-d6): 2.35 (2H, m), 2.93 (2H, m), 3.40 (2H, m), 6.15 (1H, m), 6.87 (1H, dt, J = 18.48, 2.46), 7.14 (1H, dd, J = 9.94, 2.46), 7.35 (1H, d, J = 2.13), 7.78 (1H, dd, J = 8.80, 5.47), 11.13 (1H, s) | Did not ionize | |
| 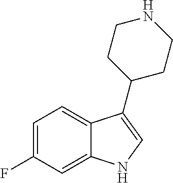 [SMILES: Fc1ccc2c(C3CCNCC3)c[nH]c2c1] | 11012 | ¹H-NMR (600 MHz, DMSO-d6): 1.53 (2H, m), 1.84 (2H, m), 2.63 (2H, m), 2.81 (2H, m), 3.00 (2H, m), 4.10 (1H, m), 6.80 (1H, m), 7.08 (2H, m), 7.53 (1H, dd, J = 8.83, 5.41), 10.82 (1H, s) | MS (ESI+) m/z 219.13 (M + H)⁺. | |
| 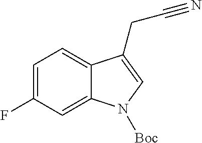 [SMILES: CC(C)(C)OC(=O)n1cc(CC#N)c2ccc(F)cc21] | 11013 | ¹H-NMR (600 MHz, CDCl₃): 1.67 (9H, s), 3.76 (2H, d, J = 1.19 Hz), 7.05 (1H, dt, J = 2.33, 13.29 Hz), 7.45 (1H, dd, J = 5.18, 8.61 Hz), 7.61 (1H, s), 7.90 (1H, s). | Did not ionize | |
| 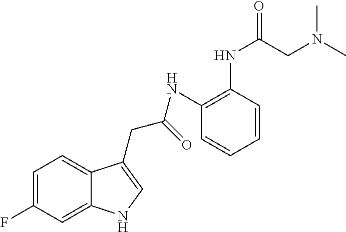 [SMILES: CN(C)CC(=O)Nc1ccccc1NC(=O)Cc1c[nH]c2cc(F)ccc12] | 11014 | ¹H-NMR (600 MHz, (CD₃)₂CO, Acetone): 3.05 (s, 6H), 3.86 (2H, s), 4.07 (2H, s), 6.85 (1H, m), 7.15 (3H, m), 7.38 (1H, s), 7.54 (1H, d, J = 9.08 Hz), 7.68 (2H, m), 9.28 (1H, s), 10.09 (1H, s), 10.44 (1H, s). | MS (ESI+) m/z 369.17 (M + H)⁺. | |
| 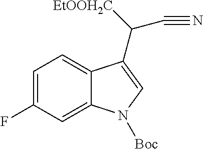 | 11015 | ¹H-NMR (600 MHz, CDCl₃) 1.30 (3H, t, J = 7.13 Hz), 1.68 (9H, s), 4.28 (2H, m, J = 5.51 Hz), 4.90 (1H, d, J = 0.68 Hz), 7.06 (1H, dt, J = 2.37, 8.89 Hz), 7.59 (1H, q, J = 4.64 Hz), 7.74 (1H, s), 7.90 (1H, s). | Did not ionize | |
| 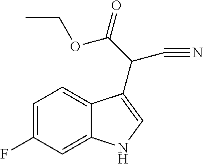 [SMILES: CCOC(=O)C(C#N)c1c[nH]c2cc(F)ccc12] | 11016 | ¹H-NMR (600 MHz, CDCl₃): 1.31 (t, 3H, J = 7.14), 4.28 (2H, m), 4.98 (1H, s), 6.99 (1H, d.t, J = 8/97, 2.33), 7.11 (dd, J = 9.36, 2.25), 7.37 (1H, d, J = 2.48), 7.67 (1H, dd, J = 8.78, 5.15), 8.31 (1H, s) | Did not ionize | |
| 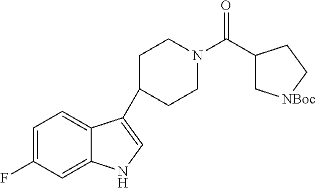 [SMILES: CC(C)(C)OC(=O)N1CCC(C(=O)N2CCC(c3c[nH]c4cc(F)ccc34)CC2)C1] | 11017 | ¹H-NMR (600 MHz, CDCl₃): 1.48 (9H, s), 1.65 (2H, m), 2.05 (1H, m), 2.06 (1H, m), 2.11 (1H, m), 2.17 (1H, m), 2.32 (1H, m), 2.78 (1H, m), 3.08 (1H, m), 3.27 (1H, m), 3.37 (1H, m), 3.50 (1H, m), 3.58 (1H, m), 3.73 (1H, m), 4.04 (1H, m), 4.78 (1H, m), 6.89 (1H, m), 6.94 (1H, m), 7.06 (1H, dd, J = 9.69, 2.21), 7.52 (1H, m), 8.36 (1H, s) | Did not ionize | |
| 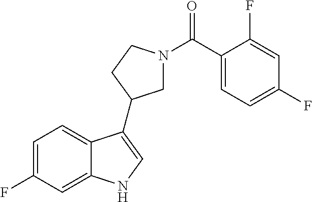 [SMILES: O=C(c1ccc(F)cc1F)N1CCC(c2c[nH]c3cc(F)ccc23)C1] | 11018 | mixture of two diastereomers ¹H- NMR (600 MHz, CDCl₃): 2.06-2.26 (m, 2H), 2.05-2.38 (2H, m), 3.35-3.54 (m, 2H), 3.93 (m, 1H), 4.15 (q, J = 7.14), 4.20 (m, 1H), 6.82-6.99 (m, 7H), 7.03-7.05 (m, 2H), 7.28 (1H, s), 7.43-7.51 (3H, m), 7.53 (dd, J = 7.54, 5.19), 8.35 (1H, s), 8.36 (1H, s). | MS (ESI+) m/z 345.12 (M + H)⁺. | |
| 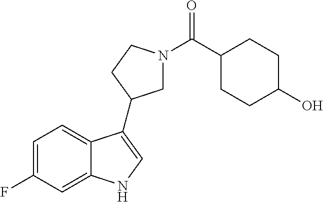 [SMILES: O=C(C1CCC(O)CC1)N1CCC(c2c[nH]c3cc(F)ccc23)C1] | 11019 | ¹H-NMR (600 MHz, CDCl₃): 1.29 (1H, m), 1.58 (3H, m), 1.86 (3H, m), 2.10 (1H, m), 2.32 (2H, m), 2.48 (1H, m), 3.61 (3H, m), 3.78 (1H, m), 4.00 (2H, m), 6.87 (1H, m), 6.98 (1H, m), 7.06 (1H, m), 7.48 (1H, m), 8.75 (1H, m). | MS (ESI+) m/z 331.18 (M + H)⁺. | |
| 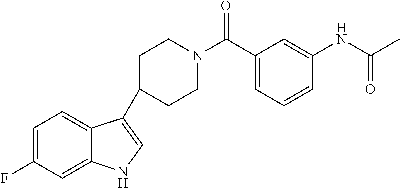 [SMILES: CC(=O)Nc1cccc(C(=O)N2CCC(c3c[nH]c4cc(F)ccc34)CC2)c1] | 11020 | ¹H-NMR (600 MHz, CDCl₃): 1.63 (1H, m), 1.74 (1H, m), 1.91 (1H, m), 2.00 (1H, m), 2.15 (3H, s), 2.97 (1H, m), 3.07 (1H, m), 3.19 (1H, m), 3.89 (1H, m), 4.82 (1H, m), 6.86 (1H, m), 6.88 (1H, m), 7.02 (1H, dd, J = 9.54, 2.28), 7.13 (1H, d, J = 7.50), 7.32 (1H, t, J = 7.87), 7.49 (1H, dd, J = 8.61, 5.15), 7.59 (1H, s), 7.61 (1H, d, J = 8.24), 8.41 (1H, s), 8.43 (1H, s) | MS (ESI+) m/z 380.18 (M + H)⁺. | |
| 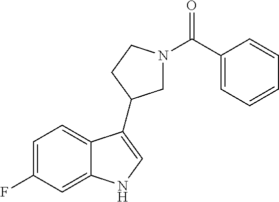 [SMILES: O=C(c1ccccc1)N1CCC(c2c[nH]c3cc(F)ccc23)C1] | 11021 | ¹H-NMR (600 MHz, water, D2O): 2.04-2.48 (5H, m), 3.50-3.85 (9H, m), 6.31 (1H, d.t, J = 9.32, 2.45), 7.094 (1H, s), 7.12 (1H, dd, J = 10.38, 2.25), 7.17 (1H, dd, J = 10.38, 2.25), 7.21 (1H, s), 7.39- 7.48 (12 H, m) | MS (ESI+) m/z 309.14 (M + H)⁺. | |
| 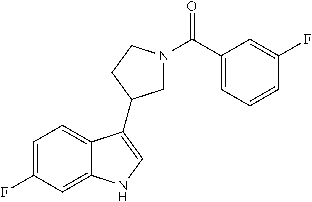 [SMILES: O=C(c1cccc(F)c1)N1CCC(c2c[nH]c3cc(F)ccc23)C1] | 11022 | ¹H-NMR (600 MHz, CDCl₃): 2.14 (1H, m), 2.42 (1H, m), 3.57 (2H, m), 3.76 (1H, m), 3.88 (1H, m), 4.17 (1H, m), 6.88 (1H, m), 6.99 (2H, m), 7.12 (1H, m), 7.26 (1H, m), 7.37 (2H, m), 7.52 (1H, m), 8.30 (1H, m) | MS (ESI+) m/z 327.13 (M + H)⁺. | |
| 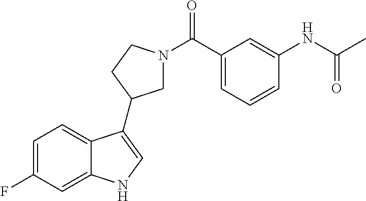 [SMILES: CC(=O)Nc1cccc(C(=O)N2CCC(c3c[nH]c4cc(F)ccc34)C2)c1] | 11023 | 1H-NMR (600 MHz, DMSO-d6): 1.26 (1H, m) 2.09 (3H, m), 2.35 (1H, m), 3.40- 3.94 (m, 4H), 4.03 (2H, m), 6.84 (1H, m), 7.11 (1H, m), 7.19 (1H, m), 7.24 (1H, s), 7.34 (1H, m), 7.52 (1H, m), 7.60 (1H, m), 7.82 (1H, d, J = 15.41), 10.05 (1H, d, J = 24), 10.98 (1H, d, J = 8.57) | MS (ESI+) m/z 366.16 (M + H)⁺. | |
| 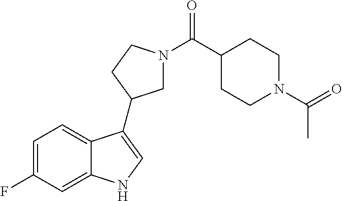 [SMILES: CC(=O)N1CCC(C(=O)N2CCC(c3c[nH]c4cc(F)ccc34)C2)CC1] | 11024 | mixture of two diastereomers ¹H-NMR (600 MHz, DMSO-d6): 1.35 (1H, m), 1.51 (m, 1H), 1.70 (m, 2H), 1.991 (3H, s), 1.994 (3H, s), 2.27 (1H, m), 2.38 (1H, m), 2.61 (1H, m), 2.72 (1H, m), 3.06 (2H, m), 3.59 (2H, m), 3.81 (3H, m), 4.07 (1H, m), 4.37 (2H, m), 6.85 (2H, m), 7.12 (2H, m), 7.18 (1H, d, J = 1.89), 7.23 (1H, s), 7.56 (IH, dd, J = 8.66, 5.32), 7.62 (1H, m), 10.95 (1H, s), 10.98 (1H, s) | MS (ESI+) m/z 358.20 (M + H)⁺. | |
| 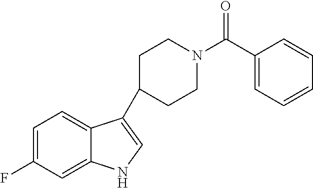 [SMILES: O=C(c1ccccc1)N1CCC(c2c[nH]c3cc(F)ccc23)CC1] | 11025 | mixture of two diastereomers ¹H-NMR (600 MHz, CDCl3): 1.64 (1H, m), 1.79 (1H, m), 2.98 (1H, m), 3.09 (1H, m), 3.21 (1H, m), 3.89 (1H, m), 4.89 (1H, m), 6.88 (1H, m), 6.94 (1H, d, J = 1.80), 7.04 (1H, dd, J = 9.56, 2.25), 7.38-7.48 (5H, m), 7.51 (1H, dd, J = 8.71, 5.20), 8.09 (1H, s) | MS (ESI+) m/z 323.16 (M + H)⁺. | |
| 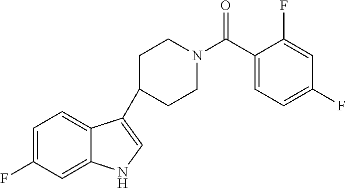 [SMILES: O=C(c1ccc(F)cc1F)N1CCC(c2c[nH]c3cc(F)ccc23)CC1] | 11026 | ¹H-NMR (600 MHz, DMSO, d6): 1.59 (1H, m), 1.91 (1H, m), 2.06 (1H, m), 2.36 (1H, m), 2.62 (1H, m), 2.96 (1H, m), 3.07 (1H, m), 3.26 (1H, m), 3.48 (1H, m), 4.62 (1H, m), 6.83 (1H, m), 7.11 (1H, m), 7.19 (1H, m), 7.38 (1H, m), 7.56 (1H, m), 10.96 (1H, s). | MS (ESI+) m/z 359.14 (M + H)⁺. | |
| 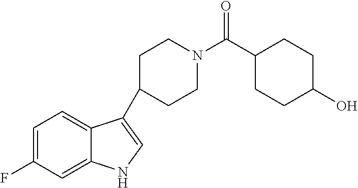 [SMILES: O=C(C1CCC(O)CC1)N1CCC(c2c[nH]c3cc(F)ccc23)CC1] | 11027 | ¹H-NMR (600 MHz, CDCl₃): 1.26- 1.40 (3H, m), 1.52-2.05 (10 H, m), 2.06-2.24 (4H, m), 2.56 (1H, m), 3.07 (1H, m), 6.89 (1H, dt, J = 9.20, 2.29), 6.95 (1H, s), 7.07 (1H, dd, J = 9.59, 2.29), 7.53 (1H, dd, J = 9.20, 5.26), 8.19 (1H, s). | MS (ESI+) m/z 345.20 (M + H)⁺. | |
| 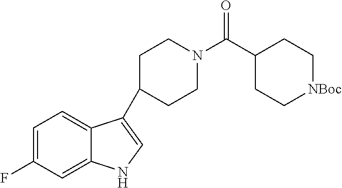 [SMILES: CC(C)(C)OC(=O)N1CCC(C(=O)N2CCC(c3c[nH]c4cc(F)ccc34)CC2)CC1] | 11028 | ¹H-NMR (600 MHz, CDCl₃): 1.29 (2H, m), 1.48 (9H, s), 1.64-1.82 (5H, m), 2.16 (1H, m), 2.38 (1H, m), 2.53 (1H, m), 2.77 (1H, m), 3.25 (1H, m), 3.45-3.89 (4H, m), 4.04 (1H, m), 4.20 (1H, m), 6.93 (1H, m), 7.02 (1H, m), 7.09 (1H, m), 7.53 (1H, m), 8.13 (1H, m). | Did not ionize | |
| 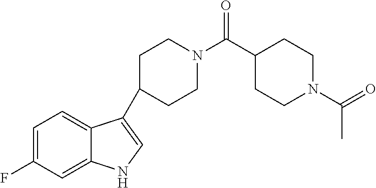 [SMILES: CC(=O)N1CCC(C(=O)N2CCC(c3c[nH]c4cc(F)ccc34)CC2)CC1] | 11029 | ¹H-NMR (600 MHz, CDCl₃): 1.23- 1.47 (3H, m), 1.57-1.97 (7H, m), 1.97-2.28 (6H, m) 2.62-2.88 (3H, m), 3.01-3.40 (2H, m), 6.90 (1H, dt, J = 9.29, 2.25), 6.95 (1H, d, J = 1.79), 7.07 (1H, dd, J = 9.56, 2.25), 7.53 (1H, dd, J = 9.29, 5.22), 8.16 (1H, s) | MS (ESI+) m/z 372.21 (M + H)⁺. | |
| 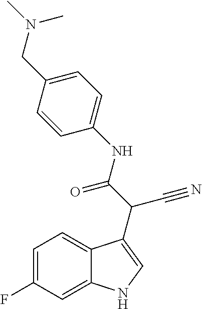 [SMILES: CN(C)Cc1ccc(NC(=O)C(C#N)c2c[nH]c3cc(F)ccc23)cc1] | 11030 | ¹H-NMR (600 MHz, DMSO) 2.69 (6H, s), 4.20 (2H, s), 5.52 (1H, s), 6.53 (1H, s), 6.97 (1H, m, J = 4.17 Hz), 7.22 (1H, dd, J = 2.38, 9.88 Hz), 7.42 (2H, d, J = 8.58 Hz), 7.50 (1H, d, J = 2.54 Hz), 7.63 (2H, d, J = 8.57 Hz), 7.73 (1H, dd, J = 5.45, 8.81 Hz), 10.56 (1H, s), 11.40 (1H, s). | MS (ESI+) m/z 351.16 (M + H)⁺. | |
| 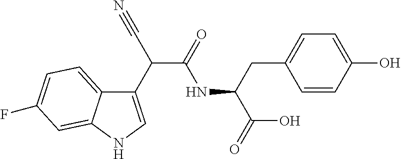 [SMILES: N#CC(C(=O)N[C@@H](Cc1ccc(O)cc1)C(=O)O)c1c[nH]c2cc(F)ccc12] | 11031 | | MS (ESI+) m/z 382.12 (M + H)⁺. | |
| 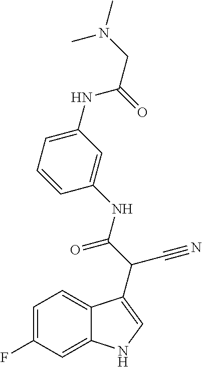 [SMILES: CN(C)CC(=O)Nc1cccc(NC(=O)C(C#N)c2c[nH]c3cc(F)ccc23)c1] | 11032 | ¹H-NMR (600 MHz, DMSO-d6): 2.86 (6H, s), 4.09 (2H, s), 5.48 (1H, s), 6.96 (1H, dt, J = 2.26, 9.30 Hz), 7.18 (1H, d, J = 7.80 Hz), 7.21 (1H, dd, J = 2.25, 9.93 Hz), 7.29 (1H, t, J = 8.07 Hz), 7.33 (1H, d, J = 8.34 Hz), 7.48 (1H, d, J = 2.46 Hz), 7.73 (1H, q, J = 4.70 Hz), 7.99 (1H, s), 9.70 (1H, s), 10.47 (1H, s), 10.54 (1H, s), 11.38 (1H, s). | MS (ESI+) m/z 394.17 (M + H)⁺. | |
| 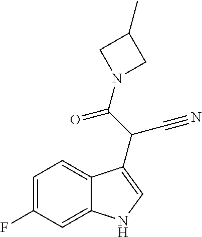 | 11033 | **Spectrum split due to rotamers ¹H-NMR (600 MHz, DMSO-d6) 1.00 (1.2H, d, J = 6.86 Hz), 1.15 (1.5H, d, J = 6.88 Hz), 2.53 (0.6H, q, J = 1.92 Hz), 2.66 (0.48H, q, J = 7.34 Hz), 3.38 (1H, q, J = 3.14 Hz), 3.48 (0.6H, q, J = 5.24 Hz), 3.85 (1H, d, J = 6.90 Hz), 3.89 (0.5H, t, J = 9.06 Hz), 4.03 (0.5H, t, J = 8.94 Hz), 4.40 (0.4H, t, J = 8.40 Hz), 5.54 (1H, d, J = 7.68 Hz), 6.95 (1H, m, J = 2.81 Hz), 7.20 (1H, m, J = 3.12 Hz), 7.44 (1H, d, J = 2.52 Hz), 7.62 (1H, q, J = 4.70 Hz), 11.36 (1H, s). | MS (ESI+) m/z 345.20 (M + H)⁺. 372.12 | |
| 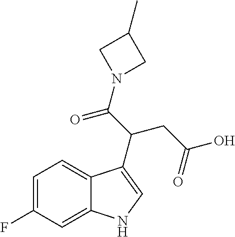 [SMILES: CC1CN(C(=O)C(CC(=O)O)c2c[nH]c3cc(F)ccc23)C1] | 11034 | ¹H-NMR (600 MHZ, DMSO-d6) 1.07 (3H, q, J = 34.38 Hz), 2.46 (1H, t, J = 5.20 Hz), 3.04 (1H, m, J = 7.57 Hz), 3.29 (2H, q, J = 5.13 Hz), 3.39 and 4.38 (1H, q, J = 5.02 Hz), 3.84 (1H, m, J = 5.73 Hz), 3.95 (1H, q, J = 6.31 Hz), 4.09 (1H, q, J = 5.02 Hz), 6.84 (1H, m, J = 3.35 Hz), 7.11 (1H, m, J = 2.57 Hz), 7.20 (1H, d, J = 2.35 Hz), 7.58 (1H, m, J = 3.59 Hz), 11.03 (1H, s), 12.12 (1H, d, J = 3.68 Hz). | MS (ESI+) m/z 305.13 (M + H)⁺. | |
| 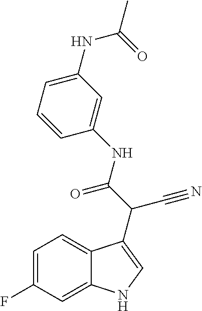 [SMILES: CC(=O)Nc1cccc(NC(=O)C(C#N)c2c[nH]c3cc(F)ccc23)c1] | 11035 | ¹H-NMR (600 MHz, DMSO-d6) 2.02 (3H, s), 5.47 (1H, s), 6.97 (1H, m, J = 4.19 Hz), 7.22 (3H, m, J = 4.23 Hz), 7.26 (1H, td, J = 1.74, 7.60 Hz), 7.47 (1H, d, J = 2.54 Hz), 7.73 (1H, q, J = 4.73 Hz), 7.89 (1H, s), 9.95 (1H, s), 10.40 (1H, s), 11.37 (1H, s). | MS (ESI+) m/z 351.13 (M + H)⁺. | |
| 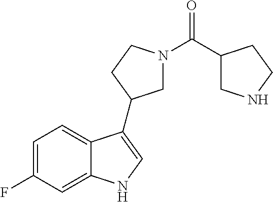 [SMILES: O=C(C1CCNC1)N1CCC(c2c[nH]c3cc(F)ccc23)C1] | 11036 | mixture of two diastereomers ¹H- NMR (600 MHz, DMSO-d6): 1.29 (2H, m), 1.87-2.03 (3H, m), 2.16- 2.32 (3H, m), 2.39 (1H, m), 3.21 (3H, m), 3.36 (3H, m), 3.44-3.68 (9H, m), 3.75 (2H, m), 3.90 (1H, m), 6.86 (2H, m), 7.13 (2H, m), 7.21 (1H, m), 7.24 (1H, m), 7.56 (1H, dd, J = 8.80, 5.63), 7.62 (1H, dd, J = 8.80, 5.63), 8.88 (2H, s), 11.00 (1H, s). | MS (ESI+) m/z 302.17 (M + H)⁺. | |
| 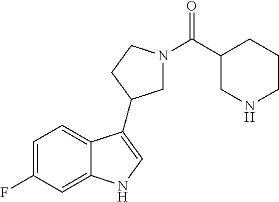 [SMILES: O=C(C1CCCNC1)N1CCC(c2c[nH]c3cc(F)ccc23)C1] | 11037 | mixture of two diastereomers ¹H- NMR (600 MHz, DMSO-d6): 1.61-2.08 (7H, m), 2.09-3.06 (7H, m), 3.11-3.36 (6H, m), 3.44-3.72 (10H, m), 3.38 (2H, m), 6.86 (2H, m), 7.13 (2H, m), 7.21 (1H, m), 7.24 (1H, m), 7.56 (1H, m), 7.62 (1H, m), 8.55 (2H, s). | MS (ESI+) m/z 316.18 (M + H)⁺. | |
| 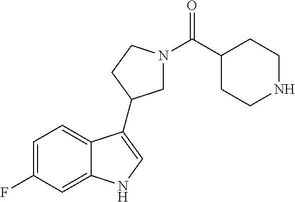 [SMILES: O=C(C1CCNCC1)N1CCC(c2c[nH]c3cc(F)ccc23)C1] | 11038 | mixture of two diastereomers 1H- NMR (600 MHz, DMSO-d6): 1.68- 1.79 (3H, m), 1.80-1.90 (3H, m), 2.00 (1H, m), 2.07 (1H, m), 2.27 (1H, m), 2.38 (1H, m), 2.79 (2H, m), 2.92 (4H, m), 3.26-3.35 (8H, m), 3.44 (2H, m), 3.54 (1H, m), 3.62 (2H, m), 3.74 (1H, m), 3.84 (1H, m), 4.06 (1H, m), 6.85 (2H, m), 7.13 (2H, m), 7.19 (1H, d, J = 1.80), 7.23 (1H, d, J = 1.80), 7.56 (1H, dd, J = 8.42, 5.41), 7.62 (1H, dd, J = 8.42, 5.41), 8.29 (s, 1H), 8.57 (1H, s), 10.98 (1H, s), 11.01 (1H, s) | MS (ESI+) m/z 316.18 (M + H)⁺. | |
| 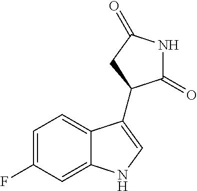 [SMILES: O=C1C[C@H](c2c[nH]c3cc(F)ccc23)C(=O)N1] | 11039 | ¹H-NMR (600 MHz, DMSO-d6): 2.77 (1 H, dd, J = 18.71, 5.40 Hz), 3.17 (1H, dd, J = 18.71, 9.31 Hz), 4.34 (1H, dd, J = 5.40, 9.31 Hz), 6.87 (1H, m), 7.15 (1H, dd, J = 10.13, 2.48), 7.33, (1H, d, J = 2.48), 7.43 (1H, dd, J = 5.27, 8.69), 11.29 (1H, s), 11.10 (1H, s). | MS (ESI+) m/z 233.07 (M + H)⁺. | |
| 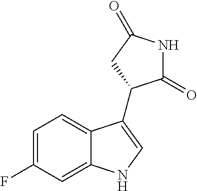 [SMILES: O=C1C[C@@H](c2c[nH]c3cc(F)ccc23)C(=O)N1] | NMX0- 11040 | ¹H-NMR (600 MHz, DMSO-d6): 2.77 (1 H, dd, J = 18.71, 5.40 Hz), 3.17 (1H, dd, J = 18.71, 9.31 Hz), 4.34 (1H, dd, J = 5.40, 9.31 Hz), 6.87 (1H, m), 7.15 (1H, dd, J = 10.13, 2.48), 7.33, (1H, d, J = 2.48), 7.43 (1H, dd, J = 5.27, 8.69), 11.29 (1H, s), 11.10 (1H, s). | MS (ESI+) m/z 233.07 (M + H)⁺. | |
| 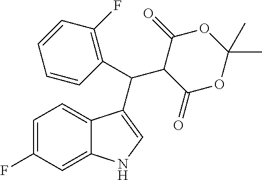 [SMILES: CC1(C)OC(=O)C(C(c2ccccc2F)c2c[nH]c3cc(F)ccc23)C(=O)O1] | 11041 | ¹H-NMR (600 MHz, CDCl₃): 1.63 (3H, s), 1.76 (3H, s), 4.34 (1H, dd, J = 3.25, 1.98), 5.78 (1H, d, J = 3.25), 6.84 (1H, m), 7.03 (2H, m), 7.07 (1H, m), 7.23 (2H, m), 7.28 (1H, m), 7.38 (1H, dd, J = 8.60, 5.18), 8.18 (1H, s) | Did not ionize | |
| 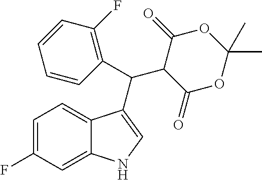 [SMILES: CC1(C)OC(=O)C(C(c2ccccc2F)c2c[nH]c3cc(F)ccc23)C(=O)O1] | 11042 | ¹H-NMR (600 MHz, CDCl₃): 1.63 (3H, s), 1.76 (3H, s), 4.34 (1H, dd, J = 3.25, 1.98), 5.78 (1H, d, J = 3.25), 6.84 (1H, m), 7.03 (2H, m), 7.07 (1H, m), 7.23 (2H, m), 7.28 (1H, m), 7.38 (1H, dd, J = 8.60, 5.18), 8.18 (1H, s) | Did not ionize | |
| 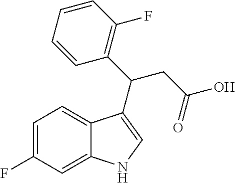 [SMILES: O=C(O)CC(c1ccccc1F)c1c[nH]c2cc(F)ccc12] | 11043 | ¹H-NMR (600 MHz, CDCl₃): 3.14 (2H, m), 5.08 (1H, t, J = 8.16), 6.82 (1H, m), 7.02 (2H, m), 7.05 (1H, m), 7.09 (1H, m), 7.18 (1H, m), 7.35 (1H, dd,., J = 8.72, 5.27), 8.02 (1H, s) | MS (ESI+) m/z 202.10 (M + H)⁺. | |
| 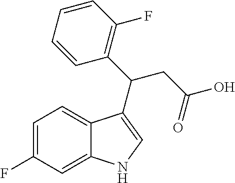 [SMILES: O=C(O)CC(c1ccccc1F)c1c[nH]c2cc(F)ccc12] | 11044 | ¹H-NMR (600 MHz, CDCl₃): 3.14 (2H, m), 5.08 (1H, t, J = 8.16), 6.82 (1H, m), 7.02 (2H, m), 7.05 (1H, m), 7.09 (1H, m), 7.18 (1H, m), 7.35 (1H, dd, J = 8.72, 5.27), 8.02 (1H, s) | MS (ESI+) m/z 202.10 (M + H)⁺. | |
| 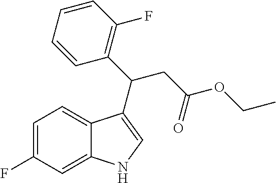 [SMILES: CCOC(=O)CC(c1ccccc1F)c1c[nH]c2cc(F)ccc12] | 11045 | ¹H-NMR (600 MHz, CDCl₃): 1.14 (3H, t, J = 7.24), 3.12 (2H, m), 4.07 (2H, m), 5.10 (1H, t, J = 7.63), 7.68 (1H, m), 7.04 (2H, m), 7.10 (1H, m), 7.19 (1H, m), 7.27 (1H, m), 7.39 (1H, dd, J = 8.70, 5.30), 8.07 (1H, s) | MS (ESI+) m/z 330.13 (M + H)⁺. | |
| 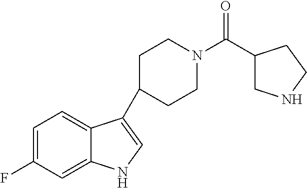 [SMILES: O=C(C1CCNC1)N1CCC(c2c[nH]c3cc(F)ccc23)CC1] | 11046 | mixture of two isomers ¹H-NMR (600 MHz, DMSO-d6): 1.53 (1H, m), 1.78 (1H, m), 1.99 (1H, m), 2.22 (1H, m), 2.76 (1H, m), 2.96 (2H, m), 3.06 (1H, m), 3.21 (2H, m), 3.56 (2H, m), 3.86 (2H, m), 4.04 (1H, m), 4.50 (1H, m), 6.85 (1H, m), 7.11 (2H, m), 7.56 (1H, m), 10.91 (1H, s). | MS (ESI+) m/z 316.18 (M + H)⁺. | |
| 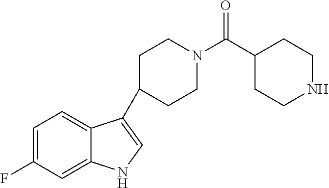 [SMILES: O=C(C1CCNCC1)N1CCC(c2c[nH]c3cc(F)ccc23)CC1] | 11047 | ¹H-NMR (600 MHz, DMSO-d6) 1.45 (1H, m), 1.54 (1H, m), 1.69-1.84 (3H, m), 1.99 (2H, m), 2.70 (1H, t, J = 12.38), 2.90-3.11 (4H, m), 3.21 (1H, t, J = 12.83), 3.29 (3H, m), 4.08 (1H, d, J = 12.62), 4.51 (1H, d, J = 12.62), 6.83 (1H, J = 9.46), 7.11 (2H, m), 7.56 (1H, m), 10.90 (1H, s) | MS (ESI+) m/z 330.20 (M + H)⁺. | |
| 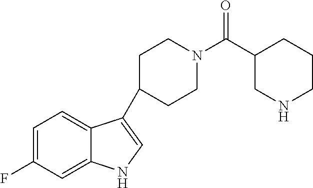 [SMILES: O=C(C1CCCNC1)N1CCC(c2c[nH]c3cc(F)ccc23)CC1] | 11048 | ¹H-NMR (600 MHz, DMSO-d6): 1.39-2.10 (8H, m), 2.75 (1 H, m), 2.90-3.32 (7H, m), 3.95 (1H, d, J = 12.92), 4.52 (1H, d, J = 12.92), 6.83 (1H, m), 7.11 (2H, d, J = 10.83), 7.56 (1H, m), 10.92 (1H, s) | MS (ESI+) m/z 330.20 (M + H)⁺. | |
| 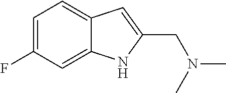 | 11049 | ¹H-NMR (600 MHz, DMSO-d6): 2.18 (6H, s), 3.51 (2H, s), 6.26 (1H, s), 6.79 (1H, m), 7.04 (1H, dd, J = 10.10, 2.08), 7.42 (1H, dd, 8.40, 5.42), 11.08 (1H, s). | | |
| 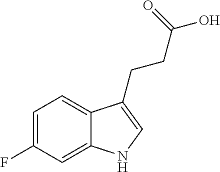 [SMILES: O=C(O)CCc1c[nH]c2cc(F)ccc12] | 11050 | ¹H-NMR (600 MHz, DMSO-d6): 2.56 (2H, t, J = 7.35), 2.90 (2H, t, J = 7.35), 6.82 (1H, m), 7.09 (2H, m), 7.50 (1H, dd, J = 8.62, 5.49), 10.57 (1H, s), 12.10 (1H,s). | | |
| 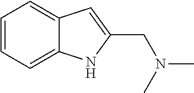 | 11051 | ¹H-NMR (600 MHz, DMSO-d6): 2.22 (6H, s), 3.58 (2H, s), 6.27 (1H, s), 6.94 (1H, t, J = 7.36), 7.02 (1H, t, J = 7.77), 7.31 (1H, d, J = 7.77), 7.44 (1H, d, J = 7.92), 11.00 (1H, s). | | |
| 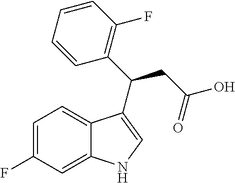 [SMILES: O=C(O)C[C@H](c1ccccc1F)c1c[nH]c2cc(F)ccc12] | 11052 | ¹H-NMR (600 MHz, CDCl₃): 3.14 (2H, m), 5.08 (1H, t, J = 8.16), 6.82 (1H, m), 7.02 (2H, m), 7.05 (1H, m), 7.09 (1H, m), 7.18 (1H, m), 7.35 (1H, dd, J = 8.72, 5.27), 8.02 (1H, s) | MS (ESI+) m/z 202.10 (M + H)⁺. | |
| 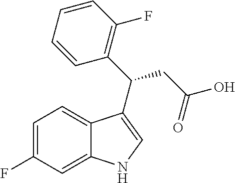 [SMILES: O=C(O)C[C@@H](c1ccccc1F)c1c[nH]c2cc(F)ccc12] | 11053 | ¹H-NMR (600 MHz, CDCl₃): 3.14 (2H, m), 5.08 (1H, t, J = 8.16), 6.82 (1H, m), 7.02 (2H, m), 7.05 (1H, m), 7.09 (1H, m), 7.18 (1H, m), 7.35 (1H, dd, J = 8.72, 5.27), 8.02 (1H, s) | MS (ESI+) m/z 202.10 (M + H)⁺. | |
| 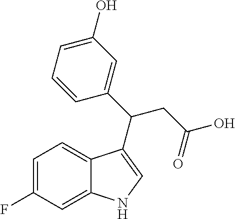 [SMILES: O=C(O)CC(c1cccc(O)c1)c1c[nH]c2cc(F)ccc12] | 11054 | ¹H-NMR (600 MHz, DMSO, d6): 2.00 (1H, m), 2.18 (1H, m), 5.70 (1H, m), 5.56 (1H, m), 6.70-6.78 (2H, m), 6.80 (1H, m), 7.05 (1H, m), 7.11 (1H, m), 7.22 (1H, m), 7.38 (1H, m), 9.15 (1H, s), 10.87 (1H, s). | | |
| 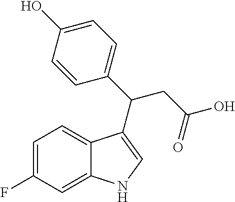 [SMILES: O=C(O)CC(c1ccc(O)cc1)c1c[nH]c2cc(F)ccc12] | 11055 | ¹H-NMR (600 MHz, DMSO, d6): 2.09 (1H, m), 2.44 (1H, m), 2.57 (1H, m), 6.28 (1H, m), 6.64 (1H, m), 6.80 (1H, m), 7.03 (1H, m), 7.41 (1H, m), 7.48 (1H, m), 7.79 (1H, m), 8.58 (1H, m), 10.12 (1H, s). | | |
| 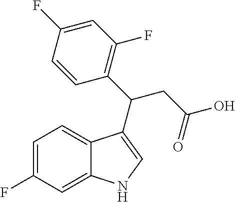 [SMILES: O=C(O)CC(c1ccc(F)cc1F)c1c[nH]c2cc(F)ccc12] | 11056 | ¹H-NMR (600 MHz, DMSO, d6): 3.04 (2H, m), 4.85 (1H, t, J = 7.96), 6.79 (1H, m), 6.97 (1H, d.t, J = 8.45, 2.51), 7.10 (1H, dd, J = 10.25, 2.28), 7.16 (1H, m), 7.28 (1H, d, J = 2.28), 7.32 (1H, dd, J = 8.81, 5.36), 7.39 (1H, m), 11.02 (1H, s), 12.28 (1H, s). | | |
| 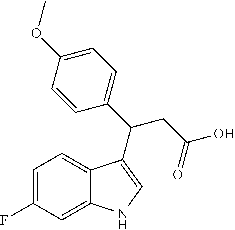 [SMILES: COc1ccc(C(CC(=O)O)c2c[nH]c3cc(F)ccc23)cc1] | 11057 | ¹H-NMR (600 MHz, DMSO, d6): 2.94 (2H, m), 3.68 (3H, m), 4.54 (1H, t, J = 7.82), 6.74 (1H, m), 6.79 (1H, m), 7.07 (1H, dd, J = 10.11, 2.35), 7.23 (2H, m), 7.28 (2H, m), 10.93 (1H, s), 12.06 (1H, s) | | |
| 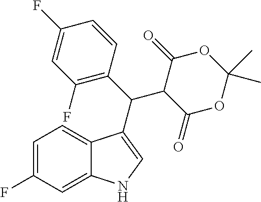 [SMILES: CC1(C)OC(=O)C(C(c2ccc(F)cc2F)c2c[nH]c3cc(F)ccc23)C(=O)O1] | 11058 | ¹H-NMR (600 MHz, acetone): 1.67 (3H, s), 1.89 (3H, s), 4.80(1H, d, 3.41), 5.78 (1H, d, J = 341), 6.81 (1H, m), 6.86 (1H, m), 7.02 (1H, m), 7.17 (1H, dd, J = 9.91. 2.31), 7.32- 7.40 (3H, m), 10.36 (1H, s) | | |
| 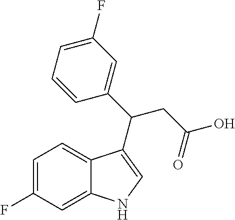 [SMILES: O=C(O)CC(c1cccc(F)c1)c1c[nH]c2cc(F)ccc12] | 11059 | ¹H-NMR (600 MHz, DMSO, d6): 2.92 (1H, m), 3.04 (1H, m), 4.63 (1H, t, J = 7.78), 6.76 (1H, m), 6.95 (1H, dt, J = 8.46, 2.40), 7.08 (1H, dd, J = 10.08, 2.40), 7.16 (1H, m), 7.19 ( 1H, m), 7.27 ( 1H, m), 7.33 (1H, d, J = 2.29), 7.36 (1H, dd, J = 8.81, 5.52), 11.00 (1H, s) | | |
| 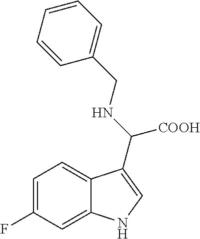 [SMILES: O=C(O)C(NCc1ccccc1)c1c[nH]c2cc(F)ccc12] | 11060 | ¹H-NMR (600 MHz, DMSO, d6): 3.34 (1H, s), 3.87 (2H, m), 4.43 (1H, s), 6.85 (1H, m), 7.14 (1H, dd, J = 10.05, 2.34), 7.30-7.40 (4H, m), 7.60 (1H, dd, J = 8.72, 5.54), 11.15 (1H, s). | | |
| 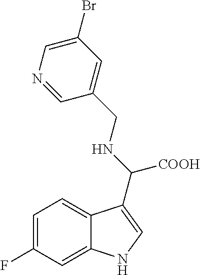 [SMILES: O=C(O)C(NCc1cncc(Br)c1)c1c[nH]c2cc(F)ccc12] | 11061 | ¹H-NMR (600 MHz, DMSO, d6): 3.32 (1H, s), 3.80 (2H, m), 4.50 (1H, s), 6.86 (1H, t, J = 9.99), 7.13 (1H, d, J = 9.99) 7.33 (1H, s), 7.64 (1H, m), 8.02 (1H, s), 8.47 (1H, s), 8.56 (1H, s), 11.19 (1H,s). | | |
| 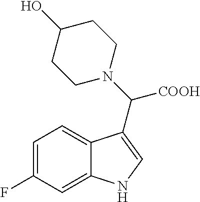 [SMILES: O=C(O)C(c1c[nH]c2cc(F)ccc12)N1CCC(O)CC1] | 11062 | ¹H-NMR (600 MHz, DMSO, d6): 1.56 (2H, m), 1.82 (2H, m), 2.73 (2H, m), 2.99 (2H, m), 3.61 (1H, m), 4.49(1H, s), 6.88 (1H, d.t, J = 9.31, 2.30), 7.11 (1H, dd, J = 9.99, 2.30), 7.40 (1H, d, J = 2.46), 7.75 (1H, dd, J = 8.76, 5.54), 11.30 (1H, s). | | |
| 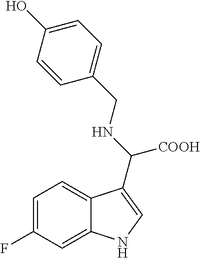 [SMILES: O=C(O)C(NCc1ccc(O)cc1)c1c[nH]c2cc(F)ccc12] | 11063 | ¹H-NMR (600 MHz, DMSO, d6): 3.17 (2H, m), 3.82 (1H, m), 6.74 (2H, m), 6.85 (1H, m), 7.14 (1H, m), 7.20 (2H, m), 7.31 (1H, m), 7.58 (1H, m), 9.58 (1H, m), 11.18(1H, s). | | |
| 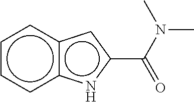 | 11064 | ¹H-NMR (600 MHz, D₂O): 3.06 (s, 3H), 3.32 (s, 3H), 6.97 (s, 1H), 7.12 (1H, t, J = 16.01), 7.28 (1H, t, J = 16.01), 7.48 (1H, t, J = 16.01), 7.48 (1H, d, J = 8.35), 7.68 (1H, d, J = 8.35). | MS (ESI+) m/z 188.1 (M)⁺. | |
| 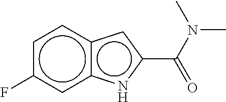 | 11065 | ¹H-NMR (600 MHz, DMSO, d₆): 2.87 (6H, s), 6.91 (2H, m), 7.14 (1H, d.d., J = 9.58, 2.21), 7.63 (1H, d.d., J = 9.58, 5.63), 11.59 (1H, s). | MS (ESI+) m/z 206.1 (M)⁺. | |
| 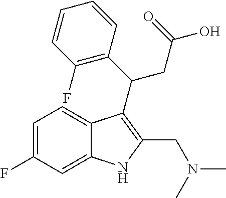 [SMILES: CN(C)Cc1[nH]c2cc(F)ccc2c1C(CC(=O)O)c1ccccc1F] | 11066 | ¹H-NMR (600 MHz, D₂O): 2.31 (m, 2H), 2.43 (m, 2H), 2.59 (s, 6H), 3.63 (m, 1H), 7.00 (1H, m), 7.06- 7.17 (3H, m), 7.24 (1H, m), 7.33 (1H, m), 7.61 (1H, m). | Did not ionize | |
| Compound | | | | LC-MS |
| No. | Structure | Purity | ¹H NMR | (M + H)⁺ |
| 11067 | 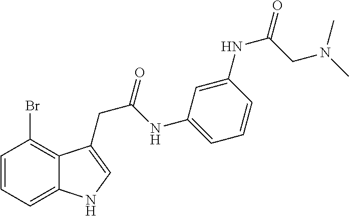 [SMILES: CN(C)CC(=O)Nc1cccc(NC(=O)Cc2c[nH]c3cccc(Br)c23)c1] | 99% | ¹H NMR (600 MHz, DMSO-d₆) δ 11.29 (s, 1H), 10.52 (s, 1H), 10.05 (s, 1H), 8.00-7.98 (m, 1H), 7.49-7.39 (d, 1H), 7.36-7.33 (m, 2H), 7.28- 7.27 (d, 2H), 7.31- 7.25 (d, 1H), 6.99-6.87 (dd, 1H), 4.09 (s, 2H), 3.99 (s, 2H), 2.86 (s, 6H). | 428.1 |
| 11068 | 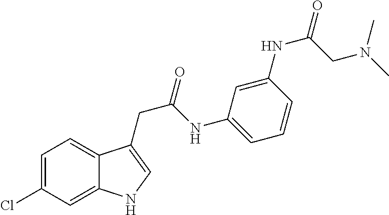 [SMILES: CN(C)CC(=O)Nc1cccc(NC(=O)Cc2c[nH]c3cc(Cl)ccc23)c1] | 99% | ¹H NMR (600 MHz, DMSO-d₆) δ 11.06 (s, 1H), 10.11 (s, 1H), 9.67 (s, 1H), 7.99-7.96 (m, 1H), 7.62-7.57 (d, 1H), 7.40-7.38 (d, 1H), 7.32- 7.30 (m, 1H), 7.30- 7.28 (d, 1H), 7.27-7.25 (m, 1H), 7.20-7.18 (dd, 1H), 7.02-6.99(dd, 1H), 3.71 (s, 2H), 3.05 (s, 2H), 2.08(s, 6H). | 384.1 |
| 11070 | 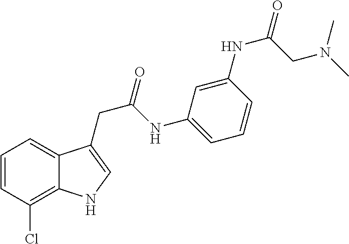 [SMILES: CN(C)CC(=O)Nc1cccc(NC(=O)Cc2c[nH]c3c(Cl)cccc23)c1] | 99% | ¹H NMR (600 MHz, DMSO-d₆) δ 11.31 (s, 1H), 10.52 (s, 1H), 10.20 (s, 1H), 9.73 (s, 1H), 8.02-7.99 (s, 1H), 7.60- 7.59 (d, 1H), 7.34-7.32 (m, 2H), 7.28-7.24 (m, 2H), 7.18-7.16 (d, 1H), 7.02-6.99 (dd, 1H), 4.10 (s, 2H), 3.75 (s, 2H), 2.87(s, 6H). | 384.1 |
| 11071 | 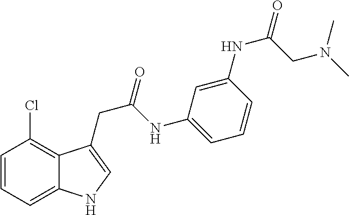 [SMILES: CN(C)CC(=O)Nc1cccc(NC(=O)Cc2c[nH]c3cccc(Cl)c23)c1] | 98% | ¹H NMR (600 MHz, DMSO-d₆) δ 11.27 (s, 1H), 10.51 (s, 1H), 10.05 (s, 1H), 9.71 (s, 1H), 7.99-7.98 (s, 1H), 7.62- 7.32 (m, 2H), 7.27-7.24 (m, 2H), 7.13-7.04 (dd, 1H), 6.98-6.96(d, 1H), 4.10 (s, 2H), 3.98 (s, 2H), 2.87(s, 6H). | 384.1 |
| 11072 | 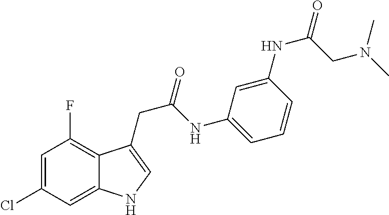 [SMILES: CN(C)CC(=O)Nc1cccc(NC(=O)Cc2c[nH]c3cc(Cl)cc(F)c23)c1] | 97% | ¹H NMR (600 MHz, DMSO-d₆) δ 11.33 (s, 1H), 10.05 (s, 1H), 9.68 (s, 1H), 7.97-7.96 (m, 1H), 7.34-7.32 (m, 1H), 7.31-7.30 (m, 1H), 7.28-7.26 (m, 1H), 7.26- 7.25 (m, 1H), 7.21-7.18 (m, 1H), 6.85-6.84(m, 1H), 3.82 (s, 2H), 3.06 (s, 2H), 2.28(s, 6H). | 402.1 |
| 11073 | 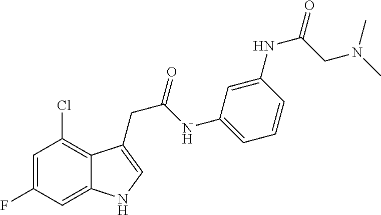 [SMILES: CN(C)CC(=O)Nc1cccc(NC(=O)Cc2c[nH]c3cc(F)cc(Cl)c23)c1] | 98% | ¹H NMR (600 MHz, DMSO-d₆) δ 11.34 (s, 1H), 10.52 (s, 1H), 10.07 (s, 1H), 9.74 (s, 1H), 7.99-7.98 (m, 1H), 7.31- 7.26 (m, 2H), 7.18-7.17 (dd, 2H), 6.97-6.95 (dd, 1H), 4.10 (s, 2H), 3.95 (s, 2H), 2.87(s, 6H). | 402.1 |

### Characterization of the Compounds

### Example 80—Materials and Methods

### Cloning, Expression and Purification of Proteins.

The codon optimized sequences for HRas^{G12V} (aa 1-166) and human SOS1 (SOS^{cat}, aa 564-1049) were synthesized and cloned into the pET-28a(+) plasmid at GenScript (https://www.genscript.com). Proteins were expressed in E. coli BL21 (DE3) cells in Terrific Broth (TB) media and induced with 0.5 mM IPTG at 25° C. overnight. Cells were harvested by centrifugation and cell pellets were processed by sonication in 50 mM Tris, pH 8, 500 mM NaCl, 5 mM Tris(2-carboxyethyl)phosphine hydrochloride (TCEP). Cell lysates were centrifuged at 20,000 g for 30 minutes at 4° C. and supernatant was diluted in TCEP-free lysis buffer to reduce TCEP concentration to 1 mM. The diluted solution was loaded onto a HisTrap HP column (GE Healthcare) in a buffer containing 50 mM Tris, pH 8, 500 mM NaCl, 1 mM TCEP and the bound proteins were eluted with 300 mM imidazole in the same buffer. For HRAS NMR studies, HRAS N-terminal His-tag was cleaved by incubation with tobacco etch virus protease (TEV) overnight at 4° C. in a SnakeSkin Dialysis Tubing (Thermo Scientific) to remove excess imidazole. TEV was removed using a nickel column and the proteins were further purified on a size-exclusion (SEC) Superdex 75 column (GE Healthcare) in buffers containing 25 mM sodium phosphate, pH 7.4, 150 mM NaCl, 5 mM MgCl₂, 1 mM TCEP and 25 mM Tris, pH 7.4, 50 mM NaCl, 1 mM dithiothreitol (DTT) for HRAS and SOS, respectively. Fractions containing the respective proteins were pooled, concentrated using Amicon centrifugal filters (Millipore), flash frozen in liquid nitrogen and stored at −80° C. The purity of HRAS and SOS was greater than 95% by SDS-PAGE.

Uniformly ¹⁵N-labeled HRAS was purified using the same steps as described above but was expressed in M9 minimal media with ¹⁵NH₄Cl as the sole nitrogen source.

### Nucleotide Loading.

For biophysical assessments, HRAS was loaded with GDP before the size-exclusion chromatography step by incubation with 20 mM EDTA and 5 mM GDP at room temperature for 30 minutes. The solution was then buffer-exchanged in EDTA- and nucleotide-free buffer with 5 mM MgCl₂ before loading into the SEC column.

For the nucleotide release assay, HRAS was buffer exchanged in MgCl₂-free buffer and incubated with 20 mM EDTA and 2 mM BODIPY FL GDP (Invitrogen) for 1.5 h at room temperature. The reaction was then supplemented with 10 mM MgCl₂ and incubated for another 30 minutes at room temperature. EDTA and the excess nucleotides were removed by buffer exchange into 25 mM Tris, pH 7.4, 50 mM NaCl, 1 mM DTT, 10 mM MgCl₂.

### Nmr Experiments.

All NMR experiments were collected using 3 mm NMR tubes on a 600 MHz Bruker Avance III spectrometer equipped with a QCI helium cryoprobe and a SampleJet sample changer. The buffer used for NMR experiments was 25 mM sodium phosphate pH 7.4, 150 mM NaCl, 5 mM MgCl₂, 1 mM TCEP-_{d16}, 10% D₂O. Data were processed using Bruker TopSpin.

### Measurement of Dissociation Constants (K) by NMR.

¹H/¹⁵N Fast-HSQC experiments (Mori et al., Journal of Magnetic Resonance, Series B, Volume 108, Issue 1, July 1995, Pages 94-98) were recorded at a protein concentration of 50-100 μM.

Dissociation constants were obtained by monitoring changes in chemical shifts as a function of ligand concentration. The changes in chemical shifts (d) were calculated according to the following equation (Williamson, Progress in Nuclear Magnetic Resonance Spectroscopy, Volume 73, August 2013, Pages 1-16): d=√(1/2[δ_H\hat{ }2+(α−δ_N\hat{ }2)]), Where δ are the changes in chemical shift in ppm for ¹H and ¹⁵N and the correction factor (α) was set at 0.15.

¹H 1D protein-observed experiments were recorded with 15-50 μM protein. The standard Bruker 1D ¹H sequence with excitation sculpting (zgesgp) was employed. A relaxation delay of 1 s was employed in order to use 256 scans while still keeping experimental time fairly short. Changes in protein chemical shift or peak intensity in the methyl region were monitored against compound concentration.

### Measurement of Dissociation Constants (K) by Microscale Thermophoresis (MST).

MST experiments were performed with a Monolith NT.115 Pico (NanoTemper Technologies, Munich, Germany). Fluorescence labeling of GDP-HRas^{G12V} was achieved according to the manufacturer's protocol of the His-Tag Labeling Kit RED-tris-NTA 2^{nd} generation or Kit RED-NHS 2^{nd} Generation Labeling Kit (NanoTemper Technologies, Munich, Germany). Protein concentration optimization was performed, and final concentrations were 20 nM fluorescently labeled GDP-HRas^{G12V}. Data was acquired in PBS buffer with 0.1% pluronic acid. Data was analyzed with the NanoTemper MO.Affinity Analysis software.

### Measurement of Dissociation Constants (K_{d}) by ITC.

ITC experiments were performed using a Nano ITC isothermal titration calorimeter from TA Instruments. Experiments were performed in reverse-mode by titrating 50 μL of protein solution at a concentration of 300-600 μM into 350 μL of ligand solution at concentrations between 10-50 μM. Stir rate was 200-250 rpm with 16-25 injections, each at 2-3 μL with 150 seconds between each injection. Data fitting was performed using Nano ITCRun software.

### Measurement of Dissociation Constants (K_{d}) by SPR.

SPR experiments were performed using the P4SPR from Affinité Instruments using Ni-NTA immobilization chips and his-tagged protein. Ni-NTA coated surfaces allow the immobilization of his-tagged proteins by chelation of histidine residues to the nickel ion. The sensor chip was inserted into a quad inlet model P4SPR (with 4 independent channels). Once the instrument was turned on, the baseline was stabilized by deionized (DI) water, followed by signal stabilization by the running buffer. His-tagged protein at 10 μg/mL was injected into all 4 channels of the P4SPR and was left to react for 20 min. The sensor chip was then washed with DI water. The lowest concentration of the ligand was injected into the channels of the P4SPR and was left to react for 10 min. The SPR shift was saved. Then, a higher concentration of the ligand was injected, and the sample injection steps were repeated until all 5 concentrations have been added. The KD of the binding interaction between the ligand and protein was determined by using the affinity curve fitting function in the P4SPR Control software.

### Fragment Screening and Hit Confirmation.

Since the fragment library was curated using phosphate buffer, 25 mM sodium phosphate pH 7.4, 150 mM NaCl, 5 mM MgCl₂, 1 mM TCEP-_{d16}, 10% D₂O Fresh TCEP was used for experiments. Screening was performed using ¹H-decoupled 1D ¹⁹F experiment Bruker experiment (zgfhigqn.2) with 64 scans and a relaxation delay of 5 seconds. 50 μM HRas^{G12V} and 240 μM of each fragment in pools (mixtures) were used.

Hit confirmation was performed using the same ¹⁹F experiment as the screen. Ligand-observed 1D ¹H, protein-observed 1D ¹H, ¹H T2-CPMG as well as Saturation Transfer Difference (STD) experiments were also run on the same samples. Ligand-observed 1D ¹H was run using the standard Bruker sequence with excitation sculpting (zgesgp) with a relaxation delay of 10 seconds and 16 scans per spectrum. Protein-observed 1D ¹H was performed using the same sequence with a relaxation delay of 1 second and 256 scans in order to increase the signal-to-noise of the protein resonances, while keeping the experimental time relatively short. The ¹H T2-CPMG experiment employed a modified version of the Bruker 1D ¹H zgesgp experiment with the addition of a CPMG pulse train after the first 90° excitation pulse. Total duration for each spin echo was fixed at 1 ms (τ=500 μs), while the number of echoes in the pulse train was varied according to the total time (T). The number of scans for each spectrum was 4 and eight delay times were run on each sample, ranging from 1 ms to 800 ms.

STD experiments consisted of two independent spectra with offsets of 0 ppm (on resonance) and −20 ppm (off resonance). Subtraction of the on-resonance from the off-resonance spectra resulted in the STD signal. 160 scans were acquired for each spectrum.

### Fragment Library.

Fragment screening has been performed using a library containing 461 fragments that has been designed using cheminformatics parameters such as Rule of Three and PAINS analysis. All fragments have been curated by ¹H and ¹⁹F NMR for structure verification, purity, solubility in phosphate buffer and lack of apparent aggregation under these conditions. Fragments were pooled according to chemical compatibility resulting in 31 different pools to increase screening throughput. The library was provided by NMX Research in Solutions Inc.

(https://www.nmxresearch.com/). Further information about this library is available through Key Organics
(https://www.keyorganics.net/services/bionet-products/fragment-libraries/) as the BIONET Fluorine Fragment Library.

### Nucleotide release assay.

Nucleotide release rates were measured using 1 μM BODIPY-GDP-loaded HRas^{G12V} in 25 mM Tris, pH 7.4, 50 mM NaCl, 1 mM DTT, 10 mM MgCl₂. The nucleotide release reaction was then initiated by addition of either DMSO (control) or compounds in DMSO (across a range of concentrations), SOS^{cat} and unlabeled GTP to final concentrations of 500 nM and 20 μM, respectively. DMSO content was kept constant at 3% in all conditions.

Changes in fluorescence were measured by a fluorescence spectrometer (Tecan Infinite M1000 Pro) in a black 384 well plate (Greiner). Fluorescence was excited at λ=485 nm and emission was measured at λ=510 nm every 30 s for 30 minutes at 28° C. Release rates were extracted by fitting the decrease in fluorescence over time to a single exponential decay. The derived rates were normalized to the DMSO-treated sample and plotted against compound concentration as mean±SEM. The IC₅₀ for each compound was calculated by fitting the normalized rates to a four-parameter dose-response curve.

### Example 81—Results HRas^{G12V}

The results obtained with the compounds in the various assays described above are depicted in Table 21.

Comparative compounds #10005, 10007, 10008, 10009, 10010, and 10011 exhibited low or no binding.

Comparative compound FS-1254 also exhibited low or no binding:

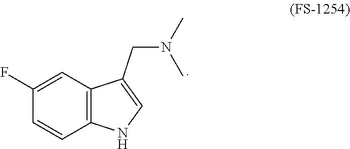

The following compounds (comparative compounds) also exhibited low or no binding: Compounds #10012, 10015, 10017, 10019, 10022, 10023, 10024, 10025, 10026, 10027, 10028, 10029, 10031, 10032, 10033, 10034, 10036, 10037, 10038, 10043, 10044, 10045, 10046, 10047, 10048, 10049, 10050, 10051, 10054, 10055, 10056, 10057, 10060, 10061, 10062, 10063, 10064, 10067, 10070, 10071, 10072, 10073, 10074, 10077, 10079, 10080, 10082, 10083, 10084, 10090, 10092, 10093, 10094, 10096, 10098, 10101, 10102, 10104, 10105, 10107, 10108, 10112, 10113, 10118, 10119, 10121, 10123, 10127, 10133, 10136, 10137, 10140, 10141, 10142, 10143, 10149, 10150, 10151, 10152, 10153, 10155, 10156, 10157, 10158, 10162, 10163, 10164, 10165, 10166, 10168, 10169, 10170, 10171, 10172, 10173, 10174, 10175, 10176, 10177, 10178, 11001, 11004, 11007, 11011, 11013, 11014, 11015, 11016, 11025, 11026, 11041, 11042, 11045, 11058, 11067, 11068, 11069, 11070, 11071, and 11072.

More active compounds included Compounds #10014, 10018, 10035, 10040, 10053, 10066, 10076, 10086, 10095, 10097, 10159, 11005, 11006, 11008, 11032, 11043, 11052 (enantiomer 1 of 11043), 11053 (enantiomer 2 of (11043), 11054, 11055, 11066, and 11073.

Most active compounds included Compounds #10095, 10097, 11005, 11006, 11008, 11032, 11053, and 11073.

### NMR Biophysics and its Utility in Early Hit to Lead for Hard to Drug Targets.

Because of its power to probe the intrinsically weak interactions (K_{d}) between targets and low-molecular weight fragments, NMR fragment-based lead discovery (FBLD) is a very useful biophysical method in early stage drug discovery, especially for challenging target proteins; including those with shallow binding pockets. Since FBLD screens are performed using low-molecular weight compounds (fragments) with lower structural complexity, the same chemical space can be covered using a smaller number of drug-like fragments versus use of large libraries of higher-molecular weight lead-like compounds as in high throughput screening (HTS) campaigns. Therefore, FBLD has a much higher hit rate than HTS using only several hundred to several thousand compounds in comparison with approximately 1 million compounds for HTS. Also, the small size of the compounds screened in FBLD means a better chance to fully match the interactions with small or transient pockets within the target protein, where these less tractable pockets cannot be accessed with standard classical HTS methods. Hits identified (HI) using FBLD typically achieve better ligand efficiency (LE) and better physicochemical properties, thereby permitting the design of more potent and drug-like lead optimizable chemotypes. We expect this methodology to be more advantageous than classical HTS methods for expediting the time from hit to lead (H2L) and allows for rapid access to high quality leads with lower attrition rates in subsequent lead optimization (LO) campaigns toward pre-clinical candidate selection. FBLD screening by NMR involves a “consensus binding” approach where several distinct NMR experiments (DLB, STD, CPMG) are applied along with a free-state aggregation detection method (T2-CPMG) to rank the compounds for their affinity to the receptor (HRAS in this case) whilst simultaneously monitoring the solution behavior and critical quality attributes of each compound. The latter helps to eliminate artifact binders which are highly problematic. We performed a fluorine screen involving 461 compounds that were derived from a highly curated fragment library. Moreover, two-dimensional ¹⁵N HSQC NMR was also used to validate ligand-binding sites on the target protein. In addition, we employed protein-detected biophysical measurements were employed to determine fragment binding affinities to the target protein (K_{d}) by HSQC NMR. From the results, we picked the most interesting hit were picked, FS-1255 that was identified based on consensus binding evidence from all NMR detection methods, and based the lack of free-state aggregation by T2-CPMG. We chose to prioritize on FS-1255 which was the most promising fragment hit. FS-1255 exhibited weak binding affinity, with a ¹⁹F DLB score of 1.77 and a ¹⁹F T2-CPMG of 6.26. The higher-ranking scores on one or two of these affinity descriptors from small molecule screened suggest higher affinity compounds. Those compounds with lower values than, for example FS-1255, suggest lower or no affinity for the receptor.

TABLE 21

| | Exact | | ¹⁹F T2-CPMG | NMR Kd | MST Kd | SPR Kd | ITC Kd |
|---|---|---|---|---|---|---|---|
| Compound # | Mass | ¹⁹F DLB Score | Score | (uM) | (uM) | (uM) | (uM) |
| 10002 | 246.117 | 10.64/3.42 | 19.5/39.8 | 2018-3000 | | | |
| 10003 | 177.059 | 2.8 | 16.04 | | | | |
| 10004 | 285.095 | 5.34 @ 50 uM | 19.49 | 6162-6400 | | | |
| 10005 | 192.106 | 1.52 | 7.25 | | | | |
| 10006 | 256.068 | 3.24 | 19.5 | 10000 | | | |
| 10007 | 178.091 | 1.62 | 5.18 | | | | |
| 10008 | 193.054 | 1.5 | 3.43 | | | | |
| 10009 | 179.038 | 1.36 | 2.28 | | | | |
| 10010 | 222.08 | 1.43 | 2.08 | | | | |
| 10011 | 192.106 | 1.45 | 6.31 | | | | |
| 10012 | 314.123 | 1.32 | 3.96 | | | | |
| 10013 | 276.127 | 2.09 | 12.62 | | | | |
| 10014 | 325.123 | 3.12 | 38.3 | 2323 | | | |
| 10015 | 305.154 | 1.57 | 7.79 | | | | |
| 10016 | 262.28 | 4.63/2.64 | 39.3/15.15 | | | | |
| 10018 | 317.19 | 1.64/1.79/1.93/ | 10.62/11.88/18.24/ | 3027-3700 | | | |
| | | 1.86/1.71 | 9.37/15.13 | | | | |
| 10020 | 318.186 | 1.62 | 14.34 | | | | |
| 10021 | 268.101 | 8.73 @ 50 uM | 38.83 | | | | |
| 10023 | 276.127 | 1.53 | 6.9 | | | | |
| 10024 | 246.117 | 1.06 | 0.01 | | | | |
| 10025 | 250.112 | 1.22 | 1.68 | 6000 | | | |
| 10026 | 264.124 | 1.23 | 3.17 | 10000 | | | |
| 10027 | 276.127 | 1.42 | 3.77 | 7400 | | | |
| 10028 | 289.159 | 1.01 | 1.61 | | | | |
| 10029 | 297.127 | 1.19 | 1.2 | | | | |
| 10030 | 298.112 | 3.05 | 18.47 | | | | |
| 10031 | 314.123 | 1.13 | 1.34 | | | | |
| 10034 | 354.138 | 1.6 | −8.2 | | | | |
| 10035 | 233.096 | 0.91 | −0.11 | 10000+ | | | |
| 10036 | 289.859 | 1.22 | 2.32 | | | | |
| 10037 | 292.122 | 1.25 | 4.07 | | | | |
| 10038 | 292.122 | 1.1 | 2.19 | | | | |
| 10039 | 300.107 | 0.96 | 20.33 | | | | |
| 10040 | 303.138 | 1.64 | 12.69 | 4032-9500 | | | |
| 10041 | 303.138 | 1.86 | 3.92 | 5203-9000 | | | |
| 10042 | 307.112 | 2.72 | 37.91 | | | | |
| 10044 | 317.154 | 1.07 | 0.95 | | | | |
| 10045 | 317.154 | 1.1 | 1.13 | | | | |
| 10046 | 336.074 | 1.02 | 0.79 | | | | |
| 10047 | 339.155 | 1.19 | 3.38 | | | | |
| 10049 | 260.132 | 1.43 | 3.53 | | | | |
| 10050 | 278.107 | 1.22 | 2.95 | | | | |
| 10050 | 278.107 | 1.22 | 2.95 | | | | |
| 10051 | 332.073 | Not soluble | | | | | |
| 10052 | 240.106 | 4.75 | 25.51 | | | | |
| 10053 | 260.132 | 5.85 | 39.43 | 1527 | | | |
| 10054 | 293.096 | Not soluble | | | | | |
| 10055 | 332.073 | Not soluble | | | | | |
| 10056 | 316.102 | Not soluble | | | | | |
| 10057 | 312.127 | Not soluble | | | | | |
| 10058 | 269.096 | 3.07 | 21.6 | | | | |
| 10059 | 312.091 | 3.22 | 18.99 | | | | |
| 10060 | 356.117 | Not soluble | | | | | |
| 10061 | 316.0779 | Not soluble | | | | | |
| 10062 | 292.122 | 1.07 | 1.59 | | | | |
| 10063 | 276.127 | 1.1 | 2.82 | | | | |
| 10064 | 291.174 | 1.16 | 1.74 | | | | |
| 10065 | 275.143 | 2.88 | 22.6 | | | | |
| 10066 | 246.117 | 2.94 | 21.34 | 3507-4000 | | | |
| 10067 | 268.101 | 1.2 | 2.15 | | | | |
| 10068 | 303.175 | 2.43 | 10.72 | | | | |
| 10069 | 274.112 | 2.54 | 21.31 | | | | |
| 10070 | 262.112 | 1.46 | 5.56 | | | | |
| 10071 | 262.112 | 1.35 | 4.49 | | | | |
| 10072 | 286.092 | Aggregated | | | | | |
| 10073 | 317.154 | 1.17 | 2.3 | | | | |
| 10074 | 317.154 | 1.23 | 3.48 | | | | |
| 10075 | 276.127 | 1.77 | 7.09 | | | | |
| 10076 | 260.133 | 3.34 | 37.86 | | | | |
| 10077 | 246.117 | 0.96 | 0.41 | | | | |
| 10078 | 270.0917 | 2.07 | 11.04 | | | | |
| 10079 | 320.154 | 1.23 | 2.25 | | | | |
| 10080 | 355.1332 | 2.67 | −1.66 | | | | |
| 10081 | 262.112 | 2.15 | 15.91 | | | | |
| 10082 | 327.105 | 1.22 | 2.61 | | | | |
| 10083 | 264.091 | 1.14 | 2.8 | | | | |
| 10084 | 264.091 | 1.09 | 1.7 | | | | |
| 10085 | 232.101 | 2.43 | 21.45 | | | | |
| 10086 | 276.127 | 3.04 | 32.13 | 2368-4000 | 1813.5-2084.5 | | |
| 10087 | 290.107 | 2.16 | 13.65 | | | | |
| 10088 | 290.107 | 2.24 | 11.14 | | | | |
| 10089 | 292.1223 | 1.89 | 9.84 | | | | |
| 10090 | 303.138 | 1.51 | 6.28 | | | | |
| 10091 | 303.138 | 1.93 | 7.83 | | | | |
| 10092 | 303.138 | 1.46 | 7.82 | | | | |
| 10093 | 334.17 | 1.35 | 4.89 | | | | |
| 10094 | 342.138 | 3 | 2.5 | | | | |
| 10095 | 355.17 | 4.19 | 33.38 | 36 | 19.1-23.5 | | |
| 10096 | 368.154 | 1.14 | 2.11 | | | | |
| 10097 | 368.165 | 3.84 | 38.49 | 108 | 65-82.9 | 31 | 17-159 |
| 10098 | 384.149 | 1.38 | 5.94 | | | | |
| 10099 | 303.175 | 1.4 | 11.85 | | | | |
| 10100 | 311.143 | 1.73 | 9.66 | | | | |
| 10101 | 335.128 | 1.12 | 1.74 | | | | |
| 10102 | 276.091 | 1.06 | 2.65 | | | | |
| 10103 | 306 | 1.62 | 7.82 | | | | |
| 10104 | 358.144 | 1.14 | 2.49 | | | | |
| 10105 | 308.117 | 1.2 | 2.44 | | | | |
| 10106 | 274.112 | 2.25 | 13.41 | 6428 | | | |
| 10107 | 347.165 | 1.29 | 4.75 | | | | |
| 10108 | 333.154 | 1.34 | 5.72 | | | | |
| 10109 | 274.112 | 3.45 | 15.4 | | | | |
| 10110 | 319.17 | 2.87 | 25.25 | | | | |
| 10111 | 332.19 | 2.23 | 12.96 | | | | |
| 10113 | 276.091 | 1.29 | 2.55 | | | | |
| 10114 | 275.107 | 2.14 | 15.2 | 11068 | | | |
| 10115 | 300.127 | 1.89 | 12.42 | | | | |
| 10116 | 302.143 | 1.88 | 13.95 | | | | |
| 10117 | 333.15 | 1.54 | 10.14 | | | | |
| 10118 | 333.15 | 1.15 | 2.67 | | | | |
| 10119 | 333.15 | 0.87 | −0.46 | | | | |
| 10120 | 276.127 | 2.34 | 13.94 | | | | |
| 10121 | 334.133 | 1.26 | 5.23 | | | | |
| 10122 | 288.127 | 1.68 | 9.62 | | | | |
| 10123 | 304.122 | 1.5 | 2.37 | | | | |
| 10124 | 290.143 | 1.82 | 12.49 | | | | |
| 10125 | 304.122 | 1.56 | 10.15 | | | | |
| 10126 | 332.154 | 4.22 | 13.7 | | | | |
| 10127 | 318.138 | 1.3 | 4.35 | | | | |
| 10128 | 332.154 | 2.85 | 13.67 | | | | |
| 10129 | 304.122 | 1.27 | 3.92 | | | | |
| 10130 | 346.17 | 2.09 | 12.1 | | | | |
| 10131 | 276.127 | 2.17 | 20.77 | | | | |
| 10132 | 332.154 | 2.01 | 13.29 | | | | |
| 10133 | 304.122 | 1.63 | 2.85 | | | | |
| 10134 | 290.143 | 2.1 | 13.32 | | | | |
| 10135 | 332.153 | 2 | 12.28 | | | | |
| 10136 | 338.143 | 1.55 | 6.73 | | | | |
| 10137 | 317.154 | 2.02 | 6.16 | | | | |
| 10138 | 274.112 | 1.98 | 14.25 | | | | |
| 10139 | 317.154 | 1.93 | 4.54 | | | | |
| 10140 | 334.133 | 1.49 | 5.98 | | | | |
| 10141 | 375.196 | 1.21 | 3.15 | | | | |
| 10142 | 347.165 | 1.11 | 2.43 | | | | |
| 10143 | 246.117 | 0.89 | 0.9 | | | | |
| 10144 | 247.112 | 1.93 | 9.44 | | | | |
| 10145 | 275.101 | 1.64 | 16.13 | | | | |
| 10146 | 289.123 | 1.56 | 9.75 | | | | |
| 10147 | 292.122 | 2.3 | 19.87 | | | | |
| 10148 | 292.122 | 1.87 | 12.46 | | | | |
| 10149 | 304.122 | 1.56 | 2.26 | | | | |
| 10150 | 306.102 | 1.24 | 2.07 | | | | |
| 10151 | 306.102 | 1.29 | 3.24 | | | | |
| 10152 | 306.102 | 1.03 | 3.54 | | | | |
| 10153 | 306.102 | 1.39 | 3.22 | | | | |
| 10154 | 306.138 | 1.62 | 8.16 | | | | |
| 10155 | 306.138 | 1.49 | 8.16 | | | | |
| 10156 | 333.149 | 1.42 | 3.43 | | | | |
| 10157 | 333.149 | 1.04 | 3.58 | | | | |
| 10158 | 319.133 | 0.93 | 2.14 | | | | |
| 10159 | 319.17 | 3.93 | 19.26 | 2952-3427 | | | |
| 10160 | 319.17 | 6.24 | NA | 1284 | | | |
| 10161 | 332.154 | 2.07 | 7.87 | | | | |
| 10162 | 333.149 | 1.65 | 5.5 | | | | |
| 10163 | 333.149 | 1.61 | 7.3 | | | | |
| 10164 | 333.149 | 1.61 | 5.59 | | | | |
| 10165 | 319.133 | 1.58 | 5.28 | | | | |
| 10166 | 319.133 | 1.55 | 7.19 | | | | |
| 10167 | 333.149 | 3.62 | 5.07 | | | | |
| 10168 | 334.133 | 1.43 | 4.84 | | | | |
| 10169 | 334.133 | 1.6 | 7.14 | | | | |
| 10170 | 359.165 | 1.52 | 6.11 | | | | |
| 10171 | 348.149 | 1.19 | 7.62 | | | | |
| 10172 | 246.117 | 0.93 | 0.11 | | | | |
| 10173 | 319.133 | 1.33 | 3.79 | | | | |
| 10174 | 247.112 | 1.14 | 2.32 | | | | |
| 10175 | 320.117 | 1.19 | 2.15 | | | | |
| 10176 | 246.117 | 1.01 | 0.19 | | | | |
| 10177 | 348.149 | 1.12 | 7.14 | | | | |
| 10178 | 320.117 | 1.11 | 2.74 | | | | |
| 11001 | 207.2 | 1.76 | 6.77 | | | | |
| 11002 | 335.15 | 2.58 | 13.63 | | | | |
| 11003 | 221.08 | 2.23 | 11.18 | | | | |
| 11004 | 321.14 | 0.91 | 1.91 | | | | |
| 11005 | 274.15 | 3.19 | 16.59 | | | | |
| 11006 | 232.06 | 3.43 | 24.24 | | | | |
| 11007 | 204.11 | 1.76 | 6.67 | | | | |
| 11008 | | 6.41 | 38.4 | | | | |
| 11009 | | 0.99 | 0.07 | | | | |
| 11010 | | 1.07 | 0 | | | | |
| 11011 | | 1.52 | 5.22 | | | | |
| 11012 | | 2.58 | 13.63 | | | | |
| 11013 | | 0.4 | 1.74 | | | | |
| 11014 | | 1.44 | 3.32 | | | | |
| 11015 | | 0.36 | −10.75 | | | | |
| 11016 | | 1.74 | 5.57 | | | | |
| 11017 | 415.23 | 1.86 | 16.89 | | | | |
| 11018 | 590.19 | 2.51 | 9.96 | | | | |
| 11019 | 330.17 | 2.2 | 12.9 | | | | |
| 11020 | 379.17 | 2.23 | 15.24 | | | | |
| 11021 | 308.13 | 3.35 | 39.09 | | | | |
| 11022 | 326.12 | 3.43 | 5.71 | | | | |
| 11023 | 365.15 | 3.07 | 16.39 | | | | |
| 11024 | 357.19 | 1.89 | 9.57 | | | | |
| 11025 | 322.15 | 1 | −2.12 | | | | |
| 11026 | 358.13 | 2.02 | −1.86 | | | | |
| 11027 | 344.42 | 2.48 | 13.23 | | | | |
| 11028 | 357.19 | 2.5 | 39.3 | | | | |
| 11029 | 371.2 | 1.93 | 10.24 | | | | |
| 11030 | 350.15 | 2.9 | 21.82 | | | | |
| 11031 | 381.11 | 1.97 | 38.39 | | | | |
| 11032 | 393.16 | 4.73 | <LLOQ | <0 | 0.772 | | 0.387-0.517 |
| 11033 | 271.11 | 6.06 | | | | | |
| 11034 | 304.12 | 1.7 | 39.41 | | | | |
| 11035 | 350.12 | 5.34 | | | | | |
| 11036 | 301.36 | 1.76 | 19.33 | | | | |
| 11037 | 315.17 | 2.12 | 16.77 | | | | |
| 11038 | 315.17 | 2.37 | | | | | |
| 11039 | 232.06 | 3.59 | 39.34 | | | | |
| 11040 | 232.06 | 3.49 | | | | | |
| 11041 | 385.11 | 1.65 | 3.61 | | | | |
| 11042 | 385.11 | 1.56 | 5.62 | | | | |
| 11043 | 301.09 | 3.27 | 20.23 | | | | |
| 11044 | 301.09 | 2.77 | 16.24 | | | | |
| 11045 | 329.12 | 16.69 | | | | | |
| 11046 | 315.17 | 3.58 | | | | | |
| 11047 | 329.19 | 2.79 | 22.81 | | | | |
| 11048 | 329.19 | 3.29 | | | | | |
| 11049 | 192.23 | Non conclusive | | | | | |
| 11050 | 207.21 | Non conclusive | | | | | |
| 11051 | 174.24 | Lacks F atom | | | | | |
| 11052 | 301.29 | Non conclusive | | | | | |
| 11053 | 301.29 | 2.29 | 25.91 | | | | |
| 11054 | 299.3 | Non conclusive | | | | | |
| 11055 | 299.3 | Non conclusive | | | | | |
| 11056 | 319.28 | Non conclusive | | | | | |
| 11057 | 313.32 | Non conclusive | | | | | |
| 11058 | 403.35 | Non conclusive | | | | | |
| 11059 | 301.29 | Non conclusive | | | | | |
| 11060 | 298.31 | Non conclusive | | | | | |
| 11061 | 378.19 | Non conclusive | | | | | |
| 11062 | 292.31 | Non conclusive | | | | | |
| 11063 | 314.31 | Non conclusive | | | | | |
| 11064 | 188.23 | Lacks F atom | | | | | |
| 11065 | 206.22 | Non conclusive | | | | | |
| 11066 | 358.38 | 0.91 | −0.25 | | | | |
| 11067 | 429.317 | Lacks F atom | | | | | |
| 11068 | 384.866 | Lacks F atom | | | | | |
| 11069 | 209.632 | Lacks F atom | | | | | |
| 11070 | 384.866 | Lacks F atom | | | | | |
| 11071 | 384.866 | Lacks F atom | | | | | |
| 11072 | 402.856 | 0.08 | 1.02 | | | | |
| 11073 | 402.856 | <LLOQ | <LLOQ | | | | |
| 11074 | 244.177 | Lacks F atom | | | | | |
| FS-1254 | 192.106 | 1.15 | 0.74 | | | | |
| (comp) | | | | | | | |
| FS-1255 | 192.106 | 1.7/1.65 | 6.26/5.22 | 9834-12000 | | | |
| (comp) | | | | | | | |

### Example 82—Results Wild Type HRAS

The activity of compounds of the invention regarding wild type HRAS was measured in the same as in Example 80. The results obtained with the compounds in the various assays described above are depicted in Table 2.

TABLE 22

| Compound | Exact | ¹⁹F | ¹⁹F T2-CPMG | NMR |
|---|---|---|---|---|
| # | Mass | DLB Score | Score | Kd (uM) |
| 10018 | 317.19 | | | 5184 |
| 10095 | 355.17 | 4.57 | <LLOQ | |
| 10097 | 368.165 | 7.61 | <LLOQ | |
| 11032 | 393.16 | 5.76 | <LLOQ | |
| FS-1255 | 192.106 | | | 6068 |

### Example 83—Results KRAS^{G12D-GDP}

The activity of compounds of the invention regarding KRAS^{G12D-GDP} was measured in the same as in Example 80. The results obtained with the compounds in the various assays described above are depicted in Table 23.

The most active compounds included Compounds #, 10018, 10040, 10086, and 11066. Prior art compound #10002 is also active.

TABLE 23

| Compound | Exact | ¹⁹F | ¹⁹F T2-CPMG | 1H 1D PO |
|---|---|---|---|---|
| # | Mass | DLB Score | Score | NMR Kd (uM) |
| 10002 | 246.117 | 3.74 | >LOQ | 2204 |
| 10004 | 285.095 | 2.06 | 9.41 | |
| 10014 | 325.123 | 2.46 | >LOQ | |
| 10018 | 317.19 | 1.73 | 12.20 | 5019-6254 |
| 10021 | 268.101 | 2.62 | >LOQ | |
| 10040 | 303.138 | 1.63 | 7.49 | 5201 |
| 10053 | 260.132 | 4.89 | >LOQ | |
| 10066 | 246.117 | 2.22 | 9.14 | |
| 10086 | 276.127 | 3.33 | >LOQ | 1897-4031 |
| 10095 | 355.17 | 3.56 | >LOQ | |
| 10097 | 368.165 | 4.36 | >LOQ | |
| 11003 | 221.08 | 2.20 | 14.19 | |
| 11006 | 274.15 | 2.70 | 10.14 | |
| 11008 | 232.06 | 2.68 | >LOQ | 10226 |
| 11012 | 368.411 | 5.00 | >LOQ | |
| FS-1255 | 218.274 | 1.51 | 3.62 | |

### Example 84—Proliferation Assays

### Cell Lines and Cell Culture

Bladder cancers (T24) and (5637) and primary bladder epithelial/normal cells (BdEC) were obtained from American Type Culture Collection (ATCC). T24 cells were cultured in (McCoy's) medium, and 5637 cells in (RPMI-1640), supplemented with 10% FBS. BdEC cells were cultured in healthy bladder epithelial basal medium supplemented with a growth kit as recommended by the manufacturer. Cells were incubated in a humidified atmosphere of 5% CO₂ at 37° C. to ensure growth and viability.

### Proliferation Assay

Antiproliferative effects were evaluated by using MTT (3-(4,5-dimethylthiazolyl-2)-2,5-diphenyltetrazolium bromide). When cells reached 80-90% of confluency, they were cultured overnight in 96 well plates, in a humidified atmosphere of 5% CO₂ at 37° C. Cells were then starved with media without FBS for 4 hours. After starvation, cells were treated with various concentrations of each compound diluted in fresh media without FBS for 72 h. After incubation, 20 μl of MTT solution, prepared at (5 mg/mL) was added to each well and incubated for 4 h in the dark at 37° C. DMSO was used as a vehicle control to normalize inhibitory response. Data was fitted to dose-response curves in order to estimate inhibitory concentration (IC₅₀) values.

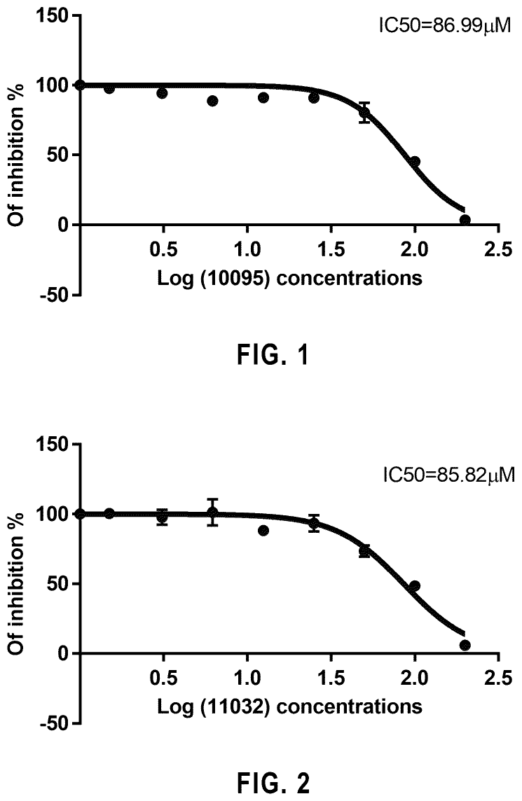

FIG. 1 shows the activity of compound #10095 on healthy bladder cells BdEC.

FIG. 2 shows the activity of compound #11032 on healthy bladder cells BdEC.

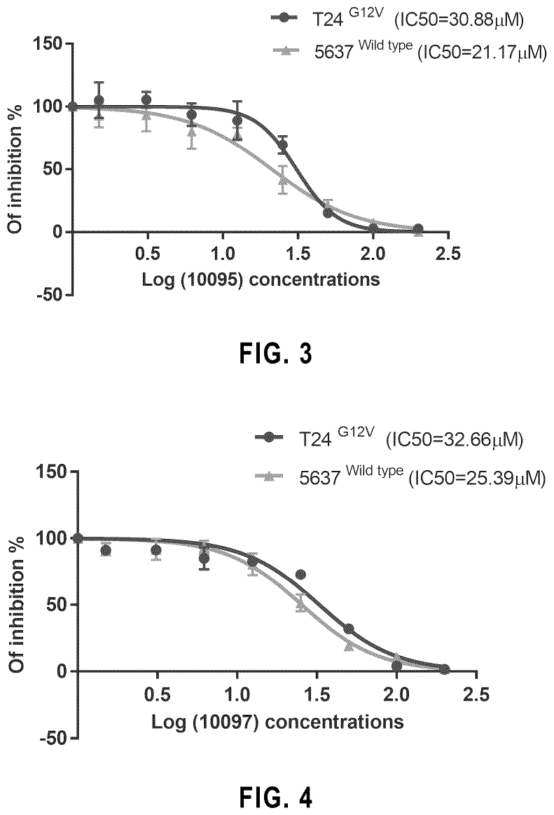

FIG. 3 shows the activity of compound #10095 on Bladder Cancer Cells T24 and 5637.

FIG. 4 shows the activity of compound #10097 on Bladder Cancer Cells T24 and 5637.

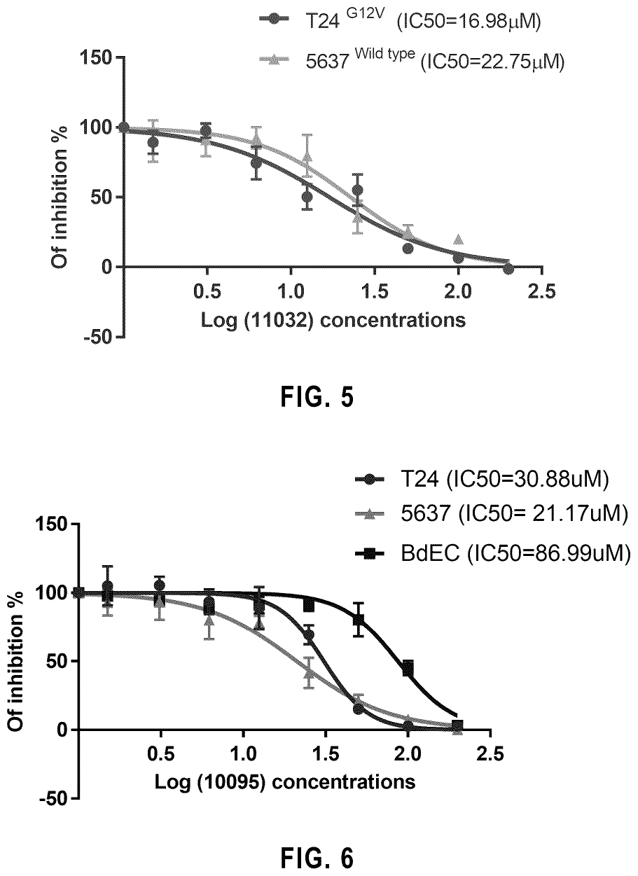

FIG. 5 shows the activity of compound #11032 on Bladder Cancer Cells T24 and 5637.

FIG. 6 shows the activity of compound #10095 on healthy bladder cells BdEC and Bladder Cancer Cells T24 and 5637.

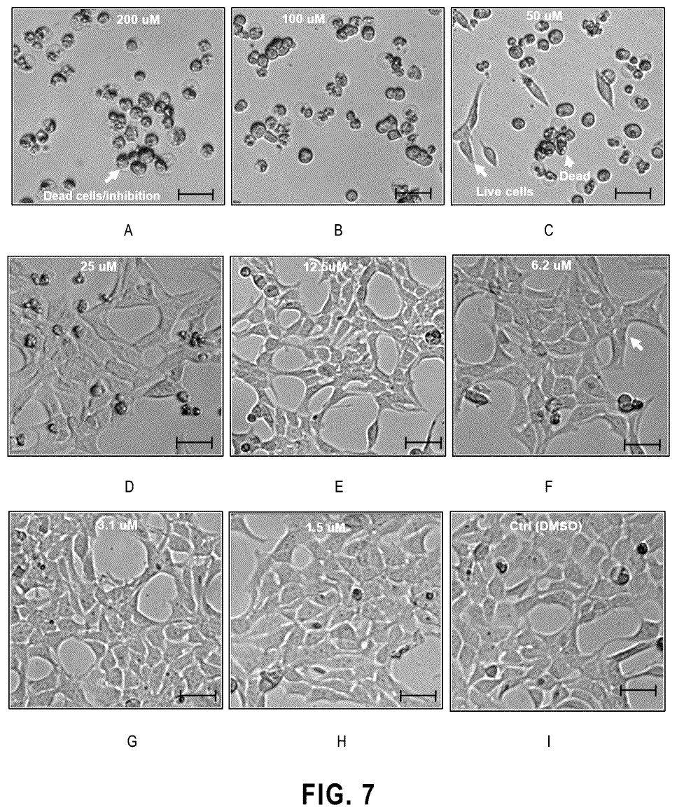

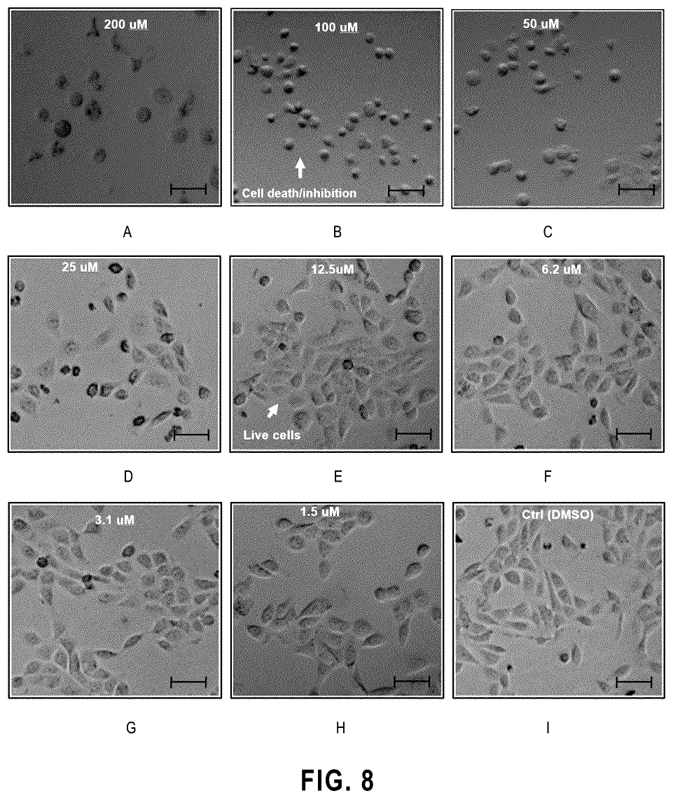

FIG. 7 A to I shows the morphology of bladder cancer cells (T24) following treatment with decreasing concentrations of 10095, A: 200 μM, B: 100 μM, C: 50 μM, D: 25 μM, E: 12.5 μM, F: 6.2 μM, G: 3.1 μM, H: 1.5 μM, and I: control.

FIG. 8 A to I shows the morphology of bladder cancer cells (5637) following treatment with decreasing concentrations of 10095, A: 200 μM, B: 100 μM, C: 50 μM, D: 25 μM, E: 12.5 μM, F: 6.2 μM, G: 3.1 μM, H: 1.5 μM, and I: control.

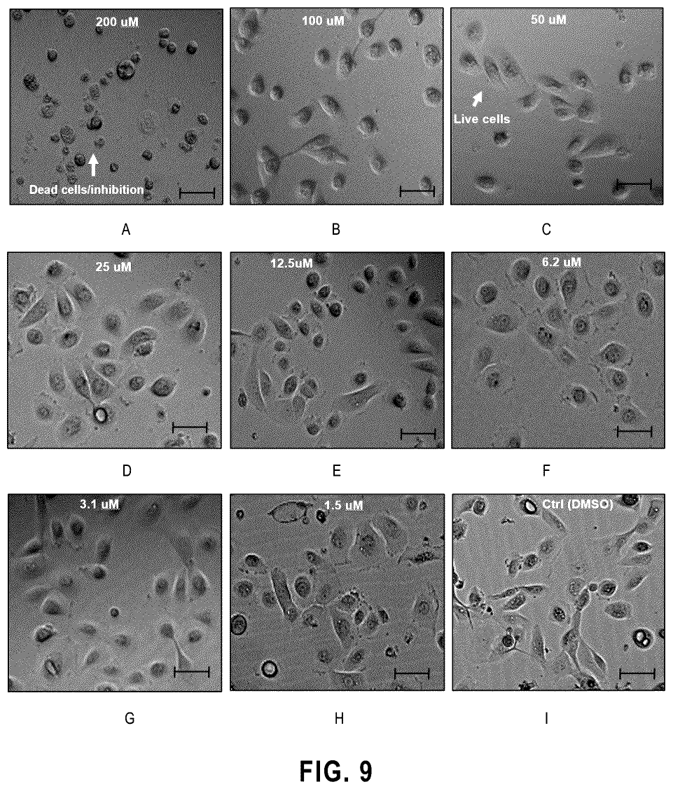

FIG. 9 A to I shows the morphology of bladder epithelial healthy (BdEC) cells following treatment with decreasing concentrations of 10095, A: 200 μM, B: 100 μM, C: 50 μM, D: 25 μM, E: 12.5 μM, F: 6.2 μM, G: 3.1 μM, H: 1.5 μM, and I: control.

The scope of the claims should not be limited by the preferred embodiments set forth in the examples but should be given the broadest interpretation consistent with the description as a whole.

### REFERENCES

The present description refers to a number of documents. the content of which is herein incorporated by reference in their entirety. These documents include, but are not limited to, the following:
CN106083830B
CN107311933A
EP1643991B1
EP1956910B1
   EP2671575A1
   EP2698367A1
   EP3601267A1
   EP948483B1
   U.S. Ser. No. 10/487,078B2
   U.S. Ser. No. 10/501,421B1
   U.S. Ser. No. 10/723,738B2
   US20200147058A1
   US20200247762A1
   US20200308165A1
   U.S. Pat. No. 6,403,581B1
   U.S. Pat. No. 8,575,191B2
   U.S. Pat. No. 8,791,118B2
   WO2015038704A1
   WO2015184349A2
   WO2016176338A1
   WO2019110751A1
   WO2019215203A1
   WO2020173935A1
   WO2020178282A1
   Abbott et al., Discovery of Aminopiperidine Indoles That Activate the Guanine Nucleotide Exchange Factor SOS1 and Modulate RAS Signaling. Journal of Medicinal Chemistry. 2018. Volume 61. Issue 14. Pages 6002 to 6017
   rito et al., Targeting KRAS Oncogene in Colon Cancer Cells with 7-Carboxylate Indolo[3.2-b]quinoline Tri-Alkylamine Derivatives. PLoS ONE 10(5): e0126891.
   Fang et al., Inhibition of K-RAS4B by a Unique Mechanism of Action: Stabilizing Membrane-Dependent Occlusion of the Effector-Binding SiteCell Chemical Biology. 2018. Volume 25. Issue 11. Pages 1-10
   Gupta et al., Multi-target. ensemble-based virtual screening yields novel allosteric KRAS inhibitors at high success rate. Chemical Biology & Drug Design. 2019. Volume 94. Issue 2. Pages 1441 to 1456
   Hodges et al., Discovery and Structure-Based Optimization of Benzimidazole-Derived Activators of SOS1-Mediated Nucleotide Exchange on RAS. Journal of Medicinal Chemistry. 2018. Volume 61. Issue 19. Pages 8875 to 8894
   Li et al., Benzimidazolones and indoles as non-thiol farnesyltransferase inhibitors based on tipifarnib scaffold: synthesis and activity. Bioorganic & Medicinal Chemistry Letters. 2005. Volume 15. Issue 11. pages 2910 to 2922
   Maurer et al., Small-molecule ligands bind to a distinct pocket in Ras and inhibit SOS-mediated nucleotide exchange activity. PNAS. 2012. Volume 109. Issue 14. Pages 5299 to 5304
   Mori et al., Improved Sensitivity of HSQC Spectra of Exchanging Protons at Short Interscan Delays Using a New Fast HSQC (FHSQC) Detection Scheme That Avoids Water Saturation. Journal of Magnetic Resonance. Series B. Volume 108. Issue 1. July 1995. Pages 94-98
   A Comparative Analysis of Individual RAS Mutations in Cancer Biology. Front. Oncol., 18 Oct. 2019
   Munoz-Maldonado et al., Shimomura et al., Drug library screen reveals benzimidazole derivatives as selective cytotoxic agents for KRAS-mutant lung cancer. Cancer Letters. 2019. Volume 451. Pages 11 to 22
   Shin et al., Discovery of N-(1-Acryloylazetidin-3-yl)-2-(1H-indol-1-yl)acetamides as Covalent Inhibitors of KRAS^{G12C}. ACS Medicinal Chemistry Letters. 2019. Volume 10. Issue 9. Pages 1302 to 1308
   Sun et al., Discovery of Small Molecules that Bind to K-Ras and Inhibit Sos-Mediated Activation. Angewandte Chemie International Edition. 2012. Volume 51. Issue 25. Pages 6140 to 6143
   Williamson. Using chemical shift perturbation to characterise ligand binding. Progress in Nuclear Magnetic Resonance Spectroscopy, Volume 73, August 2013, Pages 1-16.
   Zhao et al., Diverse alterations associated with Resistance to KRAS(G12C) inhibition, Nature, 599, 679-683 (2021).
   Welsch et al., Multivalent Small-Molecule Pas-RAS Inhibitors, Cell, 168, 878-889, 2017.

1. A compound of formula (I): 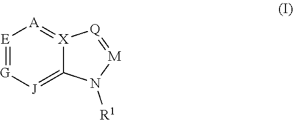
wherein:
R¹ represents H, alkoxycarbonyl, alkylcarbonyl, Cₙ-alkyl, wherein n is 2 or more, or carbamoyl,
A represents —C(R²)═,
E represents —N═ or —C(R³)
G represents —N═ or —C(R⁴)
J represents —C(R⁵)═,
X represents —C(R⁶)═,
M represents —N═ or —C(R⁷)═,
Q represents —N═ or —C(R⁸)═,
with the proviso that no more than four (4) of A, E, G, J, X, M, and Q represent —N═,
each of R⁴, R⁵, R⁶, and R⁸ independently represents H, a halogen atom, alkyl, alkenyl, alkynyl, alkenynyl, hydroxyl, —OR⁹, or -L-R¹⁰,
R² represents H, a halogen atom, alkyl, alkenyl, alkynyl, alkenynyl, alkyl-N(R⁹)₂, alkenyl-N(R⁹)₂, alkynyl-N(R⁹)₂, alkenynyl-N(R⁹)₂, hydroxyl, or —OR⁹,
R³ represents H, alkyl, alkenyl, alkynyl, alkenynyl, alkyl-N(R⁹)₂, alkenyl-N(R⁹)₂, alkynyl-N(R⁹)₂, alkenynyl-N(R⁹)₂, hydroxyl, or —OR⁹,
R⁷ represents H, a halogen atom, Cₙ-alkyl, wherein n is 2 or more, alkenyl, alkynyl, alkenynyl, alkyl-N(R⁹)₂, alkenyl-N(R⁹)₂, alkynyl-N(R⁹)₂, alkenynyl-N(R⁹)₂, hydroxyl, or —OR⁹,
R⁹ represents H, alkyl, alkenyl, alkynyl, or alkenynyl, each of the alkyl, alkenyl, alkynyl, and alkenynyl being optionally substituted with R¹⁶,
with the proviso that the compound of formula (I) comprises exactly one or exactly two -L-R¹⁰ group(s) identical or different from one another,
each L independently represents a covalent bond or a chain comprising any combination of the following:
up to two —N(R¹¹)— group,
up to one —C(═O)—, —O—C(═O)—, —C(═O)—O—, —SO₂—, or —SO— group, and
up to five —C(R¹²)₂— groups, wherein the —C(R¹²)₂— groups can be adjacent to one another or separated by —N(R¹¹)—, —C(═O)—, and/or —S(═O)₂ groups,
wherein, in each -L-R¹⁰ group independently:
E) R¹⁰ represents:
a 6-membered cycle selected from cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl, each of which comprising one or more nitrogen ring atoms as sole heteroatoms, and each of which being independently unsubstituted or substituted with:
a halogen atom, —CN, hydroxyl, —N(R¹⁵)₂, —N(R¹⁵)—C(═O)—R¹⁸, —C(═O)—N(R¹⁵)₂, —C(═O)—R¹⁷, —C(═O)—OR¹⁷, —OR¹⁵, —O—C(═O)—R¹⁷, —SO₂—R¹⁵, —SO—R¹⁵, —N(R¹⁵)₂—SO₂—R¹⁵, or —N(R¹⁵)₂—SO—R¹⁵, or alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, or heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R¹⁶,
or
aryl or heteroaryl, each of which independently unsubstituted or substituted with, in a position other than in the position immediately next to the ring atom attached to L:
alkyl-N(R¹⁵)₂, alkenyl-N(R¹⁵)₂, alkynyl-N(R¹⁵)₂, alkenynyl-N(R¹⁵)₂, —N(R¹⁵)₂, —N(R¹⁵)—C(═O)—R¹⁵, —C(═O)—N(R¹⁵)₂, —C(═O)—R¹⁵, —C(═O)—OR¹⁷, —OR¹⁸, —O—C(═O)—R¹⁵, —SO₂—R¹⁵, —SO—R¹⁵, —N(R¹⁵)₂—SO₂—R¹⁵, —N(R¹⁵)₂—SO—R¹⁵, or
cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of independently unsubstituted or substituted with one or more R¹⁶,
with the proviso that the aryl or heteroaryl are substituted with no more than one —OR¹⁸ group, and
with the proviso that -L-R¹⁰ is not 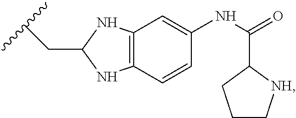 [SMILES: *CC1Nc2ccc(NC(=O)C3CCCN3)cc2N1]
L represents a chain of at most 5 atoms in length,
each R¹¹ independently represents H, or
alkyl, alkenyl, alkynyl, or alkenynyl, each of which independently unsubstituted of substituted with one or more R³⁰,
each R¹² independently represents H, —C≡N, —N(R¹⁵)₂, -T-COOR¹⁴, or
alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰,
F) R¹⁰ represents —COOH,
L represents a chain that is at most 3 atoms in length, and that is not —CH₂—CH(NH₂)—,
any R¹¹ independently represents H, or
alkyl, alkenyl, alkynyl, or alkenynyl, each of which independently unsubstituted or substituted with one or more R³⁰, and
any R¹² independently represents H, —C≡N, —N(R¹⁵)₂, —C(═O)—R¹⁵, or
alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰,
G) R¹⁰ is attached to a nitrogen atom of a —N(R¹¹)— group that ends the chain in L, and R¹⁰ and the R¹¹ of the —N(R¹¹)— group that ends the chain in L together with the nitrogen atom to which they are attached form a heteroaryl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl, each of which independently unsubstituted or substituted with:
a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR²⁰, —OR¹⁵, —O—C(═O)—R¹⁵, —SO₂—R¹⁵, —SO—R¹⁵, —N(R¹⁵)₂—SO₂—R¹⁵, —N(R¹⁵)₂—SO—R¹⁵, or alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R²¹,
with the proviso that when R¹⁰ and R¹¹ together with the nitrogen atom to which they are attached form morpholinyl, the morpholinyl is free of a substituent containing a —C(═O)— group,
with the proviso that when R¹⁰ and R¹¹ together with the nitrogen atom to which they are attached form piperazinyl substituted with a —C(═O)—O—CH₃ group, the —C(═O)—O—CH₃ group is in position 2, with the proviso that when R¹⁰ and R¹¹ together with the nitrogen atom to which they are attached form piperazinyl N-substituted with —O-heterocycloalkyl, the heterocycloalkyl is other than oxalanyl,
the chain in L is at most 4 atoms in length and ends with the —N(R¹¹)— group to which R¹⁰ is attached,
with the proviso that -L-R¹⁰ is not 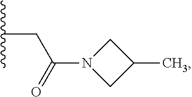 [SMILES: *CC(=O)N1CC(C)C1]
any other R¹¹ independently represents H, or
alkyl, alkenyl, alkynyl, or alkenynyl, each of which independently unsubstituted or substituted with one or more R³¹ and
any R¹² independently represents H, —C≡N, —N(R¹⁵)₂, or
alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, eterocycloalkenynonyl, aryl, or heteroaryl, each of which independently unsubstituted or substituted with one or more R³¹,
with the proviso that when one R¹² on a given carbon atom is methyl, any other R¹² on said given carbon atom is other than methyl,
or
H) R¹⁰ represents H, a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR¹⁵, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵, or alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each independently unsubstituted or substituted with one or more R¹⁶,
the chain in L is at most 3 atoms in length, contains at least one —C(R¹²)₂— group, and ends with a —N(R¹¹)— group,
the R¹¹ of the —N(R¹¹)— group that ends the chain in L and one R¹² of said at least one —C(R¹²)₂— group together with the one or more atoms to which they are attached and any atom(s) between said one or more atoms form heterocycloalkyl, heterocycloalkanonyl, heterocycloalkenonyl, heterocycloalkynonyl, heterocycloalkenynonyl, or heteroaryl, each of which being independently unsubstituted or substituted with:
a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR¹⁵, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵, alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which being independently unsubstituted or substituted with one or more R¹⁶,
with the proviso that when said one R¹¹ and said one R¹² form pyrrolidinyl, the pyrrolidinyl is substituted,
with the proviso that when the heterocycloalkyl is piperidinyl, R¹⁰ is other than 2,4-difluorophenylcarbonyl substituent,
any other R¹¹ independently represent H or
alkyl, alkenyl, alkynyl, alkenynyl, each of which independently unsubstituted or substituted with one or more R³¹, and
any other R¹² independently represent H, —C≡N, or —N(R¹⁵)₂,
alkyl, alkenyl, alkynyl, or alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³¹,
with the proviso that -L-R¹⁰ is not 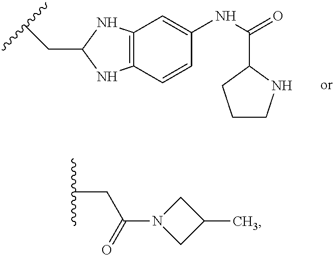
wherein R¹⁴ represents H, alkyl, alkenyl, alkynyl, or alkenynyl,
wherein T represents a covalent bond, alkylene, alkenylene, alkynylene, or alkenynylene,
wherein each R³⁰ independently represents:
a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR¹⁵, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵,
alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R¹⁶,
wherein each R³¹ independently represents:
a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR²⁰, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵,
alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R¹⁶,
wherein each R¹⁵ independently represents H, or
alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R¹⁶
wherein each R¹⁷ independently represents cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, aryl, or heteroaryl, each of which independently unsubstituted or substituted with one or more R¹⁶,
wherein each R²⁰ independently represents alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, aryl, or heteroaryl, or, each of which independently unsubstituted or substituted with one or more R¹⁶
wherein each R¹⁸ independently represents H, or
alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, aryl, or heteroaryl, each of which independently unsubstituted or substituted with one or more R¹⁹,
wherein each R¹⁶ independently represents a halogen atom, alkoxy, alkylcarbonyl, alkenylcarbonyl, alkynylcarbonyl, alkenynylcarbonyl, alkoxycarbonyl, alkyl, alkenyl, alkynyl, alkenynyl, carbamoyl, alkylcarbamoyl, alkenylcarbamoyl, alkynylcarbamoyl, alkenynylcarbamoyl, aryl (optionally substituted with one or more halogen atoms), heteroaryl (optionally substituted with one or more halogen atoms), —CN, carboxyl, hydroxyl, amido, alkylamido, dialkylamido, alkenylamido, dialkenylamido, alkynylamido, dialkynylamido, alkenynylamido, dialkenynylamido, amino, alkylamino, dialkylamino, alkenylamino, dialkenylamino, alkynylamino, dialkynylamino, alkenynylamino, dialkenynylamino, alkylsulfonylamino, alkenylsulfonylamino, alkynylsulfonylamino, or alkenynylsulfonylamino,
wherein each R¹⁹ independently represents a halogen atom, alkylcarbonyl, alkenylcarbonyl, alkynylcarbonyl, alkenynylcarbonyl, alkoxycarbonyl, alkyl, alkenyl, alkynyl, alkenynyl, carbamoyl, alkylcarbamoyl, alkenylcarbamoyl, alkynylcarbamoyl, alkenynylcarbamoyl, aryl (optionally substituted with one or more halogen atoms), heteroaryl (optionally substituted with one or more halogen atoms), —CN, carboxyl, hydroxyl, amido, alkylamido, dialkylamido, alkenylamido, dialkenylamido, alkynylamido, dialkynylamido, alkenynylamido, dialkenynylamido, amino, alkylamino, dialkylamino, alkenylamino, dialkenylamino, alkynylamino, dialkynylamino, alkenynylamino, dialkenynylamino, alkylsulfonylamino, alkenylsulfonylamino, alkynylsulfonylamino, or alkenynylsulfonylamino
wherein each R²¹ independently represents a halogen atom, alkoxy, alkylcarbonyl, alkenylcarbonyl, alkynylcarbonyl, alkenynylcarbonyl, alkoxycarbonyl, alkyl, alkenyl, alkynyl, alkenynyl, carbamoyl, alkylcarbamoyl, alkenylcarbamoyl, alkynylcarbamoyl, alkenynylcarbamoyl, aryl (optionally substituted with one or more halogen atoms), heteroaryl (optionally substituted with one or more halogen atoms), —CN, hydroxyl, amido, alkylamido, dialkylamido, alkenylamido, dialkenylamido, alkynylamido, dialkynylamido, alkenynylamido, dialkenynylamido, amino, alkylamino, dialkylamino, alkenylamino, dialkenylamino, alkynylamino, dialkynylamino, alkenynylamino, dialkenynylamino, alkylsulfonylamino, alkenylsulfonylamino, alkynylsulfonylamino, or alkenynylsulfonylamino,
or a pharmaceutically acceptable salt, ester, solvate, isomer, or tautomer thereof.

2. The compound of claim 1, comprising exactly one -L-R¹⁰ group.

3. The compound of claim 1, comprising exactly two -L-R¹⁰ group.

4. The compound of any one of claims 1 to 3, wherein R¹ represents H.

5. The compound of any one of claims 1 to 4, wherein A represents —CH═.

6. The compound of any one of claims 1 to 5, wherein E represents —C(R³)═, preferably —C(H)═.

7. The compound of any one of claims 1 to 6, wherein G represents —C(R⁴)═, preferably wherein R⁴ represents a halogen atom, more preferably F.

8. The compound of any one of claims 1 to 7, wherein J represents —CH═.

9. The compound of any one of claims 1 to 8, wherein X represents —C(H)═.

10. The compound of any one of claims 1 to 9, wherein M represents —C(R⁷)═, wherein R⁷ preferably represents H.

11. The compound of any one of claims 1 to 9, wherein M represents —N═.

12. The compound of any one of claims 1 to 11, wherein Q represents —C(R⁸)═, preferably —C(-L-R¹⁰)═.

13. The compound of any one of claims 1 to 12, wherein A)
R¹⁰ represents:
a 6-membered cycle selected from cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl, each of which comprising one or more nitrogen ring atoms as sole heteroatoms, and each of which being independently unsubstituted or substituted with
a halogen atom, —CN, hydroxyl, —N(R¹⁵)₂, —N(R¹⁵)—C(═O)—R¹⁸, —C(═O)—N(R¹⁵)₂, —C(═O)—R¹⁷, —C(═O)—OR¹⁷, —OR¹⁵, —O—C(═O)—R¹⁷, —SO₂—R¹⁵, —SO—R¹⁵, —N(R¹⁵)₂—SO₂—R¹⁵, or —N(R¹⁵)₂—SO—R¹⁵, or
alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, or heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R¹⁶,
or
aryl or heteroaryl, each of which independently unsubstituted or substituted with, in a position other than in the position immediately next to the ring atom attached to L:
alkyl-N(R¹⁵)₂, alkenyl-N(R¹⁵)₂, alkynyl-N(R¹⁵)₂, alkenynyl-N(R¹⁵)₂, —N(R¹⁵)₂, —N(R¹⁵)—C(═O)—R¹⁵, —C(═O)—N(R¹⁵)₂, —C(═O)—R¹⁵, —C(═O)—OR¹⁷, —OR¹⁸, —O—C(═O)—R¹⁵, —SO₂—R¹⁵, —SO—R¹⁵, —N(R¹⁵)₂—SO₂—R¹⁵, —N(R¹⁵)₂—SO—R¹⁵, or
cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of independently unsubstituted or substituted with one or more R¹⁶,
with the proviso that the aryl or heteroaryl are substituted with no more than one —OR¹⁸ group, and
with the proviso that -L-R¹⁰ is not 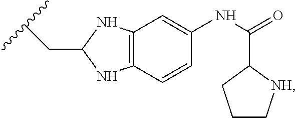 [SMILES: *CC1Nc2ccc(NC(=O)C3CCCN3)cc2N1]
L represents a chain of at most 5 atoms in length,
each R¹¹ independently represents H, or
alkyl, alkenyl, alkynyl, or alkenynyl, each of which independently unsubstituted of substituted with one or more R³⁰,
each R¹² independently represents H, —C≡N, —N(R¹⁵)₂, -T-COOR¹⁴, or
alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰.

14. The compound of claim 13, wherein M represents —C(R⁷)═,

15. The compound of claim 13 or 14, wherein R⁷ represents H or alkyl-N(R⁹)₂, preferably wherein both R⁹ in alkyl-N(R⁹)₂ represent alkyl, preferably methyl.

16. The compound of claim 13 or 14, wherein R⁷ represents H or alkyl (which is preferably methyl), and preferably R⁷ represents H.

17. The compound of any one of claims 13 to 16, wherein the chain in L is at most 4 atoms in length, preferably at most 3 atoms in length.

18. The compound of any one of claims 13 to 17, wherein the chain in L is at least 1 atom in length, preferably at least 2 atoms in length.

19. The compound of any one of claims 13 to 18, wherein the chain in L is 3 atoms in length

20. The compound of any one of claims 13 to 19, wherein L represents:
—C(R¹²)₂—N(R¹¹)—,
—C(R¹²)₂—C(═O)—N(R¹¹)—,
—C(R¹²)₂—N(R¹¹)—C(═O)—,
—C(R¹²)₂—C(R¹²)₂—N(R¹¹)—C(═O)—,
—C(R¹²)₂—C(R¹²)₂—C(═O)—N(R¹¹)—,
—C(R¹²)₂—N(R¹¹)—C(═O)—C(R¹²)₂—,
—C(R¹²)₂—C(═O)—N(R¹¹)—C(R¹²)₂—,
—C(R¹²)₂—C(═O)—N(R¹¹)—C(R¹²)₂—C(R¹²)₂—, or
—C(R¹²)₂—N(R¹¹)—C(═O)—C(R¹²)₂—C(R¹²)₂—,
preferably:
—C(R¹²)₂—N(R¹¹)—,
—C(R¹²)₂—C(═O)—N(R¹¹)—,
—C(R¹²)₂—C(R¹²)₂—N(R¹¹)—C(═O)—,
—C(R¹²)₂—C(═O)—N(R¹¹)—C(R¹²)₂— or
—C(R¹²)₂—C(═O)—N(R¹¹)—C(R¹²)₂—C(R¹²)₂—,
and most preferably —C(R¹²)₂—C(═O)—N(R¹¹)—.

21. The compound of any one of claims 13 to 20, wherein L is free of a —N(R¹¹)— group in which R¹¹ forms a cycle with R¹⁰ or R¹².

22. The compound of any one of claims 13 to 21, being free of a R¹² that represents -T-COOH, preferably -T-COOR¹⁴.

23. The compound of any one of claims 13 to 22, wherein all R¹² represent H.

24. The compound of any one of claims 13 to 22, wherein one R¹² on a carbon atom is H and the other R¹² on the same carbon atom is —C≡N, alkyl or -T-COOR¹⁴, with the proviso that when said other R¹² is -T-COOR¹⁴, R¹⁰ represents an aryl or a heteroaryl.

25. The compound of any one of claims 13 to 22, wherein one R¹² in L is —C≡N or —COOH, and all the others R¹² in L are H, with the proviso that when said one R¹² is —COOH, R¹⁰ represents an aryl or a heteroaryl.

26. The compound of any one of claims 13 to 25, wherein, when R¹⁰ represents an aryl or a heteroaryl, preferably when L is a chain of 5 atoms in length, one R¹² on a first carbon atom in L is —C≡N, one R¹² on a second carbon atom in L is -T-COOR¹⁴, and all others R¹² in L are H.

27. The compound of any one of claims 13 to 26, wherein R¹¹ independently represents H or alkyl, alkenyl, alkynyl, alkenynyl, preferably H or alkyl (which is preferably methyl), and more preferably H.

28. The compound of any one of claims 13 to 27, wherein T is a covalent bond.

29. The compound of any one of claims 13 to 28, wherein R¹⁴ is H.

30. The compound of any one of claims 13 to 29, wherein —C(R¹²)₂—C(═O)—N(R¹¹)— represents —CH₂—C(═O)—N(R¹¹)—, or —CH(CN)—C(═O)—N(R¹¹)—.

31. The compound of any one of claims 13 to 30, wherein:
—C(R¹²)₂—N(R¹¹)— represents —CH₂—NH—,
—C(R¹²)₂—C(═O)—N(R¹¹)— represents —CH₂—C(═O)—NH— or —CH(CN)—C(═O)—NH,
—C(R¹²)₂—C(R¹²)₂—N(R¹¹)—C(═O)— represents —CH₂—CH₂—N(H)—C(═O)—,
—C(R¹²)₂—C(═O)—N(R¹¹)—C(R¹²)₂— represents —CH₂—C(═O)—N(alkyl)-CH(alkyl)- (in which both alkyls are preferably C₁₋₆ alkyl, more preferably methyl), and/or
—C(R¹²)₂—C(═O)—N(R¹¹)—C(R¹²)₂—C(R¹²)₂—
represents —CH₂—C(═O)—NH—CH₂—CH₂— or —CH(C≡N)—C(═O)—NH—CH(—COOH)—CH₂—,
with the proviso that when —C(R¹²)₂—C(═O)—N(R¹¹)—C(R¹²)₂—C(R¹²)₂-represents —CH₂—C(═O)—NH—CH₂—CH₂— or —CH(C≡N)—C(═O)—NH—CH(—COOH)—CH₂—, R¹⁰ represents an aryl or a heteroaryl.

32. The compound of any one of claims 13 to 31, wherein R¹⁰ represents:
a 6-membered cycle selected from heterocycloalkyl, heterocycloalkenyl, heterocycloalkynyl, or heterocycloalkenynyl, each of which comprising one or more nitrogen ring atoms as sole heteroatoms, and each of which being independently unsubstituted or substituted as described in claim 1,
or
aryl or heteroaryl, each of which independently unsubstituted or substituted as described in claim 1, preferably
a 6-membered cycle that is a heterocycloalkyl comprising one or more nitrogen ring atoms as sole heteroatoms, being unsubstituted or substituted as described in claim 1,
or
aryl or heteroaryl, each of which independently unsubstituted or substituted as described in claim 1.

33. The compound of any one of claims 13 to 32, wherein R¹⁰ represents a heterocycloalkyl, preferably piperidinyl (preferably piperidin-4-yl) or piperazinyl (preferably piperazin-1-yl), each of which independently unsubstituted or substituted as described in claim 1.

34. The compound of any one of claims 13 to 33, wherein, when R¹⁰ represents a 6-membered cycle, the 6-membered cycle is unsubstituted or substituted with alkyl, preferably C₁₋₆ alkyl, more preferably C₁₋₃ alkyl, preferably methyl, ethyl or isopropryl.

35. The compound of any one of claims 13 to 32, wherein R¹⁰ represents an aryl, preferably phenyl, unsubstituted or substituted as described in claim 1.

36. The compound of any one of claims 13 to 32 and 35, wherein the aryl is unsubstituted or substituted with one or more, preferably one, of:
—N(R¹⁵)—C(═O)—R¹⁵, preferably —NH—C(═O)—R¹⁵, preferably wherein R¹⁵ is alkyl (preferably methyl) unsubstituted or substituted with one or more (preferably one)
amino, alkylamino, dialkylamino, preferably dialkylamino, preferably wherein the alkyl is C₁₋₆ alkyl, preferably C₁₋₃ alkyl, preferably methyl,
—OR¹⁸, preferably wherein R¹⁸ is H or alkyl unsubstituted or substituted with one or more (preferably one) amino, alkylamino, dialkylamino, preferably dialkylamino, preferably wherein the alkyl is C₁₋₆ alkyl, preferably C₁₋₃ alkyl, preferably methyl, or
alkyl-N(R¹⁵)₂, preferably wherein the alkyl is C₁₋₆ alkyl, preferably C₁₋₃ alkyl, preferably methyl;
preferably wherein R¹⁵ is alkyl, preferably C₁₋₆ alkyl, preferably C₁₋₃ alkyl, preferably methyl.

37. The compound of any one of claims 13 to 32, wherein R¹⁰ represents a heteroaryl, preferably pyridinyl (preferably pyridin-3-yl or pyridin-4-yl) or pyrimidinyl (preferably pyrimidin-2-yl), each of which independently unsubstituted or substituted as described in claim 1.

38. The compound of any one of claims 13 to 32 and 37, wherein the heteroaryl is unsubstituted.

39. The compound of claim 1, being: 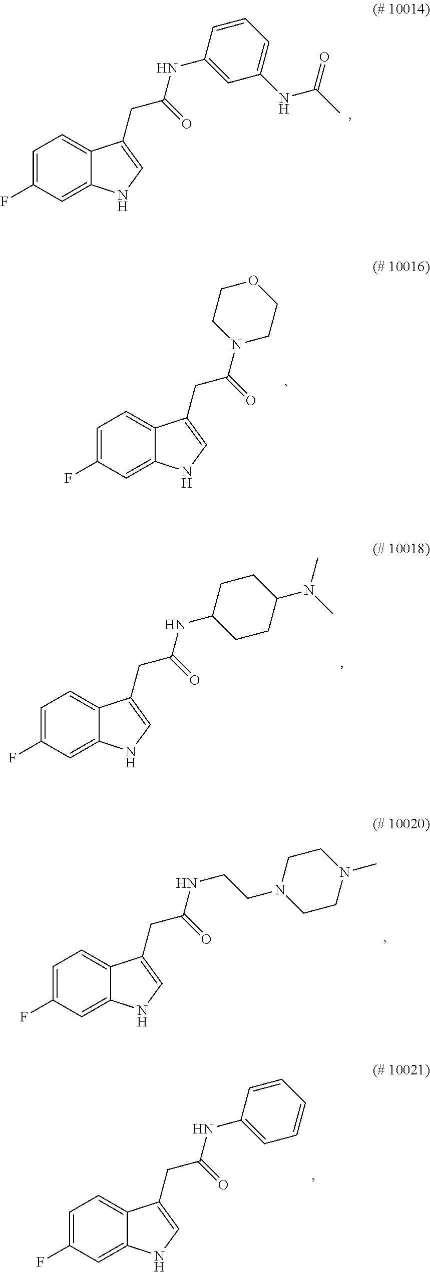 [SMILES: CC(=O)Nc1cccc(NC(=O)Cc2c[nH]c3cc(F)ccc23)c1 ; O=C(Cc1c[nH]c2cc(F)ccc12)N1CCOCC1 ; CN(C)C1CCC(NC(=O)Cc2c[nH]c3cc(F)ccc23)CC1 ; CN1CCN(CCNC(=O)Cc2c[nH]c3cc(F)ccc23)CC1 ; O=C(Cc1c[nH]c2cc(F)ccc12)Nc1ccccc1] 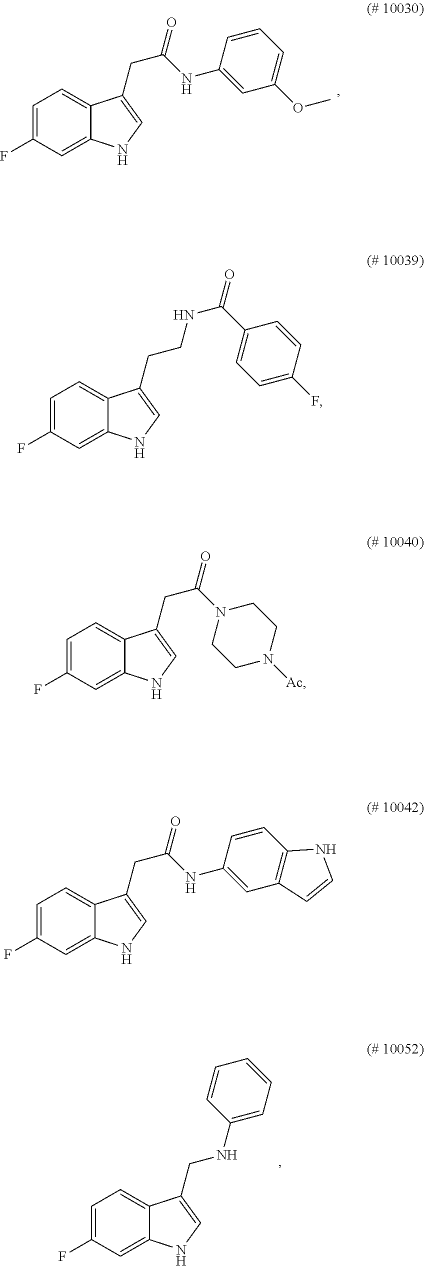 [SMILES: COc1cccc(NC(=O)Cc2c[nH]c3cc(F)ccc23)c1 ; O=C(NCCc1c[nH]c2cc(F)ccc12)c1ccc(F)cc1 ; CC(=O)N1CCN(C(=O)Cc2c[nH]c3cc(F)ccc23)CC1 ; O=C(Cc1c[nH]c2cc(F)ccc12)Nc1ccc2[nH]ccc2c1 ; Fc1ccc2c(CNc3ccccc3)c[nH]c2c1] 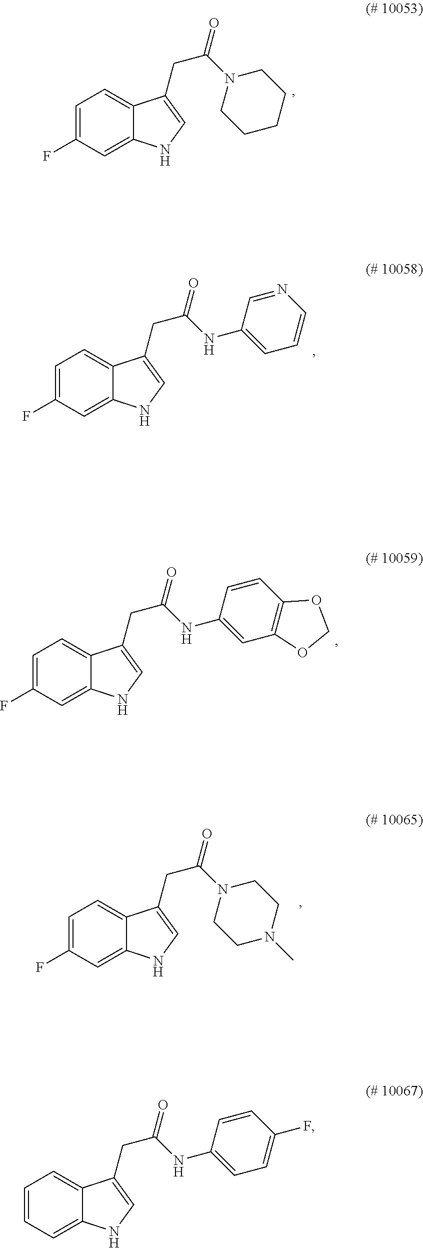 [SMILES: O=C(Cc1c[nH]c2cc(F)ccc12)N1CCCCC1 ; O=C(Cc1c[nH]c2cc(F)ccc12)Nc1cccnc1 ; O=C(Cc1c[nH]c2cc(F)ccc12)Nc1ccc2c(c1)OCO2 ; CN1CCN(C(=O)Cc2c[nH]c3cc(F)ccc23)CC1 ; O=C(Cc1c[nH]c2ccccc12)Nc1ccc(F)cc1] 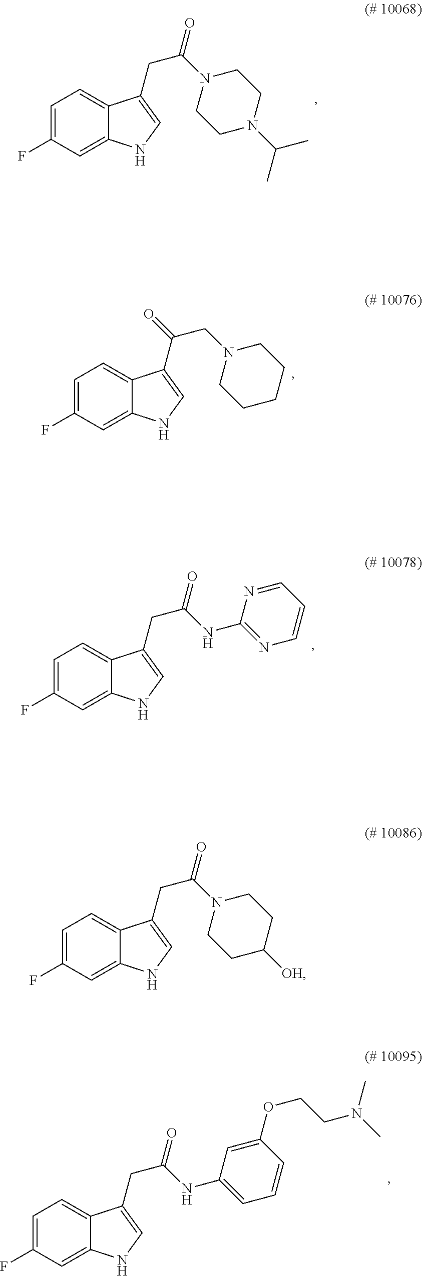 [SMILES: CC(C)N1CCN(C(=O)Cc2c[nH]c3cc(F)ccc23)CC1 ; O=C(CN1CCCCC1)c1c[nH]c2cc(F)ccc12 ; O=C(Cc1c[nH]c2cc(F)ccc12)Nc1ncccn1 ; O=C(Cc1c[nH]c2cc(F)ccc12)N1CCC(O)CC1 ; CN(C)CCOc1cccc(NC(=O)Cc2c[nH]c3cc(F)ccc23)c1] 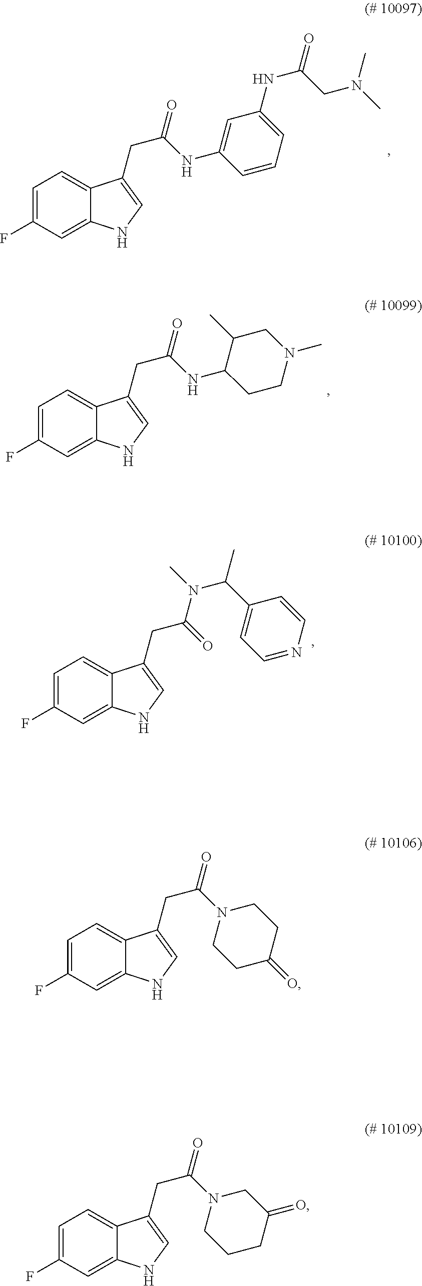 [SMILES: CN(C)CC(=O)Nc1cccc(NC(=O)Cc2c[nH]c3cc(F)ccc23)c1 ; CC1CN(C)CCC1NC(=O)Cc1c[nH]c2cc(F)ccc12 ; CC(c1ccncc1)N(C)C(=O)Cc1c[nH]c2cc(F)ccc12 ; O=C1CCN(C(=O)Cc2c[nH]c3cc(F)ccc23)CC1 ; O=C1CCCN(C(=O)Cc2c[nH]c3cc(F)ccc23)C1] 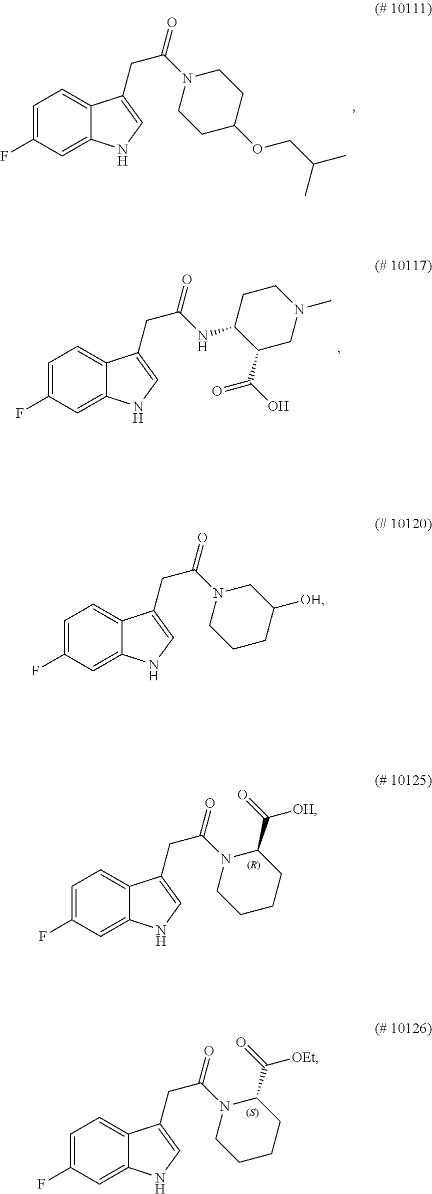 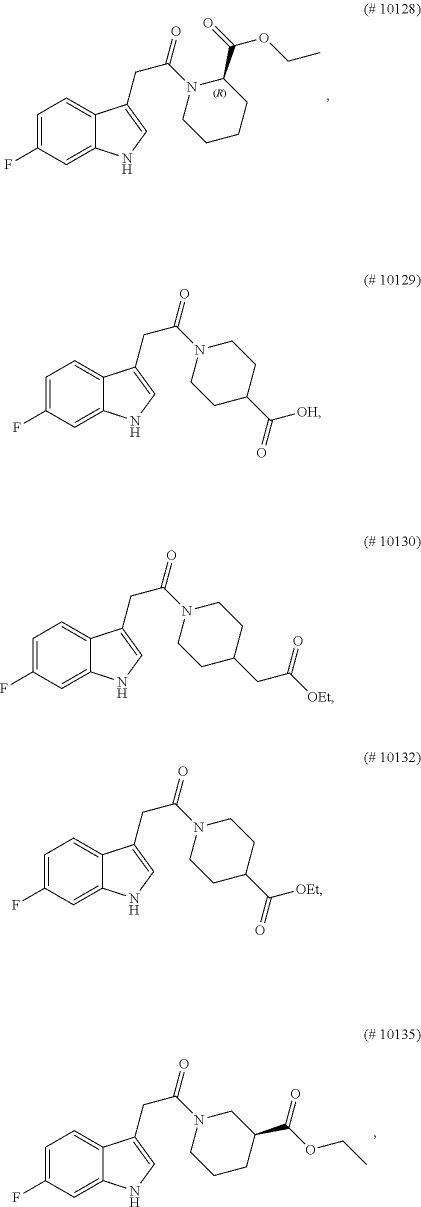 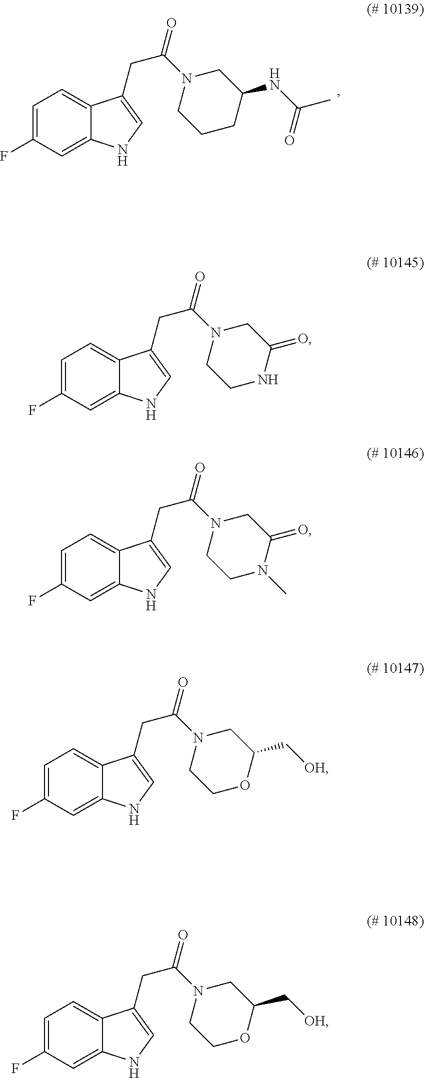 [SMILES: CC(=O)N[C@H]1CCCN(C(=O)Cc2c[nH]c3cc(F)ccc23)C1 ; O=C1CN(C(=O)Cc2c[nH]c3cc(F)ccc23)CCN1 ; CN1CCN(C(=O)Cc2c[nH]c3cc(F)ccc23)CC1=O ; O=C(Cc1c[nH]c2cc(F)ccc12)N1CCO[C@@H](CO)C1 ; O=C(Cc1c[nH]c2cc(F)ccc12)N1CCO[C@H](CO)C1] 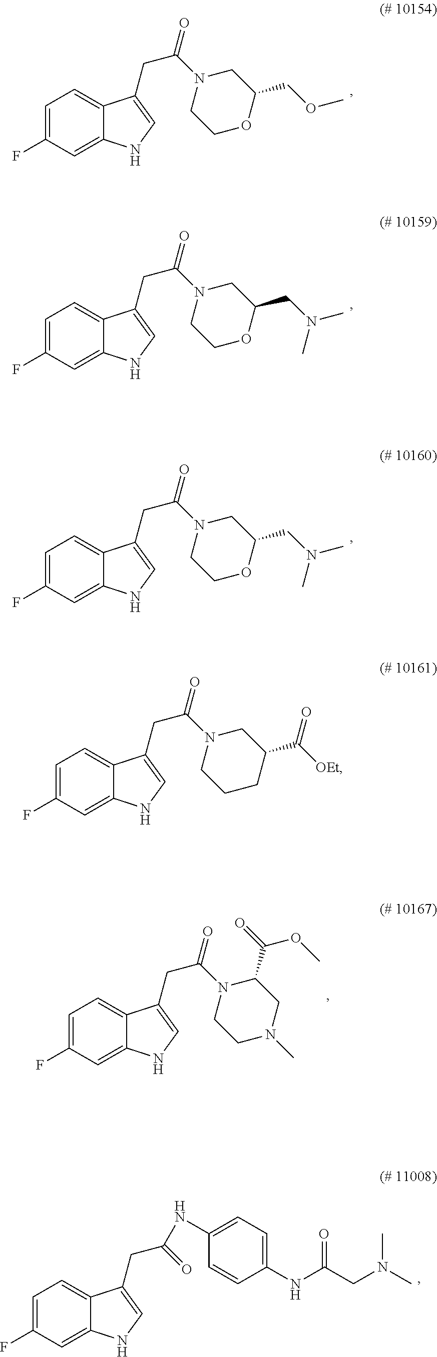 [SMILES: COC[C@H]1CN(C(=O)Cc2c[nH]c3cc(F)ccc23)CCO1 ; CN(C)C[C@@H]1CN(C(=O)Cc2c[nH]c3cc(F)ccc23)CCO1 ; CN(C)C[C@H]1CN(C(=O)Cc2c[nH]c3cc(F)ccc23)CCO1 ; CCOC(=O)[C@@H]1CCCN(C(=O)Cc2c[nH]c3cc(F)ccc23)C1 ; COC(=O)[C@@H]1CN(C)CCN1C(=O)Cc1c[nH]c2cc(F)ccc12 ; CN(C)CC(=O)Nc1ccc(NC(=O)Cc2c[nH]c3cc(F)ccc23)cc1] 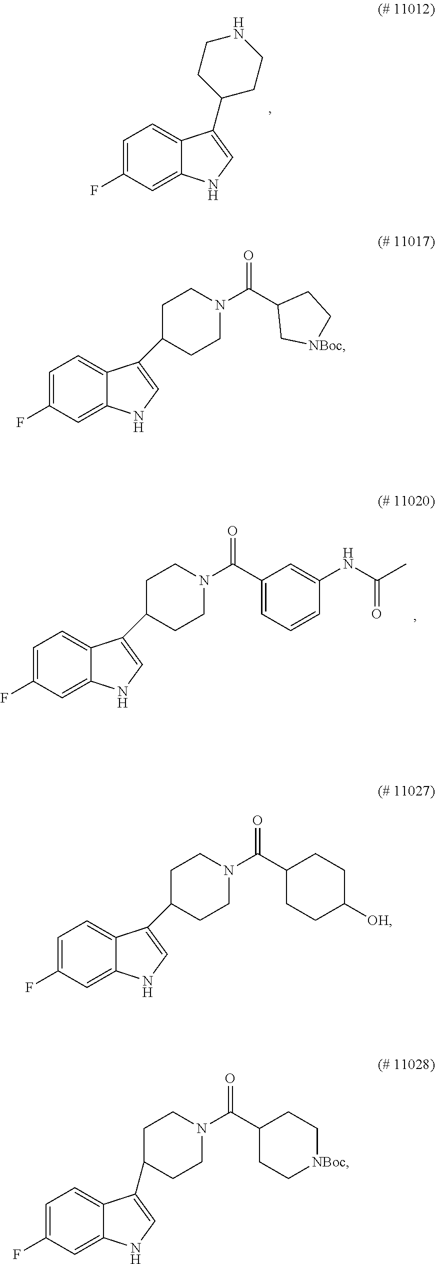 [SMILES: Fc1ccc2c(C3CCNCC3)c[nH]c2c1 ; CC(C)(C)OC(=O)N1CCC(C(=O)N2CCC(c3c[nH]c4cc(F)ccc34)CC2)C1 ; CC(=O)Nc1cccc(C(=O)N2CCC(c3c[nH]c4cc(F)ccc34)CC2)c1 ; O=C(C1CCC(O)CC1)N1CCC(c2c[nH]c3cc(F)ccc23)CC1 ; CC(C)(C)OC(=O)N1CCC(C(=O)N2CCC(c3c[nH]c4cc(F)ccc34)CC2)CC1] 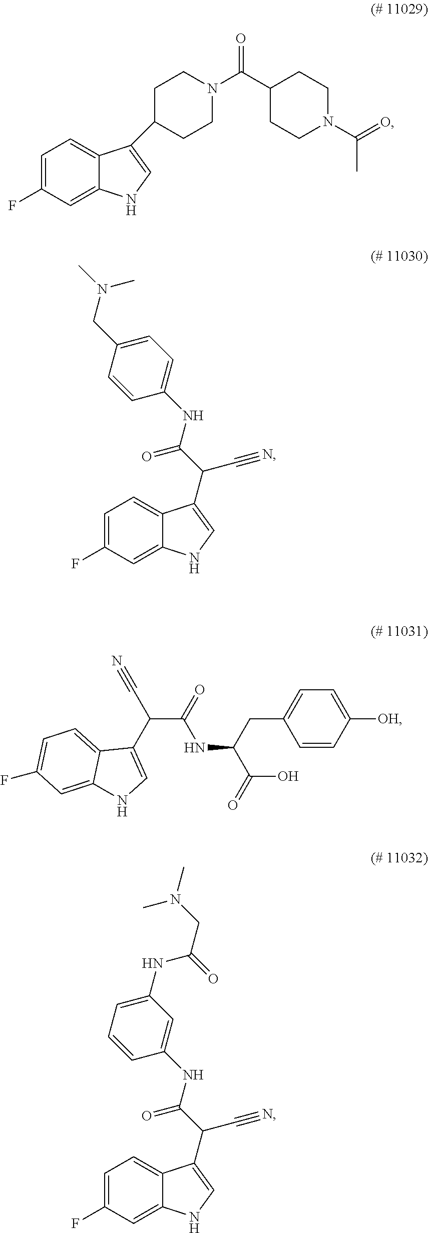 [SMILES: CC(=O)N1CCC(C(=O)N2CCC(c3c[nH]c4cc(F)ccc34)CC2)CC1 ; CN(C)Cc1ccc(NC(=O)C(C#N)c2c[nH]c3cc(F)ccc23)cc1 ; N#CC(C(=O)N[C@@H](Cc1ccc(O)cc1)C(=O)O)c1c[nH]c2cc(F)ccc12 ; CN(C)CC(=O)Nc1cccc(NC(=O)C(C#N)c2c[nH]c3cc(F)ccc23)c1] 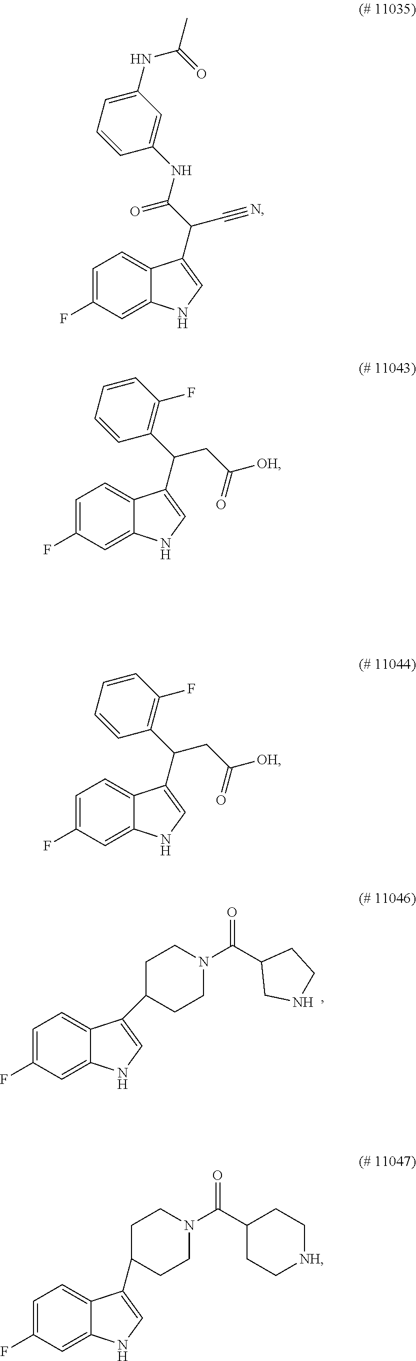 [SMILES: CC(=O)Nc1cccc(NC(=O)C(C#N)c2c[nH]c3cc(F)ccc23)c1 ; O=C(O)CC(c1ccccc1F)c1c[nH]c2cc(F)ccc12 ; O=C(O)CC(c1ccccc1F)c1c[nH]c2cc(F)ccc12 ; O=C(C1CCNC1)N1CCC(c2c[nH]c3cc(F)ccc23)CC1 ; O=C(C1CCNCC1)N1CCC(c2c[nH]c3cc(F)ccc23)CC1] 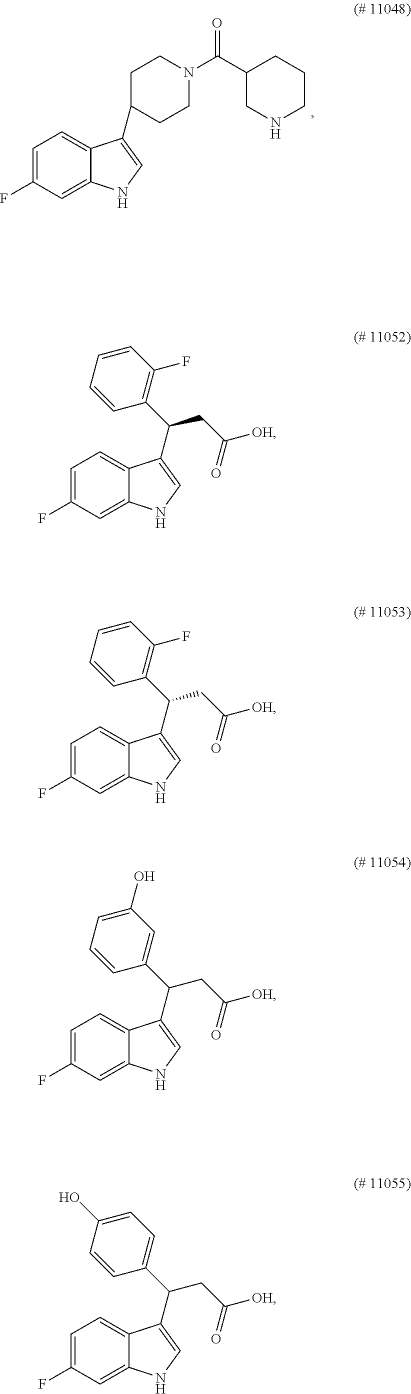 [SMILES: O=C(C1CCCNC1)N1CCC(c2c[nH]c3cc(F)ccc23)CC1 ; O=C(O)C[C@H](c1ccccc1F)c1c[nH]c2cc(F)ccc12 ; O=C(O)C[C@@H](c1ccccc1F)c1c[nH]c2cc(F)ccc12 ; O=C(O)CC(c1cccc(O)c1)c1c[nH]c2cc(F)ccc12 ; O=C(O)CC(c1ccc(O)cc1)c1c[nH]c2cc(F)ccc12] 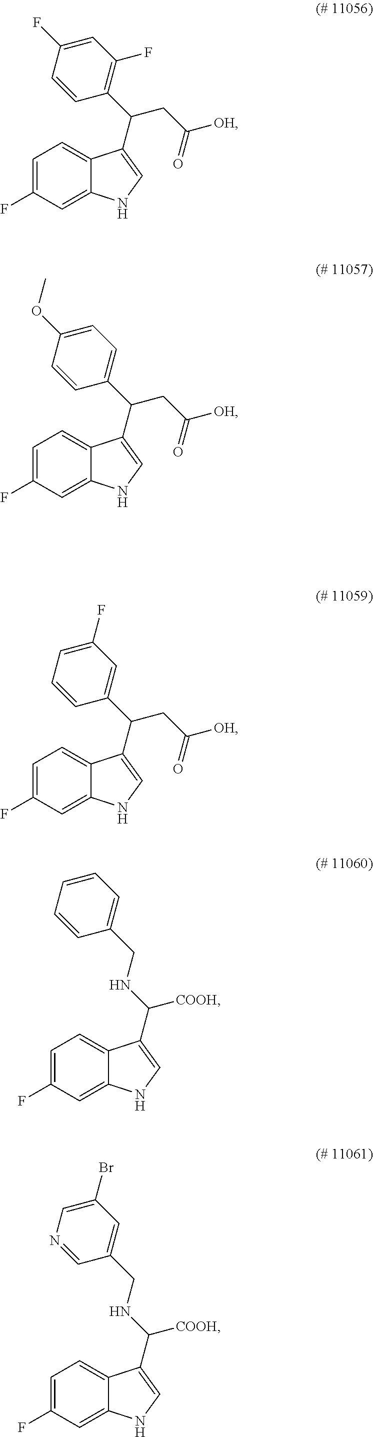 [SMILES: O=C(O)CC(c1ccc(F)cc1F)c1c[nH]c2cc(F)ccc12 ; COc1ccc(C(CC(=O)O)c2c[nH]c3cc(F)ccc23)cc1 ; O=C(O)CC(c1cccc(F)c1)c1c[nH]c2cc(F)ccc12 ; O=C(O)C(NCc1ccccc1)c1c[nH]c2cc(F)ccc12 ; O=C(O)C(NCc1cncc(Br)c1)c1c[nH]c2cc(F)ccc12] 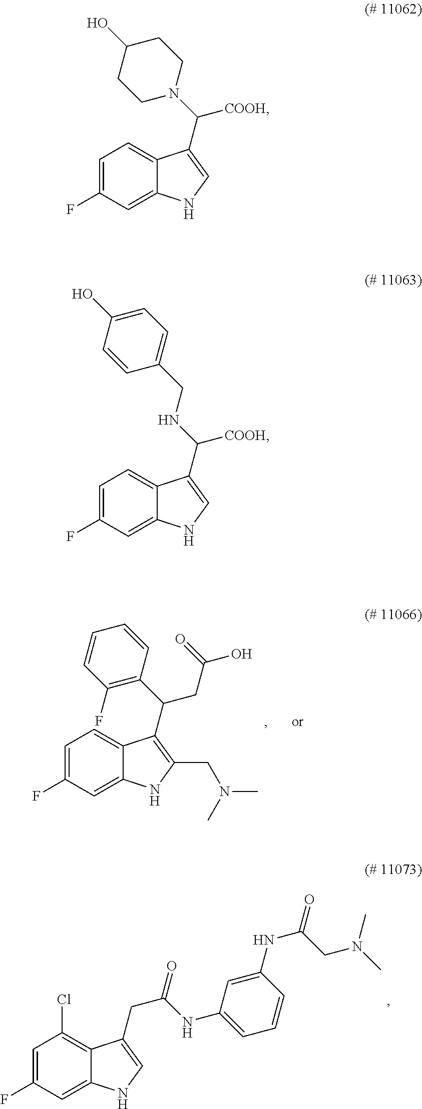 [SMILES: O=C(O)C(c1c[nH]c2cc(F)ccc12)N1CCC(O)CC1 ; O=C(O)C(NCc1ccc(O)cc1)c1c[nH]c2cc(F)ccc12 ; CN(C)Cc1[nH]c2cc(F)ccc2c1C(CC(=O)O)c1ccccc1F ; CN(C)CC(=O)Nc1cccc(NC(=O)Cc2c[nH]c3cc(F)cc(Cl)c23)c1]
or a pharmaceutically acceptable salt, ester, solvate, isomer, or tautomer thereof.

40. The compound of claim 1 being Compound #10014, 10018, 10040, 10053, 10076, 10086, 10095, 10097, 10159, 11008, 11032, 11043, 11052, 11053, 11054, 11055, 11066, or 11073,

41. The compound of claim 1 being Compound #10014, 10095, 10097, 11008, 11043, 11052, 11053, 11054, 11055, 11066, or 11073.

42. The compound of claim 1 being Compound #10018, 10040, 10086, 10095, 10097, 10032, 11053, or 11066.

43. The compound of claim 1 being Compound #10095, 10097, 11032, 11053, or 11073.

44. The compound of claim 1 being Compound #11032, 11095, or 11097.

45. The compound of claim 1 being Compound #10018, 10040, 10086, or 11066.

46. The compound of any one of claims 1 to 12, wherein B)
R¹⁰ represents —COOH,
L represents a chain that is at most 3 atoms in length, and that is not —CH₂—CH(NH₂)—,
any R¹¹ independently represents H, or
alkyl, alkenyl, alkynyl, or alkenynyl, each of which independently unsubstituted or substituted with one or more R³⁰, and
any R¹² independently represents H, —C≡N, —N(R¹⁵)₂, —C(═O)—R¹⁵, or
alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰,

47. The compound of claim 46, wherein G represents —C(R⁴)═, preferably wherein R⁴ represents a halogen atom, preferably F.

48. The compound of claim 46 or 47, wherein L represents a chain that is at most 2 atoms in length, preferably only 1 atom in length.

49. The compound of any one of claims 46 to 48, wherein L represents —C(R¹²)₂— or —C(R¹²)₂—C(R¹²)₂—.

50. The compound of any one of claims 46 to 49, wherein all R¹² represent H.

51. The compound of claim any one of claims 46 to 49, wherein at least one (preferably one) R¹² represents:
—C≡N, —N(R¹⁵)₂, —C(═O)—R¹⁵, or
alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰,
preferably —N(R¹⁵)₂, —C(═O)—R¹⁵, or
cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³⁰,
more preferably —N(R¹⁵)₂, —C(═O)—R¹⁵, or
heterocycloalkyl, heterocycloalkenyl, heterocycloalkynyl, heterocycloalkenynyl, aryl, or heteroaryl, each of which independently unsubstituted or substituted with one or more R³⁰, or
yet more preferably —N(R¹⁵)₂, —C(═O)—R¹⁵, or
heterocycloalkyl or aryl, each of which independently unsubstituted or substituted with one or more R³⁰,
and preferably any other R¹² represent H.

52. The compound of any one of claims 46 to 51, wherein each R³⁰ independently is a halogen atom, —OR¹⁵, or alkyl, alkenyl, alkynyl, alkenynyl, each of which independently unsubstituted or substituted with one or more R¹⁶, preferably unsubstituted,
preferably a halogen atom, —OR¹⁵, or alkyl unsubstituted or substituted with one or more R¹⁶, preferably unsubstituted.

53. The compound of any one of claims 46 to 52, wherein R¹⁵ represents H or alkyl, alkenyl, alkynyl, or alkenynyl, each of which independently unsubstituted or substituted with one or more R¹⁶, preferably unsubstituted.

54. The compound of any one of claims 46 to 53, wherein R¹⁵ represents H or alkyl unsubstituted or substituted with one or more R¹⁶, preferably unsubstituted.

55. The compound of claim 1 being 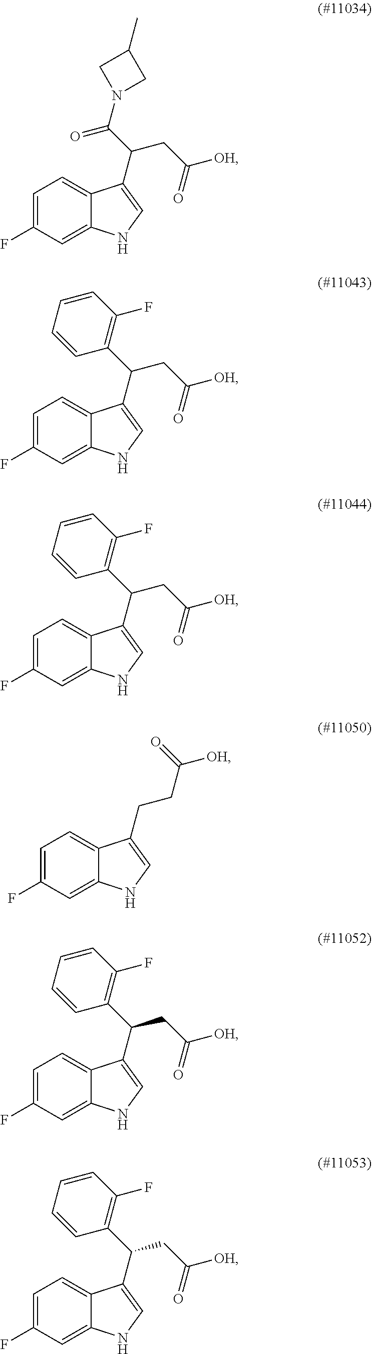 [SMILES: CC1CN(C(=O)C(CC(=O)O)c2c[nH]c3cc(F)ccc23)C1 ; O=C(O)CC(c1ccccc1F)c1c[nH]c2cc(F)ccc12 ; O=C(O)CC(c1ccccc1F)c1c[nH]c2cc(F)ccc12 ; O=C(O)CCc1c[nH]c2cc(F)ccc12 ; O=C(O)C[C@H](c1ccccc1F)c1c[nH]c2cc(F)ccc12 ; O=C(O)C[C@@H](c1ccccc1F)c1c[nH]c2cc(F)ccc12] 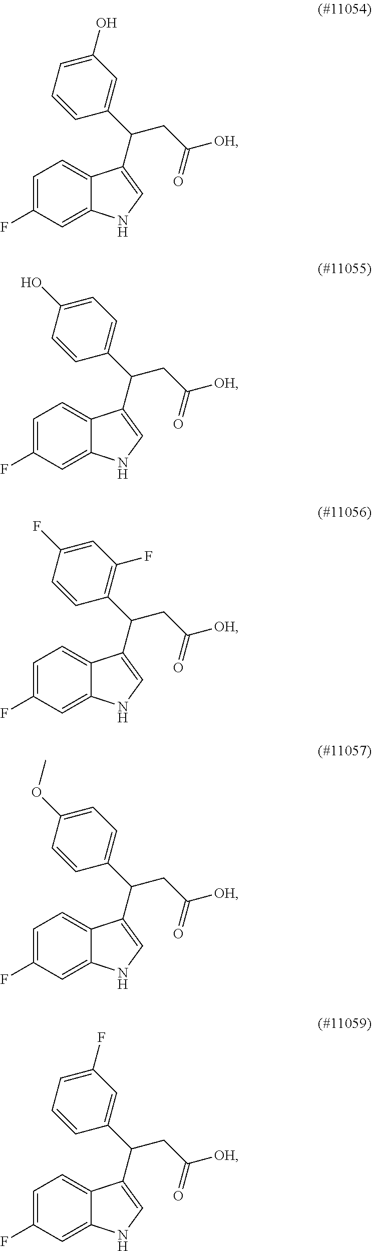 [SMILES: O=C(O)CC(c1cccc(O)c1)c1c[nH]c2cc(F)ccc12 ; O=C(O)CC(c1ccc(O)cc1)c1c[nH]c2cc(F)ccc12 ; O=C(O)CC(c1ccc(F)cc1F)c1c[nH]c2cc(F)ccc12 ; COc1ccc(C(CC(=O)O)c2c[nH]c3cc(F)ccc23)cc1 ; O=C(O)CC(c1cccc(F)c1)c1c[nH]c2cc(F)ccc12] 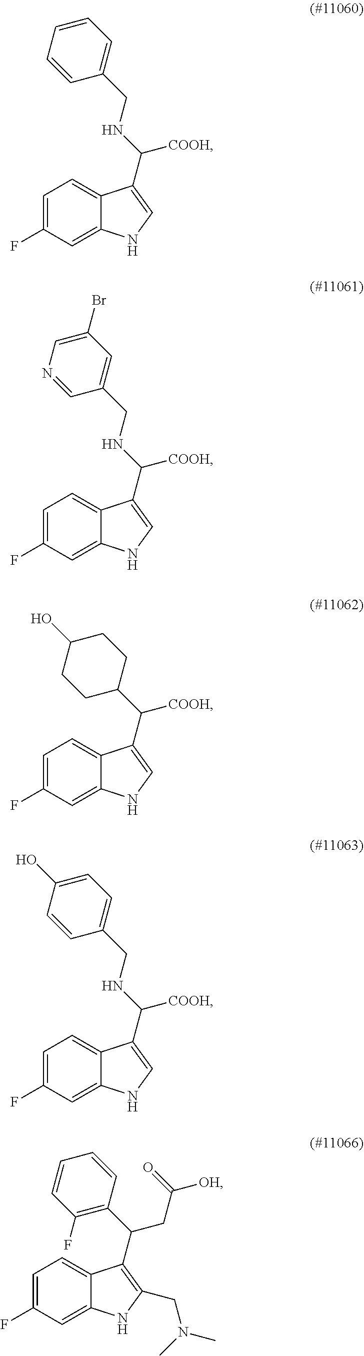 [SMILES: O=C(O)C(NCc1ccccc1)c1c[nH]c2cc(F)ccc12 ; O=C(O)C(NCc1cncc(Br)c1)c1c[nH]c2cc(F)ccc12 ; O=C(O)C(c1c[nH]c2cc(F)ccc12)C1CCC(O)CC1 ; O=C(O)C(NCc1ccc(O)cc1)c1c[nH]c2cc(F)ccc12 ; CN(C)Cc1[nH]c2cc(F)ccc2c1C(CC(=O)O)c1ccccc1F]
or a pharmaceutically acceptable salt, ester, solvate, isomer, or tautomer thereof.

56. The compound of claim 1, being compound #11066.

57. The compound of any one of claims 1 to 12, wherein C)
R¹⁰ is attached to a nitrogen atom of a —N(R¹¹)— group that ends the chain in L, and R¹⁰ and the R¹¹ of the —N(R¹¹)— group that ends the chain in L together with the nitrogen atom to which they are attached form a heteroaryl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl, each of which independently unsubstituted or substituted with:
a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR²⁰, —OR¹⁵, —O—C(═O)—R¹⁵, —SO₂—R¹⁵, —SO—R¹⁵, —N(R¹⁵)₂—SO₂—R¹⁵, —N(R¹⁵)₂—SO—R¹⁵, or
alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R²¹,
with the proviso that when R¹⁰ and R¹¹ together with the nitrogen atom to which they are attached form morpholinyl, the morpholinyl is free of a substituent containing a —C(═O)— group,
with the proviso that when R¹⁰ and R¹¹ together with the nitrogen atom to which they are attached form piperazinyl substituted with a —C(═O)—O—CH₃ group, the —C(═O)—O—CH₃ group is in position 2,
with the proviso that when R¹⁰ and R¹¹ together with the nitrogen atom to which they are attached form piperazinyl N-substituted with —O-heterocycloalkyl, the heterocycloalkyl is other than oxalanyl,
the chain in L is at most 4 atoms in length and ends with the —N(R¹¹)— group to which R¹⁰ is attached,
with the proviso that -L-R¹⁰ is not 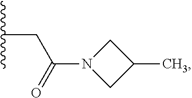 [SMILES: *CC(=O)N1CC(C)C1]
any other R¹¹ independently represents H, or
alkyl, alkenyl, alkynyl, or alkenynyl, each of which independently unsubstituted or substituted with one or more R³¹ and
any R¹² independently represents H, —C≡N, —N(R¹⁵)₂, or
alkyl, alkenyl, alkynyl, alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, eterocycloalkenynonyl, aryl, or heteroaryl, each of which independently unsubstituted or substituted with one or more R³¹,
with the proviso that when one R¹² on a given carbon atom is methyl, any other R¹² on said given carbon atom is other than methyl.

58. The compound of claim 57, wherein L comprises at least one —C(═O)— and a least one-N(R¹¹)—.

59. The compound of claim 57 or 58, wherein L comprises exactly one —C(═O)— and exactly one-N(R¹¹)—.

60. The compound of any one of claims 57 to 59, wherein -L- ends with a —C(═O)—N(R¹¹)— group.

61. The compound of any one of claims 57 to 60, wherein L represents —C(R¹²)₂—C(═O)—N(R¹¹)— or —C(═O)—C(R¹²)₂—N(R¹¹)—, preferably —C(R¹²)₂—C(═O)—N(R¹¹)—.

62. The compound of any one of claims 57 to 61, wherein each R¹² independently represents H, —C≡N, or alkyl (preferably methyl or ethyl).

63. The compound of any one of claims 57 to 62, wherein a first R¹² on a carbon atom represents alkyl (preferably methyl or ethyl), or —C≡N, and a second R¹² on said carbon atom represent H.

64. The compound of any one of claims 57 to 62, wherein two R¹² on a carbon atom represent alkyl.

65. The compound of any one of claims 57 to 62, wherein two R¹² on a carbon atom represents H.

66. The compound of any one of claims 57 to 62, wherein any other R¹¹ represents H.

67. The compound of any one of claims 57 to 66, wherein R¹⁰ and the R¹¹ together with the nitrogen atom to which they are attached form a heterocycloalkyl, preferably azetidin-1-yl, pyrrolidi-1-nyl, piperidin-1-yl, piperidinon-1-yl (preferably piperidin-3-on-1-yl or piperidin-4-on-1-yl), morpholin-1-yl, piperazin-1-yl, piperazinone-1-yl (more preferably piperazin-3-on-1-yl), 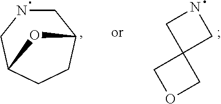
most preferably azetidin-1-yl, piperidin-1-yl, or morpholin-1-yl.

68. The compound of any one of claims 57 to 67, wherein the heteroaryl, heterocycloalkyl, heterocycloalkenyl, heterocycloalkynyl and heterocycloalkenynyl formed by R¹⁰ and R¹¹ together with the nitrogen atom to which they are attached is unsubstituted or substituted with one or more (preferably one or two, more preferably one) of alkyl, —OR¹⁵, —C(═O)—N(R¹⁵)₂, —N(R¹⁵)—C(═O)—R¹⁵, or —C(═O)—OR²⁰,
more preferably unsubstituted or substituted with one or more (preferably one) of:
alkyl (preferably C₁₋₆, more preferably methyl or ethyl), OH,
alkoxy (preferably C₁₋₆, more preferably methoxy, ethoxy, propoxy (preferably isopropoxy), or butoxy (preferably isobutoxy)),
—CONH₂,
═NH—C(═O)-alkyl (preferably C₁₋₆, more preferably ═NH—C(═O)—CH₃),
—C(═O)O-alkyl (preferably C₁₋₆, more preferably —C(═O)O—CH₂CH₃),
and most preferably unsubstituted or substituted with one or more, preferably one or two, more preferably one of alkyl, hydroxyl, or —CH₂—N(CH₃)₂.

69. The compound of any one of claims 57 to 68, wherein the R¹⁵ of the OR¹⁵ group, the alkoxy and/or the alkyl substituting the cycle formed by R¹⁰ and R¹¹ are unsubstituted or substituted with one or more of alkoxy (preferably C₁₋₆, more preferably methoxy), alkoxycarbonyl (preferably C₁₋₆, more preferably —C(═O)OCH₂CH₃), hydroxyl, amino, alkylamino, or dialkylamino (preferably C₁₋₆, more preferably —N(CH₃)₂).

70. The compound of claim 1 being: 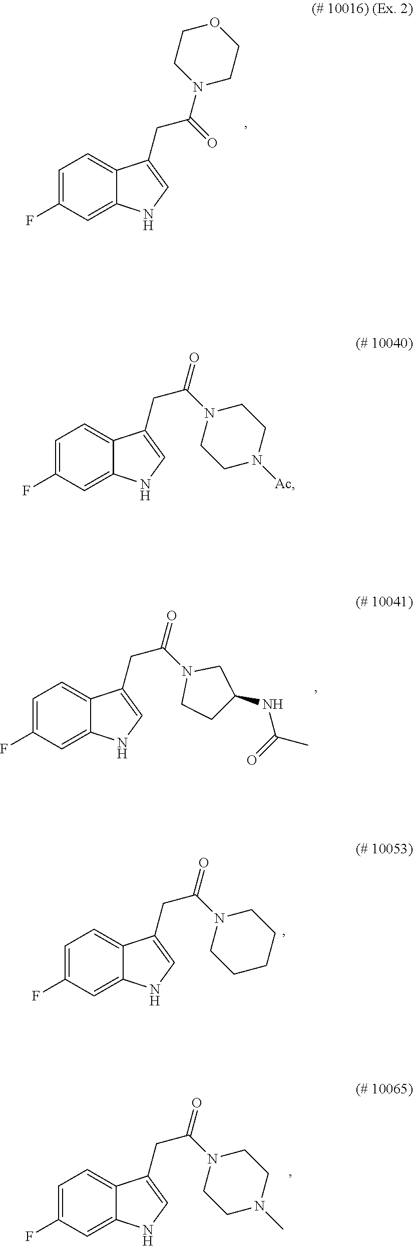 [SMILES: O=C(Cc1c[nH]c2cc(F)ccc12)N1CCOCC1 ; CC(=O)N1CCN(C(=O)Cc2c[nH]c3cc(F)ccc23)CC1 ; CC(=O)N[C@H]1CCN(C(=O)Cc2c[nH]c3cc(F)ccc23)C1 ; O=C(Cc1c[nH]c2cc(F)ccc12)N1CCCCC1 ; CN1CCN(C(=O)Cc2c[nH]c3cc(F)ccc23)CC1] 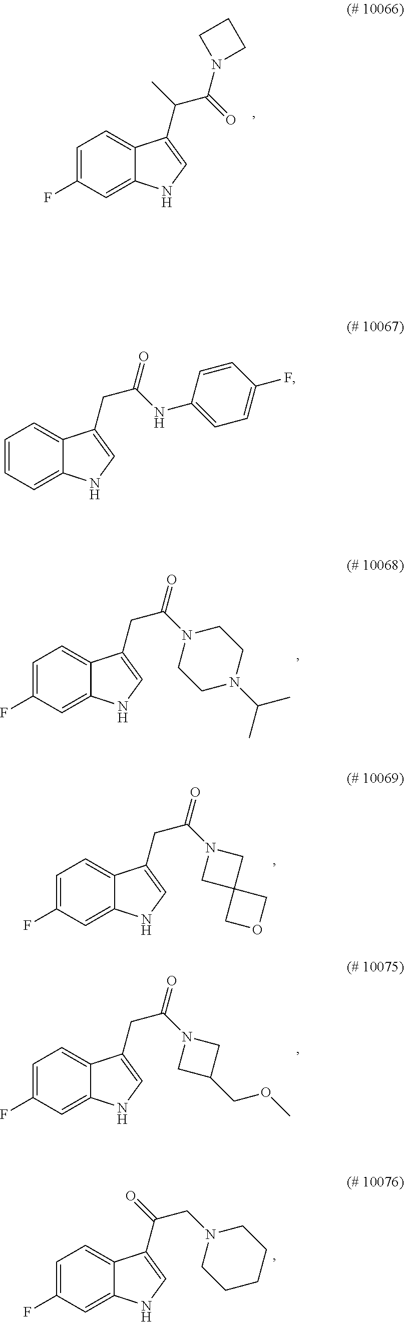 [SMILES: CC(C(=O)N1CCC1)c1c[nH]c2cc(F)ccc12 ; O=C(Cc1c[nH]c2ccccc12)Nc1ccc(F)cc1 ; CC(C)N1CCN(C(=O)Cc2c[nH]c3cc(F)ccc23)CC1 ; O=C(Cc1c[nH]c2cc(F)ccc12)N1CC2(COC2)C1 ; COCC1CN(C(=O)Cc2c[nH]c3cc(F)ccc23)C1 ; O=C(CN1CCCCC1)c1c[nH]c2cc(F)ccc12] 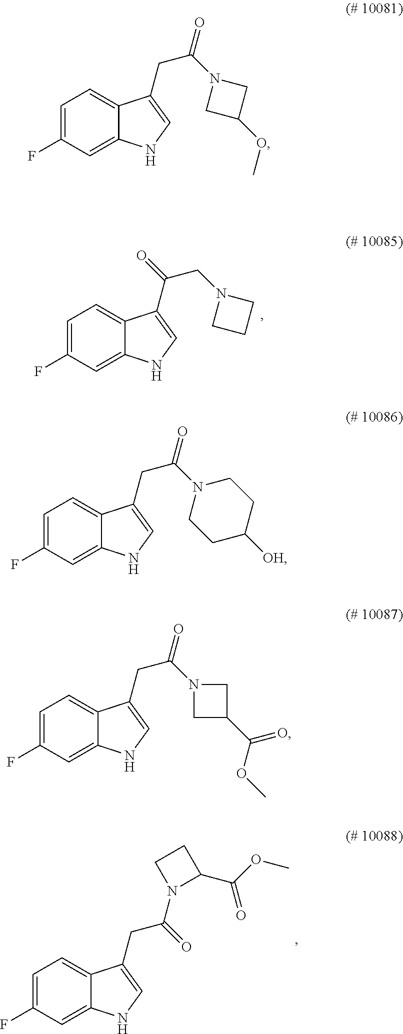 [SMILES: COC1CN(C(=O)Cc2c[nH]c3cc(F)ccc23)C1 ; O=C(CN1CCC1)c1c[nH]c2cc(F)ccc12 ; O=C(Cc1c[nH]c2cc(F)ccc12)N1CCC(O)CC1 ; COC(=O)C1CN(C(=O)Cc2c[nH]c3cc(F)ccc23)C1 ; COC(=O)C1CCN1C(=O)Cc1c[nH]c2cc(F)ccc12] 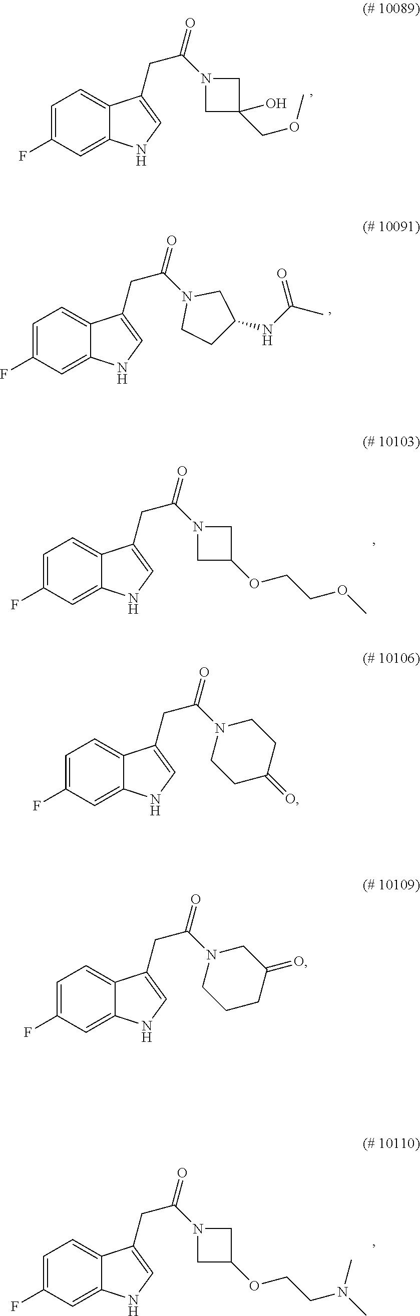 [SMILES: COCC1(O)CN(C(=O)Cc2c[nH]c3cc(F)ccc23)C1 ; CC(=O)N[C@@H]1CCN(C(=O)Cc2c[nH]c3cc(F)ccc23)C1 ; COCCOC1CN(C(=O)Cc2c[nH]c3cc(F)ccc23)C1 ; O=C1CCN(C(=O)Cc2c[nH]c3cc(F)ccc23)CC1 ; O=C1CCCN(C(=O)Cc2c[nH]c3cc(F)ccc23)C1 ; CN(C)CCOC1CN(C(=O)Cc2c[nH]c3cc(F)ccc23)C1] 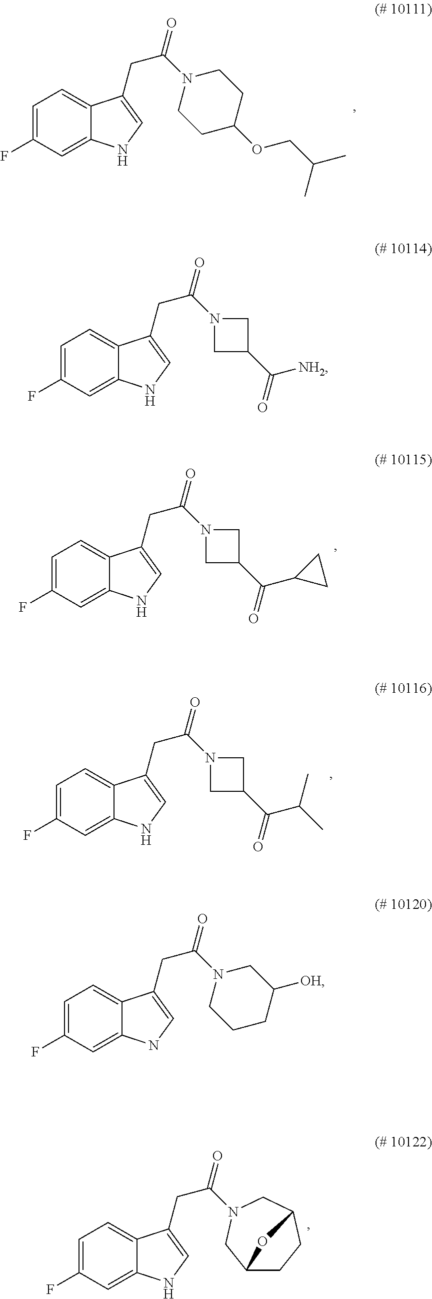 [SMILES: CC(C)COC1CCN(C(=O)Cc2c[nH]c3cc(F)ccc23)CC1 ; NC(=O)C1CN(C(=O)Cc2c[nH]c3cc(F)ccc23)C1 ; O=C(C1CC1)C1CN(C(=O)Cc2c[nH]c3cc(F)ccc23)C1 ; CC(C)C(=O)C1CN(C(=O)Cc2c[nH]c3cc(F)ccc23)C1 ; O=C(Cc1c[nH]c2cc(F)ccc12)N1CCCC(O)C1 ; O=C(Cc1c[nH]c2cc(F)ccc12)N1C[C@H]2CC[C@@H](C1)O2] 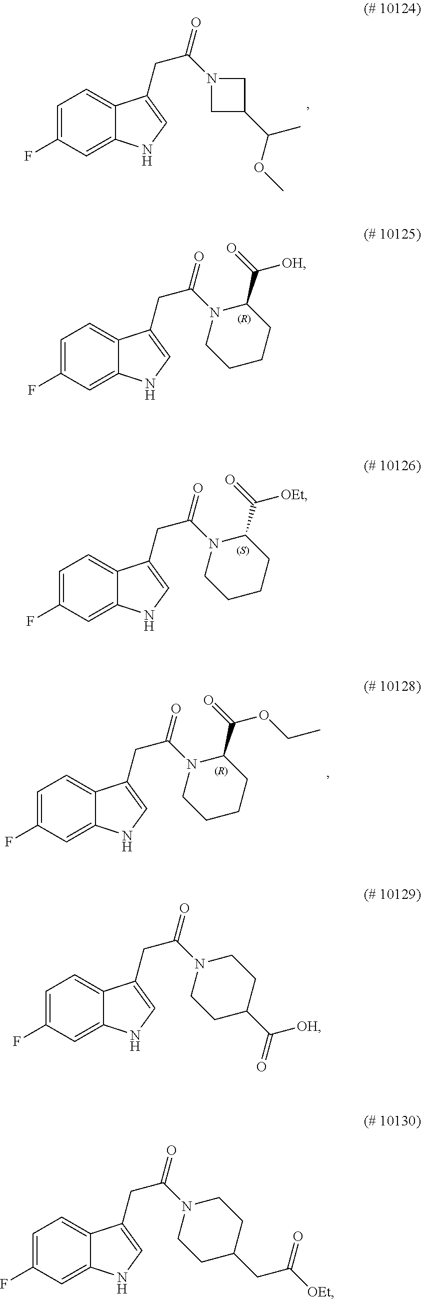 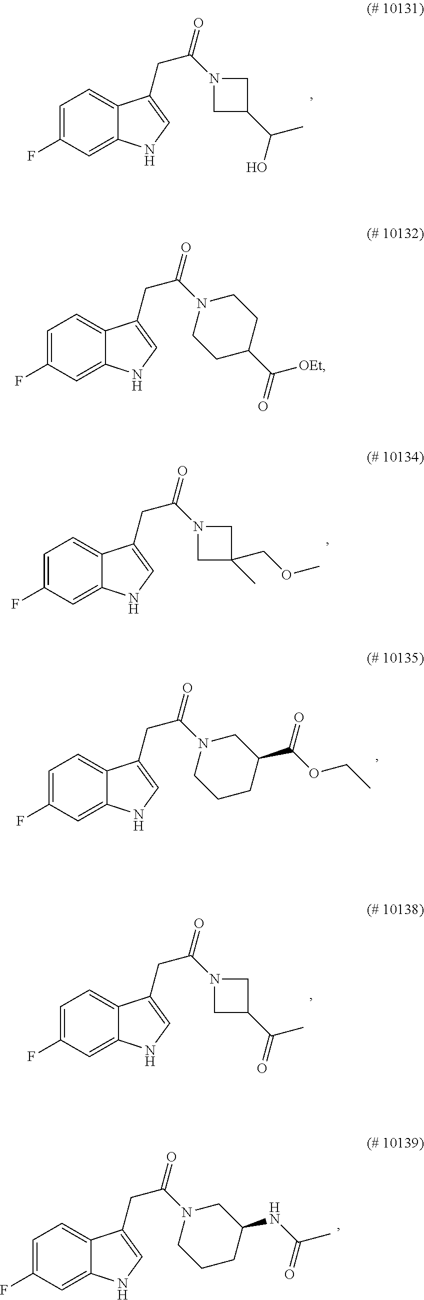 [SMILES: CC(O)C1CN(C(=O)Cc2c[nH]c3cc(F)ccc23)C1 ; CCOC(=O)C1CCN(C(=O)Cc2c[nH]c3cc(F)ccc23)CC1 ; COCC1(C)CN(C(=O)Cc2c[nH]c3cc(F)ccc23)C1 ; CCOC(=O)[C@H]1CCCN(C(=O)Cc2c[nH]c3cc(F)ccc23)C1 ; CC(=O)C1CN(C(=O)Cc2c[nH]c3cc(F)ccc23)C1 ; CC(=O)N[C@H]1CCCN(C(=O)Cc2c[nH]c3cc(F)ccc23)C1] 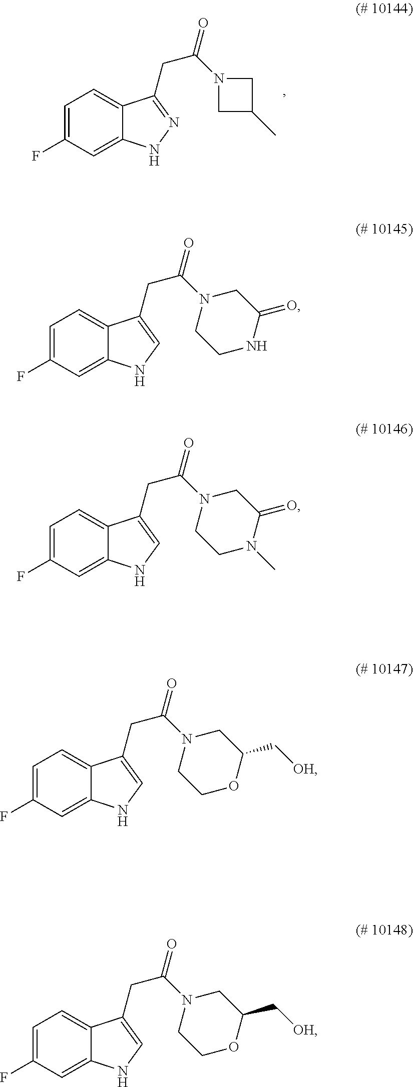 [SMILES: CC1CN(C(=O)Cc2n[nH]c3cc(F)ccc23)C1 ; O=C1CN(C(=O)Cc2c[nH]c3cc(F)ccc23)CCN1 ; CN1CCN(C(=O)Cc2c[nH]c3cc(F)ccc23)CC1=O ; O=C(Cc1c[nH]c2cc(F)ccc12)N1CCO[C@@H](CO)C1 ; O=C(Cc1c[nH]c2cc(F)ccc12)N1CCO[C@H](CO)C1] 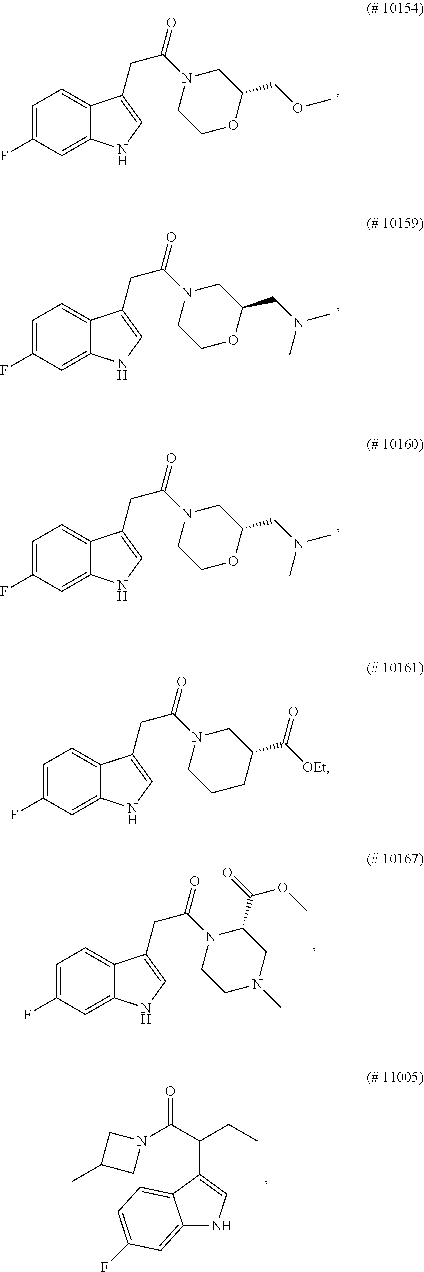 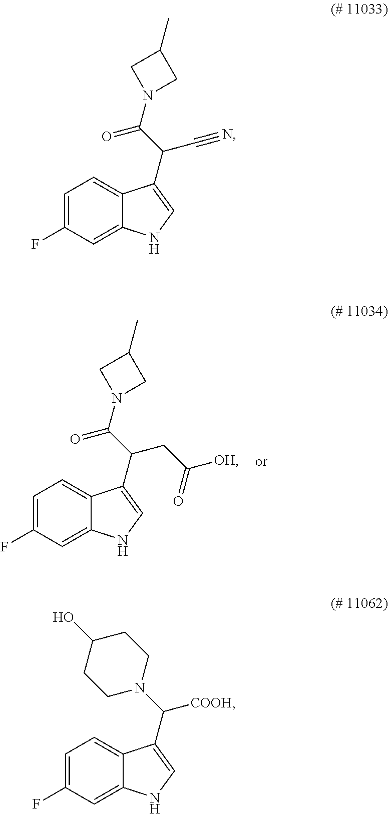 [SMILES: CC1CN(C(=O)C(C#N)c2c[nH]c3cc(F)ccc23)C1 ; CC1CN(C(=O)C(CC(=O)O)c2c[nH]c3cc(F)ccc23)C1 ; O=C(O)C(c1c[nH]c2cc(F)ccc12)N1CCC(O)CC1]
or a pharmaceutically acceptable salt, ester, solvate, isomer, or tautomer thereof.

72. The compound of claim 1 being compound #10040, 10053, 10066, 10076, 10086, 10159, and 11005. Most preferred compound include Compound #10066 or 11005.

73. The compound of any one of claims 1 to 12, wherein D)
R¹⁰ represents H, a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR¹⁵, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵, or
alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each independently unsubstituted or substituted with one or more R¹⁶,
the chain in L is at most 3 atoms in length, contains at least one —C(R¹²)₂— group, and ends with a —N(R¹¹)— group,
the R¹¹ of the —N(R¹¹)— group that ends the chain in L and one R¹² of said at least one —C(R¹²)₂— group together with the one or more atoms to which they are attached and any atom(s) between said one or more atoms form heterocycloalkyl, heterocycloalkanonyl, heterocycloalkenonyl, heterocycloalkynonyl, heterocycloalkenynonyl, or heteroaryl, each of which being independently unsubstituted or substituted with:
a halogen atom, —CN, hydroxyl, —N(R¹⁵)₂, —N(R¹⁵)—C(═O)—R¹⁵, —C(═O)—N(R¹⁵)₂, —C(═O)—R¹⁵, —C(═O)—OR¹⁵, —OR¹⁵, —O—C(═O)—R¹⁵, —SO₂—R¹⁵, —SO—R¹⁵, —N(R¹⁵)₂—SO₂—R¹⁵, or —N(R¹⁵)₂—SO—R¹⁵,
alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which being independently unsubstituted or substituted with one or more R¹⁶,
with the proviso that when said one R¹¹ and said one R¹² form pyrrolidinyl, the pyrrolidinyl is substituted,
with the proviso that when the heterocycloalkyl is piperidinyl, R¹⁰ is other than 2,4-difluorophenylcarbonyl substituent,
any other R¹¹ independently represent H or
alkyl, alkenyl, alkynyl, alkenynyl, each of which independently unsubstituted or substituted with one or more R³¹, and
any other R¹² independently represent H, —C≡N, or —N(R¹⁵)₂,
alkyl, alkenyl, alkynyl, or alkenynyl, alkylamino, alkenylamino, alkynylamino, alkenynylamino, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, heterocycloalkanonyl, heterocycloalkenonyl, heterocycloalkynonyl, heterocycloalkenynonyl, aryl, or heteroaryl, each of which independently unsubstituted or substituted with one or more R³¹,
with the proviso that -L-R¹⁰ is not or

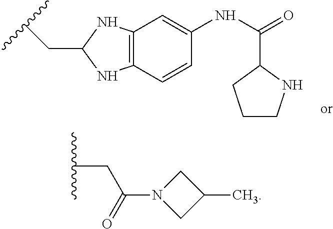 [SMILES: *CC1Nc2ccc(NC(=O)C3CCCN3)cc2N1 ; *CC(=O)N1CC(C)C1]

74. The compound of claim 73, wherein L represents:
—(C(R¹²)₂-)ₙ-C(═O)—N(R¹¹)—, wherein n is 1 or 2, or
—(C(R¹²)₂-)ₙ-N(R¹¹)—, wherein n is 2 or 3,
wherein the R¹¹ and one of the R¹² of these groups together with the two atoms to which they are attached and any atom between said two atoms form the heteroaryl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl, each of which being independently unsubstituted or substituted as described in claim 1.

75. The compound of claim 73, wherein L represents:
—C*(R¹²)₂—C(═O)—N(R¹¹)—,
—C*(R¹²)₂—C(R¹²)₂—C(R¹²)₂—N(R¹¹)—, or
—C*(R¹²)₂—C(R¹²)₂—N(R¹¹)—,
wherein one of the R¹¹ and one of the R¹² (preferably one of the R¹² attached to the carbon atom indicated with a *) together with the two atoms to which they are attached and any atom between said two atoms form the heteroaryl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl, each of which being independently unsubstituted or substituted as described in claim 1.

76. The compound of any one of claims 72 to 75, wherein the R¹² that form the heteroaryl, heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl is attached to the carbon atom in L closest to G, J, X or Q, i.e. the carbon atoms indicated by a star.

77. The compound of any one of claims 72 to 76, wherein the R¹¹ and the R¹² form a heterocycloalkyl, heterocycloalkenyl, heterocycloalkynyl, heterocycloalkenynyl, heterocycloalkanonyl, heterocycloalkenonyl, heterocycloalkynonyl, or heterocycloalkenynonyl,
preferably a heterocycloalkyl or a heterocycloalkanonyl,
more preferably pyrrolidinyl (preferably pyrrolidin-3-yl), succinimidyl, (preferably succinimid-3-yl), or piperidinyl (preferably piperidin-4-yl).

78. The compound of any one of claims 72 to 77, wherein the R¹¹ and the R¹² form pyrrolidinyl (preferably pyrrolidin-3-yl).

79. The compound of any one of claims 72 to 77, wherein the R¹¹ and the R¹² form succinimidyl (preferably succinimid-3-yl).

80. The compound of any one of claims 72 to 77, wherein the R¹¹ and the R¹² form piperidinyl (preferably piperidin-4-yl).

81. The compound of any one of claims 72 to 80, wherein any other R¹¹ represents H.

82. The compound of any one of claims 72 to 81, wherein any other R¹² represents H.

83. The compound of any one of claims 72 to 82, wherein R¹⁰ represents H or —C(═O)—R¹⁵, preferably —C(═O)—R¹⁵, more preferably R¹⁰ represents H or —C(═O)—R²⁰, most preferably —C(═O)—R²⁰.

84. The compound of any one of claims 72 to 83, wherein, R¹⁵ (or R²⁰ as the case may be) represents one of the following groups:
alkyl, alkenyl, alkynyl, alkenynyl, cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, aryl, or heteroaryl,
preferably cycloalkyl, cycloalkenyl, cycloalkynyl, cycloalkenynyl, cycloalkanonyl, cycloalkenonyl, cycloalkynonyl, cycloalkenynonyl, heterocycloalkyl, heterocycloalkenyl, heterocycloalkynyl, heterocycloalkenynyl, aryl, or heteroaryl, each of which preferably being 5- or 6-membered,
more preferably cycloalkyl, cycloalkenyl, cycloalkynyl, cycloalkenynyl, heterocycloalkyl, heterocycloalkenyl, heterocycloalkynyl, heterocycloalkenynyl, or aryl, each of which preferably being 5- or 6-membered, yet more preferably cycloalkyl, heterocycloalkyl, or aryl, each of which preferably being 5- or 6-membered, each of which independently unsubstituted or substituted with one or more R¹⁶. Preferred such cycloalkyls include cyclohexyl. Preferred such heterocycloalkyls include piperidinyl (preferably piperidin-4-yl or piperidin-3-yl) and pyrrolidinyl (preferably pyrrolidin-3-yl). Preferred such aryls include phenyl.

85. The compound of any one of claims 72 to 84, wherein the group in R¹⁵ is unsubstituted.

86. The compound of any one of claims 72 to 85, wherein the group in R¹⁵ is heterocycloalkyl, heterocycloalkenyl, heterocycloalkynyl, heterocycloalkenynyl, or heteroaryl, R¹⁶ is preferably substituting a heteroatom of these groups (preferably N).

87. The compound of any one of claims 72 to 86, wherein R¹⁶ is:
a halogen atom, alkylcarbonyl, alkenylcarbonyl, alkynylcarbonyl, alkenynylcarbonyl, alkoxycarbonyl, alkylcarbamoyl, alkenylcarbamoyl, alkynylcarbamoyl, alkenynylcarbamoyl, or hydroxyl, preferably a halogen atom, alkylcarbonyl, alkoxycarbonyl, alkylcarbamoyl, or hydroxyl, more preferably a halogen atom, alkylcarbonyl, alkylcarbamoyl, or hydroxyl.

88. The compound of any one of claims 72 to 87, wherein when R¹⁶ is substituting a nitrogen atom, R¹⁶ is alkylcarbonyl or alkoxycarbonyl, preferably alkylcarbonyl.

89. The compound of claim 1 being:

90. 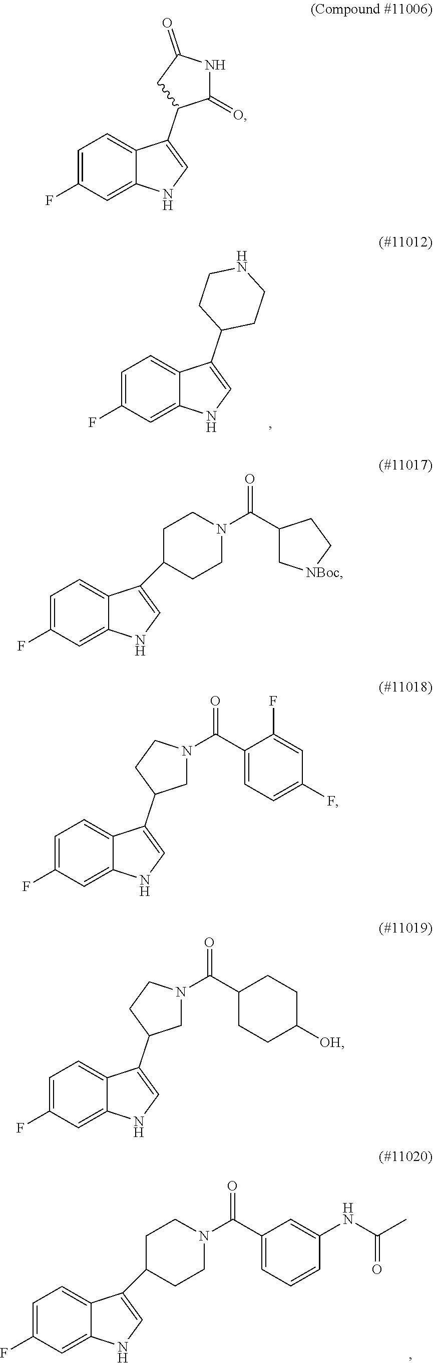 [SMILES: O=C1CC(c2c[nH]c3cc(F)ccc23)C(=O)N1 ; Fc1ccc2c(C3CCNCC3)c[nH]c2c1 ; CC(C)(C)OC(=O)N1CCC(C(=O)N2CCC(c3c[nH]c4cc(F)ccc34)CC2)C1 ; O=C(c1ccc(F)cc1F)N1CCC(c2c[nH]c3cc(F)ccc23)C1 ; O=C(C1CCC(O)CC1)N1CCC(c2c[nH]c3cc(F)ccc23)C1 ; CC(=O)Nc1cccc(C(=O)N2CCC(c3c[nH]c4cc(F)ccc34)CC2)c1] 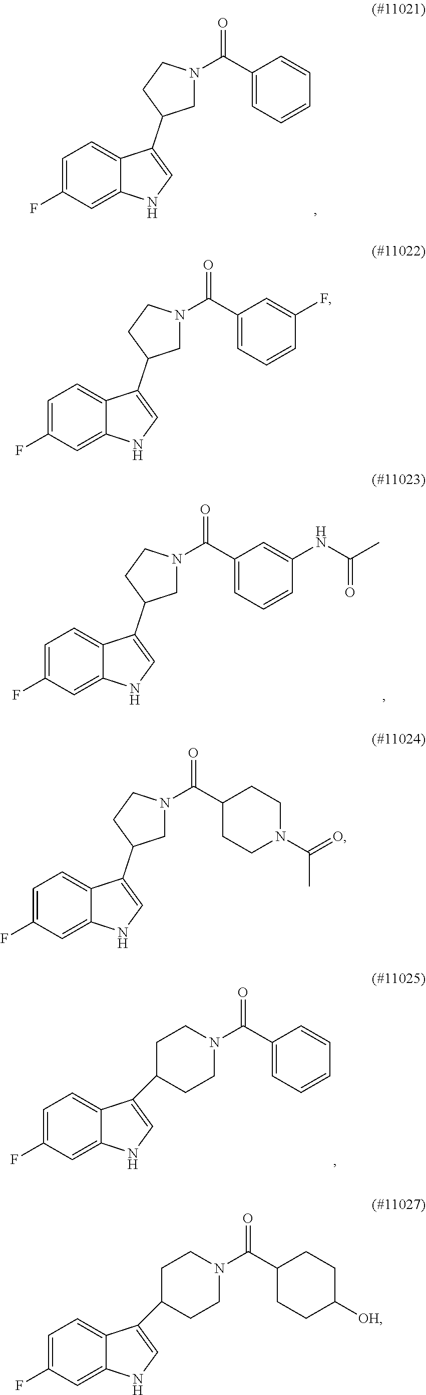 [SMILES: O=C(c1ccccc1)N1CCC(c2c[nH]c3cc(F)ccc23)C1 ; O=C(c1cccc(F)c1)N1CCC(c2c[nH]c3cc(F)ccc23)C1 ; CC(=O)Nc1cccc(C(=O)N2CCC(c3c[nH]c4cc(F)ccc34)C2)c1 ; CC(=O)N1CCC(C(=O)N2CCC(c3c[nH]c4cc(F)ccc34)C2)CC1 ; O=C(c1ccccc1)N1CCC(c2c[nH]c3cc(F)ccc23)CC1 ; O=C(C1CCC(O)CC1)N1CCC(c2c[nH]c3cc(F)ccc23)CC1] 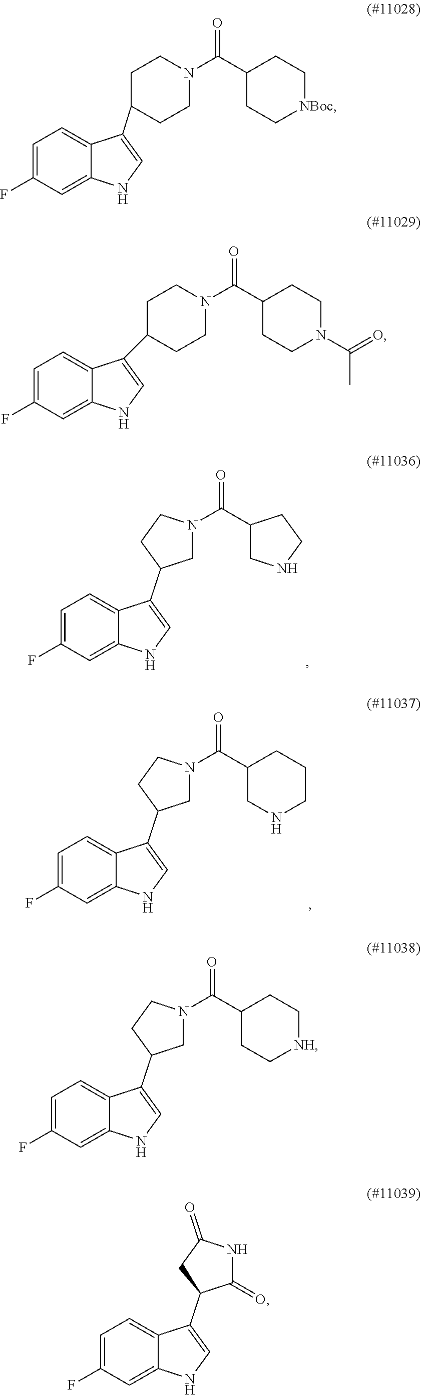 [SMILES: CC(C)(C)OC(=O)N1CCC(C(=O)N2CCC(c3c[nH]c4cc(F)ccc34)CC2)CC1 ; CC(=O)N1CCC(C(=O)N2CCC(c3c[nH]c4cc(F)ccc34)CC2)CC1 ; O=C(C1CCNC1)N1CCC(c2c[nH]c3cc(F)ccc23)C1 ; O=C(C1CCCNC1)N1CCC(c2c[nH]c3cc(F)ccc23)C1 ; O=C(C1CCNCC1)N1CCC(c2c[nH]c3cc(F)ccc23)C1 ; O=C1C[C@H](c2c[nH]c3cc(F)ccc23)C(=O)N1] 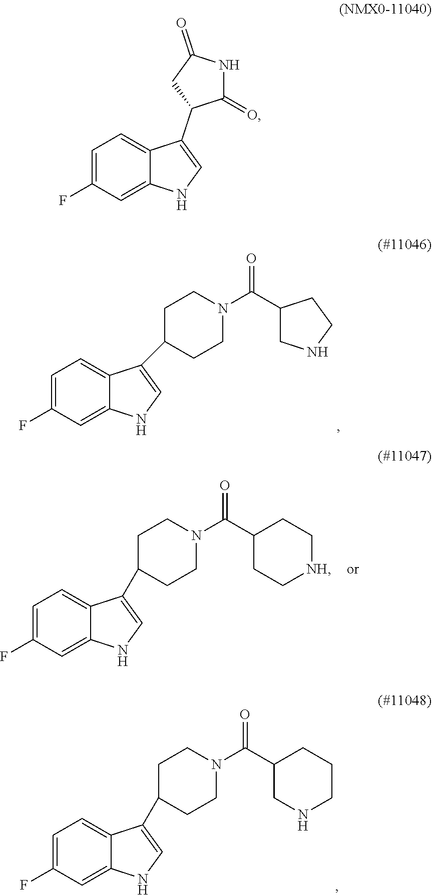 [SMILES: O=C1C[C@@H](c2c[nH]c3cc(F)ccc23)C(=O)N1 ; O=C(C1CCNC1)N1CCC(c2c[nH]c3cc(F)ccc23)CC1 ; O=C(C1CCNCC1)N1CCC(c2c[nH]c3cc(F)ccc23)CC1 ; O=C(C1CCCNC1)N1CCC(c2c[nH]c3cc(F)ccc23)CC1]
or a pharmaceutically acceptable salt, ester, solvate, isomer, or tautomer thereof.

91. The compound of claim 1 being compound #11006.

92. The compound of any one of claims 1 to 91 being of formula (II):

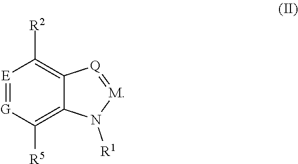

93. The compound of claim 92, wherein:
E represents —CH═,
G represents —C(R⁴)═, preferably wherein R⁴ represents a halogen atom, more preferably F,
M represents —N═ or —C(R⁷)═,
Q represents —C(R⁸)═, preferably —C(L-R¹⁰)═, wherein L and R¹⁰ are as defined in any one of claims 1 to 91,
R¹ represents H,
R² represents H, and/or
R⁴ represents H, and
with the proviso that the compound of formula (II) comprises exactly one -L-R¹⁰ group,
or a pharmaceutically acceptable salt, ester, solvate, isomer, or tautomer thereof.

94. The compound of claim 92, wherein M represents —C(R⁷)═, preferably —CH═.

95. The compound of claim 92, wherein, M represents —N═.

96. The compound of claim 92 being of formula (III): 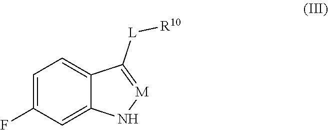
or a pharmaceutically acceptable salt, ester, solvate, isomer, or tautomer thereof.

97. The compound of claim 96, wherein M represents —C(R⁷)═, preferably —CH═.

98. The compound of claim 96, wherein M represents —N═.

99. The compound of claim 96 being of formula (IV): 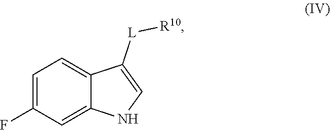
or a pharmaceutically acceptable salt, ester, solvate, isomer, or tautomer thereof.

100. The compound of claim 1 being:

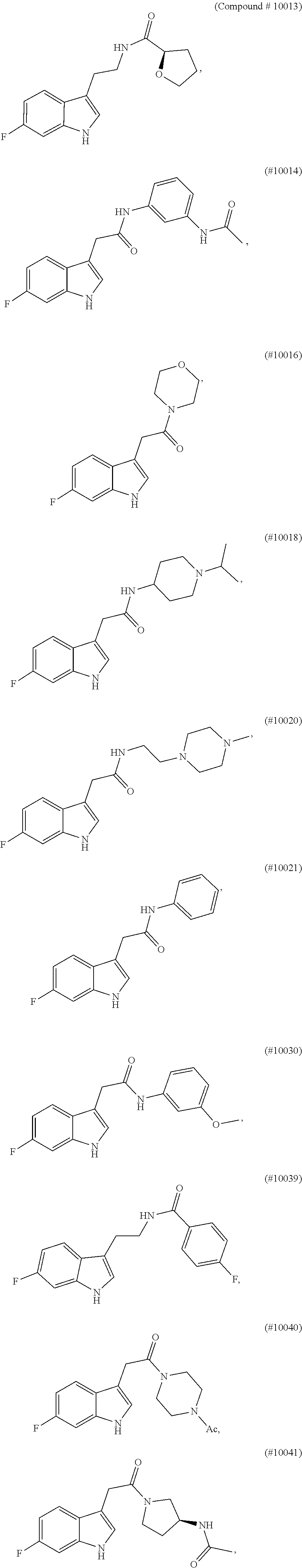

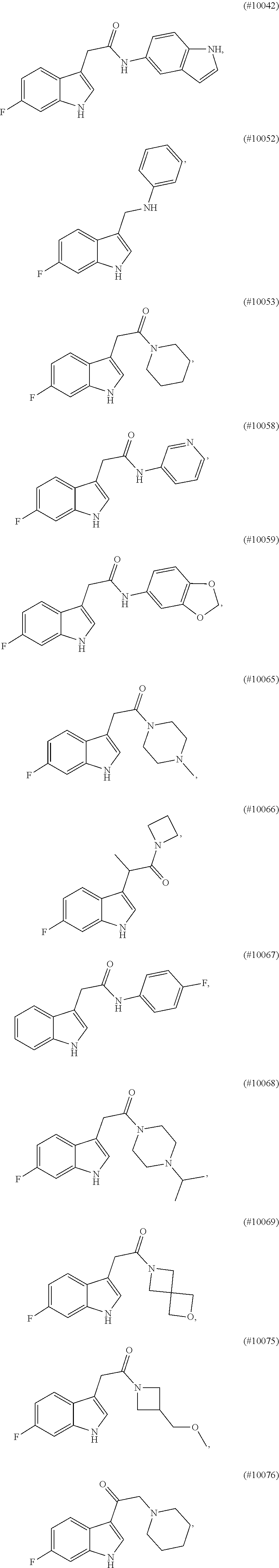

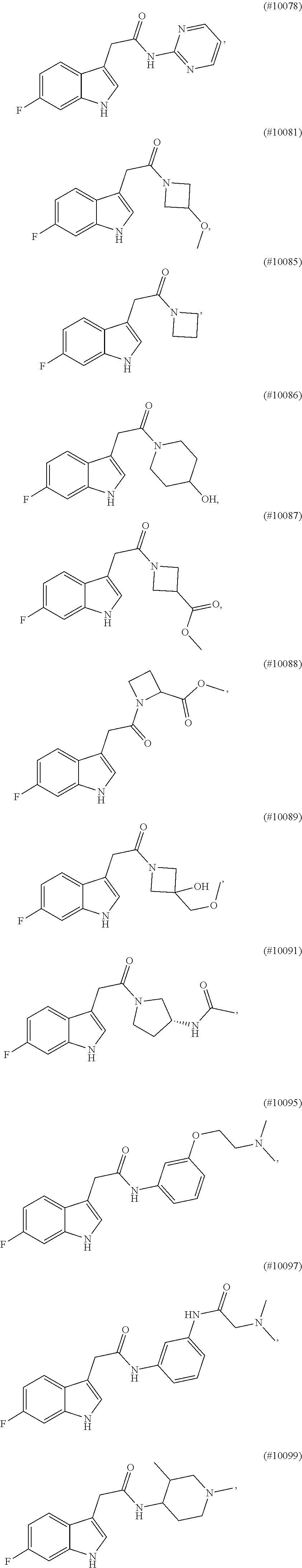

101.

102. 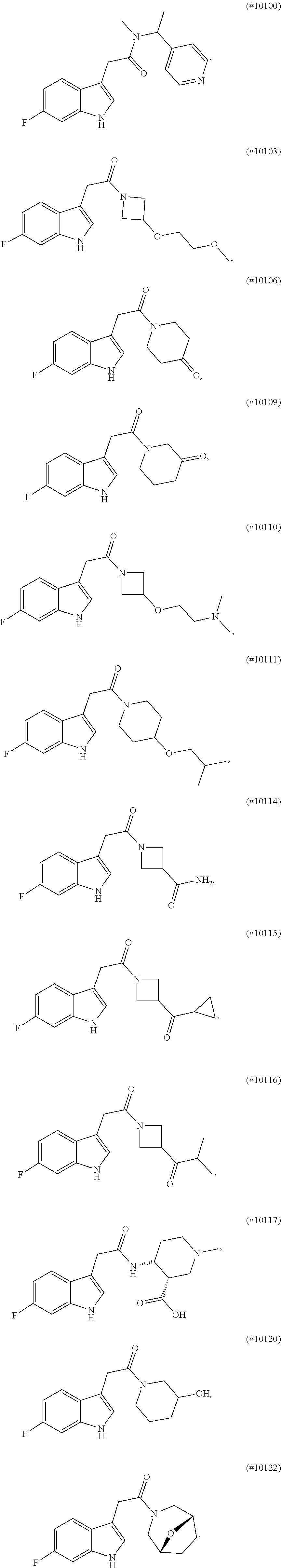 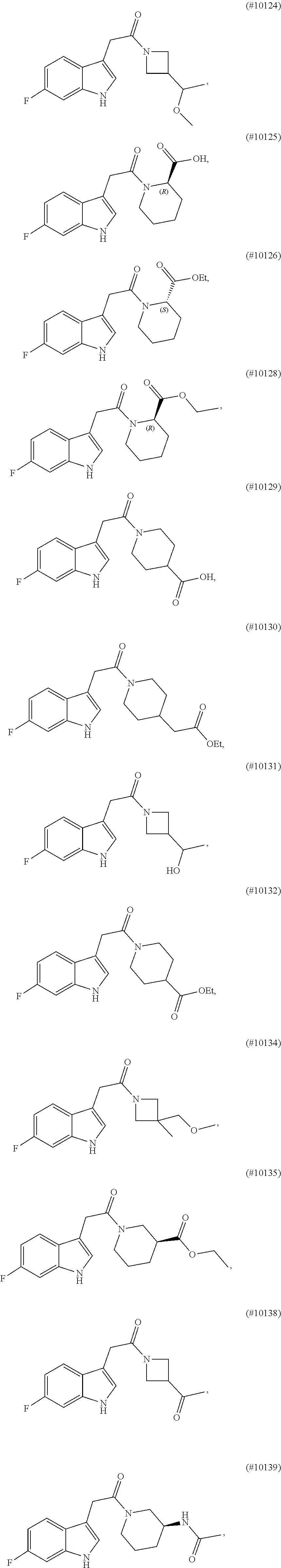 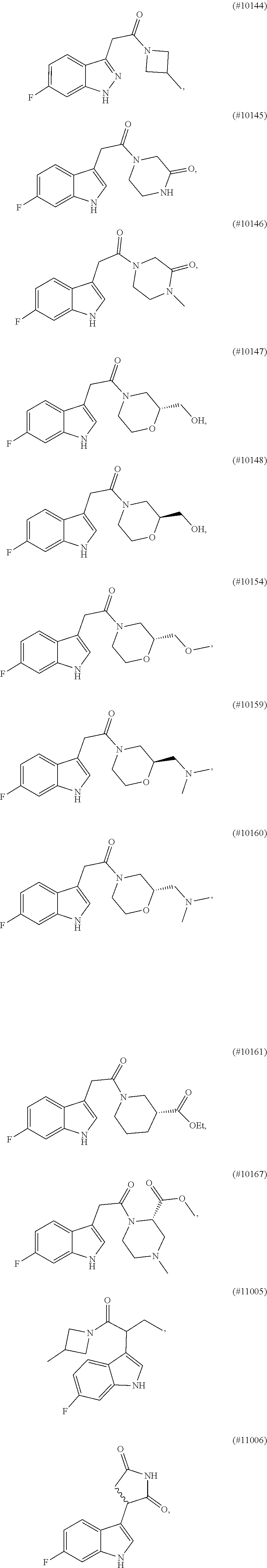 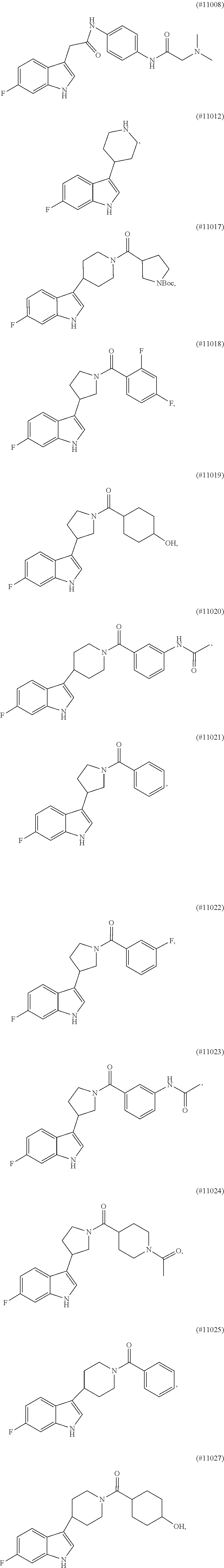 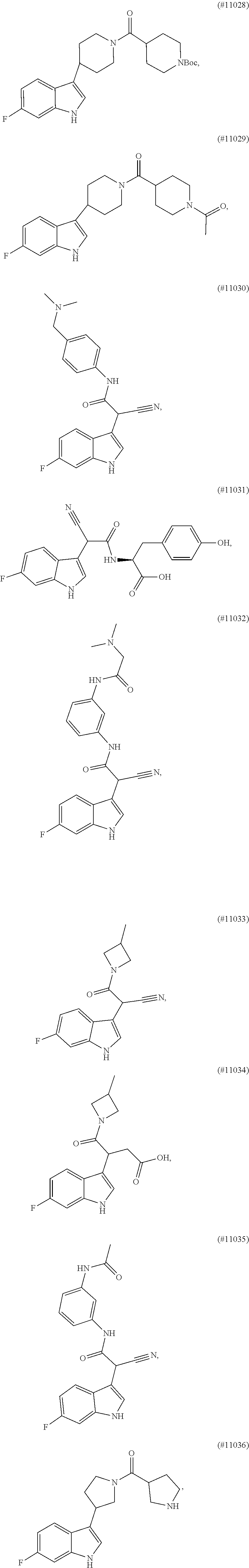 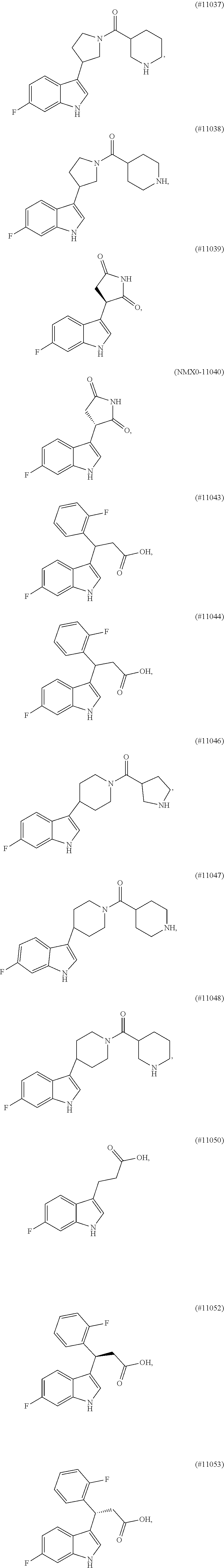 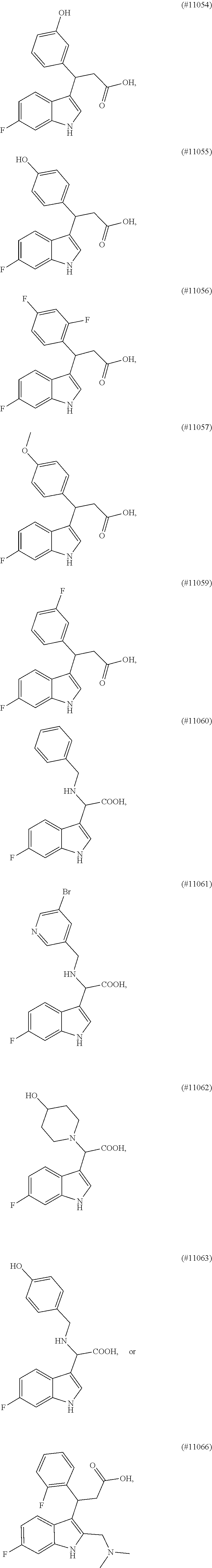
or a pharmaceutically acceptable salt, ester, solvate, isomer, or tautomer thereof.

103. The compound of claim 1 being Compound #10014, 10018, 10035, 10040, 10053, 10066, 10076, 10086, 10095, 10097, 10159, 11005, 11006, 11008, 11043, 11052 (enantiomer of 11043), 11053 (enantiomer of 11043), 11054, 11055, 11066, or 11073, or a pharmaceutically acceptable salt, ester, solvate, isomer, or tautomer thereof.

104. The compound of claim 1 being Compound #10018, 10040, 10086, 10095, 10097, 11005, 11006, 11008, 11032, 11053, 11066, or 11073, or a pharmaceutically acceptable salt, ester, solvate, isomer, or tautomer thereof.

105. The compound of claim 1 being Compound #10095, 10097, 11005, 11006, 11008, or 11053, or a pharmaceutically acceptable ester, solvate, isomer, or tautomer thereof.

106. The compound of claim 1 being Compound #11095, 11097, or 11032, or a pharmaceutically acceptable ester, solvate, isomer, or tautomer thereof.

107. The compound of claim 1 being Compound #10018, 10040, 10086, or 11066, or a pharmaceutically acceptable ester, solvate, isomer, or tautomer thereof.

108. A method for inhibiting RAS (wild type or mutant), for example pan-RAS, HRAS, NRAS, and/or KRAS, preferably inhibiting HRAS, and more preferably selectively inhibiting HRAS (or in alternative embodiments selectively inhibiting NRAS, or in yet other alternative embodiments selectively inhibiting KRAS) in a subject in need thereof comprising administering to the subject an effective amount of the compound of any one of claims 1 to 107.

109. Use of the compound of any one of claims 1 to 107 for inhibiting RAS (wild type or mutant), for example pan-RAS, HRAS, NRAS, and/or KRAS, preferably inhibiting HRAS, and more preferably selectively inhibiting HRAS (or in alternative embodiments selectively inhibiting NRAS, or in yet other alternative embodiments selectively inhibiting KRAS) in a subject, or for the manufacture of a medicament for inhibiting HRAS in a subject.

110. A compound of any one of claims 1 to 107 for use in inhibiting RAS (wild type or mutant), for example pan-RAS, HRAS, NRAS, and/or KRAS, preferably inhibiting HRAS, and more preferably selectively inhibiting HRAS (or in alternative embodiments selectively inhibiting NRAS, or in yet other alternative embodiments selectively inhibiting KRAS) in a subject.

111. The method, use and compound of any one of claims 108 to 110, being for inhibiting a HRAS mutant, such as HRAS mutated at residue 12.

112. The method, use and compound of claim 111, being for inhibiting HRAS G12V mutant.

113. The method, use and compound of any one of claims 108 to 112, being for inhibiting NRAS mutant, such as NRAS mutated at residue 61.

114. The method, use and compound of claim 113, being for inhibiting NRAS Q61R mutant.

115. The method, use and compound of any one of claims 108 to 114, being for inhibiting KRAS mutant, such as KRAS mutated at residue 12.

116. The method, use and compound of claim 115, being for inhibiting KRAS G12C or G12D mutant.

117. The method, use and compound of any one of claims 108 to 116, being for inhibiting a PAN-RAS mutant, such as PAN-RAS mutated at residue 12.

118. The method, use and compound of claim 117, being for inhibiting PAN-RAS G12C or G12D mutant, preferably PAN-RAS G12C mutant.

119. The method, use and compound of any one of claims 108 to 110, being for selectively inhibiting HRAS, including wild type HRAS or mutant HRAS.

120. The method, use and compound of claim 119, being for selectively inhibiting HRAS mutated at position 12.

121. The method, use and compound of claim 120, being for selectively inhibiting HRAS G12V mutant.

122. The method, use and compound of any one of claims 108 to 110, being for selectively inhibiting NRAS, including wild type HRAS or mutant NRAS.

123. The method, use and compound of claim 122, being for selectively inhibiting NRAS mutated at position 61.

124. The method, use and compound of claim 123, being for selectively inhibiting NRAS Q61R mutant.

125. The method, use and compound of any one of claims 108 to 110, being for selectively inhibiting KRAS, including wild type HRAS or mutant.

126. The method, use and compound of claim 125, being for selectively inhibiting KRAS mutated at position 12.

127. The method, use and compound of claim 126, being for selectively inhibiting KRAS G12C or G12D mutant.

128. The method, use and compound of any one of claims 108 to 110, being for selectively inhibiting pan-RAS, including wild type HRAS or mutant wild type pan-RAS or mutant pan-RAS.

129. The method, use and compound of claim 128, being for selectively inhibiting PAN-RAS mutated at position 12.

130. The method, use and compound of claim 129, being for selectively inhibiting PAN-RAS G12C or G12D mutant, preferably PAN-RAS G12C mutant.

131. The method, use and compound of any one of claims 108 to 110, being for the prevention or treatment of a disease or disorder associated with abnormal RAS activity, for example abnormal RAS activity caused by a mutation in RAS, preferably Costello syndrome, epidermal nevus (e.g., epidermal nevus sebaceous), giant congenital melanocytic nevus, Noonan syndrome, Noonan syndrome with multiple lentigines, autoimmune lymphoproliferative syndrome, cardiofaciocutaneous syndrome, neurofibromatosis type 1, capillary malformation-arteriovenous malformation syndrome, Legius syndrome or a cancer with a mutated RAS.

132. The method, use and compound of any one of claims 108 to 110, being the prevention or treatment of a disease or disorder associated with abnormal HRAS activity, for example abnormal HRAS activity caused by a mutation in HRAS, preferably a mutation at position 12, more preferably Costello syndrome, epidermal nevus (e.g., epidermal nevus sebaceous) or a cancer with a mutated HRAS.

133. The method, use and compound of any one of claims 108 to 110, being for the prevention or treatment of a disease or disorder associated with abnormal NRAS activity, for example abnormal NRAS activity caused by a mutation in NRAS, preferably a mutation at position 61, preferably giant congenital melanocytic nevus, Noonan syndrome, autoimmune lymphoproliferative syndrome, epidermal nevus or a cancer with a mutated NRAS.

134. The method, use and compound of any one of claims 108 to 110, being for the prevention or treatment of a disease or disorder associated with abnormal KRAS activity, for example abnormal KRAS activity caused by a mutation in KRAS, preferably a mutation at position 12, more preferably cardiofaciocutaneous syndrome, Noonan syndrome, autoimmune lymphoproliferative syndrome, Epidermal nevus, or a cancer with a mutated KRAS.

135. The method, use and compound of any one of claims 108 to 110, being for the prevention or treatment of a disease or disorder associated with abnormal PAN-RAS activity, for example abnormal PAN-RAS activity caused by a mutation in PAN-RAS, preferably a mutation at position 12, more preferably the disease or disorder as defined in any one of claims 131 to 134.

136. A method for treating cancer in a subject in need thereof comprising administering to the subject an effective amount of the compound of any one of claims 1 to 107.

137. Use of the compound of any one of claims 1 to 107 for treating cancer in a subject, or

138. Use of the compound of any one of claims 1 to 107 for the manufacture of a medicament for treating cancer in a subject.

139. The compound of any one of claims 1 to 107 for use in treating cancer in a subject.

140. The method, use and compound of any one of claims 136 to 139 being for treating a cancer with a mutated HRAS, preferably bladder, breast, colon, colorectal, cutaneous, embryonal rhabdomyosarcoma, endometrial, glioblastoma, head and neck, leukemia, lung, melanoma (including cutaneous melanoma), oral cavity, ovarian, prostate, renal, salivary duct, skin, or thyroid cancer, more preferably head and neck cancer (preferably head and neck squamous cell carcinoma), thyroid cancer, epithelial-myoepithelial carcinoma, kidney cancer or bladder cancer.

141. The method, use and compound of any one of claims 136 to 139 being for treating a cancer with a mutated NRAS, preferably a melanoma (including individuals without giant congenital melanocytic nevus), lung cancer, cholangiocarcinoma, or a hematopoietic malignancy such core binding factor acute myeloid leukemia and cytogenetically normal acute myeloid leukemia, more preferably a melanoma (including individuals without giant congenital melanocytic nevus), lung cancer or a hematopoietic malignancy.

142. The method, use and compound of any one of claims 136 to 139 being for treating a cancer with a mutated KRAS, preferably pancreatic cancer, cholangiocarcinoma, Core binding factor acute myeloid leukemia, colorectal cancer, or lung cancer (including non-small cell lung cancer), and more preferably pancreatic cancer, colorectal cancer, or a lung cancer.

143. The method, use and compound of any one of claims 136 to 139 being for treating a cancer with a mutated PAN-RAS, preferably a cancer as defined in any one of claims 140 to 142.

144. A composition comprising the compound of any one of claims 1 to 107 and a pharmaceutically acceptable carrier or excipient.
